(19) United States
(12) Patent Application Publication
(10) Pub. No.: US 2015/0352108 A1
(43) Pub. Date: Dec. 10, 2015

(54) AMINO-PYRIMIDINE COMPOUNDS AS INHIBITORS OF TBK1 AND/OR IKK EPSILON

(71) Applicant: Alzheimer's Institute of America, Kansas City, KS (US)

(72) Inventors: Ryan C. Holcomb, Salt Lake City, UT (US); Kazuyuki Suzuki, Fukushima (JP); Robert J. Halter, Salt Lake City, UT (US); Paul R. Sebahar, Sandy, UT (US); Donald A. McLeod, Sandy, UT (US); Mark D. Shenderovich, Salt Lake City, UT (US); Kraig M. Yager, Murray, UT (US); Matthew Gregory Bursavich, Needham, MA (US); Ashantai J. Yungai, Salt Lake City, UT (US); Burt Richards, Midway, UT (US); Paul L. Bartel, Salt Lake City, UT (US); Daniel A. Wettstein, Salt Lake City, UT (US)

(73) Assignee: Alzheimer's Institute of America, Kansas City, KS (US)

(21) Appl. No.: 14/581,065

(22) Filed: Dec. 23, 2014

Related U.S. Application Data
(63) Continuation of application No. 14/273,387, filed on May 8, 2014, now abandoned.
Application No. 14/273,387 is a continuation of application No. 14/027,041, filed on Sep. 13, 2013, now abandoned.
Application No. 14/027,041 is a continuation of application No. 13/445,627, filed on Apr. 12, 2012, now abandoned.
Application No. 13/445,627 is a continuation of application No. PCT/US2010/052385, filed on Oct. 12, 2010.
(60) Provisional application No. 61/250,842, filed on Oct. 12, 2009.
(60) Provisional application No. 61/325,245, filed on Apr. 16, 2010.

(51) Int. Cl. A61K 31/505; A61K 31/506; A61K 31/52; A61K 31/551; A61K 31/541; A61K 31/5377; A61K 31/635

(52) U.S. Cl. CPC A61K 31/505; A61K 31/5377; A61K 31/506; A61K 31/635; A61K 31/551; A61K 31/541; A61K 31/52

(57) ABSTRACT

The invention relates to certain amino-pyrimidine compounds which inhibit TBK1 and/or IKK epsilon and which may therefore find application in treating inflammation, cancer, septic shock and/or Primary open Angle Glaucoma (POAG).

### RELATED APPLICATIONS

This application is a continuation of U.S. patent application Ser. No. 14/273,387, filed May 8, 2014; which is a continuation of U.S. patent application Ser. No. 14/027,041, filed Sep. 13, 2013; which is a continuation of U.S. patent application Ser. No. 13/445,627, filed Apr. 12, 2012, and published as U.S. Publication No. 2012-0238540; which is a continuation of International Patent Application No. PCT/US2010/052385, filed Oct. 12, 2010, and published as WO 2011/046970; which claims the benefit of U.S. Provisional Application Ser. No. 61/250,842, filed Oct. 12, 2009 and U.S. Provisional Application Ser. No. 61/325,245, filed Apr. 16, 2010; the contents of all of which are incorporated by reference herein in their entirety.

### FIELD OF THE INVENTION

The present invention relates generally to the field of medicinal chemistry. Specifically, the present invention provides compounds that inhibit IKK-related kinase epsilon (IKKε), TANK-binding kinase 1 (TBK1), or both IKKε and TBK1. The invention also provides methods for making these compounds, pharmaceutical compositions comprising these compounds, and methods for treating diseases with these compounds and compositions.

### BACKGROUND OF THE INVENTION

The protein “I-kappa-B kinase epsilon” or “IKKε” (also known as “inducible IkappaB kinase” or “IKK-i”) is a member of the IκB family of kinases, and contains a kinase domain in its N-terminus, which shares substantial identity to that of I-kappa-B kinase alpha (IKKα) or I-kappa-B kinase beta (IKKβ), and even greater identity with the kinase domain of TANK-binding kinase 1 (TBK1). IKKε was first identified as a protein whose encoding messenger RNA is substantially induced by lipopolysaccharide (LPS). (Shimada, et al.; IKK-i, a novel lipopolysaccharide-inducible kinase that is related to IκB kinases; Int. Immunol., 11:1357-1362, 1999.) Subsequent studies revealed that the expression of IKKε is induced by activation of the inflammatory NF-κB signaling pathway. (Matsuda, et al.; Large-scale identification and characterization of human genes that activate NF-kappaB and MAPK signaling pathways; Oncogene, 22:3307-3318, 2003.) IKKε is expressed mainly in immune cells, and is induced in response to pro-inflammatory cytokines such as tumor necrosis factor-alpha, IL-1 and IL-6, in addition to lipopolysaccharide (LPS). Overexpression of wild-type IKKε results in the phosphorylation of IκB alpha, and stimulation of NF-kappaB activation. (Shimada, et al.; Int. Immunol., 11:1357-1362, 1999.)

While all of its functions are not completely understood, IKKε has been found to play many important roles in human cells. For example, it has been known for some time that IKKε plays a key role in integrating signals induced by pro-inflammatory stimuli. (Kravchenko et al., IKKi/IKKepsilon plays a key role in integrating signals induced by pro-inflammatory stimuli; J. Biol. Chem., 278:26612-26619, 2003.) Further, it is known that IKKε is involved in the antiviral interferon (IFN) response, and that, along with TBK1, IKKε forms a virus-activated kinase complex that phosphorylates interferon regulatory factors 3 and 7 (IRF3 & IRF7). (Sharma et al.; Triggering the interferon antiviral response through an IKK-related pathway; Science, 300:1148-1151, 2003.) Additionally, IKKε, along with TBK1, has been shown to play a role in maintaining macrophages in an activated, inflammatory state, following activation of the interferon response. (Solis, et al.; Involvement of TBK1 and IKKepsilon in lipopolysaccharide-induced activation of the interferon response in primary human macrophages; Eur. J. Immunol., 37:529-539, 2007.)

TBK1 is highly related to IKKε and is constitutively expressed in most cell types (Clement et al., The IKK-related kinases: from innate immunity to oncogenesis; Cell Res., 18:889-899, 2008). Similar to IKKε, TBK1 is responsible for phosphorylation of IRF3 & IRF7 and NF-kB transcription factors after activation of innate immune receptors leading to transcription of several proinflammatory proteins (Chau et al., Are the IKKs and IKK-related kinases TBK1 and IKK-epsilon similarly activated?; Trends Biochem Sci., 33:171-180, 2008). TBK1 and IKKε protein share redundant and possibly overlapping roles in innate immune signaling and possibly autoimmune diseases, therefore inhibition of both kinases may prove advantageous.

In view of the roles identified for IKKε in the interferon antiviral response, and in the maintenance of macrophages in an activated, inflammatory state, it is perhaps not surprising that IKKε, as part of the kinase complex, has also been found to play a role in the synovial inflammation, extracellular matrix destruction and activation of the viral program and innate immune response in rheumatoid arthritis (RA). (Sweeney et al., Regulation of c-Jun phosphorylation by the IκB kinase-ε complex in fibroblast-like synoviocytes; J. Immunol., 174:6424-6430, 2005.) Indeed, further studies of the role of IKKε and its downstream phosphorylation target IRF3 in RA, have demonstrated that IKKε and IRF3 protein levels are significantly elevated in RA synovium compared to osteoarthritic synovium, and that an IKKε-dependent mechanism results in the increased production of interferon beta, and RANTES in cultured synoviocytes. IKKε null mice demonstrated reduced inflammation and erosion as well as a decrease in clinical arthritis in the collagen-induced arthritis model (Corr et al.; Synergistic benefit in inflammatory arthritis by targeting IκB kinase E and interferon β; Ann. Rheum. Dis., 68:257-263, 2009). These results suggest that the IKKε-dependent pathway may be an important therapeutic target in the treatment of RA. (Sweeney et al.; Antiviral gene expression in rheumatoid arthritis; Arthritis Rheum., 56:743-752, 2007).

Systemic lupus erythematosus (SLE) is an autoimmune disease principally affecting women of child-bearing age. The disease is caused by an inappropriate immune response directed against intranuclear, self-antigens. It manifests systemically with involvement of many organs, including the kidneys, joints, skin and nervous system. The underlying inflammatory state predisposes patients to infections and cardiovascular disease, which are the major causes of mortality and morbidity in SLE. The current model for the molecular pathology of SLE is deregulation of T, B, and dendritic cell populations via an undetermined mechanism. This leads to imbalances of several cytokines and chemokines in T and B cell compartments eventually leading to organ damage (Crispin et al.; Pathogenesis of human systemic lupus erythematosus: recent advances; Trends Mol. Med., 16:47-57, 2010). In addition, the inability of dendritic cells to properly integrate signals from apoptotic cell debris or bacterial and viral infections leads to overproduction of the type I interferons (IFNα/β). In approximately half of all SLE patients a characteristic interferon gene signature has been identified (Baechler et al.; Interferon-inducible gene expression signature in peripheral blood cells of patients with severe lupus; Proc. Natl. Acad. Sci. U.S.A., 100:2610-2615, 2003). The expression of many of the interferon-regulated genes coincides with flares or periods of increased disease symptoms in SLE patients. While a single underlying cause has not been described to date, it is clear that adaptive and innate immune responses are compromised which leads to aberrant regulation of the entire immune system in SLE patients. The increase in IFNα/β production in SLE patients is due to activation of toll-like receptors (TLRs) and possibly intracellular nucleic acid receptors (Baccala et al.; TLR-dependent and TLR-independent pathways of type I interferon induction in systemic autoimmunity; Nat. Med., 13:543551, 2007). One of the downstream effects of receptor engagement is activation of the IKKε and TBK1 kinases leading to phosphorylation of transcription factors IRF3 and IRF7. Upon phosphorylation, the IRFs move into the nucleus and mediate upregulation of IFNα/β and associated interferon signature genes, including OAS1, OAS2, MX1, MX2, PKR, ISG54, ISG56, RANTES, CXCL-10, as well as others.

IKKε and TBK1 are involved in autoimmune diseases associated with accumulation of cytosolic nucleic acids. Several autoimmune diseases including; Sjögrens syndrome, Aicardi-Goutieres syndrome, subtypes of SLE, chilblain lupus, retinal vasculopathy and cerebral leukodystrophy (RVCL) appear to be caused by mutations in genes such as TREX1, SAMHD1, and RNASEH2A-C, which encode proteins involved in degrading viral nucleic acids or accumulated endogenous cytosolic nucleic acids (Crow and Rehwinkel; Aicardi-Goutières syndrome and related phenotypes: linking nucleic acid metabolism with autoimmunity; Hum. Mol. Genet., 18; 130-136, 2009; and Kavanagh, et al.; New roles for the major human 3′-5′ exonuclease TREX1 in human disease; Cell Cycle, 7:1718-1725, 2008). Patients carrying mutations that result in reduction or complete loss of protein activity have elevated expression of IFNβ and a set of “interferon signature” genes, and this elevated expression is dependent on IRF3 (Stetson et al.; Trex1 prevents cell-intrinsic initiation of autoimmunity; Cell, 134:587-598, 2008). IRF3 is phosphorylated by IKKε and/or TBK1 in response to signals from nucleic acid receptors, such as RIG-I, MDA5, DAI, IFI16, and others (Unterholzner et al.; IFI16 is an innate immune sensor for intracellular DNA; Nat. Immunol., E-pub Oct. 3, 2010), and phosphorylation of IFR3 leads to type I interferon production.

Systemic sclerosis, Sjögrens syndrome, dermatomyositis, polymyositis (Walsh et al.; Type I Interferon-Inducible Gene Expression in Blood Is Present and Reflects Disease Activity in Dermatomyositis and Polymyositis; Arthritis Rheum., 56:3784-3792, 2007) and plaque psoriasis (Delgado-Vega, et al.; Genetic associations in type I interferon related pathways with autoimmunity; Arthritis Res. Ther., April 14; 12 Suppl 1:S2, 2010) are autoimmune diseases characterized by elevated type I interferons and a characteristic interferon gene signature (Sozzani, et al.; Type I interferons in systemic autoimmunity; Autoimm., 43:196-203, 2010). Signaling pathways involving IKKε and TBK1 increase type I interferon expression following activation of upstream TLR3, TLR4, and cytosolic nucleic acid receptors (Honda et al.; Regulation of the type I IFN induction: a current view; Intern. Immunol, 17:1367-1378, 2005) consistent with a role in systemic sclerosis and myositis. Increased type I IFN signaling and the upregulation of viral dsRNA receptors including; TLR3, RIG1, and MDA5 in psoriatic skin support a role for IKKε and TBK1 in the pathogenesis of psoriasis (Prens et al.; IFN-alpha enhances poly-IC responses in human keratinocytes by inducing expression of cytosolic innate RNA receptors: relevance for psoriasis; J. Invest. Dermatol., 128: 932-938, 2008).

Chronic obstructive pulmonary disease (COPD) is characterized by inflammation of the lungs and narrowing of the airways. Exacerbation of COPD is caused by viral and bacterial infections that can prove fatal. Viral and bacterial pulmonary infections are recognized by toll-like receptors or cytosolic nucleic acid receptors (Takaoka and Taniguchi; Cytosolic DNA recognition for triggering innate immune response; Adv. Drug Delivery Rev., 60:847-857, 2008), which activate IKKε and TBK1 kinases and lead to proinflammatory response. The involvement of IKKε and TBK1 kinases in this response is supported by findings that several IRF3 and IRF7 responsive proinflammatory genes (e.g., IFNβ, IP-10 and IL-8) are induced during rhinovirus-induced COPD (Wang et al.; Role of double-stranded RNA pattern recognition receptors in rhinovirus-induced airway epithelial cell responses; J. Immunol., 183:6989-6997, 2009).

Inflammatory bowel disease (IBD) is an autoimmune-like disease characterized by an abnormal response to bacteria in the gut. TLRs have been implicated in IBD based on single-nucleotide polymorphisms in IBD patients (Cario; Toll-like receptors in inflammatory bowel diseases: a decade later; Inflamm. Bowel Dis., 16:1583-1597, 2010). The TLR4 protein is a bacterial lipopolysaccharide-recognizing receptor that activates the IRF3 pathway through IKKε and TBK1 kinases leading to RANTES and MCP-1 secretion. Elevation of both RANTES and MCP-1 protein levels are associated with IBD (McCormack et al.; Tissue cytokine and chemokine expression in inflammatory bowel disease; Inflamm. Res., 50:491-495, 2001).

It has been shown that a high-fat diet can increase NF-κB activation in mice, which leads to sustained elevation in the level of IKKε in liver, adipocytes, and adipose tissue macrophages. (See Chiang et al.; The protein kinase IKKε regulates energy balance in obese mice; Cell, 138:961-975, 2009) Further, mice in which the gene encoding IKKε was knocked out were found to be protected from high-fat diet-induced obesity, chronic inflammation in liver and fat, hepatic steatosis, and whole-body insulin resistance. These IKKε knockout mice were found to have increased energy expenditure and thermogenesis, and maintained insulin sensitivity in both liver and fat, without activation of the JNK pathway. Finally, these knockout mice were also found to have reduced expression of inflammatory cytokines, and altered expression of regulatory proteins and enzymes involved in glucose and lipid metabolism. In view of these observations, Chiang and coworkers concluded that IKKε may represent an attractive therapeutic target for obesity, insulin resistance, non-insulin-dependent diabetes mellitus (type 2 diabetes or NIDDM), metabolic syndrome, and other complications associated with these, and other, metabolic diseases and disorders. (Chiang et al.; Cell, 138:961-975, 2009.)

Additionally, TBK1 was implicated as a regulator of the insulin receptor in obese Zucker rats (an art-accepted model of insulin resistance/diabetes), suggesting TBK1 could be involved in mediating insulin resistance (Muñoz et al.; TANK-binding kinase 1 mediates phosphorylation of insulin receptor at serine residue 994: a potential link between inflammation and insulin resistance; J. Endocrinol., 201:185-197, 2009).

In addition to the above-described roles in macrophage activation, antiviral response, and inflammation, the gene encoding IKKε (i.e., IKBKE; Entrez Gene ID: 9641) has been identified as a breast cancer oncogene (Boehm, et al.; Integrative genomic approaches identify IKBKE as a breast cancer oncogene; Cell, 129:1065-1079, 2007). Further, IKKε has been found to directly phosphorylate the tumor suppressor CYLD in vivo, thereby decreasing the activity of CYLD, and leading to transformation and tumorigenesis (Hutti, et al.; Phosphorylation of the tumor suppressor CYLD by the breast cancer oncogene IKKepsilon promotes cell transformation; Mol. Cell, 34:461-472, 2009). In agreement with these observations, it has recently been discovered that overexpression of IKKε is a recurrent event in human ovarian cancer, and that this overexpression could play a role in both tumor progression and the development of cisplatin resistance (Guo, et al.; Deregulation of IKBKE is associated with tumor progression, poor prognosis, and cisplatin resistance in ovarian cancer; Am. J. Pathol., 175:324-333, 2009).

Another role for IKKε has recently been described in triggering an NF-kB antiapoptotic response in response to DNA damage. After genotoxic stress, IKKε translocates to the nucleus and phosphorylates PML to prevent cell death (Renner, et al.; SUMOylation-dependent localization of IKKε in PML nuclear bodies is essential for protection against DNA-damage-triggered cell death; Mol. Cell., 37:503-515, 2010). This newly described activity may contribute to IKKε's role as an oncogene and further support its role as a cancer target.

Additionally, TBK1 (Entrez Gene ID: 29110) has been identified as a proangiogenic gene that is induced under hypoxic conditions and is overexpressed in breast and colon cancers (Korherr, et al.; Identification of proangiogenic genes and pathways by high-throughput functional genomics: TBK1 and the IRF3 pathway; Proc. Natl. Acad. Sci. USA, 103:4240-4245, 2006). In cancer cells, TBK1 was found to restrict initiation of apoptotic programs typically engaged in the context of oncogenic stress (Chien et al.; RalB GTPase-mediated activation of the IκB family kinase TBK1 couples innate immune signaling to tumor cell survival; Cell, 127:157-170, 2006). TBK1 was also recently discovered to exhibit synthetic lethality with oncogenic Ras mutations in cancer cell lines. An RNA interference screen demonstrated potent reduction of cell viability when TBK1 protein was reduced in a Ras mutant background (Barbie, et al.; Systematic RNA interference reveals that oncogenic KRAS-driven cancers require TBK1; Nature, 462:108-112, 2009).

In view of the above, there is a clear need for compounds that selectively inhibit the kinase activities of IKKε, TBK1, or both IKKε and TBK1.

### BRIEF SUMMARY OF THE INVENTION

The present invention provides chemical compounds that selectively inhibit the kinase activities of IKKε, TBK1, or both IKKε and TBK1. Consequently, these compounds may be used in the treatment of inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders.

Specifically, the present invention provides compounds having structures according to Formula I (i.e., compounds according to Formula I):

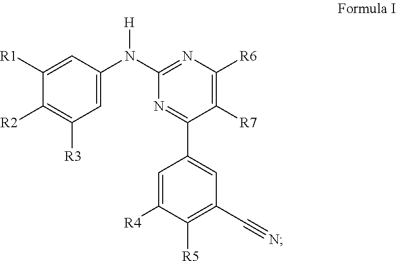

and pharmaceutically acceptable salts thereof;
wherein R1, R2, R3, R4, R5, R6, and R7 are as defined herein below; and,
with the proviso that the compound is NOT:
3-(2-{[3-(hydroxymethyl)-4-(morpholin-4-yl)phenyl]amino}pyrimidin-4-yl)benzonitrile (CAS Registry No. 1187660-52-1);
tert-butyl 1-[5{-[4-(3-cyanophenyl)pyrimidin-2-yl]amino}-2-(morpholin-4-yl)benzyl]-L-prolinate (CAS Registry No. 1187660-08-7);
2-hydroxy-5-(2-{[4-(morpholin-4-yl)phenyl]amino}pyrimidin-4-yl)benzonitrile (CAS Registry No. 1056634-86-6);
2-fluoro-5-{2-[(3,4,5-trimethoxyphenyl)amino]pyrimidin-4-yl}benzonitrile (CAS Registry No. 1056634-82-2);
2-fluoro-5-(2-{[4-(morpholin-4-yl)phenyl]amino}pyrimidin-4-yl)benzonitrile (CAS Registry No. 1056634-78-6);
3-(2-{[4-(morpholin-4-yl)phenyl]amino}pyrimidin-4-yl)benzonitrile (CAS Registry No. 1056634-74-2);
3-{2-[(4-{[4-hydroxy-4-(pyrrolidin-1-ylmethyl)piperidin-1-yl]sulfonyl}phenyl)amino]pyrimidin-4-yl}benzonitrile (CAS Registry No. 1049105-08-9);
3-(2-{[4-(morpholin-4-yl)phenyl]amino}-9H-purin-6-yl)benzonitrile (CAS Registry No. 1042916-08-4);
3-{2-[(4-methoxyphenyl)amino]pyrimidin-4-yl}benzonitrile (CAS Registry No. 902502-38-9);
3-{2-[(4-hydroxyphenyl)amino]pyrimidin-4-yl}benzonitrile (CAS Registry No. 839727-81-0);
3-{2-[(3-hydroxyphenyl)amino]pyrimidin-4-yl}benzonitrile (CAS Registry No. 839727-80-9);
5-{2-[(3,5-dimethylphenyl)amino]pyrimidin-4-yl}-2-ethoxybenzonitrile (CAS Registry No. 691895-41-7);
3-[2-(phenylamino)pyrimidin-4-yl]benzonitrile (CAS Registry No. 663611-44-7); or
3-(2-{[4-(1,1,2,2-tetrafluoroethoxy)phenyl]amino}pyrimidin-4-yl)benzonitrile (CAS Registry No. 170141-17-0).

The compounds of the present invention include the compounds according to Formula I as illustrated herein, as well as their geometric isomers, enantiomers, diastereomers, or racemates thereof. The compounds of the present invention also include the pharmaceutically acceptable salts of such compounds.

As noted above, the present invention provides chemical compounds that selectively inhibit the kinase activities of IKKε, TBK1, or both IKKε and TBK1, and therefore can be used in the treatment of inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders. Thus, the present invention also provides methods for treating inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders, by administering to a patient in need of such treatment a therapeutically effective amount of a compound of the present invention, particularly a compound according to Formula I, or a pharmaceutically acceptable salt thereof.

Also provided is the use of at least one of the compounds according to Formula I for the manufacture of a medicament useful for therapy, including therapy for the treatment of inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders. In addition, the present invention also provides pharmaceutical compositions having at least one compound according to Formula I and one or more pharmaceutically acceptable excipients. Further, methods for the treatment of inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders, by administering to a patient in need of such treatment, a pharmaceutical composition of the invention, are also encompassed.

In addition, the present invention also provides methods for treating or delaying the onset of the symptoms associated with inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders.

These methods comprise administering an effective amount of a compound of the present invention, generally in the form of a pharmaceutical composition or medicament, to an individual having, or at risk of having, inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders.

The compounds according to Formula I may also be used in combination therapies. Thus, combination therapy methods are also provided for treating or delaying the onset of the symptoms associated with inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders. Such methods comprise administering to a patient in need thereof a compound of the present invention and, together or separately, at least one other anti-cancer, anti-inflammation, anti-rheumatoid arthritis, anti-obesity, anti-insulin resistance, anti-metabolic syndrome, anti-type 2 diabetes, anti-SLE, or anti-psoriasis therapy.

For the convenience of combination therapy, the compound of the present invention may be administered together in the same formulation with another agent or therapeutic compound used for treating inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer. Thus, the present invention also provides pharmaceutical compositions or medicaments for combination therapy, comprising an effective amount of at least one compound according to the present invention, and an effective amount of at least one other therapeutic agent or compound, which is different from the compounds according to Formula I.

The foregoing and other advantages and features of the invention, and the manner in which they are accomplished, will become more readily apparent upon consideration of the following detailed description of the invention taken in conjunction with the accompanying examples, which illustrate embodiments of the present invention.

Unless otherwise defined, the technical and scientific terms used herein have the same meaning as commonly understood by one of ordinary skill in the art to which the present invention pertains. Although methods and materials similar or equivalent to those described herein may be used in the practice or testing of the present invention, suitable methods and materials are described below. In case of conflict, the present specification, including definitions, will control. In addition, the materials, methods, and examples are illustrative and not intended to be limiting.

Other features and advantages of the invention will be apparent to one of skill in the art from the following detailed description, and from the claims.

### BRIEF DESCRIPTION OF THE DRAWINGS

FIG. 1 depicts the onset of collagen-induced arthritis as a function of time in mice treated with two dosage strengths of a compound according to Formula 1 or a vehicle-only control.

FIG. 2 depicts the average cumulative severity of collagen-induced arthritis as a function of time in mice treated with two dosage strengths of a compound according to Formula 1 or a vehicle-only control.

FIG. 3 depicts the disease severity score of collagen-induced arthritis for two dosage strengths of a compound according to Formula 1 or a vehicle-only control.

FIG. 4 depicts the loss of average body weight as a function of time in mice with collagen-induced arthritis treated with two dosage strengths of a compound according to Formula 1 or a vehicle-only control.

FIG. 5 shows the production of RANTES by RAW264.7 cells treated with a variety of cytosolic nucleic acid receptor agonists in the presence and absence of a compound according to Formula 1.

FIG. 6 shows the production of interferon beta (IFN-β) by RAW264.7 cells treated with a variety of cytosolic nucleic acid receptor agonists in the presence and absence of a compound according to Formula 1.

FIG. 7 depicts the effects of different concentrations of a compound according to Formula 1 on production of IFN-α2-encoding mRNA by peripheral blood mononuclear cells (PBMCs) isolated from healthy humans in response to induction with a low molecular weight (LMW) and a high molecular weight (HMW) nucleic acid agonist (poly(I:C)).

FIG. 8 depicts the effects of different concentrations of a compound according to Formula 1 on production of IFN-β-encoding mRNA by PBMCs isolated from healthy humans in response to induction with a LMW and a HMW nucleic acid agonist (poly(I:C)).

FIG. 9 depicts the effects of different concentrations of a compound according to Formula 1 on production of BLyS-encoding mRNA by PBMCs isolated from healthy humans in response to induction with a LMW and a HMW nucleic acid agonist (poly(I:C)).

FIG. 10 depicts the effects of different concentrations of a compound according to Formula 1 on production of IFN-α2-encoding mRNA by PBMCs isolated from human SLE patients in response to induction with a LMW nucleic acid agonist (poly(I:C)).

FIG. 11 depicts the effects of different concentrations of a compound according to Formula 1 on production of IFN-β-encoding mRNA by PBMCs isolated from human SLE patients in response to induction with a LMW nucleic acid agonist (poly(I:C)).

FIG. 12 depicts the effects of different concentrations of a compound according to Formula 1 on production of BLyS-encoding mRNA by PBMCs isolated from human SLE patients in response to induction with a LMW nucleic acid agonist (poly(I:C)).

### DETAILED DESCRIPTION OF THE INVENTION

### 1. Definitions

As used herein, the terms “alkyl” or “alkyl group,” as employed herein alone or as part of another group refers to a saturated aliphatic hydrocarbon straight chain group having, unless otherwise specified, 1 to 20 carbon atoms (whenever it appears herein, a numerical range such as “1 to 20” refers to each integer in the given range; e.g., “1 to 20 carbon atoms” means that the alkyl group may consist of 1, 2, 3, 4, 5, 6, 7, 8, 9, 10, 11, 12, 13, 14, 15, 16, 17, 18, 19 or 20 carbon atoms), or a saturated aliphatic hydrocarbon branched chain group having 3, 4, 5, 6, 7, 8, 9, 10, 11, 12, 13, 14, 15, 16, 17, 18, 19 or 20 carbon atoms. An alkyl group may be optionally substituted with one or more substituents as valencies allow (generally one to three substitutents except in the case of halogen substituents, e.g., perchloro). As used herein, a C₁₋₆ alkyl group refers to an alkyl having 1, 2, 3, 4, 5, or 6 carbon atoms (e.g., including methyl, ethyl, propyl, isopropyl, butyl, sec-butyl, tert-butyl, 3-pentyl, and hexyl), which may be optionally substituted.

The term “lower alkyl” as used herein, refers to an alkyl group, as defined above, but containing 1, 2, 3, 4, 5, or 6 carbon atoms (i.e., a C₁₋₆ alkyl group).

The term “alkylene,” or “alkylene group,” as used herein means a saturated aliphatic hydrocarbon straight chain group having 1, 2, 3, 4, 5, 6, 7, 8, 9, 10, 11, 12, 13, 14, 15, 16, 17, 18, 19 or 20 carbon atoms or a saturated aliphatic hydrocarbon branched chain group having 3, 4, 5, 6, 7, 8, 9, 10, 11, 12, 13, 14, 15, 16, 17, 18, 19 or 20 carbon atoms having two connecting points. For example, an “ethylene” group represents the group —CH₂—CH₂—. Alkylene groups may also be optionally substituted with one or more substituents.

The term “alkenyl” as employed herein by itself or as part of another group means a straight chain radical of 2, 3, 4, 5, 6, 7, 8, 9, or 10 carbon atoms or a branched chain radical of 3, 4, 5, 6, 7, 8, 9, or 10 carbon atoms, unless the chain length is limited thereto, including at least one double bond between two of the carbon atoms in the chain. The alkenyl group may be optionally substituted with one or more substituents (generally one to three substitutents except in the case of halogen substituents, e.g., perchloro or perfluoroalkyls). For example, a C₃₋₆ alkenyl group refers to a straight or branched chain radical containing 3, 4, 5 or 6 carbon atoms and having at least one double bond between two of the carbon atoms in the chain (e.g., ethenyl, 1-propenyl, 2-propenyl, 2-methyl-1-propenyl, 1-butenyl and 2-butenyl, which may be optionally substituted).

The term “alkenylene” as used herein means an alkenyl group having two connecting points. For example, “ethenylene” represents the group —CH═CH—. Alkenylene groups may also be optionally substituted with one or more substituents.

The term “alkynyl” as used herein by itself or as part of another group means a straight chain radical of 2, 3, 4, 5, 6, 7, 8, 9, or 10 carbon atoms or branched chain radical of 4, 5, 6, 7, 8, 9, or 10 carbon atoms, unless the chain length is limited thereto, wherein there is at least one triple bond between two of the carbon atoms in the chain. The alkynyl group may be optionally substituted with one or more substituents as valencies allow (generally one to three substitutents except in the case of halogen substituents, e.g., perchloro or perfluoroalkyls). For example, a C₄₋₆ alkynyl group refers to a straight or branched chain radical containing 4, 5, or 6 carbon atoms and having at least one triple bond between two of the carbon atoms in the chain (e.g., ethynyl, 1-propynyl, 1-methyl-2-propynyl, 2-propynyl, 1-butynyl and 2-butynyl), which may be optionally substituted.

The term “alkynylene” as used herein means an alkynyl having two connecting points. For example, “ethynylene” represents the group —C≡C—. Alkynylene groups may also be optionally substituted with one or more substituents.

The term “carbocycle” as used herein by itself or as part of another group means cycloalkyl and non-aromatic partially saturated carbocyclic groups such as cycloalkenyl and cycloalkynyl. A carbocycle may be optionally substituted with one or more substituents so long as the resulting compound is sufficiently stable and suitable for the uses of the present invention.

The term “cycloalkyl” as used herein by itself or as part of another group refers to a fully saturated 3, 4, 5, 6, 7, or 8-membered cyclic hydrocarbon ring (i.e., a cyclic form of an alkyl) alone (“monocyclic cycloalkyl”) or fused to another cycloalkyl, cycloalkynyl, cycloalkenyl, heterocycle, aryl or heteroaryl ring (i.e., sharing an adjacent pair of carbon atoms with such other rings) (“polycyclic cycloalkyl”). Thus, a cycloalkyl may exist as a monocyclic ring, bicyclic ring, or a spiral ring. When a cycloalkyl is referred to as a Cₓ cycloalkyl, this means a cycloalkyl in which the fully saturated cyclic hydrocarbon ring (which may or may not be fused to another ring) has x number of carbon atoms. When a cycloalkyl is recited as a substituent on a chemical entity, it is intended that the cycloalkyl moiety is attached to the entity through a carbon atom within the fully saturated cyclic hydrocarbon ring of the cycloalkyl. In contrast, a substituent on a cycloalkyl can be attached to any carbon atom of the cycloalkyl. A cycloalkyl group may be optionally substituted with one or more substitutents so long as the resulting compound is sufficiently stable and suitable for the uses of the present invention. Examples of cycloalkyl groups include cyclopropyl, cyclobutyl, cyclopentyl, cyclohexyl and cycloheptyl.

The term “cycloalkenyl” as used herein by itself or as part of another group refers to a non-aromatic partially saturated 3, 4, 5, 6, 7, or 8-membered cyclic hydrocarbon ring having at least one double bond therein (i.e., a cyclic form of an alkenyl) alone (“monocyclic cycloalkenyl”) or fused to another cycloalkyl, cycloalkynyl, cycloalkenyl, heterocycle, aryl or heteroaryl ring (i.e., sharing an adjacent pair of carbon atoms with such other rings) (“polycyclic cycloalkenyl”). Thus, a cycloalkenyl may exist as a monocyclic ring, bicyclic ring, polycyclic or a spiral ring. When a cycloalkenyl is referred to as a Cₓ cycloalkenyl, this means a cycloalkenyl in which the non-aromatic partially saturated cyclic hydrocarbon ring (which may or may not be fused to another ring) has x number of carbon atoms. When a cycloalkenyl is recited as a substituent on a chemical entity, it is intended that the cycloalkenyl moiety is attached to the entity through a carbon atom within the non-aromatic partially saturated ring (having a double bond therein) of the cycloalkenyl. In contrast, a substituent on a cycloalkenyl can be attached to any carbon atom of the cycloalkenyl. A cycloalkenyl group may be optionally substituted with one or more substitutents. Examples of cycloalkenyl groups include cyclopentenyl, cycloheptenyl and cyclooctenyl.

The term “heterocycle” (or “heterocyclyl” or “heterocyclic”) as used herein by itself or as part of another group means a saturated or partially saturated 3, 4, 5, 6, or 7-membered non-aromatic cyclic ring formed with carbon atoms and from one to four heteroatoms independently chosen from O, N, and S, wherein the nitrogen and sulfur heteroatoms can be optionally oxidized, and the nitrogen can be optionally quaternized (“monocyclic heterocycle”). The term “heterocycle” also encompasses a group having the non-aromatic heteroatom-containing cyclic ring above fused to another monocyclic cycloalkyl, cycloalkynyl, cycloalkenyl, heterocycle, aryl or heteroaryl ring (i.e., sharing an adjacent pair of atoms with such other rings) (“polycyclic heterocycle”). Thus, a heterocycle may exist as a monocyclic ring, bicyclic ring, polycyclic or a spiral ring. When a heterocycle is recited as a substituent on a chemical entity, it is intended that the heterocycle moiety is attached to the entity through an atom within the saturated or partially saturated ring of the heterocycle. In contrast, a substituent on a heterocycle can be attached to any suitable atom of the heterocycle. In a “saturated heterocycle” the non-aromatic heteroatom-containing cyclic ring described above is fully saturated, whereas a “partially saturated heterocyle” contains one or more double or triple bonds within the non-aromatic heteroatom-containing cyclic ring regardless of the other ring it is fused to. A heterocycle may be optionally substituted with one or more substituents so long as the resulting compound is sufficiently stable and suitable for the uses of the present invention.

Some examples of saturated or partially saturated heterocyclic groups include tetrahydrofuranyl, pyranyl, tetrahydropyranyl, piperidinyl, piperazinyl, pyrrolidinyl, imidazolidinyl, imidazolinyl, indolinyl, isoindolinyl, quinuclidinyl, morpholinyl, isochromanyl, chromanyl, pyrazolidinyl, pyrazolinyl, tetronoyl and tetramoyl groups.

As used herein, “aryl” by itself or as part of another group means an all-carbon aromatic ring with 6 or 8 carbon atoms in the ring (“monocylic aryl”). In addition to monocyclic aromatic rings, the term “aryl” also encompasses a group having the all-carbon aromatic ring above fused to another cycloalkyl, cycloalkynyl, cycloalkenyl, heterocycle, aryl or heteroaryl ring (i.e., sharing an adjacent pair of carbon atoms with such other rings) (“polycyclic aryl”). When an aryl is referred to as a Cₓ aryl, this means an aryl in which the all-carbon aromatic ring (which may or may not be fused to another ring) has x number of carbon atoms. When an aryl is recited as a substituent on a chemical entity, it is intended that the aryl moiety is attached to the entity through an atom within the all-carbon aromatic ring of the aryl. In contrast, a substituent on an aryl can be attached to any suitable atom of the aryl. Examples, without limitation, of aryl groups are phenyl, naphthalenyl and anthracenyl. An aryl may be optionally substituted with one or more substituents so long as the resulting compound is sufficiently stable and suitable for the uses of the present invention.

The term “heteroaryl” as employed herein refers to a stable aromatic ring having 5, 6 or 7 ring atoms with 1, 2, 3 or 4 hetero ring actoms in the ring which are oxygen, nitrogen or sulfur or a combination thereof (“monocylic heteroaryl”). In addition to monocyclic hetero aromatic rings, the term “heteroaryl” also encompasses a group having the monocyclic hetero aromatic ring above fused to another cycloalkyl, cycloalkynyl, cycloalkenyl, heterocycle, aryl or heteroaryl ring (i.e., sharing an adjacent pair of atoms with such other rings) (“polycyclic heteroaryl”). When a heteroaryl is recited as a substituent on a chemical entity, it is intended that the heteroaryl moiety is attached to the entity through an atom within the hetero aromatic ring of the heteroaryl. In contrast, a substituent on a heteroaryl can be attached to any suitable atom of the heteroaryl. A heteroaryl may be optionally substituted with one or more substituents so long as the resulting compound is sufficiently stable and suitable for the uses of the present invention.

Heteroaryl groups include, for example, thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, including without limitation 2H-pyrrolyl, imidazolyl, pyrazolyl, pyridyl (pyridinyl), including without limitation 2-pyridyl, 3-pyridyl, and 4-pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, β-carbolinyl, phenanthridinyl, acrindinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-amino-isocoumarin, pyrido[1,2-a]pyrimidin-4-one, pyrazolo[1,5-a]pyrimidinyl, including without limitation pyrazolo[1,5-a]pyrimidin-3-yl, 1,2-benzoisoxazol-3-yl, benzimidazolyl, 2-oxindolyl and 2-oxobenzimidazolyl. Where the heteroaryl group contains a nitrogen atom in a ring, such nitrogen atom may be in the form of an N-oxide, e.g., a pyridyl N-oxide, pyrazinyl N-oxide and pyrimidinyl N-oxide.

As used herein, the term “halo” refers to fluoro, chloro, bromo, or iodo substitutents.

As used herein, the term “hydro” refers to a bound hydrogen (i.e., an —H group).

As used herein, the term “hydroxyl” refers to an —OH group.

As used herein, the term “alkoxy” refers to an —O-(alkyl). Lower alkoxy refers to —O-(lower alkyl) groups.

As used herein, the term “alkenyloxy” refers to an —O-(alkenyl).

As used herein, the term “alkynyloxy” refers to an —O-(alkynyl).

As used herein, the term “cycloalkyloxy” refers to an —O-cycloakyl group.

As used herein, the term “heterocycloxy” refers to an —O-heterocycle group.

As used herein, the term “mercapto” group refers to an —SH group.

The term “alkylthio” group refers to an —S-alkyl group.

The term “arylthio” group refers to an —S-aryl group.

The term “arylalkyl” is used herein to mean an alkyl group, as defined above, substituted with an aryl group, as defined above. Examples of arylalkyl groups include benzyl, phenethyl and naphthylmethyl, etc. An arylalkyl group may be optionally substituted with one or more substituents so long as the resulting compound is sufficiently stable and suitable for the uses of the present invention.

The term “heteroarylalkyl” is used herein to mean an alkyl group, as defined above, substituted with a heteroaryl group, as defined above. A heteroarylalkyl may be optionally substituted with one or more substituents so long as the resulting compound is sufficiently stable and suitable for the uses of the present invention.

The term “arylalkynyl” is used herein to mean any of the above-defined alkynyl groups substituted with any of the above-defined aryl groups.

The term “heteroarylalkenyl” is used herein to mean any of the above-defined alkenyl groups substituted with any of the above-defined heteroaryl groups.

The term “aryloxy” is used herein to mean aryl-O— or —O-aryl wherein aryl is as defined above. Aryloxy groups include phenoxy and 4-methylphenoxy.

The term “heteroaryloxy” is used herein to mean heteroaryl-O— or —O-heteroaryl wherein heteroaryl is as defined above.

The term “arylalkoxy” is used herein to mean an alkoxy group substituted with an aryl group as defined above. Arylalkoxy groups include benzyloxy and phenethyloxy.

“Heteroarylalkoxy” is used herein to mean any of the above-defined alkoxy groups substituted with any of the above-defined heteroaryl groups.

“Haloalkyl” means an alkyl group that is substituted with one or more fluorine, chlorine, bromine or iodine atoms. Haloalkyl groups include, for example, fluoromethyl, difluoromethyl, trifluoromethyl, pentafluoroethyl, 1,1-difluoroethyl, chloromethyl, chlorofluoromethyl and trichloromethyl groups.

As used herein, the term “oxo” refers to an oxygen atom double bonded to another atom (i.e., “═O”).

As used herein, the term “carbonyl” group refers to a —C(═O)R″ group, where R″ is chosen from hydro, alkyl, cycloalkyl, aryl, heteroaryl (bonded through a ring carbon) and heterocyclic (bonded through a ring carbon), as defined herein.

As used herein, the term “aldehyde” group refers to a carbonyl group where R″ is hydro.

As used herein, the term “cycloketone” refer to a cycloalkyl group in which one of the carbon atoms which form the ring has a “═O” bonded to it; i.e. one of the ring carbon atoms is a —C(═O)— group.

As used herein, the term “thiocarbonyl” group refers to a —C(═S)R″ group, with R″ as defined herein. “Alkylthiocarbonyl” refers to an alkyl-C(═S)— group.

“Alkanoyl,” as used herein, refers to an alkyl-C(═O)— group.

As used herein the term “acetyl” group refers to a —C(═O)CH₃ group.

The term “heterocycloketone,” as used herein refers to a heterocycle group in which one of the carbon atoms which form the ring has an oxygen double-bonded to it—i.e., one of the ring carbon atoms is a —C(═O)— group.

As used herein the term “O-carboxy” group refers to a R″C(═O)O— group, where R″ is as defined herein.

The term “C-carboxy” group, as used herein, refers to a —C(═O)OR″ groups where R″ is as defined herein.

As used herein, the term “carboxylic acid” refers to a C-carboxy group in which R″ is hydro. In other words, the term “carboxylic acid” refers to —COOH.

As used herein, the term “ester” is a C-carboxy group, as defined herein, wherein R″ is as defined above, except that it is not hydro. Example ester groups include, methyl ester, ethyl ester, propyl ester, and lower alkyl ester).

As used herein, the term “C-carboxy salt” refers to a —C(═O)O⁻M⁺ group wherein M⁺ is chosen from lithium, sodium, magnesium, calcium, potassium, barium, iron, zinc and quaternary ammonium.

The term “carboxyalkyl,” as used herein, refers to —C₁₋₆ alkylene-C(═O)OR″ (that is, a C₁₋₆ alkyl group connected to the core structure wherein the alkyl group is substituted with —C(═O)OR″ with R″ being defined herein). Examples of carboxyalkyl include, but are not limited to, —CH₂COOH, —(CH₂)₂COOH, —(CH₂)₃COOH, —(CH₂)₄COOH, and —(CH₂)₅COOH.

“Carboxyalkenyl” refers to -alkenylene-C(═O)OR″ with R″ being defined herein.

The term “carboxyalkyl salt” refers to a —(CH₂)ᵣC(═O)O⁻M⁺ wherein M⁺ is chosen from lithium, sodium, potassium, calcium, magnesium, barium, iron, zinc and quaternary ammonium, wherein r is 1, 2, 3, 4, 5, or 6.

The term “carboxyalkoxy” refers to —O—(CH₂)ᵣC(═O)OR″ wherein r is 1, 2, 3, 4, 5, or 6, and R″ is as defined herein.

“Cₓ carboxyalkanoyl” means a carbonyl group (—C(═O)—) attached to an alkyl or cycloalkylalkyl group that is substituted with a carboxylic acid or carboxyalkyl group, wherein the total number of carbon atom is x (an integer of 2 or greater).

“Cₓ carboxyalkenoyl” means a carbonyl group (—C(═O)—) attached to an alkenyl or alkyl or cycloalkylalkyl group that is substituted with a carboxylic acid or carboxyalkyl or carboxyalkenyl group, wherein at least one double bond (—CH═CH—) is present and wherein the total number of carbon atom is x (an integer of 2 or greater).

“Carboxyalkoxyalkanoyl” means refers to R″OC(═O)—C₁₋₆ alkylene-O—C₁₋₆ alkylene-C(═O)—, R″ is as defined herein.

As used herein, the term “heterocycloyl”, by itself or as part of another group, means a radical of formula heterocycle-C(═O)—.

“Amino” refers to an —NR^{x}R^{y} group, with R^{x} and R^{y} as defined herein.

“Alkylamino,” as used herein, means an amino group with at least one alkyl substituent.

“Aminoalkyl” means an alkyl group connected to the core structure of a molecule and having at least one amino substituent.

“Quaternary ammonium” refers to a —⁺N(R^{x})(R^{y})(R^{z}) group wherein R^{x}, R^{y}, and R^{z} are as defined herein.

The term “nitro” refers to a —NO₂ group.

As used herein the term “O-carbamyl” refers to a —OC(═O)N(R^{x})(R^{y}) group with R^{x} and R^{y} as defined herein.

The term “N-carbamyl,” as used herein, refers to a R^{y}OC(═O)N(R^{x})— group, with R^{x} and R^{y} as defined herein.

As used herein the term “O-thiocarbamyl” refers to a —OC(═S)N(R^{x})(R^{y}) group with R^{x} and R^{y} as defined herein.

The term “N-thiocarbamyl,” as used herein, refers to a R^{x}OC(═S)NR^{y}— group, with R^{x} and R^{y} as defined herein.

As used herein the term “C-amido” refers to a —C(═O)N(R^{x})(R^{y}) group with R^{x} and R^{y} as defined herein.

“N-amido,” as used herein, refers to a R^{x}C(═O)N(R^{y})— group with R^{x} and R^{y} as defined herein.

“Carbamoylamino” or “carbamide linker” are used alternatively herein to refer to a R″N(R^{y})C(═O)N(R^{x})— group with R^{x}, R^{y} and R″ as defined herein.

“Aminothiocarbonyl” refers to a —C(═S)N(R^{x})(R^{y}) group with R^{x} and R^{y} as defined herein.

“Hydroxyaminocarbonyl” means a —C(═O)N(R^{x})(OH) group with R^{x} as defined herein.

“Alkoxyaminocarbonyl” means a —C(═O)N(R^{x})(alkoxy) group with R^{x} as defined herein.

The terms “cyano,” “cyanyl,” and “nitrile” group, as used herein, refer to a —C≡N group.

The term “cyanato” refers to a —CNO group.

The term “isocyanato” refers to a —NCO group.

The term “thiocyanato” refers to a —CNS group.

The term “isothiocyanato” refers to a —NCS group.

The term “sulfinyl” refers to a —S(═O)R″ group, where R″ is as defined herein.

The term “sulfonyl” refers to a —S(═O)₂R″ group, where R″ is as defined herein.

The term “sulfonamide” or “sulfamoyl” are used interchangeably herein to refer to an —N(R^{x})—S(═O)₂R″ group, with R″ and R^{x} as defined herein.

“Aminosulfonyl” means (R^{x})(R^{y})N—S(═O)₂— with R^{x} and R^{y} as defined herein.

“Aminosulfonyloxy” means a (R^{x})(R^{y})N—S(═O)₂—O— group with R^{x} and R^{y} as defined herein.

“Sulfonamidecarbonyl” means R″—S(═O)₂—N(R^{x})—C(═O)— with R″ and R^{x} as defined herein.

“Alkanoylaminosulfonyl” refers to an alkyl-C(═O)—N(R^{x})—S(═O)₂— group with R^{x} as defined herein.

The term “trihalomethylsulfonyl” refers to a X₃CS(═O)₂— group with X being halo.

The term “trihalomethylsulfonamide” refers to a X₃CS(═O)₂N(R^{x})— group with X being halo and R^{x} as defined herein.

R″ is chosen from hydro, alkyl, cycloalkyl, aryl, heteroaryl and heterocycle, each being optionally substituted.

R^{x}, R^{y}, and R^{z} are independently chosen from hydro and optionally substituted alkyl.

The term “methylenedioxy” refers to a —OCH₂O— group wherein the oxygen atoms are bonded to adjacent ring carbon atoms.

The term “ethylenedioxy” refers to a —OCH₂CH₂O— group wherein the oxygen atoms are bonded to adjacent ring carbon atoms.

The term “bioisostere”, as used herein, generally refers to compounds or moieties that have chemical and physical properties producing broadly similar biological properties.

Examples of carboxylic acid bioisosteres include, but are not limited to, carboxyalkyl, carboxylic acid ester, tetrazole, oxadiazole, isoxazole, hydroxythiadiazole, thiazolidinedione, oxazolidinedione, sulfonamide, aminosulfonyl, sulfonamidecarbonyl, C-amido, sulfonylcarboxamide, phosphonic acid, phosphonamide, phosphinic acid, sulfonic acid, alkanoylaminosufonyl, mercaptoazole, trifluoromethylcarbonyl, and cyanamide.

Unless specifically stated otherwise or indicated by a bond symbol (dash, double dash, or triple dash, etc.), the point at which a recited substituent group connects to the remainder of the molecule will be via the right-most stated moiety. Further, the names of chemical moieties, as defined above, can simply be linked together to identify more complex substituent groups. In such instances, the point at which the recited complex substituent is connected to the remainder of the molecule will be through the right-most stated moiety. Thus, for example, a “hydroxyalkyl” group is connected to the remainder of the molecule through the alkyl moiety while the hydroxyl is a substituent on the alkyl. Similarly, for example, a “heterocyclealkyl” group is connected to the remainder of the molecule through the alkyl moiety while the heterocycle is a substituent on the alkyl.

In most instances names for the compounds disclosed were generated in accordance with International Union of Pure and Applied Chemistry (IUPAC) conventions using Advanced Chemistry Development, Inc., (ACD/Labs) (Toronto, Ontario, Canada) ACD/Name IUPAC nomenclature software release 12.00, version 12.01. In some cases, however, names for compounds and synthetic intermediates were generated using the IUPAC naming feature supplied with either the Symyx® Draw package, version 3.2 or 3.3, available from Symyx Technologies, Inc. (Santa Clara, Calif.), or the Autonom 2000 plug-in for the Isis™/Draw 2.5 SP1 chemical drawing program, formerly available from MDL Information Systems, a division of Symyx Technologies, Inc. (Santa Clara, Calif.). In all cases, if there is a conflict between a name and a structure when a structure is provided along with a name, the structure is to be taken as ultimately defining the compound being described.

### 2. Compounds of the Present Invention

The present invention provides chemical compounds that selectively inhibit the kinase activities of IKKε and/or TBK1. Consequently, these compounds may be used in the treatment of inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders.

Specifically, the present invention provides compounds having structures according to Formula I (i.e., compounds according to Formula I):

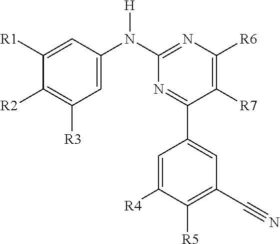

### Formula I;

and pharmaceutically acceptable salts thereof,

wherein R1, R2, R3, and R5 are independently chosen from the following groups:
alkyl, alkylene, alkenyl, alkenylene, alkynyl, carbocycle, cycloalkyl, cycloalkenyl, heterocycle, aryl, heteroaryl, halo, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, cycloalkylthio, arylalkyl, heteroarylalkyl, heteroarylalkenyl, arylalkynyl, haloalkyl, aldehyde, thiocarbonyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, aminothiocarbonyl, hydroxyaminocarbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide,
wherein any of the foregoing groups are optionally substituted at least once with alkyl, alkylene, alkenyl, alkenylene, alkynyl, carbocycle, cycloalkyl, cycloalkenyl, heterocycle, aryl, heteroaryl, halo, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, cycloalkylthio, arylalkyl, heteroarylalkyl, heteroarylalkenyl, arylalkynyl, haloalkyl, aldehyde, thiocarbonyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, aminothiocarbonyl, hydroxyaminocarbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide,
with the proviso that R2 is not heteroaryl; or,
R2 and either R1 or R3, together with the carbon atoms to which they are bound, form an optionally-substituted cycloalkyl, heterocycle, aryl, or heteroaryl;

wherein R4 is independently chosen hydro, halo, and an optionally-substituted group chosen from lower alkyl, haloalkyl, alkoxy, arylalkoxy, heteroarylalkoxy, and heterocycloalkoxy;

wherein R6 and R7 are independently chosen from hydro, halo, and lower alkyl; or

R6, taken together with R7 and the carbon atoms to which they are attached, form a 5 to 6 membered aryl or heteroaryl ring (e.g., imidazole); and,

with the proviso that the compound is NOT:
3-(2-{[3-(hydroxymethyl)-4-(morpholin-4-yl)phenyl]amino}pyrimidin-4-yl)benzonitrile (CAS Registry No. 1187660-52-1);
tert-butyl 1-[5-{[4-(3-cyanophenyl)pyrimidin-2-yl]amino}-2-(morpholin-4-yl)benzyl]-L-prolinate (CAS Registry No. 1187660-08-7);
2-hydroxy-5-(2-{[4-(morpholin-4-yl)phenyl]amino}pyrimidin-4-yl)benzonitrile (CAS Registry No. 1056634-86-6);
2-fluoro-5-{2-[(3,4,5-trimethoxyphenyl)amino]pyrimidin-4-yl}benzonitrile (CAS Registry No. 1056634-82-2);
2-fluoro-5-(2-{[4-(morpholin-4-yl)phenyl]amino}pyrimidin-4-yl)benzonitrile (CAS Registry No. 1056634-78-6);
3-(2-{[4-(morpholin-4-yl)phenyl]amino}pyrimidin-4-yl)benzonitrile (CAS Registry No. 1056634-74-2);
3-{2-[(4-{[4-hydroxy-4-(pyrrolidin-1-ylmethyl)piperidin-1-yl]sulfonyl}phenyl)amino]pyrimidin-4-yl}benzonitrile (CAS Registry No. 1049105-08-9);
3-(2-{[4-(morpholin-4-yl)phenyl]amino}-9H-purin-6-yl)benzonitrile (CAS Registry No. 1042916-08-4);
3-{2-[(4-methoxyphenyl)amino]pyrimidin-4-yl}benzonitrile (CAS Registry No. 902502-38-9);
3-{2-[(4-hydroxyphenyl)amino]pyrimidin-4-yl}benzonitrile (CAS Registry No. 839727-81-0);
3-{2-[(3-hydroxyphenyl)amino]pyrimidin-4-yl}benzonitrile (CAS Registry No. 839727-80-9);
5-{2-[(3,5-dimethylphenyl)amino]pyrimidin-4-yl}-2-ethoxybenzonitrile (CAS Registry No. 691895-41-7);
3-[2-(phenylamino)pyrimidin-4-yl]benzonitrile (CAS Registry No. 663611-44-7); or
3-(2-{[4-(1,1,2,2-tetrafluoroethoxy)phenyl]amino}pyrimidin-4-yl)benzonitrile (CAS Registry No. 170141-17-0).

In particular embodiments of the compounds according to Formula I, R1, R2, R3, and R5 are independently chosen from:
hydro, halo, hydroxyl, mercapto, —NH₂, and carboxylic acid; or
an optionally-substituted substituent group chosen from alkyl, alkylthio, cycloalkylthio, haloalkyl, alkoxy, C-carboxy, amino, alkylamino, aminoalkyl, C-amido, N-amido, aminosulfonyl, sulfonamide, cycloalkyl, heterocycle, heterocycloxy, heteroaryloxy, heteroarylalkoxy, heterocyclealkyl, and arylalkoxy.

In particular embodiments of the compounds according to Formula I, R1, R2, and R3 are independently chosen from:

hydro, halo, hydroxyl, hydroxyalkyl, —NH₂, and carboxylic acid; or

an optionally-substituted substituent group chosen from alkyl, haloalkyl, alkoxy, C-carboxy, amino, C-amido, N-amido, aminosulfonyl, sulfonamide, cycloalkyl, heterocycle, heterocycloxy, heteroaryloxy, heteroarylalkoxy, heterocyclealkyl, and arylalkoxy; or R1, R2, and R3 are independently chosen from the following groups:

(1) (Ra)-(CH₂)ₙ—O—, wherein

n=0, 1, 2, 3 or 4,

Ra is an optionally-substituted substituent group chosen from amino, C-amido, alkyl, hydroxyalkyl, alkoxy, aminoalkoxy, aryl, heterocycle, heterocycloyl, heterocycloalkoxy, heterocyclosulfonyl, heterocyclosulfamoylalkoxy, aminosulfamoylalkoxy, and sulfamoylalkoxy (e.g., any heterocyclo moiety can be further substituted with exemplary groups such as lower alkyl and alkanoyl);

(2) (Rb)(Rc)N—(CH₂)ₙ—, wherein

n=0, 1, 2, 3 or 4,

Rb is chosen from hydro or lower alkyl, or an optionally-substituted substituent group chosen from alkyl, cycloalkyl, alkoxy, aminoalkyl, C-amido, C-amidoalkyl, C-carboxy, heterocycle, heterocycloalkyl, sulfamoyl, alkoxyalkyl, hydroxyalkyl, C-carboxyalkyl, and amino, wherein examples of further optional substituents of each of the foregoing groups include lower alkyl and sulfamoyl;

Rc is chosen from hydro or lower alkyl, or

Rb together with Rc form a 4, 5, 6, or 7-membered optionally-substituted substituent group chosen from heterocycle or heteroaryl, (e.g., wherein the heterocycle or heteroaryl is substituted at least once with hydroxyl, lower alkyl, hydroxyalkyl, sulfonyl, oxo, C-amido, alkoxy, alkoxyalkoxy, alkoxyalkyl, amino, aminoalkyl, or a second optionally-substituted heterocyclic group);

(3) (Rd)(Re)N—C(═O)—(CH₂)ₙ—, wherein

n=0, 1, 2, 3 or 4,

Rd is chosen from hydro, or an optionally-substituted substituent group chosen from aminoalkyl, cycloalkyl, heterocycle, heterocyclealkyl, and heteroarylalkyl;

Re is chosen from hydro or lower alkyl, or

Rd together with Re form a 4, 5, 6, or 7-membered optionally-substituted heterocycle, (e.g., wherein the heterocycle is substituted with lower alkyl, a second optionally-substituted heterocyclic group, or an aminoalkyl group);

(4) (Rf)-C(═O)—N(Rg)-(CH₂)ₙ—, wherein

n=0, 1, 2, 3 or 4,

Rf is chosen from an optionally-substituted substituent group chosen from alkyl, hydroxyalkyl, cycloalkyl, alkoxy, alkoxyalkyl, alkoxyalkoxy, alkoxyalkoxyalkyl, alkylthioalkyl, and heteroaryl, wherein examples of further optional substituents of each of the foregoing groups include lower alkyl and amino; and

Rg is chosen from hydro or lower alkyl;

(5) (Rh)(Ri)N—C(═O)—N(Rj)-(CH₂)ₙ—, wherein

n=0, 1, 2, 3 or 4,

Rh is chosen from an optionally-substituted substituent group chosen from alkyl, cycloalkyl, hydroxyalkyl, alkoxyalkyl, aryl, aminoalkyl, N-amidoalkyl, heterocycle and heteroaryl, wherein examples of further optional substituents of each of the foregoing groups include lower alkyl, alkanoyl, hydroxyl, amino, and alkoxy;

Ri is chosen from hydro or lower alkyl, or

Rh together with Ri form a 4, 5, 6, or 7-membered optionally-substituted heterocycle; and

Rj is chosen from hydro or lower alkyl; or

(6) (Rk)(Rkk)-N—S(═O)₂—(CH₂)ₙ—, wherein

n=0, 1, 2, 3 or 4,

Rk is chosen from hydro or an optionally-substituted substituent group chosen from alkyl, aminoalkyl, hydroxyalkyl, alkanoyl, heteroaryl, heterocycle, heterocyclealkyl, and heteroarylalkyl, wherein examples of further optional substituents of each of the foregoing groups include lower alkyl;

Rkk is chosen from hydro or lower alkyl, or

Rk together with Rkk form a 4, 5, 6, or 7-membered optionally-substituted heterocycle (e.g., wherein the heterocycle is substituted with lower alkyl, amino, and hydroxyalkyl).

In particular embodiments of the compounds according to Formula I,
R4 is chosen from hydro, halo, optionally-substituted alkoxy, and optionally-substituted arylalkoxy.

In particular embodiments of the compounds according to Formula I,

R5 is chosen from

hydro, halo, hydroxyl, mercapto, —NH₂, and carboxylic acid; or

an optionally-substituted substituent group chosen from amino, alkylamino, N-amido, C-amido, C-carboxy, alkyl, alkoxy, cycloalkyl, cycloalkylthio, alkylthio, and heterocycle; or

R5 is chosen from the following groups:

(1) (Rm)-(CH₂)ₙ—O—, wherein

n=0, 1, 2, 3 or 4,

Rm is chosen from hydro or hydroxyl, or an optionally-substituted substituent group chosen from alkyl, hydroxyalkyl, amino, cycloalkyl, C-amido, C-carboxy, aryl, heterocycle, heterocycloyl, and heteroaryl, or

Rm is chosen from one of the following substituted secondary linking groups:

(1a) (Rn)-SO₂—NH—, wherein
Rn is an optionally-substituted alkyl;

(1b) (Ro)-C(═O)—NH—, wherein
Ro is chosen from hydro, or an optionally-substituted substituent group chosen from hydroxyalkyl, alkyl, alkoxy and amino;

(1c) (Rp)-NH—C(═O)—NH—, wherein
Rp is an optionally-substituted alkyl;
   (2) (Rq)-3, 4, 5, or 6 carbon branched alkyl-O—, wherein

Rq is chosen from hydroxyl, carboxylic acid, methyl ester, or an optionally-substituted substituent group chosen from C-carboxy or C-amido;

(3) (Rr)-SO₂—NH—, wherein Rr is an optionally-substituted substituent group chosen from alkyl or haloalkyl;
(4) (Rs)-(CH₂)ₙ—NH—, wherein:

n=0, 1, 2, 3 or 4;

Rs is chosen from an optionally substituted substituent group chosen from akyl, sulfonyl, heterocycle, and heteroaryl;

(5) (Rt)-O—C(═O)—NH—, wherein

Rt is an optionally-substituted alkyl;

(6) (Ru)(Rv)N—C(═O)—NH—, wherein

Ru is chosen from an optionally-substituted substituent group chosen from alkyl, cycloalkyl and heterocycle;

Rv is chosen from hydro or an optionally-substituted alkyl; or

Ru together with Rv form a 4, 5, 6, or 7-membered optionally-substituted heterocycle;

(7) (Rw)-C(═O)—NH—, wherein

Rw is chosen from an optionally-substituted substituent group chosen from alkyl, alkoxy, hydroxyalkyl, aminoalkyl,O-carboxy, haloalkyl, cycloalkyl, aryl, arylalkyl, heterocycle, and heteroaryl;

(8) (Rx)(Ry)N—, wherein

Rx and Ry are independently chosen from hydro, alkyl and sulfonyl, or

Rx together with Ry form a 4, 5, 6, or 7-membered optionally-substituted heterocycle (e.g., wherein the heterocycle is substituted with lower alkyl, a second optionally-substituted heterocyclic group, or an amino group);

(9) (Rz)-(heterocyclic linker)-(CH₂)ₙ—O—, wherein

n=0, 1, 2, 3 or 4, and

the “heterocyclic linker” is chosen from diradicals of the heterocycles azetidine, pyrrolidine, and piperidine, with Rz being attached directly to a heteroatom in the heterocycle; and

Rz is chosen from an optionally-substituted substituent group chosen from alkyl, alkoxy, aldehyde, C-carboxy, C-amido, alkanoyl, haloalkanoyl, aminoalkanoyl, alkylaminoalkanoyl, O-carboxyalkanoyl, alkoxyalkanoyl, hydroxyalkanoyl, cycloalkylalkanoyl, heterocycloalkanoyl, heterocycloyl, heteroarylalkonyl, sulfonyl, and aminosulfonyl.

In particular embodiments of the compounds according to Formula I, R6 and R7 are independently chosen from hydro, halo, and lower alkyl; or R6, taken together with R7, form a 5 to 6 membered aryl or heteroaryl ring (e.g., imidazole).

In particular embodiments of the compounds according to Formula I, wherein the substituent R5 is (Rz)-(heterocyclic linker)-(CH₂)ₙ—O—, the heterocyclic linker and orientation of the linking bonds is chosen from:

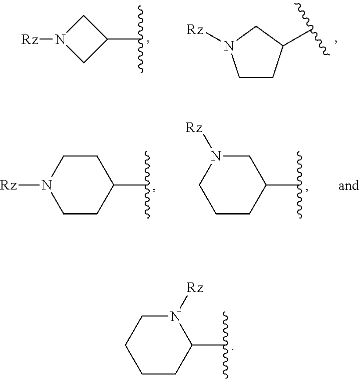

In particular embodiments of the compounds according to Formula I, R1 and R3 are independently chosen from:

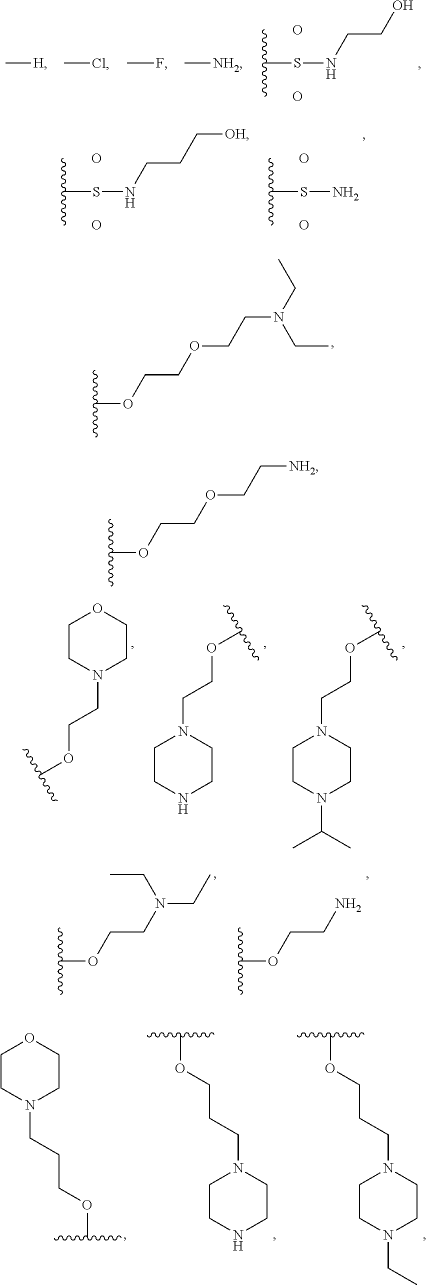

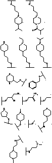

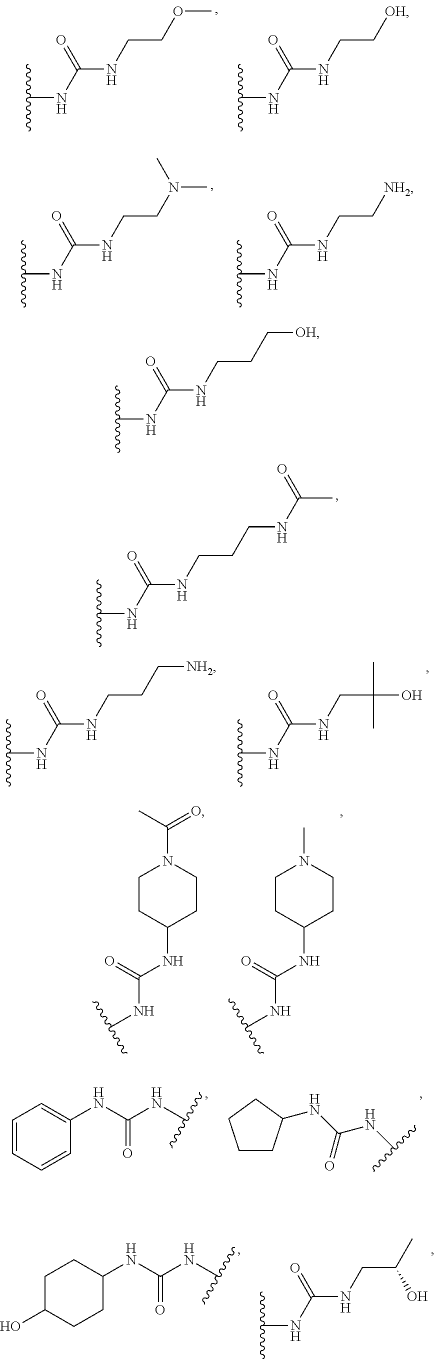

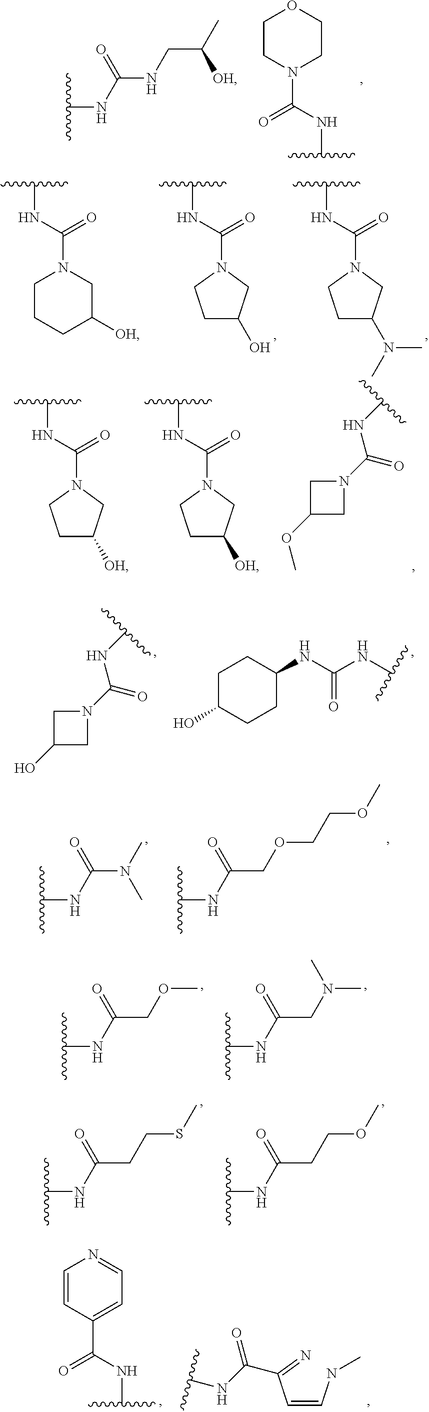

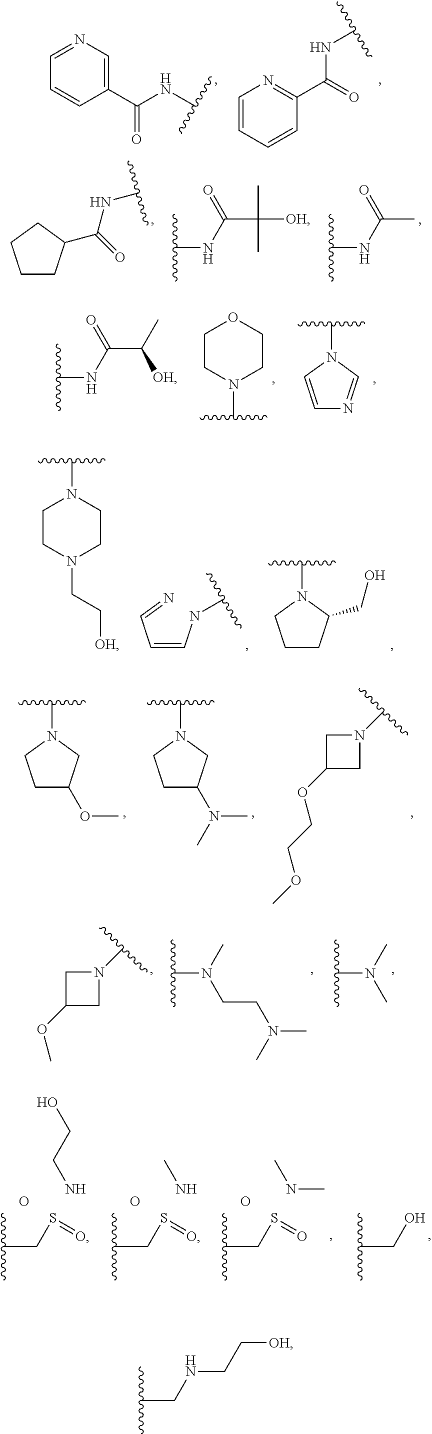

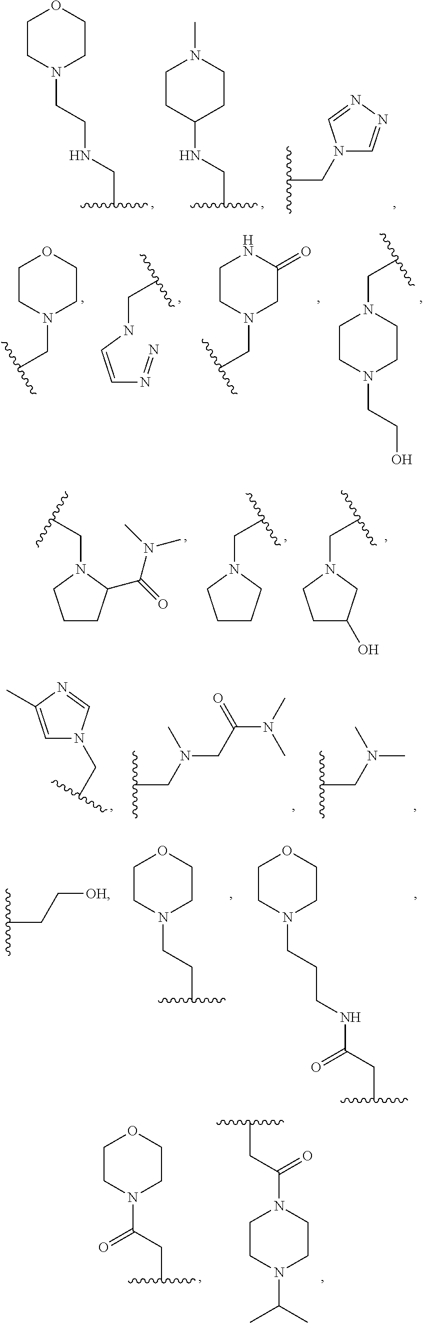

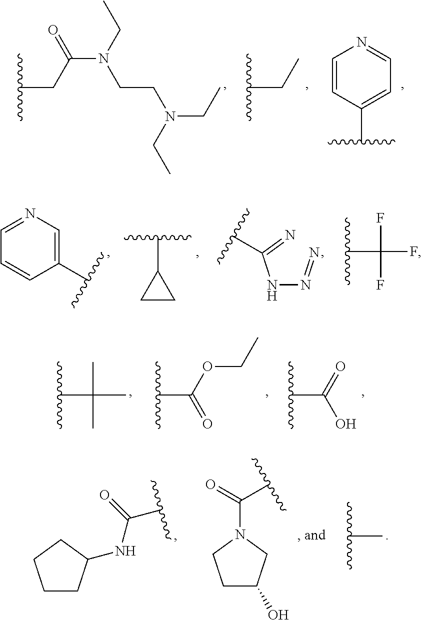

In particular embodiments of the compounds according to Formula I, R2 is chosen from:

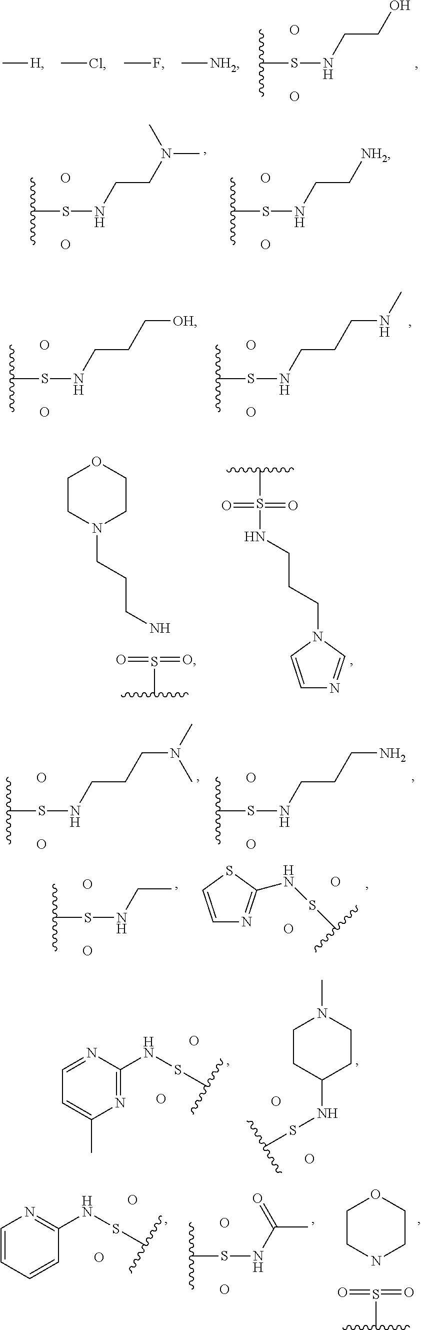

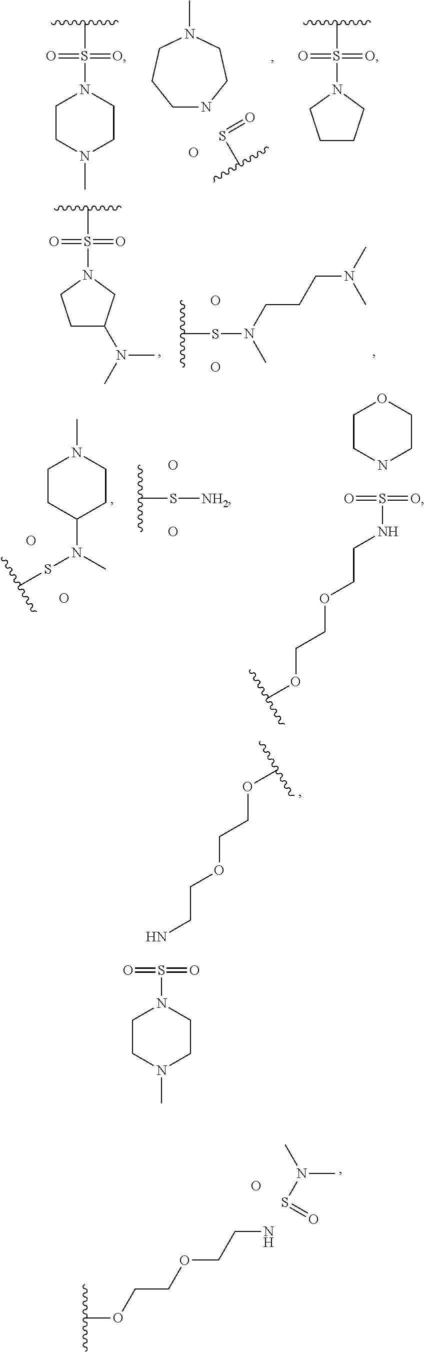

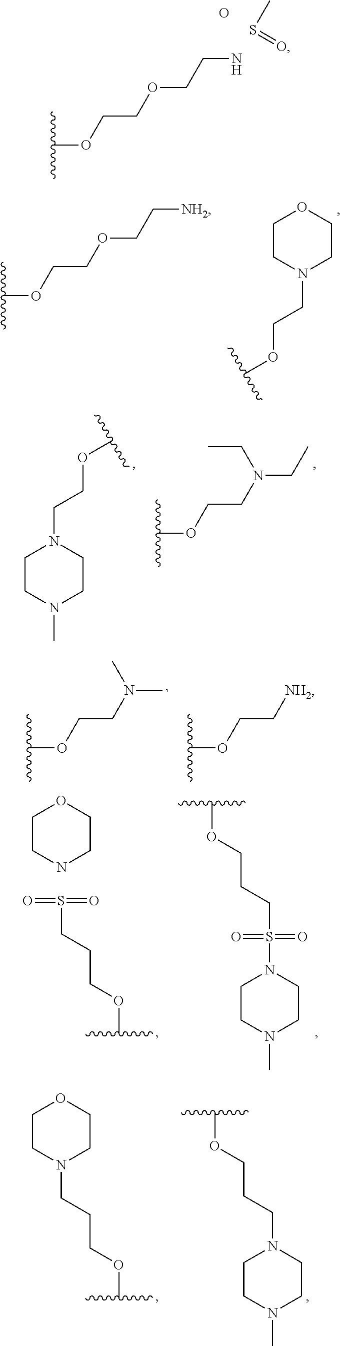

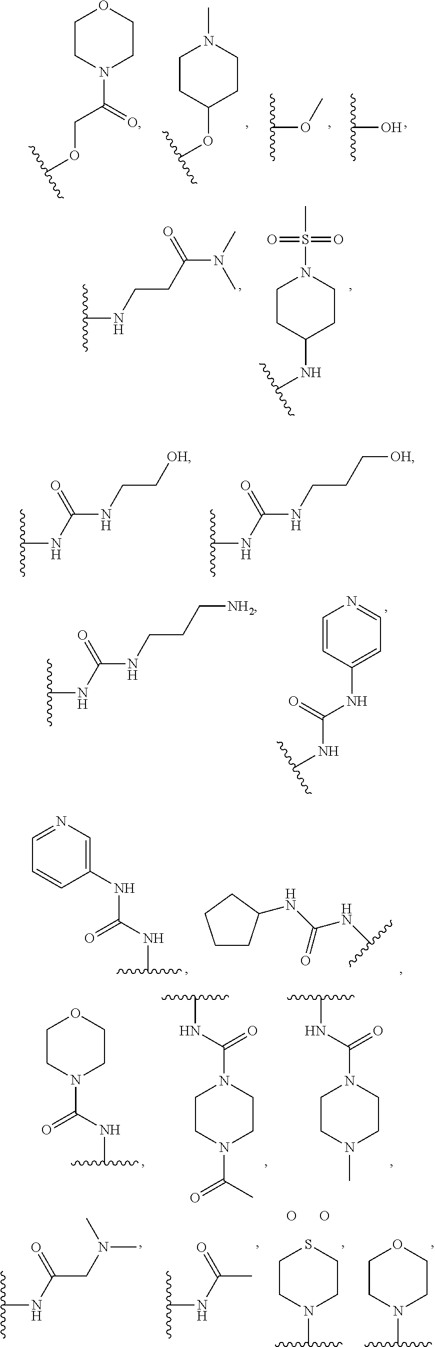

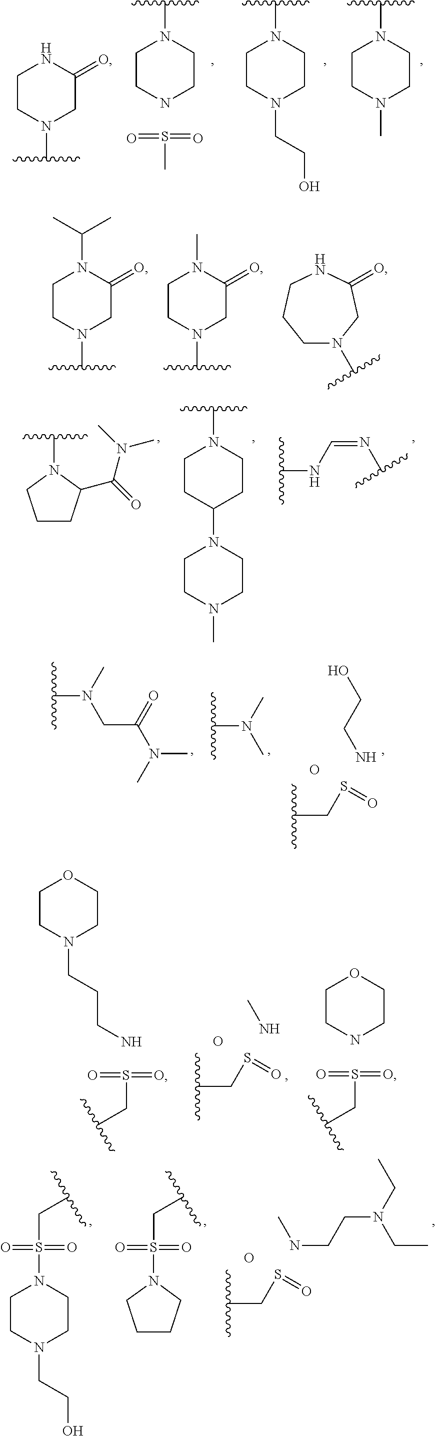

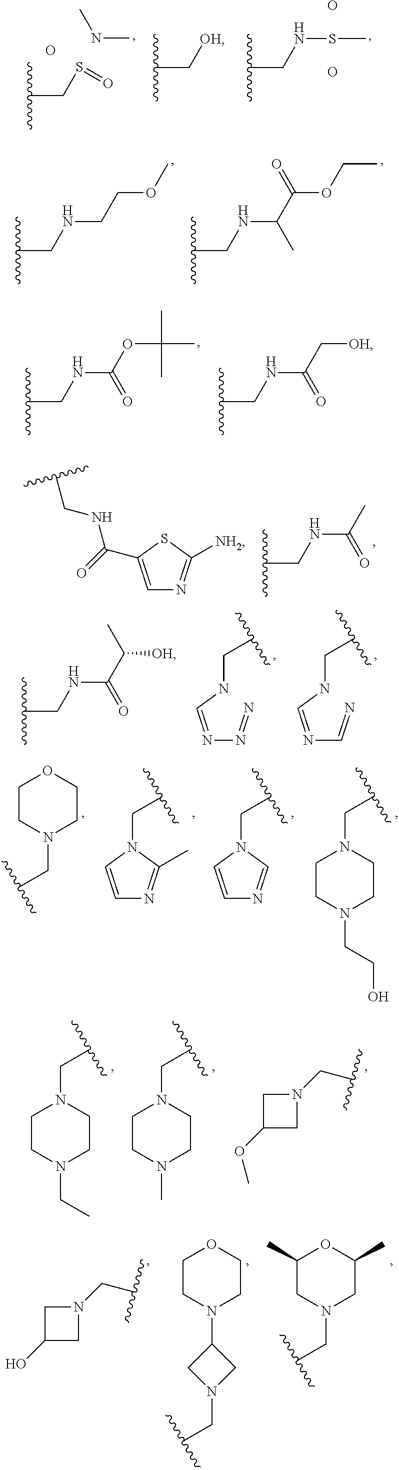

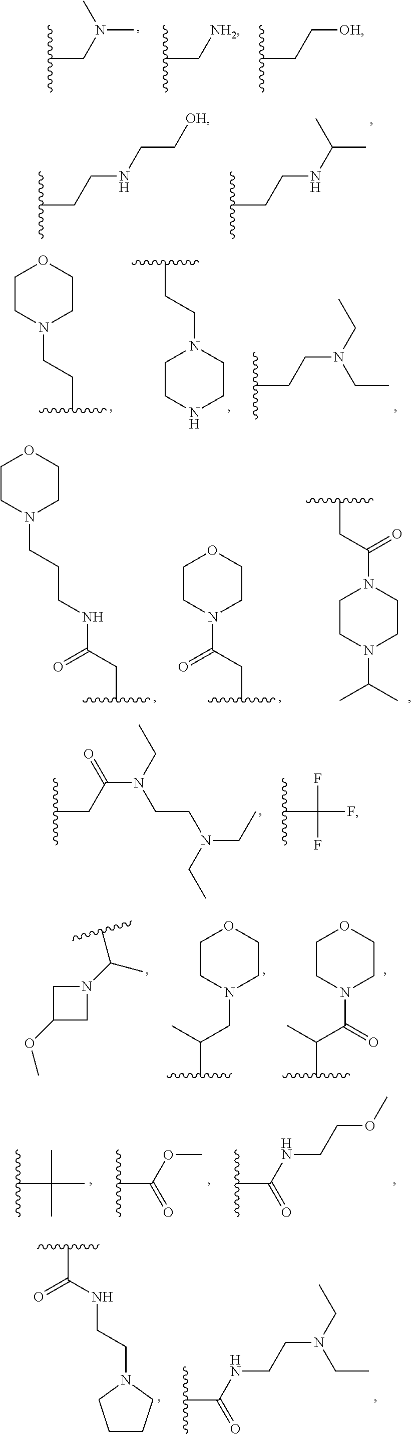

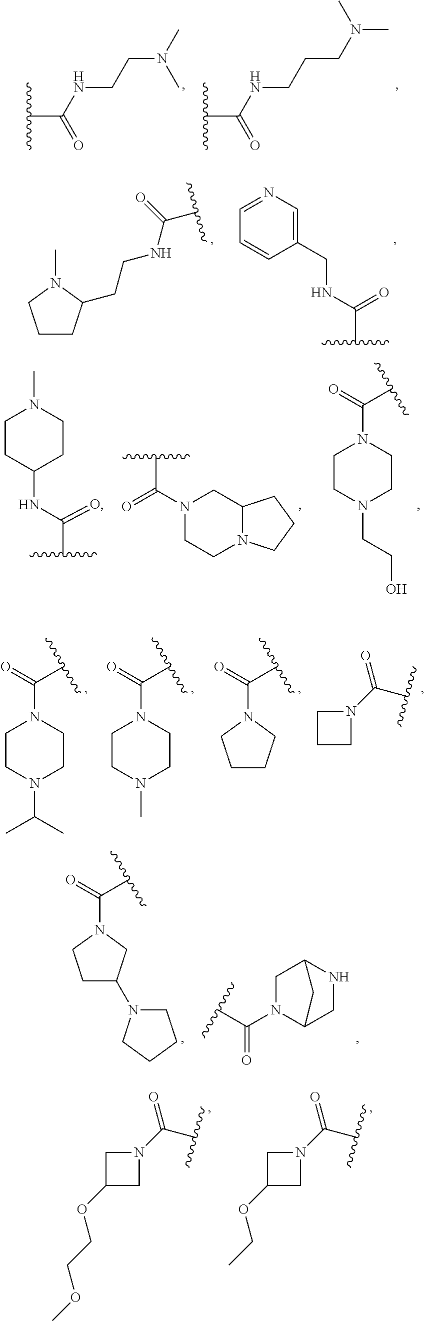

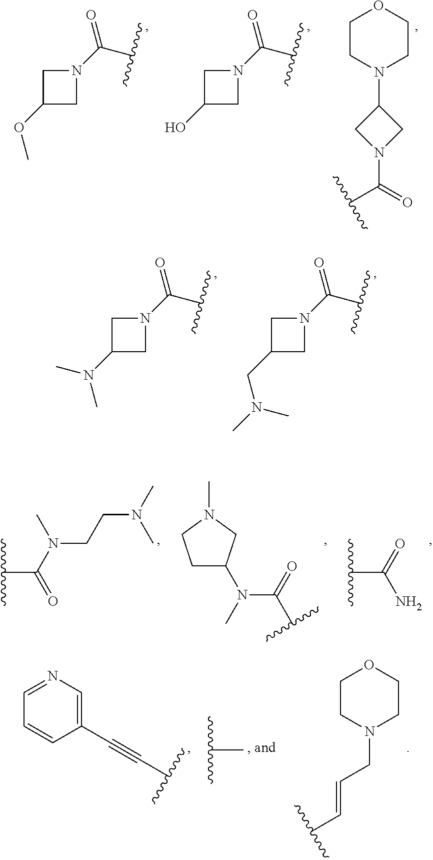

In particular embodiments of the compounds according to Formula I, two of R1, R2, and R3 are independently chosen from hydro, halo, methyl, halomethyl, and methoxy, and the remaining one of R1, R2, and R3 is chosen from:

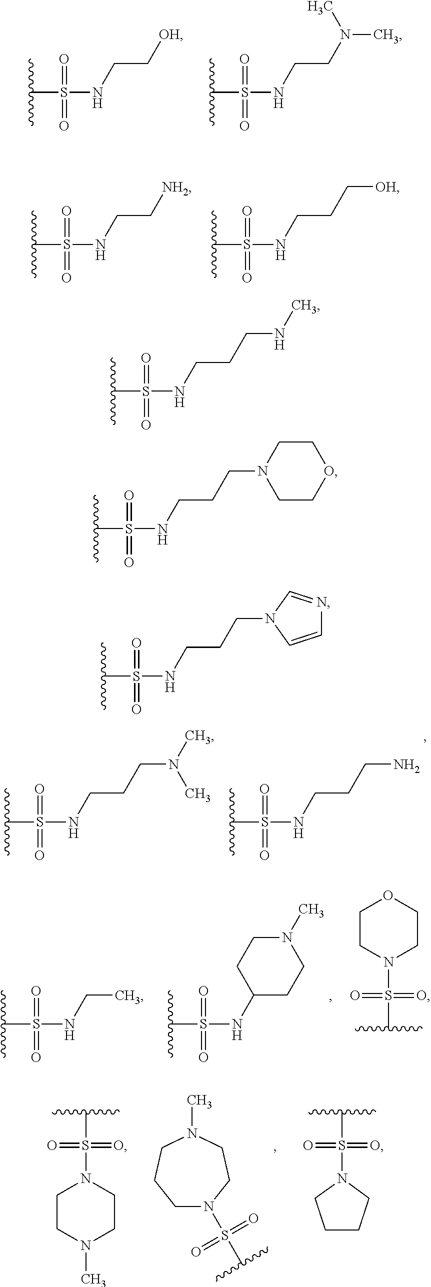

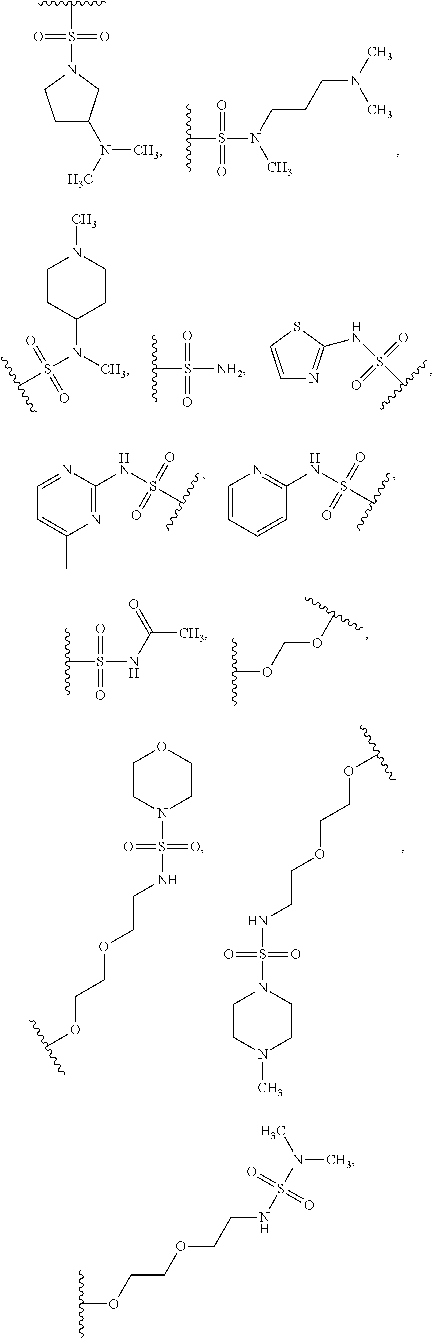

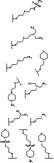

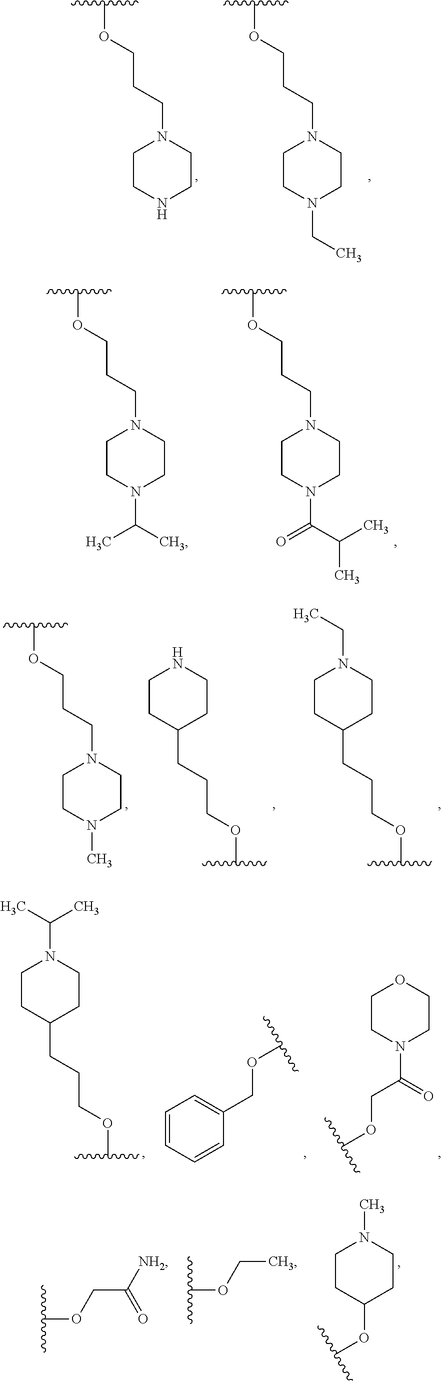

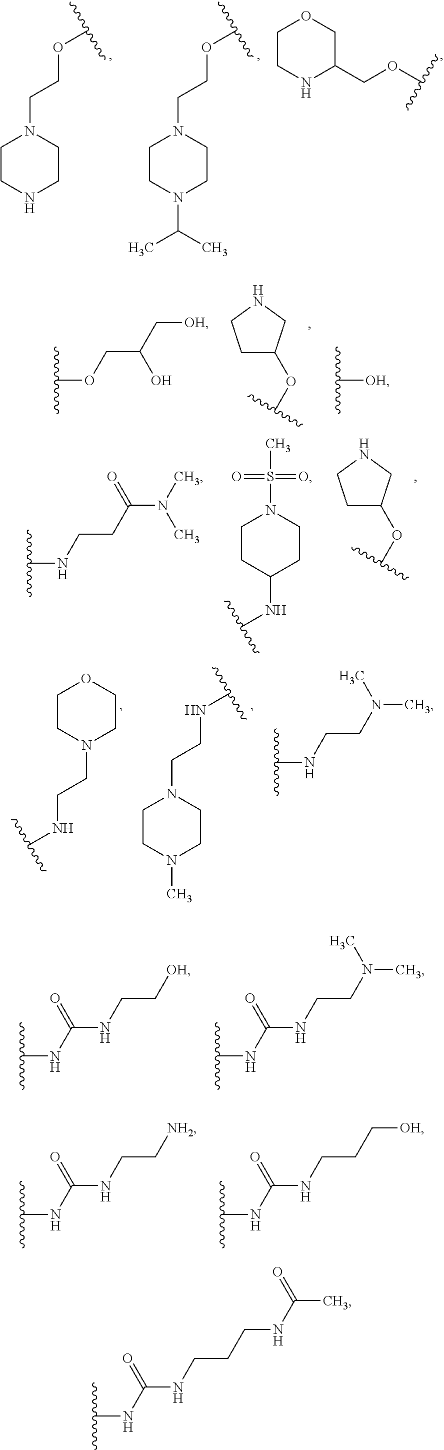

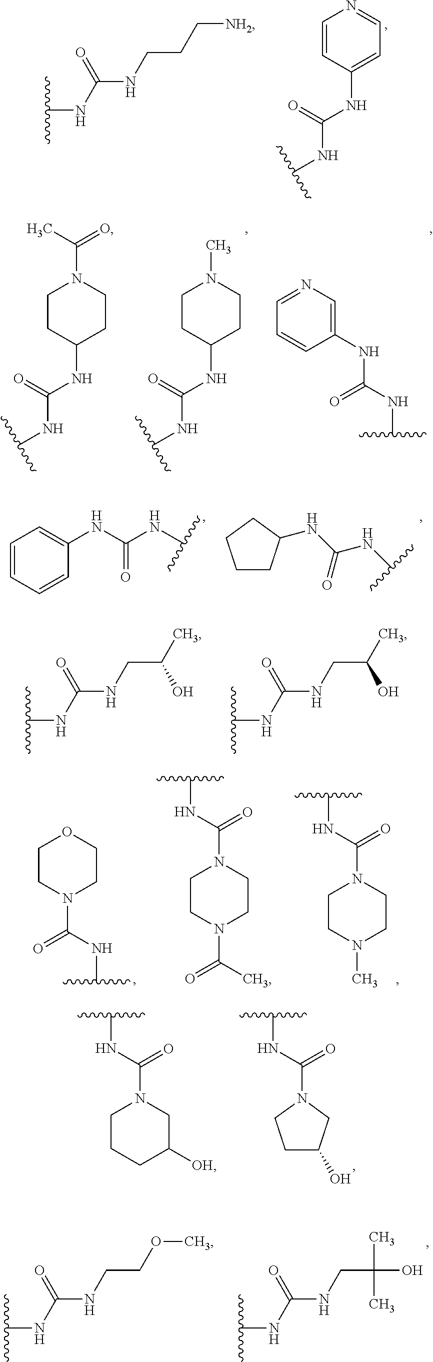

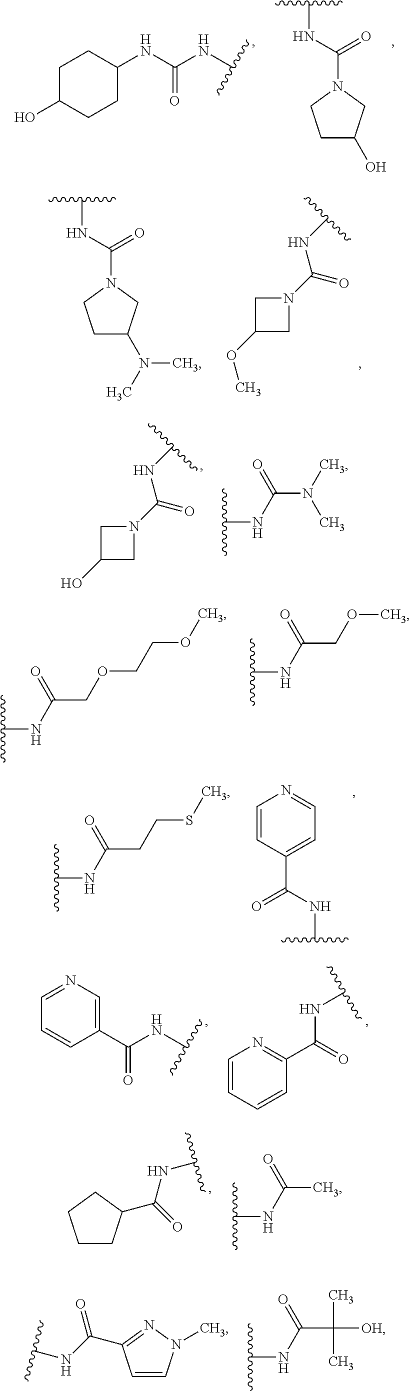

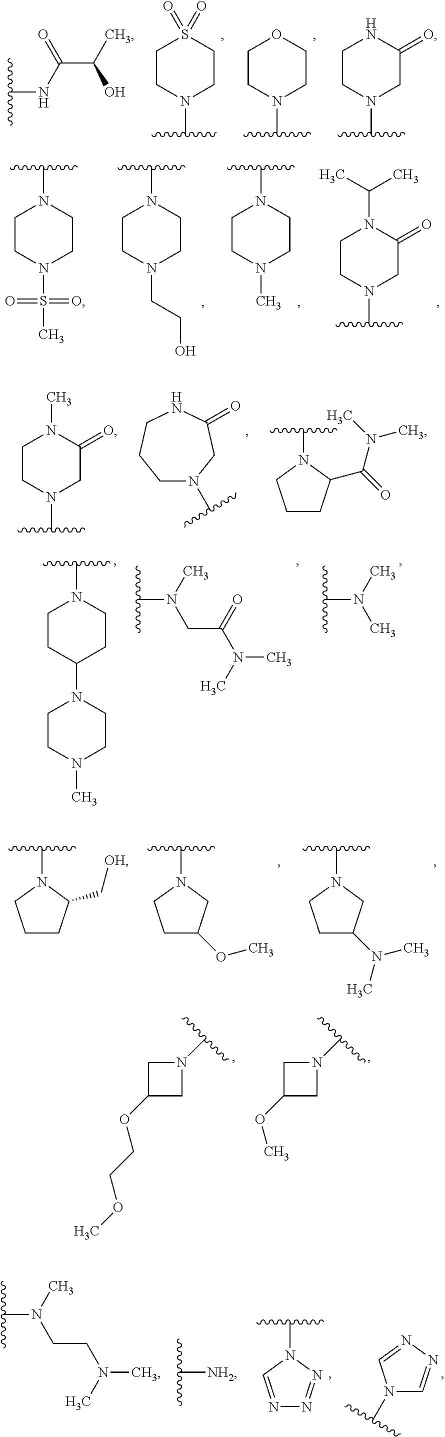

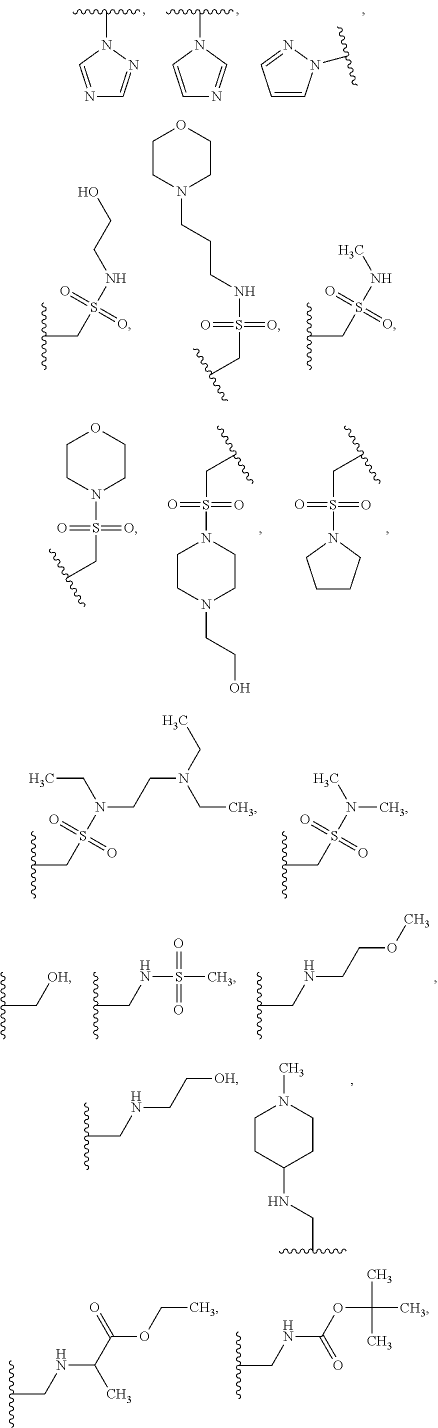

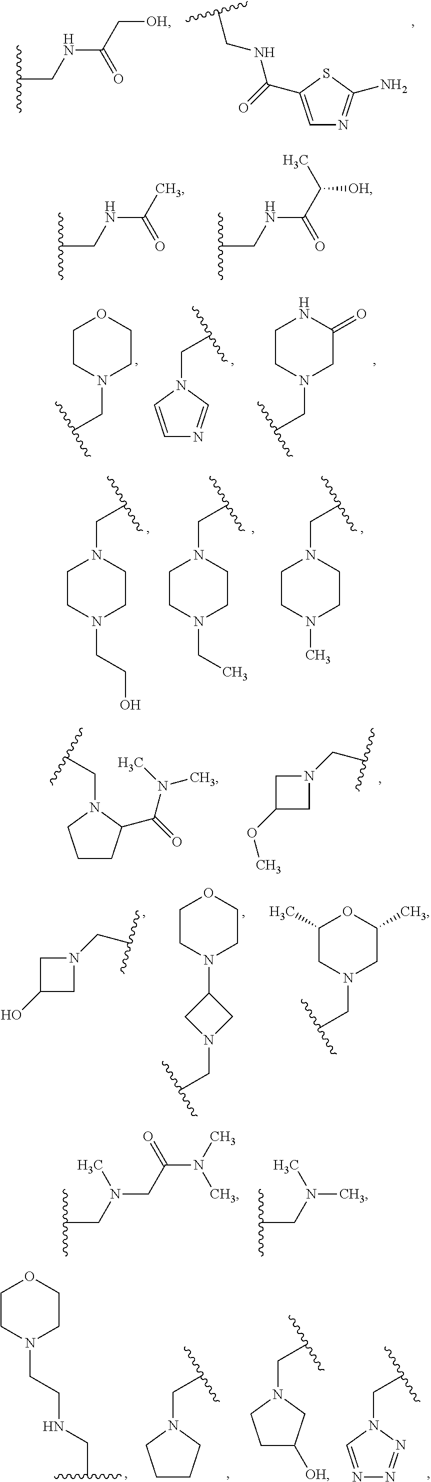

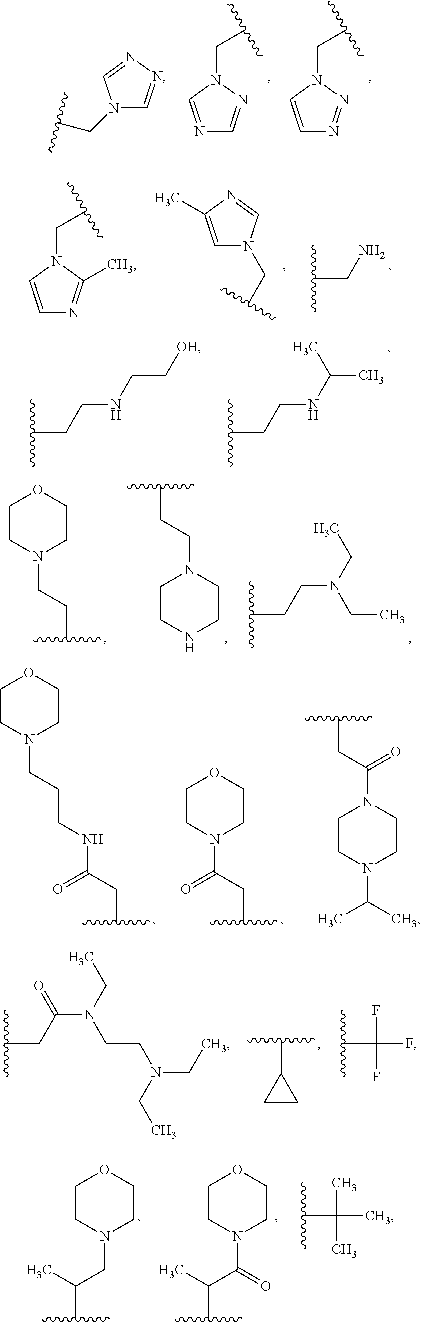

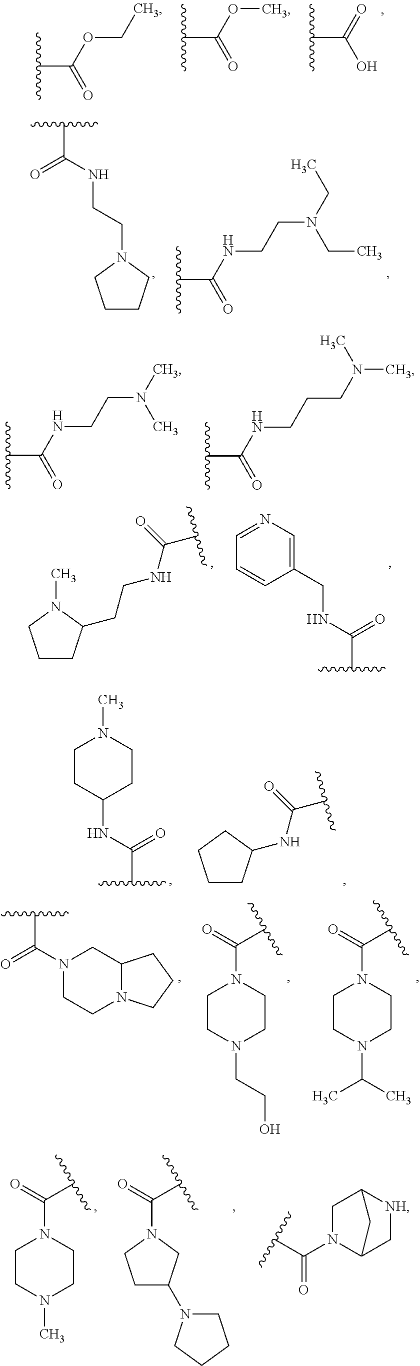

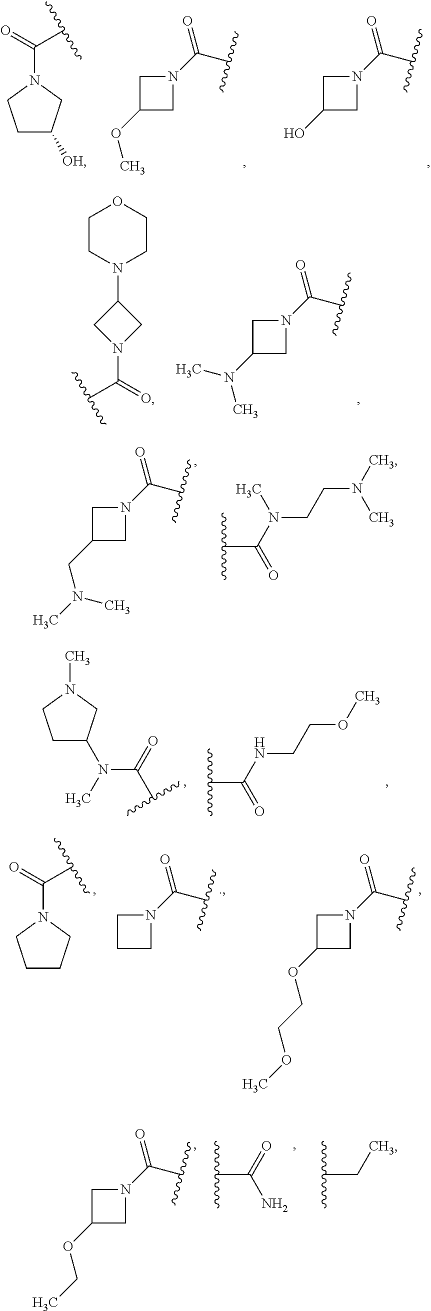

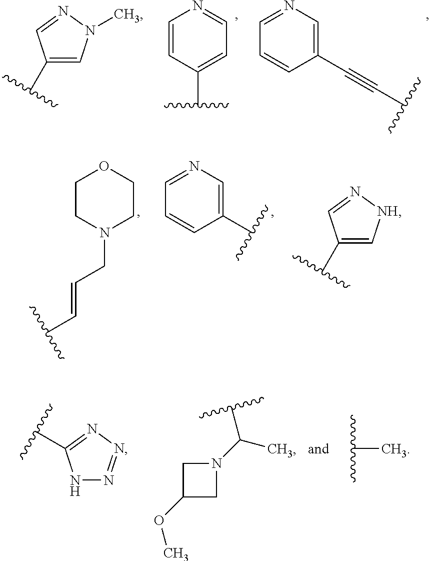

In other embodiments of the compounds according to Formula I, R1 and R2 together form a structure chosen from:

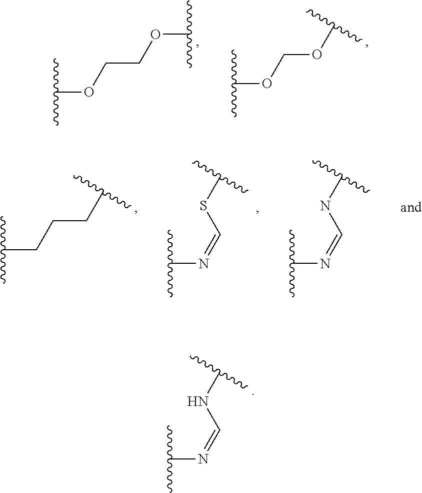 [SMILES: *OCCO* ; *OCO* ; *CCC* ; */N=C\S* ; */N=C\N* ; */N=C\N*]

In particular embodiments of the compounds according to Formula I, R4 is chosen from: —H, —Cl, —OCH₃, and

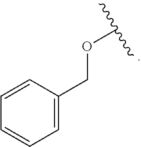 [SMILES: *OCc1ccccc1]

In particular embodiments of the compounds according to Formula I, R5 is chosen from:

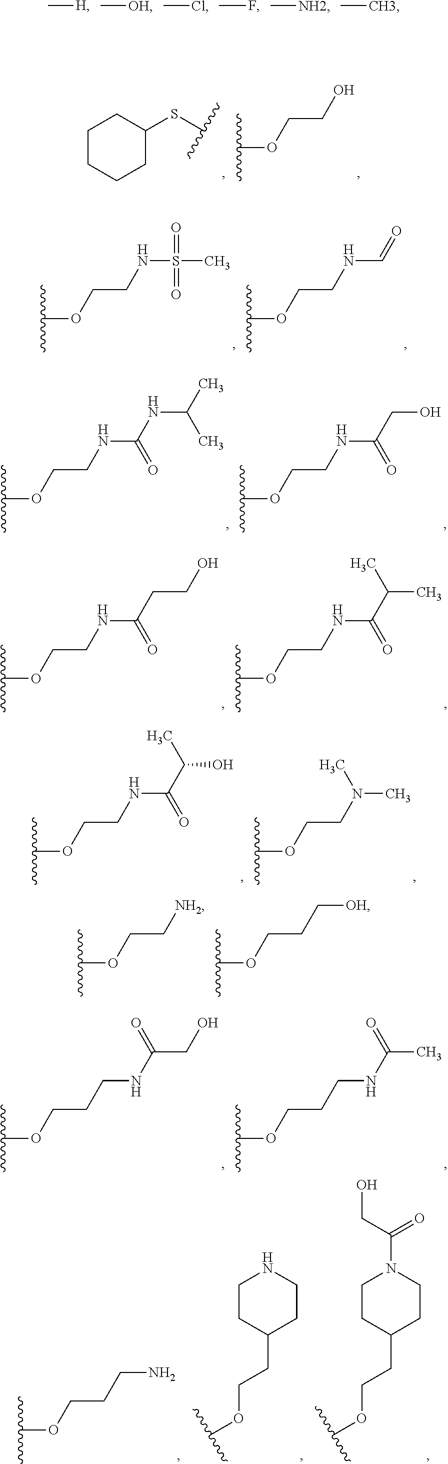

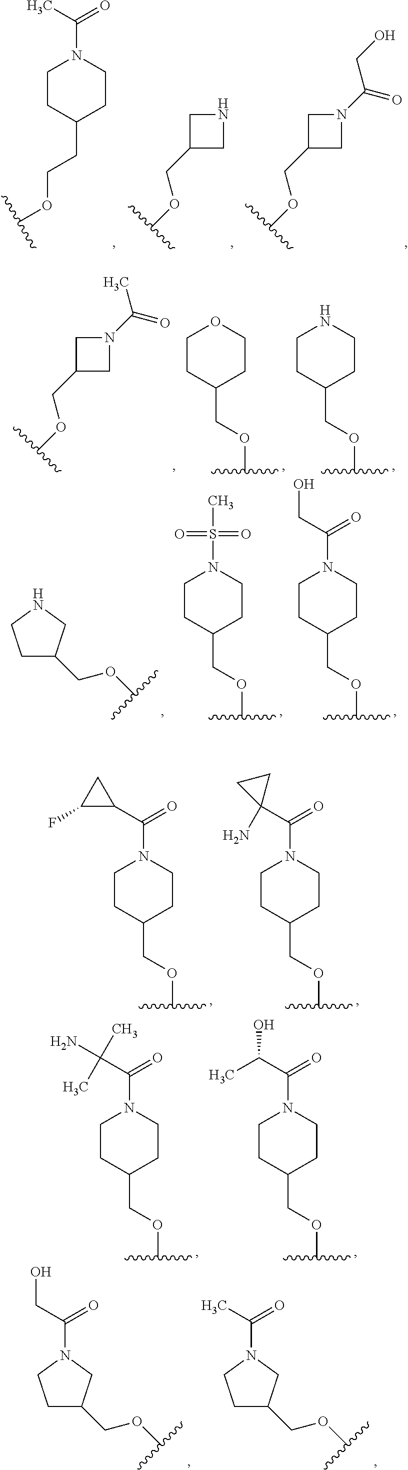

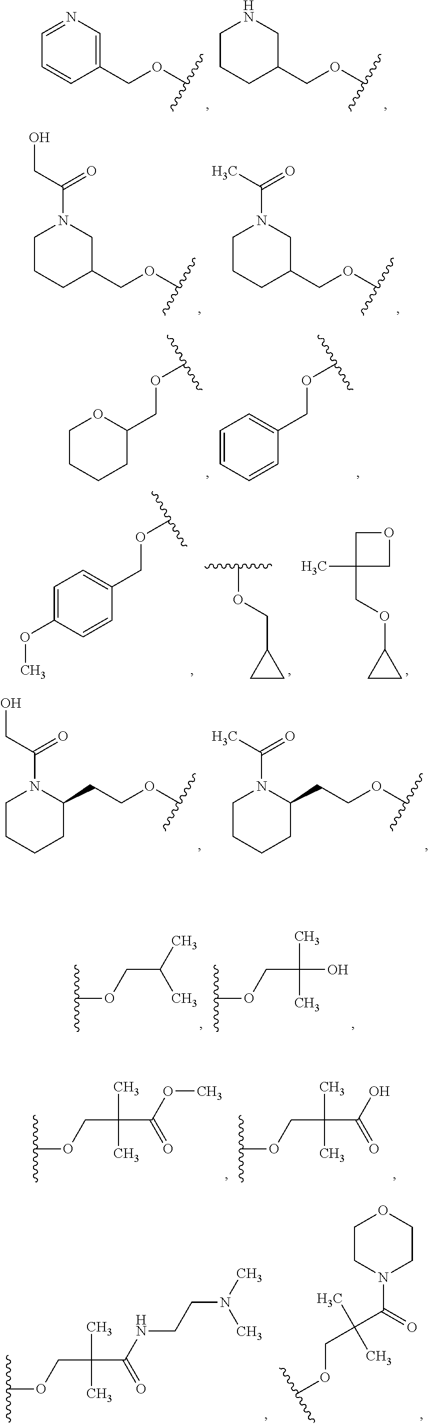

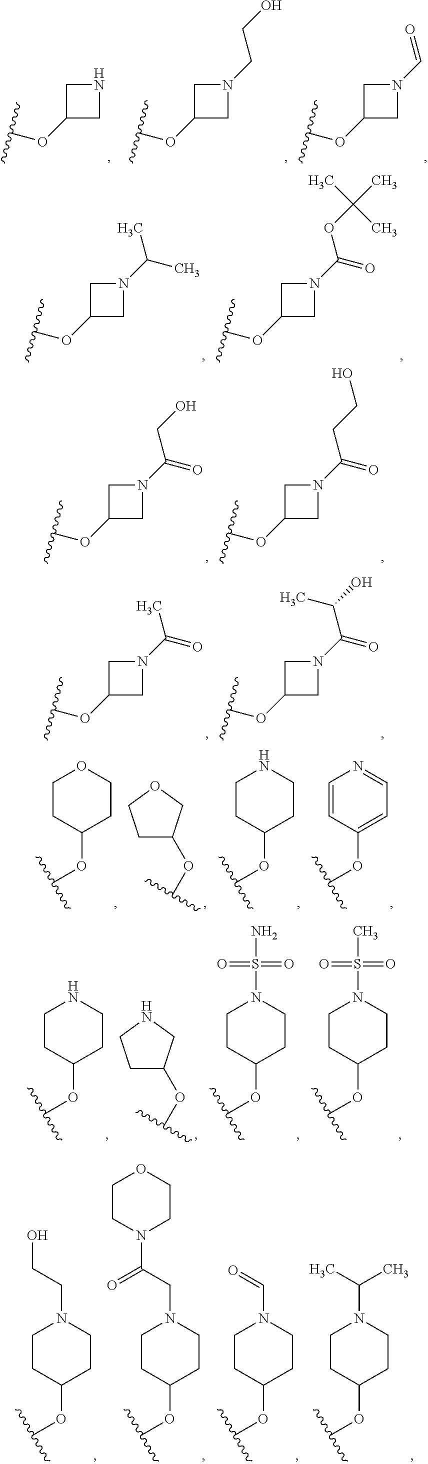

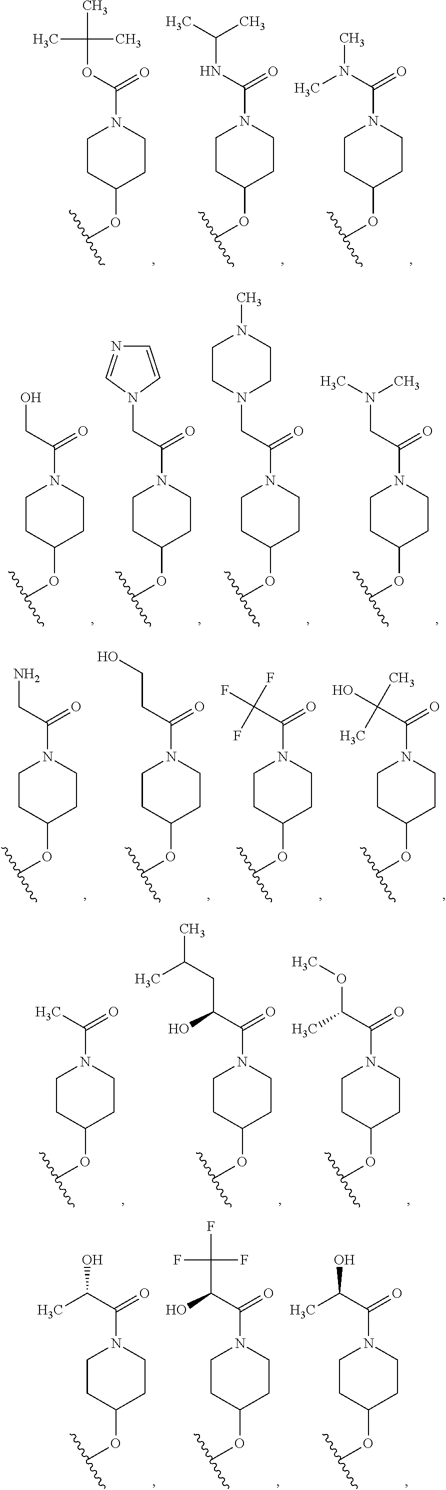

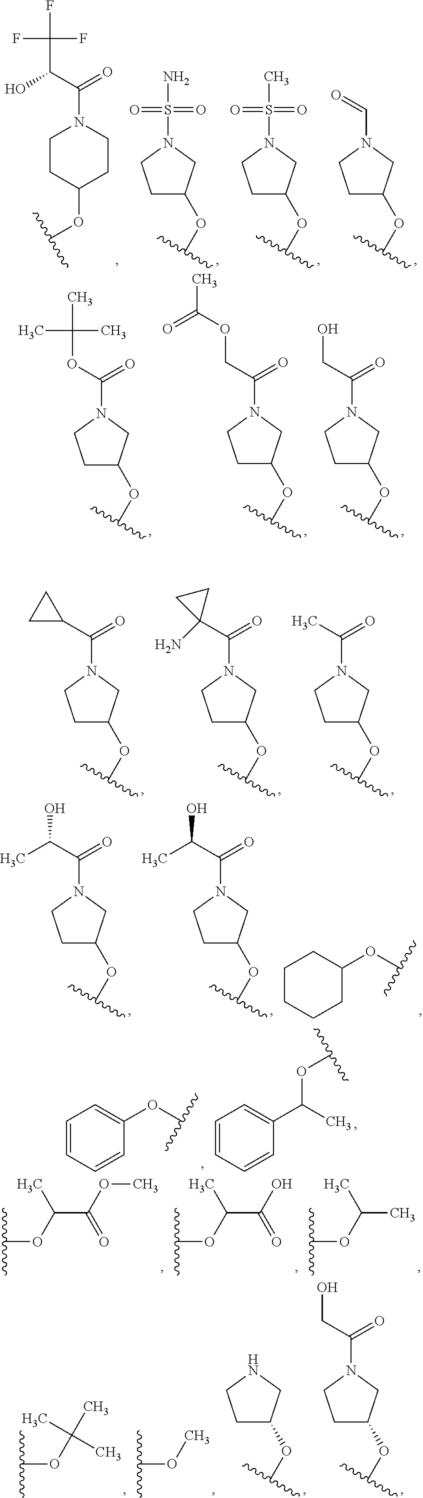

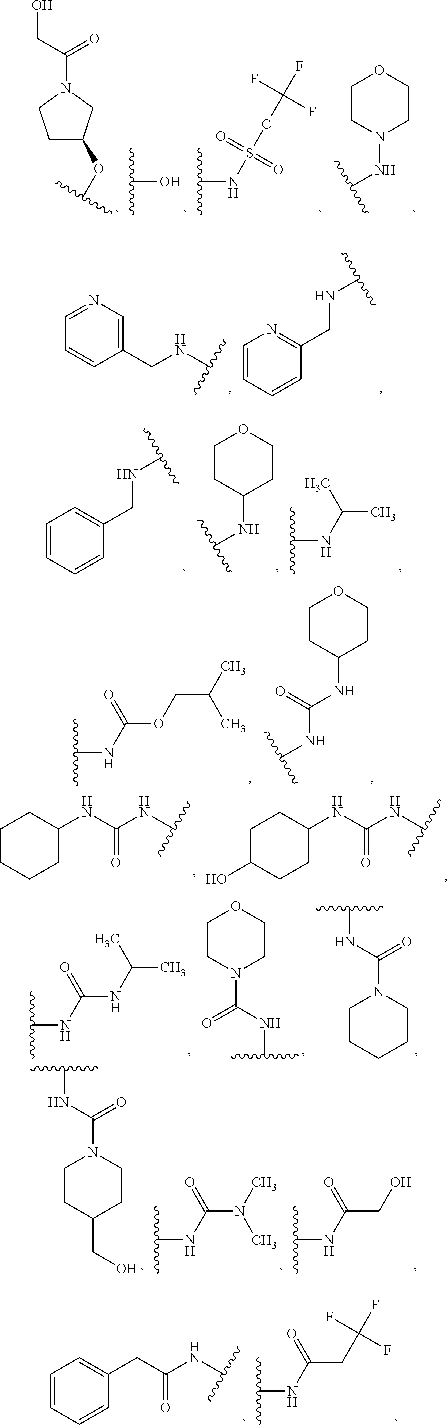

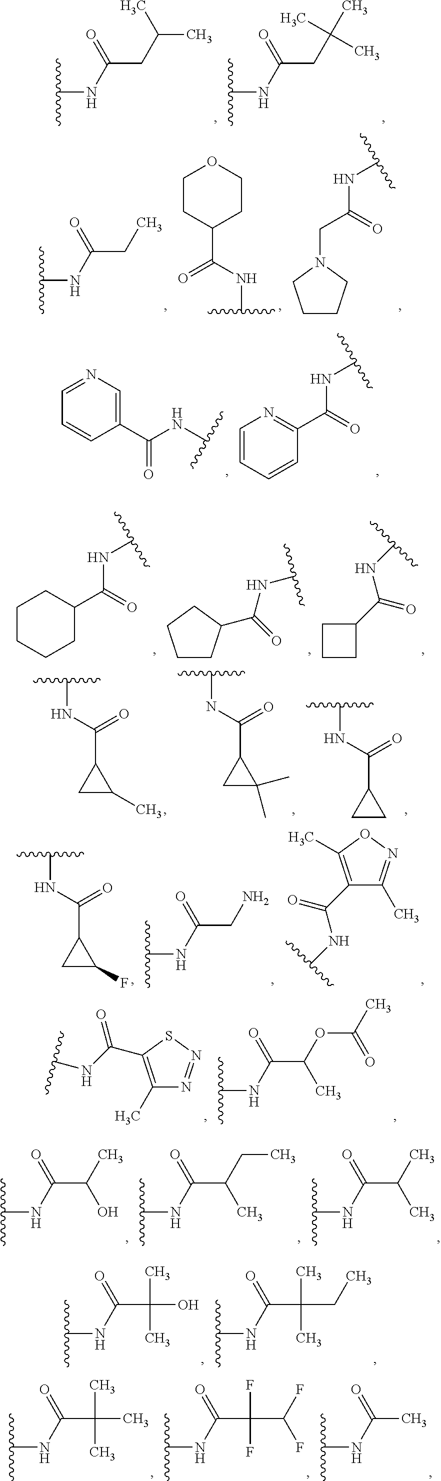

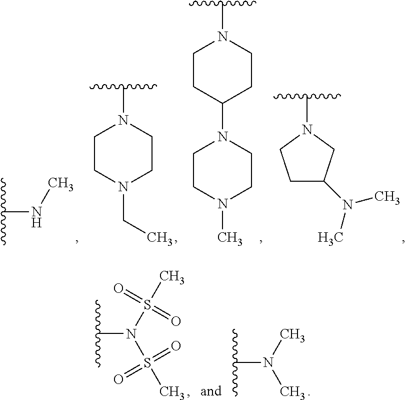

In particular embodiments, the compound according to Formula I is chosen from:
4-({4-[3-cyano-4-(tetrahydro-2H-pyran-4-yloxy)phenyl]pyrimidin-2-yl}amino)-N-[2-(dimethylamino)ethyl]-2-methoxybenzamide;
4-({4-[3-cyano-4-(tetrahydro-2H-pyran-4-yloxy)phenyl]pyrimidin-2-yl}amino)-N-[3-(dimethylamino)propyl]benzenesulfonamide;
4-({4-[3-cyano-4-({1-[(2S)-2-hydroxypropanoyl]piperidin-4-yl}oxy)phenyl]pyrimidin-2-yl}amino)-N-[3-(dimethylamino)propyl]benzamide;
5-(2-{[4-(Morpholin-4-yl)phenyl]amino}pyrimidin-4-yl)-2-(tetrahydro-2H-pyran-4-yloxy)benzonitrile;
2-({1-[(2S)-2-Hydroxypropanoyl]piperidin-4-yl}oxy)-5-(2-{[4-(morpholin-4-yl)phenyl]amino}pyrimidin-4-yl)benzonitrile;
1-[4-({4-[3-cyano-4-(tetrahydro-2H-pyran-4-yloxy)phenyl]pyrimidin-2-yl}amino)phenyl]-3-(2-hydroxyethyl)urea;
1-[4-({4-[3-cyano-4-(tetrahydro-2H-pyran-4-yloxy)phenyl]pyrimidin-2-yl}amino)phenyl]-3-pyridin-3-ylurea;
5-[2-(1,3-benzothiazol-5-ylamino)pyrimidin-4-yl]-2-(tetrahydro-2H-pyran-4-yloxy)benzonitrile;
5-[2-(1,3-benzothiazol-6-ylamino)pyrimidin-4-yl]-2-(tetrahydro-2H-pyran-4-yloxy)benzonitrile;
5-(2-{[3-methyl-4-(morpholin-4-yl)phenyl]amino}pyrimidin-4-yl)-2-(tetrahydro-2H-pyran-4-yloxy)benzonitrile;
N-[4-({4-[3-cyano-4-(tetrahydro-2H-pyran-4-yloxy)phenyl]pyrimidin-2-yl}amino)phenyl]-4-methylpiperazine-1-carboxamide;
5-[2-({4-[2-(2-aminoethoxy)ethoxy]-3-methoxyphenyl}amino)pyrimidin-4-yl]-2-(tetrahydro-2H-pyran-4-yloxy)benzonitrile;
N-(2-{2-[4-({4-[3-cyano-4-(tetrahydro-2H-pyran-4-yloxy)phenyl]pyrimidin-2-yl}amino)-2-methoxyphenoxy]ethoxy}ethyl)methanesulfonamide;
5-(2-{[3-fluoro-4-(morpholin-4-yl)phenyl]amino}pyrimidin-4-yl)-2-(tetrahydro-2H-pyran-4-yloxy)benzonitrile;
5-{2-[(3-methoxy-4-{3-[(4-methylpiperazin-1-yl)sulfonyl]propoxy}phenyl)amino]pyrimidin-4-yl}-2-(tetrahydro-2H-pyran-4-yloxy)benzonitrile;
N′-(2-{2-[4-({4-[3-cyano-4-(tetrahydro-2H-pyran-4-yloxy)phenyl]pyrimidin-2-yl}amino)-2-methoxyphenoxy]ethoxy}ethyl)-N,N-dimethylsulfuric diamide;
N-(2-{2-[4-({4-[3-cyano-4-(tetrahydro-2H-pyran-4-yloxy)phenyl]pyrimidin-2-yl}amino)-2-methoxyphenoxy]ethoxy}ethyl)-4-methylpiperazine-1-sulfonamide;
5-[2-({3-methoxy-4-[3-(morpholin-4-ylsulfonyl)propoxy]phenyl}amino)pyrimidin-4-yl]-2-(tetrahydro-2H-pyran-4-yloxy)benzonitrile;
N-(2-{2-[4-({4-[3-cyano-4-(tetrahydro-2H-pyran-4-yloxy)phenyl]pyrimidin-2-yl}amino)-2-methoxyphenoxy]ethoxy}ethyl)morpholine-4-sulfonamide;
5-(2-{[4-(2-aminoethoxy)-3-methoxyphenyl]amino}pyrimidin-4-yl)-2-(tetrahydro-2H-pyran-4-yloxy)benzonitrile;
5-[2-({3-methoxy-4-[3-(morpholin-4-yl)propoxy]phenyl}amino)pyrimidin-4-yl]-2-(tetrahydro-2H-pyran-4-yloxy)benzonitrile;
5-[2-({3-[2-(2-aminoethoxy)ethoxy]-4-methoxyphenyl}amino)pyrimidin-4-yl]-2-(tetrahydro-2H-pyran-4-yloxy)benzonitrile;
2-(Propan-2-yloxy)-5-{2-[(3,4,5-trimethoxyphenyl)amino]pyrimidin-4-yl}benzonitrile;
2-[(1-acetylpiperidin-4-yl)oxy]-5-{2-[(3,4,5-trimethoxyphenyl)amino]pyrimidin-4-yl}benzonitrile;
2-({1-[(2S)-2-hydroxypropanoyl]piperidin-4-yl}oxy)-5-[2-({4-[(4-methylpiperazin-1-yl)carbonyl]phenyl}amino)pyrimidin-4-yl]benzonitrile;
2-{[1-(hydroxyacetyl)piperidin-4-yl]oxy}-5-(2-{[3-methoxy-4-(morpholin-4-yl)phenyl]amino}pyrimidin-4-yl)benzonitrile;
N˜2˜-(4-{[4-(3-Cyano-4-methoxyphenyl)pyrimidin-2-yl]amino}-2-methoxyphenyl)-N,N,N˜2˜-trimethylglycinamide;
5-(2-{[4-(morpholin-4-yl)phenyl]amino}pyrimidin-4-yl)-2-(piperidin-4-ylmethoxy)benzonitrile;
5-(2-{[3-methoxy-4-(morpholin-4-yl)phenyl]amino}pyrimidin-4-yl)-2-(tetrahydro-2H-pyran-4-yloxy)benzonitrile;
N-[2-cyano-4-(2-{[3-methoxy-4-(morpholin-4-yl)phenyl]amino}pyrimidin-4-yl)phenyl]-2-methylpropanamide;
2-{[1-(methylsulfonyl)piperidin-4-yl]methoxy}-5-(2-{[4-(morpholin-4-yl)phenyl]amino}pyrimidin-4-yl)benzonitrile;
4-[2-cyano-4-(2-{[4-(morpholin-4-yl)phenyl]amino}pyrimidin-4-yl)phenoxy]piperidine-1-sulfonamide;
N˜2˜-[4-({4-[3-cyano-4-(tetrahydro-2H-pyran-4-yloxy)phenyl]pyrimidin-2-yl}amino)phenyl]-N,N,N˜2˜-trimethylglycinamide;
4-({4-[3-cyano-4-(tetrahydro-2H-pyran-4-yloxy)phenyl]pyrimidin-2-yl}amino)-N-[3-(1H-imidazol-1-yl)propyl]-2-methoxybenzenesulfonamide;
N-[2-Cyano-4-(2-{[3-methoxy-4-(3-oxopiperazin-1-yl)phenyl]amino}pyrimidin-4-yl)phenyl]-2-methylpropanamide;
N-[2-cyano-4-(2-{[4-(morpholin-4-yl)phenyl]amino}pyrimidin-4-yl)phenyl]cyclopropanecarboxamide;
N-[2-cyano-4-(2-{[4-(morpholin-4-yl)phenyl]amino}pyrimidin-4-yl)phenyl]-3,3,3-trifluoropropanamide;
2-{[1-(Hydroxyacetyl)pyrrolidin-3-yl]oxy}-5-(2-{[4-(morpholin-4-yl)phenyl]amino}pyrimidin-4-yl)benzonitrile;
5-(2-{[3-Chloro-4-(morpholin-4-yl)phenyl]amino}pyrimidin-4-yl)-2-methoxybenzonitrile;
5-[2-({4-[4-(methylsulfonyl)piperazin-1-yl]phenyl}amino)pyrimidin-4-yl]-2-(tetrahydro-2H-pyran-4-yloxy)benzonitrile;
4-({4-[3-cyano-4-(tetrahydro-2H-pyran-4-yloxy)phenyl]pyrimidin-2-yl}amino)-N-[3-(dimethylamino)propyl]-2-methoxybenzamide;
2-Methoxy-5-(2-{[3-methoxy-4-(3-oxo-1,4-diazepan-1-yl)phenyl]amino}pyrimidin-4-yl)benzonitrile;
5-{2-[(3,4-Dimethoxyphenyl)amino]pyrimidin-4-yl}-2-(methylamino)benzonitrile;
5-{2-[(3,4-Dimethoxyphenyl)amino]pyrimidin-4-yl}-2-(propan-2-yloxy)benzonitrile;
5-[2-({3-methoxy-4-[(4-methylpiperazin-1-yl)sulfonyl]phenyl}amino)pyrimidin-4-yl]-2-(tetrahydro-2H-pyran-4-yloxy)benzonitrile;
N˜2˜-(5-{[4-(3-Cyano-4-methoxyphenyl)pyrimidin-2-yl]amino}-2,3-dimethoxybenzyl)-N,N,N˜2˜-trimethylglycinamide;
5-{2-[(3,4-Dimethoxyphenyl)amino]pyrimidin-4-yl}-2-hydroxybenzonitrile;
2-Methoxy-5-(2-{[3-methoxy-4-(4-methyl-3-oxopiperazin-1-yl)phenyl]amino}pyrimidin-4-yl)benzonitrile;
5-(2-{[3-(Hydroxymethyl)-4,5-dimethoxyphenyl]amino}pyrimidin-4-yl)-2-methoxybenzonitrile;
N-[2-cyano-4-(2-{[4-(morpholin-4-yl)phenyl]amino}pyrimidin-4-yl)phenyl]-4-methyl-1,2,3-thiadiazole-5-carboxamide;
2-Hydroxy-5-(2-{[4-(morpholin-4-yl)phenyl]amino}pyrimidin-4-yl)benzonitrile;
2-[5-({4-[3-cyano-4-(tetrahydro-2H-pyran-4-yloxy)phenyl]pyrimidin-2-yl}amino)-2-methoxyphenoxy]acetamide;
2-[(1-Acetylpiperidin-4-yl)oxy]-5-(2-{[3-methoxy-4-(3-oxopiperazin-1-yl)phenyl]amino}pyrimidin-4-yl)benzonitrile;
4-({4-[3-cyano-4-(tetrahydro-2H-pyran-4-yloxy)phenyl]pyrimidin-2-yl}amino)-N-(3-hydroxypropyl)-2-methoxybenzenesulfonamide;
2-Methoxy-5-(2-{[3-methoxy-4-(morpholin-4-yl)phenyl]amino}pyrimidin-4-yl)benzonitrile;
5-(2-{[4-(morpholin-4-yl)phenyl]amino}pyrimidin-4-yl)-2-(tetrahydro-2H-pyran-4-ylmethoxy)benzonitrile;
2-tert-Butoxy-5-(2-{[4-(morpholin-4-yl)phenyl]amino}pyrimidin-4-yl)benzonitrile;
2-(Cyclohexyloxy)-5-(2-{[4-(morpholin-4-yl)phenyl]amino}pyrimidin-4-yl)benzonitrile;
5-{2-[(4-{[1-(methylsulfonyl)piperidin-4-yl]amino}phenyl)amino]pyrimidin-4-yl}-2-(tetrahydro-2H-pyran-4-yloxy)benzonitrile;
4-({4-[3-cyano-4-(tetrahydro-2H-pyran-4-yloxy)phenyl]pyrimidin-2-yl}amino)-2-methoxy-N-[3-(morpholin-4-yl)propyl]benzenesulfonamide;
5-(2-{[4-(4-methylpiperazin-1-yl)phenyl]amino}pyrimidin-4-yl)-2-(tetrahydro-2H-pyran-4-yloxy)benzonitrile;
N-{3-[2-cyano-4-(2-{[4-(morpholin-4-yl)phenyl]amino}pyrimidin-4-yl)phenoxy]propyl}-2-hydroxyacetamide;
5-{2-[(4-Aminophenyl)amino]pyrimidin-4-yl}-2-(tetrahydro-2H-pyran-4-yloxy)benzonitrile;
2-{[1-(Hydroxyacetyl)piperidin-4-yl]oxy}-5-(2-{[4-(morpholin-4-yl)phenyl]amino}pyrimidin-4-yl)benzonitrile;
5-(2-{[4-(Morpholin-4-yl)phenyl]amino}pyrimidin-4-yl)-2-(propan-2-yloxy)benzonitrile;
5-{2-[(3,4-Dimethoxyphenyl)amino]pyrimidin-4-yl}-2-(dimethylamino)benzonitrile;
2-({1-[(2S)-2-hydroxypropanoyl]piperidin-4-yl}oxy)-5-(2-{[3-methoxy-4-(morpholin-4-yl)phenyl]amino}pyrimidin-4-yl)benzonitrile;
2-(3-Hydroxypropoxy)-5-(2-{[4-(morpholin-4-yl)phenyl]amino}pyrimidin-4-yl)benzonitrile;
5-(2-{[4-(Morpholin-4-yl)phenyl]amino}pyrimidin-4-yl)-2-(propan-2-ylamino)benzonitrile;
4-({4-[3-cyano-4-(tetrahydro-2H-pyran-4-yloxy)phenyl]pyrimidin-2-yl}amino)-2-methoxy-N-methyl-N-(1-methylpiperidin-4-yl)benzenesulfonamide;
(2S)—N-[2-cyano-4-(2-{[4-(morpholin-4-yl)phenyl]amino}pyrimidin-4-yl)phenyl]-2-fluorocyclopropanecarboxamide;
2-{[1-(hydroxyacetyl)pyrrolidin-3-yl]oxy}-5-(2-{[3-methoxy-4-(morpholin-4-yl)phenyl]amino}pyrimidin-4-yl)benzonitrile;
3-[2-cyano-4-(2-{[4-(morpholin-4-yl)phenyl]amino}pyrimidin-4-yl)phenoxy]pyrrolidine-1-sulfonamide;
2-(2-Hydroxy-2-methylpropoxy)-5-(2-{[4-(morpholin-4-yl)phenyl]amino}pyrimidin-4-yl)benzonitrile;
methyl 4-({4-[3-cyano-4-(tetrahydro-2H-pyran-4-yloxy)phenyl]pyrimidin-2-yl}amino)-2-methoxybenzoate;
4-({4-[3-cyano-4-(tetrahydro-2H-pyran-4-yloxy)phenyl]pyrimidin-2-yl}amino)-N-[3-(dimethylamino)propyl]-2-methoxybenzenesulfonamide;
2-(2-Hydroxyethoxy)-5-(2-{[4-(morpholin-4-yl)phenyl]amino}pyrimidin-4-yl)benzonitrile;
2-[(1-formylpiperidin-4-yl)oxy]-5-(2-{[4-(morpholin-4-yl)phenyl]amino}pyrimidin-4-yl)benzonitrile;
2-{[1-(Methylsulfonyl)piperidin-4-yl]oxy}-5-(2-{[4-(morpholin-4-yl)phenyl]amino}pyrimidin-4-yl)benzonitrile;
4-({4-[3-cyano-4-(tetrahydro-2H-pyran-4-yloxy)phenyl]pyrimidin-2-yl}amino)-2-methoxy-N-(1-methylpiperidin-4-yl)benzenesulfonamide;
5-[2-({3-methoxy-4-[3-(4-methylpiperazin-1-yl)propoxy]phenyl}amino)pyrimidin-4-yl]-2-(tetrahydro-2H-pyran-4-yloxy)benzonitrile;
5-(2-{[4-(Morpholin-4-yl)phenyl]amino}pyrimidin-4-yl)-2-(tetrahydrofuran-3-yloxy)benzonitrile;
5-{2-[(4-hydroxy-3-methoxyphenyl)amino]pyrimidin-4-yl}-2-(tetrahydro-2H-pyran-4-yloxy)benzonitrile;
2-(2-Methylpropoxy)-5-(2-{[4-(morpholin-4-yl)phenyl]amino}pyrimidin-4-yl)benzonitrile;
5-{2-[(3-{[(1-Methylpiperidin-4-yl)amino]methyl}phenyl)amino]pyrimidin-4-yl}-2-(tetrahydro-2H-pyran-4-yloxy)benzonitrile;
4-({4-[3-cyano-4-(tetrahydro-2H-pyran-4-yloxy)phenyl]pyrimidin-2-yl}amino)-2-methoxy-N-(pyridin-3-ylmethyl)benzamide;
4-({4-[3-cyano-4-({1-[(2S)-2-hydroxypropanoyl]piperidin-4-yl}oxy)phenyl]pyrimidin-2-yl}amino)-N-[2-(dimethylamino)ethyl]-2-methoxybenzamide;
2-(Tetrahydro-2H-pyran-4-yloxy)-5-{2-[(3,4,5-trimethoxyphenyl)amino]pyrimidin-4-yl}benzonitrile;
4-({4-[3-cyano-4-(tetrahydro-2H-pyran-4-yloxy)phenyl]pyrimidin-2-yl}amino)-2-methoxy-N-[2-(1-methylpyrrolidin-2-yl)ethyl]benzamide;
4-({4-[3-cyano-4-(tetrahydro-2H-pyran-4-yloxy)phenyl]pyrimidin-2-yl}amino)-2-methoxybenzamide;
2-Hydroxy-5-(2-{[3-methoxy-4-(3-oxopiperazin-1-yl)phenyl]amino}pyrimidin-4-yl)benzonitrile;
5-(2-{[3-cyclopropyl-4-(morpholin-4-yl)phenyl]amino}pyrimidin-4-yl)-2-(tetrahydro-2H-pyran-4-yloxy)benzonitrile;
4-({4-[3-cyano-4-({1-[(2S)-2-hydroxypropanoyl]piperidin-4-yl}oxy)phenyl]pyrimidin-2-yl}amino)-N-[2-(dimethylamino)ethyl]-N-methylbenzamide;
4-({4-[3-cyano-4-(tetrahydro-2H-pyran-4-yloxy)phenyl]pyrimidin-2-yl}amino)-N-[2-(dimethylamino)ethyl]benzenesulfonamide;
5-(2-{[4-(4-Methylpiperazin-1-yl)phenyl]amino}pyrimidin-4-yl)-2-(propan-2-yloxy)benzonitrile;
2-Methoxy-5-{2-[(3,4,5-trimethoxyphenyl)amino]pyrimidin-4-yl}benzonitrile;
5-[2-({3-methoxy-4-[(4-methylpiperazin-1-yl)carbonyl]phenyl}amino)pyrimidin-4-yl]-2-(tetrahydro-2H-pyran-4-yloxy)benzonitrile;
4-({4-[3-cyano-4-({1-[(2S)-2-hydroxypropanoyl]piperidin-4-yl}oxy)phenyl]pyrimidin-2-yl}amino)-N-[2-(dimethylamino)ethyl]benzamide;
2-Methoxy-5-(2-{[3-methoxy-4-(3-oxopiperazin-1-yl)phenyl]amino}pyrimidin-4-yl)benzonitrile;
4-({4-[3-cyano-4-(tetrahydro-2H-pyran-4-yloxy)phenyl]pyrimidin-2-yl}amino)-N-(1-methylpiperidin-4-yl)benzenesulfonamide;
3-{[4-(3-Cyanophenyl)pyrimidin-2-yl]amino}benzenesulfonamide;
5-(2-{[3-chloro-4-(morpholin-4-yl)phenyl]amino}pyrimidin-4-yl)-2-(tetrahydro-2H-pyran-4-yloxy)benzonitrile;
4-({4-[3-cyano-4-({1-[(2S)-2-hydroxypropanoyl]piperidin-4-yl}oxy)phenyl]pyrimidin-2-yl}amino)-N-[3-(dimethylamino)propyl]-2-methoxybenzamide;
5-{2-[(4-{[3-(dimethylamino)azetidin-1-yl]carbonyl}-3-methoxyphenyl)amino]pyrimidin-4-yl}-2-(tetrahydro-2H-pyran-4-yloxy)benzonitrile;
4-({4-[3-cyano-4-(tetrahydro-2H-pyran-4-yloxy)phenyl]pyrimidin-2-yl}amino)-2-methoxy-N-(1-methylpiperidin-4-yl)benzamide;
4-({4-[3-cyano-4-(tetrahydro-2H-pyran-4-yloxy)phenyl]pyrimidin-2-yl}amino)-2-methoxy-N-methyl-N-(1-methylpyrrolidin-3-yl)benzamide;
5-[2-({3-Methoxy-4-[(4-methyl-1,4-diazepan-1-yl)sulfonyl]phenyl}amino)pyrimidin-4-yl]-2-(tetrahydro-2H-pyran-4-yloxy)benzonitrile;
5-{2-[(3-Aminophenyl)amino]pyrimidin-4-yl}-2-(tetrahydro-2H-pyran-4-yloxy)benzonitrile;
5-(2-{[3-methoxy-4-(pyrrolidin-1-ylsulfonyl)phenyl]amino}pyrimidin-4-yl)-2-(tetrahydro-2H-pyran-4-yloxy)benzonitrile;
5-(2-{[3-(hydroxymethyl)phenyl]amino}pyrimidin-4-yl)-2-(tetrahydro-2H-pyran-4-yloxy)benzonitrile;
4-({4-[3-cyano-4-(tetrahydro-2H-pyran-4-yloxy)phenyl]pyrimidin-2-yl}amino)-2-methoxy-N-[3-(methylamino)propyl]benzenesulfonamide;
4-({4-[3-cyano-4-(tetrahydro-2H-pyran-4-yloxy)phenyl]pyrimidin-2-yl}amino)-N-[3-(dimethylamino)propyl]-2-methoxy-N-methylbenzenesulfonamide;
5-{2-[(4-{[3-(dimethylamino)pyrrolidin-1-yl]sulfonyl}-3-methoxyphenyl)amino]pyrimidin-4-yl}-2-(tetrahydro-2H-pyran-4-yloxy)benzonitrile;
1-[4-({4-[3-Cyano-4-(tetrahydro-2H-pyran-4-yloxy)phenyl]pyrimidin-2-yl}amino)phenyl]-N,N-dimethylmethanesulfonamide;
1-[4-({4-[3-Cyano-4-(tetrahydro-2H-pyran-4-yloxy)phenyl]pyrimidin-2-yl}amino)phenyl]-N-(2-hydroxyethyl)methanesulfonamide;
5-[2-({4-[(Pyrrolidin-1-ylsulfonyl)methyl]phenyl}amino)pyrimidin-4-yl]-2-(tetrahydro-2H-pyran-4-yloxy)benzonitrile;
5-[2-({4-[(Morpholin-4-ylsulfonyl)methyl]phenyl}amino)pyrimidin-4-yl]-2-(tetrahydro-2H-pyran-4-yloxy)benzonitrile;
1-[4-({4-[3-Cyano-4-(tetrahydro-2H-pyran-4-yloxy)phenyl]pyrimidin-2-yl}amino)phenyl]-N-[3-(morpholin-4-yl)propyl]methanesulfonamide
5-(2-{[4-({[4-(2-Hydroxyethyl)piperazin-1-yl]sulfonyl}methyl)phenyl]amino}pyrimidin-4-yl)-2-(tetrahydro-2H-pyran-4-yloxy)benzonitrile;
1-[4-({4-[3-cyano-4-(tetrahydro-2H-pyran-4-yloxy)phenyl]pyrimidin-2-yl}amino)phenyl]-N-methylmethanesulfonamide;
N-[2-cyano-4-(2-{[4-(morpholin-4-yl)phenyl]amino}pyrimidin-4-yl)phenyl]-2-methylcyclopropanecarboxamide;
2-({1-[(2R)-2-Hydroxypropanoyl]piperidin-4-yl}oxy)-3-methoxy-5-(2-{[4-(morpholin-4-yl)phenyl]amino}pyrimidin-4-yl)benzonitrile;
5-[2-({4-[4-(2-Hydroxyethyl)piperazin-1-yl]phenyl}amino)pyrimidin-4-yl]-2-[(3-methyloxetan-3-yl)methoxy]benzonitrile;
2-(Cyclopropylmethoxy)-5-(2-{[4-(morpholin-4-yl)phenyl]amino}pyrimidin-4-yl)benzonitrile;
2-(Cyclopropylmethoxy)-5-[2-({4-[4-(2-hydroxyethyl)piperazin-1-yl]phenyl}amino)pyrimidin-4-yl]benzonitrile;
3-Methoxy-5-(2-{[4-(morpholin-4-yl)phenyl]amino}pyrimidin-4-yl)-2-(piperidin-4-yloxy)benzonitrile;
5-[2-({4-[4-(2-Hydroxyethyl)piperazin-1-yl]phenyl}amino)pyrimidin-4-yl]-2-(2-methylpropoxy)benzonitrile;
2-[(3-Methyloxetan-3-yl)methoxy]-5-(2-{[4-(morpholin-4-yl)phenyl]amino}pyrimidin-4-yl)benzonitrile;
5-[2-({4-[4-(2-Hydroxyethyl)piperazin-1-yl]phenyl}amino)pyrimidin-4-yl]-3-methoxy-2-(tetrahydro-2H-pyran-4-yloxy)benzonitrile;
3-methoxy-5-(2-{[4-(morpholin-4-yl)phenyl]amino}pyrimidin-4-yl)-2-(tetrahydro-2H-pyran-4-yloxy)benzonitrile;
2-{[(3R)-1-(hydroxyacetyl)pyrrolidin-3-yl]oxy}-5-(2-{[4-(morpholin-4-yl)phenyl]amino}pyrimidin-4-yl)benzonitrile;
5-{2-[(3-Methoxy-4-{[3-(morpholin-4-yl)azetidin-1-yl]carbonyl}phenyl)amino]pyrimidin-4-yl}-2-(tetrahydro-2H-pyran-4-yloxy)benzonitrile;
5-{2-[(4-{[4-(2-Hydroxyethyl)piperazin-1-yl]carbonyl}-3-methoxyphenyl)amino]pyrimidin-4-yl}-2-(tetrahydro-2H-pyran-4-yloxy)benzonitrile;
5-{2-[(4-{[4-(2-Hydroxyethyl)piperazin-1-yl]methyl}-3-methoxyphenyl)amino]pyrimidin-4-yl}-2-(tetrahydro-2H-pyran-4-yloxy)benzonitrile;
5-{2-[(3-Methoxy-4-{[(2-methoxyethyl)amino]methyl}phenyl)amino]pyrimidin-4-yl}-2-(tetrahydro-2H-pyran-4-yloxy)benzonitrile;
5-[2-({3-Methoxy-4-[(4-methylpiperazin-1-yl)methyl]phenyl}amino)pyrimidin-4-yl]-2-(tetrahydro-2H-pyran-4-yloxy)benzonitrile;
5-{2-[(4-{[(2R,6S)-2,6-Dimethylmorpholin-4-yl]methyl}-3-methoxyphenyl)amino]pyrimidin-4-yl}-2-(tetrahydro-2H-pyran-4-yloxy)benzonitrile;
5-{2-[(3-Methoxy-4-{[3-(morpholin-4-yl)azetidin-1-yl]methyl}phenyl)amino]pyrimidin-4-yl}-2-(tetrahydro-2H-pyran-4-yloxy)benzonitrile;
5-[2-({3-Methoxy-4-[(3-methoxyazetidin-1-yl)methyl]phenyl}amino)pyrimidin-4-yl]-2-(tetrahydro-2H-pyran-4-yloxy)benzonitrile;
5-[2-({3-Methoxy-4-[(3-methoxyazetidin-1-yl)carbonyl]phenyl}amino)pyrimidin-4-yl]-2-(tetrahydro-2H-pyran-4-yloxy)benzonitrile;
5-[2-({4-[(3-Hydroxyazetidin-1-yl)carbonyl]-3-methoxyphenyl}amino)pyrimidin-4-yl]-2-(tetrahydro-2H-pyran-4-yloxy)benzonitrile;
5-(2-{[4-(aminomethyl)phenyl]amino}pyrimidin-4-yl)-2-(tetrahydro-2H-pyran-4-yloxy)benzonitrile;
5-[2-({4-[(3-methoxyazetidin-1-yl)methyl]phenyl}amino)pyrimidin-4-yl]-2-(tetrahydro-2H-pyran-4-yloxy)benzonitrile;
5-{2-[(4-{[(2-methoxyethyl)amino]methyl}phenyl)amino]pyrimidin-4-yl}-2-(tetrahydro-2H-pyran-4-yloxy)benzonitrile;
ethyl N-[4-({4-[3-cyano-4-(tetrahydro-2H-pyran-4-yloxy)phenyl]pyrimidin-2-yl}amino)benzyl]alaninate;
2-amino-N-[4-({4-[3-cyano-4-(tetrahydro-2H-pyran-4-yloxy)phenyl]pyrimidin-2-yl}amino)benzyl]-1,3-thiazole-5-carboxamide;
N-[4-({4-[3-cyano-4-(tetrahydro-2H-pyran-4-yloxy)phenyl]pyrimidin-2-yl}amino)benzyl]acetamide;
N-[4-({4-[3-cyano-4-(tetrahydro-2H-pyran-4-yloxy)phenyl]pyrimidin-2-yl}amino)benzyl]methanesulfonamide;
(2S)—N-[4-({4-[3-cyano-4-(tetrahydro-2H-pyran-4-yloxy)phenyl]pyrimidin-2-yl}amino)benzyl]-2-hydroxypropanamide;
N-[4-({4-[3-cyano-4-(tetrahydro-2H-pyran-4-yloxy)phenyl]pyrimidin-2-yl}amino)benzyl]-2-hydroxyacetamide;
5-(2-{[4-(2,5-diazabicyclo[2.2.1]hept-2-ylcarbonyl)-3-methoxyphenyl]amino}pyrimidin-4-yl)-2-(tetrahydro-2H-pyran-4-yloxy)benzonitrile;
5-[2-({4-[(3-hydroxyazetidin-1-yl)methyl]phenyl}amino)pyrimidin-4-yl]-2-(tetrahydro-2H-pyran-4-yloxy)benzonitrile;
5-(2-{[4-(hydroxymethyl)-3-methoxyphenyl]amino}pyrimidin-4-yl)-2-(tetrahydro-2H-pyran-4-yloxy)benzonitrile;
5-(2-{[4-(1H-imidazol-1-ylmethyl)phenyl]amino}pyrimidin-4-yl)-2-(tetrahydro-2H-pyran-4-yloxy)benzonitrile;
5-(2-{[4-(hexahydropyrrolo[1,2-a]pyrazin-2(1H)-ylcarbonyl)-3-methoxyphenyl]amino}pyrimidin-4-yl)-2-(tetrahydro-2H-pyran-4-yloxy)benzonitrile;
5-(2-{[4-(1,3′-bipyrrolidin-1′-ylcarbonyl)-3-methoxyphenyl]amino}pyrimidin-4-yl)-2-(tetrahydro-2H-pyran-4-yloxy)benzonitrile;
5-{2-[(3-methoxy-4-{[4-(propan-2-yl)piperazin-1-yl]carbonyl}phenyl)amino]pyrimidin-4-yl}-2-(tetrahydro-2H-pyran-4-yloxy)benzonitrile;
4-({4-[3-cyano-4-(tetrahydro-2H-pyran-4-yloxy)phenyl]pyrimidin-2-yl}amino)-2-methoxy-N-[2-(pyrrolidin-1-yl)ethyl]benzamide;
4-({4-[3-cyano-4-(tetrahydro-2H-pyran-4-yloxy)phenyl]pyrimidin-2-yl}amino)-N-[2-(dimethylamino)ethyl]-2-methoxy-N-methylbenzamide;
4-({4-[3-cyano-4-(tetrahydro-2H-pyran-4-yloxy)phenyl]pyrimidin-2-yl}amino)-N-[2-(diethylamino)ethyl]-2-methoxybenzamide;
5-(2-{[4-({3-[(dimethylamino)methyl]azetidin-1-yl}carbonyl)-3-methoxyphenyl]amino}pyrimidin-4-yl)-2-(tetrahydro-2H-pyran-4-yloxy)benzonitrile;
5-(2-{[4-(morpholin-4-ylmethyl)phenyl]amino}pyrimidin-4-yl)-2-(tetrahydro-2H-pyran-4-yloxy)benzonitrile;
5-{2-[(4-{[4-(2-hydroxyethyl)piperazin-1-yl]methyl}phenyl)amino]pyrimidin-4-yl}-2-(tetrahydro-2H-pyran-4-yloxy)benzonitrile;
5-[2-({4-[4-(2-hydroxyethyl)piperazin-1-yl]phenyl}amino)pyrimidin-4-yl]-2-(tetrahydro-2H-pyran-4-yloxy)benzonitrile;
5-[2-({4-Methyl-3-[3-(morpholin-4-yl)propoxy]phenyl}amino)pyrimidin-4-yl]-2-(tetrahydro-2H-pyran-4-yloxy)benzonitrile;
5-[2-({3-[2-(Morpholin-4-yl)ethoxy]phenyl}amino)pyrimidin-4-yl]-2-(tetrahydro-2H-pyran-4-yloxy)benzonitrile;
5-[2-({4-Fluoro-3-[3-(morpholin-4-yl)propoxy]phenyl}amino)pyrimidin-4-yl]-2-(tetrahydro-2H-pyran-4-yloxy)benzonitrile;
5-{2-[(4-methoxy-3-{3-[1-(propan-2-yl)piperidin-4-yl]propoxy}phenyl)amino]pyrimidin-4-yl}-2-(tetrahydro-2H-pyran-4-yloxy)benzonitrile;
5-[2-({3-[3-(1-ethylpiperidin-4-yl)propoxy]-4-methoxyphenyl}amino)pyrimidin-4-yl]-2-(tetrahydro-2H-pyran-4-yloxy)benzonitrile;
5-[2-({4-methoxy-3-[3-(piperidin-4-yl)propoxy]phenyl}amino)pyrimidin-4-yl]-2-(tetrahydro-2H-pyran-4-yloxy)benzonitrile;
5-{2-[(4-methoxy-3-{3-[4-(propan-2-yl)piperazin-1-yl]propoxy}phenyl)amino]pyrimidin-4-yl}-2-(tetrahydro-2H-pyran-4-yloxy)benzonitrile;
5-{2-[(4-methoxy-3-{3-[4-(2-methylpropanoyl)piperazin-1-yl]propoxy}phenyl)amino]pyrimidin-4-yl}-2-(tetrahydro-2H-pyran-4-yloxy)benzonitrile;
5-[2-({3-[3-(4-ethylpiperazin-1-yl)propoxy]-4-methoxyphenyl}amino)pyrimidin-4-yl]-2-(tetrahydro-2H-pyran-4-yloxy)benzonitrile;
5-[2-({4-methoxy-3-[3-(piperazin-1-yl)propoxy]phenyl}amino)pyrimidin-4-yl]-2-(tetrahydro-2H-pyran-4-yloxy)benzonitrile;
5-[2-({4-[3-(morpholin-4-yl)propoxy]phenyl}amino)pyrimidin-4-yl]-2-(tetrahydro-2H-pyran-4-yloxy)benzonitrile;
5-[2-({4-methoxy-3-[3-(morpholin-4-yl)propoxy]phenyl}amino)pyrimidin-4-yl]-2-(tetrahydro-2H-pyran-4-yloxy)benzonitrile;
5-[2-({4-[2-(diethylamino)ethoxy]-3-methoxyphenyl}amino)pyrimidin-4-yl]-2-(tetrahydro-2H-pyran-4-yloxy)benzonitrile;
5-{2-[(3-{2-[2-(diethylamino)ethoxy]ethoxy}-4-methoxyphenyl)amino]pyrimidin-4-yl}-2-(tetrahydro-2H-pyran-4-yloxy)benzonitrile;
5-[2-({4-Methyl-3-[2-(piperazin-1-yl)ethoxy]phenyl}amino)pyrimidin-4-yl]-2-(tetrahydro-2H-pyran-4-yloxy)benzonitrile;
1-[3-({4-[3-Cyano-4-(2-methylpropoxy)phenyl]pyrimidin-2-yl}amino)phenyl]-N-(2-hydroxyethyl)methanesulfonamide;
2-(Cyclopropylmethoxy)-5-[2-({3-[2-(diethylamino)ethoxy]-4-fluorophenyl}amino)pyrimidin-4-yl]benzonitrile;
N-[3-({4-[3-Cyano-4-(tetrahydro-2H-pyran-4-yloxy)phenyl]pyrimidin-2-yl}amino)phenyl]-3-hydroxypyrrolidine-1-carboxamide;
N-[3-({4-[3-Cyano-4-(tetrahydro-2H-pyran-4-yloxy)phenyl]pyrimidin-2-yl}amino)phenyl]-3-methoxypropanamide;
5-(2-{[3-(Dimethylamino)phenyl]amino}pyrimidin-4-yl)-2-(tetrahydro-2H-pyran-4-yloxy)benzonitrile;
5-{2-[(3-{[2-(Dimethylamino)ethyl]amino}phenyl)amino]pyrimidin-4-yl}-2-(tetrahydro-2H-pyran-4-yloxy)benzonitrile;
5-(2-{[4-Fluoro-3-(pyrrolidin-3-yloxy)phenyl]amino}pyrimidin-4-yl)-2-(tetrahydro-2H-pyran-4-yloxy)benzonitrile;
5-(2-{[3-(Pyrrolidin-1-ylmethyl)phenyl]amino}pyrimidin-4-yl)-2-(tetrahydro-2H-pyran-4-yloxy)benzonitrile;
1-[3-({4-[3-Cyano-4-(tetrahydro-2H-pyran-4-yloxy)phenyl]pyrimidin-2-yl}amino)phenyl]-3-(2-methoxyethyl)urea;
5-{2-[(3-Ethylphenyl)amino]pyrimidin-4-yl}-2-{[(3R)-1-(hydroxyacetyl)pyrrolidin-3-yl]oxy}benzonitrile;
5-(2-{[4-Fluoro-3-(morpholin-3-ylmethoxy)phenyl]amino}pyrimidin-4-yl)-2-(tetrahydro-2H-pyran-4-yloxy)benzonitrile;
2-{[(3R)-1-(Hydroxyacetyl)pyrrolidin-3-yl]oxy}-5-(2-{[3-(3-methoxypyrrolidin-1-yl)phenyl]amino}pyrimidin-4-yl)benzonitrile;
N-[3-({4-[3-Cyano-4-(tetrahydro-2H-pyran-4-yloxy)phenyl]pyrimidin-2-yl}amino)phenyl]-1-methyl-1H-pyrazole-3-carboxamide;
5-[2-({3-[(Dimethylamino)methyl]phenyl}amino)pyrimidin-4-yl]-2-(tetrahydro-2H-pyran-4-yloxy)benzonitrile;
5-(2-{[3-(Pyridin-3-yl)phenyl]amino}pyrimidin-4-yl)-2-(tetrahydro-2H-pyran-4-yloxy)benzonitrile;
5-(2-{[4-(Pyridin-3-yl)phenyl]amino}pyrimidin-4-yl)-2-(tetrahydro-2H-pyran-4-yloxy)benzonitrile;
5-(5-Fluoro-2-{[3-methoxy-4-(morpholin-4-yl)phenyl]amino}pyrimidin-4-yl)-2-{[(3R)-1-(hydroxyacetyl)pyrrolidin-3-yl]oxy}benzonitrile;
4-[(4-{3-Cyano-4-[(cyclopropylcarbonyl)amino]phenyl}pyrimidin-2-yl)amino]-2-methoxy-N-(2-methoxyethyl)benzamide;
5-(2-{[3-(2-Aminoethoxy)-4-methylphenyl]amino}pyrimidin-4-yl)-2-(tetrahydro-2H-pyran-4-yloxy)benzonitrile;
5-(2-{[3-(1H-Imidazol-1-yl)phenyl]amino}pyrimidin-4-yl)-2-(tetrahydro-2H-pyran-4-yloxy)benzonitrile;
5-[2-({3-[(3-Hydroxypyrrolidin-1-yl)methyl]phenyl}amino)pyrimidin-4-yl]-2-(tetrahydro-2H-pyran-4-yloxy)benzonitrile;
N-[3-({4-[3-Cyano-4-(tetrahydro-2H-pyran-4-yloxy)phenyl]pyrimidin-2-yl}amino)phenyl]-2-hydroxy-2-methylpropanamide;
4-({4-[3-Cyano-4-(tetrahydro-2H-pyran-4-yloxy)phenyl]pyrimidin-2-yl}amino)benzenesulfonamide;
4-({4-[3-Cyano-4-(2-methylpropoxy)phenyl]pyrimidin-2-yl}amino)-N-(2-methoxyethyl)benzamide;
N-(2-Cyano-4-{2-[(4-{[(2-hydroxyethyl)sulfamoyl]methyl}phenyl)amino]pyrimidin-4-yl}phenyl)cyclopropanecarboxamide;
5-(2-{[4-(Azetidin-1-ylcarbonyl)-3-methoxyphenyl]amino}pyrimidin-4-yl)-2-(tetrahydro-2H-pyran-4-yloxy)benzonitrile;
5-[2-({4-[1-(3-Methoxyazetidin-1-yl)ethyl]phenyl}amino)pyrimidin-4-yl]-2-(tetrahydro-2H-pyran-4-yloxy)benzonitrile;
5-(2-{[3-(3-Methoxyazetidin-1-yl)-4-methylphenyl]amino}pyrimidin-4-yl)-2-(tetrahydro-2H-pyran-4-yloxy)benzonitrile;
5-(2-{[3-(Pyridin-4-yl)phenyl]amino}pyrimidin-4-yl)-2-(tetrahydro-2H-pyran-4-yloxy)benzonitrile;
2-(Cyclopropylmethoxy)-5-{2-[(4-fluoro-3-{2-[4-(propan-2-yl)piperazin-1-yl]ethoxy}phenyl)amino]pyrimidin-4-yl}benzonitrile;
4-({4-[3-Cyano-4-(tetrahydro-2H-pyran-4-yloxy)phenyl]pyrimidin-2-yl}amino)-N-(1,3-thiazol-2-yl)benzenesulfonamide;
2-(Tetrahydro-2H-pyran-4-yloxy)-5-(2-{[3-(1H-1,2,3-triazol-1-ylmethyl)phenyl]amino}pyrimidin-4-yl)benzonitrile;
5-[2-({3-[2-(Diethylamino)ethoxy]-4-fluorophenyl}amino)pyrimidin-4-yl]-2-({1-[(2S)-2-hydroxypropanoyl]piperidin-4-yl}oxy)benzonitrile;
5-(2-{[3-(1H-Pyrazol-1-yl)phenyl]amino}pyrimidin-4-yl)-2-(tetrahydro-2H-pyran-4-yloxy)benzonitrile;
5-(2-{[4-(1H-Pyrazol-4-yl)phenyl]amino}pyrimidin-4-yl)-2-(tetrahydro-2H-pyran-4-yloxy)benzonitrile;
2-(Tetrahydro-2H-pyran-4-yloxy)-5-(2-{[4-(1H-1,2,4-triazol-1-yl)phenyl]amino}pyrimidin-4-yl)benzonitrile;
2-(Cyclopropylmethoxy)-5-{2-[(4-{[(2-methoxyethyl)amino]methyl}phenyl)amino]pyrimidin-4-yl}benzonitrile;
5-[2-(1H-Benzimidazol-5-ylamino)pyrimidin-4-yl]-2-(tetrahydro-2H-pyran-4-yloxy)benzonitrile;
5-(2-{[4-(1-Methyl-1H-pyrazol-4-yl)phenyl]amino}pyrimidin-4-yl)-2-(tetrahydro-2H-pyran-4-yloxy)benzonitrile;
5-(2-{[3-(Morpholin-4-yl)phenyl]amino}pyrimidin-4-yl)-2-(tetrahydro-2H-pyran-4-yloxy)benzonitrile;
5-[2-({3-[2-(Diethylamino)ethoxy]-4-fluorophenyl}amino)pyrimidin-4-yl]-2-(tetrahydro-2H-pyran-4-yloxy)benzonitrile;
5-[2-({3-Methoxy-4-[(3-methoxyazetidin-1-yl)carbonyl]phenyl}amino)pyrimidin-4-yl]-2-(2-methylpropoxy)benzonitrile;
2-{[(3R)-1-(Hydroxyacetyl)pyrrolidin-3-yl]oxy}-5-(2-{[3-methoxy-4-(morpholin-4-yl)phenyl]amino}pyrimidin-4-yl)benzonitrile;
1-[3-({4-[3-Cyano-4-(tetrahydro-2H-pyran-4-yloxy)phenyl]pyrimidin-2-yl}amino)phenyl]-3-(4-hydroxycyclohexyl)urea;
5-(2-{[4-Methyl-3-(morpholin-4-yl)phenyl]amino}pyrimidin-4-yl)-2-(tetrahydro-2H-pyran-4-yloxy)benzonitrile;
5-[2-({3-[3-(Dimethylamino)pyrrolidin-1-yl]phenyl}amino)pyrimidin-4-yl]-2-(tetrahydro-2H-pyran-4-yloxy)benzonitrile;
5-(5-Fluoro-2-{[4-(morpholin-4-yl)phenyl]amino}pyrimidin-4-yl)-2-(tetrahydro-2H-pyran-4-yloxy)benzonitrile;
4-({4-[3-Cyano-4-(tetrahydro-2H-pyran-4-yloxy)phenyl]pyrimidin-2-yl}amino)-N-(pyridin-2-yl)benzenesulfonamide;
2-(Tetrahydro-2H-pyran-4-yloxy)-5-(2-{[3-(1H-tetrazol-5-yl)phenyl]amino}pyrimidin-4-yl)benzonitrile;
2-(Tetrahydro-2H-pyran-4-yloxy)-5-(2-{[3-(4H-1,2,4-triazol-4-ylmethyl)phenyl]amino}pyrimidin-4-yl)benzonitrile;
5-[2-({3-[3-(2-Methoxyethoxy)azetidin-1-yl]-4-methylphenyl}amino)pyrimidin-4-yl]-2-(tetrahydro-2H-pyran-4-yloxy)benzonitrile;
5-{2-[(4-Methyl-3-{2-[4-(propan-2-yl)piperazin-1-yl]ethoxy}phenyl)amino]pyrimidin-4-yl}-2-(tetrahydro-2H-pyran-4-yloxy)benzonitrile;
N-[3-({4-[3-Cyano-4-(tetrahydro-2H-pyran-4-yloxy)phenyl]pyrimidin-2-yl}amino)phenyl]-3-hydroxyazetidine-1-carboxamide;
5-[2-({4-[(3-Ethoxyazetidin-1-yl)carbonyl]-3-methoxyphenyl}amino)pyrimidin-4-yl]-2-(tetrahydro-2H-pyran-4-yloxy)benzonitrile;
1-[3-({4-[3-Cyano-4-(tetrahydro-2H-pyran-4-yloxy)phenyl]pyrimidin-2-yl}amino)phenyl]-N,N-dimethylmethanesulfonamide;
N-{2-Cyano-4-[2-({3-methoxy-4-[(3-methoxyazetidin-1-yl)carbonyl]phenyl}amino)pyrimidin-4-yl]phenyl}cyclopropanecarboxamide;
2-{[(3R)-1-(Hydroxyacetyl)pyrrolidin-3-yl]oxy}-5-[2-({3-[4-(2-hydroxyethyl)piperazin-1-yl]phenyl}amino)pyrimidin-4-yl]benzonitrile;
1-[4-({4-[3-Cyano-4-(2-methylpropoxy)phenyl]pyrimidin-2-yl}amino)phenyl]-N-methylmethanesulfonamide;
2-(Tetrahydro-2H-pyran-4-yloxy)-5-(2-{[4-(4H-1,2,4-triazol-4-yl)phenyl]amino}pyrimidin-4-yl)benzonitrile;
5-(2-{[3-(2,3-Dihydroxypropoxy)-4-fluorophenyl]amino}pyrimidin-4-yl)-2-(tetrahydro-2H-pyran-4-yloxy)benzonitrile;
5-[2-({4-[(2-Methyl-1H-imidazol-1-yl)methyl]phenyl}amino)pyrimidin-4-yl]-2-(tetrahydro-2H-pyran-4-yloxy)benzonitrile;
5-(2-{[4-(Pyridin-4-yl)phenyl]amino}pyrimidin-4-yl)-2-(tetrahydro-2H-pyran-4-yloxy)benzonitrile;
1-[3-({4-[3-Cyano-4-(cyclopropylmethoxy)phenyl]pyrimidin-2-yl}amino)phenyl]-N-(2-hydroxyethyl)methanesulfonamide;
5-(2-{[3-(2-Aminoethoxy)-4-fluorophenyl]amino}pyrimidin-4-yl)-2-(cyclopropylmethoxy)benzonitrile;
5-(2-{[3-Methoxy-4-(pyrrolidin-1-ylcarbonyl)phenyl]amino}pyrimidin-4-yl)-2-(tetrahydro-2H-pyran-4-yloxy)benzonitrile;
5-[2-({4-[(1E)-3-(Morpholin-4-yl)prop-1-en-1-yl]phenyl}amino)pyrimidin-4-yl]-2-(tetrahydro-2H-pyran-4-yloxy)benzonitrile;
2-{[(3R)-1-(Hydroxyacetyl)pyrrolidin-3-yl]oxy}-5-[2-({4-[(3-hydroxyazetidin-1-yl)methyl]phenyl}amino)pyrimidin-4-yl]benzonitrile;
5-{2-[(3-{[2-(4-Methylpiperazin-1-yl)ethyl]amino}phenyl)amino]pyrimidin-4-yl}-2-(tetrahydro-2H-pyran-4-yloxy)benzonitrile;
2-(Cyclopropylmethoxy)-5-[2-({3-methoxy-4-[(3-methoxyazetidin-1-yl)methyl]phenyl}amino)pyrimidin-4-yl]benzonitrile;
5-[2-({3-[2-(Diethylamino)ethoxy]-4-methylphenyl}amino)pyrimidin-4-yl]-2-(tetrahydro-2H-pyran-4-yloxy)benzonitrile;
1-[3-({4-[3-Cyano-4-(tetrahydro-2H-pyran-4-yloxy)phenyl]pyrimidin-2-yl}amino)phenyl]-N-(2-hydroxyethyl)methanesulfonamide;
5-[2-({3-[4-(2-Hydroxyethyl)piperazin-1-yl]phenyl}amino)pyrimidin-4-yl]-2-(tetrahydro-2H-pyran-4-yloxy)benzonitrile;
2-(Cyclopropylmethoxy)-5-[2-({3-methoxy-4-[(3-methoxyazetidin-1-yl)carbonyl]phenyl}amino)pyrimidin-4-yl]benzonitrile;
1-[3-({4-[3-Cyano-4-(tetrahydro-2H-pyran-4-yloxy)phenyl]pyrimidin-2-yl}amino)phenyl]-3-(2-hydroxyethyl)urea;
2-(Tetrahydro-2H-pyran-4-yloxy)-5-(2-{[4-(1H-1,2,4-triazol-1-ylmethyl)phenyl]amino}pyrimidin-4-yl)benzonitrile;
5-{2-[(3-{[4-(2-Hydroxyethyl)piperazin-1-yl]methyl}phenyl)amino]pyrimidin-4-yl}-2-(tetrahydro-2H-pyran-4-yloxy)benzonitrile;
5-[2-({4-Fluoro-3-[2-(piperazin-1-yl)ethoxy]phenyl}amino)pyrimidin-4-yl]-2-(tetrahydro-2H-pyran-4-yloxy)benzonitrile;
N-(2-Cyano-4-{2-[(3-{[(2-hydroxyethyl)sulfamoyl]methyl}phenyl)amino]pyrimidin-4-yl}phenyl)cyclopropanecarboxamide;
5-{2-[(3-{[2-(Dimethylamino)ethyl]amino}-4-methylphenyl)amino]pyrimidin-4-yl}-2-(tetrahydro-2H-pyran-4-yloxy)benzonitrile;
2-(Tetrahydro-2H-pyran-4-yloxy)-5-(2-{[4-(1H-tetrazol-1-ylmethyl)phenyl]amino}pyrimidin-4-yl)benzonitrile;
N-{[4-({4-[3-Cyano-4-(tetrahydro-2H-pyran-4-yloxy)phenyl]pyrimidin-2-yl}amino)phenyl]sulfonyl}acetamide;
3-[3-({4-[3-Cyano-4-(tetrahydro-2H-pyran-4-yloxy)phenyl]pyrimidin-2-yl}amino)phenyl]-1,1-dimethylurea;
5-{2-[(3-Methoxy-4-{[3-(2-methoxyethoxy)azetidin-1-yl]carbonyl}phenyl)amino]pyrimidin-4-yl}-2-(tetrahydro-2H-pyran-4-yloxy)benzonitrile;
4-({4-[3-Cyano-4-(tetrahydro-2H-pyran-4-yloxy)phenyl]pyrimidin-2-yl}amino)-N-(4-methylpyrimidin-2-yl)benzenesulfonamide;
2-{[(3R)-1-(Hydroxyacetyl)pyrrolidin-3-yl]oxy}-5-{2-[(4-{[4-(2-hydroxyethyl)piperazin-1-yl]methyl}phenyl)amino]pyrimidin-4-yl}benzonitrile;
1-[4-({4-[3-Cyano-4-(cyclopropylmethoxy)phenyl]pyrimidin-2-yl}amino)phenyl]-N-(2-hydroxyethyl)methanesulfonamide;
5-(2-{[3-(Morpholin-4-ylmethyl)phenyl]amino}pyrimidin-4-yl)-2-(tetrahydro-2H-pyran-4-yloxy)benzonitrile;
2-{[(3R)-1-(Hydroxyacetyl)pyrrolidin-3-yl]oxy}-5-(2-{[3-(3-methoxyazetidin-1-yl)phenyl]amino}pyrimidin-4-yl)benzonitrile;
5-(2-{[3-(2-Aminoethoxy)-4-fluorophenyl]amino}pyrimidin-4-yl)-2-(tetrahydro-2H-pyran-4-yloxy)benzonitrile;
5-[2-({3-[(Dimethylamino)methyl]phenyl}amino)pyrimidin-4-yl]-2-{[(3R)-1-(hydroxyacetyl)pyrrolidin-3-yl]oxy}benzonitrile;
5-{2-[(3,4-Dimethylphenyl)amino]pyrimidin-4-yl}-2-{[(3R)-1-(hydroxyacetyl)pyrrolidin-3-yl]oxy}benzonitrile;
1-[4-({4-[3-Cyano-4-(cyclopropylmethoxy)phenyl]pyrimidin-2-yl}amino)phenyl]-N-methylmethanesulfonamide;
1-[4-({4-[3-Cyano-4-(2-methylpropoxy)phenyl]pyrimidin-2-yl}amino)phenyl]-N-(2-hydroxyethyl)methanesulfonamide;
N-[3-({4-[3-Cyano-4-(tetrahydro-2H-pyran-4-yloxy)phenyl]pyrimidin-2-yl}amino)phenyl]morpholine-4-carboxamide;
N-[3-({4-[3-Cyano-4-(tetrahydro-2H-pyran-4-yloxy)phenyl]pyrimidin-2-yl}amino)phenyl]-2-methoxyacetamide;
1-[3-({4-[3-Cyano-4-(tetrahydro-2H-pyran-4-yloxy)phenyl]pyrimidin-2-yl}amino)phenyl]-N-methylmethanesulfonamide;
1-[3-({4-[3-Cyano-4-(tetrahydro-2H-pyran-4-yloxy)phenyl]pyrimidin-2-yl}amino)phenyl]-3-(2-hydroxy-2-methylpropyl)urea;
5-{2-[(4-Fluoro-3-{2-[4-(propan-2-yl)piperazin-1-yl]ethoxy}phenyl)amino]pyrimidin-4-yl}-2-(tetrahydro-2H-pyran-4-yloxy)benzonitrile;
5-{2-[(4-{[(2-Methoxyethyl)amino]methyl}phenyl)amino]pyrimidin-4-yl}-2-(2-methylpropoxy)benzonitrile;
5-[2-({3-[(4-Methyl-1H-imidazol-1-yl)methyl]phenyl}amino)pyrimidin-4-yl]-2-(tetrahydro-2H-pyran-4-yloxy)benzonitrile;
2-(Cyclopropylmethoxy)-5-[2-({4-fluoro-3-[2-(piperazin-1-yl)ethoxy]phenyl}amino)pyrimidin-4-yl]benzonitrile;
5-(2-{[3-(2-Aminoethoxy)-4-fluorophenyl]amino}pyrimidin-4-yl)-2-({1-[(2S)-2-hydroxypropanoyl]piperidin-4-yl}oxy)benzonitrile;
N-[3-({4-[3-Cyano-4-(tetrahydro-2H-pyran-4-yloxy)phenyl]pyrimidin-2-yl}amino)phenyl]acetamide;
5-{2-[(3-{[2-(Morpholin-4-yl)ethyl]amino}phenyl)amino]pyrimidin-4-yl}-2-(tetrahydro-2H-pyran-4-yloxy)benzonitrile;
2-{[(3R)-1-(Hydroxyacetyl)pyrrolidin-3-yl]oxy}-5-[2-({4-[(3-methoxyazetidin-1-yl)methyl]phenyl}amino)pyrimidin-4-yl]benzonitrile;
(2R)—N-[3-({4-[3-Cyano-4-(tetrahydro-2H-pyran-4-yloxy)phenyl]pyrimidin-2-yl}amino)phenyl]-2-hydroxypropanamide;
5-{2-[(3-{[2-(Dimethylamino)ethyl](methyl)amino}phenyl)amino]pyrimidin-4-yl}-2-(tetrahydro-2H-pyran-4-yloxy)benzonitrile;
2-{[(3R)-1-(Hydroxyacetyl)pyrrolidin-3-yl]oxy}-5-[2-({3-[(4-methyl-1H-imidazol-1-yl)methyl]phenyl}amino)pyrimidin-4-yl]benzonitrile;
5-(2-{[3-Methoxy-4-(1H-tetrazol-1-yl)phenyl]amino}pyrimidin-4-yl)-2-(tetrahydro-2H-pyran-4-yloxy)benzonitrile;
N-{2-Cyano-4-[2-({4-[(3-methoxyazetidin-1-yl)carbonyl]phenyl}amino)pyrimidin-4-yl]phenyl}cyclopropanecarboxamide;
4-({4-[3-Cyano-4-(cyclopropylmethoxy)phenyl]pyrimidin-2-yl}amino)-N-(2-methoxyethyl)benzamide;
N-[3-({4-[3-Cyano-4-(tetrahydro-2H-pyran-4-yloxy)phenyl]pyrimidin-2-yl}amino)phenyl]-3-(dimethylamino)pyrrolidine-1-carboxamide;
N-[3-({4-[3-Cyano-4-(tetrahydro-2H-pyran-4-yloxy)phenyl]pyrimidin-2-yl}amino)phenyl]-3-methoxyazetidine-1-carboxamide;
2-{[(3R)-1-(Hydroxyacetyl)pyrrolidin-3-yl]oxy}-5-[2-({3-[(2S)-2-(hydroxymethyl)pyrrolidin-1-yl]phenyl}amino)pyrimidin-4-yl]benzonitrile; and
2-(Cyclopropylmethoxy)-5-(2-{[4-fluoro-3-(pyrrolidin-3-yloxy)phenyl]amino}pyrimidin-4-yl)benzonitrile.

Further description of exemplary compounds according to Formula I is provided in the Examples section below, in the form of the several hundred specific example compounds made by the synthetic schemes disclosed.

For therapeutic use, salts of the compounds according to Formula I are those wherein the counterion is pharmaceutically acceptable. However, salts of acids and bases which are non-pharmaceutically acceptable may also find use, for example, in the preparation or purification of a pharmaceutically acceptable compound.

The pharmaceutically acceptable addition salts as mentioned herein are meant to comprise the therapeutically active non-toxic acid addition salt forms which the compounds according to Formula I are able to form. The latter can be obtained by treating the base form with such appropriate acids as inorganic acids, for example, hydrohalic acids, e.g. hydrochloric, hydrobromic and the like; sulfuric acid; nitric acid; phosphoric acid and the like; or organic acids, for example, acetic, propanoic, hydroxy-acetic, 2-hydroxypropanoic, 2-oxopropanoic, oxalic, malonic, succinic, maleic, fumaric, malic, tartaric, 2-hydroxy-1,2,3-propanetricarboxylic, methanesulfonic, ethanesulfonic, benzenesulfonic, 4-methylbenzenesulfonic, cyclohexanesulfamic, 2-hydroxybenzoic, 4-amino-2-hydroxybenzoic and the like acids. Conversely the salt form can be converted by treatment with alkali into the free base form.

The compounds according to Formula I containing acidic protons may be converted into their therapeutically active non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline, the benzathine, N-methyl-D-glucamine, 2-amino-2-(hydroxymethyl)-1,3-propanedi-ol, hydrabamine salts, and salts with amino acids such as, for example, arginine, lysine and the like. Conversely, the salt form can be converted by treatment with acid into the free acid form.

The term addition salt also comprises the hydrates and solvent addition forms which the compounds according to Formula I are able to form. Examples of such forms are e.g. hydrates, alcoholates and the like.

The term “quaternary amine” as used herein defines the quaternary ammonium salts which the compounds according to Formula I are able to form by reaction between a basic nitrogen of a compound according to Formula I and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen. Pharmaceutically acceptable counterions include chloro, bromo, iodo, trifluoroacetate and acetate, among others. The counterion of choice can be introduced using ion exchange resins.

Pharmaceutically acceptable salts of the compound of the present invention include all salts and are exemplified by alkaline salts with an inorganic acid or a salt with an organic acid that are known in the art. In addition, pharmaceutically acceptable salts include acid salts of inorganic bases, as well as acid salts of organic bases. Their hydrates, solvates, and the like are also encompassed in the present invention. In addition, N-oxide compounds are also encompassed in the present invention.

It will be appreciated that some of the compounds according to Formula I and their N-oxides, addition salts, quaternary amines and stereochemically isomeric forms may contain one or more centers of chirality and exist as stereochemically isomeric forms.

The term “stereochemically isomeric forms” as used hereinbefore defines all possible stereoisomeric forms which the compounds according to Formula I, and their N-oxides, addition salts, quaternary amines or physiologically functional derivatives may possess. Unless otherwise mentioned or indicated, the chemical designation of compounds denotes the mixture of all possible stereochemically isomeric forms, said mixtures containing all diastereomers and enantiomers of the basic molecular structure as well as each of the individual isomeric forms of the compounds according to Formula I and their N-oxides, salts, solvates or quaternary amines substantially free, i.e. associated with less than about 10%, less than about 5%, less than about 2% and less than about 1% of the other isomers. Stereogenic centers may have the R- or S-configuration; substituents on bivalent cyclic (partially) saturated radicals may have either the cis- or trans-configuration. Compounds encompassing double bonds can have an E- or Z-stereochemistry at said double bond. Stereochemically isomeric forms of the compounds according to Formula I are fully intended to be embraced within the scope of the present invention.

The N-oxide forms of the compounds according to Formula I are meant to comprise the compounds according to Formula I wherein one or several nitrogen atoms are oxidized to the so-called N-oxide.

Some of the compounds according to Formula I may also exist in their tautomeric form. Such forms, although not explicitly indicated in the above formulae, are intended to be included within the scope of the present invention.

Whenever used hereinafter, the term “compounds according to Formula I” is meant to also include the N-oxide forms, salts, and quaternary amines, as well as the stereochemically isomeric forms of the compound according to Formula I. Of particular interest are those compounds according to Formula I that are stereochemically pure.

Some compounds according to Formula I are provided having an IC₅₀, as determined in the in-vitro IKKε kinase inhibition assays as described below (i.e., In-Vitro IKKε and TBK1 Kinase Assays), ranging from about 490 nM to about 50 nM. Other compounds according to Formula I are provided having an IC₅₀, as determined in the in-vitro IKKε kinase inhibition assays as described below, ranging from about 50 nM to about 5 nM. Other compounds according to Formula I are provided having an IC₅₀, as determined in the in-vitro IKKε kinase inhibition assays as described below, of less than about 5 nM.

It is believed that compounds according to Formula I and having an IKKε kinase inhibitory activity (IC₅₀ value) of less than about 0.005 μM (5 nM), as determined in the in-vitro IKKε kinase inhibition assays as described below, are sufficiently active for the uses disclosed hereinafter. These compounds include, for example, Example Compounds 2, 3, 4, 5, 6, 11, 14, 15, 16, 18, 20, 21, 22, 24, 25, 26, 27, 28, 29, 30, 31, 32, 33, 34, 35, 59, 68, 72, 73, 75, 76, 80, 82, 83, 88, 91, 93, 96, 98, 100, 103, 104, 107, 111, 114, 115, 118, 124, 127, 129, 130, 132, 134, 155, 157, 158, 164, 165, 171, 176, 178, 181, 184, 190, 191, 206, 208, 210, 211, 212, 216, 223, 225, 231, 235, 237, 239, 242, 246, 253, 256, 261, 262, 264, 271, 275, 287, 290, 307, 311, 326, 329, 331, 334, 335, 341, 354, 367, 370, 371, 373, 374, 376, 377, 381, 385, 392, 393, 394, 395, 396, 397, 400, 401, 402, 403, 404, 405, 406, 413, 415, 436, 437, 438, 439, 440, 442, 444, 446, 467, 471, 475, 476, 477, 478, 479, 480, 481, 482, 484, 485, 486, 487, 488, 489, 490, 492, 493, 494, 495, 496, 497, 498, 500, 501, 502, 503, 504, 505, 506, 507, 510, 511, 512, 517, 518, 519, 520, 521, 522, 523, 524, 525, 526, 527, 529, 530, 531, 533, 534, 535, 536, 538, 539, 540, 541, 542, 543, 544, 545, 546, 547, 548, 549, 550, 552, 558, 559, 560, 561, 563, 564, 565, 566, 567, 571, 572, 573, 574, 575, 576, 577, 578, 579, 580, 581, 583, 584, 585, 586, 587, 588, 589, 590, 591, 592, 593, 594, 595, 596, 597, 598, 599, 601, 603, 604, 606, 607, 608, 609, 610, 611, 612, 613, 614, 615, 616, 617, 618, 619, 620, 621, 622, 624, 625, 626, 627, 628, 629, 630, 631, 632, 635, 636, 637, 638, 639, 640, 642, 643, 644, 645, 646, 647, 648, 649, 650, 651, 653, 654, 655, 656, 657, 658, 659, 661, 662, 664, 665, 666, 667, 668, 669, and 670, as identified below.

It should also be understood that in the compounds according to Formula I, reference to any bound hydrogen atom may also encompass a deuterium atom bound at the same position. Substitution of hydrogen atoms with deuterium atoms is conventional in the art. See, e.g., U.S. Pat. Nos. 5,149,820 & 7,317,039. Such deuteration sometimes results in a compound that is functionally indistinct from its hydrogenated counterpart, but occasionally results in a compound having beneficial changes in the properties relative to the non-deuterated form. For example, in certain instances, replacement of specific bound hydrogen atoms with deuterium atoms dramatically slows the catabolism of the deuterated compound, relative to the non-deuterated compound, such that the deuterated compound exhibits a longer half-life in the bodies of individuals administered such compounds. This is particularly the case when the catabolism of the hydrogenated compound is mediated by cytochrome P450 systems. See Kushner et al., Can. J. Physiol. Pharmacol. (1999) 77:79-88.

### 3. Pharmaceutical Compositions and Formulations

The present invention also provides medicaments or pharmaceutical compositions comprising a therapeutically or prophylactically effective amount of at least one compound according to the present invention (i.e., at least one compound according to Formula I). Particularly, the present invention also provides medicaments or pharmaceutical compositions comprising a therapeutically or prophylactically effective amount of at least one compound according to the present invention having an IKKε kinase inhibitory activity (IC50 value) of less than about 0.005 μM (5 nM), as determined in the in-vitro IKKε kinase inhibition assays as described below. These compounds include, for example, Example Compounds 2, 3, 4, 5, 6, 11, 14, 15, 16, 18, 20, 21, 22, 24, 25, 26, 27, 28, 29, 30, 31, 32, 33, 34, 35, 59, 68, 72, 73, 75, 76, 80, 82, 83, 88, 91, 93, 96, 98, 100, 103, 104, 107, 111, 114, 115, 118, 124, 127, 129, 130, 132, 134, 155, 157, 158, 164, 165, 171, 176, 178, 181, 184, 190, 191, 206, 208, 210, 211, 212, 216, 223, 225, 231, 235, 237, 239, 242, 246, 253, 256, 261, 262, 264, 271, 275, 287, 290, 307, 311, 326, 329, 331, 334, 335, 341, 354, 367, 370, 371, 373, 374, 376, 377, 381, 385, 392, 393, 394, 395, 396, 397, 400, 401, 402, 403, 404, 405, 406, 413, 415, 436, 437, 438, 439, 440, 442, 444, 446, 467, 471, 475, 476, 477, 478, 479, 480, 481, 482, 484, 485, 486, 487, 488, 489, 490, 492, 493, 494, 495, 496, 497, 498, 500, 501, 502, 503, 504, 505, 506, 507, 510, 511, 512, 517, 518, 519, 520, 521, 522, 523, 524, 525, 526, 527, 529, 530, 531, 533, 534, 535, 536, 538, 539, 540, 541, 542, 543, 544, 545, 546, 547, 548, 549, 550, 552, 558, 559, 560, 561, 563, 564, 565, 566, 567, 571, 572, 573, 574, 575, 576, 577, 578, 579, 580, 581, 583, 584, 585, 586, 587, 588, 589, 590, 591, 592, 593, 594, 595, 596, 597, 598, 599, 601, 603, 604, 606, 607, 608, 609, 610, 611, 612, 613, 614, 615, 616, 617, 618, 619, 620, 621, 622, 624, 625, 626, 627, 628, 629, 630, 631, 632, 635, 636, 637, 638, 639, 640, 642, 643, 644, 645, 646, 647, 648, 649, 650, 651, 653, 654, 655, 656, 657, 658, 659, 661, 662, 664, 665, 666, 667, 668, 669, and 670, as identified below.

Typically, therapeutic compounds, such as the compounds according to Formula I, may be effective at an amount ranging from about 0.01 μg/kg to about 100 mg/kg per day based on total body weight of a human patient. The effective amount of a therapeutic compound in such a medicament or pharmaceutical formulation may be administered all at once and at one time, or may be divided into a number of smaller doses that are administered at predetermined intervals of time, or predetermined times of the day, for a specific duration of time or a specified number of days. The suitable dosage unit containing the effective amount of a therapeutic compound may, for each administration, range in total mass from about 1 μg to about 2000 mg, or may range from about 5 μg to about 1000 mg.

In the case of combination therapy, a therapeutically effective amount of one or more other therapeutically effective compounds can be administered in a separate pharmaceutical composition, or alternatively can be included in the pharmaceutical composition according to the present invention along with at least one compound according to Formula I. The pharmacology and toxicology of many of such other therapeutically effective compounds are known in the art. See e.g., Physicians Desk Reference, Medical Economics, Montvale, N.J.; and The Merck Index, Merck & Co., Rahway, N.J. The therapeutically effective amounts and suitable unit dosage ranges of such other therapeutically effective compounds used in art can be equally applicable in the present invention.

It should be understood that the dosage ranges set forth above are exemplary and are not intended to limit the scope of the present invention. The therapeutically effective amount for each therapeutically effective compound may vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration of therapeutically effective compounds may be adjusted as the various factors change over time.

In the pharmaceutical compositions of the present invention, the one or more compounds according to Formula I can be in any pharmaceutically acceptable salt form, as described above.

For oral administration, the one or more compounds according to Formula I may be incorporated into a pharmaceutical formulation that includes one or more pharmaceutically acceptable excipients or carriers such as binders, lubricants, disintegrating agents, and sweetening or flavoring agents, as known in the art. The formulation can be incorporated into enclosed gelatin capsules or compressed tablets. Capsules and tablets can be prepared using conventional techniques. The capsules and tablets may also be coated with various coatings known in the art to modify the flavors, tastes, colors, and shapes of the capsules and tablets. In addition, liquid carriers such as fatty oil may also be included in capsules.

Suitable oral formulations can also be in the form of suspensions, syrups, chewing gum, wafers, elixirs, and the like. If desired, conventional agents for modifying flavors, tastes, colors, and shapes of the various forms may also be included.

The compounds according to Formula I can also be administered parenterally in the form of a preformed solution or suspension, or a solution or suspension prepared from a lyophilized form before use. In such formulations, pharmaceutically acceptable diluents or pharmaceutically acceptable carriers such as sterile water, saline and buffered saline can be used. Other conventional and pharmaceutically acceptable solvents, pH buffers, stabilizers, anti-bacterial agents, surfactants, and antioxidants can be included. The parenteral formulations may be stored in conventional containers such as vials and ampoules that may be sized for preparing or delivering single doses of the formulation.

Routes of topical administration include nasal, bucal, mucosal, rectal, or vaginal applications. For topical administration, the active compounds may be formulated into lotions, creams, ointments, gels, powders, pastes, sprays, suspensions, drops and aerosols. Thus, one or more thickening agents, humectants, and stabilizing agents may be included in the formulations. One form of topical administration is delivery by a transdermal patch. Methods for preparing transdermal patches are disclosed, e.g., in Brown, et al.; Annual Review of Medicine, 39:221-229, 1988.

Subcutaneous implantation for sustained release of the one or more compounds according to Formula I may also be a suitable route of administration. This entails surgical procedures for implanting an active compound in any suitable formulation into a subcutaneous space, e.g., beneath the anterior abdominal wall. See, e.g., Wilson et al.; J. Clin. Psych., 45:242-247, 1984. Hydrogels may be used as a carrier for the sustained release of the active compounds. Hydrogels are generally known in the art. They are typically made by crosslinking high molecular weight biocompatible polymers into a network, which swells in water to form a gel like material. For the therapeutic methods of the present invention, hydrogels that are biodegradable or biosorbable are preferred. See, e.g., Phillips et al.; J. Pharmaceut. Sci., 73:1718-1720, 1984.

The compounds according to Formula I may also be conjugated to a water soluble non-immunogenic, non-peptidic, high molecular weight polymer to form a polymer conjugate. For example, one or more compounds according to Formula I may be covalently linked to polyethylene glycol to form a conjugate. Typically, such a conjugate exhibits improved solubility, stability, and reduced toxicity and immunogenicity. Thus, when administered to a patient, the one or more compounds according to Formula I in the conjugate can have a longer half-life in the body, and exhibit better efficacy. See generally, Burnham; Am. J. Hosp. Pharm., 15:210-218, 1994. PEGylated proteins are currently being used in protein replacement therapies and for other therapeutic uses. For example, PEGylated interferon (PEG-INTRON A®) is clinically used for treating Hepatitis B. PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL). In some embodiments of the present invention the covalent linkage between the polymer and the therapeutic compound or the polymer itself is hydrolytically degradable under physiological conditions. Such conjugates represent a type of “prodrug” that may readily release the active compound inside the body. Controlled release of an active compound may also be achieved by incorporating the active ingredient into microcapsules, nanocapsules, or hydrogels, as generally known in the art.

Liposomes may also be used as carriers for the compounds according to Formula I. Liposomes are micelles made of various lipids such as cholesterol, phospholipids, fatty acids, and derivatives thereof. Various modified lipids can also be used. Liposomes can reduce the toxicity of the active compounds, and increase their stability. Methods for preparing liposomal suspensions containing active ingredients therein are generally known in the art. See, e.g., U.S. Pat. No. 4,522,811; Prescott, Ed., Methods in Cell Biology, Volume XIV, Academic Press, New York, N.Y., 1976.

The one or more compounds according to Formula I may also be administered in combination with one or more other therapeutic compounds that synergistically treats or prevents the same symptoms or is effective for another disease or symptom for which the patient is being treated, so long as the one or more other therapeutic compounds does not interfere with, or adversely affect, the effects of the compounds according to Formula I. Such other therapeutic compounds include, but are not limited to, anti-inflammation agents, antiviral agents, antibiotics, antifungal agents, antithrombotic agents, cardiovascular drugs, cholesterol-lowering agents, anti-cancer drugs, hypertension drugs, and the like.

### 4. Therapeutic Methods

a. Treating Inflammation

In view of the discovery that IKKε plays a central role in integrating signals induced by pro-inflammatory stimuli (Kravchenko et al.; J. Biol. Chem., 278:26612-26619, 2003); and that IKKε, along with TBK1, has been shown to be involved in maintaining macrophages in an activated inflammatory state following activation of the interferon response (Solis, et al.; Eur. J. Immunol.; 37:529-539, 2007); it is believed that inhibition of IKKε kinase activity, TBK1 kinase activity, or the kinase activities of both IKKε and TBK1 would be effective in treating inflammation resulting from a wide range of causes, including both systemic and chronic inflammation. Hence, the present invention provides methods of treating inflammation, and complications associated with inflammation, comprising administering a therapeutically effective amount of one or more IKKε and/or TBK1-inhibiting compounds according to Formula I to a patient in need of such treatment.

b. Treating Rheumatoid Arthritis (RA)

In view of the discovery that IKKε, as part of a complex kinases, has been found to play a role in the synovial inflammation, extracellular matrix destruction and activation of the anti-viral program and innate immune response in RA (Sweeney et al.; J. Immunol., 174:6424-6430, 2005), it is believed that inhibition of IKKε and/or TBK1 kinase activity would be effective in treating RA. Consequently, the present invention provides methods of treating RA, and complications associated with RA, comprising administering a therapeutically effective amount of one or more IKKε and/or TBK1-inhibiting compounds according to Formula I to a patient in need of such treatment.

c. Treating Systemic Lupus Erythematosus (SLE)

In view of the role of phosphorylated transcription factors IRF3 and IRF7 in mediating the upregulation of IFNα/β and associated type I interferon signature genes that is a hallmark of flare-ups of SLE symptoms in SLE patients, and further view of the roles of IKKε and TBK in respectively phosphorylating IFR3 and IRF7, it is believed that inhibition of IKKε and/or TBK activity might be provide an effective means to reduce the intensity and longevity of such flare-ups in patients suffering from SLE. Consequently, the present invention provides methods of treating SLE, and complications associated with SLE flare-ups, comprising administering a therapeutically effective amount of one or more IKKε and/or TBK1-inhibiting compounds according to Formula I to a patient in need of such treatment.

d. Treating Diseases Associated with Aberrant Accumulation of Cytosolic Nucleic Acids: Sjögrens Syndrome, Aicardi-Goutieres Syndrome, Certain Forms of Systemic Lupus Erythematosus, Chilblain Lupus, Retinal Vasculopathy and Cerebral Leukodystrophy (RVCL)

Sjögrens syndrome, Aicardi-Goutieres syndrome, certain forms of systemic lupus erythematosus, chilblain lupus, RVCL are commonly associated with mutations in at least one of the following genes: TREX1; RNASEH2B; RNASEH2C; RNASEH2A; and SAMHD1 (Crow and Rehwinkel; Aicardi-Goutières syndrome and related phenotypes: linking nucleic acid metabolism with autoimmunity; Hum. Mol. Genet., 18:130-136, 2009; Kavanagh, et al.; New roles for the major human 3′-5′ exonuclease TREX1 in human disease; Cell Cycle, 7:1718-1725, 2008). These proteins are involved in degrading nucleic acids that are aberrantly located in the cytosolic compartment. If nucleic acids accumulate in the cytosol and are recognized by DNA or RNA receptors (i.e., RIG-I, MDA5, DAI, and others) this recognition leads to type I interferon production and autoimmune disease. The TBK1 and IKKε kinases are part of the signal cascade that leads to type I interferon production through phosphorylation of IRF3 and/or IRF7, and NFκB transcription factors (Hornung and Latz; Intracellular DNA Recognition; Nat. Rev. Immunol., 10:123-130, 2010). As such, small molecule inhibitors of IKKε and/or TBK1 kinases are expected to block type I interferon expression and provide therapeutic benefits to patients who are unable to properly degrade aberrantly localized cytosolic nucleic acids. Consequently, the present invention provides methods of treating diseases associated with the abberent accumulation of cytosolic nucleic acids, including Sjögrens syndrome, Aicardi-Goutieres syndrome, certain forms of systemic lupus erythematosus, chilblain lupus, RVCL, and complications associated with these diseases, comprising administering a therapeutically effective amount of one or more IKKε and/or TBK1-inhibiting compounds according to Formula I to a patient in need of such treatment.

e. Treating Systemic Sclerosis

Systemic sclerosis is an autoimmune disease that targets connective tissue. The immune abnormalities cause increased production of extracellular matrix proteins in skin and vascular tissues through the interactions of several cell types, including endothelial cells, lymphocytes, macrophages, and fibroblast cells. A recognized feature of this disease is an abnormal type I interferon-gene expression signature (Assassi, et al.; Systemic sclerosis and lupus: points in an interferon-mediated continuum; Arthritis Rheum., 62:589-598, 2010). As with other autoimmune diseases, the exact cause of systemic sclerosis is not completely understood, but inhibition of type I interferons and fibrogenic cytokines (e.g. TGF-β) through TLR3 pathway inhibition may be therapeutically useful (Farina, et al.; Poly(I:C) Drives Type I IFN- and TGFbeta-Mediated Inflammation and Dermal Fibrosis Simulating Altered Gene Expression in Systemic Sclerosis; J. Invest. Dermato., epub, Jul. 8, 2010). The IKKε and/or TBK1 kinases are essential for production of type I interferon and for TGF-β signaling through TLR3 receptor activation. Small molecule inhibitors of the IKKε & TBK1 kinases, such as the compounds according to Formula I, may benefit patients suffering from systemic sclerosis. Consequently, the present invention provides methods of treating systemic sclerosis, and complications associated with systemic sclerosis, comprising administering a therapeutically effective amount of one or more IKKε and/or TBK1-inhibiting compounds according to Formula I to a patient in need of such treatment.

f. Treating Dermatomyositis and Polymyositis—Subtypes of Myositis

Myositis describes a collection of several poorly defined autoimmune diseases represented by the most common subtypes; dermatomyositis, polymyocitis, and inclusion-body myositis. Production of autoantibodies that target unknown muscle tissue antigens result in muscle weakness and skin abnormalities (Dalakas; Immunotherapy of Myositis: Issues, Concerns and Future Prospects; Nat. Rev. Rheum., 6:129-137, 2010). A recently identified feature of dermatomyositis and polymyositis is an aberrent type I interferon-gene expression signature profile in both muscle and PBMC samples from diseased patients (Baechler, et al.; An Interferon Signature in the Peripheral Blood of Dermatomyositis Patients is Associated with Disease Activity; Mol. Med., 13:59-68, 2007). The interferon-gene signature results from elevated IFN-α/β cytokines that are aberrantly produced. The IKK/TBK1 pathway is essential for the production of IFN-α/β proteins upon activation of TLR3, TLR4, and cytosolic nucleic acid receptors; RIG-I, MDA5, DAI, and others. It is expected that patients suffering from dermatomyositis and polymyocitis would benefit from treatment with small molecule IKKε and/or TBK1 inhibitors such as the compounds according to Formula I. Consequently, the present invention provides methods of treating dermatomyositis and polymyocitis, and complications associated with these diseases, comprising administering a therapeutically effective amount of one or more IKKε and/or TBK1-inhibiting compounds according to Formula I to a patient in need of such treatment.

g. Treating Psoriasis

In view of the fact that psoriasis is a chronic inflammatory skin disorder involving up-regulation of interleukins IL-23, IL-17A and IL-22, and in further view of the discovery that IKKε plays a role in integrating signals induced by pro-inflammatory stimuli (Kravchenko et al.; J. Biol. Chem.; 278:26612-26619, 2003); and that IKKε, along with TBK1, has been shown to play a role in maintaining macrophages in an activated, inflammatory state, following activation of the interferon response (Solis, et al.; Eur. J. Immunol.; 37:529-539, 2007); it is believed that inhibition of IKKε and TBK activity might provide an effective means to treating psoriasis. Consequently, the present invention provides methods of treating psoriasis, and complications associated with psoriasis, comprising administering a therapeutically effective amount of one or more IKKε and/or TBK1-inhibiting compounds according to Formula I to a patient in need of such treatment.

h. Treating Chronic Obstructive Pulmonary Disease (COPD)

COPD is characterized by chronic inflammation of the lungs and narrowing of the airways often caused by cigarette smoke (Churg, et al.; Mechanisms of cigarette smoke-induced COPD: Insights from animal models; Am. J. Physiol. Lung Cell. Mol. Physiol., 294:612-631, 2008). Viral and bacterial infections exacerbate the chronic inflammation in patients with COPD and result in approximately 120,000 deaths each year. Pulmonary infections can be recognized by nucleic acid receptors that activate IKK/TBK1 signaling, leading to proinflammatory chemokine secretion of RANTES, IP-10 and IL-8. These chemokines recruit a variety of proinflammatory cells, including T-cells, eosinophils, basophils, neutrophils, natural killer and dendritic cells, to lungs. Recruitment of proinflammatory cells to the lungs results in lung tissue damage. Eosinophils and T cells play a primary role in causing tissue damage due to their release of cytotoxic proteins and proteases. Inhibition of the IKK/TBK1 pathway is likely to have therapeutic benefits in Asthma and COPD patients. Consequently, the present invention provides methods of treating COPD, and complications associated with COPD, comprising administering a therapeutically effective amount of one or more IKKε and/or TBK1-inhibiting compounds according to Formula I to a patient in need of such treatment.

i. Treating Inflammatory Bowel Disease (IBD)

IBD is an autoimmune-like disorder characterized by chronic inflammation of the intestinal mucosal tissue. The gut is an immunologically unique organ, which must protect the host from pathogens while being tolerant to dietary antigens and essential commensal bacteria. The intestinal wall is therefore an actively regulated barrier. IBD is characterized by a dysregulated immune response to commensal bacteria in genetically susceptible patients. Toll-like receptor (TLR) transmembrane proteins are a central component of the intestinal bacterial surveillance system expressed by intestinal epithelial cells, T cells, antigen-presenting macrophages, and dendritic cells. TLRs have been genetically implicated in IBD based on the identification of single-nucleotide polymorphisms in a number of TLRs (TLR1, 2, 4, 6, and 9) that are associated with increase disease susceptibility or extent of disease in IBD patients (Cario; Toll-like Receptors in Inflammatory Bowel Diseases: A Decade Later; Inflamm. Bowel Dis., 16:1583-1597, 2010). TLR4 is upregulated in IBD, whereas in normal intraepithelial cells it is expressed at such low levels as to be undetectable. TLR4 is a bacterial lipopolysaccharide-recognizing receptor, and one of the outputs from the TLR4 receptor signaling complex involves IKKε and/or TBK1 kinases. This pathway directs the activation of the transcription factor IRF3 via phosphorylation by IKKε and/or TBK1 kinase, which induces expression of proinflammatory chemokines RANTES and MCP 1. Modulation of overactive TLR4 signaling, via inhibition of the IKK/TBK1 signaling pathway by a compound of the present invention may have therapeutic benefit to IBD patients. Consequently, the present invention provides methods of treating IBD, and complications associated with IBD, comprising administering a therapeutically effective amount of one or more IKKε and/or TBK1-inhibiting compounds according to Formula I to a patient in need of such treatment.

j. Treating Obesity, Insulin Resistance, Type 2 Diabetes (NIDDM), and Metabolic Syndrome

In view of the discovery that IKKε knockout mice were protected from high-fat diet-induced obesity, chronic inflammation in liver and fat, hepatic steatosis, and whole-body insulin resistance; and in further view of the fact that these IKKε knockout mice were found to have increased energy expenditure and thermogenesis, maintained insulin sensitivity in both liver and fat, reduced expression of inflammatory cytokines, and altered expression of regulatory proteins and enzymes involved in glucose and lipid metabolism (Chiang et al.; Cell, 138:961-975, 2009); it is believed that inhibition of IKKε kinase activity would be effective in treating obesity, insulin resistance, NIDDM, and metabolic syndrome, and complications associated with these and other metabolic diseases and disorders. Consequently, the present invention provides methods of treating obesity, insulin resistance, metabolic syndrome, type 2 diabetes, and complications associated with these diseases, and other metabolic diseases and disorders, comprising administering a therapeutically effective amount of one or more IKKε and/or TBK1-inhibiting compounds according to Formula I to a patient in need of such treatment.

In further view of the discovery that TBK1 mediates phosphorylation of insulin receptor at serine residue 994, and thereby provides a potential link between inflammation and insulin resistance (Muñoz et al; J. Endocrinol., 201:185-197, 2009), it is believed that inhibition of TBK1 kinase activity might be effective in treating insulin resistance. Consequently, the present invention provides methods of treating insulin resistance, and complications associated with insulin resistance, comprising administering a therapeutically effective amount of one or more IKKε and/or TBK1-inhibiting compounds according to Formula I to a patient in need of such treatment.

k. Treating Cancer:

In view of the discovery that the gene encoding IKKε (i.e., IKBKE; Entrez Gene Gene ID: 9641) has been identified as a breast cancer oncogene (Boehm, et al.; Cell; 129:1065-1079, 2007); that IKKε directly phosphorylates the tumor suppressor CYLD in vivo, thereby decreasing the activity of CYLD, and leading to transformation and turmorigenesis (Hutti, et al.; Mol. Cell; 34:461-472, 2009); and that overexpression of IKKε is a recurrent event in human ovarian cancer, and that this overexpression could play a pivotal role in both tumor progression and the development of cisplatin resistance (Guo, et al.; Am. J. Pathol.; 175:324-333, 2009); it is believed that inhibition of IKKε kinase activity would be effective in treating of a wide range of cancers. Consequently, the present invention provides methods of treating a wide range of cancers comprising administering a therapeutically effective amount of one or more IKKε-inhibiting compounds according to Formula I to a patient in need of such treatment.

In further view of the discovery that GTPase-mediated activation of TBK1 couples innate immune signaling to tumor cell survival (Chien et al.; Cell; 127:157-170, 2006), it is believed that inhibition of TBK1 kinase activity would be effective in treating of a wide range of cancers. Consequently, the present invention provides methods of treating a wide range of cancers comprising administering a therapeutically effective amount of one or more TBK1-inhibiting compounds according to Formula I to a patient in need of such treatment.

As used herein, the term “cancer” has its conventional meaning in the art. Cancer includes any condition of the animal or human body characterized by abnormal cellular proliferation. The cancers to be treated comprise a group of diseases characterized by the uncontrolled growth and spread of abnormal cells. Compounds of the invention have been shown to be effective in cell-based cancer models, and are thus thought to have utility in treating a broad range of cancers. However, therapeutic methods of the present invention would best be directed towards cancers that are found to respond favorably to treatment with an IKKε and/or TBK1 kinase inhibitor. Further, “treating cancer” should be understood as encompassing treating a patient who is at any one of the several stages of cancer, including diagnosed but as yet asymptomatic cancer. A patient having cancer can be identified by conventional diagnostic techniques known in the art, and the identified patient may be treated with a compound of the present invention, once their cancer has been found to be susceptible to treatment with an IKKε and/or TBK1 kinase inhibitor.

As noted, cancers that may be treated by the methods of the invention are those cancers that respond favorably to treatment with an IKKε and/or TBK1 kinase inhibitor. Such cancers may include, but are not limited to, Hodgkin's disease, non-Hodgkin's lymphoma, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, and prostatic carcinoma.

The present invention further provides methods for combination therapy for treating cancer by treating a patient (either a human or another animal) in need of such treatment with a compound of the present invention together with one or more other anti-cancer therapies. Such other anti-cancer therapies include traditional chemotherapy agents, targeted agents, radiation therapy, surgery, hormone therapy, etc. In the combination therapy, the compound of the present invention may be administered separately from, or together with the one or more other anti-cancer therapies.

As noted above, it is believed that inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer are disease and disorders that will respond favorably to therapy with an IKKε or TBK1 kinase inhibitor. Consequently, the present invention provides therapeutic methods for treating inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders. These therapeutic methods involve treating a patient (either a human or another animal) in need of such treatment, with a therapeutically effective amount of at least one compound according to Formula I, or a pharmaceutical composition comprising a therapeutically effective amount of at least one compound according to Formula I. These therapeutic methods also administering to a patient (either a human or another animal) in need of such treatment, a therapeutically effective amount of at least one compound according to Formula I, or a pharmaceutical composition comprising a therapeutically effective amount of at least one compound according to Formula I.

It is believed that compounds according to Formula I and having an IKKε kinase inhibitory activity (IC50 value) of less than about 0.005 μM (5 nM), as determined in the in-vitro IKKε kinase inhibition assays as described below, are sufficiently active for the therapeutic methods proposed. These compounds include, for example, Example Compounds 2, 3, 4, 5, 6, 11, 14, 15, 16, 18, 20, 21, 22, 24, 25, 26, 27, 28, 29, 30, 31, 32, 33, 34, 35, 59, 68, 72, 73, 75, 76, 80, 82, 83, 88, 91, 93, 96, 98, 100, 103, 104, 107, 111, 114, 115, 118, 124, 127, 129, 130, 132, 134, 155, 157, 158, 164, 165, 171, 176, 178, 181, 184, 190, 191, 206, 208, 210, 211, 212, 216, 223, 225, 231, 235, 237, 239, 242, 246, 253, 256, 261, 262, 264, 271, 275, 287, 290, 307, 311, 326, 329, 331, 334, 335, 341, 354, 367, 370, 371, 373, 374, 376, 377, 381, 385, 392, 393, 394, 395, 396, 397, 400, 401, 402, 403, 404, 405, 406, 413, 415, 436, 437, 438, 439, 440, 442, 444, 446, 467, 471, 475, 476, 477, 478, 479, 480, 481, 482, 484, 485, 486, 487, 488, 489, 490, 492, 493, 494, 495, 496, 497, 498, 500, 501, 502, 503, 504, 505, 506, 507, 510, 511, 512, 517, 518, 519, 520, 521, 522, 523, 524, 525, 526, 527, 529, 530, 531, 533, 534, 535, 536, 538, 539, 540, 541, 542, 543, 544, 545, 546, 547, 548, 549, 550, 552, 558, 559, 560, 561, 563, 564, 565, 566, 567, 571, 572, 573, 574, 575, 576, 577, 578, 579, 580, 581, 583, 584, 585, 586, 587, 588, 589, 590, 591, 592, 593, 594, 595, 596, 597, 598, 599, 601, 603, 604, 606, 607, 608, 609, 610, 611, 612, 613, 614, 615, 616, 617, 618, 619, 620, 621, 622, 624, 625, 626, 627, 628, 629, 630, 631, 632, 635, 636, 637, 638, 639, 640, 642, 643, 644, 645, 646, 647, 648, 649, 650, 651, 653, 654, 655, 656, 657, 658, 659, 661, 662, 664, 665, 666, 667, 668, 669, and 670, as identified below.

The present invention also comprises treating isolated cells with a therapeutically effective amount of at least one compound according to Formula I, or a pharmaceutical composition comprising a therapeutically effective amount of at least one compound according to Formula I.

As used herein, the phrase “treating . . . with . . . a compound” means either administering a compound according to Formula I, or a pharmaceutical compositions comprising a compound according to Formula I, directly to isolated cells or to an animal, or administering to cells or an animal another agent to cause the presence or formation of a compound according to Formula I inside the cells or the animal. Consequently, the methods of the present invention comprise administering to cells in vitro or to a warm-blood animal, particularly a mammal, and more particularly a human, a pharmaceutical composition comprising an effective amount of at least one compound according to Formula I, or causing the presence or formation of at least one compound according Formula I inside the cells or the animal.

As would be appreciated by the skilled artisan, at least one therapeutic compound according to Formula I may be administered in one dose at one time, or may be divided into a number of smaller doses to be administered at predetermined intervals of time. The suitable dosage unit for each administration may be determined based on the effective daily amount and the pharmacokinetics of the compounds. In the case of combination therapy, a therapeutically effective amount of one or more other therapeutically effective compound can be administered in a separate pharmaceutical composition, or alternatively included in the pharmaceutical composition according to the present invention which contains a compound according to the present invention. The pharmacology and toxicology of many therapeutically effective compounds are known in the art. See e.g., Physicians Desk Reference, Medical Economics, Montvale, N.J.; and The Merck Index, Merck & Co., Rahway, N.J. The therapeutically effective amounts and suitable unit dosage ranges of such compounds used in art can be equally applicable in the present invention.

It should be understood that the dosage range set forth herein is exemplary and is not intended to limit the scope of the present invention. The therapeutically effective amount for each active compound of the invention may vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration may be adjusted as the various factors change over time.

The present invention also provides methods for methods for combination therapy for treating inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders, by treating a patient in need thereof, with a therapeutically effective amount of at least one compound according to Formula I, together with a therapeutically effective amount of one or more other compounds that have been shown to be effective in the treatment of inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders.

For the convenience of combination therapy, at least one compound according to Formula I can be administered together in the same formulation with the one or more other compounds that have been shown to be effective in the treatment of inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders, in the same formulation or dosage form. Thus, the present invention also provides pharmaceutical compositions or medicaments for combination therapy, comprising an effective amount of at least one compound according to Formula I, and an effective amount of at least one other compound that has been shown to be effective in the treatment of inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders.

### 5. Methods of Making the Compounds According to Formula I

Methods of making the compounds according to Formula I, and intermediates used in their synthesis, are provided in the Examples section below. Apprised of the general synthetic schemes, specific intermediates, and detailed example of specific syntheses disclosed in the following section, the skilled artisan would be readily enabled to make the remaining compounds disclosed in Table 2. In all cases, the syntheses were begun using commercially-available starting materials.

### EXAMPLES

### Chemical Examples

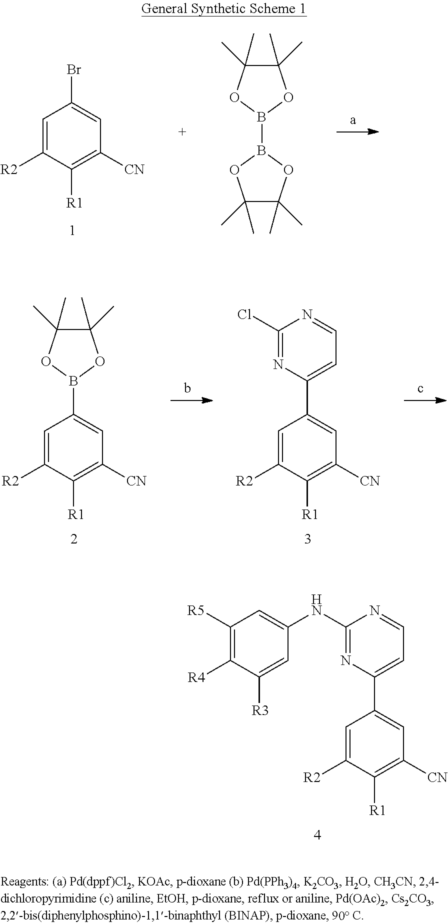

Generally speaking, the compounds according to Formula I can be synthesized using methods known in the art combined with the disclosure herein. In general, compounds according to Formula I can be synthesized according to Scheme 1. For example, 3-bromo benzonitriles, 1, were converted to the corresponding boranyl benzonitriles 2 by treatment with dichloro-(1,2-bis-(diphenylphosphino)ethane)-palladium(II) (Pd(dppf)Cl₂)) and bis(pinacolato)diboron in the presence of KOAc in p-dioxane. Conversion to the chloro pyrimidines 3 was achieved by reacting the boranyl esters with dichloropyrimidine in the presence of Pd(PPh₃)₄. Reaction with anilines under thermal conditions in EtOH and p-dioxane or under catalytic conditions with Pd(OAc)₂, BINAP and cesium carbonate in p-dioxane gives the aryl pyrimidines 4.

### Preparation of Intermediates

### Standard Methods

### Standard Method a; Nitro Reduction

The nitro compound was hydrogenated for 4-18 hours (h) in MeOH with catalytic Pd/C. The suspension was filtered through Celite® (World Minerals, Inc.; Santa Barbara, Calif.) and concentrated to provide the aniline. If required, purification was performed by MPLC (SiO₂, EtOAc/Hexanes, 0-100%, optionally followed by a gradient from 100% EtOAc to 100% of 1:1 CH₂Cl₂/MeOH).

### Standard Method B; Phenol Alkylation

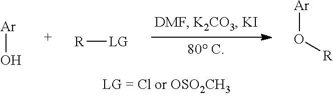

A solution of the nitrophenol, chloro or mesylated compound, K₂CO₃ (1.1 equivalents (eq)) and KI (catalytic) in DMF was heated to 80° C. overnight (o/n). The reaction was diluted with EtOAc, washed with brine, dried (MgSO₄), filtered and concentrated. Purification by MPLC (SiO₂, EtOAc/Hexanes, 0-100%, optionally followed by a gradient from 100% EtOAc to 100% of 1:1 CH₂Cl₂/MeOH) provided the desired compounds.

### Standard Method C; O-Mesylation

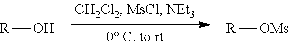

A solution of the alkyl alcohol and Et₃N (1.1 to 5 eq) in CH₂Cl₂ was treated with methanesulfonyl chloride (1.1 eq) at 0° C. and allowed to warm to room temperature (rt) and stirred for 1 to 18 hours (h). The reaction was diluted with CH₂Cl₂, washed with 5% NaOH or H₂O and brine, dried (MgSO₄), filtered, and concentrated to provide the desired compounds.

### Standard Method D; N Protection as BOC

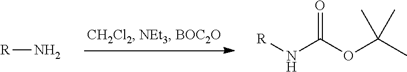

A solution of the amine and Et₃N (1.1 eq) in CH₂Cl₂ was treated with BOC₂O (1.1 eq) and allowed to stir o/n. The reaction was washed with brine, dried (MgSO₄), filtered, and concentrated to provide the desired compounds.

### Standard Method E; BOC Deprotection

A solution of the BOC protected amine in tetrahydrofuran (THF) was treated with trifluoroacetic acid (TFA) (1%) o/n. The reaction was concentrated onto Celite® and purified by RP-MPLC (C₁₈, MeOH/H₂O, 0-100% with (w/) 0.1% TFA) to provide the desired compounds as the TFA salts.

### Standard Method F; CDI Coupling

A solution of the aniline in THF was treated with CDI (2.1 eq) for 1-18 h. The amine was added (excess) and the reaction stirred for 2-18 h. The reaction was concentrated onto Celite® and purified by RP-MPLC (C₁₈, MeOH/H₂O, 0-100% w/0.1% TFA) to provide the desired compounds.

### Standard Method G; Ester Hydrolysis

A solution of the ester in THF/H₂O (2:1) was treated with LiOH (1.0-10 eq) at 25-65 C for 1-18 h. A 1N solution of aqueous (aq.) HCl was added until pH 4-5. The precipitate was collected, washed with H₂O and dried under high vacuum to provide the desired compound.

### Standard Method H; HATU Coupling

A solution of the carboxylic acid, the amine (1.0-1.5 eq), N,N-diisopropylethylamine (DIPEA) (1.0-1.5 eq) in an appropriate solvent, was added 2-(7-Aza-1H-benzotriazole-1-yl)-1,1,3,3-tetramethyluronium hexafluorophosphate (HATU) (1.0-1.5 eq). The reaction mixture was stirred at rt for 16 h. The solvent was evaporated and the residue purified by RP-MPLC (C¹⁸, MeOH/H₂O, 0-100% w/0.1% TFA) to provide the desired compounds. The desired fractions were collected and the solvent evaporated under reduced pressure. The resulting solid was recrystallized from EtOAc/Hexanes to afford the desired compound.

### Specific Syntheses

### Preparation of Intermediate I-1; 2-Amino-5-(2-{[4-(morpholin-4-yl)phenyl]amino}pyrimidin-4-yl)benzonitrile

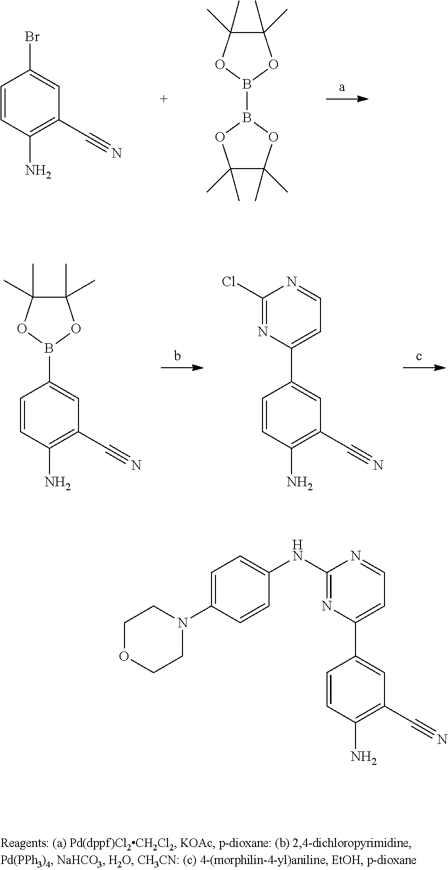

Step 1. 2-Amino-5-(4,4,5,5-tetramethyl-1,3,2-dioxaborolan-2-yl)benzonitrile: To a solution of 2-amino-5-bromobenzonitrile (1.0 g, 5.075 mmol) in p-dioxane (15 mL), bis(pinacolato)diborane (1.95 g, 7.61 mmol), KOAc (1.5 g, 15.23 mmol), and Pd(dppf)Cl₂CH₂Cl₂ (0.207 g, 0.25 mmol) were added. The resulting mixture was stirred for 16 h at 80° C. The cooled reaction crude was diluted with 200 mL EtOAc, washed with H₂O and brine, dried (Na₂SO₄), filtered, and concentrated in vacuo. The residue was purified by column chromatography on SiO₂ (Hexanes/EtOAc) to afford the title compound (1.13 g, 91%).

Step 2. 2-Amino-5-(2-chloropyrimidin-4-yl)benzonitrile: To a solution of 2-amino-5-(4,4,5,5-tetramethyl-1,3,2-dioxaborolan-2-yl)benzonitrile (1.1 g, 4.5 mmol) in CH₃CN (30 mL) and H₂O (10 mL), 2,4-dichloropyrimidine (0.672 g, 4.5 mmol), NaHCO₃ (1.14 g, 13.5 mmol), and Pd(PPh₃)₄ (0.26 g, 0.225 mmol) were added. The resulting mixture was stirred for 5 h at 80° C.

Upon cooling, the desired product precipitates from solution, was washed with 3:1 CH₃CN/H₂O mixture and dried in vacuo to afford the title compound (0.67 g, 65%).

Step 3. 2-Amino-5-(2-{[4-(morpholin-4-yl)phenyl]amino}pyrimidin-4-yl)benzonitrile: To a solution of 2-amino-5-(2-chloropyrimidin-4-yl)benzonitrile (0.231 g, 1 mmol) in EtOH (15 mL) and p-dioxane (15 mL), 4-(morphilin-4-yl)aniline (0.267 g, 1.5 mmol) was added. The resulting mixture was stirred for 3 days (d) at 100° C. Upon cooling, the resulting precipitate was triturated with warm MeOH/EtOAc (1:4 mixture) and dried in vacuo to afford the title compound (0.3 g, 80%). ¹H NMR (DMSO-d₆) δ 9.33 (s, 1H), 8.38 (d, 1H), 8.25 (m, 1H), 8.12 (dd, 1H) 7.64 (d, 2H) 7.23 (d, 1H), 6.88-6.96 (m, 3H), 6.67 (s, 2H), 3.74 (m, 4H), 3.04 (m, 4H). LC-MS [M+H]⁺ 373.1.

### Preparation of Intermediate I-2; 5-(2-Chloropyrimidin-4-yl)-2-methoxybenzonitrile

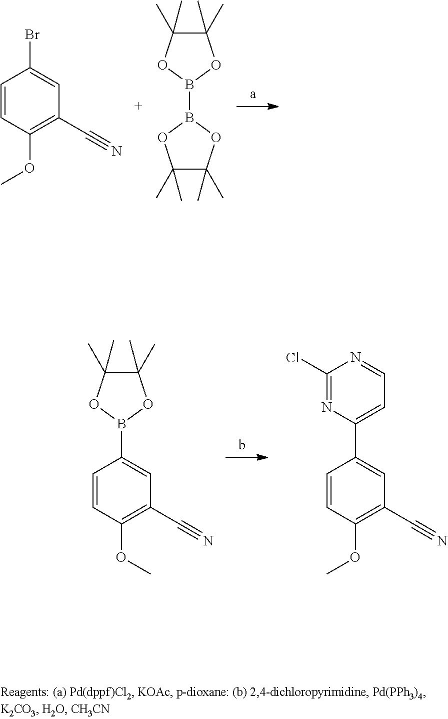

Step 1. 2-Methoxy-5-(4,4,5,5-tetramethyl-1,3,2-dioxaborolan-2-yl)benzonitrile: To a solution of 2-methoxy-5-bromobenzonitrile (5.0 g, 23.6 mmol) in p-dioxane (125 mL), bis(pinacolato)diborane (9.0 g, 35.4 mmol), KOAc (7.0 g, 71.3 mmol), and Pd(dppf)Cl₂ (0.863 g, 1.17 mmol) were added. The resulting mixture was stirred for 18 h at 80° C. The cooled reaction crude was diluted with 1200 mL EtOAc, washed with H₂O and brine, dried (Na₂SO₄), filtered, and concentrated in vacuo. The residue was purified by column chromatography on SiO₂ (Hexanes/EtOAc) to afford the title compound (5.6 g, 92%).

Step 2. 5-(2-Chloropyrimidin-4-yl)-2-methoxybenzonitrile: To a solution of 2-methoxy-5-(4,4,5,5-tetramethyl-1,3,2-dioxaborolan-2-yl)benzonitrile (5.6 g, 21.6 mmol) in CH₃CN (100 mL) and H₂O (35 mL), 2,4-dichloropyrimidine (3.22 g, 21.6 mmol), K₂CO₃ (9.0 g, 65 mmol), and Pd(PPh₃)₄ (1.25 g, 1.06 mmol) were added. The resulting mixture was stirred for 5 h at 90° C. Upon cooling, the product precipitated from solution and was filtered and washed with a 3:1 CH₃CN/H₂O mixture, and dried in vacuo to afford the title compound (4.04 g, 76%). ¹H NMR (CDCl₃) δ 8.66 (d, 1H), 8.36-8.33 (m, 2H), 7.59 (d, 1H), 7.13-7.11 (m, 1H), 4.04 (s, 3H). LC-MS [M+H]⁺ 245.9.

### Preparation of Intermediate I-3; 2-Hydroxy-5-[2-(4-morpholin-4-yl-phenylamino)-pyrimidin-4-yl]-benzonitrile

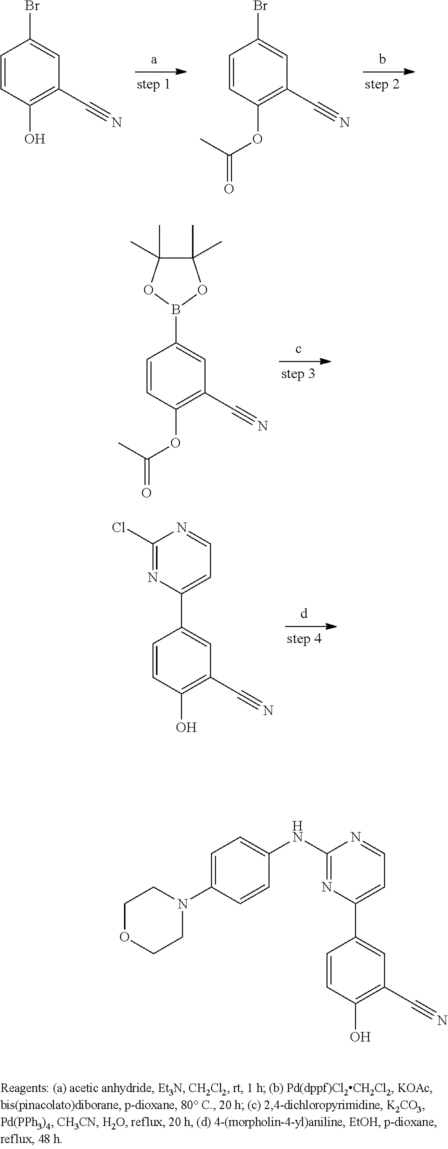

Step 1. 4-Bromo-2-cyanophenyl acetate: To a solution of 5-bromo-2-hydroxy-benzonitrile (3.96 g, 20.0 mmol) and Et₃N (6 mL) in CH₂Cl₂ (60 mL) was added Ac₂O (4 mL, 42.4 mmol) at rt. After stirring for 1 h at rt, the mixture was diluted with CH₂Cl₂ (100 mL), washed with H₂O (100 mL) and brine (100 mL), dried (MgSO₄) and concentrated in vacuo. The residue (4.7 g, 19.6 mmol) was used without further purification.

Step 2. 2-Cyano-4-(4,4,5,5-tetramethyl-1,3,2-dioxaborolan-2-yl)phenyl acetate: To a solution of 4-bromo-2-cyanophenyl acetate (4.7 g, 19.6 mmol) in p-dioxane (100 mL) was added Pd(dppf)Cl₂.CH₂Cl₂ (0.80 g, 0.98 mmol), and KOAc (5.86 g, 60 mmol). After stirring at 80° C. for 20 h, the mixture was filtered to remove salts, and the filtrate was concentrated under reduced pressure. The residue was purified by column chromatography (SiO₂, EtOAc/Hexanes, 0-50%) to afford the title compound (4.2 g, 75%).

Step 3. 5-(2-Chloropyrimidin-4-yl)-2-hydroxybenzonitrile: To a solution of 2-cyano-4-(4,4,5,5-tetramethyl-1,3,2-dioxaborolan-2-yl)phenyl acetate (4.2 g, 14.6 mmol) in CH₃CN (100 mL) and H₂O (40 mL) was added K₂CO₃ (6.04 g, 43.8 mmol) and Pd(PPh₃)₄ (0.84 g, 0.73 mmol). After refluxing for 20 h, the mixture was concentrated to remove CH₃CN, and the product was extracted with a solution of i-PrOH/CHCl₃ (1:3) (200 mL). The organic solution was washed with brine (100 mL), dried (MgSO₄) and concentrated under reduced pressure. The residue was purified by column chromatography (SiO₂, MeOH 020% in CH₂Cl₂ with 0.1% NH₄OH) to give the title compound (3.0 g, 88%); LC-MS [M-1]229.

Step 4. 2-Hydroxy-5-(2-{[4-(morpholin-4-yl)phenyl]amino}pyrimidin-4-yl)benzonitrile. A solution of 5-(2-chloropyrimidin-4-yl)-2-hydroxybenzonitrile (0.89 g, 3.84 mmol) and 4-(morpholin-4-yl)aniline (1.03 g, 5.77 mmol) in EtOH (10 mL) and p-dioxane (10 mL) was stirred at reflux for 48 h. After concentrating under reduce pressure, the residue was purified by reverse phase column chromatography (C₁₈, CH₃CN 95% in H₂O with 0.1% TFA) to give the title compound (0.80 g, 56%). ¹H NMR (DMSO-d₆) δ 9.43 (s, 1H), 8.45 (d, 1H), 8.42 (d, 1H), 8.32-8.29 (m, 1H), 7.65-7.62 (m, 2H), 7.32 (d, 1H), 7.15 (d, 1H), 6.94-6.91 (m, 2H), 3.76-3.73 (m, 4H), 3.06-3.03 (m, 4H). TOF LC-MS [M+H]⁺ 374.1662.

### Preparation of Intermediate I-4; 5-(2-Chloropyrimidin-4-yl)-2-(tetrahydro-2H-pyran-4-yloxy)benzonitrile

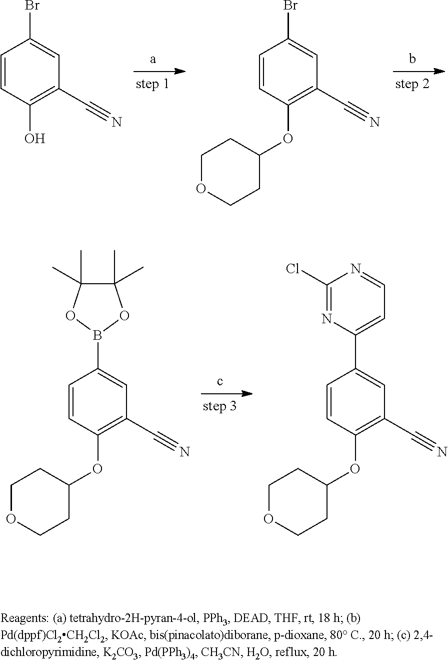 [SMILES: N#Cc1cc(Br)ccc1O ; N#Cc1cc(Br)ccc1OC1CCOCC1 ; CC1(C)OB(c2ccc(OC3CCOCC3)c(C#N)c2)OC1(C)C ; N#Cc1cc(-c2ccnc(Cl)n2)ccc1OC1CCOCC1]

Step 1. 5-Bromo-2-(tetrahydro-2H-pyran-4-yloxy)benzonitrile: To a solution of 5-bromo-2-hydroxy-benzonitrile (1.98 g, 10.0 mmol) in dry THF (40 mL) was added tetrahydro-2H-pyran-4-ol (1.02 g, 10 mmol), PPh₃ (3.15 g, 12 mmol), followed by addition of DEAD (1.89 mL, 12 mmol) at rt. After stirring at rt for 18 h, the reaction mixture was concentrated under reduced pressure. The residue was purified by column chromatography (SiO₂, EtOAc/Hexanes, 0-80%) to afford the title compound (2.7 g, 96%). ¹H NMR (DMSO-d₆) δ 8.02 (d, 1H), 7.81 (dd, 1H), 7.35 (d, 1H), 4.85-4.78 (m, 1H), 3.86-3.80 (m, 2H), 3.55-3.47 (m, 2H), 2.01-1.96 (m, 2H), 1.67-1.58 (m, 2H).

Step 2. 2-(Tetrahydro-2H-pyran-4-yloxy)-5-(4,4,5,5-tetramethyl-1,3,2-dioxaborolan-2-yl)benzonitrile: To a solution of 5-bromo-2-(tetrahydro-2H-pyran-4-yloxy)benzonitrile (2.7 g, 9.6 mmol) in p-dioxane (50 mL) was added Pd(dppf)Cl₂.CH₂Cl₂ (0.408 g, 0.50 mmol), and KOAc (2.94 g, 30 mmol). After stirring at 80° C. for 20 h, the mixture was filtered to remove KOAc and the filtrate was concentrated under reduced pressure. The residue was purified by column chromatography (SiO₂, EtOAc/Hexanes, 0-60%) to afford the title compound (3.1 g, 98%).

Step 3. 5-(2-Chloropyrimidin-4-yl)-2-(tetrahydro-2H-pyran-4-yloxy)benzonitrile: To a solution of 2-(tetrahydro-2H-pyran-4-yloxy)-5-(4,4,5,5-tetramethyl-1,3,2-dioxaborolan-2-yl)benzonitrile (3.1 g, 9.4 mmol) in CH₃CN (40 mL) and H₂O (15 mL) was added K₂CO₃ (4.14 g, 30 mmol) and Pd(PPh₃)₄ (0.58 g, 0.5 mmol). After refluxing for 20 h, the mixture was concentrated to remove CH₃CN and the residue was extracted with EtOAc (200 mL). The organic solution was washed with brine (100 mL), dried (MgSO₄), and concentrated under reduced pressure. The residue was purified by column chromatography (SiO₂, EtOAc/Hexanes, 0-100%) to give the title compound (1.3 g, 41%). ¹H NMR (DMSO-d₆) δ 8.83 (d, 1H), 8.60 (d, 1H), 8.46 (dd, 1H), 8.21 (d, H), 7.57 (d, 1H), 5.00-4.94 (m, 1H), 3.90-3.84 (m, 2H), 3.58-3.53 (m, 2H), 2.06-1.99 (m, 2H), 1.73-1.65 (m, 2H).

### Preparation of Intermediate I-5; tert-Butyl 4-[2-cyano-4-(2-{[4-(morpholin-4-yl)phenyl]amino}pyrimidin-4-yl)phenoxy]piperidine-1-carboxylate

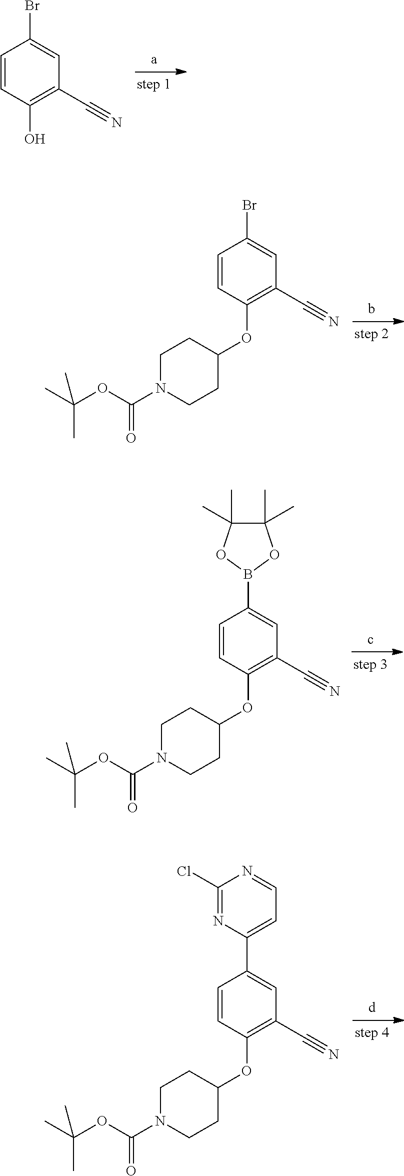 [SMILES: N#Cc1cc(Br)ccc1O ; CC(C)(C)OC(=O)N1CCC(Oc2ccc(Br)cc2C#N)CC1 ; CC(C)(C)OC(=O)N1CCC(Oc2ccc(B3OC(C)(C)C(C)(C)O3)cc2C#N)CC1 ; CC(C)(C)OC(=O)N1CCC(Oc2ccc(-c3ccnc(Cl)n3)cc2C#N)CC1]

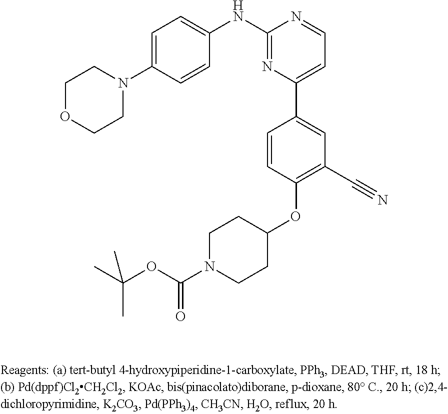 [SMILES: CC(C)(C)OC(=O)N1CCC(Oc2ccc(-c3ccnc(Nc4ccc(N5CCOCC5)cc4)n3)cc2C#N)CC1]

Step 1. tert-Butyl 4-(4-bromo-2-cyanophenoxy)piperidine-1-carboxylate: To a solution of 5-bromo-2-hydroxy-benzonitrile (1.98 g, 10.0 mmol) in dry THF (40 mL) was added tert-butyl 4-hydroxypiperidine-1-carboxylate (2.41 g, 12 mmol), PPh₃ (3.14 g, 12 mmol), followed by addition of DEAD (1.89 mL, 12 mmol) at rt. After stirring at rt for 18 h, the reaction mixture was concentrated under reduced pressure. The residue was purified by column chromatography (SiO₂, EtOAc/Hexanes, 0-80%) to afford the title compound (3.4 g, 89.2%).

Step 2. tert-Butyl 4-[2-cyano-4-(4,4,5,5-tetramethyl-1,3,2-dioxaborolan-2-yl)phenoxy]piperidine-1-carboxylate: To a solution of tert-butyl 4-(4-bromo-2-cyanophenoxy)piperidine-1-carboxylate (3.4 g, 8.92 mmol) in p-dioxane (60 mL) was added Pd(dppf)Cl₂.CH₂Cl₂ (0.364 g, 0.446 mmol), and KOAc (2.65 g, 27 mmol). After stirring at 80° C. for 20 h, the mixture was filtered to remove KOAc, and the filtrate was concentrated under reduced pressure. The residue was purified by column chromatography (SiO₂, EtOAc/Hexanes, 0-100%) to afford the title compound (3.8 g, 99%).

Step 3. tert-Butyl 4-[4-(2-chloropyrimidin-4-yl)-2-cyanophenoxy]piperidine-1-carboxylate: To a solution of tert-butyl 4-[2-cyano-4-(4,4,5,5-tetramethyl-1,3,2-dioxaborolan-2-yl)phenoxy]piperidine-1-carboxylate (3.8 g, 8.90 mmol) in CH₃CN (50 mL) and H₂O (20 mL) was added K₂CO₃ (4.14 g, 30 mmol) and Pd(PPh₃)₄ (0.58 g, 0.5 mmol). After refluxing for 20 h, the mixture was concentrated and the product was extracted with EtOAc (200 mL). The organic solution was washed with brine (100 mL), dried (MgSO₄), and concentrated under reduced pressure. The residue was purified by column chromatography (SiO₂, EtOAc/Hexanes, 0-100%) to give the title compound (2.6 g, 70.5%); LC-MS [M+Na]⁺ 437.

Step 4. tert-Butyl 4-[2-cyano-4-(2-{[4-(morpholin-4-yl)phenyl]amino}pyrimidin-4-yl)phenoxy]piperidine-1-carboxylate: To a solution of tert-Butyl 4-[4-(2-chloropyrimidin-4-yl)-2-cyanophenoxy]piperidine-1-carboxylate (1.25 g, 3.0 mmol) and 4-(morpholin-4-yl)aniline (0.801 g, 4.5 mmol) in EtOH (10 mL) and p-dioxane (10 mL) was stirred at reflux for 48 h. After concentrating under reduce pressure, the residue was purified by column chromatography (SiO₂, EtOAc/Hexanes, 0-100%) to give the title compound (1.5 g, 89.8%); LC-MS (M+1) 587.300.

### Preparation of Intermediate I-6; 5-(2-{[4-(Morpholin-4-yl)phenyl]amino}pyrimidin-4-yl)-2-(piperidin-4-yloxy)benzonitrile

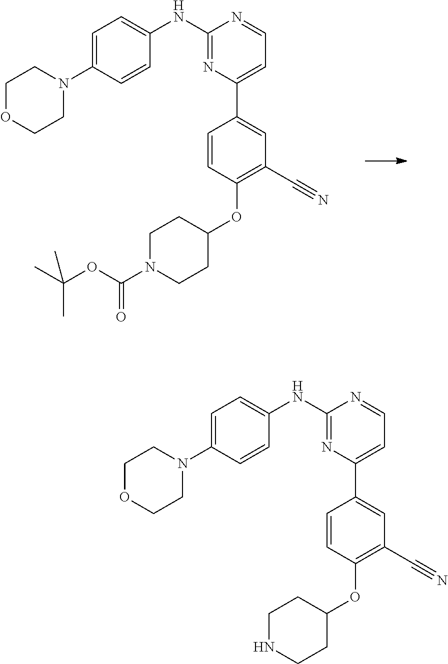 [SMILES: CC(C)(C)OC(=O)N1CCC(Oc2ccc(-c3ccnc(Nc4ccc(N5CCOCC5)cc4)n3)cc2C#N)CC1 ; N#Cc1cc(-c2ccnc(Nc3ccc(N4CCOCC4)cc3)n2)ccc1OC1CCNCC1]

To a solution of tert-butyl 4-[2-cyano-4-(2-{[4-(morpholin-4-yl)phenyl]amino}pyrimidin-4-yl)phenoxy]piperidine-1-carboxylate (1.0 g, 1.79 mmol) in CH₂Cl₂ (20 mL) was added TFA (4 mL) at rt. After stirring at rt for 18 h, the reaction mixture was concentrated under reduced pressure, and the residue was added to H₂O (50 mL) and the mixture basified with K₂CO₃ resulting in the formation of a precipitate which was filtered and dried in vacuo. The crude compound was purified by reverse phase column chromatography (C₁₈, CH₃CN 95% in H₂O with 0.1% TFA) to give the title compound as the corresponding TFA salt. ¹H NMR (DMSO-d₆) δ 8.55-8.45 (m, 3H), 7.68 (d, 1H), 7.56 (d, 1H), 7.43 (d, 1H), 7.21 (d, 1H), 7.05-7.01 (m, 3H), 5.04-4.99 (m, 1H), 3.79-3.73 (m, 6H), 3.25-3.11 (m, 8H), 2.99-2.92 (m, 1H), 2.22-1.90 (m, 4H). TOF LC-MS [M+H]⁺ 456.2134.

### Preparation of Intermediate I-7; tert-Butyl N-[2-[2-(4-amino-2-methoxy-phenoxy)ethoxy]ethyl]carbamate

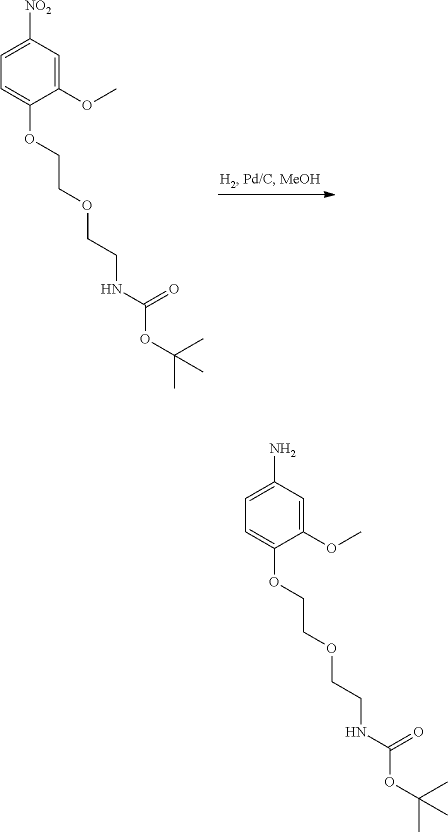 [SMILES: COc1cc([N+](=O)[O-])ccc1OCCOCCNC(=O)OC(C)(C)C ; COc1cc(N)ccc1OCCOCCNC(=O)OC(C)(C)C]

tert-Butyl N-[2-[2-(2-methoxy-4-nitro-phenoxy)ethoxy]ethyl]carbamate (3.32 g, 9.33 mmol) was hydrogenated o/n with 10% Pd/C (catalytic amount) in MeOH. The suspension was filtered, and concentrated to provide the title compound. ¹H NMR (CDCl₃) δ 6.77 (d, 1H), 6.30 (d, 1H), 6.21 (dd, 1H), 5.13 (br s, 1H), 4.10-4.05 (m, 2H), 3.82 (s, 3H), 3.80-3.75 (m, 2H), 3.62-3.56 (m, 2H), 3.38-3.30 (m, 2H), 1.44 (s, 9H).

### Preparation of Intermediate I-8; tert-Butyl N-[2-(2-methoxy-4-nitro-phenoxy)ethyl]carbamate

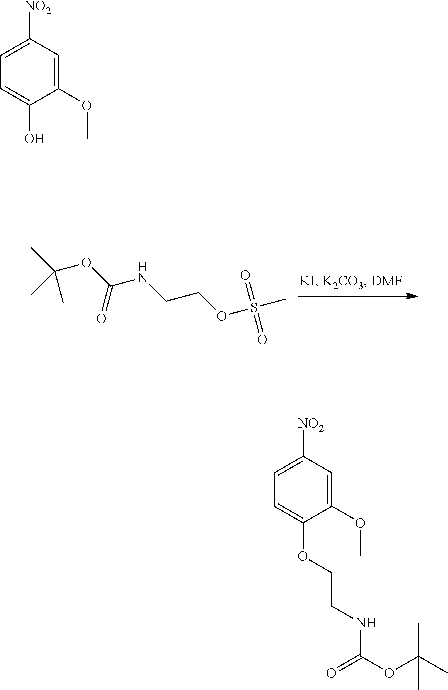 [SMILES: COc1cc([N+](=O)[O-])ccc1O ; CC(C)(C)OC(=O)NCCOS(C)(=O)=O ; COc1cc([N+](=O)[O-])ccc1OCCNC(=O)OC(C)(C)C]

A mixture of 2-methoxy-4-nitro-phenol (194 mg, 1.15 mmol), 2-(tert-butoxycarbonylamino)ethyl methanesulfonate (248 mg, 1.04 mmol), K₂CO₃ (171 mg, 1.23 mmol) and KI (catalytic) in DMF (2 mL) was heated to 80° C. for 4 h. After cooling to rt the reaction was diluted with EtOAc, washed with brine, dried (MgSO₄), filtered, and concentrated. Purification by MPLC (SiO₂, EtOAc/Hexanes, 0-100%) provided the title compound. ¹H NMR (CDCl₃) δ 7.90 (dd, 1H), 7.75 (d, 1H), 6.93 (d, 1H), 5.08 (br s, 1H), 4.17 (t, 2H), 3.95 (s, 3H), 3.61 (q, 2H), 1.46 (s, 9H).

### Preparation of Intermediate I-9; 2-(tert-Butoxycarbonylamino)ethyl methanesulfonate

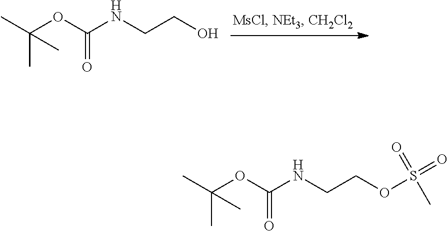

A solution of tert-butyl N-(2-hydroxyethyl)carbamate (1.068 g, 6.63 mmol) and Et₃N (1.1 mL, 7.9 mmol) in CH₂Cl₂ (30 mL) was cooled to 0° C. and treated with methanesulfonyl chloride (0.57 mL, 7.3 mmol). The reaction was allowed to slowly warm to rt and was stirred o/n. The reaction was diluted with CH₂Cl₂, washed with 5% NaOH and brine, dried (MgSO₄), filtered, and concentrated to provide the title compound. ¹H NMR (CDCl₃) δ 4.92 (br s, 1H), 4.29 (t, 2H), 3.48 (q, 2H), 3.04 (s, 3H), 1.45 (s, 9H).

### Preparation of Intermediate I-10; tert-Butyl N-(2-hydroxyethyl)carbamate

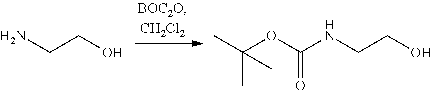

A solution of 2-aminoethanol (2.5 mL, 45.2 mmol) and Et₃N (5.9 mL, 915 mmol) in CH₂Cl₂ (100 mL) was treated with tert-butoxycarbonyl tert-butyl carbonate (11.5 mL) and stirred at rt o/n. The reaction was washed with brine, dried (MgSO₄) and concentrated to provide the title compound. ¹H NMR (CDCl₃) δ 4.99 (br s, 1H), 3.70 (br s, 2H), 3.30 (q, 2H), 2.67 (br s, 1H), 1.45 (s, 9H).

### Preparation of Intermediate I-11; tert-Butyl N-[4-[[4-(3-cyano-4-methoxy-phenyl)pyrimidin-2-yl]amino]phenyl]carbamate

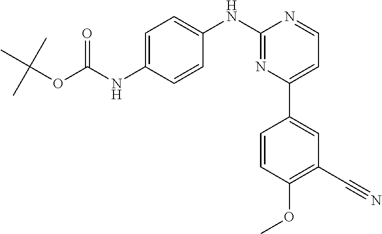 [SMILES: COc1ccc(-c2ccnc(Nc3ccc(NC(=O)OC(C)(C)C)cc3)n2)cc1C#N]

A mixture of 5-(2-chloropyrimidin-4-yl)-2-methoxy-benzonitrile (395 mg, 1.71 mmol), tert-butyl N-(4-aminophenyl)carbamate (396 mg, 1.9 mmol), Cs₂CO₃ (1.707 g, 5.24 mmol), BINAP (105 mg, 0.17 mmol) and Pd(OAc)₂ (22 mg, 0.098 mmol) in p-dioxane was refluxed for 3 h. The reaction was cooled to rt, diluted with H₂O, extracted with EtOAc, washed with brine, dried (MgSO₄), filtered, and concentrated. Purification by MPLC (SiO₂, EtOAc/Hexanes, 0-100%) provided the title compound. ¹H NMR (DMSO-d₆) δ 9.56 (s, 1H), 9.23 (br s, 1H), 8.55-8.45 (m, 3H), 7.69-7.64 (m, 2H), 7.46 (d, 1H), 7.43 (d, 1H), 7.43-7.34 (m, 2H), 4.01 (s, 3H), 1.48 (s, 9H).

### Preparation of Intermediate I-12; tert-Butyl N-[4-[[4-(3-cyano-4-tetrahydropyran-4-yloxy-phenyl)pyrimidin-2-yl]amino]phenyl]carbamate

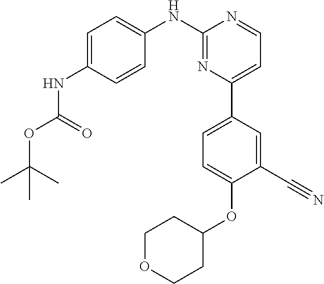 [SMILES: CC(C)(C)OC(=O)Nc1ccc(Nc2nccc(-c3ccc(OC4CCOCC4)c(C#N)c3)n2)cc1]

The procedure used for the preparation of Intermediate I-11 was used to prepare the title compound from 5-(2-chloropyrimidin-4-yl)-2-tetrahydropyran-4-yloxy-benzonitrile and tert-butyl N-(4-aminophenyl)carbamate. ¹H NMR (DMSO-d₆) δ 9.56 (s, 1H), 9.23 (br s, 1H), 8.53 (d, 1H), 8.51 (d, 1H), 8.44 (dd, 1H), 7.68-7.62 (m, 2H), 7.57 (d, 1H), 7.43 (d, 1H), 7.39 (d, 2H), 4.94 (sept, 1H), 3.92-3.82 (m, 2H), 3.55 (ddd, 2H), 2.12-2.00 (m, 2H), 1.78-1.60 (m, 2H), 1.48 (s, 9H).

### Preparation of Intermediate I-13; tert-Butyl N-[2-(2-hydroxyethoxy)ethyl]carbamate

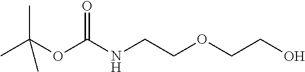

Di-tert-butyl dicarbonate (4.973 g, 22.8 mmol) in CHCl₃ (100 mL) was added dropwise to a solution of 2-(2-aminoethoxy)ethanol (2.4 mL, 22.8 mmol) in CHCl₃ (100 mL) and stirred o/n. Water was added and the layers separated. The aqueous layer was extracted once with CH₂Cl₂. The combined organics were dried (MgSO₄), filtered, and concentrated to provide the title compound. ¹H NMR (CDCl₃) δ 4.95 (br s, 1H), 3.78-3.70 (m, 2H), 3.60-3.52 (m, 4H), 3.38-3.28 (m, 2H), 2.22 (br s, 1H), 1.45 (s, 9H).

### Preparation of Intermediate I-14; 2-[2-(tert-Butoxycarbonylamino)ethoxy]ethyl methanesulfonate

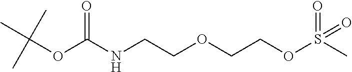 [SMILES: CC(C)(C)OC(=O)NCCOCCOS(C)(=O)=O]

Triethylamine (3.5 mL, 25.1 mmol) and methanesulfonyl chloride (1.90 mL, 24.5 mmol) were added to a 0° C. solution of tert-butyl N-[2-(2-hydroxyethoxy)ethyl]carbamate (22.8 mmol) in CH₂Cl₂ (100 mL). The reaction was warmed to rt and stirred for 1 h. Water was added and the layers separated. The organics were dried (MgSO₄), filtered, and concentrated to provide the title compound. ¹H NMR (CDCl₃) δ 4.93 (br s, 1H), 4.40-4.34 (m, 2H), 3.77-3.71 (m, 2H), 3.60-3.52 (m, 2H), 3.83-3.26 (m, 2H), 3.07 (s, 3H), 1.45 (s, 9H).

### Preparation of Intermediate I-15; tert-Butyl N-[2-[2-(2-methoxy-4-nitro-phenoxy)ethoxy]ethyl]carbamate

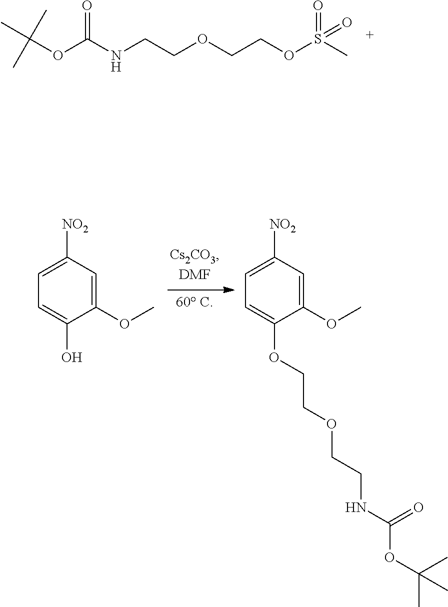 [SMILES: CC(C)(C)OC(=O)NCCOCCOS(C)(=O)=O ; COc1cc([N+](=O)[O-])ccc1O ; COc1cc([N+](=O)[O-])ccc1OCCOCCNC(=O)OC(C)(C)C]

Cesium carbonate (19.483 g, 60 mmol) and 2-[2-(tert-butoxycarbonylamino)ethoxy]ethyl methanesulfonate (4.353 g, 15.4 mmol) were added to a solution of 2-methoxy-4-nitro-phenol (2.005 g, 11.9 mmol) in DMF. The reaction was heated to 60° C. o/n. The reaction was cooled to rt, filtered and volatiles were removed via rotary evaporation. The residue was dissolved in EtOAc and washed with H₂O and brine. The combined aqueous layers were extracted once with EtOAc. The combined organics were dried (MgSO₄), filtered, and concentrated. Purification by MPLC (SiO₂, EtOAc/Hexanes, 0-100%) provided the title compound. ¹H NMR (CDCl₃) δ 7.90 (dd, 1H), 7.76 (d, 1H), 6.95 (d, 1H), 5.02 (br s, 1H), 4.26 (t, 2H), 3.96 (s, 3H), 3.92-3.87 (m, 2H), 3.62 (t, 2H), 3.39-3.30 (m, 2H), 1.44 (s, 9H).

### Preparation of Intermediate I-16; tert-Butyl N-[2-[2-[4-[[4-(3-cyano-4-tetrahydropyran-4-yloxy-phenyl)pyrimidin-2-yl]amino]-2-methoxy-phenoxy]ethoxy]ethyl]carbamate

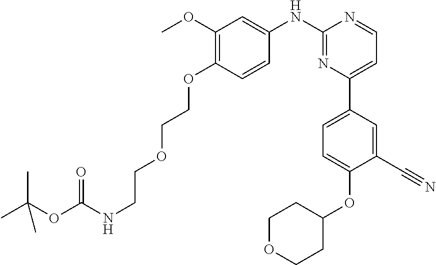 [SMILES: COc1cc(Nc2nccc(-c3ccc(OC4CCOCC4)c(C#N)c3)n2)ccc1OCCOCCNC(=O)OC(C)(C)C]

The procedure used in the preparation of Intermediate I-11 was used to prepare the title compound from 5-(2-chloropyrimidin-4-yl)-2-tetrahydropyran-4-yloxy-benzonitrile and tert-butyl N-[2-[2-(4-amino-2-methoxy-phenoxy)ethoxy]ethyl]carbamate. ¹H NMR (DMSO-d₆) δ 9.55 (s, 1H), 8.55 (d, 1H), 8.52 (d, 1H), 8.44 (dd, 1H), 7.62 (br s, 1H), 7.54 (d, 1H), 7.43 (d, 1H), 7.20 (d, 1H), 6.92 (d, 1H), 6.82 (t, 1H), 4.95 (sept, 1H), 4.07-3.98 (m, 2H), 3.92-3.84 (m, 2H), 3.81 (s, 3H), 3.74-3.66 (m, 2H), 3.56 (ddd, 2H), 3.46 (t, 2H), 3.10 (q, 2H), 2.10-2.00 (m, 2H), 1.76-1.64 (m, 2H), 1.38 (s, 9H).

### Preparation of Intermediate I-17; 1-(3-Chloropropylsulfonyl)-4-methyl-piperazine

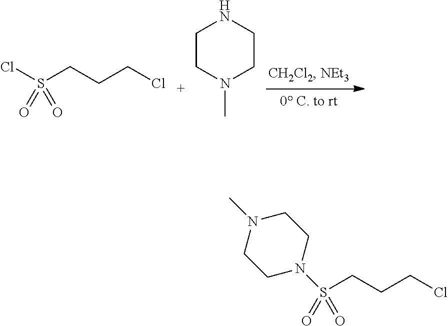

A solution of 3-chloropropane-1-sulfonyl chloride (170 μL, 1.4 mmol) in CH₂Cl₂ (2 mL) at 0° C. was treated with a solution of 1-methylpiperazine (170 μL, 1.5 mmol) and Et₃N (210 μL, 1.5 mmol) in CH₂Cl₂ (4 mL) and immediately allowed to warm to rt. After 2 h the reaction was concentrated. Ethyl acetate was added and the resulting suspension filtered. The filtrate was concentrated to provide the title compound. ¹H NMR (CDCl₃) δ 3.72-3.68 (m, 2H), 3.39-3.32 (m, 4H), 3.12-3.06 (m, 2H), 2.58-2.50 (m, 4H), 2.36 (s, 3H), 2.34-2.26 (m, 2H).

### Preparation of Intermediate I-18; 1-[3-(2-Methoxy-4-nitro-phenoxy)propylsulfonyl]-4-methyl-piperazine

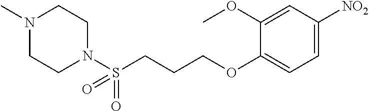 [SMILES: COc1cc([N+](=O)[O-])ccc1OCCCS(=O)(=O)N1CCN(C)CC1]

The procedure used in the preparation of Intermediate 1-15 was used to prepare the title compound from 1-(3-chloropropylsulfonyl)-4-methyl-piperazine and 2-methoxy-4-nitro-phenol. ¹H NMR (CDCl₃) δ 7.90 (dd, 1H), 7.75 (d, 1H), 7.91 (d, 1H), 4.25 (t, 2H), 3.94 (s, 3H), 3.37-3.30 (m, 4H), 3.19-3.12 (m, 2H), 2.54-2.46 (m, 4H), 2.45-2.35 (m, 2H), 2.33 (s, 3H).

### Preparation of Intermediate I-19; 3-Methoxy-4-[3-(4-methylpiperazin-1-yl)sulfonylpropoxy]aniline

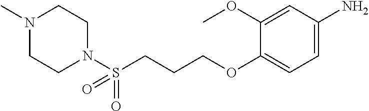 [SMILES: COc1cc(N)ccc1OCCCS(=O)(=O)N1CCN(C)CC1]

The procedure used in the preparation of Intermediate I-7 was used to prepare the title compound from 1-[3-(2-methoxy-4-nitro-phenoxy)propylsulfonyl]-4-methyl-piperazine. ¹H NMR (CDCl₃) δ 6.73 (d, 1H), 6.29 (d, 1H), 6.20 (dd, 1H), 4.04 (t, 2H), 3.80 (s, 3H), 3.49 (br s, 2H), 3.36-3.28 (m, 4H), 3.21-3.14 (m, 2H), 2.53-2.44 (m, 4H), 2.32 (s, 3H), 2.28-2.20 (m, 2H).

### Preparation of Intermediate I-20; 4-(3-Chloropropylsulfonyl)morpholine

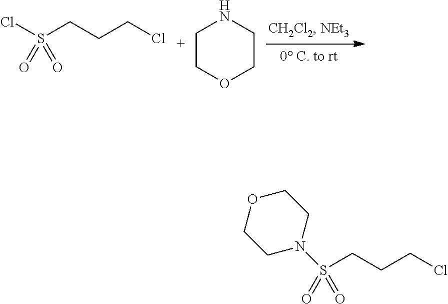

A solution of 3-chloropropane-1-sulfonyl chloride (170 μL, 1.4 mmol) in CH₂Cl₂ (2 mL) at 0° C. was treated with a solution of morpholine (140 μL, 1.6 mmol) and Et₃N (210 μL, 1.5 mmol) in CH₂Cl₂ (4 mL) and immediately allowed to warm to rt. After 2 h the reaction was concentrated. Ethyl acetate was added and a resulting precipitate was filtered. The filtrate was concentrated to provide the title compound. ¹H NMR (CDCl₃) δ 3.81-3.76 (m, 4H), 3.73-3.68 (m, 2H), 3.33-3.26 (m, 4H), 3.14-3.06 (m, 2H), 2.38-2.26 (m, 2H).

### Preparation of Intermediate I-21; 4-Amino-2-methoxy-phenol

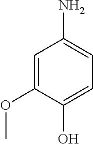

The procedure used in the preparation of Intermediate I-7 was used to prepare the title compound from 4-nitro-2-methoxy-phenol. ¹H NMR (CDCl₃) δ 7.81 (s, 1H), 6.45 (d, 1H), 6.22 (d, 1H), 5.98 (dd, 1H), 4.45 (br s, 2H), 3.66 (s, 3H).

### Preparation of Intermediate I-22; tert-Butyl N-[2-(4-amino-2-methoxy-phenoxy)ethyl]carbamate

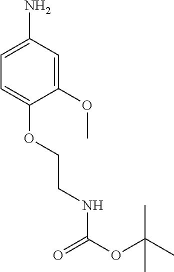 [SMILES: COc1cc(N)ccc1OCCNC(=O)OC(C)(C)C]

The procedure used in the preparation of Intermediate I-7 was used to prepare the title compound from tert-butyl N-[2-(2-methoxy-4-nitro-phenoxy)ethyl]carbamate. ¹H NMR (CDCl₃) δ 6.76 (d, 1H), 6.35 (d, 1H), 6.27 (dd, 1H), 3.99 (t, 2H), 3.83 (s, 3H), 3.52-3.42 (m, 2H), 1.45 (s, 9H).

### Preparation of Intermediate I-23; tert-Butyl N-[2-[4-[[4-(3-cyano-4-tetrahydropyran-4-yloxy-phenyl)pyrimidin-2-yl]amino]-2-methoxy-phenoxy]ethyl]carbamate

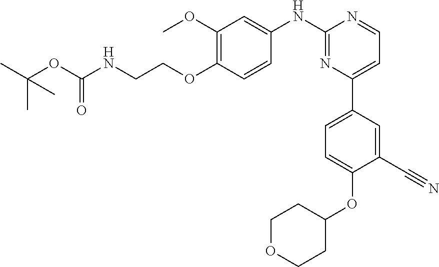 [SMILES: COc1cc(Nc2nccc(-c3ccc(OC4CCOCC4)c(C#N)c3)n2)ccc1OCCNC(=O)OC(C)(C)C]

The procedure used in the preparation of Intermediate I-11 was used to prepare the title compound from 5-(2-chloropyrimidin-4-yl)-2-tetrahydropyran-4-yloxy-benzonitrile and tert-butyl N-[2-(4-amino-2-methoxy-phenoxy)ethyl]carbamate. ¹H NMR (CDCl₃) δ 8.46 (d, 1H), 8.36 (d, 1H), 8.22 (dd, 1H), 7.71-7.63 (m, 2H), 7.58-7.50 (m, 2H), 7.45-7.50 (m, 2H), 4.76 (sept, 1H), 4.11-4.00 (m, 6H), 3.95 (s, 3H), 3.70-3.62 (m, 2H), 3.58-3.48 (m, 2H), 2.14-2.04 (m, 2H), 1.59 (s, 9H).

### Preparation of Intermediate I-24; tert-Butyl N-[2-[2-(2-methoxy-5-nitro-phenoxy)ethoxy]ethyl]carbamate

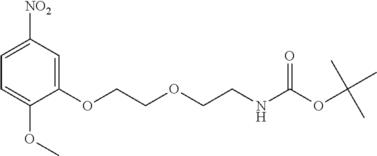 [SMILES: COc1ccc([N+](=O)[O-])cc1OCCOCCNC(=O)OC(C)(C)C]

The procedure used in the preparation of Intermediate I-8 was used to prepare the title compound from 2-[2-(tert-butoxycarbonylamino)ethoxy]ethyl methanesulfonate and 2-methoxy-5-nitro-phenol. ¹H NMR (CDCl₃) δ 7.93 (dd, 1H), 7.86-7.78 (m, 1H), 6.92 (d, 1H), 5.07 (br s, 1H), 4.28-4.25 (m, 2H), 3.98 (s, 3H), 3.92-3.82 (m, 2H), 3.63 (t, 2H), 3.35 (q, 2H), 1.43 (s, 9H).

### Preparation of Intermediate I-25; 4-[3-(2-Methoxy-4-nitro-phenoxy)propyl]morpholine

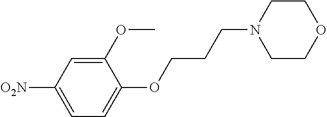 [SMILES: COc1cc([N+](=O)[O-])ccc1OCCCN1CCOCC1]

The procedure used in the preparation of Intermediate I-8 was used to prepare the title compound from 3-morpholinopropyl methanesulfonate and 2-methoxy-4-nitro-phenol. ¹H NMR (CDCl₃) δ 7.90 (dd, 1H), 7.74 (d, 1H), 6.94 (d, 1H), 4.20 (t, 2H), 3.95 (s, 3H), 3.72 (t, 4H), 2.54 (t, 2H), 2.51 (br s, 4H), 2.07 (quint, 2H).

### Preparation of Intermediate I-26; tert-Butyl N-[2-[2-(5-amino-2-methoxy-phenoxy)ethoxy]ethyl]carbamate

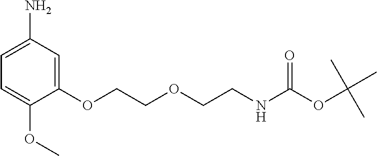 [SMILES: COc1ccc(N)cc1OCCOCCNC(=O)OC(C)(C)C]

The procedure used in the preparation of Intermediate I-7 was used to prepare the title compound from tert-butyl N-[2-[2-(2-methoxy-5-nitro-phenoxy)ethoxy]ethyl]carbamate. The title compound was purified by MPLC (SiO₂, EtOAc/Hexanes, 0-100%). ¹H NMR (CDCl₃) δ 6.72 (d, 1H), 6.36 (d, 1H), 6.26 (dd, 1H), 5.10 (br s, 1H), 4.16-4.08 (m, 2H), 3.85-3.80 (m, 2H), 3.79 (s, 3H), 3.60 (t, 2H), 3.46 (br s, 2H), 3.34 (q, 2H), 1.44 (s, 9H).

### Preparation of Intermediate I-27; 3-Methoxy-4-(3-morpholinopropoxy)aniline

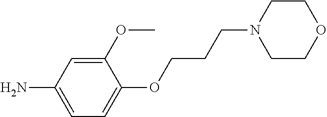 [SMILES: COc1cc(N)ccc1OCCCN1CCOCC1]

The procedure used in the preparation of Intermediate I-7 was used to prepare the title compound from 4-[3-(2-methoxy-4-nitro-phenoxy)propyl]morpholine. ¹H NMR (CDCl₃) δ 6.75 (d, 1H), 6.31 (d, 1H), 6.21 (dd, 1H), 3.99 (t, 2H), 3.83 (s, 3H), 3.78-3.70 (m, 4H), 2.62-2.44 (m, 4H), 2.04-1.96 (m, 2H).

### Preparation of Intermediate I-28; tert-Butyl N-[2-[2-[5-[[4-(3-cyano-4-tetrahydropyran-4-yloxy-phenyl)pyrimidin-2-yl]amino]-2-methoxy-phenoxy]ethoxy]ethyl]carbamate

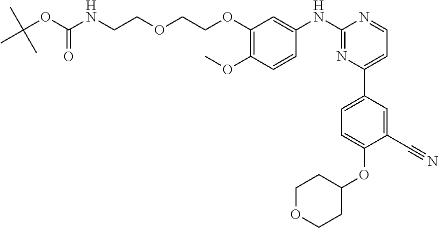 [SMILES: COc1ccc(Nc2nccc(-c3ccc(OC4CCOCC4)c(C#N)c3)n2)cc1OCCOCCNC(=O)OC(C)(C)C]

The procedure used in the preparation of Intermediate I-11 was used to prepare the title compound from 5-(2-chloropyrimidin-4-yl)-2-tetrahydropyran-4-yloxy-benzonitrile and tert-butyl N-[2-[2-(5-amino-2-methoxy-phenoxy)ethoxy]ethyl]carbamate. ¹H NMR (DMSO-d₆) δ 9.51 (s, 1H), 8.54 (d, 1H), 8.52 (d, 1H), 8.44 (dd, 1H), 7.54 (d, 1H), 7.42 (d, 1H), 7.26 (dd, 1H), 6.92 (d, 1H), 6.84-6.75 (m, 1H), 4.94 (sept, 1H), 4.14-4.05 (m, 2H), 3.92-3.83 (m, 2H), 3.78-3.72 (m, 2H), 3.74 (s, 3H), 3.56 (ddd, 2H), 3.46 (t, 2H), 3.10 (q, 2H), 2.10-2.00 (m, 2H), 1.75-1.62 (m, 2H), 1.36 (s, 9H).

### Preparation of Intermediate I-29; 5-{2-[(4-Aminophenyl)amino]pyrimidin-4-yl}-2-(tetrahydro-2H-pyran-4-yloxy)benzonitrile

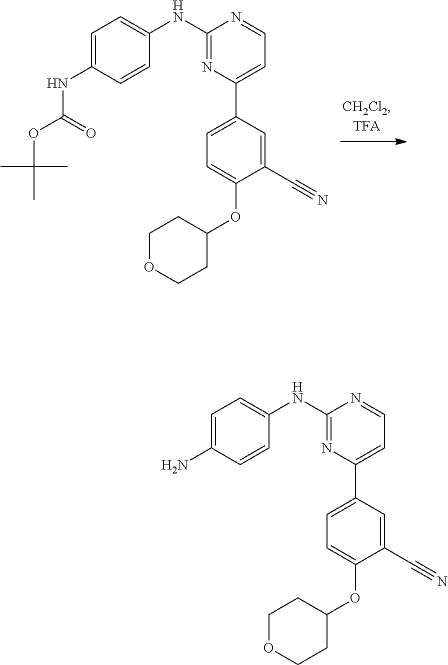 [SMILES: CC(C)(C)OC(=O)Nc1ccc(Nc2nccc(-c3ccc(OC4CCOCC4)c(C#N)c3)n2)cc1 ; N#Cc1cc(-c2ccnc(Nc3ccc(N)cc3)n2)ccc1OC1CCOCC1]

A solution of tert-butyl N-[4-[[4-(3-cyano-4-tetrahydropyran-4-yloxy-phenyl) pyrimidin-2-yl]amino]phenyl]carbamate in CH₂Cl₂ was treated with TFA (10% by volume) and stirred for 1.5 h. The reaction was quenched with NaHCO₃ (saturated (sat.), aq.) and the mixture extracted with EtOAc. The combined organics were dried (MgSO₄), filtered, and concentrated to provide the title compound. ¹H NMR (DMSO-d₆) δ 9.18 (s, 1H), 8.49 (d, 1H), 8.43 (d, 1H), 8.40 (dd, 1H), 7.53 (d, 1H), 7.36-7.30 (m, 2H), 7.32 (d, 1H), 6.58-6.54 (m, 2H), 4.93 (sept, 1H), 1.82 (br s, 2H), 3.92-3.82 (m, 2H), 3.55 (ddd, 2H), 2.10-2.00 (m, 2H), 1.75-1.62 (m, 2H); LC-MS [M+H]⁺ 388.1763.

### Preparation of Intermediate I-30; 5-{2-[(4-Hydroxy-3-methoxyphenyl)amino]pyrimidin-4-yl}-2-(tetrahydro-2H-pyran-4-yloxy)benzonitrile

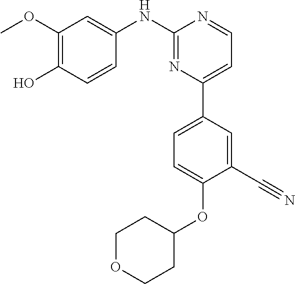 [SMILES: COc1cc(Nc2nccc(-c3ccc(OC4CCOCC4)c(C#N)c3)n2)ccc1O]

The procedure used for the preparation of Intermediate I-11 was used to prepare the title compound from 5-(2-chloropyrimidin-4-yl)-2-tetrahydropyran-4-yloxy-benzonitrile and 4-amino-2-methoxy-phenol. ¹H NMR (DMSO-d₆) 9.42 (s, 1H), 8.62 (s, 1H), 8.55 (d, 1H), 8.49 (d, 1H), 8.43 (dd, 1H), 7.56 (br s, 1H), 7.54 (d, 1H), 7.39 (d, 1H), 7.05 (d, 1H), 6.71 (d, 1H), 4.95 (sept, 1H), 3.92-3.83 (m, 2H), 3.81 (s, 3H), 3.55 (ddd, 2H), 2.10-2.00 (m, 2H), 1.63-1.75 (m, 2H); LC-MS [M+H]⁺ 419.1718.

### Preparation of Intermediate I-31; tert-Butyl N-[2-[2-[4-[[4-(3-cyano-4-tetrahydropyran-4-yloxy-phenyl)pyrimidin-2-yl]amino]-2-methoxy-phenoxy]ethoxy]ethyl]carbamate

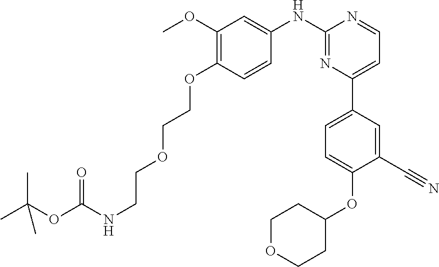 [SMILES: COc1cc(Nc2nccc(-c3ccc(OC4CCOCC4)c(C#N)c3)n2)ccc1OCCOCCNC(=O)OC(C)(C)C]

The procedure used for the preparation of Intermediate I-11 was used to prepare the title compound from 5-(2-chloropyrimidin-4-yl)-2-tetrahydropyran-4-yloxy-benzonitrile and tert-butyl N-[2-[2-(4-amino-2-methoxy-phenoxy)ethoxy]ethyl]carbamate. ¹H NMR (DMSO-d₆) δ 9.55 (s, 1H), 8.55 (d, 1H), 8.52 (d, 1H), 8.44 (dd, 1H), 7.62 (br s, 1H), 7.54 (d, 1H), 7.43 (d, 1H), 7.20 (d, 1H), 6.92 (d, 1H), 6.82 (t, 1H), 4.95 (sept, 1H), 4.07-3.98 (m, 2H), 3.92-3.84 (m, 2H), 3.81 (s, 3H), 3.74-3.66 (m, 2H), 3.56 (ddd, 2H), 3.46 (t, 2H), 3.10 (q, 2H), 2.10-2.00 (m, 2H), 1.76-1.64 (m, 2H), 1.38 (s, 9H).

### Preparation of Intermediate I-32; 5-[2-[(4-Morpholinophenyl)amino]pyrimidin-4-yl]-2-(4-piperidylmethoxy)benzonitrile

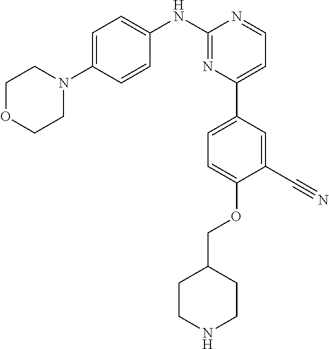 [SMILES: N#Cc1cc(-c2ccnc(Nc3ccc(N4CCOCC4)cc3)n2)ccc1OCC1CCNCC1]

The procedures used for the preparation of Intermediate I-5 followed by the procedure for Intermediate I-6 were used to prepare the title compound from tert-butyl 4-(hydroxymethyl)piperidine-1-carboxylate. ¹H NMR (CDCl₃) δ 8.43 (d, 1H), 8.29 (d, 1H), 8.23-8.21 (m, 1H), 7.56-7.53 (m, 2H), 7.15 (s, 1H), 7.06-6.94 (m, 4H), 3.95 (d, 2H), 3.90-3.87 (m, 4H), 3.18-3.13 (m, 6H), 2.72-2.64 (m, 2H), 2.12-2.02 (m, 1H), 1.94-1.87 (m, 2H), 1.36-1.25 (m, 2H). TOF LC-MS [M+H]⁺ 471.2403.

### Preparation of Intermediate I-33; tert-Butyl 3-[2-cyano-4-[2-[(4-morpholinophenyl)amino]pyrimidin-4-yl]phenoxy]azetidine-1-carboxylate

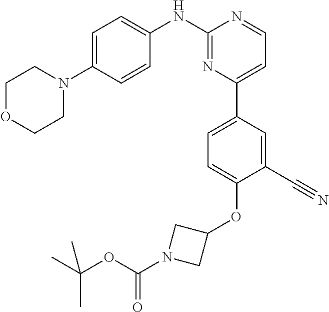 [SMILES: CC(C)(C)OC(=O)N1CC(Oc2ccc(-c3ccnc(Nc4ccc(N5CCOCC5)cc4)n3)cc2C#N)C1]

The procedure used for the preparation of Intermediate I-5 was used to prepare the title compound from tert-butyl 3-hydroxyazetidine-1-carboxylate. ¹H NMR (DMSO-d₆) δ 9.48 (s, 1H), 8.55 (d, 1H), 8.50 (d, 1H), 8.44-8.41 (m, 1H), 7.64-7.62 (m, 2H), 7.40 (d, 1H), 7.16 (d, 1H), 6.94-6.91 (m, 2H), 5.27-5.21 (m, 1H), 4.43-4.36 (m, 2H), 3.93-3.87 (m, 2H), 3.76-3.73 (m, 4H), 3.06-3.03 (m, 4H), 1.40 (s, 9H). TOF LC-MS [M+H]⁺ 529.2522.

### Preparation of Intermediate I-34; 2-(Azetidin-3-yloxy)-5-[2-[(4-morpholinophenyl)amino]pyrimidin-4-yl]benzonitrile

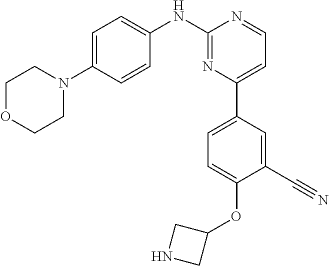 [SMILES: N#Cc1cc(-c2ccnc(Nc3ccc(N4CCOCC4)cc3)n2)ccc1OC1CNC1]

The procedure used for the preparation of Intermediate I-6 was used to prepare the title compound from tert-butyl 3-hydroxyazetidine-1-carboxylate. ¹H NMR (DMSO-d₆) δ 9.46 (s, 1H), 8.53 (d, 1H), 8.48 (d, 1H), 8.42-8.39 (m, 1H), 7.65-7.62 (m, 2H), 7.37 (d, 1H), 7.13 (d, 1H), 6.92 (d, 2H), 5.27-5.20 (m, 1H), 3.88-3.83 (m, 2H), 3.76-3.73 (m, 4H), 3.60-3.55 (m, 2H), 3.34 (br s, 1H), 3.06-3.03 (m, 4H). TOF LC-MS [M+H]⁺ 429.1945.

### Preparation of Intermediate I-35; 2-(2-aminoethoxy)-5-[2-[(4-morpholinophenyl)amino]pyrimidin-4-yl]benzonitrile

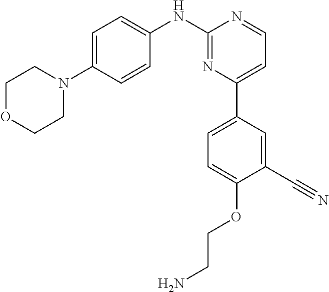 [SMILES: N#Cc1cc(-c2ccnc(Nc3ccc(N4CCOCC4)cc3)n2)ccc1OCCN]

The procedures used for the preparation of Intermediate I-5 followed by the procedure for Intermediate I-6 were used to prepare the title compound from tert-butyl N-(2-hydroxyethyl)carbamate. ¹H NMR (DMSO-d₆) δ 9.46 (s, 1H), 8.52-8.43 (m, 3H), 7.65-7.62 (m, 2H), 7.44 (d, 1H), 7.39 (d, 1H), 6.93 (d, 2H), 4.19 (t, 2H), 3.76-3.73 (m, 4H), 3.06-3.03 (m, 4H), 2.96 (t, 2H). TOF LC-MS [M+H]⁺ 416.1901.

### Preparation of Intermediate I-36; tert-Butyl N-[4-[[4-(3-cyano-4-methoxy-phenyl)pyrimidin-2-yl]amino]phenyl]carbamate

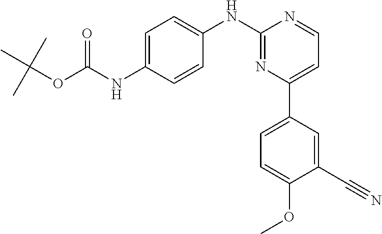 [SMILES: COc1ccc(-c2ccnc(Nc3ccc(NC(=O)OC(C)(C)C)cc3)n2)cc1C#N]

The procedure used for the preparation of Intermediate I-11 was used to prepare the title compound from 5-(2-chloropyrimidin-4-yl)-2-methoxy-benzonitrile and tert-butyl N-(4-aminophenyl)carbamate. ¹H NMR (DMSO-d₆) δ 9.56 (s, 1H), 9.23 (br s, 1H), 8.55-8.45 (m, 3H), 7.69-7.64 (m, 2H), 7.46 (d, 1H), 7.43 (d, 1H), 7.43-7.34 (m, 2H), 4.01 (s, 3H), 1.48 (s, 9H).

### Preparation of Intermediate I-37; tert-Butyl N-[2-[2-[4-[[4-(3-cyano-4-tetrahydropyran-4-yloxy-phenyl)pyrimidin-2-yl]amino]-2-methoxy-phenoxy]ethoxy]ethyl]carbamate

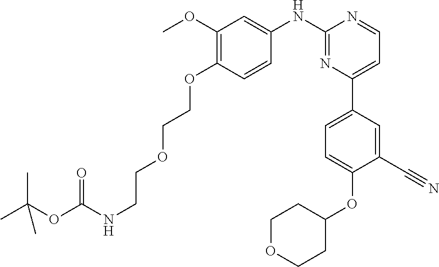 [SMILES: COc1cc(Nc2nccc(-c3ccc(OC4CCOCC4)c(C#N)c3)n2)ccc1OCCOCCNC(=O)OC(C)(C)C]

The procedure used for the preparation of Intermediate I-11 was used to prepare the title compound from tert-butyl N-[2-[2-(4-amino-2-methoxy-phenoxy)ethoxy]ethyl]carbamate and 5-(2-chloropyrimidin-4-yl)-2-tetrahydropyran-4-yloxy-benzonitrile. ¹H NMR (DMSO-d₆) δ 9.55 (s, 1H), 8.55 (d, 1H), 8.52 (d, 1H), 8.44 (dd, 1H), 7.62 (br s, 1H), 7.54 (d, 1H), 7.43 (d, 1H), 7.20 (d, 1H), 6.92 (d, 1H), 6.82 (t, 1H), 4.95 (sept, 1H), 4.07-3.98 (m, 2H), 3.92-3.84 (m, 2H), 3.81 (s, 3H), 3.74-3.66 (m, 2H), 3.56 (ddd, 2H), 3.46 (t, 2H), 3.10 (q, 2H), 2.10-2.00 (m, 2H), 1.76-1.64 (m, 2H), 1.38 (s, 9H).

### Preparation of Intermediate I-38; tert-Butyl N-[2-[4-[[4-(3-cyano-4-tetrahydropyran-4-yloxy-phenyl)pyrimidin-2-yl]amino]-2-methoxy-phenoxy]ethyl]carbamate

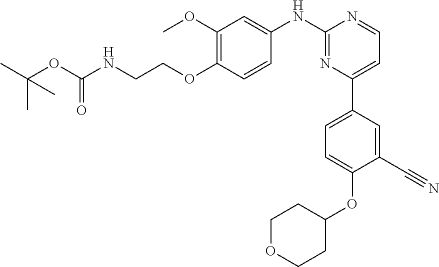 [SMILES: COc1cc(Nc2nccc(-c3ccc(OC4CCOCC4)c(C#N)c3)n2)ccc1OCCNC(=O)OC(C)(C)C]

The procedure used for the preparation of Intermediate I-11 was used to prepare the title compound from 5-(2-chloropyrimidin-4-yl)-2-tetrahydropyran-4-yloxy-benzonitrile and tert-butyl N-[2-(4-amino-2-methoxy-phenoxy)ethyl]carbamate. ¹H NMR (CDCl₃) δ 8.46 (d, 1H), 8.36 (d, 1H), 8.22 (dd, 1H), 7.71-7.63 (m, 2H), 7.58-7.50 (m, 2H), 7.45-7.50 (m, 2H), 4.76 (sept, 1H), 4.11-4.00 (m, 6H), 3.95 (s, 3H), 3.70-3.62 (m, 2H), 3.58-3.48 (m, 2H), 2.14-2.04 (m, 2H), 1.59 (s, 9H).

### Preparation of Intermediate I-39; tert-Butyl N-[2-[2-[5-[[4-(3-cyano-4-tetrahydropyran-4-yloxy-phenyl)pyrimidin-2-yl]amino]-2-methoxy-phenoxy]ethoxy]ethyl]carbamate

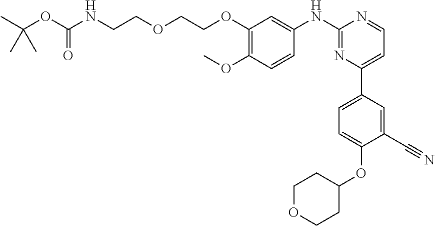 [SMILES: COc1ccc(Nc2nccc(-c3ccc(OC4CCOCC4)c(C#N)c3)n2)cc1OCCOCCNC(=O)OC(C)(C)C]

The procedure used for the preparation of Intermediate I-11 was used to prepare the title compound from 5-(2-chloropyrimidin-4-yl)-2-tetrahydropyran-4-yloxy-benzonitrile and tert-butyl N-[2-[2-(5-amino-2-methoxy-phenoxy)ethoxy]ethyl]carbamate. ¹H NMR (DMSO-d₆) δ 9.51 (s, 1H), 8.54 (d, 1H), 8.52 (d, 1H), 8.44 (dd, 1H), 7.54 (d, 1H), 7.42 (d, 1H), 7.26 (dd, 1H), 6.92 (d, 1H), 6.84-6.75 (m, 1H), 4.94 (sept, 1H), 4.14-4.05 (m, 2H), 3.92-3.83 (m, 2H), 3.78-3.72 (m, 2H), 3.74 (s, 3H), 3.56 (ddd, 2H), 3.46 (t, 2H), 3.10 (q, 2H), 2.10-2.00 (m, 2H), 1.75-1.62 (m, 2H), 1.36 (s, 9H).

### Preparation of Intermediate I-40; Methyl 4-[[4-(3-cyano-4-tetrahydropyran-4-yloxy-phenyl)pyrimidin-2-yl]amino]-2-methoxy-benzoate

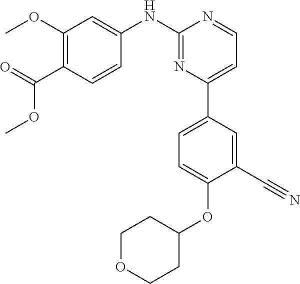 [SMILES: COC(=O)c1ccc(Nc2nccc(-c3ccc(OC4CCOCC4)c(C#N)c3)n2)cc1OC]

The procedure used for the preparation of Intermediate I-11 was used to prepare the title compound from methyl 4-amino-2-methoxy-benzoate and 5-(2-chloropyrimidin-4-yl)-2-(tetrahydro-2H-pyran-4-yloxy)benzonitrile. ¹H NMR (DMSO-d₆) δ 10.1 (br s, 1H), 8.62 (d, 2H), 8.48 (s, 1H), 7.90 (s, 1H), 7.72 (d, 1H), 7.59 (t, 2H), 7.39 (d, 1H), 4.96 (m, 1H), 3.90-3.86 (m, 2H), 3.88 (s, 3H), 3.76 (s, 3H), 3.57 (m, 2H), 2.04 (m, 2H), 1.69 (m, 2H); LC-MS [M+H]⁺ 461.

### Preparation of Intermediate I-41; 4-[[4-(3-Cyano-4-tetrahydropyran-4-yloxy-phenyl)pyrimidin-2-yl]amino]-2-methoxy-benzoic acid

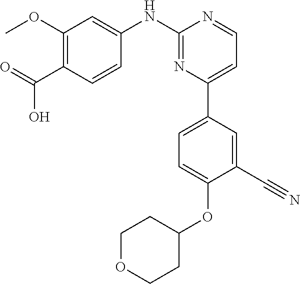 [SMILES: COc1cc(Nc2nccc(-c3ccc(OC4CCOCC4)c(C#N)c3)n2)ccc1C(=O)O]

The Standard Method G; Ester Hydrolysis procedure was used to prepare the title compound from methyl 4-[[4-(3-cyano-4-tetrahydropyran-4-yloxy-phenyl)pyrimidin-2-yl]amino]-2-methoxy-benzoate. ¹H NMR (DMSO-d₆) δ 12.0 (br s, 1H), 10.0 (s, 1H), 8.61 (dd, 2H), 8.48 (d, 1H), 7.89 (s, 1H), 7.71 (d, 1H), 7.60-7.56 (m, 2H), 7.36 (dd, 1H), 4.96 (m, 1H), 3.89-3.85 (m, 2H), 3.87 (s, 3H), 3.58-3.53 (m, 2H), 2.07-2.04 (m, 2H), 1.71-1.66 (m, 2H); LC-MS [M+H]⁺ 447.

### Preparation of Intermediate I-42; tert-Butyl 4-[2-cyano-4-[2-[(4-methoxycarbonylphenyl)amino]pyrimidin-4-yl]phenoxy]piperidine-1-carboxylate

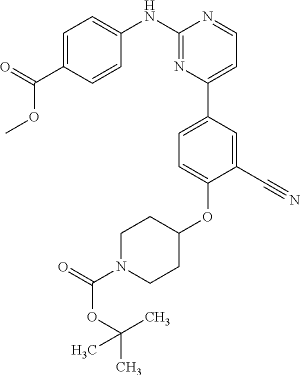 [SMILES: COC(=O)c1ccc(Nc2nccc(-c3ccc(OC4CCN(C(=O)OC(C)(C)C)CC4)c(C#N)c3)n2)cc1]

The procedure used for the preparation of Intermediate I-11 was used to prepare the title compound from methyl 4-amino-benzoate and tert-butyl 4-[4-(2-chloropyrimidin-4-yl)-2-cyanophenoxy]piperidine-1-carboxylate. ¹H NMR (DMSO-d₆) δ 10.2 (s, 1H), 8.64 (d, 1H), 8.58 (d, 1H), 8.50 (dd, 1H), 7.99-7.91 (m, 4H), 7.65-7.56 (m, 2H), 4.98-4.92 (m, 1H), 3.83 (s, 3H), 3.65-3.56 (m, 2H), 3.36-3.29 (m, 2H), 2.01-1.93 (m, 2H), 1.72-1.62 (m, 2H), 1.42 (s, 9H); LC-MS [M+H]⁺ 530.3.

### Preparation of Intermediate I-43; Methyl 4-[[4-[3-cyano-4-(4-piperidyloxy)phenyl]pyrimidin-2-yl]amino]benzoate

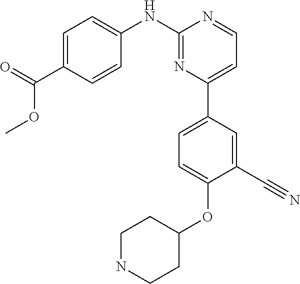 [SMILES: COC(=O)c1ccc(Nc2nccc(-c3ccc(OC4CCNCC4)c(C#N)c3)n2)cc1]

The Standard Method E; BOC Deprotection procedure was used to prepare the title compound from tert-butyl 4-[2-cyano-4-[2-[(4-methoxycarbonylphenyl)amino]pyrimidin-4-yl]phenoxy]piperidine-1-carboxylate. ¹H NMR (DMSO-d₆) δ 10.2 (s, 1H), 8.65 (d, 1H), 8.60 (d, 1H), 8.51 (dd, 1H), 7.98-7.92 (m, 4H), 7.61-7.56 (m, 2H), 4.99-4.93 (m, 1H), 3.83 (s, 3H), 3.27-3.19 (m, 2H), 3.16-3.09 (m, 2H), 2.19-2.11 (m, 2H), 1.97-1.87 (m, 2H); LC-MS [M+H]⁺ 430.2.

### Preparation of Intermediate I-44; Methyl 4-[[4-[3-cyano-4-[[1-[(2R)-2-hydroxypropanoyl]-4-piperidyl]oxy]phenyl]pyrimidin-2-yl]amino]benzoate

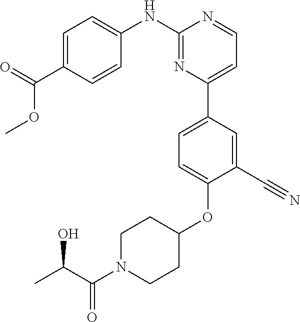 [SMILES: COC(=O)c1ccc(Nc2nccc(-c3ccc(OC4CCN(C(=O)[C@@H](C)O)CC4)c(C#N)c3)n2)cc1]

The Standard Method H; HATU Coupling procedure was used to prepare the title compound from lactic acid and methyl 4-[[4-[3-cyano-4-(4-piperidyloxy)phenyl]pyrimidin-2-yl]amino]benzoate. ¹H NMR (DMSO-d₆) δ 10.2 (s, 1H), 8.64 (d, 1H), 8.58 (d, 1H), 8.53-8.49 (m, 1H), 8.00-7.92 (m, 4H), 7.61-7.58 (m, 2H), 5.06-4.95 (m, 1H), 4.51-4.44 (m, 1H), 3.83 (s, 3H), 3.78-3.68 (m, 2H), 3.58-3.46 (m, 2H), 2.08-1.92 (m, 2H), 1.80-1.65 (m, 2H), 1.25 (d, 3H); LC-MS [M+H]⁺ 502.2.

### Preparation of Intermediate I-45; tert-Butyl 4-[3-[5-[[4-(3-cyano-4-tetrahydropyran-4-yloxy-phenyl)pyrimidin-2-yl]amino]-2-methoxy-phenoxy]propyl]piperazine-1-carboxylate

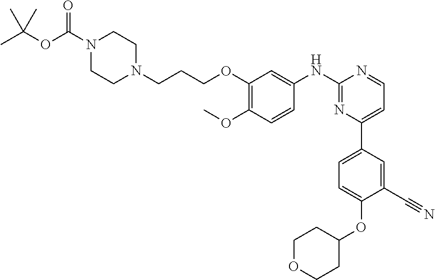 [SMILES: COc1ccc(Nc2nccc(-c3ccc(OC4CCOCC4)c(C#N)c3)n2)cc1OCCCN1CCN(C(=O)OC(C)(C)C)CC1]

The procedure used for the preparation of Intermediate I-11 was used to prepare the title compound from tert-butyl 4-[3-(5-amino-2-methoxy-phenoxy)propyl]piperazine-1-carboxylate and 5-(2-chloropyrimidin-4-yl)-2-tetrahydropyran-4-yloxy-benzonitrile. ¹H NMR (DMSO-d₆) δ 9.50 (s, 1H), 8.53 (d, 1H), 8.51 (d, 1H), 8.42 (dd, 1H), 7.59 (br s, 1H), 7.54 (d, 1H), 7.41 (d, 1H), 7.25-7.20 (m, 1H), 6.90 (d, 1H), 4.94 (sept., 1H), 4.18-3.98 (m, 2H), 3.92-3.82 (m, 2H), 3.73 (s, 3H), 3.55 (ddd, 2H), 3.30-3.22 (m, 4H), 2.47-2.40 (m, 2H), 2.32-2.26 (m, 4H), 2.08-2.00 (m, 2H), 1.95-1.84 (m, 2H), 1.75-1.62 (m, 2H), 1.39 (s, 9H).

### Preparation of Intermediate I-46; tert-Butyl 4-[3-[5-[[4-(3-cyano-4-tetrahydropyran-4-yloxy-phenyl)pyrimidin-2-yl]amino]-2-methoxy-phenoxy]propyl]piperidine-1-carboxylate

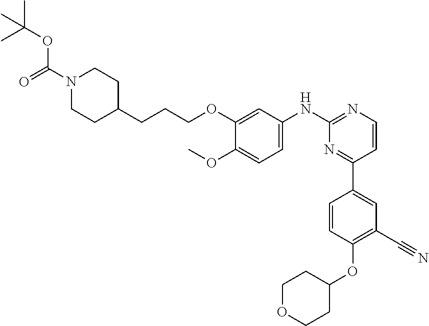 [SMILES: COc1ccc(Nc2nccc(-c3ccc(OC4CCOCC4)c(C#N)c3)n2)cc1OCCCC1CCN(C(=O)OC(C)(C)C)CC1]

The procedure used for the preparation of Intermediate I-11 was used to prepare the title compound from tert-butyl 4-[3-(5-amino-2-methoxy-phenoxy)propyl]piperidine-1-carboxylate and 5-(2-chloropyrimidin-4-yl)-2-tetrahydropyran-4-yloxy-benzonitrile. ¹H NMR (DMSO-d₆) δ 9.51 (s, 1H), 8.53 (d, 1H), 8.51 (d, 1H), 8.43 (dd, 1H), 7.63 (s, 1H), 7.56 (d, 1H), 7.41 (d, 1H), 7.19 (d, 1H), 6.90 (d, 1H), 4.94 (sept., 1H), 4.00-3.82 (m, 6H), 3.73 (s, 3H), 3.55 (d, 2H), 2.80-2.60 (m, 2H), 2.10-1.98 (m, 2H), 1.80-1.58 (m, 6H), 1.39 (s, 9H), 1.43-1.28 (m, 3H), 1.10-0.98 (m, 2H).

### Preparation of Intermediate I-47; 2,4-Dichloroquinazoline

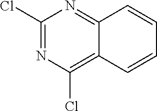 [SMILES: Clc1nc(Cl)c2ccccc2n1]

A mixture of 1H-quinazoline-2,4-dione (2.850 g, 17.5 mmol), dimethylaminopyridine (1.6 mL) in POCl₃ (8 mL) was refluxed for 4 h. The resulting solution was poured onto ice and the product collected via filtration. ¹H NMR (DMSO-d₆) 8.35-8.30 (m, 1H), 8.19 (ddd, 1H), 8.09-8.04 (m, 1H), 7.93 (ddd, 1H).

### Preparation of Intermediate I-48; 3-(2-Chloroquinazolin-4-yl)benzonitrile

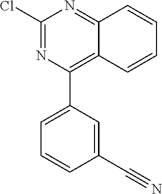 [SMILES: N#Cc1cccc(-c2nc(Cl)nc3ccccc23)c1]

A mixture of 2,4-dichloroquinazoline (2.05 g, 1.03 mmol), Pd(PPh₃)₄ (103 mg, 0.09 mmol), K₂CO₃ (154 mg, 1.11 mmol) and (3-cyanophenyl)boronic acid (169 mg, 1.15 mmol) in CH₃CN/H₂O (3:1) was heated to 40° C. o/n. The reaction was cooled to rt, diluted with EtOAc, washed with H₂O, dried (MgSO₄), filtered and concentrated. Purification by MPLC (SiO₂, EtOAc/Hexanes, 0-100%) provided the title compound. GC/MS (EI, M+) 264/265.

### Preparation of Intermediate I-49; 3-(2-Chloro-6-methyl-pyrimidin-4-yl)benzonitrile

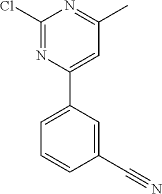 [SMILES: Cc1cc(-c2cccc(C#N)c2)nc(Cl)n1]

The procedure used in the preparation of Intermediate 1-49 was used to prepare the title compound from 2,4-dichloro-6-methyl-pyrimidine and (3-cyanophenyl)boronic acid. GC/MS (EI, M+) 229.

### Preparation of Intermediate I-50; 3-(2-Chloro-5-methyl-pyrimidin-4-yl)benzonitrile

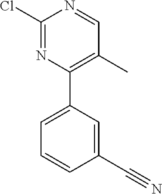 [SMILES: Cc1cnc(Cl)nc1-c1cccc(C#N)c1]

The procedure used in the preparation of Intermediate 1-49 was used to prepare the title compound from 2,4-dichloro-5-methyl-pyrimidine and (3-cyanophenyl)boronic acid. GC/MS (EI, M+) 228.

### Preparation of Intermediate I-51; tert-Butyl N-(3-amino-5-methoxy-phenyl)carbamate

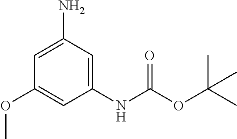 [SMILES: COc1cc(N)cc(NC(=O)OC(C)(C)C)c1]

A solution of 3-amino-5-methoxy-benzoic acid (533 mg, 2.00 mmol) and Et₃N (0.30 mL) in acetone (10 mL) at 0° C. was treated with a solution of ethyl chloroformate (0.21 mL, 2.2 mmol) in acetone (10 mL). The solution was stirred for 0.5 h and a solution of NaN3 (264 mg, 4.06 mmol) in acetone (10 mL) was added and the reaction stirred for 1 h at 0° C. The reaction was extracted with toluene, dried (MgSO₄) and filtered. The resulting solution was heated to reflux for 1 h. Water (20 mL) was added and the reaction refluxed for 1 h. The reaction was cooled to rt and the layers separated. The organics were dried (MgSO₄), filtered and concentrated. Purification by MPLC (SiO₂, EtOAc/Hexanes, 0-100%) provided the title compound. ¹H NMR (CDCl₃) δ 6.76 (t, 1H), 6.66 (t, 1H), 6.63 (s, 1H), 3.71 (s, 3H), 1.50 (s, 9H).

### Preparation of Intermediate I-52; tert-Butyl N-[3-[[4-(3-cyano-4-methoxy-phenyl)pyrimidin-2-yl]amino]-5-methoxy-phenyl]carbamate

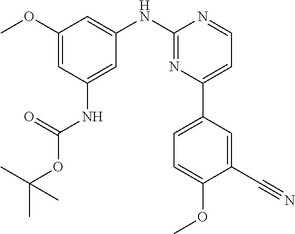 [SMILES: COc1cc(NC(=O)OC(C)(C)C)cc(Nc2nccc(-c3ccc(OC)c(C#N)c3)n2)c1]

The procedure used in the preparation of Intermediate I-11 was used to prepare the title compound from tert-butyl N-(3-amino-5-methoxy-phenyl)carbamate and 5-(2-chloropyrimidin-4-yl)-2-methoxy-benzonitrile. ¹H NMR (DMSO-d₆) δ 9.64 (s, 0.3H), 9.31 (s, 0.7H), 8.65-8.57 (m, 1H), 8.57 (d, 1H), 8.54 (d, 1H), 7.66 (s, 1H), 7.48 (d, 1H), 7.40 (d, 1H), 7.17-7.13 (m, 1H), 6.67 (s, 1H), 4.01 (s, 3H), 3.73 (s, 3H), 1.49 (s, 9H).

### Preparation of Intermediate I-53; 3-(tert-Butoxycarbonylamino)-5-methoxy-benzoic acid

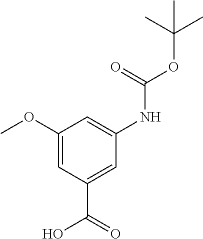 [SMILES: COc1cc(NC(=O)OC(C)(C)C)cc(C(=O)O)c1]

A solution of 3-amino-5-methoxy-benzoic acid (2.062 g, 12.3 mmol) in THF/H₂O (1:1, 24 mL), was treated with NaOH (2.2 N, 6.3 mL, 13.9 mmol) and di-tert-butyl dicarbonate (4.071 g, 18.7 mmol) was stirred at rt o/n. The reaction was acidified with KHSO₄ (sat., aq.) and the resulting solid collected by vacuum filtration to give the title compound. ¹H NMR (DMSO-d₆) δ 9.56 (s, 1H), 7.72 (s, 1H), 7.31 (t, 1H), 7.06 (dd, 1H), 3.76 (s, 3H), 1.48 (s, 9H).

### Preparation of Intermediate I-54; Ethyl 3-(tert-butoxycarbonylamino)-5-methoxy-benzoate

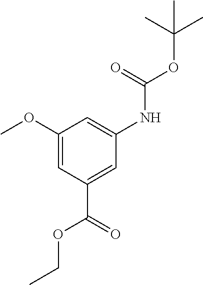 [SMILES: CCOC(=O)c1cc(NC(=O)OC(C)(C)C)cc(OC)c1]

A solution of 3-(tert-butoxycarbonylamino)-5-methoxy-benzoic acid (480 mg, 1.80 mmol) in DMF (2 mL) was treated with Cs₂CO₃ (0.32 g, 0.98 mmol) and ethyl iodide (0.10 mL, 1.25 mmol) and stirred o/n. The reaction was diluted with EtOAc, washed with H₂O and brine, dried (MgSO₄), filtered and concentrated. Purification by MPLC (SiO₂, EtOAc/Hexanes, 0-100%) provided the title compound. ¹H NMR (CDCl₃) δ 7.45-7.37 (m, 2H), 7.26-7.24 (m, 1H), 4.36 (q, 2H), 3.84 (s, 3H), 1.52 (s, 9H), 1.38 (t, 3H).

### Preparation of Intermediate I-55; Ethyl 3-amino-5-methoxy-benzoate

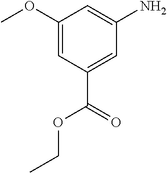

A solution of ethyl 3-(tert-butoxycarbonylamino)-5-methoxy-benzoate in CH₂Cl₂ (10 mL) was treated with TFA (1 mL) and stirred for 1.5 h. The reaction was diluted with EtOAc, quenched with NaHCO₃ (sat., aq.), washed with H₂O and brine, dried (MgSO₄), filtered, and concentrated to provide the title compound. ¹H NMR (CDCl₃) δ 7.10-6.98 (m, 2H), 6.41 (t, 1H), 4.35 (q, 2H), 3.81 (s, 3H), 1.39 (t, 3H).

### Preparation of Intermediate I-56; 2-[(1-Acetyl-4-piperidyl)oxy]-5-[2-[(3-amino-5-methoxy-phenyl)amino]pyrimidin-4-yl]benzonitrile

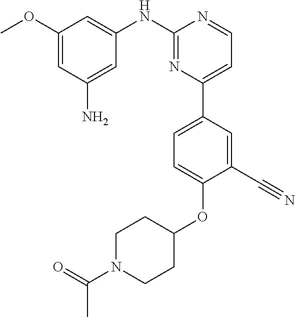 [SMILES: COc1cc(N)cc(Nc2nccc(-c3ccc(OC4CCN(C(C)=O)CC4)c(C#N)c3)n2)c1]

Step 1. The procedure used in the preparation of Intermediate I-11 was used to prepare tert-butyl N-[3-[[4-[4-[(1-acetyl-4-piperidyl)oxy]-3-cyano-phenyl]pyrimidin-2-yl]amino]-5-methoxy-phenyl]carbamate from 2-[(1-acetyl-4-piperidyl)oxy]-5-(2-chloropyrimidin-4-yl)benzonitrile and tert-butyl N-(3-amino-5-methoxy-phenyl)carbamate.

Step 2. A solution of tert-butyl N-[3-[[4-[4-[(1-acetyl-4-piperidyl)oxy]-3-cyano-phenyl]pyrimidin-2-yl]amino]-5-methoxy-phenyl]carbamate was treated with 10% TFA in CH₂Cl₂ for 1 h. The reaction was quenched with NaHCO₃ (sat., aq.), extracted with EtOAc, dried (MgSO₄), filtered, and concentrated to provide the title compound. ¹H NMR (Selected Peaks) (DMSO-d₆) δ 9.38 (s, 1H), 8.56 (d, 1H), 8.52 (d, 1H), 8.46 (dd, 1H), 7.55 (d, 1H), 7.43 (d, 1H), 6.80 (s, 1H), 6.61 (t, 1H), 5.83 (t, 1H), 3.68 (s, 3H), 1.99 (s, 3H).

### Preparation of Intermediate I-57; tert-Butyl N-[3-[[4-(3-cyano-4-methoxy-phenyl)pyrimidin-2-yl]amino]phenyl]carbamate

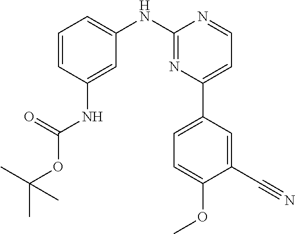 [SMILES: COc1ccc(-c2ccnc(Nc3cccc(NC(=O)OC(C)(C)C)c3)n2)cc1C#N]

The procedure used to prepare Intermediate I-11 was used to prepare the title compound from 5-(2-chloropyrimidin-4-yl)-2-methoxy-benzonitrile and tert-butyl N-(3-aminophenyl)carbamate. ¹H NMR (DMSO-d₆) δ 9.64 (s, 1H), 9.33 (s, 1H), 8.63 (dd, 1H), 8.56 (d, 1H), 8.53 (d, 1H), 8.16 (s, 1H), 7.47 (d, 1H), 7.39 (d, 1H), 7.30 (d, 1H), 7.15 (t, 1H), 6.96 (d, 1H), 4.00 (s, 3H), 1.49 (s, 9H).

### Preparation of Intermediate I-58; 5-(2-Chloropyrimidin-4-yl)-3-methoxy-2-tetrahydropyran-4-yloxy-benzonitrile

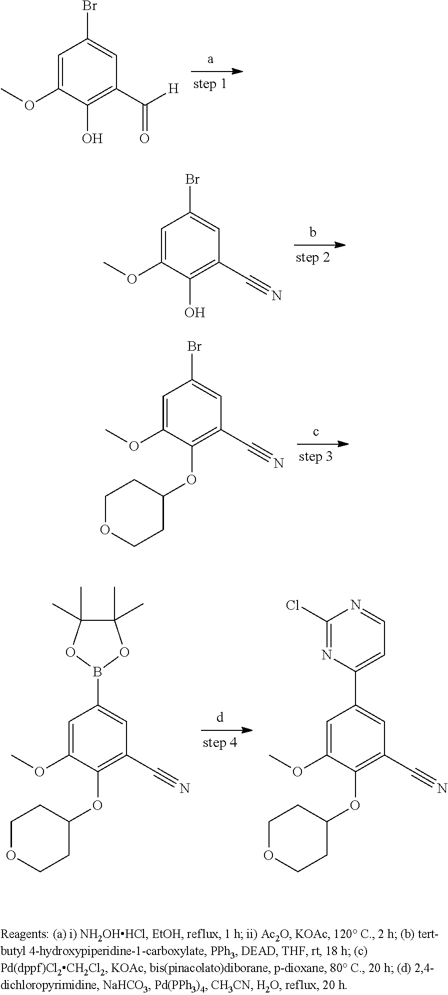

Step 1. 5-Bromo-2-hydroxy-3-methoxy-benzonitrile: A mixture of 5-bromo-2-hydroxy-3-methoxy-benzaldehyde (2.31 g, 10.0 mmol) and hydroxylamine hydrogen chloride (0.834 g, 12.0 mmol) in EtOH (10 mL) was stirred at reflux for 1 h. After removal of EtOH and drying in vacuo, the residue was added to Ac₂O (10 mL) and KOAc (2.0 g) and the solution was stirred at 120° C. for 2 h. After cooling to rt, the reaction mixture was added H₂O (100 mL) and MeOH (10 mL), and basified with solid K₂CO₃ to about pH 10. After stirring for 24 h, the mixture was acidified with concentrated (conc.) HCl (aq) to pH 4.5. The resulting precipitate was collected and dried in vacuo to give 2.1 g of the title compound as an off-white powder.

Step 2. 5-Bromo-3-methoxy-2-tetrahydropyran-4-yloxy-benzonitrile: To a solution of 5-bromo-2-hydroxy-3-methoxy-benzonitrile (1.14 g, 5.0 mmol) in dry THF (20 mL) was added tetrahydropyran-4-ol (0.56 g, 5.5 mmol), PPh₃ (1.57 g, 6.0 mmol), followed by addition of DEAD (1.0 mL, 6.0 mmol) at 0° C. After stirring at rt for 18 h, the reaction mixture was concentrated under reduced pressure. The residue was purified by column chromatography (SiO₂, EtOAc/Hexanes, 0-100%) to afford the title compound (1.45 g, 78.0%).

Step 3. 3-Methoxy-2-tetrahydropyran-4-yloxy-5-(4,4,5,5-tetramethyl-1,3,2-dioxaborolan-2-yl)benzonitrile: To a solution of 5-bromo-3-methoxy-2-tetrahydropyran-4-yloxy-benzonitrile (1.45 g, 4.66 mmol)) in p-dioxane (30 mL) was added Pd(dppf)Cl₂.CH₂Cl₂ (0.204 g, 0.25 mmol), and KOAc (1.47 g, 15 mmol). After stirring at 80° C. for 20 h, the mixture was filtered to remove KOAc, and the filtrate was concentrated under reduced pressure. The residue was purified by column chromatography (SiO₂, EtOAc/Hexanes, 0-100%) to afford the title compound.

Step 4. 5-(2-Chloropyrimidin-4-yl)-3-methoxy-2-tetrahydropyran-4-yloxy-benzonitrile: To a solution of 3-methoxy-2-tetrahydropyran-4-yloxy-5-(4,4,5,5-tetramethyl-1,3,2-dioxaborolan-2-yl)benzonitrile (4.66 mmol) in CH₃CN (30 mL) and H₂O (10 mL) was added Na₂CO₃ (1.26 g, 15 mmol) and Pd(PPh₃)₄ (0.29 g, 0.25 mmol). After refluxing for 20 h, the mixture was concentrated to remove CH₃CN, and the residue was extracted with EtOAc (200 mL). The organic solution was washed with brine (100 mL), dried (MgSO₄), and concentrated under reduced pressure. The residue was purified by column chromatography (SiO₂, EtOAc/Hexanes, 0-85%) to give the title compound (1.2 g, 75.0%). TOF LC-MS [M+H]⁺ 346.1023.

### Preparation of Intermediate 59: tert-Butyl 4-[4-(2-chloropyrimidin-4-yl)-2-cyano-6-methoxy-phenoxy]piperidine-1-carboxylate

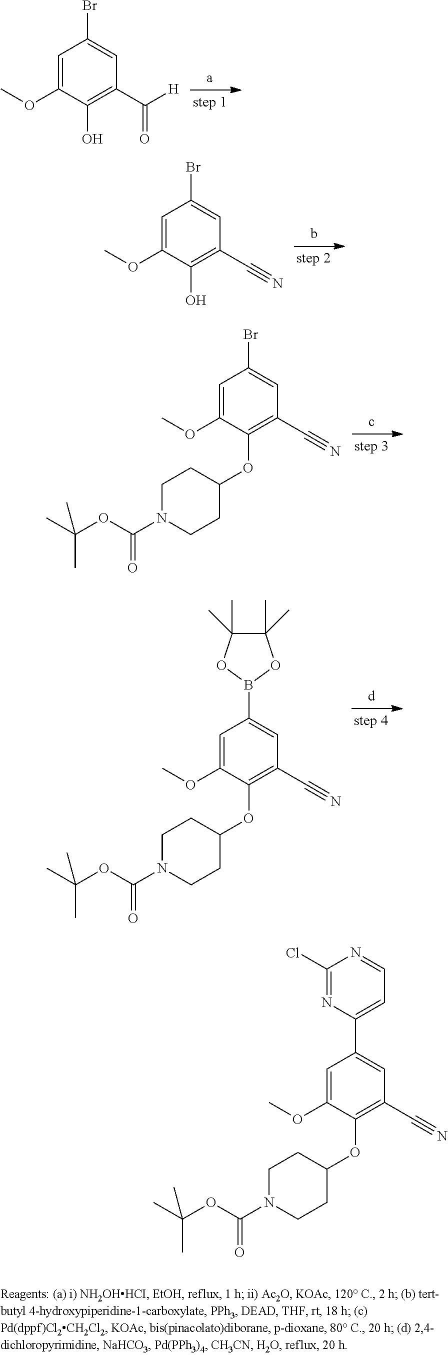 [SMILES: COc1cc(Br)cc(C=O)c1O ; COc1cc(Br)cc(C#N)c1O ; COc1cc(Br)cc(C#N)c1OC1CCN(C(=O)OC(C)(C)C)CC1 ; COc1cc(B2OC(C)(C)C(C)(C)O2)cc(C#N)c1OC1CCN(C(=O)OC(C)(C)C)CC1 ; COc1cc(-c2ccnc(Cl)n2)cc(C#N)c1OC1CCN(C(=O)OC(C)(C)C)CC1]

Step 1. 5-Bromo-2-hydroxy-3-methoxy-benzonitrile: A mixture of 5-bromo-2-hydroxy-3-methoxy-benzaldehyde (2.31 g, 10.0 mmol) and hydroxylamine hydrogen chloride (0.834 g, 12.0 mmol) in EtOH (10 mL) was stirred at reflux for 1 h. Ethanol was removed in vacuo and the residue was treated with Ac₂O (10 mL) and KOAc (2.0 g). The resulting solution was stirred at 120° C. for 2 h. After cooling, the reaction mixture was diluted with H₂O (100 mL) and MeOH (10 mL), and basified with solid K₂CO₃ to ˜pH 10. After standing for 24 h, the mixture was acidified with conc.HCl aqueous solution to ˜pH 4-5. The resulting precipitate was collected and dried in vacuo to give 2.1 g of the title compound as off-white powder.

Step 2. tert-Butyl 4-(4-bromo-2-cyano-6-methoxy-phenoxy)piperidine-1-carboxylate: To a solution of 5-bromo-2-hydroxy-3-methoxy-benzonitrile (1.5 g, 6.6 mmol) in dry THF (40 mL) was added tert-butyl 4-hydroxypiperidine-1-carboxylate (1.40 g, 7.0 mmol), PPh₃ (2.1 g, 8.0 mmol), and DEAD (1.5 mL, 9.5 mmol) at 0° C. After stirring at rt for 18 h, the reaction mixture was concentrated under reduced pressure. The residue was purified by column chromatography (SiO₂, EtOAc/Hexanes, 0-100%) to afford the title compound (2.44 g, 90.0%).

Step 3. tert-Butyl 4-[2-cyano-6-methoxy-4-(4,4,5,5-tetramethyl-1,3,2-dioxaborolan-2-yl)phenoxy]piperidine-1-carboxylate: To a solution of tert-butyl 4-(4-bromo-2-cyano-6-methoxy-phenoxy)piperidine-1-carboxylate (2.46 g, 6.0 mmol) in p-dioxane (25 mL) was added Pd(dppf)Cl₂.CH₂Cl₂ (0.364 g, 0.27 mmol), and KOAc (1.76 g, 18 mmol). After stirring at 80° C. for 20 h, the mixture was filtered to remove KOAc, and the filtrate was concentrated under reduced pressure. The residue was purified by column chromatography (SiO₂, EtOAc/Hexanes, 0-100%) to afford the title compound (2.7 g, 98%).

Step 4. tert-Butyl 4-[4-(2-chloropyrimidin-4-yl)-2-cyano-6-methoxy-phenoxy]piperidine-1-carboxylate: To a solution of tert-butyl 4-[2-cyano-6-methoxy-4-(4,4,5,5-tetramethyl-1,3,2-dioxaborolan-2-yl)phenoxy]piperidine-1-carboxylate (2.7 g, 6.0 mmol) in CH₃CN (20 mL) and H₂O (7 mL) was added Na₂CO₃ (1.25 g, 15 mmol) and Pd(PPh₃)₄ (0.2 g, 0.17 mmol). After refluxing for 20 h, the mixture was concentrated to remove CH₃CN, and the residue was extracted with EtOAc (200 mL). The organic solution was washed with brine (100 mL), dried (MgSO₄) and concentrated under reduced pressure. The residue was purified by column chromatography (SiO₂, EtOAc/Hexanes, 0-85%) to give the title compound (1.6 g, 60.0%).

### Preparation of Intermediate I-60; tert-Butyl 4-[2-cyano-6-methoxy-4-[2-[(4-morpholinophenyl)amino]pyrimidin-4-yl]phenoxy]piperidine-1-carboxylate

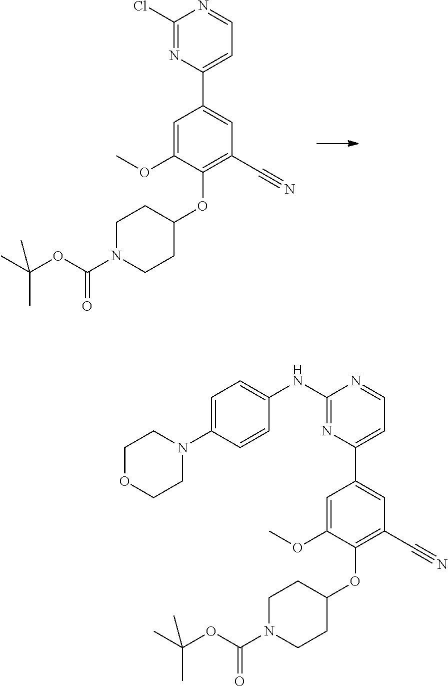 [SMILES: COc1cc(-c2ccnc(Cl)n2)cc(C#N)c1OC1CCN(C(=O)OC(C)(C)C)CC1 ; COc1cc(-c2ccnc(Nc3ccc(N4CCOCC4)cc3)n2)cc(C#N)c1OC1CCN(C(=O)OC(C)(C)C)CC1]

A solution of tert-butyl 4-[4-(2-chloropyrimidin-4-yl)-2-cyano-6-methoxy-phenoxy]piperidine-1-carboxylate (1.60 g, 3.6 mmol) and 4-(morpholin-4-yl)aniline (0.96 g, 5.4 mmol) in EtOH (10 mL) and p-dioxane (10 mL) was stirred at reflux for 48 h. After concentrated under reduce pressure, the residue was purified by column chromatography (SiO₂, EtOAc/Hexanes, 0-100%) to give the title compound; LC-MS [M+H]⁺ 587.

### Preparation of Intermediate I-61; 3-Methoxy-5-[2-[(4-morpholinophenyl)amino]pyrimidin-4-yl]-2-(4-piperidyloxy)benzonitrile

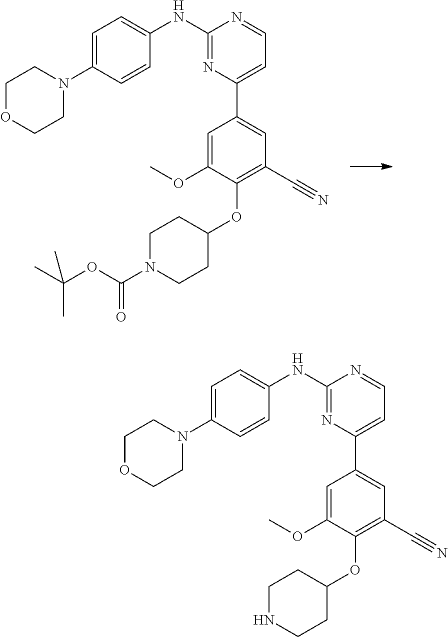 [SMILES: COc1cc(-c2ccnc(Nc3ccc(N4CCOCC4)cc3)n2)cc(C#N)c1OC1CCN(C(=O)OC(C)(C)C)CC1 ; COc1cc(-c2ccnc(Nc3ccc(N4CCOCC4)cc3)n2)cc(C#N)c1OC1CCNCC1]

To a solution of crude tert-butyl 4-[2-cyano-6-methoxy-4-[2-[(4-morpholinophenyl)amino]pyrimidin-4-yl]phenoxy]piperidine-1-carboxylate (3.6 mmol) in CH₂Cl₂ (20 mL) was added TFA (4 mL) at rt. After stirring at rt for 2 h, the reaction mixture was concentrated under reduced pressure, and the residue was taken up in H₂O (50 mL) and basified by K₂CO₃ to form a precipitate which was isolated through filtration and dried in vacuo. For analytical purposes, the crude compound was purified by reverse phase column chromatography (C₁₈, CH₃CN/H₂O with 0.1% TFA, 0-95%) to give the title compound as the corresponding TFA salt. ¹H NMR (DMSO-d₆) δ 9.50 (s, 1H), 8.52 (d, 1H), 8.12-8.09 (m, 2H), 7.65 (d, 2H), 7.46 (d, 1H), 6.93 (d, 2H), 4.55-4.51 (m, 1H), 3.98 (s, 3H), 3.76-3.73 (m, 4H), 3.34 (br s, 1H), 3.05-3.03 (m, 4H), 3.01-2.97 (m, 2H), 2.49-2.44 (m, 2H), 1.89-1.85 (m, 2H), 1.61-1.52 (m, 2H). TOF LC-MS [M+H]⁺ 487.2393.

### Preparation of Intermediate I-62; N-[4-(2-Chloropyrimidin-4-yl)phenyl]-2-methyl-propanamide

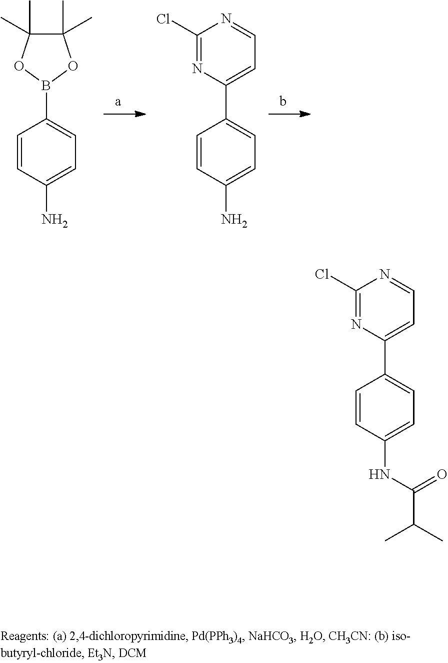

Step 1. 4-(2-chloropyrimidin-4-yl)aniline: To a solution of 4-(4,4,5,5-tetramethyl-1,3,2-dioxaborolan-2-yl)aniline (1.0 g, 4.56 mmol) in CH₃CN (30 mL) and H₂O (10 mL), 2,4-dichloropyrimidine (0.68 g, 4.56 mmol), NaHCO₃ (1.15 g, 13.68 mmol), and Pd(PPh₃)₄ (0.26 g, 0.225 mmol) were added. The resulting mixture was stirred for 16 h at 80° C. The reaction was cooled, diluted with EtOAc, washed with H₂O, and concentrated onto silica. The residue was purified by column chromatography (SiO₂, EtOAc/Hexanes, 0-100%) to afford the title compound (0.53 g, 56%).

Step 2. N-[4-(2-chloropyrimidin-4-yl)phenyl]-2-methyl-propanamid: iso-Butyryl-chloride (0.300 mL, 2.84 mmol) was added to a solution of 4-(2-chloropyrimidin-4-yl)aniline (0.53 g, 2.58 mmol) in DCM (15 mL), followed by portionwise addition of Et₃N (0.900 mL, 6.45 mmol). The resulting mixture was stirred for 30 minutes at rt. The reaction was diluted with DCM and washed with saturated aqueous NaHCO₃ and 1N HCl(aq) solution. The residue was dried in vacuo to afford the title compound (0.77 g, 100%). GC/MS (EI, M+) 300.

### Preparation of Intermediate I-63; 2-(3-Aminophenyl)-N-(2-diethylaminoethyl)-N-ethyl-acetamide

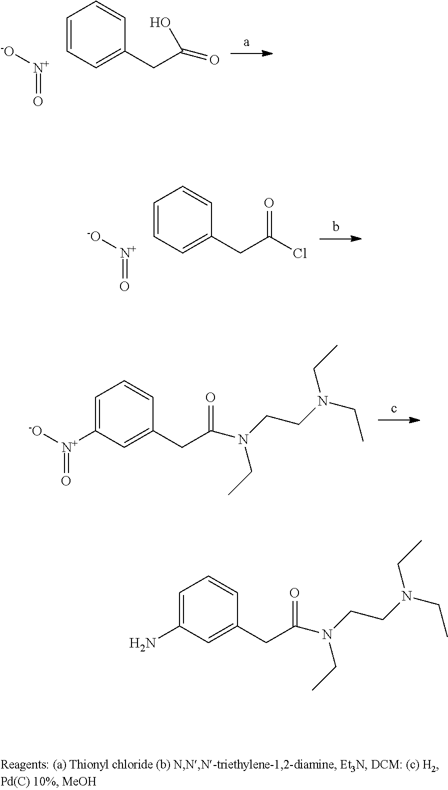

Step 1. 2-(3-nitrophenyl)acetyl chloride: A solution of 2-(3-nitrophenyl)acetic acid (1.0 g, 5.5 mmol) in thionyl chloride (15 mL), was refluxed for 2 hours. The solution was stripped via rotavap and co-stripped with DCM (2×30 mL) to remove residual thionyl chloride, and is used as is in the following step.

Step 2. N-(2-diethylaminoethyl)-N-ethyl-2-(3-nitrophenyl)acetamide: To a solution of 2-(3-nitrophenyl)acetyl chloride (1.38 mmol) in DCM (10 mL), Et₃N (0.600 mL, 4.14 mmol), and N,N′,N′-triethylethane-1,2-diamine (0.298 g, 2.07 mmol) were added. The resulting mixture was stirred for 2 h at rt. The mixture was further diluted with DCM, and washed with H₂O, and dried in vacuo. The material was used as is in the following step.

Step 3. 2-(3-aminophenyl)-N-(2-diethylaminoethyl)-N-ethyl-acetamide: To a solution of N-(2-diethylaminoethyl)-N-ethyl-2-(3-nitrophenyl)acetamide in MeOH (10 mL) was added 20 mg of Pd(C)10% and stirred under an H₂ atmosphere provided via balloon for 18 hours. The solution was filtered through a bed of Celite and concentrated and dried in vacuo to afford the title compound (0.350 g, 92%). GC/MS (EI, M+) 277 parent observed.

### Preparation of Intermediate I-64; 2-[4-[[4-[4-(2-Methylpropanoylamino)phenyl]pyrimidin-2-yl]amino]phenyl]acetic acid

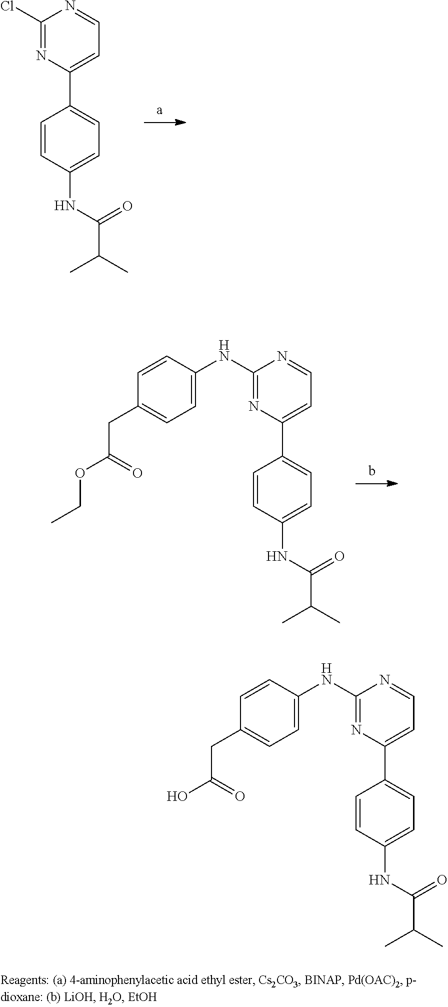 [SMILES: CC(C)C(=O)Nc1ccc(-c2ccnc(Cl)n2)cc1 ; CCOC(=O)Cc1ccc(Nc2nccc(-c3ccc(NC(=O)C(C)C)cc3)n2)cc1 ; CC(C)C(=O)Nc1ccc(-c2ccnc(Nc3ccc(CC(=O)O)cc3)n2)cc1]

Step 1. Ethyl 2-[4-[[4-[4-(2-methylpropanoylamino)phenyl]pyrimidin-2-yl]amino]phenyl]acetate: To a solution of N-[4-(2-chloropyrimidin-4-yl)phenyl]-2-methyl-propanamide (0.525 g, 1.75 mmol) in p-dioxane (30 mL), 4-aminophenyl acetic acid ethyl ester (0.313 g, 1.75 mmol), Cs₂CO₃ (1.14 g, 3.5 mmol), BINAP (0.201 g, 0.324 mmol), and Pd(OAc)₂ (0.067 g, 0.298 mmol) were added. The resulting mixture was stirred for 2 h at 90° C. The mixture was allowed to cool, diluted with EtOAc, and concentrated onto silica. The residue was purified by column chromatography (SiO₂, EtOAc/Hexanes, 0-100%) to afford the title compound (0.48 g, 62%).

Step 2. 2-[4-[[4-[4-(2-methylpropanoylamino)phenyl]pyrimidin-2-yl]amino]phenyl]acetic acid: To a solution of 2-[4-[[4-[4-(2-methylpropanoylamino)phenyl]pyrimidin-2-yl]amino]phenyl]acetate (0.48 g, 1.08 mmol) in EtOH (10 mL), LiOH (4N aqueous solution, 3 mL) was added. The resulting mixture was stirred for 2 h at rt. Ethanol was removed via rotavap and the pH of the resulting aqueous mixture was adjusted to pH 5 by addition of 1N aqueous HCl. The resulting precipitate was collected by filtration, washed with H₂O, and dried in vacuo to afford the title compound (0.44 g, 98%). ¹H NMR (DMSO-d₆) δ 10.28 (s, 1H), 9.74 (s, 1H), 8.60-8.58 (m, 2H), 8.47-8.45 (m, 1H), 7.79-7.72 (m, 3H), 7.54-7.49 (m, 1H), 7.21-7.19 (m, 2H), 3.51 (s, 2H), 2.75-2.71 (m, 1H), 1.16 (d, 6H). LC-MS [M+H]⁺ 416

### Preparation of Intermediate I-65; 4-(Pyrrolidin-1-ylsulfonylmethyl)aniline

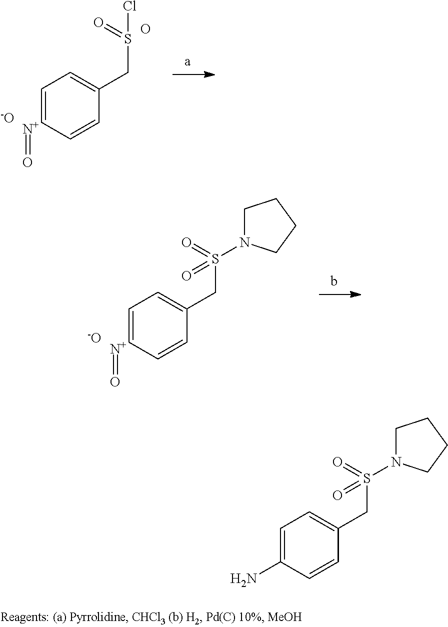

Step 1. 1-[(4-nitrophenyl)methylsulfonyl]pyrrolidine: To a solution of 2-(3-nitrophenyl)acetyl chloride (1.0 mmol) in CHCl₃ (5 mL), pyrrolidine (0.213 g, 3.0 mmol) was added. The resulting mixture was stirred for 4 h at rt. The mixture was concentrated onto silica and the residue was purified by column chromatography (SiO₂, EtOAc/Hexanes, 0-100%) to afford the title compound (0.20 g, 74%).

Step 2. 4-(pyrrolidin-1-ylsulfonylmethyl)aniline: To a solution of 1-[(4-nitrophenyl)methylsulfonyl]pyrrolidine (0.20 g, 0.74 mmol) in MeOH (10 mL) was added 125 mg of Pd(C)10% and stirred under an atmosphere of H₂ gas (g) (balloon) over a period of 4 h. The solution was filtered through a bed of Celite® and concentrated and dried in vacuo to afford the title compound (0.136 g, 77%). LC-MS [M+H]⁺ 241.

### Preparation of Intermediate I-66: 5-[2-[(4-Morpholinophenyl)amino]pyrimidin-4-yl]-2-(pyrrolidin-3-ylmethoxy)benzonitrile

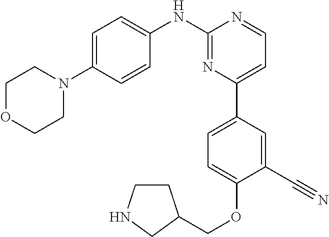 [SMILES: N#Cc1cc(-c2ccnc(Nc3ccc(N4CCOCC4)cc3)n2)ccc1OCC1CCNC1]

This compound was prepared according to the procedure described for the preparation of Intermediate I-5 using tert-butyl 3-(hydroxymethyl)pyrrolidine-1-carboxylate, followed by the procedure of Standard Method E; BOC Deprotection. ¹H NMR (DMSO-d₆) δ 9.51 (s, 1H), 8.84 (br s, 2H, TFA), 8.54-8.47 (m, 3H), 7.65 (d, 2H), 7.45 (d, 1H), 7.41 (d, 1H), 6.96 (d, 2H), 4.34-4.21 (m, 2H), 3.77-3.74 (m, 4H), 3.46-3.39 (m, 1H), 3.35-3.21 (m, 2H), 3.09-3.03 (m, 5H), 2.86-2.79 (m, 1H), 2.19-2.10 (m, 1H), 1.85-1.76 (m, 1H). TOF LC-MS [M+H]⁺ 457.2367.

### Preparation of Intermediate I-67: 5-[2-[(4-Morpholinophenyl)amino]pyrimidin-4-yl]-2-[2-(4-piperidyl)ethoxy]benzonitrile

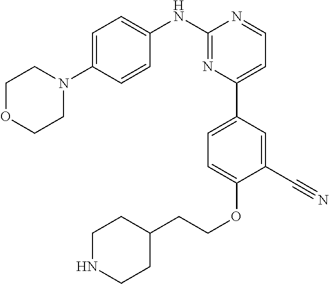 [SMILES: N#Cc1cc(-c2ccnc(Nc3ccc(N4CCOCC4)cc3)n2)ccc1OCCC1CCNCC1]

This compound was prepared according to the procedure described for the preparation of Intermediate I-5 using tert-butyl 4-(2-hydroxyethyl)piperidine-1-carboxylate, followed by the procedure of Standard Method E; BOC Deprotection. ¹H NMR (DMSO-d₆) δ 9.52 (s, 1H), 8.60 (br s, 1H, TFA), 8.52-8.45 (m, 3H), 8.31 (br s, 1H, TFA), 7.66 (m, 2H), 7.45 (d, 1H), 7.41 (d, 1H), 6.98 (d, 2H), 4.30 (t, 2H), 3.78-3.75 (m, 4H), 3.29 (apparent d, 2H), 3.11-3.08 (m, 4H), 2.93-2.84 (m, 2H), 1.92 (apparent d, 2H), 1.83-1.75 (m, 3H), 1.43-1.35 (m, 2H). TOF LC-MS [M+H]⁺ 485.2762.

### Preparation of Intermediate I-68: tert-Butyl 3-[2-cyano-4-[2-[(4-morpholinophenyl)amino]pyrimidin-4-yl]phenoxy]azetidine-1-carboxylate

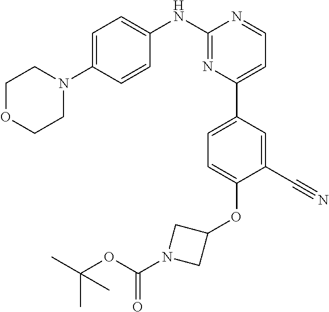 [SMILES: CC(C)(C)OC(=O)N1CC(Oc2ccc(-c3ccnc(Nc4ccc(N5CCOCC5)cc4)n3)cc2C#N)C1]

This compound was prepared according to the procedure described for the preparation of Intermediate I-5 using tert-butyl 3-hydroxyazetidine-1-carboxylate. ¹H NMR (DMSO-d₆) δ 9.48 (s, 1H), 8.55 (d, 1H), 8.50 (d, 1H), 8.44-8.41 (m, 1H), 7.64-7.62 (m, 2H), 7.40 (d, 1H), 7.16 (d, 1H), 6.94-6.91 (m, 2H), 5.27-5.21 (m, 1H), 4.43-4.36 (m, 2H), 3.93-3.87 (m, 2H), 3.76-3.73 (m, 4H), 3.06-3.03 (m, 4H), 1.40 (s, 9H). TOF LC-MS [M+H]⁺ 529.2522.

### Preparation of Intermediate I-69: 2-(Azetidin-3-yloxy)-5-[2-[(4-morpholinophenyl)amino]pyrimidin-4-yl]benzonitrile

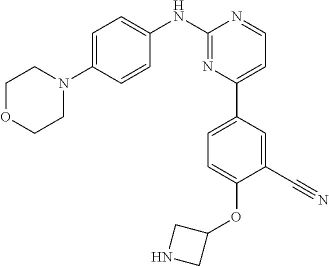 [SMILES: N#Cc1cc(-c2ccnc(Nc3ccc(N4CCOCC4)cc3)n2)ccc1OC1CNC1]

This compound was prepared from tert-Butyl 3-[2-cyano-4-[2-[(4-morpholinophenyl)amino]pyrimidin-4-yl]phenoxy]azetidine-1-carboxylate using the procedure of Standard Method E; BOC Deprotection. ¹H NMR (DMSO-d₆) δ 9.46 (s, 1H), 8.53 (d, 1H), 8.48 (d, 1H), 8.42-8.39 (m, 1H), 7.65-7.62 (m, 2H), 7.37 (d, 1H), 7.13 (d, 1H), 6.92 (d, 2H), 5.27-5.20 (m, 1H), 3.88-3.83 (m, 2H), 3.76-3.73 (m, 4H), 3.60-3.55 (m, 2H), 3.34 (br s, 1H), 3.06-3.03 (m, 4H). TOF LC-MS [M+H]⁺ 429.1945.

### Preparation of Intermediate I-70; 5-{2-[(3-Amino-5-methoxyphenyl)amino]pyrimidin-4-yl}-2-methoxybenzonitrile

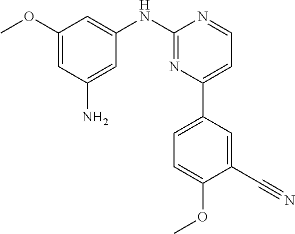 [SMILES: COc1cc(N)cc(Nc2nccc(-c3ccc(OC)c(C#N)c3)n2)c1]

This compound was prepared from tert-butyl N-[3-[[4-(3-cyano-4-methoxy-phenyl)pyrimidin-2-yl]amino]-5-methoxy-phenyl]carbamate using the procedure of Standard Method E; BOC Deprotection. ¹H NMR (DMSO-d₆) δ 9.41 (s, 1H), 8.56 (d, 1H), 8.54-8.46 (m, 2H), 7.44 (d, 1H), 7.43 (d, 1H), 6.81 (s, 1H), 6.62 (t, 1H), 5.83 (t, 1H), 5.07 (br s, 2H), 4.01 (s, 3H), 3.68 (s, 3H). TOF LC-MS [M+H]⁺ 348.1449.

### Preparation of Intermediate I-71; 3-{[4-(3-Cyano-4-methoxyphenyl)pyrimidin-2-yl]amino}-5-methoxybenzoic acid

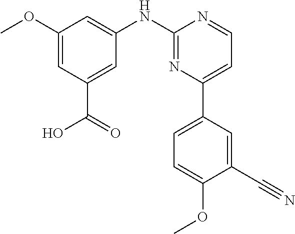 [SMILES: COc1cc(Nc2nccc(-c3ccc(OC)c(C#N)c3)n2)cc(C(=O)O)c1]

Standard Method G, Ester Hydrolysis was used to prepare the title compound from ethyl 3-[[4-(3-cyano-4-methoxy-phenyl)pyrimidin-2-yl]amino]-5-methoxy-benzoate. ¹H NMR (DMSO-d₆) δ 9.61 (s, 1H), 8.62-8.50 (m, 4H), 7.47 (d, 1H), 7.45-7.36 (m, 2H), 7.11 (t, 1H), 6.80 (s, 1H), 6.18 (t, 1H), 4.77 (d, 1H) 4.02 (s, 3H), 3.73 (s, 3H), 3.70-3.64 (m, 1H), 3.20-3.11 (m, 1H), 3.00-2.90 (m, 1H), 1.06 (d, 1H). TOF LC-MS [M+H]⁺ 449.1937.

### Preparation of Intermediate I-72; 3-[2-Cyano-4-[2-[(4-morpholinophenyl)amino]pyrimidin-4-yl]phenoxy]-2,2-dimethyl-propanoic acid

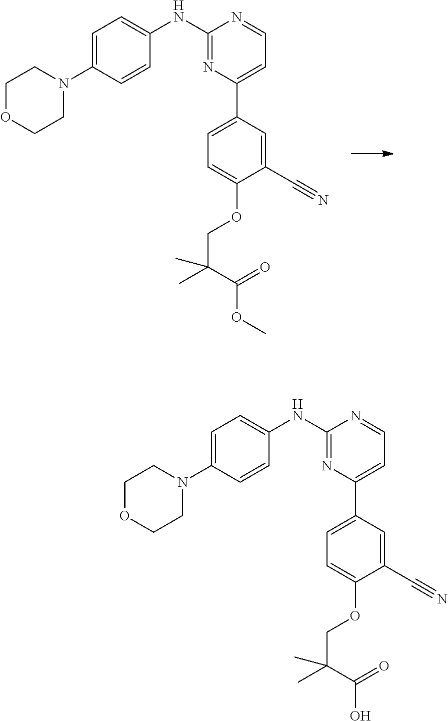 [SMILES: COC(=O)C(C)(C)COc1ccc(-c2ccnc(Nc3ccc(N4CCOCC4)cc3)n2)cc1C#N ; CC(C)(COc1ccc(-c2ccnc(Nc3ccc(N4CCOCC4)cc3)n2)cc1C#N)C(=O)O]

Standard Method G, Ester Hydrolysis was used to prepare the title compound from methyl 3-[2-cyano-4-[2-[(4-morpholinophenyl)amino]pyrimidin-4-yl]phenoxy]-2,2-dimethyl-propanoate. ¹H NMR (DMSO-d₆) δ 9.54 (s, 1H), 8.52-8.45 (m, 3H), 7.66 (d, 2H), 7.46 (d, 1H), 7.41 (d, 1H), 7.00 (apparent d, 2H), 4.23 (s, 2H), 3.78 3.75 (m, 4H), 3.11 (br s, 4H), 1.28 (s, 6H). TOF LC-MS [M+H]⁺ 474.1972.

The structures and physicochemical characterization of synthesized intermediates are provided in Table 1 below. The intermediates were synthesized using the methods outlined above using commercially available starting materials that are well known in the art.

TABLE 1
Additional Intermediates

| No. | Structure | NMR¹H NMR | Method |
|---|---|---|---|
| I-73 | 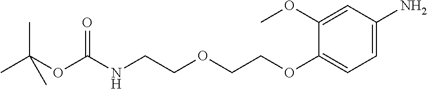 [SMILES: COc1cc(N)ccc1OCCOCCNC(=O)OC(C)(C)C] | ¹H NMR (CDCl₃) δ 6.77 (d, 1H), 6.30 (d, 1H), 6.21 (dd, 1H), 5.13 (br s, 1H), 4.10- 4.05 (m, 2H), 3.82 (s, 3H), 3.80-3.75 (m, 2H), 3.62-3.56 (m, 2H), 3.38-3.30 (m, 2H), 1.44 (s, 9H). | Method A |
| I-74 | 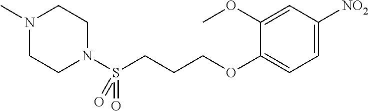 [SMILES: COc1cc([N+](=O)[O-])ccc1OCCCS(=O)(=O)N1CCN(C)CC1] | ¹H NMR (CDCl₃) δ 7.90 (dd, 1H), 7.75 (d, 1H), 7.91 (d, 1H), 4.25 (t, 2H), 3.94 (s, 3H), 3.37-3.30 (m, 4H), 3.19-3.12 (m, 2H), 2.54-2.46 (m, 4H), 2.45-2.35 (m, 2H), 2.33 (s, 3H). | Method B |
| I-75 | 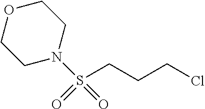 [SMILES: O=S(=O)(CCCCl)N1CCOCC1] | ¹H NMR (CDCl₃) δ 3.81-3.76 (m, 4H), 3.73-3.68 (m, 2H), 3.33-3.26 (m, 4H), 3.14-3.06 (m, 2H), 2.38-2.26 (m, 2H). | I-17 |
| I-76 | 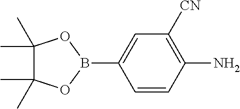 [SMILES: CC1(C)OB(c2ccc(N)c(C#N)c2)OC1(C)C] | ¹H NMR (CDCl₃) δ 7.85 (s, 1H), 7.73 (dd, 1H), 6.71 (d, 2H), 4.60 (bs, 2H), 1.33 (bs, 12H); GC/MS (EI, M+) 244 | I-1 Step 1 |
| I-77 | 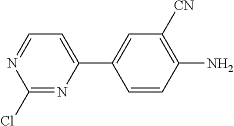 [SMILES: N#Cc1cc(-c2ccnc(Cl)n2)ccc1N] | LC-MS[M + H]⁺230.8 | I-1 Step 2 |
| I-78 | 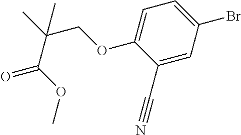 [SMILES: COC(=O)C(C)(C)COc1ccc(Br)cc1C#N] | ¹H NMR (CDCl₃) δ 7.65-7.60 (m, 2H), 6.88 (d, 1H), 4.06 (s, 2H), 3.73 (s, 3H), 1.37 (s, 6H). | I-5 Step 1 |
| I-79 | 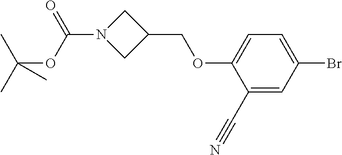 [SMILES: CC(C)(C)OC(=O)N1CC(COc2ccc(Br)cc2C#N)C1] | ¹H NMR (CDCl₃) δ 7.68-7.62 (m, 2H), 6.88 (d, 1H), 4.20 (d, 2H), 4.13 (t, 2H), 3.82-3.75 (m, 2H), 3.08-3.00 (m, 1H), 1.45 (s, 9H). | I-5 Step 1 |
| I-80 | 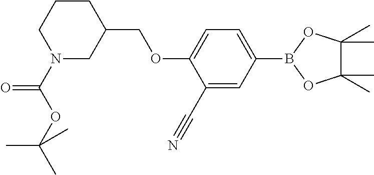 [SMILES: CC(C)(C)OC(=O)N1CCCC(COc2ccc(B3OC(C)(C)C(C)(C)O3)cc2C#N)C1] | ¹H NMR (CDCl₃) δ 8.01 (d, 1H), 7.94-7.91 (m, 1H), 6.92 (d, 1H), 4.03-3.82 (m, 5H), 2.97 (br s, 2H), 2.10-1.85 (m, 2H), 2.04- 1.44 (m, 2H), 1.44 (s, 9H), 2.34 (s, 12H). | I-5 Step 1 |
| I-81 | 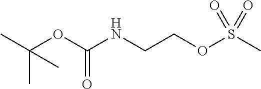 | ¹H NMR (CDCl₃) δ 4.92 (br s, 1H), 4.29 (t, 2H), 3.48 (q, 2H), 3.04 (s, 3H), 1.45 (s, 9H). | Method C |
| I-82 | 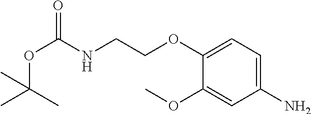 [SMILES: COc1cc(N)ccc1OCCNC(=O)OC(C)(C)C] | ¹H NMR (CDCl₃) δ 6.76 (d, 1H), 6.35 (d, 1H), 6.27 (dd, 1H), 3.99 (t, 2H), 3.83 (s, 3H), 3.52-3.42 (m, 2H), 1.45 (s, 9H). | Method A |
| I-83 | 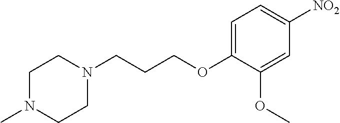 [SMILES: COc1cc([N+](=O)[O-])ccc1OCCCN1CCN(C)CC1] | ¹H NMR (CDCl₃) δ 7.90 (dd, 1H), 7.74 (d, 1H), 6.95 (d, 1H), 4.19 (t, 2H), 3.95 (s, 3H), 2.67-2.20 (m, 10H), 2.28 (s, 3H), 2.06 (quint, 2H). | Method B |
| I-84 | 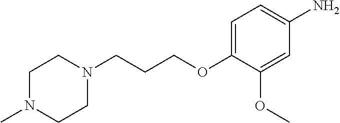 [SMILES: COc1cc(N)ccc1OCCCN1CCN(C)CC1] | ¹H NMR (CDCl₃) δ 6.74 (d, 1H), 6.30 (d, 1H), 6.21 (dd, 1H), 3.98 (t, 2H), 3.81 (s, 3H), 3.50-3.40 (m, 2H), 2.60-2.32 (m, 8H), 2.29 (s, 3H), 2.02-1.92 (m, 2H). | Method A |
| I-85 | 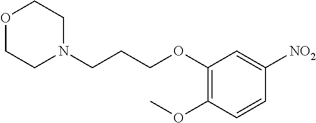 [SMILES: COc1ccc([N+](=O)[O-])cc1OCCCN1CCOCC1] | ¹H NMR (CDCl₃) δ 7.91 (dd, 1H), 7.78 (d, 1H), 6.91 (d, 1H), 4.17 (t, 2H), 3.97 (s, 3H), 3.77-3.70 (m, 4H), 2.54 (t, 2H), 2.52- 2.42 (m, 4H), 2.06 (quint., 2H). | Method B |
| I-86 | 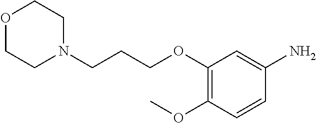 [SMILES: COc1ccc(N)cc1OCCCN1CCOCC1] | ¹H NMR (CDCl₃) δ 6.71 (d, 1H), 6.34 (d, 1H), 6.24 (dd, 1H), 4.03 (t, 2H), 3.79 (s, 3H), 3.76-3.68 (m, 4H), 2.53 (t, 2H), 2.52- 2.43 (m, 4H), 2.02 (quint., 2H). | Method A |
| I-87 | 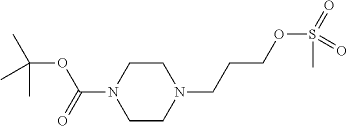 [SMILES: CC(C)(C)OC(=O)N1CCN(CCCOS(C)(=O)=O)CC1] | ¹H NMR (CDCl₃) δ 4.33 (t, 2H), 3.50-3.40 (m, 4H), 3.03 (s, 3H), 2.55-2.46 (m, 2H), 2.46-2.36 (m, 4H), 2.02-1.98 (m, 2H), 1.46 (s, 9H). | Method C |
| I-88 | 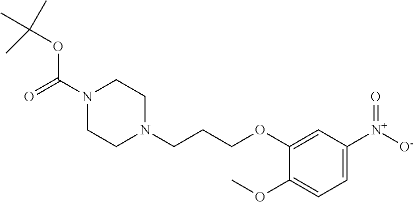 [SMILES: COc1ccc([N+](=O)[O-])cc1OCCCN1CCN(C(=O)OC(C)(C)C)CC1] | ¹H NMR (CDCl₃) δ 7.91 (dd, 1H), 7.77 (d, 1H), 6.91 (d, 1H), 4.17 (t, 2H), 3.96 (s, 3H), 3.48-3.40 (m, 4H), 2.55 (t, 2H), 2.45- 2.36 (m, 4H), 2.10-2.00 (m, 2H), 1.46 (s, 9H). | Method B |
| I-89 | 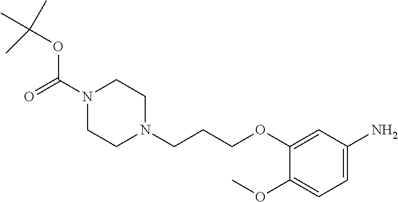 [SMILES: COc1ccc(N)cc1OCCCN1CCN(C(=O)OC(C)(C)C)CC1] | ¹H NMR (CDCl₃) δ 6.71 (d, 1H), 6.34 (d, 1H), 6.24 (dd, 1H), 4.03 (t, 2H), 3.79 (s, 3H), 3.48-3.38 (m, 6H), 2.53 (t, 2H), 2.25- 2.35 (m, 4H), 2.06-1.96 (m, 2H), 1.46 (s, 9H). | Method A |
| I-90 | 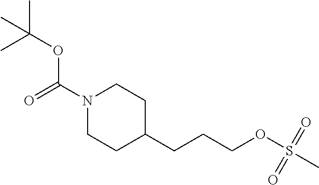 [SMILES: CC(C)(C)OC(=O)N1CCC(CCCOS(C)(=O)=O)CC1] | ¹H NMR (CDCl₃) δ 4.15-4.03 (m, 2H), 3.01 (s, 3H), 2.72-2.60 (m, 2H), 1.82-1.74 (m, 2H), 1.72-1.55 (m, 3H), 1.46 (s, 9H), 1.44-1.27 (m, 4H), 1.18-0.94 (m, 2H). | Method C |
| I-91 | 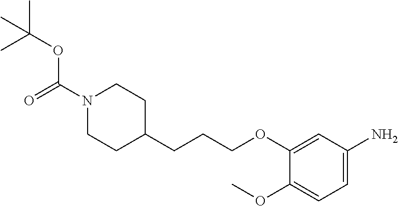 [SMILES: COc1ccc(N)cc1OCCCC1CCN(C(=O)OC(C)(C)C)CC1] | ¹H NMR (CDCl₃) δ 6.71 (d, 1H), 6.31 (d, 1H), 6.23 (dd, 1H), 3.95 (t, 2H), 3.79 (s, 3H), 3.43 (s, 2H), 2.67 (br s, 2H), 1.90- 1.80 (m, 2H), 1.72-1.65 (m, 2H), 1.46 (s, 9H), 1.44-1.35 (m, 3H), 1.18-0.92 (m, 2H). | Method A |
| I-92 | 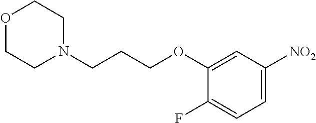 [SMILES: O=[N+]([O-])c1ccc(F)c(OCCCN2CCOCC2)c1] | ¹H NMR (CDCl₃) δ 7.91-7.83 (m, 2H), 7.20 (dd, 1H), 4.20 (t, 2H), 3.78-3.68 (m, 4H), 2.55 (t, 2H), 2.51-2.42 (m, 4H), 2.05 (quint., 2H). | Method B |
| I-93 | 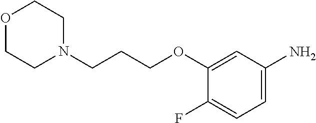 [SMILES: Nc1ccc(F)c(OCCCN2CCOCC2)c1] | ¹H NMR (CDCl₃) δ 6.85 (dd, 1H), 6.32 (dd, 1H), 6.20-6.14 (m, 1H), 4.04 (t, 2H), 3.78-3.70 (m, 6H), 2.64-2.42 (m, 6H), 2.10-1.96 (m, 2H). | Method A |
| I-94 | 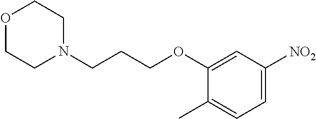 [SMILES: Cc1ccc([N+](=O)[O-])cc1OCCCN1CCOCC1] | ¹H NMR (CDCl₃) δ 7.76 (dd, 1H), 7.66 (d, 1H), 7.25 (br s, 1H), 4.12 (t, 2H), 3.74 (t, 4H), 2.56 (t, 2H), 2.48 (br s, 4H), 2.30 (s, 3H), 2.10-2.00 (m, 2H). | Method B |
| I-95 | 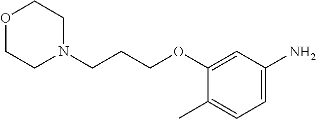 [SMILES: Cc1ccc(N)cc1OCCCN1CCOCC1] | ¹H NMR (CDCl₃) δ 6.89 (d, 1H), 6.24- 6.17 (m, 2H), 3.96 (t, 2H), 3.73 (t, 4H), 3.54 (br s, 2H), 2.58-2.50 (m, 2H), 2.47 (br s, 4H), 2.10 (t, 3H), 2.02-1.92 (m, 2H). | Method A |
| I-97 | 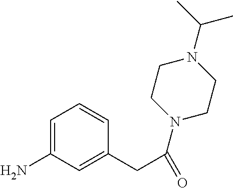 [SMILES: CC(C)N1CCN(C(=O)Cc2cccc(N)c2)CC1] | GC/MS (EI, M+) 261 | Method H |
| I-98 | 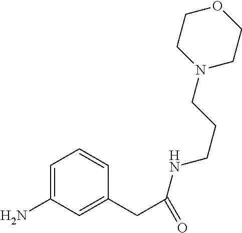 [SMILES: Nc1cccc(CC(=O)NCCCN2CCOCC2)c1] | GC/MS (EI, M+) 277 | Method H |
| I-99 | 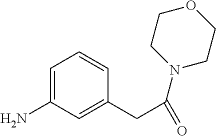 [SMILES: Nc1cccc(CC(=O)N2CCOCC2)c1] | GC/MS (EI, M+) 220 | Method H |
| I-100 | 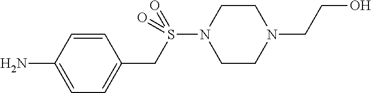 [SMILES: Nc1ccc(CS(=O)(=O)N2CCN(CCO)CC2)cc1] | ¹H NMR (CDCl₃) δ 7.17-7.11 (m, 2H), 6.68-6.65 (m, 2H), 4.12-4.07 (m, 4H), 3.62-3.59 (m, 2H), 3.16-3.14 (m, 4H), 2.54-2.51 (m, 2H), 2.49-2.46 (m, 4H); LC-MS [M + H]⁺ 300 | I-17 |
| I-101 | 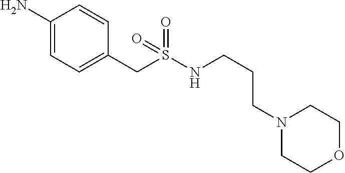 [SMILES: Nc1ccc(CS(=O)(=O)NCCCN2CCOCC2)cc1] | ¹H NMR (CDCl₃) δ 7.20-7.17 (m, 2H), 6.67-6.64 (m, 2H), 4.13 (s, 2H), 3.52- 3.47 (m, 4H), 3.13-3.10 (m, 2H), 2.43- 2.33 (m, 6H), 1.69-1.63 (m, 2H); LC- MS [M + H]⁺ 314 | I-20 |
| I-102 | 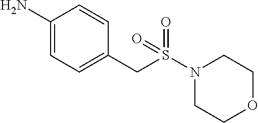 [SMILES: Nc1ccc(CS(=O)(=O)N2CCOCC2)cc1] | ¹H NMR (CDCl₃) δ 7.20-7.17 (m, 2H), 6.67-6.64 (m, 2H), 4.13 (s, 2H), 3.52- 3.47 (m, 4H), 3.13-3.10 (m, 2H), 2.43- 2.33 (m, 6H), 1.69-1.63 (m, 2H); GC/MS (EI, M+) 256 | I-20 |
| I-103 | 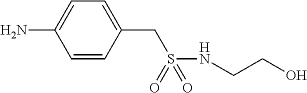 [SMILES: Nc1ccc(CS(=O)(=O)NCCO)cc1] | GC/MS (EI, M+) 230 | I-20 |
| I-104 | 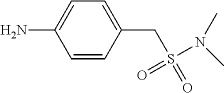 | GC/MS (EI, M+) 214 | I-20 |
| I-105 | 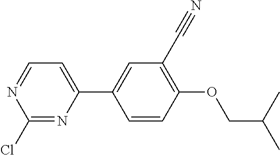 [SMILES: CC(C)COc1ccc(-c2ccnc(Cl)n2)cc1C#N] | TOF LC-MS [M + H]⁺ 288.0817 | I-4 |
| I-106 | 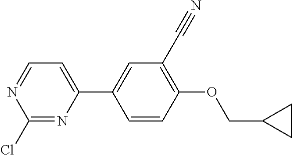 [SMILES: N#Cc1cc(-c2ccnc(Cl)n2)ccc1OCC1CC1] | TOF LC-MS [M + H]⁺ 286.0813 | I-4 |
| I-107 | 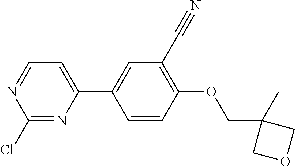 [SMILES: CC1(COc2ccc(-c3ccnc(Cl)n3)cc2C#N)COC1] | TOF LC-MS [M + H]⁺ 316.0800 | I-4 |
| I-108 | 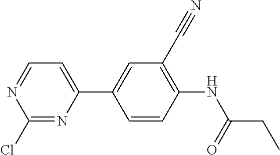 [SMILES: CCC(=O)Nc1ccc(-c2ccnc(Cl)n2)cc1C#N] | TOF LC-MS [M + H]⁺ 287.0706 | I-63 |

### Preparation of Specific Example Compounds

### Example Compound 1

### N-[2-Cyano-4-(2-{[4-(morpholin-4-yl)phenyl]amino}pyrimidin-4-yl)phenyl]-3-methylbutanamide

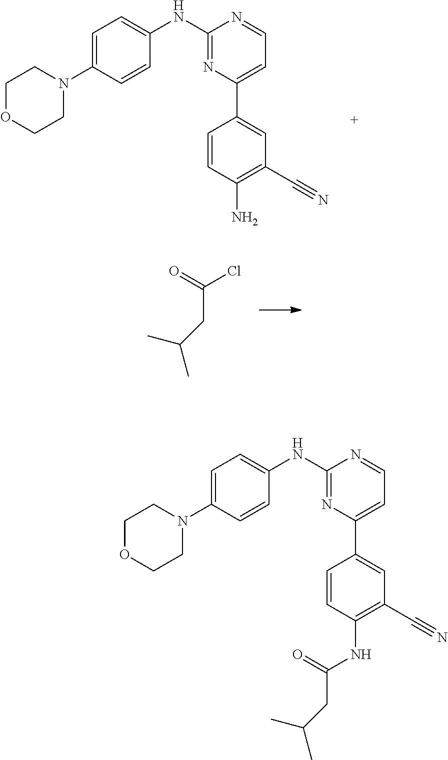

A solution of 2-amino-5-(2-{[4-(morpholin-4-yl)phenyl]amino}pyrimidin-4-yl)benzonitrile (0.10 g, 0.27 mmol) in pyridine (2 mL) was treated with 3-methylbutanoyl chloride (0.080 mL, 0.67 mmol). The resulting mixture was stirred for 3 h at 85° C. in a sealed vial. The residue was concentrated onto SiO₂ and purified by column chromatography on SiO₂ (MeOH/CH₂Cl₂) to afford the title compound (0.03 g, 24%). ¹H NMR (CDCl₃) δ 8.45 (d, 1H), 8.44 (d, 1H), 8.33 (d, 1H), 8.25 (dd, 1H) 7.59-7.57 (m, 2H), 7.07 (d, 1H), 6.99-6.96 (m, 2H), 3.91-3.86 (m, 4H), 3.18-3.14 (m, 4H), 2.37 (d, 2H), 2.25-2.21 (m, 1H), 1.066 (t, 6H). LC-MS [M+H]⁺ 457.23222.

### Example Compound 2

### 4-({4-[3-Cyano-4-(tetrahydro-2H-pyran-4-yloxy)phenyl]pyrimidin-2-yl}amino)-N-[2-(dimethylamino)ethyl]-2-methoxybenzamide

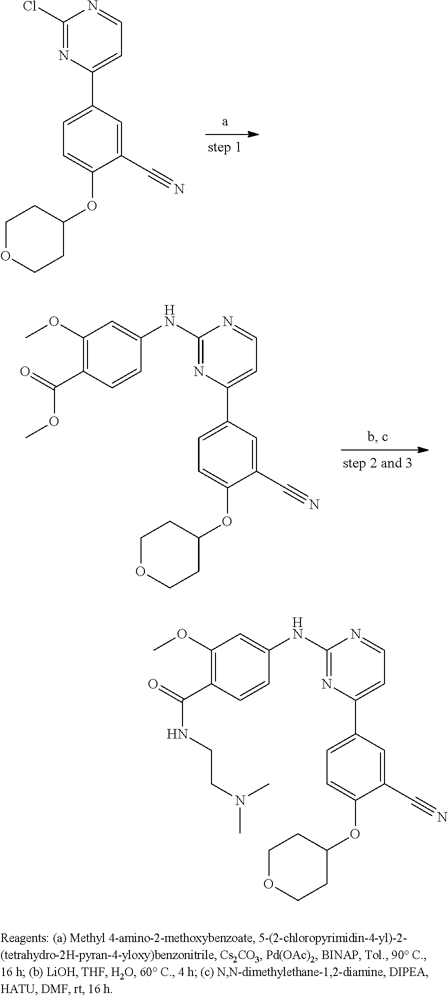 [SMILES: N#Cc1cc(-c2ccnc(Cl)n2)ccc1OC1CCOCC1 ; COC(=O)c1ccc(Nc2nccc(-c3ccc(OC4CCOCC4)c(C#N)c3)n2)cc1OC ; COc1cc(Nc2nccc(-c3ccc(OC4CCOCC4)c(C#N)c3)n2)ccc1C(=O)NCCN(C)C]

Step 1. Methyl 4-({4-[3-cyano-4-(tetrahydro-2H-pyran-4-yloxy)phenyl]pyrimidin-2-yl}amino)-2-methoxybenzoate: Methyl 4-amino-2-methoxybenzoate (1.72 g, 9.49 mmol) and 5-(2-chloropyrimidin-4-yl)-2-(tetrahydro-2H-pyran-4-yloxy)benzonitrile (2.0 g, 6.33 mmol) were added to a flask. Cesium carbonate (6.18 g, 19.0 mmol) and toluene (60.0 mL) were added and the reaction flask was flushed with nitrogen. Palladium acetate (0.21 g, 0.95 mmol) and BINAP (1.0 g, 1.58 mmol) were added and the reaction flask was flushed with nitrogen. The reaction mixture was placed in an oil bath at 90° C. and stirred for 16 h. The reaction was cooled to rt, H₂O (25 mL) and EtOAc (50 mL) were added, and the resulting precipitate was filtered, washed with minimal amounts of H₂O, and EtOAc to afford solid. The filtrates were combined, concentrated in vacuo, and the residue recrystallized/precipitated from EtOAc to provide additional product. The two solids were combined to provide the title compound (2.1 g, 72%). ¹H NMR (DMSO-d₆) δ 10.1 (br s, 1H), 8.62 (d, 2H), 8.48 (s, 1H), 7.90 (s, 1H), 7.72 (d, 1H), 7.59 (t, 2H), 7.39 (d, 1H), 4.98-4.96 (m, 1H), 3.90-3.86 (m, 2H), 3.88 (s, 3H), 3.76 (s, 3H), 3.59-3.56 (m, 2H), 2.08-2.03 (m, 2H), 1.69 (m, 2H); TOF [M+H]⁺ 461.1816.

Step 2. 4-({4-[3-Cyano-4-(tetrahydro-2H-pyran-4-yloxy)phenyl]pyrimidin-2-yl}amino)-2-methoxybenzoic acid: A mixture of methyl 4-({4-[3-cyano-4-(tetrahydro-2H-pyran-4-yloxy)phenyl]pyrimidin-2-yl}amino)-2-methoxybenzoate (1.3 g, 2.83 mmol) and LiOH (0.34 g, 14.1 mmol) in THF/H₂O (2:1, 50 mL) was stirred at 65° C. for 16 h. The reaction mixture was concentrated to 20 mL under reduced pressure and acidified with 1N HCl(aq). The resulting precipitate was filtered and washed with H₂O and dried under reduced pressure to afford the title compound (1.28 g, quant.). ¹H NMR (DMSO-d₆) δ 12.0 (br s, 1H), 10.0 (s, 1H), 8.61 (dd, 2H), 8.48 (d, 1H), 7.89 (s, 1H), 7.71 (d, 1H), 7.60-7.56 (m, 2H), 7.36 (dd, 1H), 4.96 (m, 1H), 3.89-3.85 (m, 2H), 3.87 (s, 3H), 3.58-3.53 (m, 2H), 2.07-2.04 (m, 2H), 1.71-1.66 (m, 2H); LC-MS [M+H]⁺ 447.

Step 3. 4-({4-[3-Cyano-4-(tetrahydro-2H-pyran-4-yloxy)phenyl]pyrimidin-2-yl}amino)-N-[2-(dimethylamino)ethyl]-2-methoxybenzamide: To a mixture of 4-({4-[3-cyano-4-(tetrahydro-2H-pyran-4-yloxy)phenyl]pyrimidin-2-yl}amino)-2-methoxybenzoic acid (0.040 g, 0.09 mmol), N,N-dimethylethane-1,2-diamine (0.015 g, 0.11 mmol) and DIPEA (0.020 mL, 0.11 mmol) in DMF (1 mL) was added HATU (0.043 g, 0.11 mmol). The reaction mixture was stirred for 16 h and purified by reverse phase chromatography (C₁₈, CH₃CN 95% in H₂O with 0.1% TFA). The desired fractions were collected and the solvent evaporated under reduced pressure. The resulting solid was recrystallized from EtOAc/Hexanes to afford the title compound as the trifluoroacetate salt (0.12 g, 21%). ¹H NMR (DMSO-d₆) δ 10.1 (br s, 1H), 9.30 (s, 1H), 8.65-8.61 (m, 2H), 8.47 (dd, 1H), 8.41 (t, 1H), 8.00 (s, 1H), 7.88 (d, 1H), 7.60-7.56 (m, 2H), 7.38 (d, 1H), 4.98-4.94 (m, 1H), 4.00 (s, 3H), 3.90-3.85 (m, 2H), 3.67-3.62 (m, 2H), 3.60-3.54 (m, 2H), 3.29-3.24 (m, 2H), 2.85 (s, 6H), 2.08-2.01 (m, 2H), 1.73-1.66 (m, 2H); TOF [M+H]⁺ 531.2715.

### Example Compound 3

### 4-({4-[3-Cyano-4-(tetrahydro-2H-pyran-4-yloxy)phenyl]pyrimidin-2-yl}amino)-N-[3-(dimethylamino)propyl]benzenesulfonamide

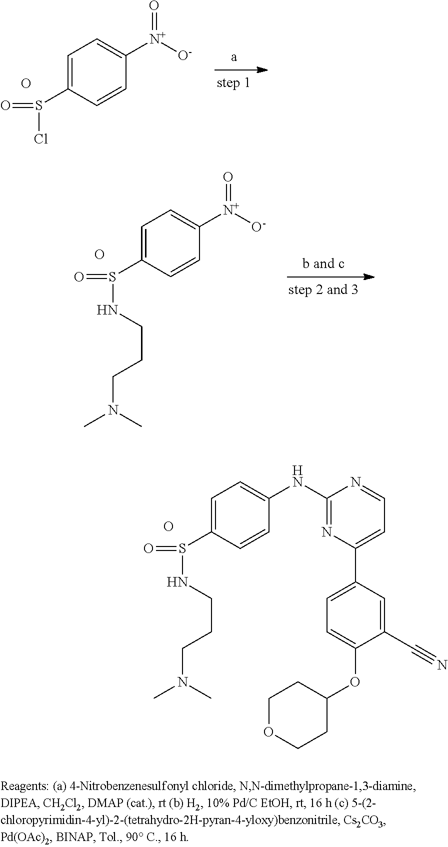

Step 1. N-[3-(Dimethylamino)propyl]-4-nitrobenzenesulfonamide: To a mixture of 4-nitrobenzenesulfonyl chloride (0.5 g, 2.25 mmol) and catalytic DMAP (0.01 g) in CH₂Cl₂ was added DIPEA (0.5 mL, 2.82 mmol) N,N-dimethylpropane-1,3-diamine (0.34 mL, 2.71 mmol). The reaction mixture was stirred at rt for 16 h, H₂O was added, and the layers separated and the aqueous layer extracted with CH₂Cl₂ (2×10 mL). The organic layers were combined, dried over sodium sulfate, filtered and evaporated under reduced pressure. The residue was purified by column chromatography (Hexanes/EtOAc) to afford the title compounds as an oil (0.40 g, 62%). ¹H NMR (DMSO-d₆) δ 8.43 (d, 2H), 8.04 (d, 2H), 2.82 (m, 2H), 2.28 (m, 2H), 2.14 (s, 6H), 1.53 (m, 2H); LC-MS [M+H]⁺ 188.

Step 2. 4-Amino-N-[3-(dimethylamino)propyl]benzenesulfonamide: To a N₂ (g) sparged solution of N-[3-(dimethylamino)propyl]-4-nitrobenzenesulfonamide (0.40 g, 1.16 mmol) in EtOH (20 mL) was added palladium on carbon (10%, 0.04 g). The reaction mixture was sparged with H₂ (g) and stirred at rt under atmospheric pressure of H₂ (g) for 16 h. The reaction mixture was filtered through Celite®, evaporated under reduced pressure to afford the crude intermediate which was used without further purification.

Step 3. 4-({4-[3-Cyano-4-(tetrahydro-2H-pyran-4-yloxy)phenyl]pyrimidin-2-yl}amino)-N-[3-(dimethylamino)propyl]benzenesulfonamide: The procedure used for the preparation of Intermediate I-11 was used to prepare the title compound from 4-amino-N-[3-(dimethylamino)propyl]benzenesulfonamide and 5-(2-chloropyrimidin-4-yl)-2-(tetrahydro-2H-pyran-4-yloxy)benzonitrile. ¹H NMR (DMSO-d₆) δ 10.2 (s, 1H), 9.30 (br s, 1H), 8.65-8.61 (m, 2H), 8.47 (dd, 1H), 8.41 (t, 1H), 8.00 (s, 1H), 7.88 (d, 1H), 7.60-7.56 (m, 2H), 7.38 (d, 1H), 4.96 (m, 1H), 4.00 (s, 3H), 3.90-3.85 (m, 2H), 3.67-3.62 (m, 2H), 3.60-3.54 (m, 2H), 3.29-3.24 (m, 2H), 2.85 (s, 6H), 2.08-2.01 (m, 2H), 1.73-1.66 (m, 2H); TOF [M+H]⁺ 537.2271.

### Example Compound 4

### 4-({4-[3-Cyano-4-({1-[(2R)-2-hydroxypropanoyl]piperidin-4-yl}oxy)phenyl]pyrimidin-2-yl}amino)-N-[3-(dimethylamino)propyl]benzamide

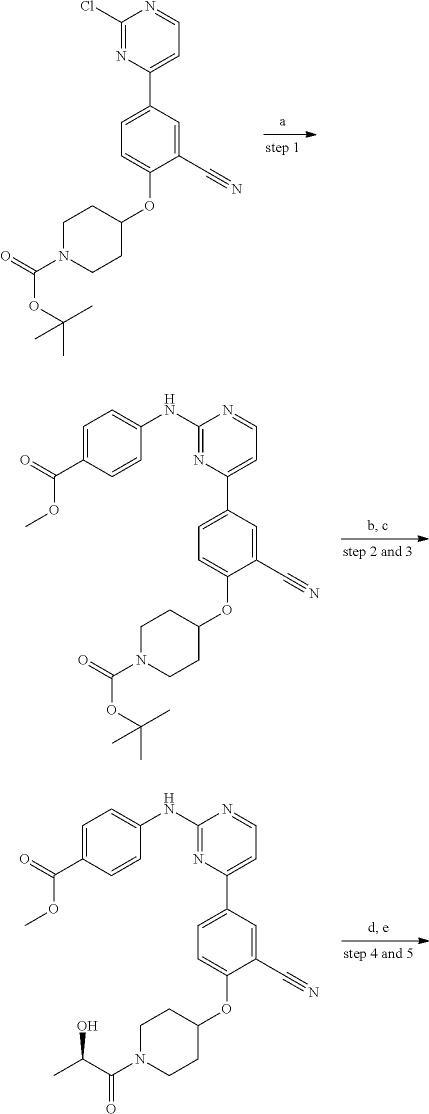 [SMILES: CC(C)(C)OC(=O)N1CCC(Oc2ccc(-c3ccnc(Cl)n3)cc2C#N)CC1 ; COC(=O)c1ccc(Nc2nccc(-c3ccc(OC4CCN(C(=O)OC(C)(C)C)CC4)c(C#N)c3)n2)cc1 ; COC(=O)c1ccc(Nc2nccc(-c3ccc(OC4CCN(C(=O)[C@@H](C)O)CC4)c(C#N)c3)n2)cc1]

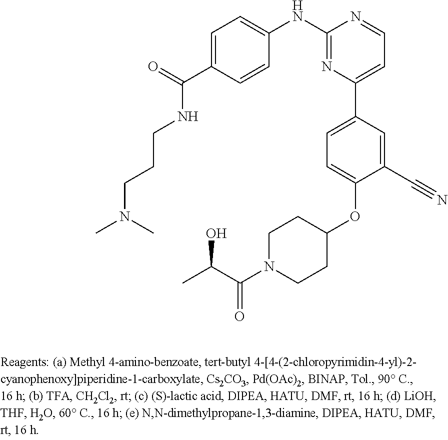 [SMILES: C[C@@H](O)C(=O)N1CCC(Oc2ccc(-c3ccnc(Nc4ccc(C(=O)NCCCN(C)C)cc4)n3)cc2C#N)CC1]

Step 1. tert-Butyl 4-[2-cyano-4-(2-{[4-(methoxycarbonyl)phenyl]amino}pyrimidin-4-yl)phenoxy]piperidine-1-carboxylate: Methyl 4-amino-benzoate (0.246 g, 1.63 mmol) and tert-butyl 4-[4-(2-chloropyrimidin-4-yl)-2-cyanophenoxy]piperidine-1-carboxylate (0.45 g, 1.08 mmol) were added to a flask. Cesium carbonate (1.77 g, 5.44 mmol) and p-dioxane (7.0 mL) were added and the reaction flask was flushed with nitrogen. Palladium acetate (0.036 g, 0.16 mmol) and BINAP (0.17 g, 0.27 mmol) were added and the reaction flask was flushed with nitrogen. The reaction mixture was placed in an oil bath at 90° C. and stirred for 16 h. The reaction was cooled to rt, H₂O (5 mL) and EtOAc (10 mL) were added and the layers were separated. The aqueous layer was extracted with EtOAc (2×10 mL), the organic layers were combined, dried over sodium sulfate, filtered, and concentrated in vacuo. Purification by column chromatography (EtOAc/Hexanes to EtOAc/20% MeOH in CH₂Cl₂ with 1% NH₄OH) afforded the title compound as a solid (0.4 g, 69%). ¹H NMR (DMSO-d₆) δ 10.2 (s, 1H), 8.64 (d, 1H), 8.58 (d, 1H), 8.50 (dd, 1H), 7.99-7.91 (m, 4H), 7.65-7.56 (m, 2H), 4.98-4.92 (m, 1H), 3.83 (s, 3H), 3.65-3.56 (m, 2H), 3.36-3.29 (m, 2H), 2.01-1.93 (m, 2H), 1.72-1.62 (m, 2H), 1.42 (s, 9H); LC-MS [M+H]⁺ 530.

Step 2. Methyl 4-({4-[3-cyano-4-(piperidin-4-yloxy)phenyl]pyrimidin-2-yl}amino)benzoate: A solution of tert-butyl 4-[2-cyano-4-(2-{[4-(methoxycarbonyl)phenyl]amino}pyrimidin-4-yl)phenoxy]piperidine-1-carboxylate (0.40 g, 0.76 mmol) in CH₂Cl₂ (20 mL) and trifluoroacetic acid (10 mL) was stirred at rt for 4 h. The solvent was evaporated under reduced pressure, aqueous sat. NaHCO₃ (20 mL) and CH₂Cl₂ (25 mL) were added and the layers separated. The aqueous layer was extracted with CH₂Cl₂ (5×25 mL), the organic layers combined, dried over sodium sulfate, filtered and the solvent evaporated under reduced pressure. Purification by column chromatography (EtOAc/Hexanes to EtOAc/20% MeOH in CH₂Cl₂ with 1% NH₄OH) afforded the title compound (0.30 g, 92%. ¹H NMR (DMSO-d₆) δ 10.2 (s, 1H), 8.65 (d, 1H), 8.60 (d, 1H), 8.51 (dd, 1H), 7.98-7.92 (m, 4H), 7.61-7.56 (m, 2H), 4.99-4.93 (m, 1H), 3.83 (s, 3H), 3.27-3.19 (m, 2H), 3.16-3.09 (m, 2H), 2.19-2.11 (m, 2H), 1.97-1.87 (m, 2H); LC-MS [M+H]⁺ 430.

Step 3. Methyl 4-({4-[3-cyano-4-({1-[(2R)-2-hydroxypropanoyl]piperidin-4-yl}oxy)phenyl]pyrimidin-2-yl}amino)benzoate: To a mixture of methyl 4-({4-[3-cyano-4-(piperidin-4-yloxy)phenyl]pyrimidin-2-yl}amino)benzoate (0.30 g, 0.70 mmol), (S)-lactic acid (0.105 g, 1.16 mmol) and DIPEA (0.205 mL, 1.16 mmol) in DMF (10 mL) was added HATU (0.44 g, 1.16 mmol). The reaction mixture was stirred for 16 h and purified by reverse phase chromatography (C₁₈, CH₃CN 95% in H₂O with 0.1% TFA). The desired fractions were collected and the solvent evaporated under reduced pressure to afford the title compound as the trifluoroacetate salt (0.35 g, 81%). ¹H NMR (DMSO-d₆) δ 10.2 (s, 1H), 8.64 (d, 1H), 8.58 (d, 1H), 8.53-8.49 (m, 1H), 8.00-7.92 (m, 4H), 7.61-7.58 (m, 2H), 5.06-4.95 (m, 1H), 4.51-4.44 (m, 1H), 3.83 (s, 3H), 3.78-3.68 (m, 2H), 3.58-3.46 (m, 2H), 2.08-1.92 (m, 2H), 1.80-1.65 (m, 2H), 1.25 (d, 3H); LC-MS [M+H]⁺ 502.

Step 4. 4-({4-[3-Cyano-4-({1-[(2R)-2-hydroxypropanoyl]piperidin-4-yl}oxy)phenyl]pyrimidin-2-yl}amino)benzoic acid: To a solution of methyl 4-({4-[3-cyano-4-({1-[(2R)-2-hydroxypropanoyl]piperidin-4-yl}oxy)phenyl]pyrimidin-2-yl}amino)benzoate trifluoroacetate salt (0.35 g, 0.57 mmol) in THF/H₂O (2:1, 30 mL) was added LiOH (0.83 g, 3.49 mmol). The reaction mixture was stirred at 60° C. for 16 h. The solvent was evaporated and the residue purified by reverse phase chromatography (C₁₈, CH₃CN 95% in H₂O with 0.1% TFA) to afford the title compound as the trifluoroacetate salt (0.4 g, quant.).

Step 5. 4-({4-[3-Cyano-4-({1-[(2R)-2-hydroxypropanoyl]piperidin-4-yl}oxy)phenyl]pyrimidin-2-yl}amino)-N-[3-(dimethylamino)propyl]benzamide: To a mixture of 4-({4-[3-cyano-4-({1-[(2R)-2-hydroxypropanoyl]piperidin-4-yl}oxy)phenyl]pyrimidin-2-yl}amino)benzoic acid (0.10 g, 0.205 mmol), N,N-dimethylpropane-1,3-diamine (0.02 mL), 0.256 mmol) and DIPEA (0.050 mL, 0.267 mmol) in DMF (2 mL) was added HATU (0.100 g, 0.256 mmol). The reaction mixture was stirred for 16 h. The solvent was evaporated and the residue purified by reverse phase chromatography (C₁₈, CH₃CN 95% in H₂O with 0.1% TFA). The desired fractions were collected and the solvent evaporated under reduced pressure. The resulting solid was recrystallized from EtOAc/Hexanes to afford the title compound as the trifluoroacetate salt (0.014 g, 10%). ¹H NMR (DMSO-d₆) δ 10.2 (s, 1H), 9.44 (br s, 1H), 8.62 (d, 1H), 8.57 (d, 1H), 8.53-8.49 (m, 1H), 7.93-7.83 (m, 4H), 7.59-7.56 (m, 2H), 5.06-4.98 (m, 2H), 4.50-4.44 (m, 1H), 3.86-3.66 (m, 2H), 3.58-3.48 (m, 2H), 3.36-3.30 (m, 2H), 3.14-3.04 (m, 1H), 2.80 (s, 3H), 2.79 (s, 3H), 2.14-1.95 (m, 2H), 1.92-1.85 (m, 2H), 1.80-1.58 (m, 2H), 1.21 (d, 3H); TOF [M+H]⁺ 572.2979.

### Example Compound 5

### 5-[2-[(4-Morpholinophenyl)amino]pyrimidin-4-yl]-2-tetrahydropyran-4-yloxy-benzonitrile

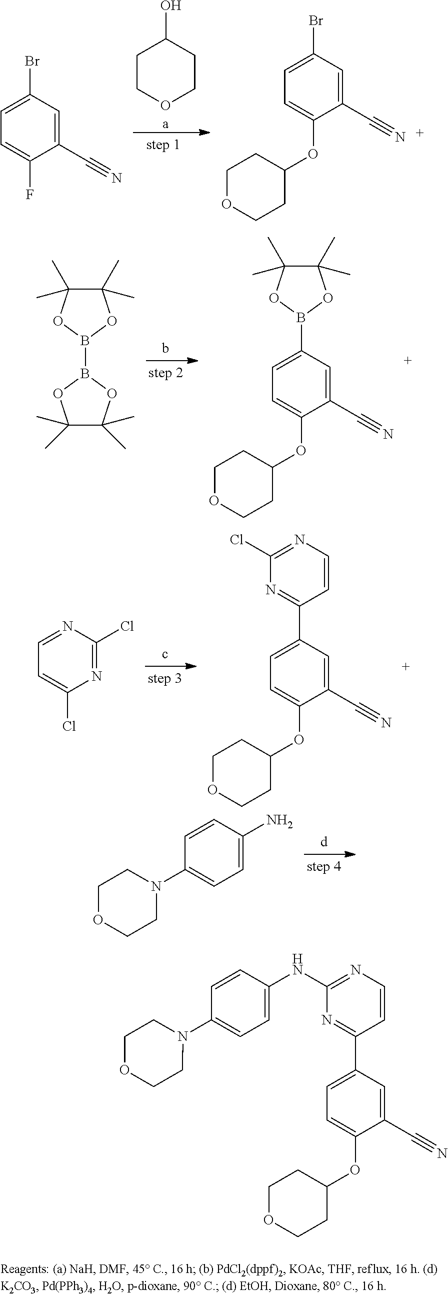

Step 1: 5-Bromo-2-tetrahydropyran-4-yloxy-benzonitrile: To tetrahyropyranol (7.1 g, 69.5 mmol) in DMF (130 mL) at 0° C. was added NaH (2.78 g, 69.5 mmol). 5-bromo-2-fluorobenzonitrile (11.6 g, 57.9 mmol) was added dropwise as a solution in DMF (63 mL). The reaction was stirred at 45° C. for 16 h. The reaction was cooled to rt and quenched by pouring the reaction into H₂O (1.5 L). The precipitate was filtered and dried under vacuum to provide 16.8 g of material (88%). The product was used without further purification. ¹H NMR (DMSO) δ 8.02 (s, 1H), 7.82 (d, 1H), 7.35 (d, 1H), 4.85-4.76 (m, 1H), 3.90-3.80 (m, 2H), 3.58-3.49 (m, 2H), 2.04-1.95 (m, 2H), 1.70-1.60 (m, 2H).

Step 2: 2-Tetrahydropyran-4-yloxy-5-(4,4,5,5-tetramethyl-1,3,2-dioxaborolan-2-yl)benzonitrile: To 5-Bromo-2-tetrahydropyran-4-yloxy-benzonitrile (7.8 g, 23.5 mmol) in p-dioxane (78 mL) was added bis(pinacolato)diboron (8.9 g, 35.3 mmol), KOAc (6.9 g, 70.5 mmol), and Pd(dppf)Cl₂ (0.86 g, 1.2 mmol). The reaction was heated to 90° C. for 16 h. The reaction was quenched with H₂O (50 mL), followed by extraction with EtOAc (3×25 mL). The aqueous and organic layers were separated. The organic layer was washed with aq. saturated NaCl and dried (Na₂SO₄). Purification by medium pressure liquid chromatography (0-100% EtOAc in Hexanes) provided 7.6 g (98%) material. ¹H NMR (CDCl₃) δ 8.04 (s, 1H), 7.90 (d, 1H), 6.95 (d, 1H), 4.77-4.70 (m, 1H), 4.10-4.00 (m, 2H), 3.67-3.60 (m, 2H), 2.10-2.00 (m, 2H), 1.90-1.81 (m, 2H), 1.15 (s, 12H).

Step 3: 5-(2-chloropyrimidin-4-yl)-2-tetrahydropyran-4-yloxy-benzonitrile: To 2-tetrahydropyran-4-yloxy-5-(4,4,5,5-tetramethyl-1,3,2-dioxaborolan-2-yl)benzonitrile (8.0 g, 24.3 mmol) in p-dioxane (60 mL) and H₂O (20 mL) was added 2,4-dichloropyrimidine (3.6 g, 24.3 mmol), K₂CO₃ (6.7 g, 48.6 mmol), and Pd(PPh₃)₄ (1.4 g, 1.2 mmol). The reaction was heated to 90° C. for 16 h. The reaction was quenched with H₂O (50 mL) followed by extraction with EtOAc (3×25 mL). The aqueous and organic layers were separated. The organic layer was washed with aq. saturated NaCl and dried (Na₂SO₄). Purification by medium pressure liquid chromatography (0-100% EtOAc in Hexanes) provided 7.5 g (98%) material. ¹H NMR (CDCl₃) δ 8.66 (d, 1H), 8.35-8.29 (m, 2H), 7.65 (d, 1H), 7.05 (d, 1H), 4.82-4.85 (m, 1H), 4.10-4.00 (m, 2H), 3.71-3.62 (m, 2H), 2.15-2.05 (m, 2H), 1.99-1.89 (m, 2H).

Step 4: 5-[2-[(4-morpholinophenyl)amino]pyrimidin-4-yl]-2-tetrahydropyran-4-yloxy-benzonitrile: To 5-(2-chloropyrimidin-4-yl)-2-tetrahydropyran-4-yloxy-benzonitrile (9 g, 28.5 mmol) in EtOH (42 mL) and p-dioxane (42 mL) was added 4-morpholinoaniline (5.6 g, 31.3 mmol). The reaction was heated to 80° C. and stirred under N₂ (g) for three days. The solvent was removed under vacuum. The product was dissolved in warm (55° C.) MeOH (25 mL). The solution was cooled to room temperature. The product precipitated to provide 13 g (100%) material. ¹H NMR (DMSO) δ 9.90 (br s, 1H), 8.58-8.54 (m, 2H), 8.45 (d, 2H), 7.84-7.80 (m, 2H), 7.58-7.50 (m, 3H), 5.00-4.90 (m, 1H), 4.05-3.95 (m, 4H), 3.91-3.84 (m, 2H), 3.60-3.52 (m, 2H), 3.48-3.36 (m, 4H), 2.10-2.00 (m, 2H), 1.75-1.65 (m, 2H). LCMS [M+H]⁺ 458.2251.

### Example Compound 6

### 2-({1-[(2S)-2-Hydroxypropanoyl]piperidin-4-yl}oxy)-5-(2-{[4-(morpholin-4-yl)phenyl]amino}pyrimidin-4-yl)benzonitrile

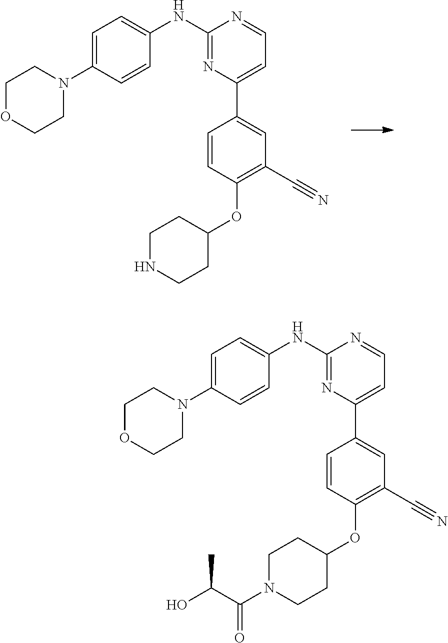 [SMILES: N#Cc1cc(-c2ccnc(Nc3ccc(N4CCOCC4)cc3)n2)ccc1OC1CCNCC1 ; C[C@H](O)C(=O)N1CCC(Oc2ccc(-c3ccnc(Nc4ccc(N5CCOCC5)cc4)n3)cc2C#N)CC1]

To a solution of tert-Butyl 4-[2-cyano-4-(2-{[4-(morpholin-4-yl)phenyl]amino}pyrimidin-4-yl)phenoxy]piperidine-1-carboxylate (0.100 g, 0.22 mmol) in CH₂Cl₂ (5 mL) was added Et₃N (0.1 mL, 0.756 mmol) and HBTU (0.100 g, 0.264 mmol) and L-lactic acid (0.024 g, 0.264 mmol) at rt. After stirring for 18 h, the mixture was concentrated, and the residue was purified by column chromatography (SiO₂, MeOH 020% in CH₂Cl₂ with 0.1% NH₄OH) to give the title compound.

### Example Compound 7

### 1-(4-{[4-(3-Cyano-4-methoxyphenyl)pyrimidin-2-yl]amino}phenyl)-3-(3-hydroxypropyl)urea

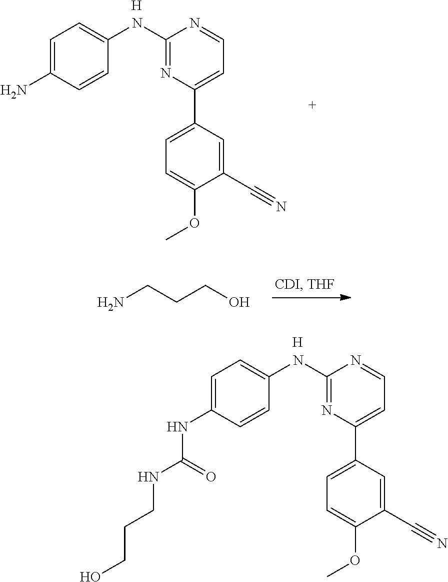

A solution of 5-{2-[(4-aminophenyl)amino]pyrimidin-4-yl}-2-methoxybenzonitrile (80 mg, 0.25 mmol) and carbonyldimidazole (48 mg, 0.30 mmol) in THF (2 mL) was stirred for 1 h. 3-Aminopropan-1-ol (100 μL) was added and the reaction stirred for 2 h. The reaction was concentrated onto Celite® and purified by RP-MPLC (C₁₈, MeOH/H₂O, 0-100%, w/0.1% TFA) to provide the title compound. ¹H NMR (DMSO-d₆) δ 9.55 (s, 1H), 8.53 (d, 1H), 8.52-8.46 (m, 2H), 8.36 (br s, 1H), 7.65-7.57 (m, 2H), 7.45 (d, 1H), 7.42 (d, 1H), 7.37-7.30 (m, 2H), 6.08 (br s, 1H), 4.01 (s, 3H), 3.46 (t, 2H), 3.14 (t, 2H), 1.58 (quint, 2H); LC-MS [M+H]⁺ 419.1829.

### Example Compound 8

### 1-(4-{[4-(3-Cyano-4-methoxyphenyl)pyrimidin-2-yl]amino}phenyl)-3-cyclopentylurea

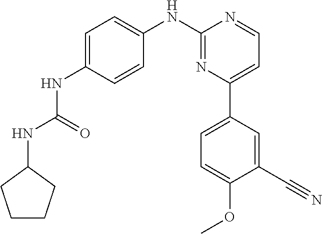 [SMILES: COc1ccc(-c2ccnc(Nc3ccc(NC(=O)NC4CCCC4)cc3)n2)cc1C#N]

The procedure used in the preparation of Example Compound 7 was used to prepare the title compound from 5-[2-[(4-aminophenyl)amino]pyrimidin-4-yl]-2-methoxy-benzonitrile and cyclopentanamine. ¹H NMR (DMSO-d₆) δ 9.51 (s, 1H), 8.52 (d, 1H), 8.51-8.46 (m, 2H), 8.14 (s, 1H), 7.65-7.58 (m, 2H), 7.45 (d, 1H), 7.41 (d, 1H), 7.35-7.28 (m, 2H), 6.08 (d, 1H), 4.01 (s, 3H), 3.93 (sextet, 1H), 1.90-1.75 (m, 2H), 1.70-1.45 (m, 4H), 1.40-1.28 (m, 2H); LC-MS [M+H]⁺ 429.2035.

### Example Compound 9

### 1-(4-{[4-(3-Cyano-4-methoxyphenyl)pyrimidin-2-yl]amino}phenyl)-3-(2-hydroxyethyl)urea

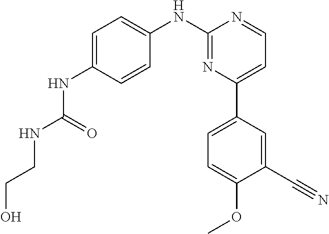 [SMILES: COc1ccc(-c2ccnc(Nc3ccc(NC(=O)NCCO)cc3)n2)cc1C#N]

The procedure used in the preparation of Example Compound 7 was used to prepare the title compound from 5-[2-[(4-aminophenyl)amino]pyrimidin-4-yl]-2-methoxy-benzonitrile and 2-aminoethanol. ¹H NMR (DMSO-d₆) δ 9.55 (s, 1H), 8.53 (d, 1H), 8.52-8.42 (m, 3H), 7.68-7.58 (m, 2H), 7.45 (d, 1H), 7.42 (d, 1H), 7.36-7.31 (m, 2H), 6.13 (br s, 1H), 4.01 (s, 3H), 3.44 (t, 2H), 3.15 (t, 2H); LC-MS [M+H]⁺ 405.1669.

### Example Compound 10

### 1-(3-Aminopropyl)-3-(4-{[4-(3-cyano-4-methoxyphenyl)pyrimidin-2-yl]amino}phenyl)urea

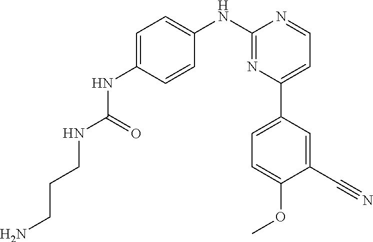 [SMILES: COc1ccc(-c2ccnc(Nc3ccc(NC(=O)NCCCN)cc3)n2)cc1C#N]

The procedure used in the preparation of Example Compound 7 was used to prepare the title compound from 5-[2-[(4-aminophenyl)amino]pyrimidin-4-yl]-2-methoxy-benzonitrile and propane-1,3-diamine. ¹H NMR (DMSO-d₆) δ 9.55 (s, 1H), 8.55-8.45 (m, 4H), 7.70 (br s, 3H), 7.62 (d, 2H), 7.48-7.40 (m, 2H), 7.34 (d, 2H), 6.32 (br s, 1H), 4.01 (s, 3H), 3.20-3.10 (m, 2H), 2.88-2.76 (m, 2H), 1.71 (quint, 2H); LC-MS [M+H]⁺ 418.1990.

### Example Compound 11

### 1-[4-({4-[3-Cyano-4-(tetrahydro-2H-pyran-4-yloxy)phenyl]pyrimidin-2-yl}amino)phenyl]-3-(2-hydroxyethyl)urea

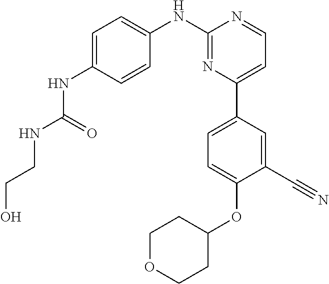 [SMILES: N#Cc1cc(-c2ccnc(Nc3ccc(NC(=O)NCCO)cc3)n2)ccc1OC1CCOCC1]

The procedure used in the preparation of Example Compound 7 was used to prepare the title compound from 5-[2-[(4-aminophenyl)amino]pyrimidin-4-yl]-2-tetrahydropyran-4-yloxy-benzonitrile and 2-aminoethanol. ¹H NMR (DMSO-d₆) δ 9.54 (s, 1H), 8.56-8.54 (m, 1H), 8.54-8.46 (m, 1H), 8.45-8.42 (m, 2H), 7.65-7.60 (m, 2H), 7.55 (d, 1H), 7.42 (s, 1H), 7.36-7.30 (m, 2H), 6.14 (br s, 1H), 4.94 (sept, 1H), 3.94-3.84 (m, 2H), 3.55 (ddd, 2H), 3.44 (t, 2H), 3.19-3.10 (m, 2H), 2.10-2.00 (m, 2H), 1.75-1.62 (m, 2H); LC-MS [M+H]⁺ 475.2079.

### Example Compound 12

### 5-[2-(Phenylamino)pyrimidin-4-yl]-2-(tetrahydro-2H-pyran-4-yloxy)benzonitrile

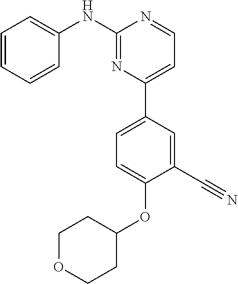 [SMILES: N#Cc1cc(-c2ccnc(Nc3ccccc3)n2)ccc1OC1CCOCC1]

The procedure used in the preparation of Intermediate I-11 was used to prepare the title compound from 5-(2-chloropyrimidin-4-yl)-2-tetrahydropyran-4-yloxy-benzonitrile and aniline. ¹H NMR (DMSO-d₆) δ 9.71 (s, 1H), 8.58-8.53 (m, 2H), 8.46 (dd, 1H), 7.83-7.78 (m, 2H), 7.56 (s, 1H), 7.48 (d, 1H), 7.35-7.28 (m, 2H), 7.01-6.95 (m, 1H), 4.95 (sept, 1H), 3.94-3.82 (m, 2H), 3.56 (ddd, 2H), 2.10-2.00 (m, 2H), 1.75-1.63 (m, 2H); LC-MS [M+H]⁺ 373.1592.

### Example Compound 13

### N-[4-({4-[3-Cyano-4-(tetrahydro-2H-pyran-4-yloxy)phenyl]pyrimidin-2-yl}amino)phenyl]morpholine-4-carboxamide

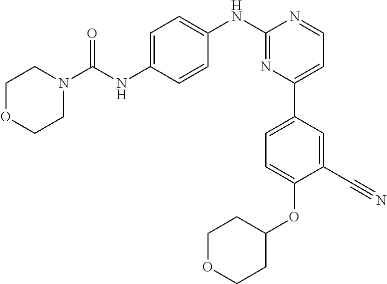 [SMILES: N#Cc1cc(-c2ccnc(Nc3ccc(NC(=O)N4CCOCC4)cc3)n2)ccc1OC1CCOCC1]

The procedure used in the preparation of Example Compound 7 was used to prepare the title compound from 5-[2-[(4-aminophenyl)amino]pyrimidin-4-yl]-2-tetrahydropyran-4-yloxy-benzonitrile and morpholine. ¹H NMR (DMSO-d₆) δ 9.58 (s, 1H), 8.53 (d, 1H), 8.51 (d, 1H), 8.48-8.42 (m, 2H), 7.68-7.62 (m, 2H), 7.56 (d, 1H), 7.43 (d, 1H), 7.42-4.36 (m, 2H), 4.94 (sept, 1H), 3.92-3.83 (m, 2H), 3.58-3.65 (m, 4H), 3.55 (ddd, 2H), 3.45-3.38 (m, 4H), 2.10-1.98 (m, 2H), 1.62-1.76 (m, 2H); LC-MS [M+H]⁺ 501.2185.

### Example Compound 14

### 1-[4-({4-[3-Cyano-4-(tetrahydro-2H-pyran-4-yloxy)phenyl]pyrimidin-2-yl}amino)phenyl]-3-pyridin-3-ylurea

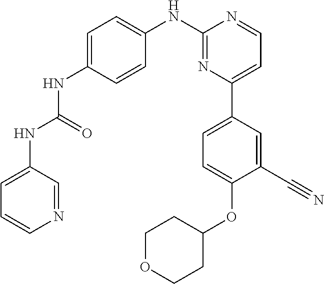 [SMILES: N#Cc1cc(-c2ccnc(Nc3ccc(NC(=O)Nc4cccnc4)cc3)n2)ccc1OC1CCOCC1]

The procedure used in the preparation of Example Compound 7 was used to prepare the title compound from 5-[2-[(4-aminophenyl)amino]pyrimidin-4-yl]-2-tetrahydropyran-4-yloxy-benzonitrile and pyridin-3-amine. ¹H NMR (DMSO-d₆) δ 9.68 (s, 1H), 9.65 (s, 1H), 9.22 (s, 1H), 9.04 (s, 1H), 8.57-8.52 (m, 2H), 8.48-8.42 (m, 2H), 8.30-8.25 (m, 1H), 7.82 (dd, 1H), 7.76-7.71 (m, 2H), 7.57 (d, 1H), 7.47-7.41 (m, 3H), 4.95 (sept, 1H), 3.92-3.84 (m, 2H), 3.56 (ddd, 2H), 2.10-2.00 (m, 2H), 1.76-1.63 (m, 2H); LC-MS [M+H]⁺ 508.2116.

### Example Compound 15

### 5-[2-(1,3-Benzothiazol-5-ylamino)pyrimidin-4-yl]-2-(tetrahydro-2H-pyran-4-yloxy)benzonitrile

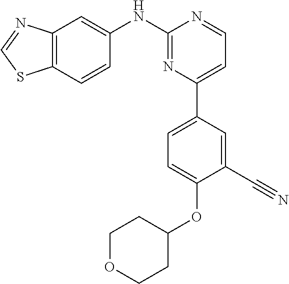 [SMILES: N#Cc1cc(-c2ccnc(Nc3ccc4scnc4c3)n2)ccc1OC1CCOCC1]

The procedure used in the preparation of Intermediate I-11 was used to prepare the title compound from 5-(2-chloropyrimidin-4-yl)-2-tetrahydropyran-4-yloxy-benzonitrile and 1,3-benzothiazol-5-amine. The title compound was purified by MPLC (SiO₂, EtOAc/Hexanes, 0-100%) followed by RP-MPLC (C₁₈, MeOH/H₂O, 0-100%, w/0.1% TFA). ¹H NMR (DMSO-d₆) δ 9.99 (s, 1H), 9.37 (s, 1H), 8.76 (d, 1H), 8.62 (d, 1H), 8.58 (d, 1H), 8.49 (dd, 1H), 8.06 (d, 1H), 7.81 (dd, 1H), 7.59 (d, 1H), 7.54 (d, 1H), 4.97 (sept, 1H), 3.92-3.83 (m, 2H), 3.56 (ddd, 2H), 2.10-2.00 (m, 2H), 1.76-1.62 (m, 2H); LC-MS [M+H]⁺ 430.1328.

### Example Compound 16

### 5-[2-(1,3-Benzothiazol-6-ylamino)pyrimidin-4-yl]-2-(tetrahydro-2H-pyran-4-yloxy)benzonitrile

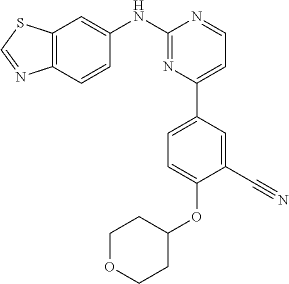 [SMILES: N#Cc1cc(-c2ccnc(Nc3ccc4ncsc4c3)n2)ccc1OC1CCOCC1]

The procedure used in the preparation of Intermediate I-11 was used to prepare the title compound from 5-(2-chloropyrimidin-4-yl)-2-tetrahydropyran-4-yloxy-benzonitrile and 1,3-benzothiazol-6-amine. The title compound was purified by MPLC (SiO₂, EtOAc/Hexanes, 0-100%) followed by RP-MPLC (C₁₈, MeOH/H₂O, 0-100%, w/0.1% TFA). ¹H NMR (DMSO-d₆) δ 10.04 (s, 1H), 9.23 (s, 1H), 8.76 (d, 1H) 8.62 (d, 1H), 8.58 (d, 1H), 8.47 (dd, 1H), 8.02 (d, 1H), 7.81 (dd, 1H), 7.57 (dd, 1H), 7.54 (d, 1H), 4.96 (sept, 1H), 3.94-3.83 (m, 2H), 3.56 (ddd, 2H), 2.10-1.98 (m, 2H), 1.76-1.63 (m, 2H); LC-MS [M+H]⁺ 430.1334.

### Example Compound 17

### 1-[4-({4-[3-Cyano-4-(tetrahydro-2H-pyran-4-yloxy)phenyl]pyrimidin-2-yl}amino)phenyl]-3-pyridin-4-ylurea

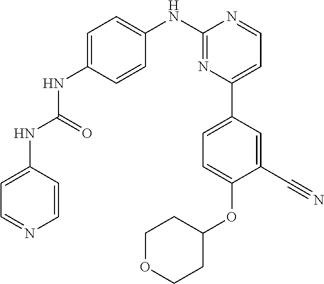 [SMILES: N#Cc1cc(-c2ccnc(Nc3ccc(NC(=O)Nc4ccncc4)cc3)n2)ccc1OC1CCOCC1]

The procedure used in the preparation of Example Compound 7 was used to prepare the title compound from 5-[2-[(4-aminophenyl)amino]pyrimidin-4-yl]-2-tetrahydropyran-4-yloxy-benzonitrile and pyridin-4-amine. ¹H NMR (DMSO-d₆) δ 11.04 (s, 1H), 9.92 (s, 1H), 9.70 (s, 1H), 8.61 (d, 2H), 8.57-8.52 (m, 2H), 8.46 (dd, 1H), 8.02-7.92 (m, 2H), 7.82-7.73 (m, 2H), 7.57 (d, 1H), 7.54-7.43 (m, 3H), 4.95 (sept, 1H), 3.94-3.82 (m, 2H), 3.62-3.50 (m, 2H), 1.97-2.04 (m, 2H), 1.78-1.60 (m, 2H); LC-MS [M+H]⁺ 508.2114.

### Example Compound 18

### 5-(2-{[3-Methyl-4-(morpholin-4-yl)phenyl]amino}pyrimidin-4-yl)-2-(tetrahydro-2H-pyran-4-yloxy)benzonitrile

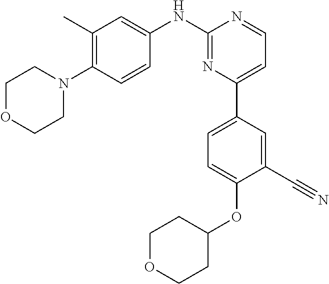 [SMILES: Cc1cc(Nc2nccc(-c3ccc(OC4CCOCC4)c(C#N)c3)n2)ccc1N1CCOCC1]

The procedure used in the preparation of Intermediate I-11 was used to prepare the title compound from 5-(2-chloropyrimidin-4-yl)-2-tetrahydropyran-4-yloxy-benzonitrile and 3-methyl-4-morpholino-aniline. ¹H NMR (DMSO-d₆) δ 9.57 (s, 1H), 8.55 (d, 1H), 5.20 (d, 1H), 8.44 (dd, 1H), 7.52-7.68 (m, 3H), 7.44 (d, 1H), 7.04 (d, 1H), 4.95 (sept, 1H), 3.92-3.83 (m, 2H), 3.79-3.71 (m, 4H), 3.56 (ddd, 2H), 2.84 (br s, 4H), 2.30 (s, 3H), 2.10-2.00 (m, 2H), 1.75-1.62 (m, 2H); LC-MS [M+H]⁺ 472.2332.

### Example Compound 19

### 4-Acetyl-N-[4-({4-[3-cyano-4-(tetrahydro-2H-pyran-4-yloxy)phenyl]pyrimidin-2-yl}amino)phenyl]piperazine-1-carboxamide

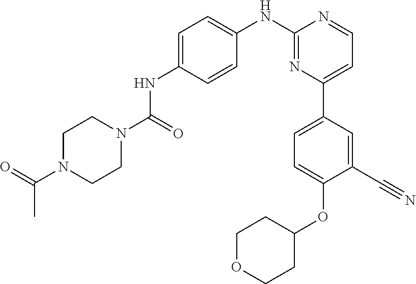 [SMILES: CC(=O)N1CCN(C(=O)Nc2ccc(Nc3nccc(-c4ccc(OC5CCOCC5)c(C#N)c4)n3)cc2)CC1]

The procedure used in the preparation of Example Compound 7 was used to prepare the title compound from 5-[2-[(4-aminophenyl)amino]pyrimidin-4-yl]-2-tetrahydropyran-4-yloxy-benzonitrile and 1-piperazin-1-ylethanone. ¹H NMR (DMSO-d₆) δ 9.59 (s, 1H), 8.56-8.50 (m, 3H), 8.44 (dd, 1H), 7.68-7.62 (m, 2H), 7.56 (d, 1H), 7.43 (d, 1H), 7.42-7.36 (m, 2H), 4.94 (sept, 1H), 3.92-3.83 (m, 2H), 3.55 (ddd, 2H), 3.47 (br s, 6H), 3.46-3.38 (m, 2H), 2.10-2.00 (m, 2H), 2.04 (s, 3H), 1.76-1.62 (m, 2H); LC-MS [M+H]⁺ 542.2510.

### Example Compound 20

### N-[4-({4-[3-Cyano-4-(tetrahydro-2H-pyran-4-yloxy)phenyl]pyrimidin-2-yl}amino)phenyl]-4-methylpiperazine-1-carboxamide

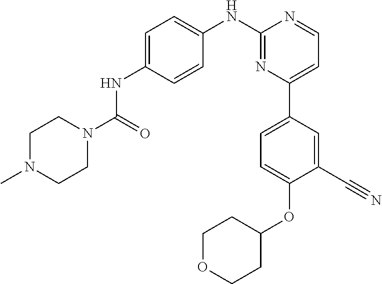 [SMILES: CN1CCN(C(=O)Nc2ccc(Nc3nccc(-c4ccc(OC5CCOCC5)c(C#N)c4)n3)cc2)CC1]

The procedure used in the preparation of Example Compound 7 was used to prepare the title compound from 5-[2-[(4-aminophenyl)amino]pyrimidin-4-yl]-2-tetrahydropyran-4-yloxy-benzonitrile and 1-methylpiperazine. ¹H NMR (DMSO-d₆) δ 9.84 (br s, 1H), 9.61 (s, 1H), 8.69 (s, 1H), 8.55-8.50 (m, 2H), 8.44 (dd, 1H), 7.67 (d, 2H), 7.55 (d, 1H), 7.44 (d, 1H), 7.38 (d, 1H), 4.95 (sept, 1H), 4.25 (d, 2H), 3.94-3.82 (m, 2H), 3.56 (ddd, 2H), 3.47 (d, 2H), 3.20-2.95 (m, 5H), 2.84 (s, 3H), 2.10-1.98 (m, 2H), 1.78-1.62 (m, 2H); LC-MS [M+H]⁺ 514.2549.

### Example Compound 21

### 5-[2-({4-[2-(2-Aminoethoxy)ethoxy]-3-methoxyphenyl}amino)pyrimidin-4-yl]-2-(tetrahydro-2H-pyran-4-yloxy)benzonitrile

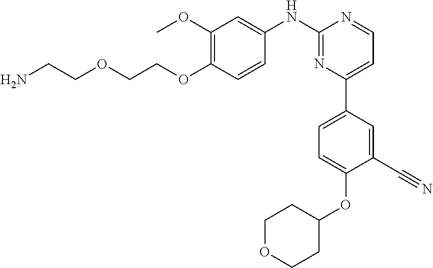 [SMILES: COc1cc(Nc2nccc(-c3ccc(OC4CCOCC4)c(C#N)c3)n2)ccc1OCCOCCN]

Standard Method E, BOC Deprotection was used to prepare the title compound from tert-butyl N-[2-[2-[4-[[4-(3-cyano-4-tetrahydropyran-4-yloxy-phenyl)pyrimidin-2-yl]amino]-2-methoxy-phenoxy]ethoxy]ethyl]carbamate. ¹H NMR (DMSO-d₆) δ 9.58 (br s, 1H), 8.56 (d, 1H), 8.53 (d, 1H), 8.43 (dd, 1H), 7.81 (br s, 3H), 7.70 (br s, 1H, 7.54 (d, 1H), 7.44 (d, 1H), 7.20 (d, 1H), 6.94 (d, 1H), 4.95 (sept, 1H), 4.12-4.06 (m, 2H), 3.92-3.84 (m, 2H), 3.82 (s, 3H), 3.82-3.76 (m, 2H), 3.71-3.66 (m, 2H), 3.56 (ddd, 2H), 3.08-2.98 (m, 2H), 2.1-2.0 (m, 2H), 1.75-1.61 (m, 2H); LC-MS [M+H]⁺ 506.2394.

### Example Compound 22

### N-(2-{2-[4-({4-[3-Cyano-4-(tetrahydro-2H-pyran-4-yloxy)phenyl]pyrimidin-2-yl}amino)-2-methoxyphenoxy]ethoxy}ethyl)methanesulfonamide

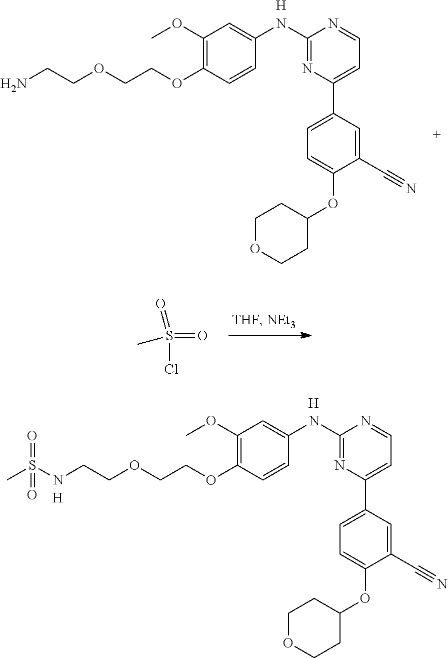

A solution of 5-[2-({4-[2-(2-aminoethoxy)ethoxy]-3-methoxyphenyl}amino) pyrimidin-4-yl]-2-(tetrahydro-2H-pyran-4-yloxy)benzonitrile (22.2 mg, 0.044 mmol), Et₃N (0.25 mL) in THF (2 mL) was treated with methanesulfonyl chloride (4 μL, 0.051 mmol) for 2 h. The reaction was concentrated onto Celite® and purified by RP-MPLC (C₁₈, MeOH/H₂O, 0-100% w/0.1% TFA) to provide the title compound. ¹H NMR (DMSO-d₆) δ 9.56 (s, 1H), 8.56 (d, 1H), 8.52 (d, 1H), 8.44 (dd, 1H), 7.65 (br s, 1H), 7.55 (d, 1H), 7.43 (d, 1H), 7.20 (d, 1H), 7.09 (t, 1H), 6.92 (d, 1H), 4.95 (sept, 1H), 4.07-4.03 (m, 2H), 3.91-3.84 (m, 2H), 3.81 (s, 3H), 3.76-3.72 (m, 2H), 3.60-3.52 (m, 4H), 3.14 (q, 2H), 2.93 (s, 3H), 2.10-1.98 (m, 2H), 1.75-1.61 (m, 2H); LC-MS [M+H]⁺ 584.2170.

### Example Compound 23

### 5-[2-(1,3-Benzodioxol-5-ylamino)pyrimidin-4-yl]-2-(tetrahydro-2H-pyran-4-yloxy)benzonitrile

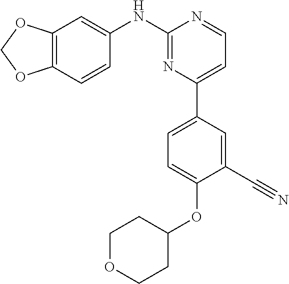 [SMILES: N#Cc1cc(-c2ccnc(Nc3ccc4c(c3)OCO4)n2)ccc1OC1CCOCC1]

The procedure used in the preparation of Intermediate I-11 was used to prepare the title compound from 5-(2-chloropyrimidin-4-yl)-2-tetrahydropyran-4-yloxy-benzonitrile and 1,3-benzodioxol-5-amine. ¹H NMR (DMSO-d₆) δ 9.60 (s, 1H), 8.54-8.52 (m, 2H), 8.42 (dd, 1H), 7.56 (d, 1H), 7.53 (d, 1H), 7.44 (d, 1H), 7.16 (dd, 1H), 6.87 (d, 1H), 5.99 (s, 2H), 4.95 (sept, 1H), 3.93-3.83 (m, 2H), 3.55 (ddd, 2H), 2.10-1.98 (m, 2H), 1.74-1.62 (m, 2H); LC-MS [M+H]⁺ 417.1546.

### Example Compound 24

### 5-(2-{[3-Fluoro-4-(morpholin-4-yl)phenyl]amino}pyrimidin-4-yl)-2-(tetrahydro-2H-pyran-4-yloxy)benzonitrile

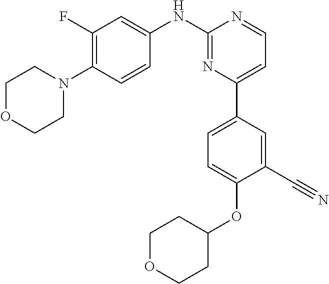 [SMILES: N#Cc1cc(-c2ccnc(Nc3ccc(N4CCOCC4)c(F)c3)n2)ccc1OC1CCOCC1]

The procedure used for the preparation of Intermediate I-11 was used to prepare the title compound from 5-(2-chloropyrimidin-4-yl)-2-tetrahydropyran-4-yloxy-benzonitrile and 3-fluoro-4-morpholino-aniline. ¹H NMR (DMSO-d₆) 9.77 (s, 1H), 8.55 (d, 1H), 8.54 (d, 1H), 8.44 (dd, 1H), 7.78 (dd, 1H), 7.56 (d, 1H), 7.56-7.44 (m, 2H), 7.02 (dd, 1H), 4.95 (sept, 1H), 3.92-3.82 (m, 2H), 3.78-3.70 (m, 4H), 3.56 (ddd, 2H), 3.00-2.92 (m, 4H), 2.10-2.00 (m, 2H), 1.75-1.63 (m, 2H); LC-MS [M+H]⁺ 476.2079.

### Example Compound 25

### 5-{2-[(3-Methoxy-4-{3-[(4-methylpiperazin-1-yl)sulfonyl]propoxy}phenyl)amino]pyrimidin-4-yl}-2-(tetrahydro-2H-pyran-4-yloxy)benzonitrile

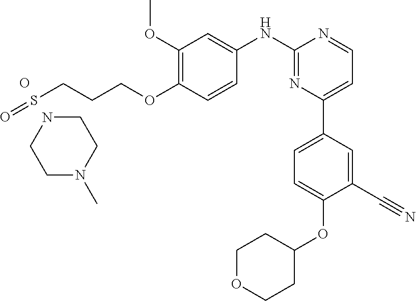

The procedure used for the preparation of Intermediate I-11 was used to prepare the title compound from 5-(2-chloropyrimidin-4-yl)-2-tetrahydropyran-4-yloxy-benzonitrile and 3-methoxy-4-[3-(4-methylpiperazin-1-yl)sulfonylpropoxy]aniline. ¹H NMR (DMSO-d₆) δ 9.96 (br s, 1H), 9.60 (s, 1H), 8.56 (d, 1H), 8.53 (d, 1H), 8.44 (dd, 1H), 7.70 (br s, 1H), 7.55 (d, 1H), 7.44 (d, 1H), 7.21 (d, 1H), 6.94 (d, 1H), 4.95 (sept, 1H), 4.04 (t, 2H), 3.92-3.75 (m, 4H), 3.83 (s, 3H), 3.60-3.47 (m, 4H), 3.39-3.31 (m, 2H), 3.22-3.04 (m, 4H), 2.85 (s, 3H), 2.15-1.98 (m, 4H), 1.75-1.62 (m, 2H); LC-MS [M+H]⁺ 623.2646.

### Example Compound 26

### N′-(2-{2-[4-({4-[3-Cyano-4-(tetrahydro-2H-pyran-4-yloxy)phenyl]pyrimidin-2-yl}amino)-2-methoxyphenoxy]ethoxy}ethyl)-N,N-dimethylsulfuric diamide

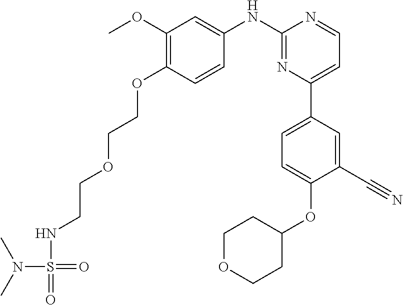 [SMILES: COc1cc(Nc2nccc(-c3ccc(OC4CCOCC4)c(C#N)c3)n2)ccc1OCCOCCNS(=O)(=O)N(C)C]

The procedure used for the preparation of Example Compound 27 was used to prepare the title compound from 5-[2-[[4-[2-(2-aminoethoxy)ethoxy]-3-methoxy-phenyl]amino]pyrimidin-4-yl]-2-tetrahydropyran-4-yloxy-benzonitrile and N,N-dimethyl-methanesulfonamide. ¹H NMR (DMSO-d₆) δ 9.56 (br s, 1H), 8.56 (d, 1H), 8.52 (d, 1H), 8.44 (dd, 1H), 7.65 (br s, 1H), 7.55 (d, 1H), 7.43 (d, 1H), 7.26 (t, 1H), 7.20 (dd, 1H), 7.93 (d, 1H), 4.95 (sept, 1H), 4.08-4.02 (m, 2H), 3.92-3.83 (m, 2H), 3.81 (s, 3H), 3.75-3.70 (m, 2H), 3.60-3.50 (m, 4H), 3.08 (q, 2H), 2.66 (s, 6H), 2.08-2.10 (m, 2H), 1.75-1.63 (m, 2H); LC-MS [M+H]⁺ 613.2438.

### Example Compound 27

### N-(2-{2-[4-({4-[3-Cyano-4-(tetrahydro-2H-pyran-4-yloxy)phenyl]pyrimidin-2-yl}amino)-2-methoxyphenoxy]ethoxy}ethyl)-4-methylpiperazine-1-sulfonamide

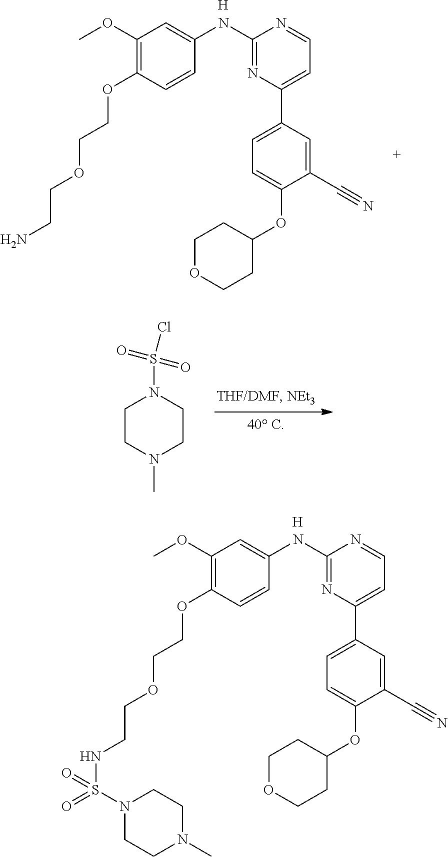

A solution of 5-[2-({4-[2-(2-aminoethoxy)ethoxy]-3-methoxyphenyl}amino) pyrimidin-4-yl]-2-(tetrahydro-2H-pyran-4-yloxy)benzonitrile (24 mg, 0.048 mmol) and Et₃N (0.25 mL) in THF (2 mL) was treated with 4-methylpiperazine-1-sulfonyl chloride hydrochloride (14.1 mg, 0.06 mmol) and stirred o/n. Et₃N (0.25 mL), DMF (0.5 mL) and 4-methylpiperazine-1-sulfonyl chloride hydrochloride (27 mg, 0.11 mmol) were added and the reaction stirred at rt for 2 h, and heated to 40° C. o/n. The reaction was concentrated onto Celite® and purified by RP-MPLC (C₁₈, MeOH/H₂O, 0-100% w/0.1% TFA) to provide the title compound. ¹H NMR (DMSO-d₆) δ 9.73 (br s, 1H), 9.57 (s, 1H), 8.56 (d, 1H), 8.53 (d, 1H), 8.43 (dd, 1H), 7.74 (t, 1H), 7.68 (br s, 1H), 7.54 (d, 1H), 7.44 (d, 1H), 7.20 (d, 1H), 6.93 (d, 1H), 4.95 (sept, 1H), 4.10-4.02 (m, 2H), 3.92-3.83 (m, 2H), 3.82 (s, 3H), 3.78-3.71 (m, 2H), 3.51 (br d, 2H), 3.60-3.51 (m, 4H), 3.48 (br d, 2H), 3.18-3.00 (m, 4H), 3.00-2.86 (m, 2H), 2.82 (br s, 3H), 2.10-1.98 (m, 2H), 1.74-1.63 (m, 2H); LC-MS [M+H]⁺ 668.2851.

### Example Compound 28

### 5-[2-({3-Methoxy-4-[3-(morpholin-4-ylsulfonyl)propoxy]phenyl}amino)pyrimidin-4-yl]-2-(tetrahydro-2H-pyran-4-yloxy)benzonitrile

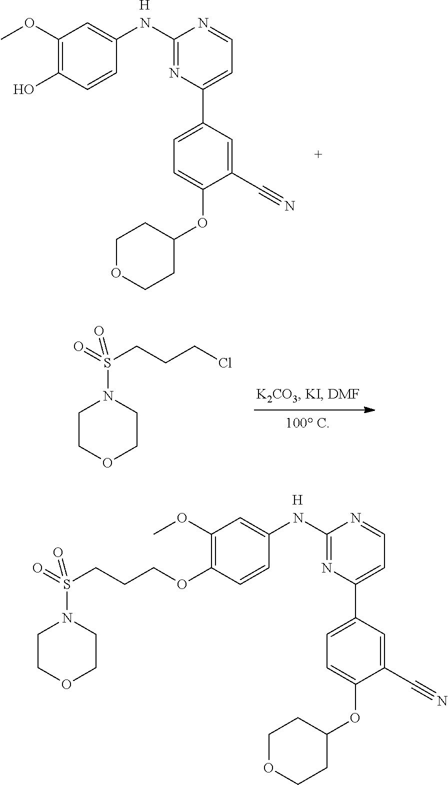

A solution of 5-{2-[(4-hydroxy-3-methoxyphenyl)amino]pyrimidin-4-yl}-2-(tetrahydro-2H-pyran-4-yloxy)benzonitrile (33 mg, 0.078 mmol), K₂CO₃ (13 mg, 0.094 mmol), KI (catalytic) and 4-(3-chloropropylsulfonyl)morpholine (20 mg, 0.088 mmol) in DMF (2 mL) was stirred at rt for 2 h, and heated to 100° C. for a total of 8 h. The reaction was diluted with EtOAc, washed with brine, dried (MgSO₄), filtered and concentrated. Purification by RP-MPLC (C₁₈, MeOH/H₂O, 0-100% w/0.1% TFA) provided the title compound. ¹H NMR (DMSO-d₆) δ 9.59 (s, 1H), 8.56 (d, 1H), 8.53 (d, 1H), 8.44 (dd, 1H), 7.68 (br s, 1H), 7.55 (d, 1H), 7.44 (d, 1H), 7.21 (d, 1H), 6.94 (d, 1H), 4.95 (sept, 1H), 4.04 (t, 2H), 3.92-3.82 (m, 2H), 3.82 (s, 3H), 3.67-3.62 (m, 4H), 3.56 (ddd, 2H), 3.28-3.21 (m, 2H), 3.20-3.15 (m, 4H), 2.14-1.98 (m, 4H), 1.74-1.62 (m, 2H); LC-MS [M+H]⁺ 610.2327.

### Example Compound 29

### N-(2-{2-[4-({4-[3-Cyano-4-(tetrahydro-2H-pyran-4-yloxy)phenyl]pyrimidin-2-yl}amino)-2-methoxyphenoxy]ethoxy}ethyl)morpholine-4-sulfonamide

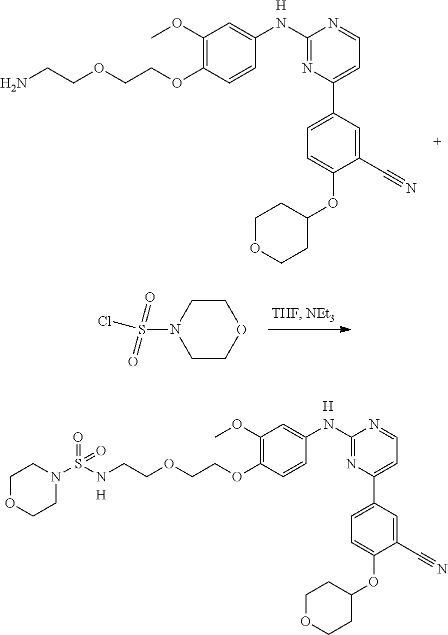

A solution of 5-[2-({4-[2-(2-aminoethoxy)ethoxy]-3-methoxyphenyl}amino)pyrimidin-4-yl]-2-(tetrahydro-2H-pyran-4-yloxy)benzonitrile (18 mg, 0.037 mmol), Et₃N (0.25 mL) and morpholine-4-sulfonyl chloride (7 μL) in THF (2 mL) was stirred at rt for 2 h. The reaction was heated to 55° C. o/n. The reaction was concentrated onto Celite® and purified by RP-MPLC (C₁₈, MeOH/H₂O, 0-100%, w/0.1% TFA) to provide the title compound. 1H NMR (DMSO-d₆) δ 9.56 (s, 1H), 8.56 (d, 1H), 8.52 (dd, 1H), 8.44 (dd, 1H), 7.66 (br s, 1H), 7.54 (d, 1H), 7.48-7.40 (m, 2H), 7.20 (d, 1H), 6.92 (d, 1H), 4.95 (sept, 1H), 4.08-4.02 (m, 2H), 3.92-3.3 (m, 2H), 3.81 (s, 3H), 3.75-3.70 (m, 2H), 3.63-3.57 (m, 5H), 3.57-3.50 (m, 3H), 3.10 (q, 2H), 3.04-2.97 (m, 4H), 2.10-1.98 (m, 2H), 1.62-1.74 (m, 2H); LC-MS [M+H]⁺ 655.2525.

### Example Compound 30

### 5-(2-{[4-(2-Aminoethoxy)-3-methoxyphenyl]amino}pyrimidin-4-yl)-2-(tetrahydro-2H-pyran-4-yloxy)benzonitrile

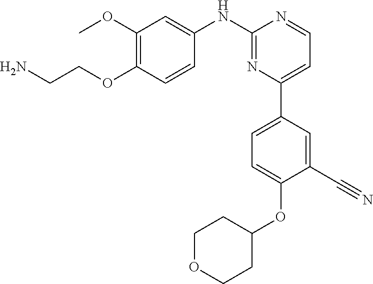 [SMILES: COc1cc(Nc2nccc(-c3ccc(OC4CCOCC4)c(C#N)c3)n2)ccc1OCCN]

Standard Method E, BOC Deprotection was used to prepare the title compound from tert-butyl N-[2-[4-[[4-(3-cyano-4-tetrahydropyran-4-yloxy-phenyl)pyrimidin-2-yl]amino]-2-methoxy-phenoxy]ethyl]carbamate. ¹H NMR (DMSO-d₆) δ 9.64 (s, 1H), 8.57 (d, 1H), 8.54 (d, 1H), 8.44 (dd, 1H), 7.96 (br s, 3H), 7.76 (s, 1H), 7.54 (d, 1H), 7.46 (d, 1H), 7.22 (d, 1H), 7.01 (d, 1H), 4.96 (sept, 1H), 4.10 (t, 2H), 3.92-3.80 (m, 2H), 3.85 (s, 3H), 3.56 (ddd, 2H), 3.22-3.12 (m, 2H), 2.10-1.98 (m, 2H), 1.75-1.62 (m, 2H); LC-MS [M+H]⁺ 462.2132.

### Example Compound 31

### 5-[2-({3-Methoxy-4-[3-(morpholin-4-yl)propoxy]phenyl}amino)pyrimidin-4-yl]-2-(tetrahydro-2H-pyran-4-yloxy)benzonitrile

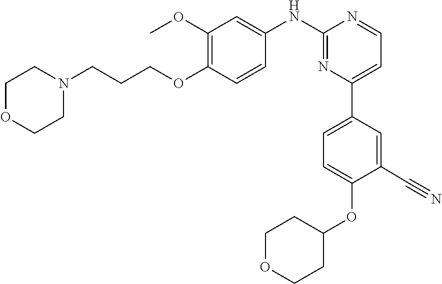 [SMILES: COc1cc(Nc2nccc(-c3ccc(OC4CCOCC4)c(C#N)c3)n2)ccc1OCCCN1CCOCC1]

The procedure used in the preparation of Intermediate I-11 was used to prepare the title compound from 5-(2-chloropyrimidin-4-yl)-2-tetrahydropyran-4-yloxy-benzonitrile and 3-methoxy-4-(3-morpholinopropoxy)aniline. ¹H NMR (DMSO-d₆) δ 9.60 (s, 1H), 8.56 (d, 1H), 8.53 (d, 1H), 8.43 (dd, 1H), 7.71 (br s, 1H), 7.54 (d, 1H), 7.44 (d, 1H), 7.21 (d, 1H), 6.95 (d, 1H), 4.95 (sept, 1H), 4.08-3.95 (m, 4H), 3.92-3.78 (m, 2H) 3.83 (s, 3H), 3.65 (t, 2H), 3.60-3.48 (m, 4H), 3.36-3.25 (m, 2H), 3.18-3.05 (m, 2H), 2.18-1.99 (m, 4H), 1.75-1.62 (m, 2H); LC-MS [M+H]⁺ 546.2714.

### Example Compound 32

### 5-[2-({3-[2-(2-Aminoethoxy)ethoxy]-4-methoxyphenyl}amino)pyrimidin-4-yl]-2-(tetrahydro-2H-pyran-4-yloxy)benzonitrile

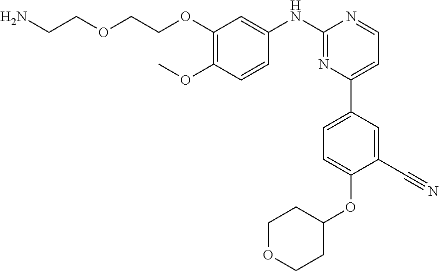 [SMILES: COc1ccc(Nc2nccc(-c3ccc(OC4CCOCC4)c(C#N)c3)n2)cc1OCCOCCN]

Standard Method E, BOC Deprotection was used to prepare the title compound from tert-butyl N-[2-[2-[5-[[4-(3-cyano-4-tetrahydropyran-4-yloxy-phenyl)pyrimidin-2-yl]amino]-2-methoxy-phenoxy]ethoxy]ethyl]carbamate. ¹H NMR (DMSO-d₆) δ 9.54 (s, 1H), 8.54 (d, 1H), 8.52 (d, 1H), 8.43 (dd, 1H), 7.78 (br s, 3H), 7.60 (br s, 1H), 7.54 (d, 1H), 7.43 (d, 1H), 7.27 (dd, 1H), 6.94 (d, 1H), 4.95 (sept, 1H), 4.18-4.10 (m, 2H), 3.90-3.80 (m, 4H), 3.75 (s, 3H), 3.72-3.68 (m, 2H), 3.56 (ddd, 2H), 3.08-2.98 (m, 2H), 2.10-1.98 (m, 2H), 1.74-1.62 (m, 2H); LC-MS [M+H]⁺ 506.2402.

### Example Compound 314

### 3-{2-[(3,4-Dimethoxyphenyl)amino]quinazolin-4-yl}benzonitrile

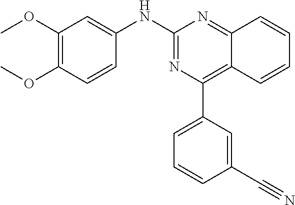 [SMILES: COc1ccc(Nc2nc(-c3cccc(C#N)c3)c3ccccc3n2)cc1OC]

A solution of 3-(2-chloroquinazolin-4-yl)benzonitrile (1.03 mmol), 3,4-dimethoxyaniline (169 mg, 1.10 mmol), catalytic conc. HCl (2 drops) in i-PrOH was heated to reflux o/n. The reaction was concentrated and purified by RP-MPLC (C₁₈, MeOH/H₂O, 0-100%, w/0.1% TFA) to provide the title compound. ¹H NMR (DMSO-d₆) 9.85 (s, 1H), 8.28-8.24 (m, 1H), 8.14-8.08 (m, 2H), 7.90-7.80 (m, 3H), 7.79-7.70 (m, 2H), 7.48-7.38 (m, 1H), 7.38-7.30 (m, 1H), 6.93 (d, 1H), 3.80 (s, 3H), 3.74 (s, 3H); TOF LC-MS [M+H]⁺ 383.1501.

### Example Compound 334

### 1-(3-{[4-(3-Cyano-4-methoxyphenyl)pyrimidin-2-yl]amino}-5-methoxyphenyl)-3-cyclopentylurea

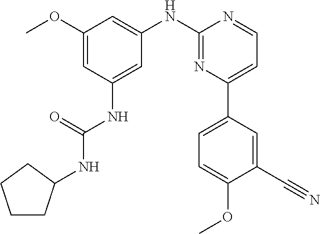 [SMILES: COc1cc(NC(=O)NC2CCCC2)cc(Nc2nccc(-c3ccc(OC)c(C#N)c3)n2)c1]

A solution of 5-[2-[(3-amino-5-methoxy-phenyl)amino]pyrimidin-4-yl]-2-methoxy-benzonitrile (36 mg, 0.10 mmol) and Et₃N (0.25 mL) in THF (2 mL) and DMF (0.5 mL) was treated with excess isocyanatocyclopentane and stirred o/n. The reaction was concentrated and purified by MPLC (SiO₂, EtOAc/Hexanes, 0-100%). A second purification by MPLC (CH₂Cl₂/MeOH, 0-20%, w/0.1% NH₄OH) provided the title compound. ¹H NMR (DMSO-d₆) δ 9.62 (s, 1H), 8.65-8.55 (m, 2H), 8.54 (d, 1H), 8.25 (s, 1H), 7.48 (d, 1H), 7.42 (d, 1H), 7.35 (s, 1H), 7.12 (s, 1H), 6.81 (s, 1H), 6.14 (d, 1H), 4.01 (s, 3H), 3.97 (q, 1H), 3.73 (s, 3H), 1.90-1.80 (m, 2H), 1.70-1.58 (m, 2H), 1.58-1.47 (m, 2H), 1.42-1.30 (m, 2H). TOF LC-MS [M+H]⁺ 459.2143.

### Example Compound 351

### 3-{[4-(3-Cyano-4-methoxyphenyl)pyrimidin-2-yl]amino}-N-cyclopentyl-5-methoxybenzamide

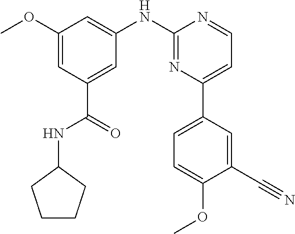 [SMILES: COc1cc(Nc2nccc(-c3ccc(OC)c(C#N)c3)n2)cc(C(=O)NC2CCCC2)c1]

A solution of 3-[[4-(3-cyano-4-methoxy-phenyl)pyrimidin-2-yl]amino]-5-methoxy-benzoic acid (62 mg, 0.16 mmol) and Et₃N (0.25 mL) in THF (2 mL) was treated with ethyl chloroformate (0.02 mL) and stirred o/n. Additional ethylchloroformate (0.12 mL) was added, at which point a vigorous reaction was observed. THF (1 mL) and DMF (0.5 mL) were added, followed by cyclopentylamine (0.2 mL). The reaction was stirred for 1 h., concentrated and purified by MPLC (SiO₂, EtOAc/Hexanes, 0-100%) to provide the title compound. ¹H NMR (DMSO-d₆) 9.81 (s, 1H), 8.60-8.55 (m, 2H), 8.53 (dd, 1H), 8.21 (d, 1H), 7.83 (t, 1H), 7.69 (t, 1H), 7.52 (d, 1H), 7.42 (d, 1H), 6.99 (dd, 1H), 4.23 (sextet, 1H), 4.02 (s, 3H), 3.83 (s, 3H), 1.95-1.82 (m, 2H), 1.75-1.63 (m, 2H), 1.58-1.46 (m, 4H). TOF LC-MS [M+H]⁺ 444.2038.

### Example Compound 393

### N-(3-{[(3-{[4-(3-Cyano-4-methoxyphenyl)pyrimidin-2-yl]amino}-5-methoxyphenyl)carbamoyl]amino}propyl)acetamide

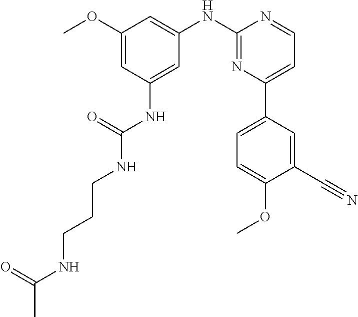 [SMILES: COc1cc(NC(=O)NCCCNC(C)=O)cc(Nc2nccc(-c3ccc(OC)c(C#N)c3)n2)c1]

A solution of 1-(3-aminopropyl)-3-(3-{[4-(3-cyano-4-methoxyphenyl)pyrimidin-2-yl]amino}-5-methoxyphenyl)urea (73.4 mg, 0.13 mmol) and Et₃N (0.25 mL) in THF (2 mL) was treated with acetyl chloride (0.02 mL, 0.28 mmol) and stirred at rt for 2 h. The reaction was concentrated and purification by MPLC (SiO₂, EtOAc/Hexanes, 0-100% then 100% EtOAc to 100% 1:1 CH₂Cl₂/MeOH) provided the title compound. TOF LC-MS [M+H]⁺ 490.2197.

### Preparation of Example 457

### N-[2-Cyano-4-[2-[[4-(2-diethylaminoethyl)phenyl]amino]pyrimidin-4-yl]phenyl]-2-methyl-propanamide

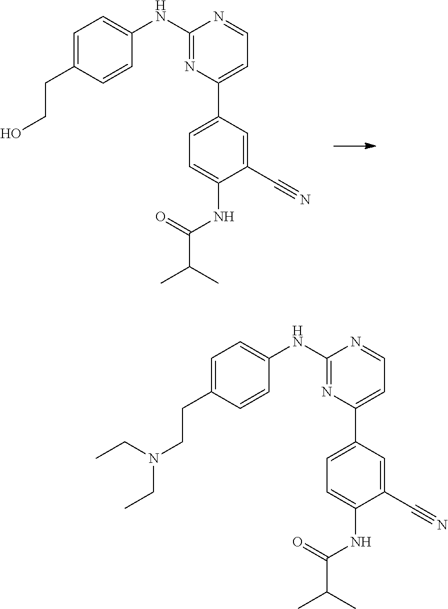 [SMILES: CC(C)C(=O)Nc1ccc(-c2ccnc(Nc3ccc(CCO)cc3)n2)cc1C#N ; CCN(CC)CCc1ccc(Nc2nccc(-c3ccc(NC(=O)C(C)C)c(C#N)c3)n2)cc1]

To a solution of N-[2-cyano-4-[2-[[4-(2-hydroxyethyl)phenyl]amino]pyrimidin-4-yl]phenyl]-2-methyl-propanamide in CH₂Cl₂ (10 mL) was added DIPEA (0.2 mL), methylsulfonyl chloride (0.04 mL) at 0° C. and the reaction mixture was stirred at rt for 1 h. The mixture was added Et₂NH (0.5 mL), and concentrated under the reduced pressure to remove CH₂Cl₂. The residue was diluted with DMF (5 mL), and the solution was stirred at 80° C. for 5 h. The reaction mixture was concentrated under the reduced pressure, and the crude product was purified by column chromatography (SiO₂, MeOH 020% in CH₂Cl₂ with 0.1% NH₄OH). ¹H NMR (DMSO-d₆) δ 10.3 (s, 1H), 9.78 (s, 1H), 9.32 (br s, 1H, TFA), 8.60-8.57 (m, 2H), 8.47-8.44 (m, 1H), 7.80-7.77 (m, 3H), 7.51 (d, 1H), 7.28 (d, 2H), 3.30-3.16 (m, 6H), 2.96-2.90 (m, 2H), 2.77-2.70 (m, 1H), 1.23 (t, 6H), 1.16 (d, 6H). TOF LC-MS [M+H]⁺ 457.2790.

### Preparation of Example 461

### 3-[2-Cyano-4-[2-[(4-morpholinophenyl)amino]pyrimidin-4-yl]phenoxy]-N-(2-dimethylaminoethyl)-2,2-dimethyl-propanamide

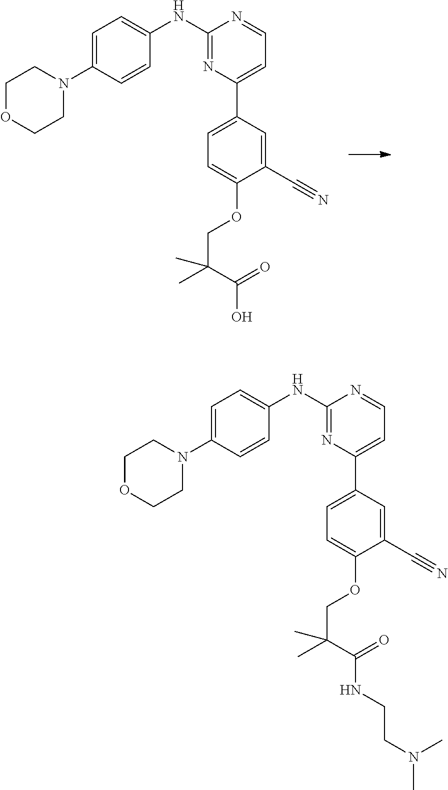 [SMILES: CC(C)(COc1ccc(-c2ccnc(Nc3ccc(N4CCOCC4)cc3)n2)cc1C#N)C(=O)O ; CN(C)CCNC(=O)C(C)(C)COc1ccc(-c2ccnc(Nc3ccc(N4CCOCC4)cc3)n2)cc1C#N]

To a solution of 3-[2-cyano-4-[2-[(4-morpholinophenyl)amino]pyrimidin-4-yl]phenoxy]-2,2-dimethyl-propanoic acid (0.100 g, 0.21 mmol) in DMF (3 mL) was added N′,N′-dimethylethane-1,2-diamine (0.05 mL), HBTU (0.114 g, 3.0 mmol), and DIPEA (0.1 mL), and the mixture was stirred at rt for 15 h. The mixture was concentrated, and purified by preparative HPLC to give the title compound. ¹H NMR (DMSO-d₆) 9.55 (s, 1H), 8.52-8.45 ((s, 3H), 7.67 (d, 2H), 7.44 (d, 1H), 7.41 (d, 1H), 7.00 (apparent d, 2H), 4.27 (s, 2H), 3.78-3.76 (m, 4H), 3.55 (t, 2H), 3.14 (s, 6H), 3.14-3.05 (m, 4H), 2.59 (t, 2H), 1.41 (s, 6H). TOF LC-MS [M+H]⁺ 544.2899.

### Preparation of Example 467

### N-[2-Cyano-4-[2-[[4-(2-hydroxyethyl)phenyl]amino]pyrimidin-4-yl]phenyl]-2-methyl-propanamide

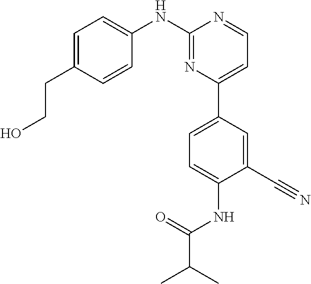 [SMILES: CC(C)C(=O)Nc1ccc(-c2ccnc(Nc3ccc(CCO)cc3)n2)cc1C#N]

This intermediate was prepared by the procedure described for the preparation of Intermediate I-11 using a Buchwald coupling reaction. ¹H NMR (DMSO-d₆) δ 10.3 (s, 1H), 9.67 (s, 1H), 8.58-8.56 (m, 2H), 8.47-8.44 (m, 1H), 7.78 (d, 1H), 7.70 (d, 2H), 7.48 (d, 1H), 7.16 (d, 2H), 4.64 (t, 1H), 3.61-3.56 (m, 2H), 2.77-2.67 (m, 3H), 1.16 (d, 6H). TOF LC-MS [M+H]402.1771.

### Preparation of Example 476

### 2-(1-Isopropylazetidin-3-yl)oxy-5-[2-[(4-morpholinophenyl)amino]pyrimidin-4-yl]benzonitrile

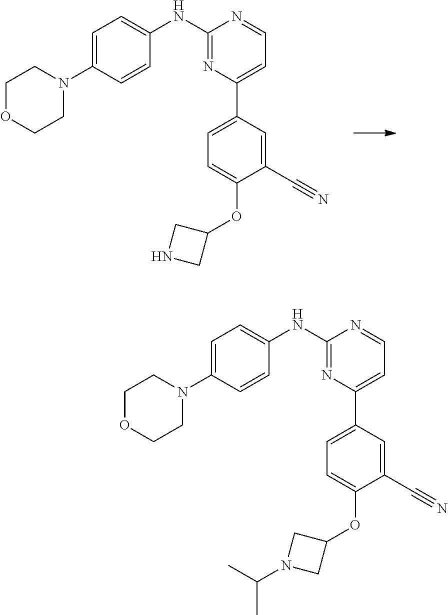 [SMILES: N#Cc1cc(-c2ccnc(Nc3ccc(N4CCOCC4)cc3)n2)ccc1OC1CNC1 ; CC(C)N1CC(Oc2ccc(-c3ccnc(Nc4ccc(N5CCOCC5)cc4)n3)cc2C#N)C1]

To a solution of 2-(azetidin-3-yloxy)-5-[2-[(4-morpholinophenyl)amino]pyrimidin-4-yl]benzonitrile (0.100 g, 0.23 mmol) in DMF (5 mL) was added 2-iodopropanol (0.2 mL) and K₂CO₃ (0.15 g), and the mixture was stirred at 65° C. for 15 h. The mixture was added H₂O (10 mL), extracted with i-PrOH/CHCl₃ (1:3), dried (MgSO₄) and concentrated under reduced pressure. The crude product was purified by reverse phase column chromatography (C₁₈, CH₃CN 95.0% in H₂O with 0.1% TFA) and following preparative HPLC to give the title product. ¹H NMR (CDCl₃) δ 8.44 (d, 1H), 8.32 (d, 1H), 8.22-8.19 (m, 1H), 7.55-7.52 (m, 2H), 7.05 (s, 1H), 7.01 (d, 1H), 6.98-6.95 (m, 2H), 6.84 (d, 1H), 4.94-4.91 (m, 1H), 3.97-3.92 (m, 2H), 3.90-3.87 (m, 4H), 3.24-3.18 (m, 2H), 3.16-3.13 (m, 4H), 1.01 (d, 6H). TOF LC-MS [M+H]⁺ 471.2513.

### Example Compound 489

### 5-[2-[[4-[(2-5-[2-[[4-(Aminomethyl)phenyl]amino]pyrimidin-4-yl]-2-tetrahydropyran-4-yloxy-benzonitrile

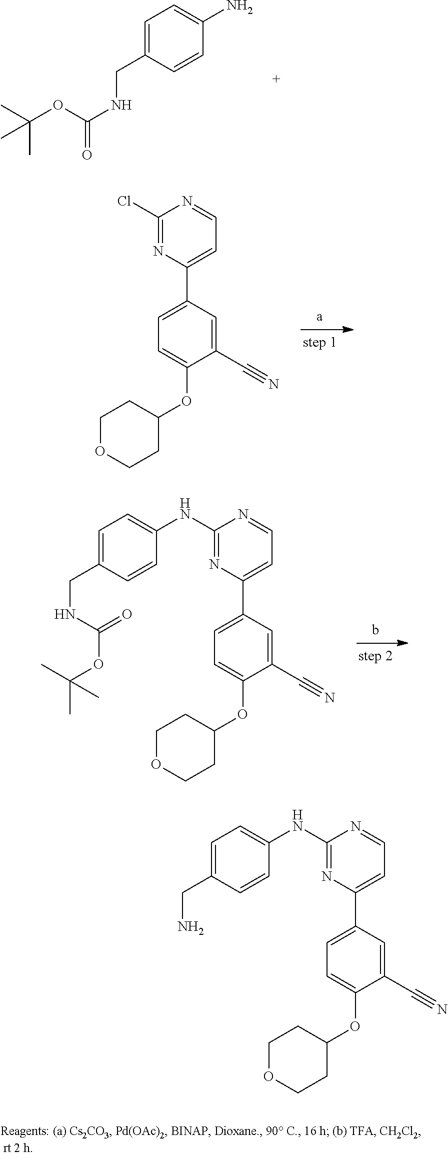

Step 1. tert-Butyl N-[[4-[[4-(3-cyano-4-tetrahydropyran-4-yloxy-phenyl)pyrimidin-2-yl]amino]phenyl]methyl]carbamate: The title compound was prepared from 5-(2-chloropyrimidin-4-yl)-2-tetrahydropyran-4-yloxy-benzonitrile (0.60 g, 1.90 mmol) and tert-butyl N-[(4-aminophenyl)methyl]carbamate (0.633, 2.85 mmol) according to procedure used for Intermediate I-11 (0.45 g, 47%). ¹H NMR (DMSO-d₆) δ 9.67 (s, 1H), 8.54 (d, 1H), 8.53 (d, 1H), 8.44 (dd, 1H), 7.72 (d, 2H), 7.56 (d, 1H), 7.47 (d, 1H), 7.36 (t, 1H), 7.17 (d, 2H), 4.98-4.92 (m, 1H), 4.07 (d, 2H), 3.91-3.84 (m, 2H), 3.59-3.52 (m, 2H), 2.08-2.00 (m, 2H), 1.95-1.84 (m, 2H), 1.74-1.63 (m, 2H), 1.40 (s, 9H).

Step 2. 5-[2-[[4-[(2-5-[2-[[4-(Aminomethyl)phenyl]amino]pyrimidin-4-yl]-2-tetrahydropyran-4-yloxy-benzonitrile: To a solution of tert-butyl N-[[4-[[4-(3-cyano-4-tetrahydropyran-4-yloxy-phenyl)pyrimidin-2-yl]amino]phenyl]methyl]carbamate (0.02 g, 0.90 mmol) in CH₂Cl₂ (1.5 mL) was added TFA (1.5 mL). The reaction mixture was stirred at rt. for 4 h. The solvent was evaporated. Purification by RP HPLC afforded the title compound as the trifluoroacetate salt (0.011 g, 53%). ¹H NMR (DMSO-d₆) δ 9.85 (s, 1H), 8.58 (d, 1H), 8.55 (d, 1H), 8.45 (dd, 1H), 8.05 (br s, 2H), 7.85 (d, 2H), 7.55 (d, 1H), 7.51 (d, 1H), 7.40 (d, 2H), 4.98-4.94 (m, 1H), 4.01-3.96 (m, 2H), 3.90-3.85 (m, 2H), 3.59-3.53 (m, 2H), 2.08-2.00 (m, 2H), 1.73-1.65 (m, 2H). LC-MS [M+H]⁺ 402.1927.

### Example Compound 491

### 5-[2-[[4-[(2-Methoxyethylamino)methyl]phenyl]amino]pyrimidin-4-yl]-2-tetrahydropyran-4-yloxy-benzonitrile

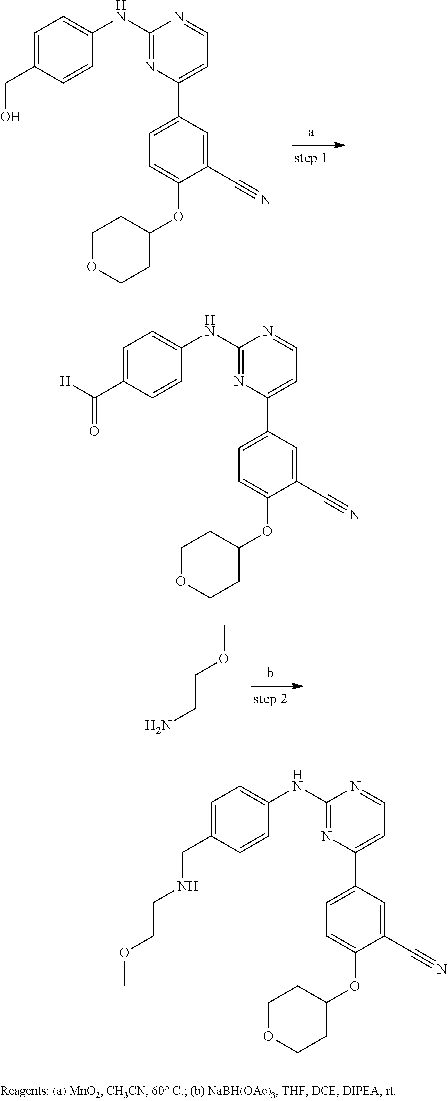

Step 1. 5-[2-[(4-Formylphenyl)amino]pyrimidin-4-yl]-2-tetrahydropyran-4-yloxy-benzonitrile: To a mixture of 5-[2-[[4-(hydroxymethyl)phenyl]amino]pyrimidin-4-yl]-2-tetrahydropyran-4-yloxy-benzonitrile (0.20 g, 0.50 mmol) in CH₃CN was added MnO₂ (0.22 g, 2.50 mmol). The reaction mixture was placed in an oil bath at 60° C. and stirred o/n. The reaction mixture was filtered hot through Celite®, washed with hot CH₃CN (5×50 mL) and the solvent evaporated under reduced pressure to afford the title compound (0.16 g, 80%). ¹H NMR (DMSO-d₆) δ 10.3 (s, 1H), 9.86 (s, 1H), 8.66 (d, 1H), 8.59 (d, 1H), 8.50 (dd, 1H), 8.06 (d, 2H), 7.88 (d, 2H), 7.63 (d, 1H), 7.58 (d, 1H), 4.99-4.92 (m, 1H), 3.91-3.85 (m, 2H), 3.59-3.53 (dd, 2H), 2.08-2.01 (m, 2H), 1.95-1.84 (m, 2H), 1.76-1.66 (m, 2H). LC-MS [M+H]⁺ 401.

Step 2. 5-[2-[[4-[(2-Methoxyethylamino)methyl]phenyl]amino]pyrimidin-4-yl]-2-tetrahydropyran-4-yloxy-benzonitrile: To a mixture of 5-[2-[[4-(hydroxymethyl)phenyl]amino]pyrimidin-4-yl]-2-tetrahydropyran-4-yloxy-benzonitrile (0.050 g, 0.125 mmol) and 2-methoxyethanamine (0.016 mL, 0.187 mmol) in THF/DCE (2:1, 5.0 mL) was added DIPEA (0.025 mL, 0.144 mmol) and sodium triacetoxyborohydride (0.040 g, 0.187 mmol). The reaction mixture was stirred o/n at rt. Saturated aq. NaHCO₃ (5.0 mL) was added, the reaction mixture was stirred for 15 min and the layers separated. The aqueous layer was extracted with EtOAc (3×5.0 mL), the organic layers combined, dried over sodium sulfate, filtered and evaporated. Purification by RP HPLC followed by recrystallization/precipitation from Hexanes/EtOAc afforded the title compound as the trifluoroacetate salt (0.013 g, 18%). ¹H NMR (DMSO-d₆) δ 9.89 (s, 1H), 8.80 (br s, 1H), 8.59 (d, 1H), 8.55 (d, 1H), 8.45 (dd, 1H), 7.77 (d, 2H), 7.55 (d, 1H), 7.52 (d, 1H), 7.43 (d, 2H), 4.99-4.93 (m, 1H), 4.11 (s, 2H), 3.90-3.85 (m, 2H), 3.59-3.53 (m, 4H), 3.35 (s, 3H), 3.09 (br s, 2H), 2.08-2.02 (m, 2H), 1.73-1.65 (m, 2H). LC-MS [M+H]⁺ 460.2345.

### Example Compound 498

### 5-[2-[[4-[(2-5-[2-[[4-(Aminomethyl)phenyl]amino]pyrimidin-4-yl]-2-tetrahydropyran-4-yloxy-benzonitrile

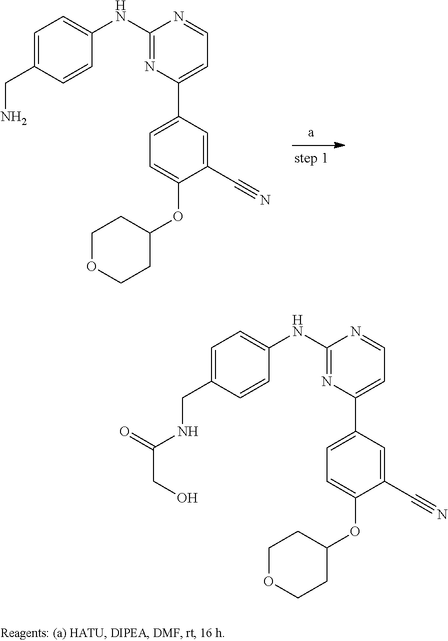 [SMILES: N#Cc1cc(-c2ccnc(Nc3ccc(CN)cc3)n2)ccc1OC1CCOCC1 ; N#Cc1cc(-c2ccnc(Nc3ccc(CNC(=O)CO)cc3)n2)ccc1OC1CCOCC1]

Step 1. N-[[4-[[4-(3-Cyano-4-tetrahydropyran-4-yloxy-phenyl)pyrimidin-2-yl]amino]phenyl]methyl]-2-hydroxy-acetamide: The title compound was prepared from 5-[2-[[4-[(2-5-[2-[[4-(aminomethyl)phenyl]amino]pyrimidin-4-yl]-2-tetrahydropyran-4-yloxy-benzonitrile (0.040 g, 0.097 mmol) and glycolic acid (0.010 g, 0.125 mmol) according to the Standard Method H; HATU Coupling (0.012 g, 21%). ¹H NMR (DMSO-d₆) δ 9.68 (s, 1H), 8.55 (s, 1H), 8.53 (d, 1H), 8.45 (dd, 1H), 8.22 (t, 1H), 7.73 (d, 2H), 7.57 (d, 1H), 7.46 (d, 1H), 7.22 (d, 2H), 4.98-4.91 (m, 1H), 4.26 (d, 2H), 3.90-3.85 (m, 2H), 3.85 (s, 2H), 3.58-3.53 (m, 2H), 2.08-2.01 (m, 2H), 1.72-1.65 (m, 2H). LC-MS [M+H]⁺ 460.1962.

### Example Compound 500

### 5-[2-[[4-[(3-Hydroxyazetidin-1-yl)methyl]phenyl]amino]pyrimidin-4-yl]-2-tetrahydropyran-4-yloxy-benzonitrile

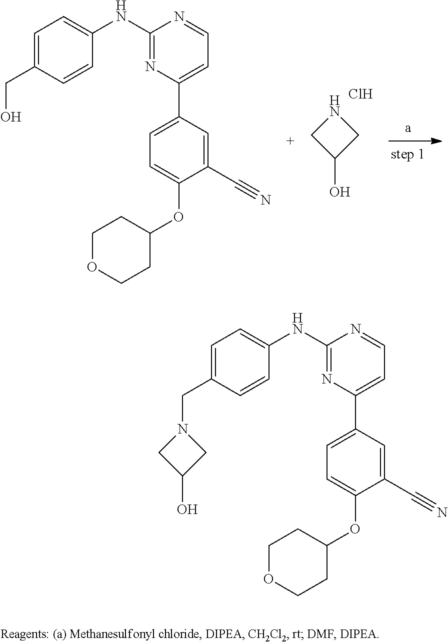 [SMILES: N#Cc1cc(-c2ccnc(Nc3ccc(CO)cc3)n2)ccc1OC1CCOCC1 ; OC1CNC1 ; Cl ; N#Cc1cc(-c2ccnc(Nc3ccc(CN4CC(O)C4)cc3)n2)ccc1OC1CCOCC1]

Step 1. 5-[2-[[4-[(3-Hydroxyazetidin-1-yl)methyl]phenyl]amino]pyrimidin-4-yl]-2-tetrahydropyran-4-yloxy-benzonitrile: To a mixture of 5-[2-[[4-(hydroxymethyl)phenyl]amino]pyrimidin-4-yl]-2-tetrahydropyran-4-yloxy-benzonitrile (0.045 g, 0.111 mmol) in CH₂Cl₂ was added methanesulfonyl chloride (0.017 mL, 0.222 mmol) and DIPEA (0.040 mL, 0.222 mmol). The reaction mixture was stirred for 1 h at rt. The solvent was evaporated under reduced pressure, DMF (2 mL), DIPEA (0.040 mL, 0.222 mmol) and azetidin-3-ol hydrochloride (0.025 g, 0.222 mmol) were added and the reaction mixture stirred for 2 h at rt. Purification by reverse phase HPLC afforded the title compound as the trifluoroacetate salt (0.007 g, 10%). ¹H NMR (DMSO-d₆) δ 9.92 (s, 1H), 9.71 (br s, 1H), 8.59 (d, 1H), 8.55 (d, 1H), 8.45 (dd, 1H), 7.88 (d, 2H), 7.54 (dd, 2H), 7.47-7.44 (m, 1H), 7.42 (d, 1H), 5.09 (t, 1H), 4.98-4.90 (m, 1H), 4.45 (d, 2H), 3.90-3.85 (m, 2H), 3.58-3.52 (m, 2H), 2.08-2.00 (m, 2H), 1.73-1.65 (m, 2H). LC-MS [M+H]⁺ 458.2168.

### Example Compound 501

### 5-[2-[[4-(Hydroxymethyl)-3-methoxy-phenyl]amino]pyrimidin-4-yl]-2-tetrahydropyran-4-yloxy-benzonitrile

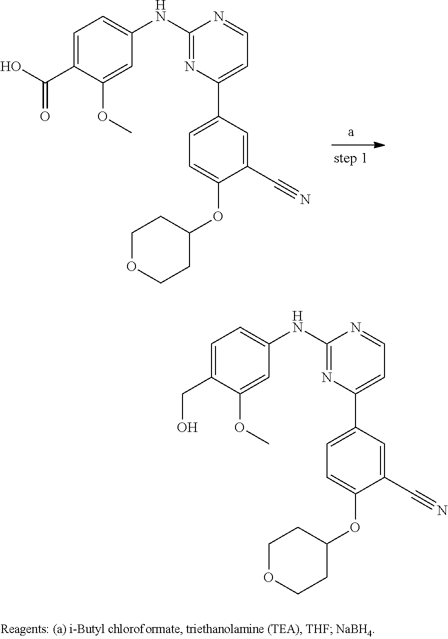 [SMILES: COc1cc(Nc2nccc(-c3ccc(OC4CCOCC4)c(C#N)c3)n2)ccc1C(=O)O ; COc1cc(Nc2nccc(-c3ccc(OC4CCOCC4)c(C#N)c3)n2)ccc1CO]

Step 1. 5-[2-[[4-(Hydroxymethyl)-3-methoxy-phenyl]amino]pyrimidin-4-yl]-2-tetrahydropyran-4-yloxy-benzonitrile: To a mixture of 4-[[4-(3-cyano-4-tetrahydropyran-4-yloxy-phenyl)pyrimidin-2-yl]amino]-2-methoxy-benzoic acid (0.75 g, 1.68 mmol) in THF (30 mL) was added TEA (0.35 mL, 2.52 mmol), and the solution cooled to 0° C. i-Butyl chloroformate (0.34 g, 2.52 mmol) was added, the solution warmed to rt and stirred for 4 h. The reaction mixture was cooled to 0° C., NaBH₄ (0.255, 6.73 mmol) was added slowly and the solution allowed to warmed to rt and stirred for 2 h. H₂O and sat. aq. NaHCO₃ (10 mL) were added, the mixture stirred vigorously for 30 min and extracted with CH₂Cl₂ (2×25 mL) and EtOAc/1% MeOH (2×25 mL) and CHCl₃ (2×25 mL). The organic layers were combined, dried over sodium sulfate, filtered and evaporated. Purification by column chromatography Hexanes/EtOAc to EtOAc/EtOAc with 10% MEOH afforded the title compound (0.30 g, 41%). ¹H NMR (DMSO-d₆) δ 9.69 (s, 1H), 8.58 (d, 1H), 8.56 (d, 1H), 8.46 (dd, 1H), 7.68 (s, 1H), 7.56 (d, 1H), 7.47 (d, 1H), 7.26 (t, 2H), 4.98-4.93 (m, 1H), 4.87 (t, 1H), 4.45 (d, 1H), 3.90-3.85 (m, 2H), 3.82 (s, 3H), 3.58-3.53 (m, 2H), 2.06-1.98 (m, 2H), 1.73-1.64 (m, 2H). LC-MS [M+H]⁺ 433.1835.

### Example Compound 503

### 5-[2-[[4-(Imidazol-1-ylmethyl)phenyl]amino]pyrimidin-4-yl]-2-tetrahydropyran-4-yloxy-benzonitrile

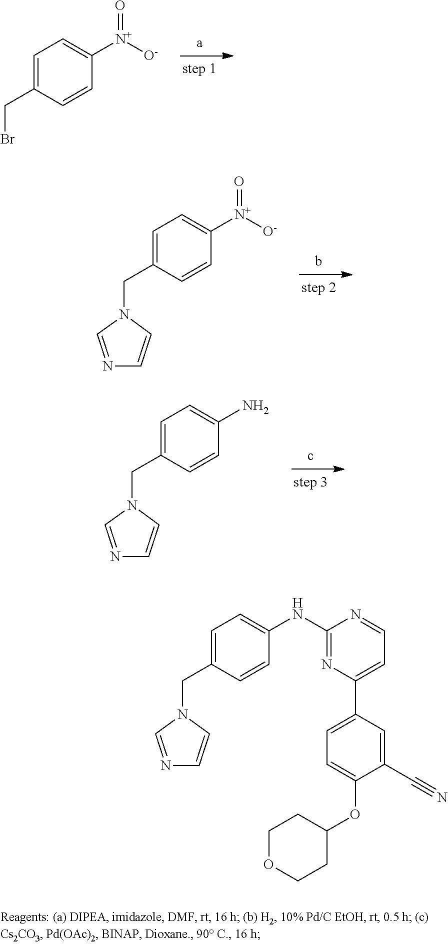 [SMILES: O=[N+]([O-])c1ccc(CBr)cc1 ; O=[N+]([O-])c1ccc(Cn2ccnc2)cc1 ; Nc1ccc(Cn2ccnc2)cc1 ; N#Cc1cc(-c2ccnc(Nc3ccc(Cn4ccnc4)cc3)n2)ccc1OC1CCOCC1]

Step 1. 1-[(4-Nitrophenyl)methyl]imidazole: 1-(Bromomethyl)-4-nitro-benzene (1.0 g, 4.6 mmol) was dissolved in DMF (2.0 mL) and added to a solution of imidazole (1.89 g, 27.7 mmol) and DIPEA (0.90 mL, 5.09 mmol) in DMF (10 mL). The reaction mixture was stirred for 16 h. The solvent was removed and H₂O and EtOAc were added. The organic layer was separated, dried over sodium sulfated and the solvent evaporated. Purification by column chromatography afforded the title compound (0.8 g, 85%). ¹H NMR (DMSO-d₆) δ 8.23 (dt, 2H), 7.80 (d, 1H), 7.46 (dt, 2H), 7.23 (t, 1H), 6.95 (t, 1H), 5.39 (s, 1H).

Step 2. 4-(Imidazol-1-ylmethyl)aniline: To a nitrogen purged solution of 1-[(4-nitrophenyl)methyl]imidazole (0.8 g, 3.98 mmol) in EtOH (10 mL) was added 10% Pd/C (0.08 g).

The reaction mixture was flushed with H₂ (g) for 5 min and stirred for 0.5 h. The reaction mixture was filtered through Celite® and concentrated under reduced pressure to afford the title compound. ¹H NMR (DMSO-d₆) δ 7.65 (s, 1H), 7.10 (t, 1H), 6.97 (dt, 2H), 6.85 (t, 1H), 6.51 (dt, 2H), 5.11 (s, 2H), 4.94 (s, 2H).

Step 3. 5-[2-[[4-(Imidazol-1-ylmethyl)phenyl]amino]pyrimidin-4-yl]-2-tetrahydropyran-4-yloxy-benzonitrile: 5-(2-Chloropyrimidin-4-yl)-2-(tetrahydro-2H-pyran-4-yloxy)benzonitrile (0.10 g, 0.31 mmol), 4-(imidazol-1-ylmethyl)aniline (0.08 g, 0.47 mmol), cesium carbonate (0.31 g, 0.95 mmol), Pd(OAc)₂ (0.10 g, 0.05 mmol) and BINAP (0.05 g, 0.08 mmol) and toluene (10 mL) were added to a flask and the reaction mixture sparged with nitrogen (3 min). The reaction mixture was placed in an oil bath at 90° C. and stirred for 14 h. The reaction was cooled to rt, H₂O (5.0 mL) and EtOAc (25 mL) were added, the aqueous layer extracted with EtOAc (3×15 mL), the organic layers combined, dried over sodium sulfate, filtered and evaporated. Purification by column chromatography (Hexanes/EtOAc to EtOAc/10% MeOH/CH₂Cl₂ with 1% NH₄OH) followed by recrystallization/precipitation from Hexanes/EtOAc afforded the title compound (0.035 g, 25%). ¹H NMR (DMSO-d₆) δ 10.1 (br s, 1H), 8.62 (d, 2H), 8.48 (s, 1H), 7.90 (s, 1H), 7.72 (d, 1H), 7.59 (t, 2H), 7.39 (d, 1H), 4.98-4.96 (m, 1H), 3.90-3.86 (m, 2H), 3.88 (s, 3H), 3.76 (s, 3H), 3.59-3.56 (m, 2H), 2.08-2.03 (m, 2H), 1.69 (m, 2H); TOF [M+H]⁺ 461.1816.

A fraction of the material (0.025 g, 0.055 mmol) was converted to the HCl salt by addition of 1N HCl and MeOH, stirring for 5 min, evaporation of the solvent and recrystallization/precipitation from Hexanes/EtOAc (0.020 g, 74%).

### Example Compound 534

### 5-[2-[[3-[2-(2-Aminoethoxy)ethoxy]-4-methoxy-phenyl]amino]pyrimidin-4-yl]-2-tetrahydropyran-4-yloxy-benzonitrile

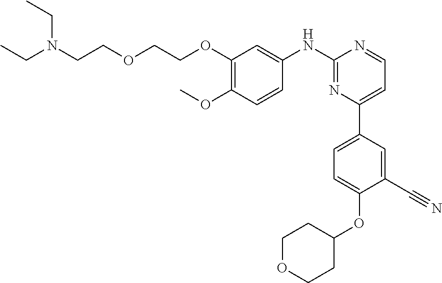 [SMILES: CCN(CC)CCOCCOc1cc(Nc2nccc(-c3ccc(OC4CCOCC4)c(C#N)c3)n2)ccc1OC]

A solution of 5-[2-({3-[2-(2-aminoethoxy)ethoxy]-4-methoxyphenyl}amino)pyrimidin-4-yl]-2-(tetrahydro-2H-pyran-4-yloxy)benzonitrile (54 mg, 0.087 mmol), NaBH₃CN (16.1 mg, 0.26 mmol) in MeOH (2 mL) was treated with acetaldehyde (0.01 mL, 0.18 mmol) and stirred o/n. The reaction was quenched with sat. NaHCO₃, extracted with EtOAc, dried (MgSO₄), filtered and concentrated. Purification by RP-MPLC (C₁₈, MeOH/H₂O, 0-100%, with 0.1% TFA) provided the title compound. ¹H NMR (DMSO-d₆) δ 9.54 (s, 1H), 9.11 (br s, 1H), 8.54 (d, 1H), 8.52 (d, 1H), 8.42 (dd, 1H), 7.62 (s, 1H), 7.54 (d, 1H), 7.43 (d, 1H), 7.25 (dd, 1H), 6.94 (d, 1H), 4.95 (sept., 1H), 4.20-4.12 (m, 2H), 3.93-3.78 (m, 6H), 3.75 (s, 3H), 3.56 (ddd, 2H), 3.31 (q, 2H), 3.25-3.09 (m, 4H), 2.10-1.98 (m, 2H), 1.75-1.62 (m, 2H), 1.17 (t, 6H); TOF LC-MS [M+H]⁺ 562.3034.

The structures and physicochemical characterization of synthesized example compounds are provided in Table 2 below. The compounds were synthesized using the methods and intermediates outlined above using commercially available starting materials that are well known in the art. IUPAC names for the compounds depicted were generated using Advanced Chemistry Development, Inc., (ACD/Labs) (Toronto, Ontario, Canada) ACD/Name IUPAC nomenclature software release 12.00, version 12.01

TABLE 2
Example Compounds

| Ex- | | | |
|---|---|---|---|
| am- | | | |
| ple | | | |
| No. | Structure | IUPAC Name | Analytical Data |
| 1 | 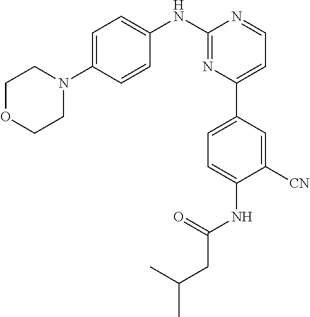 [SMILES: CC(C)CC(=O)Nc1ccc(-c2ccnc(Nc3ccc(N4CCOCC4)cc3)n2)cc1C#N] | N-[2-cyano-4-(2-{[4- (morpholin-4- yl)phenyl]amino} pyrimidin-4-yl)phenyl]- 3-methylbutanamide | ¹H NMR (CDCl₃) δ 8.45 (d, 1H), 8.44 (d, 1H), 8.33 (d, 1H), 8.25 (dd, 1H), 7.59-7.57 (m, 2H), 7.07 (d, 1H), 6.99-6.96 (m, 2H), 3.91- 3.86 (m, 4H), 3.18-3.14 (m, 4H), 2.37 (d, 2H), 2.25-2.21 (m, 1H), 1.066 (t, 6H). LC-MS [M + H]⁺ 457.2322 |
| 2 | 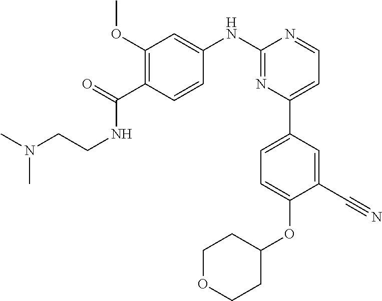 [SMILES: COc1cc(Nc2nccc(-c3ccc(OC4CCOCC4)c(C#N)c3)n2)ccc1C(=O)NCCN(C)C] | 4-({4-[3-cyano-4- (tetrahydero-2H- pyran-4- yloxy)phenyl]pyrimidin- 2-yl}amino)-N- [2- (dimethylamino) ethyl]-2- methoxybenzamide | ¹H NMR (DMSO-d₆) δ 10.1 (s, 1H), 9.30 (br s, 1H), 8.65-8.61 (m, 2H), 8.47 (dd, 1H), 8.41 (t, 1H), 8.00 (s, 1H), 7.88 (d, 1H), 7.60-7.56 (m, 2H), 7.38 (d, 1H), 4.96 (m, 1H), 4.00 (s, 3H), 3.90-3.85 (m, 2H), 3.67-3.62 (m, 2H), 3.60-3.54 (m, 2H), 3.29-3.24 (m, 2H), 2.85 (s, 6H), 2.08-2.01 (m, 2H), 1.73-1.66 (m, 2H); LC-MS [M + H]⁺ 517.2548 |
| 3 | 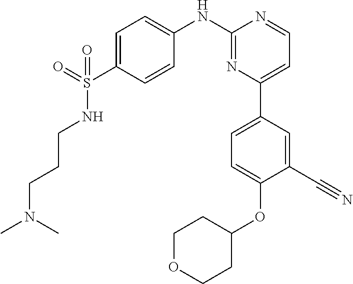 [SMILES: CN(C)CCCNS(=O)(=O)c1ccc(Nc2nccc(-c3ccc(OC4CCOCC4)c(C#N)c3)n2)cc1] | 4-({4-[3-cyano-4- (tetrahydro-2H- pyran-4- yloxy)phenyl]pyrimidin- 2-yl}amino)-N- [3- (dimethylamino) propyl] benzenesulfonamide | ¹H NMR (DMSO-d₆) δ 10.2 (s, 1H), 9.30 (br s, 1H), 8.64 (d, 1H), 8.58 (d, 1H), 8.48 (dd, 1H), 8.03 (d, 2H), 7.74 (d, 2H), 7.62-7.56 (m, 3H), 4.94-4.91 (m, 1H), 3.91-3.86 (m, 2H), 3.59-3.54 (m, 2H), 3.06- 3.02 (m, 2H), 2.80-2.75 (m, 2H), 2.74 (s, 6H), 2.09-2.02 (m, 2H), 1.79-1.66 (m, 4H); LC-MS [M + H]⁺ 537.2271 |
| 4 | 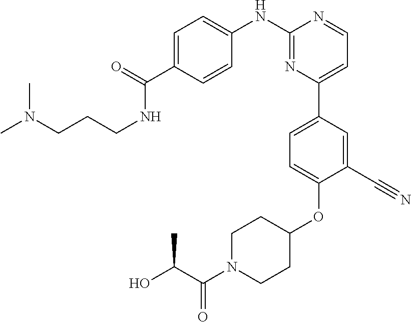 [SMILES: C[C@H](O)C(=O)N1CCC(Oc2ccc(-c3ccnc(Nc4ccc(C(=O)NCCCN(C)C)cc4)n3)cc2C#N)CC1] | 4-({4-[3-cyano-4- ({1-[(2S)-2- hydroxypropanoyl] piperidin-4- yl}oxy)phenyl]pyrimidin- 2-yl}amino)-N- [3- (dimethylamino) propyl]benzamide | ¹H NMR (DMSO-d₆) δ 10.2 (s, 1H), 9.44 (br s, 1H), 8.62 (d, 1H), 8.57 (d, 1H), 8.53-8.49 (m, 1H), 7.93- 7.83 (m, 4H), 7.59-7.56 (m, 2H), 5.06-4.98 (m, 2H), 4.50-4.44 (m, 1H), 3.86-3.66 (m, 2H), 3.58-3.48 (m, 2H), 3.36-3.30 (m, 2H), 3.14- 3.04 (m, 1H), 2.80 (s, 3H), 2.79 (s, 3H), 2.14-1.95 (m, 2H), 1.92-1.85 (m, 2H), 1.80-1.58 (m, 2H), 1.21 (d, 3H); LC-MS [M + H]⁺ 572.2979 |
| 5 | 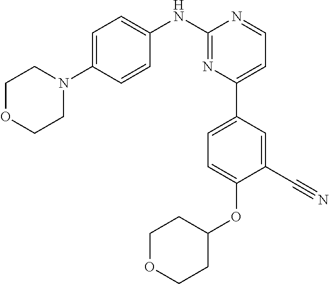 [SMILES: N#Cc1cc(-c2ccnc(Nc3ccc(N4CCOCC4)cc3)n2)ccc1OC1CCOCC1] | 5-(2-{[4-(Morpholin- 4- yl)phenyl]amino} pyrimidin-4-yl)-2- (tetrahydro-2H- pyran-4- yloxy)benzaonitrile | ¹H NMR (CDCl₃) δ 8.44 (d, 1H), 8.31 (d, 1H), 8.23-8.20 (m, 1H), 7.56-7.53 (m, 2H), 7.16 (br s, 1H), 7.07 (d, 1H), 7.02 (d, 1H), 6.97- 6.94 (m, 2H), 4.78-4.72 (m, 1H), 4.07-4.01 (m, 2H), 3.90-3.87 (m, 4H), 3.69-3.63 (m, 2H), 3.16-3.13 (m, 4H), 2.11-2.05 (m, 2H), 1.97- 1.88 (m, 2H). LC-MS [M + H]⁺ 458.2203 |
| 6 | 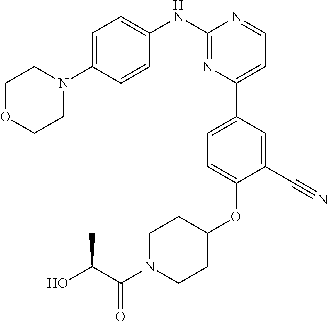 [SMILES: C[C@H](O)C(=O)N1CCC(Oc2ccc(-c3ccnc(Nc4ccc(N5CCOCC5)cc4)n3)cc2C#N)CC1] | 2-({1-[(2S)-2- Hydroxypropanoyl] piperidin-4-yl}oxy)-5- (2-{[4-(morpholin-4- yl)phenyl]amino} pyrimidin-4- yl)benzonitrile | ¹H NMR (MeOH-d₄) δ 8.50 (s, 1H), 8.45-8.42 (m, 2H), 7.82 (apparent d, 2H), 7.42 (apparent d, 4H), 5.03- 4.93 (m, 1H), 4.64-4.59 (m, 1H), 4.04-3.97 (m, 4H), 3.90-3.47 (m, 8H), 2.15-1.87 (m, 4H), 1.34 (d, 3H). LC-MS [M + H]⁺ 529.2426 |
| 7 | 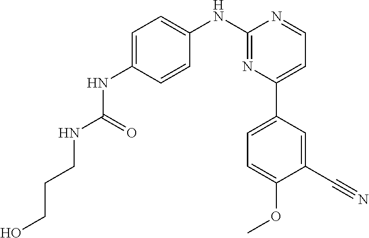 [SMILES: COc1ccc(-c2ccnc(Nc3ccc(NC(=O)NCCCO)cc3)n2)cc1C#N] | 1-(4-{[4-(3-Cyano-4- methoxyphenyl) pyrimidin-2- yl]amino}phenyl)-3- (3- hydroxypropyl)urea | ¹H NMR (DMSO-d₆) δ 9.55 (s, 1H), 8.53 (d, 1H), 8.52-8.46 (m, 2H), 8.36 (br s, 1H), 7.65-7.57 (m, 2H), 7.45 (d, 1H), 7.42 (d, 1H), 7.37- 7.30 (m, 2H), 6.08 (br s, 1H), 4.01 (s, 3H), 3.46 (t, 2H), 3.14 (t, 2H), 1.58 (quint, 2H); LC-MS [M + H]⁺ 419.1829 |
| 8 | 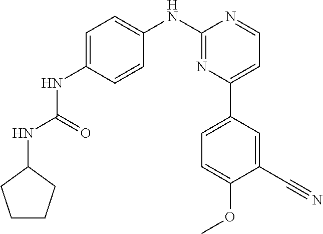 [SMILES: COc1ccc(-c2ccnc(Nc3ccc(NC(=O)NC4CCCC4)cc3)n2)cc1C#N] | 1-(4-{[4-(3-Cyano-4- methoxyphenyl) pyrimidin-2- yl]amino}phenyl)-3- cyclopentylurea | ¹H NMR (DMSO-d₆) δ 9.51 (s, 1H), 8.52 (d, 1H), 8.51-8.46 (m, 2H), 8.14 (s, 1H), 7.65-7.58 (m, 2H), 7.45 (d, 1H), 7.41 (d, 1H), 7.35- 7.28 (m, 2H), 6.08 (d, 1H), 4.01 (s, 3H), 3.93 (sextet, 1H), 1.90-1.75 (m, 2H), 1.70-1.45 (m, 4H), 1.40- 1.28 (m, 2H); LC-MS [M + H]⁺ 429.2035 |
| 9 | 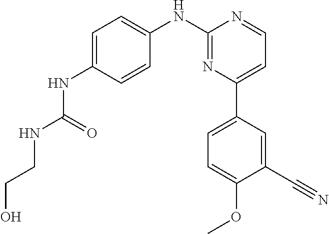 [SMILES: COc1ccc(-c2ccnc(Nc3ccc(NC(=O)NCCO)cc3)n2)cc1C#N] | 1-(4-{[4-(3-Cyano-4- methoxyphenyl) pyrimidin-2- yl]amino}phenyl)-3- (2-hydroxyethyl)urea | ¹H NMR (DMSO-d₆) δ 9.55 (s, 1H), 8.53 (d, 1H), 8.52-8.42 (m, 3H), 7.68-7.58 (m, 2H), 7.45 (d, 1H), 7.42 (d, 1H), 7.36-7.31 (m, 2H), 6.13 (br s, ,1H), 4.01 (s, 3H), 3.44 (t, 2H), 3.15 (t, 2H); LC-MS [M + H]⁺ 405.1669 |
| 10 | 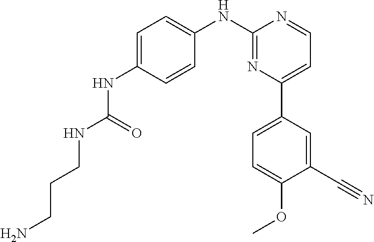 [SMILES: COc1ccc(-c2ccnc(Nc3ccc(NC(=O)NCCCN)cc3)n2)cc1C#N] | 1-(3-Aminopropyl)- 3-(4-{[4-(3-cyano-4- methoxyphenyl) pyrimidin-2- yl]amino}phenyl) urea | ¹H NMR (DMSO-d₆) δ 9.55 (s, 1H), 8.55-8.45 (m, 4H), 7.70 (br s, 3H), 7.62 (d, 2H), 7.48-7.40 (m, 2H), 7.34 (d, 2H), 6.32 (br s, 1H), 4.01 (s, 3H), 3.20-3.10 (m, 2H), 2.88- 2.76 (m, 2H), 1.71 (quint, 2H); LC- MS [M + H]⁺ 418.1990 |
| 11 | 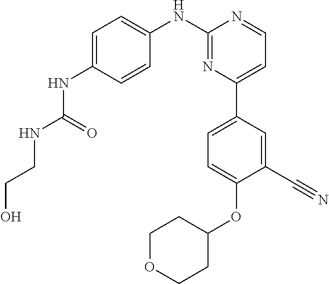 [SMILES: N#Cc1cc(-c2ccnc(Nc3ccc(NC(=O)NCCO)cc3)n2)ccc1OC1CCOCC1] | 1-[4-({4-[3-cyano-4- (tetrahydro-2H- pyran-4- yloxy)phenyl] pyrimidin-2- yl}amino)phenyl]-3- (2-hydroxyethyl)urea | ¹H NMR (DMSO-d₆) δ 9.54 (s, 1H), 8.56-8.54 (m, 1H), 8.54-8.46 (m, 1H), 8.45-8.42 (m, 2H), 7.65-7.60 (m, 2H), 7.55 (d, 1H), 7.42 (s, 1H), 7.36-7.30 (m, 2H), 6.14 (br s, 1H), 4.94 (sept, 1H), 3.94-3.84 (m, 2H), 3.55 (ddd, 2H), 3.44 (t, 2H), 3.19- 3.10 (m, 2H), 2.10-2.00 (m, 2H), 1.75-1.62 (m, 2H); LC-MS [M + H]⁺ 475.2079 |
| 12 | 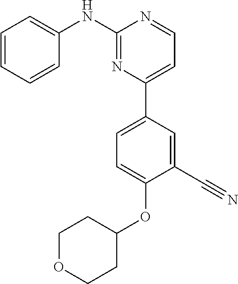 [SMILES: N#Cc1cc(-c2ccnc(Nc3ccccc3)n2)ccc1OC1CCOCC1] | 5-[2- (phenylamino)pyrimidin- 4-yl]-2- (tetrahydro-2H- pyran-4- yloxy)benzonitrile] | ¹H NMR (DMSO-d₆) δ 9.71 (s, 1H), 8.58-8.53 (m, 2H), 8.46 (dd, 1H), 7.83-7.78 (m, 2H), 7.56 (s, 1H), 7.48 (d, 1H), 7.35-7.28 (m, 2H), 7.01-6.95 (m, 1H), 4.95 (sept, 1H), 3.94-3.82 (m, 2H), 3.56 (ddd, 2H), 2.10-2.00 (m, 2H), 1.75-1.63 (m, 2H); LC-MS [M + H]⁺ 373.1592 |
| 13 | 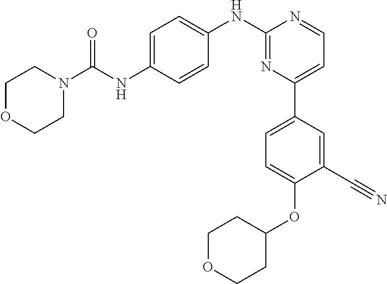 [SMILES: N#Cc1cc(-c2ccnc(Nc3ccc(NC(=O)N4CCOCC4)cc3)n2)ccc1OC1CCOCC1] | N-[4-({4-[3-cyano-4- (tetrahydro-2H- pyran-4- yloxy)phenyl] pyrimidin-2- yl}amino)phenyl] morpholin-4- carboxamide | ¹H NMR (DMSO-d₆) δ 9.58 (s, 1H), 8.53 (d, 1H), 8.51 (d, 1H), 8.48- 8.42 (m, 2H), 7.68-7.62 (m, 2H), 7.56 (d, 1H), 7.43 (d, 1H), 7.42- 4.36 (m, 2H), 4.94 (sept, 1H), 3.92- 3.83 (m, 2H), 3.58-3.65 (m, 4H), 3.55 (ddd, 2H), 3.45-3.38 (m, 4H), 2.10-1.98 (m, 2H), 1.62-1.76 (m, 2H); LC-MS [M + H]⁺ 501.2185 |
| 14 | 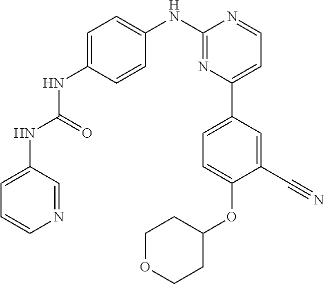 [SMILES: N#Cc1cc(-c2ccnc(Nc3ccc(NC(=O)Nc4cccnc4)cc3)n2)ccc1OC1CCOCC1] | 1-[4-({4-[3-cyano-4- (tetrahydro-2H- pyran-4- yloxy)phenyl] pyrimidin-2- yl}amino)phenyl]-3- pyridin-3-ylurea | ¹H NMR (DMSO-d₆) δ 9.68 (s, 1H), 9.65 (s, 1H), 9.22 (s, 1H), 9.04 (s, 1H), 8.57-8.52 (m, 2H), 8.48-8.42 (m, 2H), 8.30-8.25 (m, 1H), 7.82 (dd, 1H), 7.76-7.71 (m, 2H), 7.57 (d, 1H), 7.47-7.41 (m, 3H), 4.95 (sept, 1H), 3.92-3.84 (m, 2H), 3.56 (ddd, 2H), 2.10-2.00 (m, 2H), 1.76- 1.634 (m, 2H); LC-MS [M + H]⁺ 508.2116 |
| 15 | 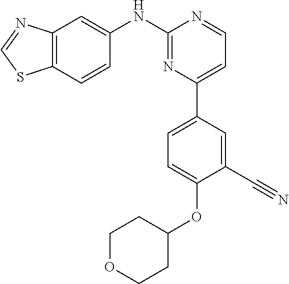 [SMILES: N#Cc1cc(-c2ccnc(Nc3ccc4scnc4c3)n2)ccc1OC1CCOCC1] | 5-[2-(1,3- benzothiazol-5- ylamino)pyrimidin- 4-yl]-2-(tetrahydro- 2H-pyran-4- yloxy)benzonitrile | ¹H NMR (DMSO-d₆) δ 9.99 (s, 1H), 9.37 (s, 1H), 8.76 (d, 1H), 8.62 (d, 1H), 8.58 (d, 1H), 8.49 (dd, 1H), 8.06 (d, 1H), 7.81 (dd, 1H), 7.59 (d, 1H), 7.54 (d, 1H), 4.97 (sept, 1H), 3.92-3.83 (m, 2H), 3.56 (ddd, 2H), 2.10-2.00 (m, 2H), 1.76-1.62 (m, 2H); LC-MS [M + H]⁺ 430.1328 |
| 16 | 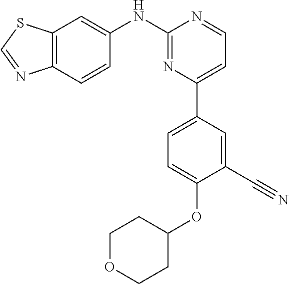 [SMILES: N#Cc1cc(-c2ccnc(Nc3ccc4ncsc4c3)n2)ccc1OC1CCOCC1] | 5-[2-(1,3- benzothiazol-6- ylamino)pyrimidin- 4-yl]-2-(tetrahydro- 2H-pyran-4- yloxy)benzonitrile | ¹H NMR (DMSO-d₆) δ 10.04 (s, 1H), 9.23 (s, 1H), 8.76 (d, 1H), 8.62 (d, 1H), 8.58 (d, 1H), 8.47 (dd, 1H), 8.02 (d, 1H), 7.81 (dd, 1H), 7.57 (dd, 1H), 7.54 (d, 1H), 4.96 (sept, 1H), 3.94-3.83 (m, 2H), 3.56 (ddd, 2H), 2.10-1.98 (m, 2H), 1.76- 1.63 (m, 2H); LC-MS [M + H]⁺ 430.1334 |
| 17 | 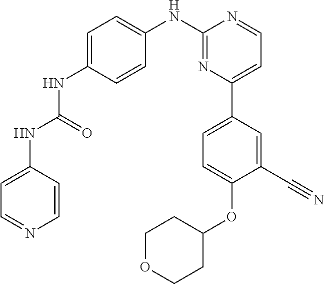 [SMILES: N#Cc1cc(-c2ccnc(Nc3ccc(NC(=O)Nc4ccncc4)cc3)n2)ccc1OC1CCOCC1] | 1-[4-({4-[3-cyano-4- (tetrahydro-2H- pyran-4- yloxy)phenyl] pyrimidin-2- yl}amino)phenyl]-3- pyridin-4-ylurea | ¹H NMR (DMSO-d₆) δ 11.04 (s, 1H), 9.92 (s, 1H), 9.70 (s, 1H), \8.61 (d, 2H), 8.57-8.52 (m, 2H), 8.46 (dd, 1H), 8.02-7.92 (m, 2H), 7.82-7.73 (m, 2H), 7.57 (d, 1H), 7.54-7.43 (m, 3H), 4.95 (sept, 1H), 3.94-3.82 (m, 2H), 3.62-3.50 (m, 2H), 1.97-2.04 (m, 2H), 1.78-1.60 (m, 2H); LC-MS [M + H]⁺ 508.2114 |
| 18 | 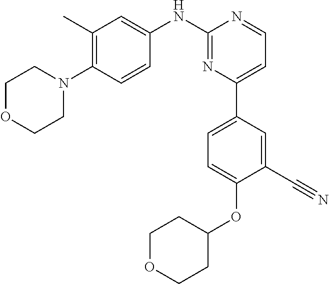 [SMILES: Cc1cc(Nc2nccc(-c3ccc(OC4CCOCC4)c(C#N)c3)n2)ccc1N1CCOCC1] | 5-(2-{[3-methyl-4- (morpholin-4- yl)phenyl]amino} pyrimidin-4-yl)-2- (tetrahydro-2H- pyran-4- yloxy)benzonitrile | ¹H NMR (DMSO-d₆) δ 9.57 (s, 1H), 8.55 (d, 1H), 5.20 (d, 1H), 8.44 (dd, 1H), 7.52-7.68 (m, 3H), 7.44 (d, 1H), 7.04 (d, 1H), 4.95 (sept, 1H), 3.92-3.83 (m, 2H), 3.79-3.71 (m, 4H), 3.56 (ddd, 2H), 2.84 (br s, 4H), 2.30 (s, 3H), 2.10-2.00 (m, 2H), 1.75-1.62 (m, 2H); LC-MS [M + H]⁺ 472.2332 |
| 19 | 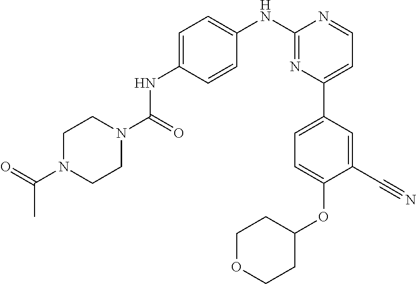 [SMILES: CC(=O)N1CCN(C(=O)Nc2ccc(Nc3nccc(-c4ccc(OC5CCOCC5)c(C#N)c4)n3)cc2)CC1] | 4-acetyl-N-[4-({4-[3- cyano-4-(tetrahydro- 2H-pyran-4- yloxy)phenyl] pyrimidin-2- yl}amino)phenyl] piperazine-1- carboxamide | ¹H NMR (DMSO-d₆) δ 9.59 (s, 1H), 8.56-8.50 (m, 3H), 8.44 (dd, 1H), 7.68-7.62 (m, 2H), 7.56 (d, 1H), 7.43 (d, 1H), 7.42-7.36 (m, 2H), 4.94 (sept, 1H), 3.92-3.83 (m, 2H), 3.55 (ddd, 2H), 3.47 (br s, 6H), 3.46-3.38 (m, 2H), 2.10-2.00 (m, 2H), 2.04 (s, 3H), 1.76-1.62 (m, 2H); LC-MS [M + H]⁺ 542.2510 |
| 20 | 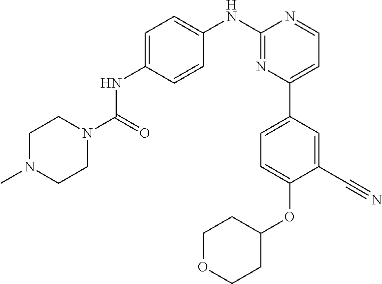 [SMILES: CN1CCN(C(=O)Nc2ccc(Nc3nccc(-c4ccc(OC5CCOCC5)c(C#N)c4)n3)cc2)CC1] | N-[4-({4-[3-cyano-4- (tetrahydro-2H- pyran-4- yloxy)phenyl] pyrimidin-2- yl}amino)phenyl]-4- methylpiperazine-1- carboxamide | ¹H NMR (DMSO-d₆) δ 9.84 (br s, 1H), 9.61 (s, 1H), 8.69 (s, 1H), 8.55-8.50 (m, 2H), 8.44 (dd, 1H), 7.67 (d, 2H), 7.55 (d, 1H), 7.44 (d, 1H), 7.38 (d, 1H), 4.95 (sept, 1H), 4.25 (d, 2H), 3.94-3.82 (m, 2H), 3.56 (ddd, 2H), 3.47 (d, 2H), 3.20- 2.95 (m, 5H), 2.84 (s, 3H), 2.10- 1.98 (m, 2H), 1.78-1.62 (m, 2H) LC-MS [M + H]⁺ 514.2549; |
| 21 | 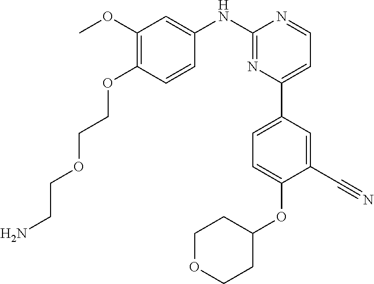 [SMILES: COc1cc(Nc2nccc(-c3ccc(OC4CCOCC4)c(C#N)c3)n2)ccc1OCCOCCN] | 5-[2-({4-[2-(2- aminoethoxy)ethoxy]- 3- methoxyphenyl} amino)pyrimidin-4-yl]-2- (tetrahydro-2H- pyran-4- yloxy)benzonitrile | ¹H NMR (DMSO-d₆) δ 9.58 (br s, 1H), 8.56 (d, 1H), 8.53 (d, 1H), 8.43 (dd, 1H), 7.81 (br s, 3H), 7.70 (br s, 1H, 7.54 (d, 1H), 7.44 (d, 1H), 7.20 (d, 1H), 6.94 (d, 1H), 4.95 (sept, 1H), 4.12-4.06 (m, 2H), 3.92-3.84 (m, 2H), 3.82 (s, 3H), 3.82-3.76 (m, 2H), 3.71-3.66 (m, 2H), 3.56 (ddd, 2H), 3.08-2.98 (m, 2H), 2.1-2.0 (m, 2H), 1.75-1.61 (m, 2H); LC-MS [M + H]⁺ 506.2394 |
| 22 | 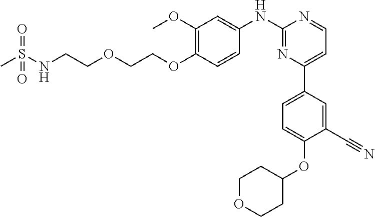 [SMILES: COc1cc(Nc2nccc(-c3ccc(OC4CCOCC4)c(C#N)c3)n2)ccc1OCCOCCNS(C)(=O)=O] | N-(2-{2-[4-({4-[3- cyano-4-(tetrahydro- 2H-pyran-4- yloxy)phenytl] pyrimidin-2-yl}amino]-2- methoxyphenoxy}ethyl) methansulfonamide | ¹H NMR (DMSO-d₆) δ 9.56 (s, 1H), 8.56 (d, 1H), 8.52 (d, 1H), 8.44 (dd, 1H), 7.65 (br s, 1H), 7.20 (d, 1H), 7.09 (t, 1H), 6.92 (d, 1H), 4.95 (sept, 1H), 4.07-4.03 (m, 2H), 3.91- 3.84 (m, 2H), 3.81 (s, 3H), 3.76- 3.72 (m, 2H), 3.60-3.52 (m, 4H), 3.14 (q, 2H), 2.93 (s, 3H), 2.10- 1.98 (m, 2H), 1.75-1.61 (m, 2H); LC-MS [M + H]⁺ 584.2170 |
| 23 | 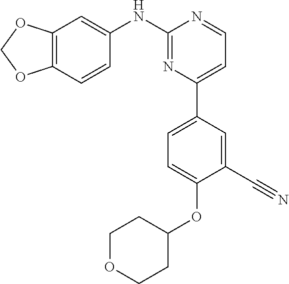 [SMILES: N#Cc1cc(-c2ccnc(Nc3ccc4c(c3)OCO4)n2)ccc1OC1CCOCC1] | 5-[2-(1,3- benzodioxol-5- ylamino)pyrimidin- 4-yl]-2-(tetrahydro- 2H-pyran-4- yloxy)benzonitrile | ¹H NMR (DMSO-d₆) δ 9.60 (s, 1H), 8.54-8.52 (m, 2H), 8.42 (dd, 1H), 7.56 (d, 1H), 7.53 (d, 1H), 7.44 (d, 1H), 7.16 (dd, 1H), 6.87 (d, 1H), 5.99 (s, 2H), 4.95 (sept, 1H), 3.93- 3.83 (m, 2H), 3.55 (ddd, 2H), 2.10- 1.98 (m, 2H), 1.74-1.62 (m, 2H); LC-MS [M + H]⁺ 417.1546 |
| 24 | 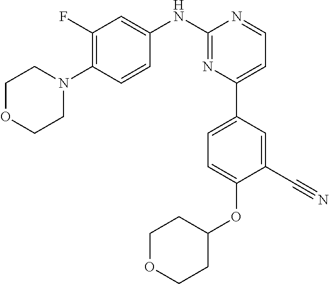 [SMILES: N#Cc1cc(-c2ccnc(Nc3ccc(N4CCOCC4)c(F)c3)n2)ccc1OC1CCOCC1] | 5-(2-{[3-fluoro-4- (morpholin-4- yl)phenyl]amino} pyrimidin-4-yl)-2- (tetrahydro-2H- pyran-4- yloxy)benzonitrile | ¹H NMR (DMSO-d₆) δ 9.77 (s, 1H), 8.55 (d, 1H), 8.54 (d, 1H), 8.44 (dd, 1H), 7.78 (dd, 1H), 7.56 (d, 1H), 7.56-7.44 (m, 2H), 7.02 (dd, 1H), 4.95 (sept, 1H), 3.92-3.82 (m, 2H), 3.78-3.70 (m, 4H), 3.56 (ddd, 2H), 3.00-2.92 (m, 4H), 2.10-2.00 (m, 2H), 1.75-1.63 (m, 2H); LC- MS [M + H]⁺ 476.2079 |
| 25 | 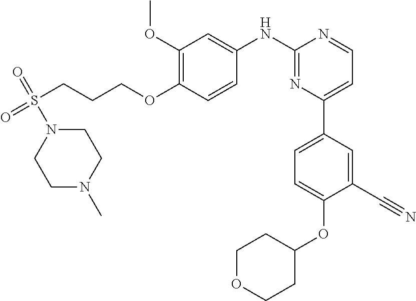 [SMILES: COc1cc(Nc2nccc(-c3ccc(OC4CCOCC4)c(C#N)c3)n2)ccc1OCCCS(=O)(=O)N1CCN(C)CC1] | 5-{2-[(3-methoxy-4- {3-[(4- methylpiperazin-1- yl)sulfonyl]propoxy} phenyl)amino] pyrimidin-4-yl}-2- (tetrahydro-2H- pyran-4- yloxy)benzonitrile | ¹H NMR (DMSO-d₆) δ 9.96 (br s, 1H), 9.60 (s, 1H), 8.56 (d, 1H), 8.53 (d, 1H), 8.44 (dd, 1H), 7.70 (br s, 1H), 7.55 (d, 1H), 7.44 (d, 1H), 7.21 (d, 1H), 6.94 (d, 1H), 4.95 (sept, 1H), 4.04 (t, 2H), 3.92- 3.75 (m, 4H), 3.83 (s, 3H), 3.60- 3.47 (m, 4H), 3.39-3.31 (m, 2H), 3.22-3.04 (m, 4H), 2.85 (s, 3H), 2.15-1.98 (m, 4H), 1.75-1.62 (m, 2H); LC-MS [M + H]⁺ 623.2646 |
| 26 | 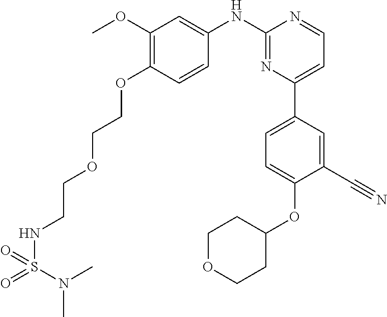 [SMILES: COc1cc(Nc2nccc(-c3ccc(OC4CCOCC4)c(C#N)c3)n2)ccc1OCCOCCNS(=O)(=O)N(C)C] | N′-(2-{2-[4-({4-[3- cyano-4-(tetrahydro- 2H-pyran-4- yloxy)phenyl] pyrimidin-2-yl} amino)-2- methoxyphenoxy] ethoxy}ethyl)-N,N- dimethylsulfuric diamide | ¹H NMR (DMSO-d₆) δ 9.56 (br s, 1H), 8.56 (d, 1H), 8.562 (d, 1H), 8.44 (dd, 1H), 7.65 (br s, 1H), 7.55 (d, 1H), 7.43 (d, 1H), 7.26 (t, 1H), 7.20 (dd, 1H), 7.93 (d, 1H), 4.95 (sept, 1H), 4.08-4.02 (m, 2H), 3.92- 3.83 (m, 2H), 3.81 (s, 3H), 3.75- 3.70 (m, 2H), 3.60-3.50 (m, 4H), 3.08 (q, 2H), 2.66 (s, 6H), 2.08- 2.10 (m, 2H), 1.75-1.63 (m, 2H); LC-MS [M + H]⁺ 613.2438 |
| 27 | 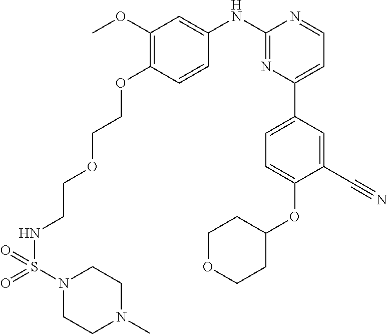 [SMILES: COc1cc(Nc2nccc(-c3ccc(OC4CCOCC4)c(C#N)c3)n2)ccc1OCCOCCNS(=O)(=O)N1CCN(C)CC1] | N-(2-{2-[4-({4-[3- cyano-4-(tetrahydro- 2H-pyran-4- yloxy)phenyl] pyrimidin-2-yl} amino)-2- methoxyphenoxy] ethoxy}ethyl)-4- methylpiperazine-1- sulfonamide | ¹H NMR (DMSO-d₆) δ 9.73 (br s, 1H), 9.57 (s, 1H), 8.56 (d, 1H), 8.53 (d, 1H), 8.43 (dd, 1H), 7.73 (t, 1H), 7.68 (br s, 1H), 7.54 (d, 1H), 7.44 (d, 1H), 7.20 (d, 1H), 6.93 (d, 1H), 4.95 (sept, 1H), 4.10-4.02 (m, 2H), 3.92-3.83 (m, 2H), 3.82 (s, 3H), 3.78-3.71 (m, 2H), 3.51 (br d, 2H), 3.60-3.51 (m, 4H), 3.48 (br d, 2H), 3.10-3.00 (m, 4H), 3.00-2.86 (m, 2H), 2.82 (br s, 3H), 2.10-1.98 (m, 2H), 1.74-1.63 (m, 2H); LC- MS [M + H]⁺ 668.2851 |
| 28 | 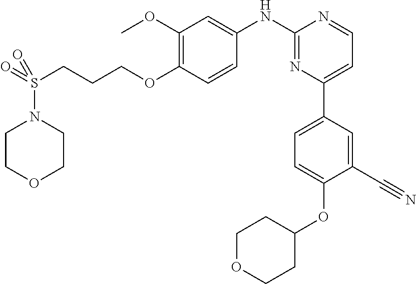 [SMILES: COc1cc(Nc2nccc(-c3ccc(OC4CCOCC4)c(C#N)c3)n2)ccc1OCCCS(=O)(=O)N1CCOCC1] | 5-[2-({3-methoxy-4- [3-(morpholin-4- ylsulfonyl)propoxy] phenyl}amino) pyrimidin-4-yl]-2- (tetrahydro-2H- pyran-4- yloxy)benzonitrile | ¹H NMR (DMSO-d₆) δ 9.59 (s, 1H), 8.56 (d, 1H), 8.53 (d, 1H), 8.44 (dd, 1H), 7.68 (br s, 1H), 7.55 (d, 1H), 7.44 (d, 1H), 7.21 (d, 1H), 6.94 (d, 1H), 4.95 (sept, 1H), 4.04 (t, 2H), 3.92-3.82 (m, 2H), 3.82 (s, 3H), 3.67-3.62 (m, 4H), 3.56 (ddd, 2H), 3.28-3.21 (m, 2H), 3.20-3.15 (m, 4H), 2.14-1.98 (m, 4H), 1.74- 1.62 (m, 2H); LC-MS [M + H]⁺ 610.2327 |
| 29 | 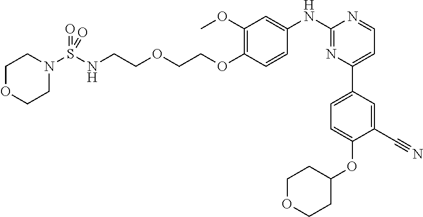 [SMILES: COc1cc(Nc2nccc(-c3ccc(OC4CCOCC4)c(C#N)c3)n2)ccc1OCCOCCNS(=O)(=O)N1CCOCC1] | N-(2-{2-[4-({4-[3- cyano-4-(tetrahydro- 2H-pyran-4- yloxy)phenyl] pyrimidin-2-yl}amino)-2- methoxyphenoxy] ethoxy}ethyl) morpholine- 4-sulfonamide | ¹H NMR (DMSO-d₆) δ 9.56 (s, 1H), 8.56 (d, 1H), 8.52 (dd, 1H), 8.44 (dd, 1H), 7.66 (br s, 1H), 7.54 (d, 1H), 7.48-7.40 (m, 2H), 7.20 (d, 1H), 6.92 (d, 1H), 4.95 (sept, 1H), 4.08-4.02 (m, 2H), 3.92-3.83 (m, 2H), 3.81 (s, 3H), 3.75-3.70 (m, 2H), 3.63-3.57 (m, 5H), 3.57-3.50 (m, 3H), 3.10 (q, 2H), 3.04-2.97 (m, 4H), 2.10-1.98 (m, 2H), 1.62- 1.74 (m, 2H); C-MS LC-MS [M + H]⁺ 655.2525 |
| 30 | 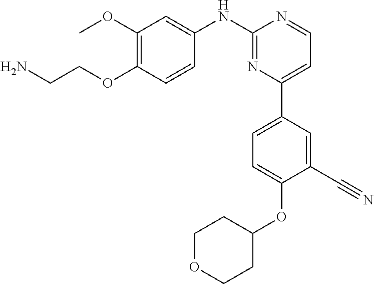 [SMILES: COc1cc(Nc2nccc(-c3ccc(OC4CCOCC4)c(C#N)c3)n2)ccc1OCCN] | 5-(2-{[4-(2- aminoethoxy)-3- methoxyphenyl]amino} pyrimidin-4-yl)-2- (tetrahydro-2H- pyran-4- yloxy)benzonitrile | ¹H NMR (DMSO-d₆) δ 9.64 (s, 1H), 8.57 (d, 1H), 8.54 (d, 1H), 8.44 (dd, 1H), 7.96 (br s, 3H), 7.76 (s, 1H), 7.54 (d, 1H), 7.46 (d, 1H), 7.22 (d, 1H), 7.01 (d, 1H), 4.96 (sept, 1H), 4.10 (t, 2H), 3.92-3.80 (m, 2H), 3.85 (s, 3H), 3.56 (ddd, 2H), 3.22-3.12 (m, 2H), 2.10-1.98 (m, 2H), 1.75-1.62 (m, 2H); LC-MS [M + H]⁺ 462.2132 |
| 31 | 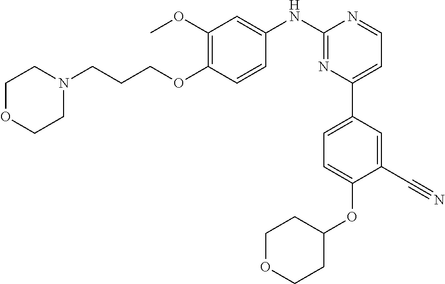 [SMILES: COc1cc(Nc2nccc(-c3ccc(OC4CCOCC4)c(C#N)c3)n2)ccc1OCCCN1CCOCC1] | 5-[2-({3-methoxy-4- [3-(morpholin-4- tl)propoxy]phenyl} amino)pyrimidin-4- yl]-2-(tetrahydro-2H- pyran-4- yloxy)benzonitrile | ¹H NMR (DMSO-d₆) δ 9.60 (s, 1H), 8.56 (d, 1H), 8.53 (d, 1H), 8.43 (dd, 1H), 7.71 (br s, 1H), 7.54 (d, 1H), 7.44 (d, 1H), 7.21 (d, 1H), 6.95 (d, 1H), 4.95 (sept, 1H), 4.08- 3.95 (m, 4H), 3.92-3.78 (m, 2H), 3.83 (s, 3H), 3.65 (t, 2H), 3.60-3.48 (m, 4H), 3.36-3.25 (m, 2H), 3.18- 3.05 (m, 2H), 2.18-1.99 (m, 4H), 1.75-1.62 (m, 2H); LC-MS [M + H]⁺ 546.2714 |
| 32 | 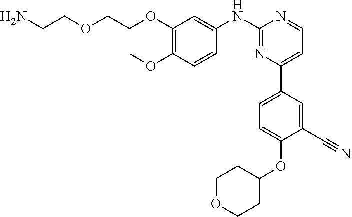 [SMILES: COc1ccc(Nc2nccc(-c3ccc(OC4CCOCC4)c(C#N)c3)n2)cc1OCCOCCN] | 5-[2-({3-[2-(2- aminoethoxy)ethoxy]- 4- methoxyphenyl} amino) pyrimidin-4-yl]-2- (tetrahydro-2H- pyran-4- yloxy)benzonitrile | ¹H NMR (DMSO-d₆) δ 9.54 (s, 1H), 8.54 (d, 1H), 8.52 (d, 1H), 8.43 (dd, 1H), 7.78 (br s, 3H), 7.60 (br s, 1H), 7.54 (d, 1H), 7.43 (d, 1H), 7.27 (dd, 1H), 6.94 (d, 1H), 4.95 (sept, 1H), 4.18-4.10 (m, 2H), 3.90- 3.80 (m, 4H), 3.75 (s, 3H), 3.72- 3.68 (m, 2H), 3.56 (ddd, 2H), 3.08- 2.98 (m, 2H), 2.10-1.98 (m, 2H), 1.74-1.62 (m, 2H); LC-MS [M + H]⁺ 506.2402 |
| 33 | 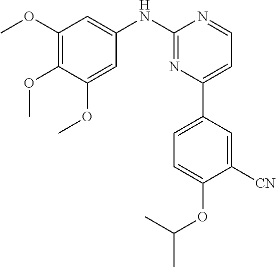 [SMILES: COc1cc(Nc2nccc(-c3ccc(OC(C)C)c(C#N)c3)n2)cc(OC)c1OC] | 2-(Propan-2-yloxy)- 5-{2-[(3,4,5- trimethoxyphenyl) amino]pyrimidin-4- yl}benzonitrile | ¹H NMR (CDCl₃) δ 8.45 (d, 1H), 8.38 (d, 1H), 8.22-8.19 (m, 1H), 7.42 (s, 1H), 7.08-7.02 (m, 4H), 4.77-4.74 (m, 1H), 3.93 (s, 6H), 3.85 (s, 3H), 1.45 (d, 6H). LC-MS [M + H]⁺ 421.2320 |
| 34 | 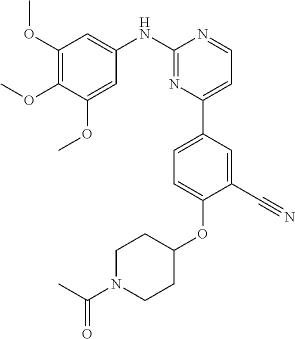 [SMILES: COc1cc(Nc2nccc(-c3ccc(OC4CCN(C(C)=O)CC4)c(C#N)c3)n2)cc(OC)c1OC] | 2-[(1- acetylpiperidin-4- yl)oxy]-5-{2-[(3,4,5- trimethoxyphenyl) amino]pyrimidin-4- yl}benzonitrile | ¹H NMR (DMSO-d₆) δ 9.61 (s, 1H), 8.59-8.55 (m, 2H), 8.47-8.44 (m, 1H), 7.56 (d, 1H), 7.47 (d, 1H), 7.28 (s, 2H), 5.20-4.85 (m, 1H), 3.81 (s, 6H), 3.76-3.69 (m, 2H), 3.63 (s, 3H), 3.47-3.41 (m, 2H), 2.04 (s, 3H), 2.00-1.90 (m, 2H), 1.80-1.72 (m, 1H), 1.68-1.58 (m, 1H). LC-MS [M + H]⁺ 504.2133 |
| 35 | 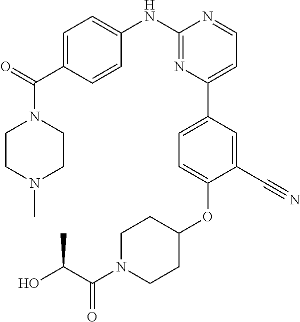 [SMILES: C[C@H](O)C(=O)N1CCC(Oc2ccc(-c3ccnc(Nc4ccc(C(=O)N5CCN(C)CC5)cc4)n3)cc2C#N)CC1] | 2-({1-[(2S)-2- hydroxypropanoyl] piperidin-4-yl}oxy)-5- [2-({4-[(4- methylpierazin-1- yl)carbonyl]phenyl} amino)pyrimidin-4- yl]benzonitrile | ¹H NMR (DMSO-d₆) δ 10.2 (s, 1H), 9.44 (br s, 1H), 8.62 (d, 1H), 8.57 (d, 1H), 8.53-8.49 (m, 2H), 7.93- 7.83 (m, 4H), 7.59-7.56 (m, 2H), 5.06-4.98 (m, 2H), 4.50-4.44 (m, 1H), 3.86-3.66 (m, 2H), 3.58-3.48 (m, 2H), 3.36-3.30 (m, 2H), 3.14- 3.04 (m, 1H), 2.80 (s, 3H), 2.79 (s, 3H), 2.14-1.95 (m, 2H), 1.92-1.85 (m, 2H), 1.80-1.58 (m, 2H), 1.21 (d, 3H). LC-MS [M + H]⁺ 570.2814 |
| 36 | 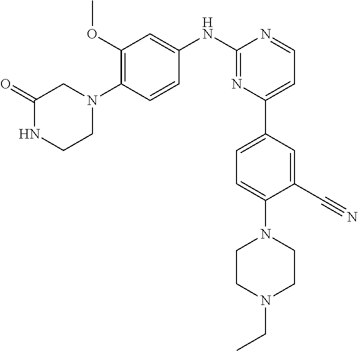 [SMILES: CCN1CCN(c2ccc(-c3ccnc(Nc4ccc(N5CCNC(=O)C5)c(OC)c4)n3)cc2C#N)CC1] | 2-(4-Ethylpiperazin- 1-yl)-5-(2-{[3- methoxy-4-(3- oxopiperazin-1- yl)phenyl]amino} pyrimidin-4- yl)benzonitrile | LC-MS [M + H]⁺ 513.2563 |
| 37 | 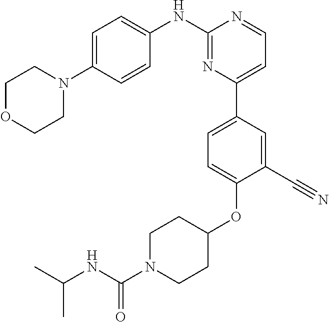 [SMILES: CC(C)NC(=O)N1CCC(Oc2ccc(-c3ccnc(Nc4ccc(N5CCOCC5)cc4)n3)cc2C#N)CC1] | 4-[2-Cyano-4-(2-{[4- (morpholin-4- yl)phenyl]amino} pyrimidin-4- yl)phenoxy]-N- (propan-2- yl)piperidine-1- carboxamide | ¹H NMR (DMSO-d₆) δ 9.45 (s, 1H), 8.51 (d, 1H), 8.49 (d, 1H), 8.45- 8.42 (m, 1H), 7.65-7.62 (m, 2H), 7.53 (d, 1H), 7.39 (d, 1H), 6.94- 6.91 (m, 2H), 6.25 (d, 1H), 4.95- 4.89 (m, 1H), 3.79-3.73 (m, 5H), 3.64-3.57 (m, 2H), 3.27-3.21 (m, 2H), 3.06-3.03 (m, 4H), 1.97-1.91 (m, 2H), 1.64-1.58 (m, 2H), 1.06 (d, 6H). LC-MS [M + H]⁺ 542.2765 |
| 38 | 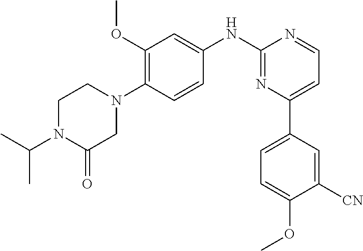 [SMILES: COc1ccc(-c2ccnc(Nc3ccc(N4CCN(C(C)C)C(=O)C4)c(OC)c3)n2)cc1C#N] | 2-Methoxy-5-[2-({3- methoxy-4-[3-oxo-4- (propan-2- yl)piperazin-1- yl]phenyl}amino) pyrimidin-4- yl]benzonitrile | ¹H NMR (CDCl₃) δ 8.46 (d, 1H), 8.36 (d, 1H), 8.27-8.24 (m, 1H), 7.56 (bd, 1H), 7.24 (s, 1H), 7.09- 7.03 (m, 3H), 6.89 (d, 1H), 4.96- 4.92 (m, 1H), 4.02 (s, 3H), 3.96 (s, 3H), 3.80 (s, 2H), 3.37-3.32 (m, 4H), 1.17 d, 6H). LC-MS [M + H]⁺ 473.2312 |
| 39 | 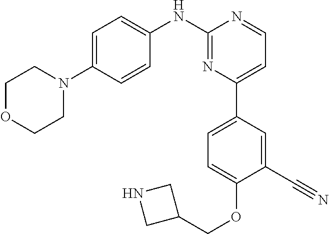 [SMILES: N#Cc1cc(-c2ccnc(Nc3ccc(N4CCOCC4)cc3)n2)ccc1OCC1CNC1] | 2-(azetidin-3- ylmethoxy)-5-(2-{[4- (morpholin-4- yl)phenyl]amino} pyrimidin-4- yl)benzonitrile | ¹H NMR (DMSO-d₆) δ 9.48 (s, 1H), 8.51-8.44 (m, 3H), 7.64 (d, 2H), 7.47 (d, 1H), 7.39 (d, 1H), 6.92 (d, 2H), 4.34 (d, 2H), 3.76-3.67 (m, 6H), 3.42-3.36 (m, 2H), 3.06-3.03 (m, 4H), 2.78-2.73 (m, 1H). LC- MS [M + H]⁺ 443.2141 |
| 40 | 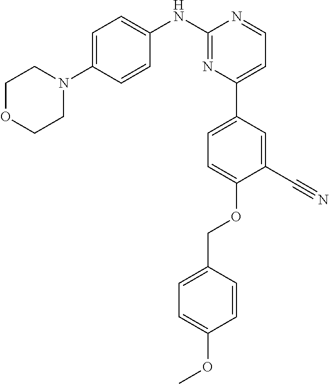 [SMILES: COc1ccc(COc2ccc(-c3ccnc(Nc4ccc(N5CCOCC5)cc4)n3)cc2C#N)cc1] | 2-[(4- Methoxybenzyl)oxy]- 5-(2-{[4- (morpholin-4- yl)phenyl]amino} pyrimidin-4- yl)benzonitrile | ¹H NMR (CDCl₃) δ 8.43 (d, 1H), 8.31 (d, 1H), 8.21-8.17 (m, 1H), 7.56-7.52 (m, 2H), 7.42-7.38 (m, 2H), 7.11 (d, 1H), 7.04 (br s, 1H), 7.00 (d, 1H), 6.97-6.92 (m, 3H), 5.23 (s, 3H), 3.90-3.87 (m, 4H), 3.16-3.13 (m, 4H). LC-MS [M + H]⁺ 494.2586 |
| 41 | 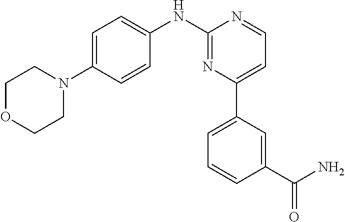 [SMILES: NC(=O)c1cccc(-c2ccnc(Nc3ccc(N4CCOCC4)cc3)n2)c1] | 3-(2-{[4-(Morpholin- 4- yl)phenyl]amino} pyrimidin-4- yl)benzamide | ¹H NMR (DMSO-d₆) δ 9.49 (s, 1H), 8.62-8.61 (m, 1H), 8.53 (d, 1H), 8.29 (d, 1H), 8.14 (br s, 1H), 8.02 (d, 1H), 7.70-7.61 (m, 4H), 7.50 (br s, 1H), 7.40 (d, 1H), 6.94-6.91 (m, 2H), 3.76-3.73 (m, 4H), 3.06-3.03 (m, 4H). LC-MS [M + H]⁺ 376.1803 |
| 42 | 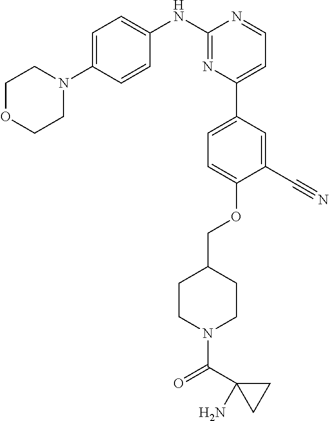 [SMILES: N#Cc1cc(-c2ccnc(Nc3ccc(N4CCOCC4)cc3)n2)ccc1OCC1CCN(C(=O)C2(N)CC2)CC1] | 2-({1-[(1- aminocyclopropyl) carbonyl]piperidin-4- yl}methoxy)-5-(2- {[4-(morpholin-4- yl)phenyl]amino} pyrimidin-4- yl)benzonitrile | ¹H NMR (DMSO-d₆) δ 9.56 (s, 1H), 8.77 (br s, 3H), 8.53-8.45 (m, 3H), 7.68 (d, 2H), 7.47-7.41 (m, 2H), 7.02 (d, 2H), 4.27-4.21 (m, 2H), 4.13 (d, 2H), 3.79-3.76 (m, 4H), 3.13-3.10 (m, 4H), 3.20-2.95 (m, 2H), 2.24-2.14 (m, 1H), 1.91-1.85 (m, 2H), 1.39-1.16 (m, 6H). LC- MS [M + H]⁺ 554.2770 |
| 43 | 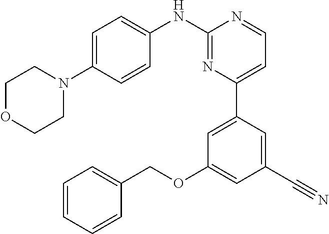 [SMILES: N#Cc1cc(OCc2ccccc2)cc(-c2ccnc(Nc3ccc(N4CCOCC4)cc3)n2)c1] | 3-(Benzyloxy)-5-(2- {[4-(morpholin-4- yl)phenyl]amino} pyrimidin-4- yl)benzonitrile | ¹H NMR (DMSO-d₆) δ 9.51 (s, 1H), 8.55 (d, 1H), 8.18-8.13 (m, 2H), 7.71-7.70 (m, 1H), 7.64-7.60 (m, 2H), 7.51-7.36 (m, 6H), 6.94-6.90 (m, 2H), 5.28 (s, 2H), 3.74-3.71 (m, 4H), 3.04-3.01 (m, 4H). LC-MS [M + H]⁺ 464.1978 |
| 44 | 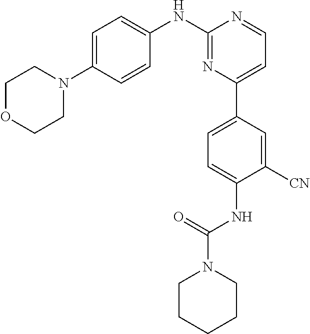 [SMILES: N#Cc1cc(-c2ccnc(Nc3ccc(N4CCOCC4)cc3)n2)ccc1NC(=O)N1CCCCC1] | N-[2-cyano-4-(2-{[4- (morpholin-4- yl)phenyl]amino} pyrimidin-4- yl)phenyl]piperidine- 1-carboxamide | ¹H NMR (CDCl₃) δ 8.43 (m, 2H), 8.28 (d, 1H), 8.205 (dd, 1H), 7.55 (d, 2H), 7.21 (m, 3H), 6.95- 7.04 (m, 3H), 3.88 (m, 4H), 3.54 (bs, 4H), 1.665 (m, 6H). LC-MS [M + H]⁺ 484.2432 |
| 45 | 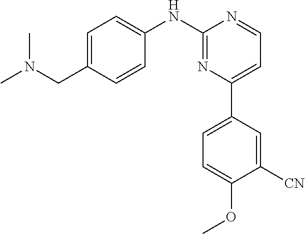 [SMILES: COc1ccc(-c2ccnc(Nc3ccc(CN(C)C)cc3)n2)cc1C#N] | 5-[2-({4- [(Dimethylamino) methyl]phenyl}amino) pyrimidin-4-yl]-2- methoxybenzonitrile | ¹H NMR (CDCl₃) δ 8.47 (d, 1H), 8.30-8.28 (m, 2H), 7.63 (d, 2H), 7.31 (d, 2H), 7.23 (s, 1H), 7.10- 7.06 (m, 2H), 4.02 (s, 3H), 3.42 (s, 2H), 2.26 (s, 6H). LC-MS [M + H]⁺ 360.3 |
| 46 | 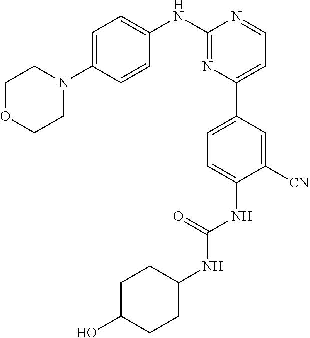 [SMILES: N#Cc1cc(-c2ccnc(Nc3ccc(N4CCOCC4)cc3)n2)ccc1NC(=O)NC1CCC(O)CC1] | 1-[2-cyano-4-(2-{[4- (morpholin-4- yl)phenyl]amino} pyrimidin-4-yl)phenyl]- 3-(4- hydroxycyclohexyl) urea | ¹H NMR (CDCl₃) δ 8.49 (d, 1H), 8.39 (d, 1H), 8.26 (d, 1H), 8.19 (dd, 1H), 7.57 (dd, 2H), 7.05 (d, 1H), 6.97 (dd, 2H), 3.89 (m, 4H), 3.62 (m, 2H), 3.15 (m, 4H), 2.04 (m, 4H), 1.35 (m, 4H). LC-MS [M + H]⁺ 514.2544 |
| 47 | 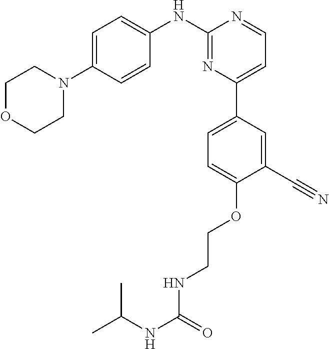 [SMILES: CC(C)NC(=O)NCCOc1ccc(-c2ccnc(Nc3ccc(N4CCOCC4)cc3)n2)cc1C#N] | 1-{2-[2-cyano-4-(2- {[4-(morpholin-4- yl)phenyl]amino} pyrimidin-4- yl)phenoxy]ethyl}-3- propan-2-ylurea | ¹H NMR (DMSO-d₆) δ 9.46 (s, 1H), 8.52-8.43 (m, 3H), 7.63 (d, 2H), 7.47 (d, 1H), 7.39 (d, 1H), 6.92 (d, 2H), 5.98-5.93 (m, 2H), 4.22 (t, 2H), 3.75-3.72 (m, 4H), 3.71-3.64 (m, 1H), 3.45-3.40 (m, 2H), 3.06- 3.03 (m, 4H), 1.02 (d, 6H). LC-MS [M + H]⁺ 502.2418 |
| 48 | 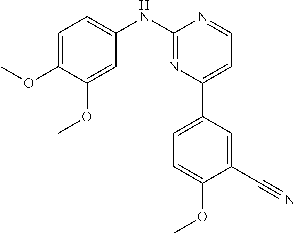 [SMILES: COc1ccc(-c2ccnc(Nc3ccc(OC)c(OC)c3)n2)cc1C#N] | 5-{2-[(3,4- Dimethoxyphenyl) amino]pyrimidin-4-yl}- 2- methoxybenzonitrile | ¹H NMR (CDCl₃) δ 8.45 (d, 1H), 8.34 (d, 1H), 8.27-8.24 (m, 1H), 7.47 (d, 1H), 7.13 (br s, 1H), 7.09- 7.02 (m, 3H), 6.88 (d, 1H), 4.02 (s, 3H), 3.95 (s, 3H), 3.90 (s, 3H). LC-MS [M + H]⁺ 363.1477 |
| 49 | 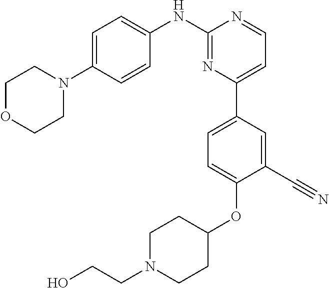 [SMILES: N#Cc1cc(-c2ccnc(Nc3ccc(N4CCOCC4)cc3)n2)ccc1OC1CCN(CCO)CC1] | 2-{[1-(2- Hydroxyethyl) piperidin-4-yl]oxy}- 5-(2- {[4-(morpholin-4- yl)phenyl]amino} pyrimidin-4- yl)benzonitrile | ¹H NMR (CDCl₃) δ 8.44 (d, 1H), 8.31 (d, 1H), 8.26-8.23 (m, 1H), 7.56-7.52 (m, 2H), 7.12 (br s, 1H), 7.10 (d, 1H), 7.02 (d, 1H), 6.97- 6.95 (m, 2H), 4.81 (br s, 1H), 4.20 (br s, 1H), 3.90-3.85 (m, 4H), 3.16- 3.13 (m, 4H), 3.12-3.05 (m, 4H), 2.93 (t, 2H), 2.50-2.40 (m, 2H), 2.15-2.09 (m, 2H). LC-MS [M + H]⁺ 501.2535 |
| 50 | 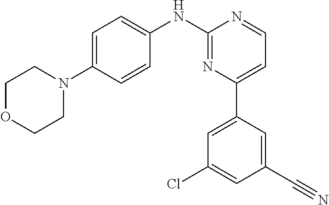 [SMILES: N#Cc1cc(Cl)cc(-c2ccnc(Nc3ccc(N4CCOCC4)cc3)n2)c1] | 3-Chloro-5-(2-{[4- (morpholin-4- yl)phenyl]amino} pyrimidin-4- yl)benzonitrile | ¹H NMR (DMSO-d₆) δ 9.58 (s, 1H), 8.59-8.52 (m, 3H), 8.24-8.23 (m, 1H), 7.63-7.60 (m, 2H), 7.51 (d, 1H), 6.94-6.92 (m, 2H), 3.76-3.73 (m, 4H), 3.06-3.04 (m, 4H). LC- MS [M + H]⁺ 392.1311 |
| 51 | 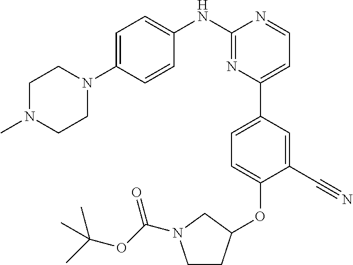 [SMILES: CN1CCN(c2ccc(Nc3nccc(-c4ccc(OC5CCN(C(=O)OC(C)(C)C)C5)c(C#N)c4)n3)cc2)CC1] | tert-butyl 3-[2- cyano-4-(2-{[4-(4- methylpiperazin-1- yl)phenyl]amino} pyrimidin-4- yl)phenoxy] pyrrolidine-1- carboxylate | LC-MS [M + H]⁺ 556.30 |
| 52 | 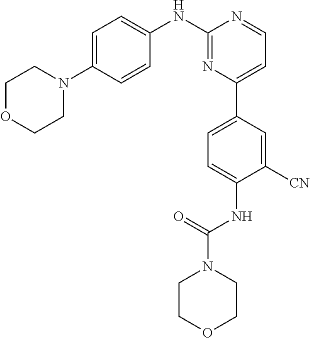 [SMILES: N#Cc1cc(-c2ccnc(Nc3ccc(N4CCOCC4)cc3)n2)ccc1NC(=O)N1CCOCC1] | N-[2-cyano-4-(2-{[4- (morpholin-4- yl)phenyl]amino} pyrimidin-4- yl)phenyl]morpholin- 4-carboxamide | ¹H NMR (CDCl₃) δ 8.40 (m, 2H), 8.30 (d, 1H), 8.22 (dd, 1H), 7.55 (dd, 2H), 7.16 (s, 1H), 7.095 (s, 1H), 7.04 (d, 1H), 6.96 (dd, 2H), 3.88 (m, 4H), 3.80 (m, 4H), 3.57 (m, 4H), 3.15 (m, 4H). LC-MS [M + H]⁺ 486.2223 |
| 53 | 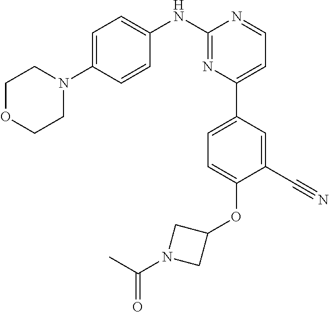 [SMILES: CC(=O)N1CC(Oc2ccc(-c3ccnc(Nc4ccc(N5CCOCC5)cc4)n3)cc2C#N)C1] | 2-[(1-acetylazetidin- 3-yl)oxy]-5-(2-{[4- (morpholin-4- yl)phenyl]amino} pyrimidin-4- yl)benzonitrile | ¹H NMR (DMSO-d₆) δ 9.48 (s, 1H), 8.56 (d, 1H), 8.50 (d, 1H), 8.46- 8.42 (m, 1H), 7.65-7.62 (m, 2H), 7.40 (d, 1H), 7.19 (d, 1H), 6.92 (d, 2H), 5.29-5.25 (m, 1H), 4.65-4.61 (m, 1H), 4.40-4.35 (m, 1H), 4.24- 4.20 (m, 1H), 3.87-3.84 (m, 1H), 3.76-3.73 (m, 4H), 3.06-3.03 (m, 4H), 1.82 (s, 3H). LC-MS [M + H]⁺ 471.2116 |
| 54 | 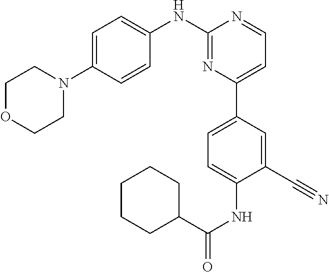 [SMILES: N#Cc1cc(-c2ccnc(Nc3ccc(N4CCOCC4)cc3)n2)ccc1NC(=O)C1CCCCC1] | N-[2-cyano-4-(2-{[4- (morpholin-4- yl)phenyl]amino} pyrimidin-4- yl)phenyl] cyclohexane- carboxamide | ¹H NMR (CDCl₃) δ 8.63 (d, 1H), 8.45 (d, 1H), 8.33 (d, 1H), 8.25- 8.22 (m, 1H), 7.80 (br s, 1H), 7.56- 7.53 (m, 2H), 7.14 (br s, 1H), 7.05 (d, 1H), 6.98-6.95 (m, 2H), 3.90- 3.88 (m, 4H), 3.17-3.14 (m, 4H), 2.41-2.33 (m, 1H), 2.06-2.00 (m, 2H), 1.90-1.85 (m, 2H), 1.76-1.22 (m, 6H). LC-MS [M + H]⁺ 483.2554 |
| 55 | 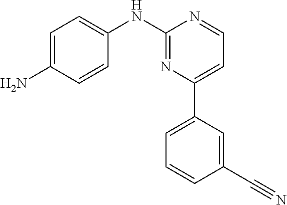 [SMILES: N#Cc1cccc(-c2ccnc(Nc3ccc(N)cc3)n2)c1] | 3-{2-[(4- Aminophenyl)amino] pyrimidin-4- yl}benzonitrile | ¹H NMR (CDCl₃) δ 8.47 (d, 1H), 8.36-8.35 (m, 1H), 8.27-8.24 (m, 1H), 7.78-7.75 (m, 1H), 7.62-7.58 (m, 1H), 7.41-7.38 (m, 2H), 7.07 (d, 1H), 7.02 (br s, 1H), 6.75-6.73 (m, 2H), 3.62 (br s, 2H). LC-MS [M + H]⁺ 288.1251 |
| 56 | 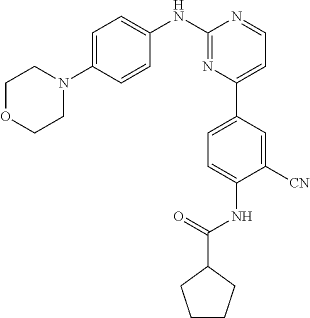 [SMILES: N#Cc1cc(-c2ccnc(Nc3ccc(N4CCOCC4)cc3)n2)ccc1NC(=O)C1CCCC1] | N-[2-cyano-4-(2-{[4- (morpholin-4- yl)phenyl]amino} pyrimidin-4- yl)phenyl] cyclopentane- carboxamide | ¹H NMR (CDCl₃) δ 8.53 (d, 1H), 8.43 (d, 1H), 8.33 (s, 1H), 8.25 (d, 1H), 7.56 (d, 2H), 7.07 (d, 1H), 6.97 (d, 2H), 3.91-3.87 (m, 4H), 3.16 (m, 4H), 2.85 (m, 1H), 1.6-2.1 (m, 8H). LC-MS [M + H]⁺ 469.2316 |
| 57 | 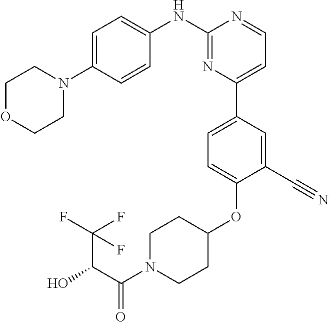 [SMILES: N#Cc1cc(-c2ccnc(Nc3ccc(N4CCOCC4)cc3)n2)ccc1OC1CCN(C(=O)[C@H](O)C(F)(F)F)CC1] | 5-(2-{[4-(Morpholin- 4- yl)phenyl]amino} pyrimidin-4-yl)-2-({1- [(2S)-3,3,3-trifluoro- 2- hydroxypropanoyl] piperidin-4- yl}oxy)benzonitrile | ¹H NMR (DMSO-d₆) δ 9.58 (s, 1H), 8.54-8.53 (m, 1H), 8.51 (d, 1H), 8.47-8.44 (m, 1H), 7.70-7.67 (m, 2H), 7.58-7.55 (m, 1H), 7.43 (d, 1H), 7.03 (d, 2H), 5.20-5.15 (m, 1H), 5.04-5.01 (m, 1H), 3.85-3.71 (m, 6H), 3.67-3.41 (m, 3H), 3.16- 3.12 (m, 4H), 2.09-1.94 (m, 2H), 1.83-1.65 (m, 2H). LC-MS [M + H]⁺ 583.2233 |
| 58 | 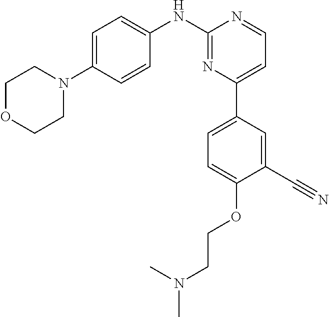 [SMILES: CN(C)CCOc1ccc(-c2ccnc(Nc3ccc(N4CCOCC4)cc3)n2)cc1C#N] | 2-[2- (Dimethylamino) ethoxy]-5-(2-{[4- (morpholin-4- yl)phenyl]amino} pyrimidin-4- yl)benzonitrile | ¹H NMR (CDCl₃) δ 8.43 (d, 1H), 8.29 (d, 1H), 8.24-8.21 (m, 1H), 7.56-7.53 (m, 2H), 7.17 (s, 1H), 7.07 (d, 1H), 7.01 (d, 1H), 6.97- 6.95 (m, 2H), 4.30 (t, 2H), 3.90- 3.87 (m, 4H), 3.16-3.13 (m, 4H), 2.93 (t, 2H), 2.46 (s, 6H). LC-MS [M + H]⁺ 445.2386 |
| 59 | 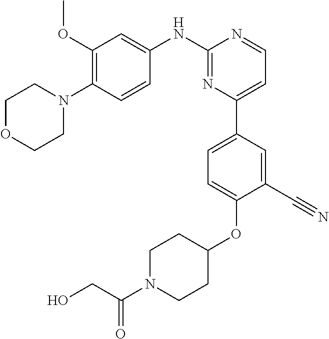 [SMILES: COc1cc(Nc2nccc(-c3ccc(OC4CCN(C(=O)CO)CC4)c(C#N)c3)n2)ccc1N1CCOCC1] | 2-{[1- (hydroxyacetyl) piperidin-4-yl]oxy}- 5-(2-{[3-methoxy-4- (morpholin-4- yl)phenyl]amino} pyrimidin-4- yl)benzonitrile | ¹H NMR (MeOH-d₄) δ 8.57 (d, 1H), 8.54 (d, 1H), 8.42 (dd, 1H), 8.06 (s, 1H), 7.47-7.35 (m, 4H), 4.28 (s, 2H), 4.09 (s, 3H), 4.06- 4.04 (m, 4H), 3.55-3.50 (m, 3H), 3.43-3.40 (m, 4H), 2.90 (s, 2H), 2.15-2.10 (m, 2H), 2.07-2.00 (m, 2H). LC-MS [M + H]⁺ 545.2436 |
| 60 | 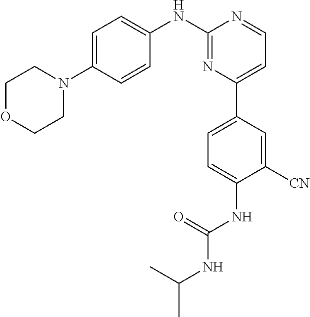 [SMILES: CC(C)NC(=O)Nc1ccc(-c2ccnc(Nc3ccc(N4CCOCC4)cc3)n2)cc1C#N] | 1-[2-cyano-4-(2-{[4- (morpholin-4- yl)phenyl]amino} pyrimidin-4-yl)phenyl]- 3-propan-2-ylurea | ¹H NMR (CDCl₃) δ 8.47 (d, 1H), 8.38 (d, 1H), 8.26 (d, 1H), 8.18 (dd, 1H), 7.58 (d, 2H), 7.045 (d, 1H), 6.98 (dd, 2H), 6.63 (d, 1H), 3.90 (m, 4H), 3.16 (m, 4H), 1.17- 1.28 (m, 7H). LC-MS [M + H]⁺ 458.2281 |
| 61 | 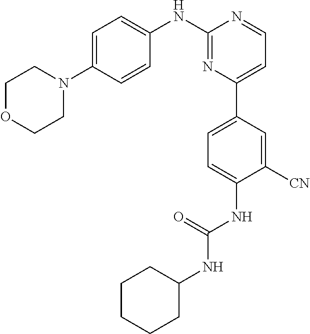 [SMILES: N#Cc1cc(-c2ccnc(Nc3ccc(N4CCOCC4)cc3)n2)ccc1NC(=O)NC1CCCCC1] | 1-[2-cyano-4-(2-{[4- (morpholin-4- yl)phenyl]amino} pyrimidin-4-yl)phenyl]- 3-cyclohexylurea | ¹H NMR (CDCl₃) δ 8.47 (d, 1H), 8.38 (d, 1H), 8.26 (d, 1H), 8.18 (dd, 1H), 7.59 (d, 2H), 7.06 (d, 1H), 6.96-7.00 (m, 2H), 6.72 (d, 1H), 3.90 (m, 4H), 3.63-3.67 (m, 1H), 3.15-3.17 (m, 4H), 1.95-1.99 (m, 2H), 1.73-1.78 (m, 2H), 1.18-1.45 (m, 6H). LC-MS [M + H]⁺ 498.2583 |
| 62 | 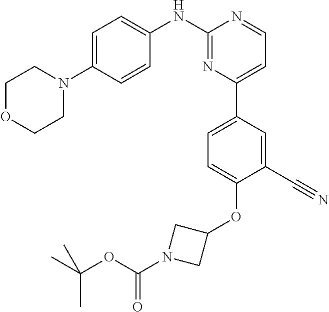 [SMILES: CC(C)(C)OC(=O)N1CC(Oc2ccc(-c3ccnc(Nc4ccc(N5CCOCC5)cc4)n3)cc2C#N)C1] | tert-butyl 3-[2- cyano-4-(2-{[4- (morpholin-4- yl)phenyl]amino} pyrimidin-4- yl)phenoxy]azetidine- 1-carboxylate | ¹H NMR (DMSO-d₆) δ 9.48 (s, 1H), 8.55 (d, 1H), 8.50 (d, 1H), 8.44- 8.41 (m, 1H), 7.64-7.62 (m, 2H), 7.40 (d, 1H), 7.16 (d, 1H), 6.94- 6.91 (m, 2H), 5.27-5.21 (m, 1H), 4.43-4.36 (m, 2H), 3.93-3.87 (m, 2H), 3.76-3.73 (m, 4H), 3.06-3.03 (m, 4H), 1.40 (s, 9H) LC-MS [M + H]⁺ 529.2522. |
| 63 | 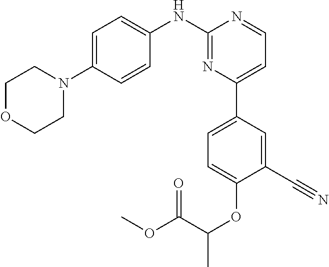 [SMILES: COC(=O)C(C)Oc1ccc(-c2ccnc(Nc3ccc(N4CCOCC4)cc3)n2)cc1C#N] | Methyl 2-[2-cyano-4- (2-{[4-(morpholin-4- yl)phenyl]amino} pyrimidin-4- yl)phenoxy] propanoate | ¹H NMR (CDCl₃) δ 8.44 (d, 1H), 8.32 (d, 1H), 8.20-8.17 (m, 1H), 7.55-7.52 (m, 2H), 7.20 (br s, 1H), 7.01 (d, 1H), 6.97-6.95 (m, 2H), 6.90 (d, 1H), 4.95-4.90 (m, 1H), 3.90-3.87 (m, 4H), 3.79 (s, 3H), 3.16-3.14 (m, 4H), 1.76 (d, 3H). LC-MS [M + H]⁺ 460.1979 |
| 64 | 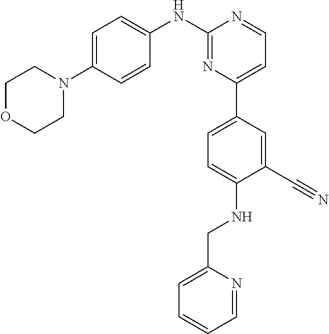 [SMILES: N#Cc1cc(-c2ccnc(Nc3ccc(N4CCOCC4)cc3)n2)ccc1NCc1ccccn1] | 5-(2-{[4-(Morpholin- 4- yl)phenyl]amino} pyrimidin-4-yl)-2- [(pyridin-2- ylmethyl)amino] benzonitrile | ¹H NMR (DMSO-d₆) δ 9.33 (s, 1H), 8.57-8.55 (m, 1H), 8.38 (d, 1H), 8.35 (d, 1H), 8.17-8.14 (m, 1H), 7.80-7.76 (m, 1H), 7.64-7.61 (m, 2H), 7.41 (t, 1H), 7.36 (d, 1H), 7.31-7.28 (m, 1H), 7.24 (d, 1H), 6.93-6.89 (m, 2H), 6.78 (d, 1H), 4.61 (d, 2H), 3.75-3.72 (m, 4H), 3.05-3.02 (m, 4H). LC-MS [M + H]⁺ 464.2259 |
| 65 | 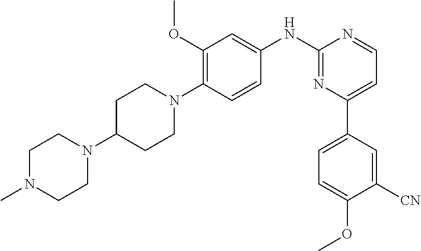 [SMILES: COc1ccc(-c2ccnc(Nc3ccc(N4CCC(N5CCN(C)CC5)CC4)c(OC)c3)n2)cc1C#N] | 2-Methoxy-5-[2-({3- methoxy-4-[4-(4- methylpiperazin-1- yl)piperidin-1- yl]phenyl}amino) pyrimidin-4- yl]benzonitrile | ¹H NMR (MeOH-d₄) δ 8.51-8.48 (m, 2H), 8.40-8.37 (m, 1H), 8.09 (d, 1H), 7.56 (d, 1H), 7.37-7.29 (m, 3H), 4.10 (s, 3H), 4.03 (s, 3H), 3.81-3.72 (m, 2H), 3.69-3.67 (m, 2H), 3.58-3.33 (m, m4H), 3.20-2.90 (m, 5H), 2.92 (s, 3H), 2.27-2.15 (m, 4H). LC-MS [M + H]⁺ 514.4 |
| 66 | 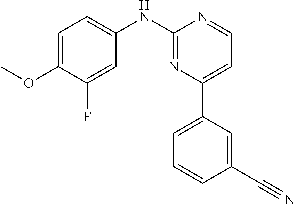 [SMILES: COc1ccc(Nc2nccc(-c3cccc(C#N)c3)n2)cc1F] | 3-{2-[(3-Fluoro-4- methoxyphenyl) amino]pyrimidin-4- yl}benzonitrile | ¹H NMR (CDCl₃) δ 8.51 (d, 1H), 8.35-8.34 (m, 1H), 8.30-8.27 (m, 1H), 7.81-7.78 (m, 1H), 7.67-7.62 (m, 2H), 7.48 (br s, 1H), 7.25-7.22 (m, 1H), 7.16 (d, 1H), 7.00-6.96 (m, 1H), 3.91 (s, 3H). LC-MS [M + H]⁺ 321.1071 |
| 67 | 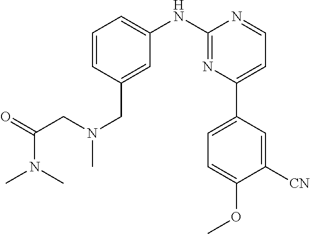 [SMILES: COc1ccc(-c2ccnc(Nc3cccc(CN(C)CC(=O)N(C)C)c3)n2)cc1C#N] | N~2~-(3-{[4-(3- Cyano-4- methoxyphenyl) pyrimidin-2- yl]amino}benzyl)- N,N,N~2~- trimethylglycinamide | ¹H NMR (CDCl₃) δ 8.47 (d, 1H), 8.32-8.29 (m, 2H), 7.66 (s, 1H), 7.62-7.60 (m, 1H), 7.35-7.31 (m, 1H), 7.23 (s, 1H), 7.12-7.07 (m, 3H), 4.03 (s, 3H), 3.63 (s, 2H), 3.26 (s, 2H), 3.04 (s, 3H), 2.92 (s, 3H), 2.35 (s, 3H). LC-MS [M + H]⁺ 431.2188 |
| 68 | 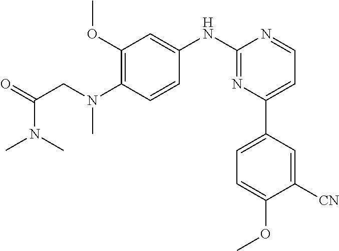 [SMILES: COc1ccc(-c2ccnc(Nc3ccc(N(C)CC(=O)N(C)C)c(OC)c3)n2)cc1C#N] | N~2~-(4-{[4-(3- Cyano-4- methoxyphenyl) pyrimidin-2-yl]amino}- 2-methoxyphenyl)- N,N,N~2~- trimethylglycinamide | ¹H NMR (CDCl₃) δ 8.444 (d, 1H), 8.37 (d, 1H), 8.26-8.23 (m, 1H), 7.56 (bs, 1H), 7.09-7.04 (m, 3H), 6.98 (bs, 1H), 4.02 (s, 3H), 4.01 (s, 2H), 3.95 (s, 3H), 3.05 (s, 3H), 2.95 (s, 3H), 2.93 (s, 3H). LC-MS [M + H]⁺ 447.2140 |
| 69 | 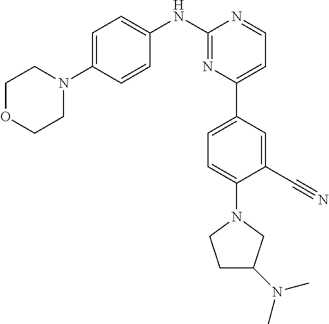 [SMILES: CN(C)C1CCN(c2ccc(-c3ccnc(Nc4ccc(N5CCOCC5)cc4)n3)cc2C#N)C1] | 2-[3- (Dimethylamino) pyrrolidin-1-yl- {5-(2-{[4- (morpholin-4- yl)phenyl]amino} pyrimidin-4- yl)benzonitrile | ¹H NMR (DMSO-d₆) δ 9.46 (br s, 1H), 8.44-8.40 (m, 2H), 8.28-8.24 (m, 1H), 7.65 (d, 2H), 7.36 (d, 1H), 7.01-6.94 (m, 3H), 4.04-3.82 (m, 3H), 3.86-3.74 (m, 6H), 3.09-3.07 (m, 4H), 2.89 (s, 3H), 2.88 (s, 3H), 2.26-2.20 (m, 2H). LC-MS [M + H]⁺ 470.270 |
| 70 | 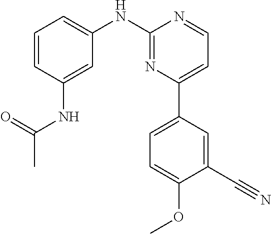 [SMILES: COc1ccc(-c2ccnc(Nc3cccc(NC(C)=O)c3)n2)cc1C#N] | N-(3-{[4-(3-Cyano- 4- methoxyphenyl) pyrimidin-2- yl]amino}phenyl) acetamide | ¹H NMR (DMSO-d₆) δ 9.92 (s, 1H), 9.69 (s, 1H), 8.64 (d, 1H), 8.55- 8.52 (m, 2H), 8.38 (s, 1H), 7.47 (d, 1H), 7.40 (d, 1H), 7.32-7.30 (m, 1H), 7.22-7.17 (m, 1H), 7.11-7.09 (m, 1H), 4.02 (s, 3H), 2.07 (s, 3H). LC-MS [M + H]⁺ 360.1676 |
| 71 | 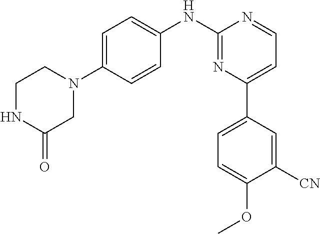 [SMILES: COc1ccc(-c2ccnc(Nc3ccc(N4CCNC(=O)C4)cc3)n2)cc1C#N] | 2-Methoxy-5-(2-{[4- (3-oxopiperazin-1- yl)phenyl]amino} pyrimidin-4- yl)benzonitrile | ¹H NMR (CDCl₃) δ 8.41 (d, 1H), 8.31-8.28 (m, 2H), 7.60 (d, 2H), 7.15-7.13 (m, 1H), 7.07 (d, 1H), 6.96 (*d, 2H), 4.03 (s, 3H), 3.85 (d, 2H), 3.53-3.50 (m, 2H), 3.46-=3.44 (m, 2H). LC-MS [M + H]⁺ 401.1703 |
| 72 | 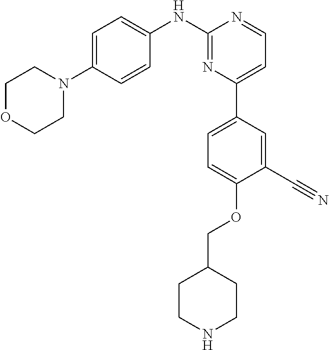 [SMILES: N#Cc1cc(-c2ccnc(Nc3ccc(N4CCOCC4)cc3)n2)ccc1OCC1CCNCC1] | 5-(2-{[4-(morpholin- 4- yl)phenyl]amino} pyrimidin-4-yl)-2- (piperidin-4- ylmethoxy) benzonitrile | ¹H NMR (CDCl₃) δ 8.43 (d, 1H), 8.29 (d, 1H), 8.23-8.21 (m, 1H), 7.56-7.53 (m, 2H), 7.15 (s, 1H), 7.06-6.94 (m, 4H), 3.95 (d, 2H), 3.90-3.87 (m, 4H), 3.18-3.13 (m, 6H), 2.72-2.64 (m, 2H), 2.12-2.02 (m,, 1H), 1.94-1.87 (m, 2H), 1.36- 1.25 (m, 2H). LC-MS [M + H]⁺ 471.2403 |
| 73 | 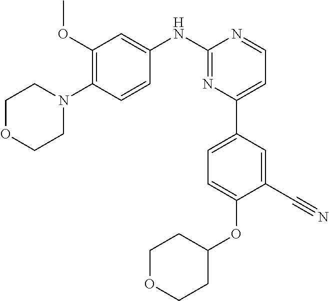 [SMILES: COc1cc(Nc2nccc(-c3ccc(OC4CCOCC4)c(C#N)c3)n2)ccc1N1CCOCC1] | 5-(2-{[3-methoxy-4- (morpholin-4- yl)phenyl]amino} pyrimidin-4-yl)-2- (tetrahydro-2H- pyran-4- yloxy)benzonitrile | ¹H NMR (DMSO-d₆) δ 9.74 (s, 1H), 8.58-8.55 (m, 2H), 8.46-8.43 (m, 1H), 7.77 (d, 1H), 7.57-7.44 (m, 2H), 7.32-7.29 (m, 1H), 7.14-7.06 (m, 1H), 4.98-4.92 (m, 1H), 3.89- 3.71 (m, 9H), 3.59-3.52 (m, 2H), 3.14 (apparent s, 4H), 2.05-2.02 (m, 2H), 1.73-1.64 (m, 2H). Shown as a mixture of rotamers LC-MS [M + H]⁺ 488.2276 |
| 74 | 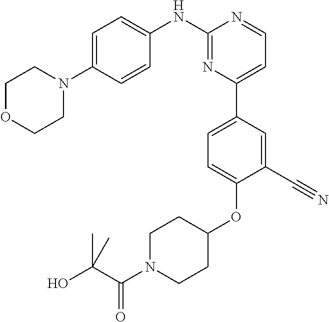 [SMILES: CC(C)(O)C(=O)N1CCC(Oc2ccc(-c3ccnc(Nc4ccc(N5CCOCC5)cc4)n3)cc2C#N)CC1] | 2-{[1-(2-Hydroxy-2- methylpropanoyl) piperidin-4-yl]oxy}-5- (2-{[4-(morpholin-4- yl)phenyl]amino} pyrimidin-4- yl)benzonitrile | ¹H NMR (DMSO-d₆) δ 9.47 (s, 1H), 8.52-8.43 (m, 3H), 7.64 (d, 2H), 7.56 (d, 1H), 7.40 (d, 1H), 6.93 (d, 2H), 5.02-4.98 (m, 1H), 3.81 (br s, 1H), 3.76-3.73 (m, 4H), 3.38-3.32 (m, 4H), 3.06-3.03 (m, 4H), 2.00 (br s, 2H), 1.70 (br s, 2H), 1.33 (s, 6H). LC-MS [M + H]⁺ 543.2632 |
| 75 | 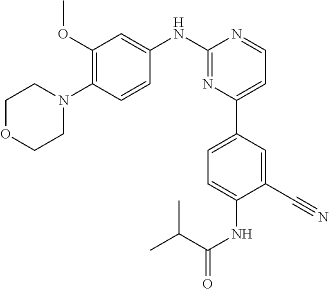 [SMILES: COc1cc(Nc2nccc(-c3ccc(NC(=O)C(C)C)c(C#N)c3)n2)ccc1N1CCOCC1] | N-[2-cyano-4-(2-{[3- methoxy-4- (morpholin-4- yl)phenyl]amino} pyrimidin-4-yl)phenyl]- 2- methylpropanamide | LC-MS [M + H]⁺ 473.2314 |
| 76 | 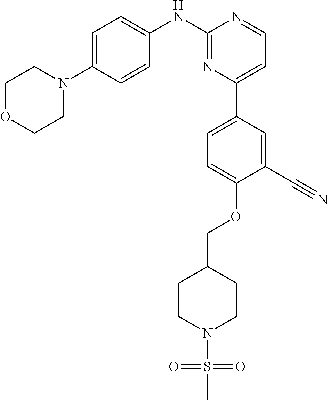 [SMILES: CS(=O)(=O)N1CCC(COc2ccc(-c3ccnc(Nc4ccc(N5CCOCC5)cc4)n3)cc2C#N)CC1] | 2-{[1- (methylsulfonyl) piperidin-4-yl]methoxy}- 5-(2-{[4-(morpholin- 4- yl)phenyl]amino} pyrimidin-4- yl)benzonitrile | ¹H NMR (DMSO-d₆) δ 9.64 (s, 1H), 8.52-8.44 (m, 3H), 7.65-7.62 (m, 2H), 7.44 (d, 1H), 7.39 (d, 1H), 6.92 (d, 2H), 4.15 (d, 2H), 3.76- 3.73 (m, 4H), 3.64-3.59 (m, 2H), 3.06-3.03 (m, 4H), 2.87 (s, 3H), 2.80-2.74 (m, 2H), 2.04-1.87 (m, 3H), 1.46-1.35 (m, 2H). LC-MS [M + H]⁺ 549.2338 |
| 77 | 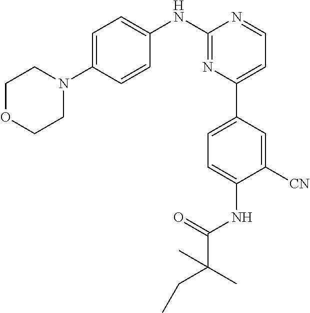 [SMILES: CCC(C)(C)C(=O)Nc1ccc(-c2ccnc(Nc3ccc(N4CCOCC4)cc3)n2)cc1C#N] | N-[2-cyano-4-(2-{[4- (morpholin-4- yl)phenyl]amino} pyrimidin-4-yl)phenyl]- 2,2- dimethylbutanamide | ¹H NMR (CDCl₃) δ 8.63 (d, 1H), 8.46 (d, 1H), 8.33 (s, 1H), 8.26- 8.23 (m, 1H), 8.08 (s, 1H), 7.56- 7.52 (m, 2H), 7.15 (s, 1H), 7.06 (d, 1H), 6.98-6.94 (m, 2H), 3.90-3.88 (m, 4H), 3.17-3.14 (m, 4H), 1.75- 1.69 (m, 2H), 1.35 (s, 6H), 0.98- 0.94 (m, 3H). LC-MS [M + H]⁺ 471.2473 |
| 78 | 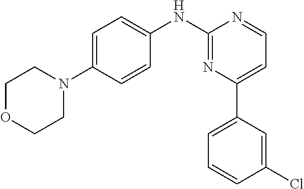 [SMILES: Clc1cccc(-c2ccnc(Nc3ccc(N4CCOCC4)cc3)n2)c1] | 4-(3-Chlorophenyl)- N-[4-(morpholin-4- yl)phenyl]pyrimidin- 2-amine | ¹H NMR (DMSO-d₆) δ 9.54 (s, 1H), 8.53 (d, 1H), 8.22-8.20 (m, 1H), 8.13-8.10 (m, 1H), 7.67-7.56 (m, 4H), 7.40 (d, 1H), 6.96 (d, 2H), 3.77-3.74 (m, 4H), 3.08-3.06 (m, 4H). LC-MS [M + H]⁺ 367.1316 |
| 79 | 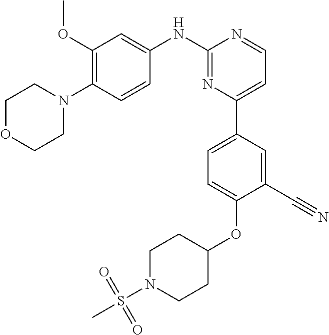 [SMILES: COc1cc(Nc2nccc(-c3ccc(OC4CCN(S(C)(=O)=O)CC4)c(C#N)c3)n2)ccc1N1CCOCC1] | 5-(2-{[3-methoxy-4- (morpholin-4- yl)phenyl]amino} pyrimidin-4-yl)-2-{[1- (methylsulfonyl) piperidin-4- yl]oxy}benzonitrile | ¹H NMR (MeOH-d₄) δ 8.60 (d, 1H), 8.53 (d, 1H), 8.42 (dd, 1H), 8.02 (s, 1H), 7.43-7.33 (m, 4H), 4.08 (s, 3H), 4.04-4.02 (m, 4H), 3.55-3.50 (m, 4H), 3.43-3.40 (m, 4H), 2.90 (s, 3H), 2.15-2.10 (m, 3H), 2.07-2.00 (m, 2H). LC-MS [M + H]⁺ 565.2220 |
| 80 | 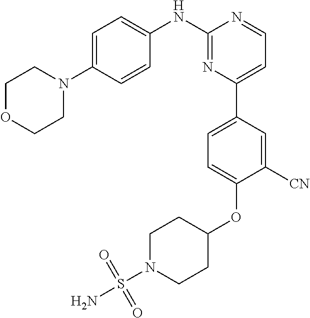 [SMILES: N#Cc1cc(-c2ccnc(Nc3ccc(N4CCOCC4)cc3)n2)ccc1OC1CCN(S(N)(=O)=O)CC1] | 4-[2-cyano-4-(2-{[4- (morpholin-4- yl)phenyl]amino} pyrimidin-4- yl)phenoxy]piperidine- 1-sulfonamide | ¹H NMR (DMSO-d₆) δ 9.46 (s, 1H), 8.45 (m, 3H), 7.63 (d, 2H), 7.54 (d, 1H), 7.39 (d, 1H), 6.91 (m, 4H), 4.86 (m, 1H), 3.75 (m, 4H), 3.26 (m, 2H), 3.05 (m, 6H), 2.06 (m, 2H), 1.86 (m, 2H). LC-MS [M + H]⁺ 536.2057 |
| 81 | 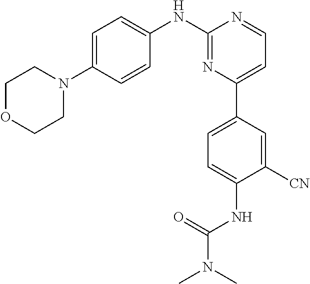 [SMILES: CN(C)C(=O)Nc1ccc(-c2ccnc(Nc3ccc(N4CCOCC4)cc3)n2)cc1C#N] | 3-[2-cyano-4-(2-{[4- (morpholin-4- yl)phenyl]amino} pyrimidin-4-yl)phenyl]- 1,1-dimethylurea | ¹H NMR (CDCl₃) δ 8.49 (d, 1H), 8.43 (d, 1H), 8.29 (d, 1H), 8.21 (dd, 1H), 7.55 (dd, 2H), 7.18 (s, 1H), 7.11 (s, 1H), 7.04 (d, 1H), 6.96 (dd, 2H), 3.89 (m, 4H), 3.15 (m, 4H), 3.13 (s, 6H). LC-MS [M + H]⁺ 444.2145 |
| 82 | 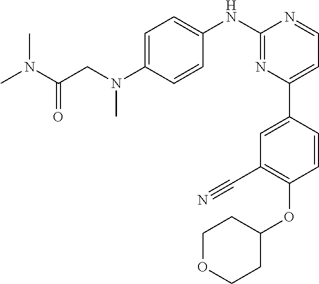 [SMILES: CN(C)C(=O)CN(C)c1ccc(Nc2nccc(-c3ccc(OC4CCOCC4)c(C#N)c3)n2)cc1] | N~2~-[4-({4-[3- cyano-4-(tetrahydro- 2H-pyran-4- yloxy)phenyl] pyrimidin-2- yl}amino)phenyl]- N,N,N~2~- trimethylglycinamide | LC-MS [M + H]⁺ 487.2492 |
| 83 | 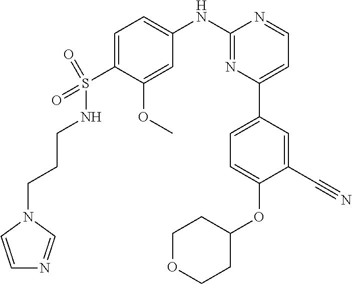 [SMILES: COc1cc(Nc2nccc(-c3ccc(OC4CCOCC4)c(C#N)c3)n2)ccc1S(=O)(=O)NCCCn1ccnc1] | 4-({4-[3-cyano-4- (tetrahydro-2H- pyran-4- yloxy)phenyl] pyrimidin-2-yl}amino)-N- [3-(1H-imidazol-1- yl)propyl]-2- methoxybenzene- sulfonamide | ¹H NMR (CDCl₃) δ 8.54 (d, 1H); 8.39 (d, 1H); 8.21 (d, 1H); 7.98 (s, 1H) 7.82 (d, 1H); 7.67 (s, 1H); 7.50 (s, 1H); 7.20 (d, 1H); 7.13-7.05 (m, 3H); 6.93 (s, 1H); 5.04 (t, 1H); 4.79-4.76 (m, 1H), 4.12-4.02 (m, 7H); 3.70-3.65 (m, 2H); 2.84 (q, 2H); 2.13-2.08 (m, 2H); 2.00-1.90 (m, 4H). LC-MS [M + H]⁺ 590.2225 |
| 84 | 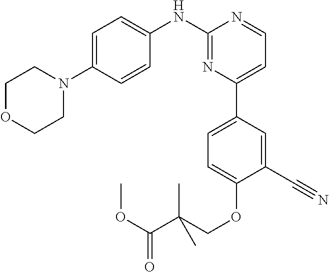 [SMILES: COC(=O)C(C)(C)COc1ccc(-c2ccnc(Nc3ccc(N4CCOCC4)cc3)n2)cc1C#N] | methyl 3-[2-cyano-4- (2-{[4-(morpholin-4- yl)phenyl]amino} pyrimidin-4- yl)phenoxy]-2,2- dimethylpropanoate | ¹H NMR (DMSO-d₆) δ 9.47 (s, 1H), 8.51-8.44 (m, 3H), 7.65-7.62 (m, 2H), 7.46 (d, 1H), 7.39 (d, 1H), 6.94-6.91 (m, 2H), 4.26 (s, 2H), 3.76-3.74 (m, 4H), 3.64 (s, 3H), 3.06-3.03 (m, 4H), 1.30 (s, 6H). LC-MS [M + H]⁺ 488.2297 |
| 85 | 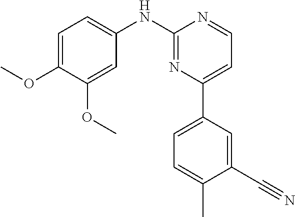 [SMILES: COc1ccc(Nc2nccc(-c3ccc(C)c(C#N)c3)n2)cc1OC] | 5-{2-[(3,4- Dimethoxyphenyl) amino]pyrimidin-4-yl}- 2-methylbenzonitrile | ¹H NMR (DMSO-d₆) δ 9.57 (s, 1H), 8.57-8.55 (m, 2H), 8.40-8.37 (m, 1H), 7.69 (s, 1H), 7.65 (d, 1H), 7.46 (d, 1H), 7.22-7.19 (m, 1H), 6.91 (d, 1H), 3.81 (s, 3H), 3.73 (s, 3H), 2.57 (s, 3H). LC-MS [M + H]⁺ 347.1418 |
| 86 | 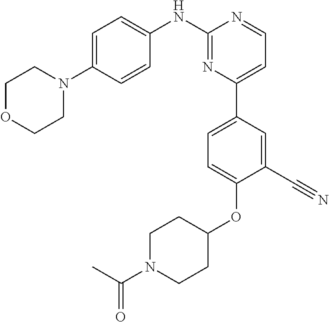 [SMILES: CC(=O)N1CCC(Oc2ccc(-c3ccnc(Nc4ccc(N5CCOCC5)cc4)n3)cc2C#N)CC1] | 2-[(1- Acetylpiperidin-4- yl)oxy]-5-(2-{[4- (morpholin-4- yl)phenyl]amino} pyrimidin-4- yl)benzonitrile | ¹H NMR (CDCl₃) δ 8.45 (d, 1H), 8.31 (d, 1H), 8.25-8.22 (d, 1H), 7.56-7.53 (m, 2H), 7.22 (br s, 1H), 7.08 (d, 1H), 7.12 (d, 1H), 6.97- 6.95 (m, 3H), 4.85-4.80 (m, 1H), 3.98-3.91 (m, 1H), 3.90-3.85 (m, 4H), 3.80-3.73 (m, 1H), 3.64-3.51 (m, 2H), 3.16-3.13 (m, 4H), 2.14 (s, 3H), 2.02-1.92 (m, 4H). LC-MS [M + H]⁺ 499.2347 |
| 87 | 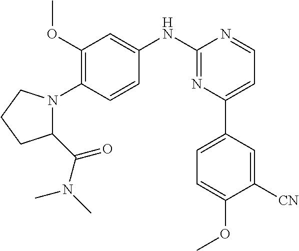 [SMILES: COc1ccc(-c2ccnc(Nc3ccc(N4CCCC4C(=O)N(C)C)c(OC)c3)n2)cc1C#N] | 1-(4-{[4-(3-Cyano-4- methoxyphenyl) pyrimidin-2-yl]amino}- 2-methoxyphenyl)- N,N- dimethylprolinamide] | ¹H NMR (CDCl₃) δ 8.41-8.36 (m, 2H), 8.22-8.20 (m, 1H), 7.47 (bs, 1H), 7.05 (d, 1H), 6.98 (d, 1H), 6.91 (d, 1H), 6.79 (d, 1H), 5.05- 5.02 (m, 1H), 3.99 (s, 3H), 3.84 (s, 3H), 3.74-3.69 (m, 1H), 3.34-3.29 (m, 1H), 3.08 (s, 3H), 2.91 (s, 3H), 2.32-2.29 (m, 1H), 2.14-2.05 (m, 1H), 1.99-1.88 (m, 2H). LC-MS [M + H]⁺ 473.2313 |
| 88 | 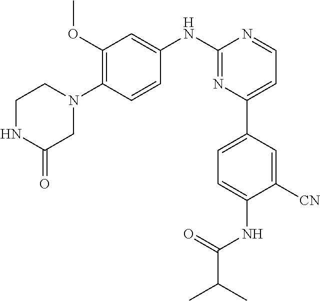 [SMILES: COc1cc(Nc2nccc(-c3ccc(NC(=O)C(C)C)c(C#N)c3)n2)ccc1N1CCNC(=O)C1] | N-[2-Cyano-4-(2- {[3-methoxy-4-(3- oxopiperazin-1- yl)phenyl]amino} pyrimidin-4-yl)phenyl]- 2- methylpropanamide | ¹H NMR (CDCl₃) δ 8.50 (s, 1H), 8.47 (d, 1H), 8.40 (d, 1H), 8.27- 8.24 (m, 1H), 7.61 (bs, 1H), 7.32- 7.07 (m, 2H), 6.93 (d, 1H), 3.97 (s, 3H), 3.78 (s, 2H), 3.51-3.48 (m, 2H), 3.34-3.31 (m, 2H), 2.74-2.67 (m, 1H), 1.32 (d, 6H). LC-MS [M + H]⁺ 486.2245 |
| 89 | 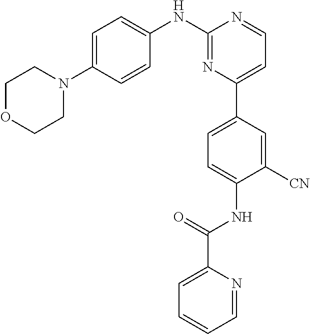 [SMILES: N#Cc1cc(-c2ccnc(Nc3ccc(N4CCOCC4)cc3)n2)ccc1NC(=O)c1ccccn1] | N-[2-cyano-4-(2-{[4- (morpholin-4- yl)phenyl]amino} pyrimidin-4- yl)phenyl]pyridine-2- carboxamide | ¹H NMR (CDCl₃) δ 8.87 (d, 1H), 8.73 (m, 1H), 8.47 (d, 1H), 8.40 (d, 1H), 8.33 (m, 2H), 7.96 (m, 1H), 7.59 (m, 3H), 7.09 (m, 2H), 6.97 (m, 2H), 3.90 (m, 4H), 3.49 (d, 1H) 3.16 (m, 4H). LC-MS [M + H]⁺ 478.1971 |
| 90 | 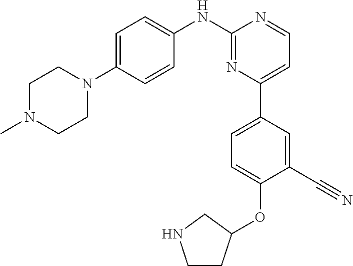 [SMILES: CN1CCN(c2ccc(Nc3nccc(-c4ccc(OC5CCNC5)c(C#N)c4)n3)cc2)CC1] | 5-(2-{[4-(4- methylpiperazin-1- yl)phenyl]amino} pyrimidin-4-yl)-2- (pyrrolidin-3- yloxy)benzonitrile | LC-MS [M + H]⁺ 456.30 |
| 91 | 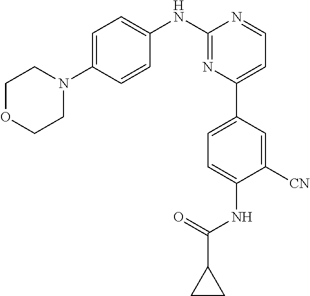 [SMILES: N#Cc1cc(-c2ccnc(Nc3ccc(N4CCOCC4)cc3)n2)ccc1NC(=O)C1CC1] | N-[2-cyano-4-(2-{[4- (morpholin-4- yl)phenyl]amino} pyrimidin-4- yl)phenyl] cyclopropane- carboxamide | ¹H NMR (CDCl₃) δ 8.49 (d, 1H), 8.43 (d, 1H), 8.33 (s, 1H), 8.22 (d, 1H), 7.57 (d, 2H), 7.06 (d, 1H), 6.97 (d, 2H), 3.91-3.87 (m, 4H), 3.18-3.13 (m, 4H), 1.79-1.74 (m, 1H), 1.19-1.13 (m, 2H), 1.01-0.97 (m, 2H). LC-MS [M + H]⁺ 441.1999 |
| 92 | 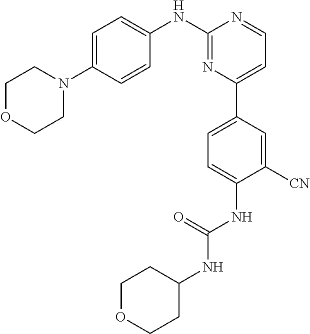 [SMILES: N#Cc1cc(-c2ccnc(Nc3ccc(N4CCOCC4)cc3)n2)ccc1NC(=O)NC1CCOCC1] | 1-[2-cyano-4-(2-{[4- (morpholin-4- yl)phenyl]amino} pyrimidin-4-yl)phenyl]- 3-(tetrahydro-2H- pyran-4-yl)urea | ¹H NMR (CDCl₃) δ 8.49 (d, 1H), 8.40 (d, 1H), 8.26 (d, 1H), 8.18 (dd, 1H), 7.56 (dd, 2H), 7.04 (d, 1H), 6.97 (dd, 2H), 3.97 (m, 2H), 3.89 (m, 1H), 3.89 (m, 4H), 3.54 (m, 2H), 3.15 (m, 4H), 1.97 (m, 2H), 1.55 (m, 2H). LC-MS [M + H]⁺ 500.2831 |
| 93 | 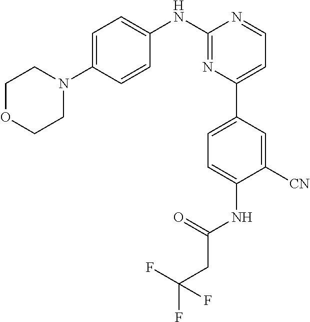 [SMILES: N#Cc1cc(-c2ccnc(Nc3ccc(N4CCOCC4)cc3)n2)ccc1NC(=O)CC(F)(F)F] | N-[2-cyano-4-(2-{[4- (morpholin-4- yl)phenyl]amino} pyrimidin-4-yl)phenyl]- 3,3,3- trifluoropropanamide | ¹H NMR (CDCl₃) δ 8.44 (d, 1H), 8.37 (dd, 1H), 8.27 (d, 2H), 7.58-7.56 (m, 2H), 7.09 (d, 1H), 6.99-6.97 (m, 2H), 3.91-3.89 (m, 4H), 3.44-3.41 (m, 2H), 3.18-3.15 (m, 4H). LC-MS [M + H]⁺ 483.1733 |
| 94 | 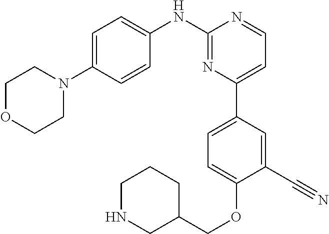 [SMILES: N#Cc1cc(-c2ccnc(Nc3ccc(N4CCOCC4)cc3)n2)ccc1OCC1CCCNC1] | 5-(2-{[4-(morpholin- 4- yl)phenyl]amino} pyrimidin-4-yl)-2- (piperidin-3- ylmethoxy) benzonitrile] | ¹H NMR (DMSO-d₆) δ 9.49 (s, 1H), 8.95-8.80 (m, 1H), 8.75-8.68 (m, 1H), 8.54-8.46 (m, 3H), 7.64 (d, 2H), 7.45 (d, 1H), 7.40 (d, 1H), 6.97-6.91 (m, 2H), 4.26-4.11 (m, 2H), 3.77-3.74 (m, 4H), 3.56-3.26 (m, 4H), 3.09-3.01 (m, 4H), 2.84- 2.75 (m, 2H), 2.33 (br s, 1H), 1.92- 1.82 (m, 2H), 1.72-1.67 (m, 1H), 1.42-1.32 (m, 1H). LC-MS [M + H]⁺ 471.2384 |
| 95 | 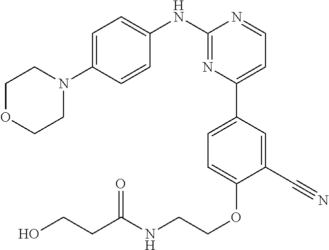 [SMILES: N#Cc1cc(-c2ccnc(Nc3ccc(N4CCOCC4)cc3)n2)ccc1OCCNC(=O)CCO] | N-{2-[2-cyano-4-(2- {[4-(morpholin-4- yl)phenyl]amino} pyrimidin-4- yl)phenoxy]ethyl}-3- hydroxypropanamide] | ¹H NMR (DMSO-d₆) δ 9.46 (s, 1H), 8.52-8.43 (m, 3H), 8.16-8.14 (m, 1H), 7.63 (d, 2H), 7.46 (d, 1H), 7.40 (d, 1H), 6.93 (d, 2H), 4.63- 4.58 (m, 1H), 4.28-4.25 (m, m2H), 3.76-3.73 (m, 4H), 3.64-3.59 (m, 2H), 3.51-3.46 (m, 2H), 3.06-3.03 (m, 4H), 2.27 (t, 2H). LC-MS [M + H]⁺ 489.2228 |
| 96 | 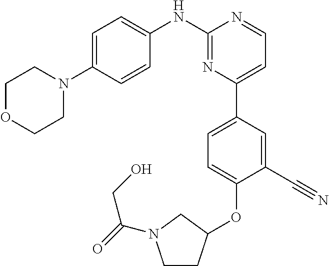 [SMILES: N#Cc1cc(-c2ccnc(Nc3ccc(N4CCOCC4)cc3)n2)ccc1OC1CCN(C(=O)CO)C1] | 2-{[1- (Hydroxyacetyl) pyrrolidin- 3-yl]oxy}-5- (2-{[4-(morpholin-4- yl)phenyl]amino} pyrimidin-4- yl)benzonitrile | ¹H NMR (MeOH-d₄) δ 8.47-8.41 (m, 4H), 7.88-7.74 (m, 2H), 7.48- 7.32 (m, 4H), 5.38-5.32 (m, 2H), 4.25-4.18 (m, 3H), 4.08-4.95 (m, 5H), 3.88-3.48 (m, 8H), 2.40-2.27 (m, 4H). Rotamers. LC-MS [M + H]⁺ 501.2328 |
| 97 | 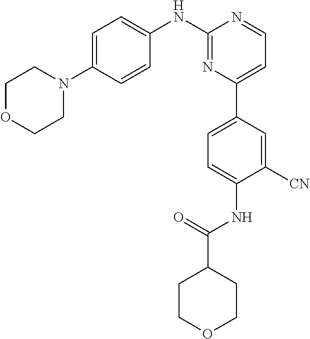 [SMILES: N#Cc1cc(-c2ccnc(Nc3ccc(N4CCOCC4)cc3)n2)ccc1NC(=O)C1CCOCC1] | N-[2-cyano-4-(2-{[4- (morpholin-4- yl)phenyl]amino} pyrimidin-4- yl)phenyl]tetrahydro- 2H-pyran-4- carboxamide | ¹H NMR (CDCl₃) δ 8.62 (d, 1H), 8.46 (d, 1H), 8.35 (d, 1H), 8.26 (dd, 1H), 7.79 (s, 1H), 7.55 (dd, 2H), 7.06 (d, 2H), 6.97 (dd, 2H), 4.09 (m, 2H), 3.89 (m, 4H), 3.51 (m, 2H), 3.15 (m, 4H), 2.64 (m, 1H), 1.94 (m, 4H). LC-MS [M + H]⁺ 485.2274 |
| 98 | 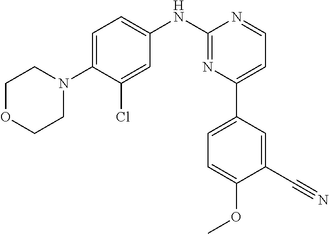 [SMILES: COc1ccc(-c2ccnc(Nc3ccc(N4CCOCC4)c(Cl)c3)n2)cc1C#N] | 5-(2-{[3-Chloro-4- (morpholin-4- yl)phenyl]amino} pyrimidin-4-yl)-2- methoxybenzonitrile] | ¹H NMR (DMSO-d₆) δ 9.78 (s, 1H), 8.57-8.47 (m, 3H), 8.06 (d, 1H), 7.67-7.63 (m, 1H), 7.49 (d, 1H), 7.44 (d, 1H), 7.15 (d, 1H), 4.02 (s, 3H), 3.76-3.73 (m, 4H), 2.94-2.92 (m, 4H). LC-MS [M + H]⁺ 422.1388 |
| 99 | 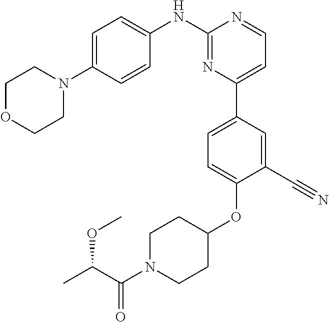 [SMILES: CO[C@@H](C)C(=O)N1CCC(Oc2ccc(-c3ccnc(Nc4ccc(N5CCOCC5)cc4)n3)cc2C#N)CC1] | 2-({1-[(2S)-2- methoxypropanoyl] piperidin-4-yl}oxy)-5- (2-{[4-(morpholin-4- yl)phenyl]amino} pyrimidin-4- yl)benzonitrile] | ¹H NMR (DMSO-d₆) δ 9.71 (s, 1H), 8.56-8.52 (m, 2H), 8.47-8.44 (m, 1H), 7.74 (d, 2H), 7.56 (d, 1H), 7.46 (d, 1H), 7.16 (d, 2H), 5.04- 4.98 (m, 1H), 4.28-4.23 (m, 1H), 3.86-3.74 (m, 6H), 3.56-3.40 (m, 2H), 3.22 (br s, 7H), 2.10-1.92 (m, 2H), 1.79-1.63 (m, 2H), 1.24 (d, 3H). LC-MS [M + H]⁺ 543.2709 |
| 100 | 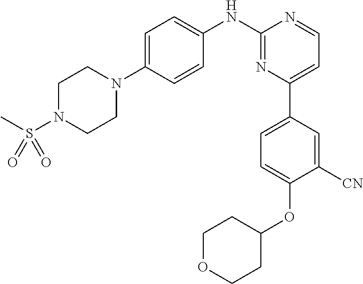 [SMILES: CS(=O)(=O)N1CCN(c2ccc(Nc3nccc(-c4ccc(OC5CCOCC5)c(C#N)c4)n3)cc2)CC1] | 5-[2-({4-[4- (methylsulfonyl) piperazin-1- yl]phenyl}amino) pyrimidin-4-yl]-2- (tetrahydro-2H- pyran-4- yloxy)benzonitrile | ¹H NMR (CDCl₃) δ 8.42 (d, 1H), 8.32 (d, 1H), 8.24 (dd, 1H), 7.59 (d, 2H), 7.12 (d, 1H), 7.06 (d, 1H), 6.99 (dd, 2H), 4.77 (m, 1H), 4.04 (m, 2H), 3.68 (m, 2H), 3.41 (m, 4H), 3.28 (m, 4H), 2.10 (m, 2H), 1.93 (m, 2H). LC-MS [M + H]⁺ 535.2097 |
| 101 | 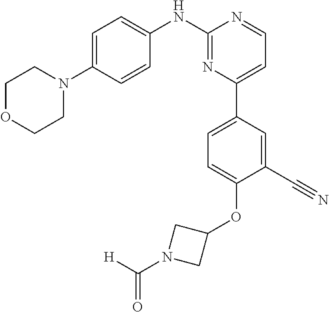 [SMILES: N#Cc1cc(-c2ccnc(Nc3ccc(N4CCOCC4)cc3)n2)ccc1OC1CN(C=O)C1] | 2-[(1-formylazetidin- 3-yl)oxy]-5-(2-{[4- (morpholin-4- yl)phenyl]amino} pyrimidin-4- yl)benzonitrile | ¹H NMR (DMSO-d₆) δ 9.48 (s, 1H), 8.56 (d, 1H), 8.50 (d, 1H), 8.46- 8.42 (m, 1H), 8.06 (s, 1H), 7.64- 7.61 (m, 2H), 7.40 (d, 1H), 7.20 (d, 1H), 6.94-6.91 (m, 2H), 5.40-5.34 (m, 1H), 4.70-4.66 (m, 1H), 4.47- 4.42 (m, 1H), 4.23-4.19 (m, 1H), 3.93-3.89 (m, 1H), 3.76-3.73 (m, 4H), 3.06-3.03 (m, 4H). LC-MS [M + H]⁺ 457.2009 |
| 102 | 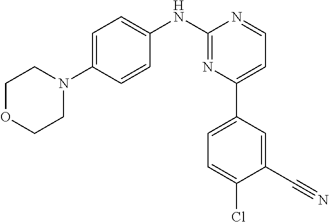 [SMILES: N#Cc1cc(-c2ccnc(Nc3ccc(N4CCOCC4)cc3)n2)ccc1Cl] | 2-Chloro-5-(2-{[4- (morpholin-4- yl)phenyl]amino} pyrimidin-4- yl)benzonitrile | ¹H NMR (DMSO-d₆) δ 9.56 (s, 1H), 8.70 (d, 1H), 8.57 (d, 1H), 8.49- 8.46 (m, 1H), 7.95 (d, 1H), 7.64- 7.61 (m, 2H), 7.47 (d, 1H), 6.94- 6.91 (m, 2H), 3.76-3.73 (m, 4H), 3.06-3.04 (m, 4H). LC-MS [M + H]⁺ 392.1306 |
| 103 | 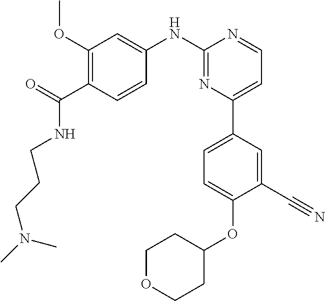 [SMILES: COc1cc(Nc2nccc(-c3ccc(OC4CCOCC4)c(C#N)c3)n2)ccc1C(=O)NCCCN(C)C] | 4-({4-[3-cyano-4- (tetrahydro-2H- pyran-4- yloxy)phenyl] pyrimidin-2-yl} amino)-N-[3- (dimethylamino) propyl]-2- methoxybenzamide | ¹H NMR (DMSO-d₆) δ 10.1 (br s, 1H), 9.58 (br s, 1H), 8.65-8.61 (m, 2H), 8.47 (dd, 1H), 8.30 (t, 1H), 7.96 (s, 1H), 7.83 (d, 1H), 7.60- 7.56 (m, 2H), 7.37 (d, 1H), 4.96 (m, 1H), 4.00 (s, 3H), 3.90-3.85 (m, 2H), 3.67-3.62 (m, 2H), 3.60-3.54 (m, 2H), 3.29-3.24 (m, 2H), 2.85 (s, 6H), 2.08-2.01 (m, 2H), 1.73-1.66 (m, 2H); LC-MS [M + H]⁺ 531.2715 |
| 104 | 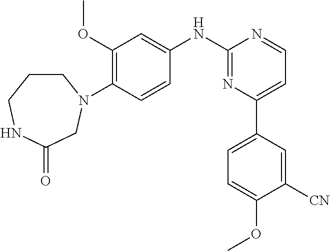 [SMILES: COc1ccc(-c2ccnc(Nc3ccc(N4CCCNC(=O)C4)c(OC)c3)n2)cc1C#N] | 2-Methoxy-5-(2-{[3- methoxy-4-(3-oxo- 1,4-diazepan-1- yl)phenyl]amino} pyrimidin-4- yl)benzonitrile | ¹H NMR (CDCl₃) δ 8.44 (d, 1H), 8.34 (d, 1H), 8.28-8.25 (m, 1H), 7.48 (d, 1H), 7.19 (s, 1H), 7.09- 6.97 (m, 4H), 5.96-5.94 (m, 1H), 4.02 (s, 3H), 3.93 (s, 3H), 3.92 (s, 2H), 3.50-3.48 (m, 2H), 3.41-3.37 (m, 2H), 2.00-1.95 (m, 2H). LC- MS [M + H]⁺ 445.1988 |
| 105 | 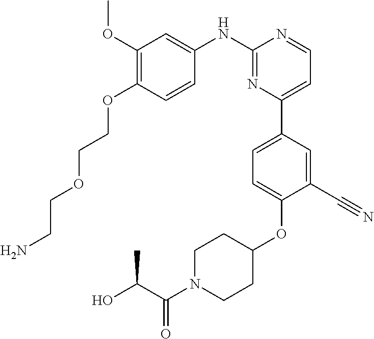 [SMILES: COc1cc(Nc2nccc(-c3ccc(OC4CCN(C(=O)[C@H](C)O)CC4)c(C#N)c3)n2)ccc1OCCOCCN] | 5-[2-({4-[2-(2- aminoethoxy)ethoxy]- 3- methoxyphenyl} amino) pyrimidin-4-yl]-2- ({1-[(2S)-2- hydroxypropanoyl] piperidin-4- yl}oxy)benzonitrile | ¹H NMR (MeOH-d₄) δ 8.54 (d, 1H), 8.44 (d, 1H), 8.40 (dd, 1H), 7.70 (d, 1H), 7.40 (d, 1H), 7.30 (d, 1H), 7.14 (dd, 1H), 7.0 (d, 1H), 4.64-4.60 (m, 1H), 4.20-4.18 (m, 2H), 3.93 (s, 3H), 3.91-3.90 (m, 2H), 3.82-3.80 (m, 3H), 3.71-7.70 (m, 4H), 3.21 (t, 2H), 2.09-2.02 (m, 2H), 1.91-1.81 (m, 2H), 1.34 (d, 3H). LC-MS [M + H]⁺ 577.2656 |
| 106 | 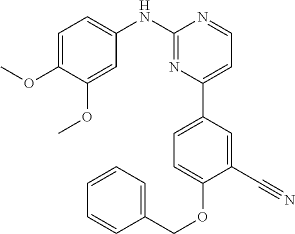 [SMILES: COc1ccc(Nc2nccc(-c3ccc(OCc4ccccc4)c(C#N)c3)n2)cc1OC] | 2-(Benzyloxy)-5-{2- [(3,4- dimethoxyphenyl) amino]pyrimidin-4- yl}benzonitrile | ¹H NMR (CDCl₃) δ 8.37 (d, 1H), 8.25-8.22 (m, 1H), 8.19 (d, 1H), 7.48-7.33 (m, 6H), 7.19-7.14 (m, 3H), 6.91 (d, 1H), 5.34 (s, 2H), 3.93 (s, 3H), 3.92 (s, 3H). LC-MS [M + H]⁺ 439.1798 |
| 107 | 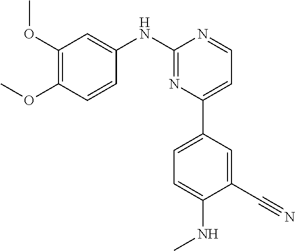 [SMILES: CNc1ccc(-c2ccnc(Nc3ccc(OC)c(OC)c3)n2)cc1C#N] | 5-{2-[(3,4- Dimethoxyphenyl) amino]pyrimidin-4-yl}- 2- (methylamino) benzonitrile | ¹H NMR (CDCl₃) δ 8.38 (d, 1H), 8.22 (d, 1H), 8.16-8.13 (m, 1H), 7.52 (d, 1H), 7.08 (brr s, 1H), 7.03- 6.98 (m, 2H), 6.87 (d, 1H), 6.73 (d, 1H), 4.98-4.95 (m, 1H), 3.96 (s, 3H), 3.89 (s, 3H), 3.01 (d, 3H). LC-MS [M + H]⁺ 362.1665 |
| 108 | AND Enantiomer 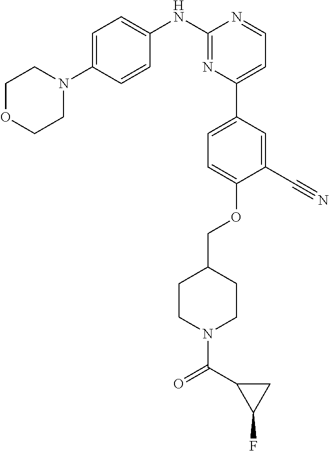 [SMILES: N#Cc1cc(-c2ccnc(Nc3ccc(N4CCOCC4)cc3)n2)ccc1OCC1CCN(C(=O)C2C[C@H]2F)CC1] | 2-[(1-{[(2R)-2- fluorocyclopropyl] carbonyl}piperidin-4- yl)methoxy]-5-(2- {[4-(morpholin-4- yl)phenyl]amino} pyrimidin-4- yl)benzonitrile | ¹H NMR (CDCl₃) δ 8.33-8.22 (m, 3H), 7.63-7.60 (m, 2H), 7.15-7.03 (m, 4H), 4.92-4.67 (m, 2H), 4.29- 4.24 (m, 1H), 4.08-3.99 (m, 2H), 3.94-3.92 (m, 4H), 3.24-3.09 (m, 4H), 2.90-2.45 (m, 4H), 2.30-1.85 (m, 3H), 1.44-1.32 (m, 4H). LC- MS [M + H]⁺ 557.2450 |
| 109 | 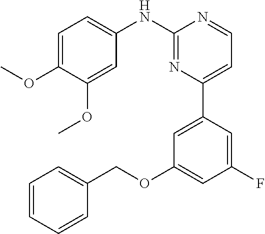 [SMILES: COc1ccc(Nc2nccc(-c3cc(F)cc(OCc4ccccc4)c3)n2)cc1OC] | 4-[3-(Benzyloxy)-5- fluorophenyl]-N- (3,4- dimethoxyphenyl) pyrimidin-2-amine | ¹H NMR (CDCl₃) δ 8.34 (d, 1H), 7.55-7.52 (m, 2H), 7.44-7.36 (m, 6H), 7.10 (d, 1H), 7.08-7.05 (m, 1H), 6.88-6.84 (m, 3H), 5.11 (s, 2H), 3.93 (s, 3H), 3.89 (s, 3H). LC-MS [M + H]⁺ 432.1612 |
| 110 | 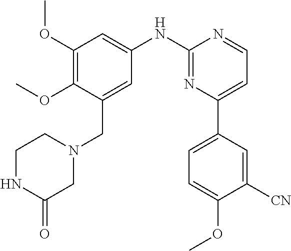 [SMILES: COc1ccc(-c2ccnc(Nc3cc(CN4CCNC(=O)C4)c(OC)c(OC)c3)n2)cc1C#N] | 5-[2-({3,4- Dimethoxy-5-[(3- oxopiperazin-1- yl)methyl]phenyl} amino)pyrimidin-4-yl]- 2- methoxybenzonitrile | ¹H NMR (CDCl₃) δ 8.68 (s, 1H), 8.41 (d, 1H), 8.36-8.29 (m, 3H), 7.50 (d, 1H), 7.32 (d, 1H), 7.13-7.06 (m, 3H), 3.99 (s, 3H), 3.88 (s, 3H), 3.75 (s, 3H), 3.58 (s, 2H), 3.24- 3.21 (m, 2H), 3.15 (s, 2H), 2.65- 2.62 (m, 2H). LC-MS [M + H]⁺ 475.2109 |
| 111 | 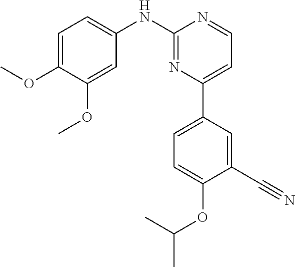 [SMILES: COc1ccc(Nc2nccc(-c3ccc(OC(C)C)c(C#N)c3)n2)cc1OC] | 5-{2-[(3,4- Dimethoxyphenyl) amino]pyrimidin-4-yl}- 2-(propan-2- yloxy)benzonitrile | ¹H NMR (DMSO-d₆) δ 9.51 (s, 1H), 8.53-8.50 (m, 2H), 8.45-8.42 (m, 1H), 7.65 (s, 1H), 7.45 (d, 1H), 7.41 (d, 1H), 7.23-7.20 (m, 1H), 6.91 (d, 1H), 4.96-4.90 (m, 1H), 3.80 (s, 3H), 3.73 (s, 3H), 1.36 (d, 6H). LC-MS [M + H]⁺ 391.1793 |
| 112 | 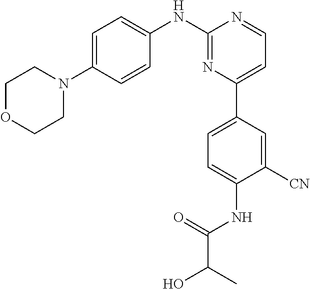 [SMILES: CC(O)C(=O)Nc1ccc(-c2ccnc(Nc3ccc(N4CCOCC4)cc3)n2)cc1C#N] | N-[2-cyano-4-(2-{[4- (morpholin-4- yl)phenyl]amino} pyrimidin-4-yl)phenyl]- 2- hydroxypropanamide | ¹H NMR (CDCl₃) δ 9.03 (s, 1H), 8.51 (dd, 1H), 8.14 (d, 1H), 8.09 (dd, 1H), 7.53 (m, 2H), 7.33 (m, 2H), 6.91 (m, 3H), 5.64 (q, 1H), 3.86 (m, 4H), 3.12 (m, 4H), 1.75 (d, 3H). LC-MS [M + H]⁺ 445.1952 |
| 113 | 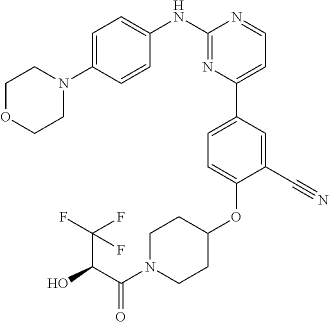 [SMILES: N#Cc1cc(-c2ccnc(Nc3ccc(N4CCOCC4)cc3)n2)ccc1OC1CCN(C(=O)[C@@H](O)C(F)(F)F)CC1] | 5-(2-{[4-(morpholin- 4- yl)phenyl]amino} pyrimidin-4-yl)-2-({1- [(2R)-3,3,3-trifluoro- 2- hydroxypropanoyl] piperidin-4- yl}oxy)benzonitrile | ¹H NMR (DMSO-d₆) δ 9.55 (s, 1H), 8.54-8.43 (m, 3H), 7.67 (d, 2H), 7.58-7.55 (m, 1H), 7.42 (d, 1H), 7.00 (d, 2H), 5.21-5.14 (m, 1), 5.06-4.99 (m, 1H), 3.87-3.71 (m, 6H), 3.66-3.41 (m, 3H), 3.16-3.08 (m, 4H), 2.08-1.66 (m, 4H). LC- MS [M + H]⁺ 583.2259 |
| 114 | 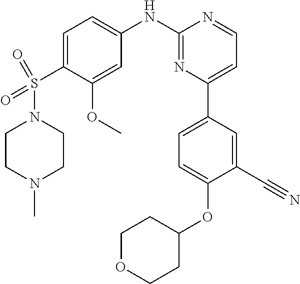 [SMILES: COc1cc(Nc2nccc(-c3ccc(OC4CCOCC4)c(C#N)c3)n2)ccc1S(=O)(=O)N1CCN(C)CC1] | 5-[2-({3-methoxy-4- [(4-methylpiperazin- 1- yl)sulfonyl]phenyl} amino)pyrimidin-4- yl]-2-(tetrahydro-2H- pyran-4- yloxy)benzonitrile | ¹H NMR (DMSO-d₆) δ 10.23 (s, 1H), 8.65 (d, 1H), 8.61 (d, 1H); 8.48 (dd, 1H); 7.99 (s, 1H); 7.65-7.56 (m, 3H); 7.43 (d, 1H); 4.96 (m, 1H); 3.92 (s, 3H); 3.91-3.82 (m, 2H); 3.59-3.35 (m, 2H); 3.052 (m, 4H), 2.32 (m, 4H); 2.15 (s, 3H); 2.07-2.03 (m, 2H); 1.73-1.66 (m, 2H). LC-MS [M + H]⁺ 565.2169 |
| 115 | 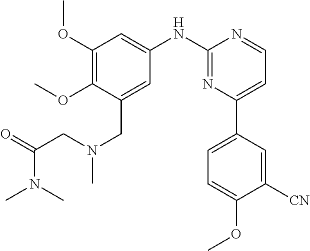 [SMILES: COc1ccc(-c2ccnc(Nc3cc(CN(C)CC(=O)N(C)C)c(OC)c(OC)c3)n2)cc1C#N] | N~2~-(5-{[4-(3- Cyano-4- methoxyphenyl) pyrimidin-2-yl]amino}- 2,3- dimethoxybenzyl)- N,N,N~2~- trimethylglycinamide | ¹H NMR (CDCl₃) δ 8.46 (d, 1H), 8.37 (d, 1H), 8.28-8.25 (m, 1H), 7.61 (bs, 1H), 7.24 (s, 1H), 7.09- 7.05 (m, 3H), 4.02 (s, 3H), 3.95 (s, 3H), 3.80 (s, 3H), 3.65 (s, 2H), 3.28 (s, 2H), 3.04 (s, 3H), 2.93 (s, 3H), 2.35 (s, 3H). LC-MS [M + H]⁺ 491.2413 |
| 116 | 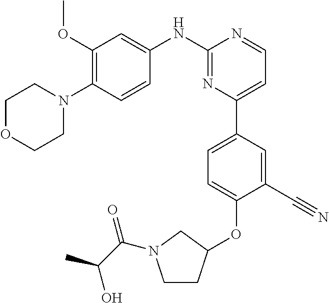 [SMILES: COc1cc(Nc2nccc(-c3ccc(OC4CCN(C(=O)[C@H](C)O)C4)c(C#N)c3)n2)ccc1N1CCOCC1] | 2-({1-[(2S)-2- hydroxypropanoyl] pyrrolidin-3-yl}oxy)-5- (2-{[3-methoxy-4- (morpholin-4- yl)phenyl]amino} pyrimidin-4- yl)benzonitrile | ¹H NMR (DMSO-d₆) Rotamers δ 9.87 (s, 1H), 8.59-8.58 (m, 2H), 8.49-8.46 (m, 1H), 7.87 (br s, 1H), 7.54-7.50 (m, 2H), 7.35-7.25 (m, 2H), 5.41-5.33 (m, 2H), 4.38-4.23 (m, 1H), 3.94 (s, 3H), 3.94-3.77 (m, 5H), 3.76-3.41 (m, 4H), 3.30 (br s, 4H), 2.34-2.10 (m, 2H), 1.23-1.17 (m, 3H). LC-MS [M + H]⁺ 545.2409 |
| 117 | 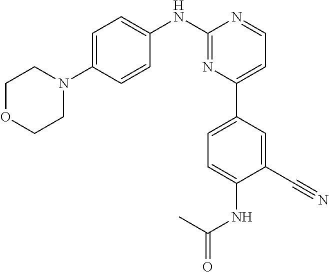 [SMILES: CC(=O)Nc1ccc(-c2ccnc(Nc3ccc(N4CCOCC4)cc3)n2)cc1C#N] | N-[2-Cyano-4-(2- {[4-(morpholin-4- yl)phenyl]amino} pyrimidin-4- yl)phenyl]acetamide | ¹H NMR (DMSO-d₆) Rotamers δ 9.54 (br s, 0.6), 9.41 (br s, 0.4), 8.55-8.37 (m, 3H), 8.26 (d, 0.5H), 8.133-8.10 (m, 0.5H), 7.85-7.42 (m, 3H), 6.99-6.87 (m, 3H), 3.77-3.74 (m, 4H), 3.08 (br s, 4H), 2.16 (s, 3H). LC-MS [M + H]⁺ 415.1856 |
| 118 | 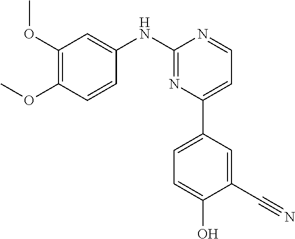 [SMILES: COc1ccc(Nc2nccc(-c3ccc(O)c(C#N)c3)n2)cc1OC] | 5-{3-[(3,4- Dimethoxyphenyl) amino]pyrimidin-4-yl}- 2- hydroxybenzonitrile] | ¹H NMR (DMSO-d₆) δ 9.47 (s, 1H), 8.48 (d, 1H), 8.45 (d, 1H), 8.31- 8.28 (m, 1H), 7.66 (br s, 1H), 7.34 (d, 1H), 7.22-7.12 (m, 2H), 6.90 (d, 1H), 3.80 (s, 3H), 3.73 (s, 3H). LC- MS [M + H]⁺ 349.1311 |
| 119 | 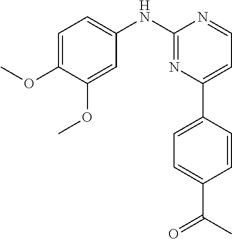 [SMILES: COc1ccc(Nc2nccc(-c3ccc(C(C)=O)cc3)n2)cc1OC] | 1-(4-{2-[(3,4- Dimethoxyphenyl) amino]pyrimidin-4- yl}phenyl)ethanone | ¹H NMR (CDCl₃) δ 8.37 (d, 1H), 8.19-8.16 (m, 2H), 8.10-8.07 (m, 2H), 7.46 (d, 1H), 7.23 (d, 1H), 7.15-7.12 (m, 1H), 6.90 (d, 1H), 3.93 (s, 3H), 3.91 (s, 3H), 2.67 (s, 3H). LC-MS [M + H]⁺ 350.1575 |
| 120 | 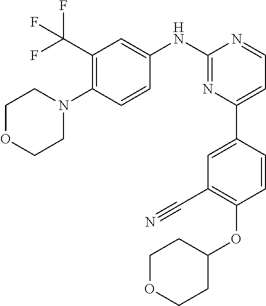 [SMILES: N#Cc1cc(-c2ccnc(Nc3ccc(N4CCOCC4)c(C(F)(F)F)c3)n2)ccc1OC1CCOCC1] | 5-(2-{[4-(morpholin- 4-yl)-3- (trifluoromethyl) phenyl]amino} pyrimidin- 4-yl)-2-(tetrahydro- 2H-pyran-4- yloxy)benzonitrile | ¹H NMR (MeOH-d₄) δ 8.50-8.48 (m, 2H), 8.42 (d, 1H), 8.35-8.34 (br s, 1H), 7.85 (d, 1H), 7.50 (d, 1H), 7.40 (d, 1H), 7.34-7.33 (m, 1H), 4.03-4.00 (m, 2H), 3.82-3.80 (m, 5H), 3.70-3.64 (m, 2H), 2.92-2.90 (m, 4H), 2.15-2.10 (m, 2H), 1.89- 1.80 (m, 2H). LC-MS [M + H]⁺ 526.2125 |
| 121 | 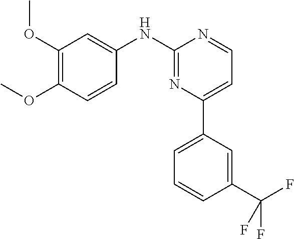 [SMILES: COc1ccc(Nc2nccc(-c3cccc(C(F)(F)F)c3)n2)cc1OC] | N-(3,4- Dimethoxyphenyl)-4- [3- (trifluoromethyl) phenyl]pyrimidin-2- amine | ¹H NMR (CDCl₃) δ 8.50 (d, 1H), 8.37 (s, 1H), 8.22 (d, 1H), 7.75 (d, 1H), 7.64-7.60 (m, 1H), 7.55 (d, 1H), 7.22 (s, 1H), 7.15 (d, 1H), 7.03-7.00 (m, 1H), 6.87 (d, 1H), 3.93 (s, 3H), 3.90 (s, 3H). LC-MS [M + H]⁺ 376.1264 |
| 122 | 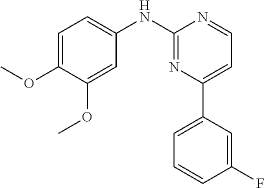 [SMILES: COc1ccc(Nc2nccc(-c3cccc(F)c3)n2)cc1OC] | N-(3,4- Dimethoxyphenyl)-4- (3- fluorophenyl) pyrimidin-2-amine | ¹H NMR (CDCl₃) δ 8.32 (d, 1H), 7.86-7.82 (m, 2H), 7.53-7.47 (m, 2H), 7.30-7.24 (m, 2H), 7.18 (d, 1H), 7.13-7.10 (m, 1H), 6.89 (d, 1H), 3.94 (s, 3H), 3.91 (s, 3H). LC-MS [M + H]⁺ 326.1398 |
| 123 | 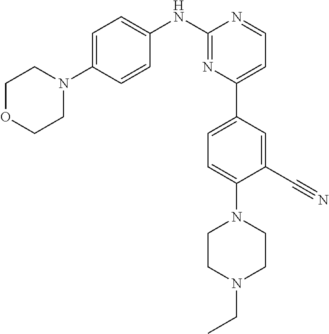 [SMILES: CCN1CCN(c2ccc(-c3ccnc(Nc4ccc(N5CCOCC5)cc4)n3)cc2C#N)CC1] | 2-(4-Ethylpiperazin- 1-yl)-5-(2-{[4- (morpholin-4- yl)phenyl]amino} pyrimidin-4- yl)benzonitrile | ¹H NMR (DMSO-d₆) δ 9.57 (s, 1H), 8.54-8.51 (m, 2H), 8.43-8.40 (m, 1H), 7.68 (d, 1H), 7.42 (d, 1H), 7.42 (s, 1H), 7.03 (d, 2H), 3.87- 3.67 (m, 8H), 3.30-3.12 (m, 10H), 1.27 (t, 3H). LC-MS [M + H]⁺ 470.2682 |
| 124 | 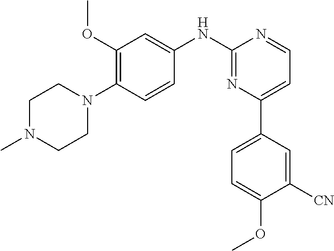 | 2-Methoxy-5-(2-{[3- methoxy-4-(4- methyl-3- oxopiperazin-1- yl)phenyl]amino} pyrimidin-4- yl)benzonitrile] | ¹H NMR (CDCl₃) δ 8.73 (s, 1H), 8.47 (d, 1H), 8.43 (d, 1H), 8.31- 8.28 (m, 1H), 7.70 (bs, 1H), 7.21- 7.11 (m, 3H), 6.87 (d, 1H), 4.03 (s, 3H), 3.95 (s, 3H), 3.73 (s, 2H), 3.48-3.46 (m, 2H), 3.37-3.32 (m, 2H), 3.01 (s, 3H). LC-MS [M + H]⁺ 445.1975 |
| 125 | 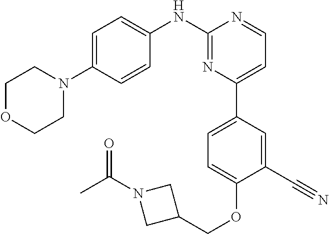 [SMILES: CC(=O)N1CC(COc2ccc(-c3ccnc(Nc4ccc(N5CCOCC5)cc4)n3)cc2C#N)C1] | 2-[(1-acetylazetidin- 3-yl)methoxy]-5-(2- {[4-(morpholin-4- yl)phenyl]amino} pyrimidin-4- yl)benzonitrile | ¹H NMR (DMSO-d₆) δ 9.47 (s, 1H), 8.53-8.45 (m, 3H), 7.65-7.62 (m, 2H), 7.46 (d, 1H), 7.40 (d, 1H), 6.93 (d, 2H), 4.45-4.37 (m, 2H), 4.27 (t, 1H), 4.03-3.94 (m, 2H), 3.76-3.68 (m, 5H), 3.08-3.03 (m, 5H), 1.76 (s, 3H). LC-MS [M + H]⁺ 485.2263 |
| 126 | 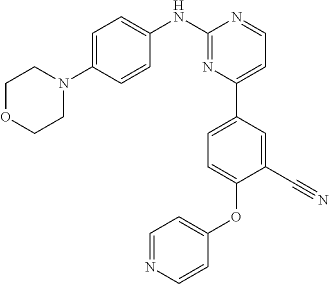 [SMILES: N#Cc1cc(-c2ccnc(Nc3ccc(N4CCOCC4)cc3)n2)ccc1Oc1ccncc1] | 5-(2-{[4-(morpholin- 4- yl)phenyl]amino} pyrimidin-4-yl)-2- (pyridin-4- yloxy)benzonitrile | ¹H NMR (MeOH-d₄) δ 8.69 (d, 1H), 8.41 (d, 1H), 8.20 (d, 1H), 8.17- 8.14 (m, 1H), 7.75-7.762 (m, 3)H, 7.26-7.17 (m, 4H), 7.04 (d, 1H), 3.88-3.86 (m, 4H), 3.28-3.25 (m, 4H). LC-MS [M + H]⁺ 451.1860 |
| 127 | 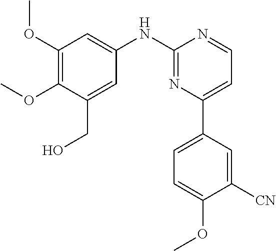 [SMILES: COc1ccc(-c2ccnc(Nc3cc(CO)c(OC)c(OC)c3)n2)cc1C#N] | 5-(2-{[3- (Hydroxymethyl)- 4,5- dimethoxyphenyl] amino}pyrimidin-4-yl)- 2- methoxybenzonitrile | ¹H NMR (CDCl₃) δ 8.47-8.44 (m, 2H), 8.38 (s, 1H), 8.33-8.30 (m, 1H), 7.64 (d, 1H), 7.29 (d, 1H), 7.15-78.09 (m, 2H), 4.71 (d, 2H), 4.14-4.10 (m, 1H), 4.03 (s, 3H), 3.94 (s, 3H), 3.82 (s, 3H). LC-MS [M + H]⁺ 393.2 |
| 128 | 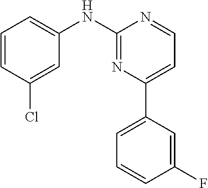 [SMILES: Fc1cccc(-c2ccnc(Nc3cccc(Cl)c3)n2)c1] | N-(3-Chlorophenyl)- 4-(3- fluorophenyl) pyrimidin-2-amine | ¹H NMR (CDCl₃) δ 8.51 (d, 1H), 7.95 (t, 1H), 7.86-7.78 (m, 2H), 7.56 (br s, 1H), 7.52-7.45 (m, 2H), 7.30-7.18 (m, 3H), 7.06-7.03 (m, 1H). LC-MS [M + H]⁺ 300.0661 |
| 129 | 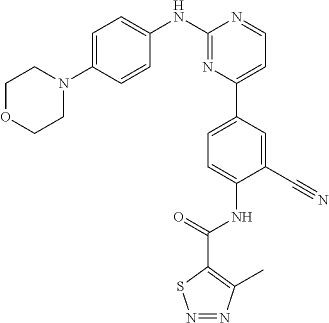 [SMILES: Cc1nnsc1C(=O)Nc1ccc(-c2ccnc(Nc3ccc(N4CCOCC4)cc3)n2)cc1C#N] | N-[2-cyano-4-(2-{[4- (morpholin-4- yl)phenyl]amino} pyrimidin-4-yl)phenyl]- 4-methyl-1,2,3- thiadiazol-5- carboxamide | ¹H NMR (DMSO-d₆) δ 11.30 (s, 1H), 9.55 (s, 1H), 8.65 (d, 1H), 8.57-8.51 (m, 2H), 7.85 (d, 1H), 7.65 (d, 1H), 7.47 (d, 1H), 6.94 (d, 2H), 3.76-3.73 (m, 4H), 3.07-3.04 (m, 4H), 2.90 (s, 3H). LC-MS [M + H]⁺ 499.1545 |
| 130 | 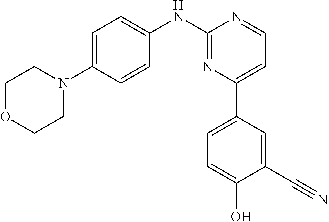 [SMILES: N#Cc1cc(-c2ccnc(Nc3ccc(N4CCOCC4)cc3)n2)ccc1O] | 2-Hydroxy-5-(2-{[4- (morpholin-4- yl)phenyl]amino} pyrimidin-4- yl)benzonitrile | ¹H NMR (DMSO-d₆) δ 9.43 (s, 1H), 8.45 (d, 1H), 8.42 (d, 1H), 8.32- 8.29 (m, 1H), 7.65-7.62 (m, 2H), 7.32 (d, 1H), 7.15 (d, 1H), 6.94- 6.91 (m, 2H), 3.76-3.73 (m, 4H), 3.06-3.03 (m, 4H). LC-MS [M + H]⁺ 374.1662 |
| 131 | 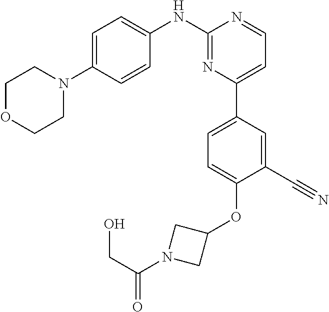 [SMILES: N#Cc1cc(-c2ccnc(Nc3ccc(N4CCOCC4)cc3)n2)ccc1OC1CN(C(=O)CO)C1] | 2-{[1- (hydroxyacetyl) azetidin-3-yl]oxy}-5-(2- {[4-(morpholin-4- yl)phenyl]amino} pyrimidin-4- yl)benzonitrile | ¹H NMR (DMSO-d₆) δ 9.47 (s, 1H), 8.56 (d, 1H), 8.50 (d, 1H), 8.45- 8.42 (m, 1H), 7.63 (d, 2H), 7.40 (d, 1H), 7.18 (d, 1H), 6.93 (d, 2H), 5.33-5.28 (m, 1H), 5.08 (t, 1H), 4.74-4.70 (m, 1H), 4.46-4.42 (m, 1H), 4.29-4.25 (m, 1H), 3.97 (d, 1H), 3.94-3.90 (m, 1H), 3.76-3.73 (m, 4H), 3.06-3.04 (m, 4H). LC- MS [M + H]⁺ 487.2040 |
| 132 | 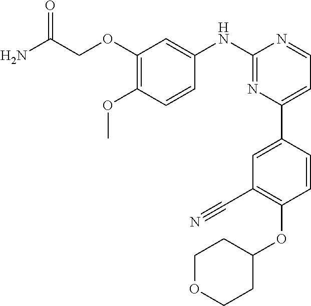 [SMILES: COc1ccc(Nc2nccc(-c3ccc(OC4CCOCC4)c(C#N)c3)n2)cc1OCC(N)=O] | 2-[5-({4-[3-cyano-4- (tetrahydro-2H- pyran-4- yloxy)phenyl] pyrimidin-2-yl}amino)-2- methoxyphenoxy] acetamide | LC-MS [M + H]⁺ 476.1853 |
| 133 | 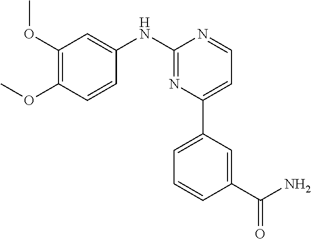 [SMILES: COc1ccc(Nc2nccc(-c3cccc(C(N)=O)c3)n2)cc1OC] | 3-{2-[(3,4- Dimethoxyphenyl) amino]pyrimidin-4- yl}benzamide | ¹H NMR (DMSO-d₆) δ 9.56 (s, 1H), 8.64-8.63 (m, 1H), 8.56 (d, 1H), 8.32-8.29 (m, 1H), 8.14 (br s, 1H), 8.03-8.01 (m, 1H), 7.69 (br s, 1H), 7.65-7.61 (m, 1H), 7.52 (br s, 1H), 7.43 (d, 1H), 7.27-7.23 (m, 1H), 6.91 (d, 1H), 3.78 (s, 3H), 3.73 (s, 3H). LC-MS [M + H]⁺ 373.1236 |
| 134 | 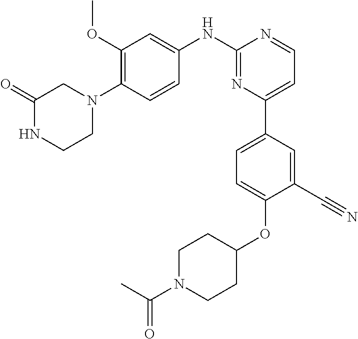 [SMILES: COc1cc(Nc2nccc(-c3ccc(OC4CCN(C(C)=O)CC4)c(C#N)c3)n2)ccc1N1CCNC(=O)C1] | 2-[(1- Acetylpiperidin-4- yl)oxy]-5-(2-{[3- methoxy-4-(3- oxopiperazin-1- yl)phenyl]amino} pyrimidin-4- yl)benzonitrile | ¹H NMR (CDCl₃) δ 8.47 (d, 1H), 8.37 (d, 1H), 8.25-8.22 (m, 1H), 7.54-7.53 (m, 1H), 7.11-7.06 (m, 3H), 6.92 (d, 1H), 4.85-4.81 (m, 1H), 3.96 (s, 3H), 3.94-3.91 (m, 1H), 3.81 (s, 2H), 3.79-3.73 (m, 1H), 3.65-3.48 (m, 4H), 3.34 (t, 2H), 2.14 (s, 3H), 2.02-1.93 (m, 4H). LC-MS [M + H]⁺ 542.2585 |
| 135 | 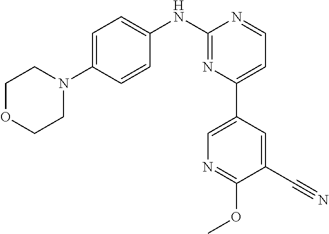 [SMILES: COc1ncc(-c2ccnc(Nc3ccc(N4CCOCC4)cc3)n2)cc1C#N] | 2-methoxy-5-(2-{[4- (morpholin-4- yl)phenyl]amino} pyrimidin-4-yl)pyridine- 3-carbonitrile | ¹H NMR (DMSO-d₆) δ 9.53 (s, 1H), 9.20 (d, 1H), 8.92 (d, 1H), 8.53 (d, 1H), 7.62 (d, 2H), 7.41 (d, 1H), 6.94 (d, 1H), 4.09 (s, 3H), 3.74 (m, 4H), 3.05 (m, 4H); LC-MS [M + H]⁺ 389.1723 |
| 136 | 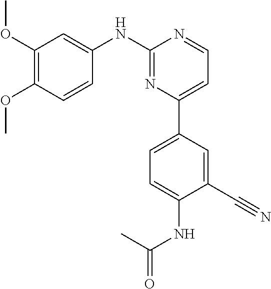 [SMILES: COc1ccc(Nc2nccc(-c3ccc(NC(C)=O)c(C#N)c3)n2)cc1OC] | N-(2-Cyano-4-{2- [(3,4- dimethoxyphenyl) amino]pyrimidin-4- yl}phenyl)acetamide | ¹H NMR (CDCl₃) δ 8.61 (d, 1H), 8.46 (d, 1H), 8.38 (d, 1H), 8.26- 8.23 (m, 1H), 7.71 (br s, 1H), 7.45 (d, 1H), 7.09-7.04 (m, 2H), 6.89 (d, 1H), 3.95 (s, 3H), 3.90 (s, 3H), 2.32 (s, 3H). LC-MS [M + H]⁺ 390.1556 |
| 137 | 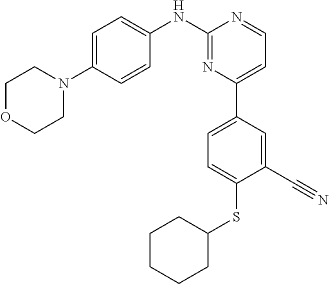 [SMILES: N#Cc1cc(-c2ccnc(Nc3ccc(N4CCOCC4)cc3)n2)ccc1SC1CCCCC1] | 2- (cyclohexylsulfanyl)- 5-(2-{[4-(morpholin- 4- yl)phenyl]amino} pyrimidin-4- yl)benzonitrile | ¹H NMR (DMSO-d₆) δ 9.52 (s, 1H), 8.56-8.52 (m, 2H), 8.41-8.38 (m, 1H), 7.80 (d, 1H), 7.65-7.62 (m, 2H), 7.43 (d, 1H), 6.93 (d, 2H), 3.76-3.73 (m, 4H), 3.69-3.62 (m, 1H), 3.35 (s, 1H), 3.06-3.03 (m, 4H), 2.03-1.97 (m, 2H), 1.76-1.72 (m, 2H), 1.64-1.59 (m, 1H), 1.48- 1.37 (m, 4H), 1.34-1.26 (m, 1H). LC-MS [M + H]⁺ 472.2051 |
| 138 | 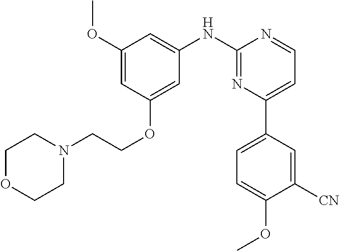 [SMILES: COc1cc(Nc2nccc(-c3ccc(OC)c(C#N)c3)n2)cc(OCCN2CCOCC2)c1] | 2-Methoxy-5-[2-({3- methoxy-5-[2- (morpholin-4- yl)ethoxy]phenyl} amino)pyrimidin-4- yl]benzonitrile | ¹H NMR (CDCl₃) δ 8.48 (d, 1H), 8.37 (d, 1H), 8.27-8.24 (m, 1H), 7.20 (bs, 1H), 7.09-7.07 (m, 2H), 6.99-6.91 (m, 2H), 6.22 (s, 1H), 4.16-4.13 (m, 2H), 4.02 (s, 3H), 3.84 (s, 3H), 3.75-3.73 (m, 4H), 2.85-2.82 (m, 2H), 2.60-2.58 (m, 4H). LC-MS [M + H]⁺ 462.2134 |
| 139 | 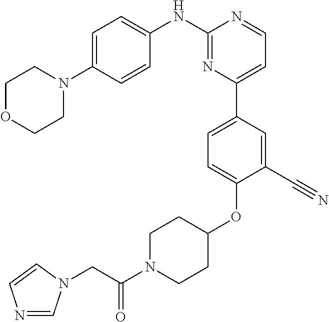 [SMILES: N#Cc1cc(-c2ccnc(Nc3ccc(N4CCOCC4)cc3)n2)ccc1OC1CCN(C(=O)Cn2ccnc2)CC1] | 2-{[1-(1H-imidazol- 1-ylacetyl)piperidin- 4-yl]oxy}-5-(2-{[4- (morpholin-4- yl)phenyl]amino} pyrimidin-4- yl)benzonitrile | ¹H NMR (CDCl₃) δ 8.45 (d, 1H), 8.32 (d, 1H), 8.26-8.22 (m, 1H), 7.70 (s, 1H), 7.55-7.53 (m, 2H), 7.15-7.14 (m, 1H), 7.10-6.95 (m, 6H), 4.96-4.77 (m, 3H), 4.16-4.08 (m, 1H), 3.90-3.87 (m, 4H), 3.82- 3.74 (m, 1H), 3.58-3.50 (m, 2H), 3.16-3.13 (m, 4H), 1.52-1.41 (m, 1H), 1.25-1.15 (m, 1H). LC-MS [M + H]⁺ 565.2718 |
| 140 | 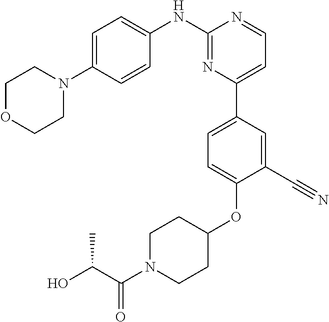 [SMILES: C[C@@H](O)C(=O)N1CCC(Oc2ccc(-c3ccnc(Nc4ccc(N5CCOCC5)cc4)n3)cc2C#N)CC1] | 2-({1-[(2R)-2- hydroxypropanoyl] piperidin-4-yl}oxy)-5- (2-{[4-(morpholin-4- yl)phenyl]amino} pyrimidin-4- yl)benzonitrile | ¹H NMR (DMSO-d₆) δ 9.65 (s, 1H), 8.54-8.51 (m, 2H), 8.47-8.44 (m, 1H), 7.72 (d, 2H), 7.56 (d, 1H), 7.44 (d, 1H), 7.11 (br s, 2H), 5.01 (br s, 1H), 4.49-4.44 (m, 1H), 3.82- 3.79 (m, 4H), 3.74-3.68 (m, 2H), 3.58-3.36 (m, 2H), 3.20 (br s, 4H), 2.06-1.92 (m, 2H), 1.80-1.62 (m, 2H), 1.20 (d, 3H). LC-MS [M + H]⁺ 529.2754 |
| 141 | 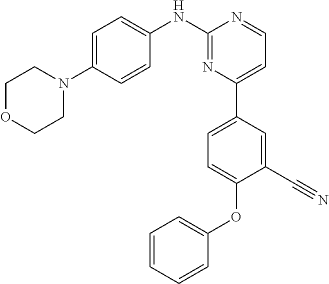 [SMILES: N#Cc1cc(-c2ccnc(Nc3ccc(N4CCOCC4)cc3)n2)ccc1Oc1ccccc1] | 5-(2-{[4-(morpholin- 4- yl)phenyl]amino} pyrimidin-4-yl)-2- phenoxybenzonitrile | ¹H NMR (CDCl₃) δ 8.21 (d, 1H), 7.88-7.84 (m, 2H), 7.46-7.41 (m, 2H), 7.35-7.32 (m, 5H), 7.12 (d, 2H), 7.05-7.01 (m, 2H), 3.96-3.93 (m, 4H), 3.32 (br s, 4H). LC-MS [M + H]⁺ 450.1865 |
| 142 | 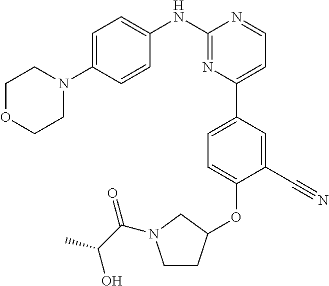 [SMILES: C[C@@H](O)C(=O)N1CCC(Oc2ccc(-c3ccnc(Nc4ccc(N5CCOCC5)cc4)n3)cc2C#N)C1] | 2-({1-[(2R)-2- hydroxypropanoyl] pyrrolidin-3-yl}oxy)-5- (2-{[4-(morpholin-4- yl)phenyl]amino} pyrimidin-4- yl)benzonitrile | LC-MS [M + H]⁺ 515.2388 |
| 143 | 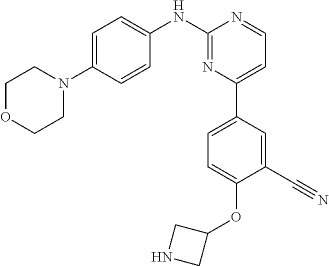 [SMILES: N#Cc1cc(-c2ccnc(Nc3ccc(N4CCOCC4)cc3)n2)ccc1OC1CNC1] | 2-(azetidin-3-yloxy)- 5-(2-{[4-(morpholin- 4- yl)phenyl]amino} pyrimidin-4- yl)benzonitrile | ¹H NMR (DMSO-d₆) δ 9.46 (s, 1H), 8.53 (d, 1H), 8.48 (d, 1H), 8.42- 8.39 (m, 1H), 7.65-7.62 (m, 2H), 7.37 (d, 1H), 7.13 (d, 1H), 6.92 (d, 2H), 5.27-5.20 (m, 1H), 3.88-3.83 (m, 2H), 3.76-3.73 (m, 4H), 3.60- 3.55 (m, 2H), 3.34 (br s, 1H), 3.06- 3.03 (m, 4H). LC-MS [M + H]⁺ 429.1945 |
| 144 | 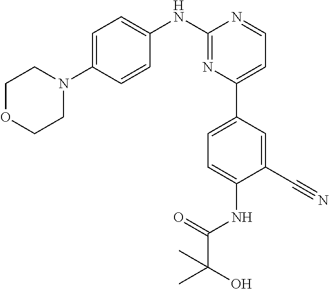 [SMILES: CC(C)(O)C(=O)Nc1ccc(-c2ccnc(Nc3ccc(N4CCOCC4)cc3)n2)cc1C#N] | N-[2-Cyano-4-(2- {[4-(morpholin-4- yl)phenyl]amino} pyrimidin-4-yl)phenyl]- 2-hydroxy-2- methylpropanamide | ¹H NMR (DMSO-d₆) δ 9.62 (s, 1H), 8.52 (d, 1H), 8.31 (d, 1H), 8.29 (d, 1H), 8.11-8.08 (m, 1H), 7.55 (d, 1H), 7.48 (d, 2H), 6.85-6.80 (m, 4H), 3.72-3.70 (m, 4H), 3.04-3.01 (m, 4H), 1.68 (s, 6H). LC-MS [M + H]⁺ 459.2112 |
| 145 | 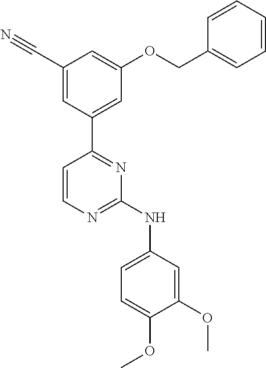 [SMILES: COc1ccc(Nc2nccc(-c3cc(C#N)cc(OCc4ccccc4)c3)n2)cc1OC] | 3-(Benzyloxy)-5-{2- [(3,4- dimethoxyphenyl) amino]pyrimidin-4- yl}benzonitrile | ¹H NMR (CDCl₃) δ 8.49 (d, 1H), 7.95-7.94 (m, 2H), 7.53 (s, 1H), 7.43-7.37 (m, 5H), 7.31-7.30 (m, 1H), 7.21 (1H), 7.07 (d, 1H), 7.02- 6.99 (m, 1H), 6.87 (d, 1H), 5.15 (s, 2H), 3.93 (s, 3H), 3.88 (s, 3H). LC-MS [M + H]⁺ 439.1824 |
| 146 | 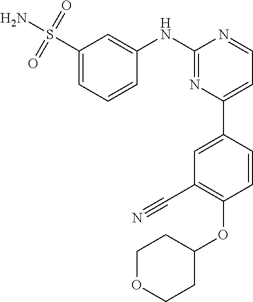 [SMILES: N#Cc1cc(-c2ccnc(Nc3cccc(S(N)(=O)=O)c3)n2)ccc1OC1CCOCC1] | 3-({4-[3-cyano-4- (tetrahydro-2H- pyran-4- yloxy)phenyl] pyrimidin-2- yl}amino) benzenesulfonamide | ¹H NMR (MeOH-d₄) δ 8.60 (d, 2H), 8.50 (d, 1H), 8.46 (d, 1H), 8.40 (d, 1H), 7.59 (d, 2H), 7.40 (d, 2H), 4.10 (s, 1H), 4.02-3.97 (m, 2H), 3.70-3.64 (m, 2H), 2.15-2.10 (m, 2H), 1.90-1.80 (m, 2H). LC- MS [M + Na]⁺ 474.1210 |
| 147 | 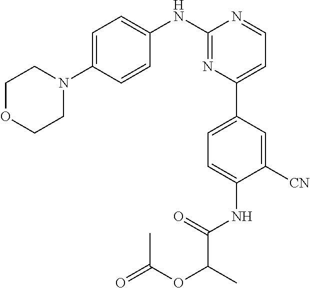 [SMILES: CC(=O)OC(C)C(=O)Nc1ccc(-c2ccnc(Nc3ccc(N4CCOCC4)cc3)n2)cc1C#N] | 1-{[2-cyano-4-(2- {[4-(morpholin-4- yl)phenyl]amino} pyrimidin-4- yl)phenyl]amino}-1- oxopropan-2-yl acetate | ¹H NMR (CDCl₃) δ 8.80 (s, 1H), 8.67 (d, 1H), 8.43 (d, 1H), 8.36 (d, 1H), 8.28 (dd, 2H), 7.62 (bs, 1H), 7.57 (m, 2H), 7.07 (d, 1H), 6.98 (m, 2H), 5.1-5.45 (m, 1H), 3.88 (m, 4H), 3.16 (m, 4H), 2.29 (s, 3H), 1.6 (m, 3H). LC-MS [M + H]⁺ 487.2066 |
| 148 | 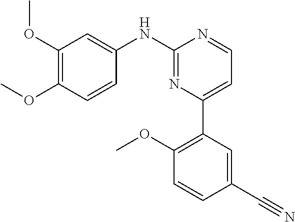 [SMILES: COc1ccc(Nc2nccc(-c3cc(C#N)ccc3OC)n2)cc1OC] | 3-{2-[(3,4- Dimethoxyphenyl) amino]pyrimidin-4-yl}- 4- methoxybenzonitrile | ¹H NMR (CDCl₃) δ 8.44 (d, m1H), 8.37 (d, 1H), 7.74-7.71 (m, 1H), 7.51 (d, 1H), 7.32 (d, 1H), 7.10 (s, 1H), 7.08 (d, 1H), 7.02-6.99 (m, 1H), 6.87 (d, 1H), 3.98 (s, 3H), 3.95 (s, 3H), 3.89 (s, 3H). LC-MS [M + H]⁺ 363.1509 |
| 149 | 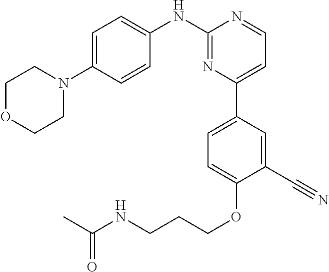 [SMILES: CC(=O)NCCCOc1ccc(-c2ccnc(Nc3ccc(N4CCOCC4)cc3)n2)cc1C#N] | N-{3-[2-cyano-4-(2- {[4-(morpholin-4- yl)phenyl]amino} pyrimidin-4- yl)phenoxy]propyl} acetamide | ¹H NMR (DMSO-d₆) δ 9.47 (s, 1H), 8.52-8.44 (m, 3H), 7.99-7.96 (m, 1H), 7.65-7.61 (m, 2H), 7.41 (d, 1H), 7.39 (d, 1H), 6.93 (d, 2H), 4.25 (t, 2H), 3.76-3.73 (m, 4H), 3.26-3.21 (m, 2H), 3.06-3.03 (m, 4H), 1.95-1.88 (m, 2H), 1.81 (s, 3H). LC-MS [M + H]⁺ 473.2351 |
| 150 | 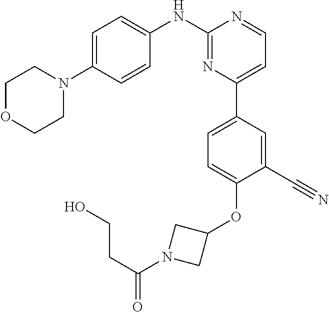 [SMILES: N#Cc1cc(-c2ccnc(Nc3ccc(N4CCOCC4)cc3)n2)ccc1OC1CN(C(=O)CCO)C1] | 2-{[1-(3- hydroxypropanoyl) azetidin-3-yl]oxy}-5- (2-{[4-(morpholin-4- yl)phenyl]amino} pyrimidin-4- yl)benzonitrile | ¹H NMR (DMSO-d₆) δ 9.55 (s, 1H), 8.57 (d, 1H), 8.51 (d, 1H), 8.46- 8.43 (m, 1H), 7.66 (d, 2H), 7.42 (d, 1H), 7.20 (d, 1H), 7.00 (d, 2), 5.32-5.26 (m, 1H), 4.69-4.65 (m, 1H), 4.41-4.37 (m, 1H), 4.25-4.22 (m, 1H), 3.89-3.85 (m, 1H), 3.77 (br s, 4H), 3.62 (t, 2H), 3.10 (br s, 4H), 2.26 (t, 2H). LC-MS [M + H]⁺ 501.2113 |
| 151 | 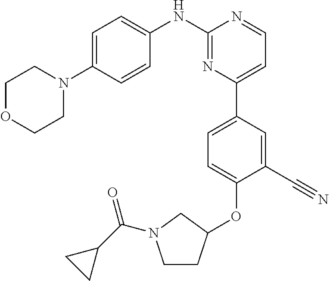 [SMILES: N#Cc1cc(-c2ccnc(Nc3ccc(N4CCOCC4)cc3)n2)ccc1OC1CCN(C(=O)C2CC2)C1] | 2-{[1- (cyclopropylcarbonyl) pyrrolidin-3- yl]oxy}-5-(2-{[4- (morpholin-4- yl)phenyl]amino} pyrimidin-4- yl)benzonitrile | LC-MS [M + H]⁺ 511.2440 |
| 152 | 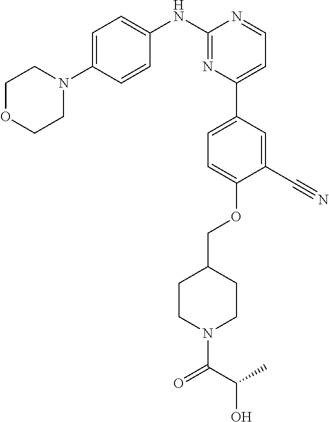 [SMILES: C[C@H](O)C(=O)N1CCC(COc2ccc(-c3ccnc(Nc4ccc(N5CCOCC5)cc4)n3)cc2C#N)CC1] | 2-({1-[(2S)-2- hydroxypropanoyl] piperidin-4- yl}methoxy)-5-(2- {[4-(morpholin-4- yl)phenyl]amino} pyrimidin-4- yl)benzonitrile | ¹H NMR (DMSO-d₆) δ 9.46 (s, 1H), 8.51-8.44 (m, 3H), 7.63 (d, 2H), 7.45-7.38 (m, 2H), 6.92 (d, 2H), 4.83-4.80 (m, 1H), 4.47-4.38 (m, 2H), 4.05-4.01 (m, 1H), 3.76-3.73 (m, 4H), 3.18-3.11 (m, 2H), 3.06- 3.03 (m, 4H), 2.76-2.70 (m, 1H)2.13 (br s, 1H), 1.89-1.80 (m, 2H), 1.25 (d, 3H), 1.18 (t, 2H). LC- MS [M + H]⁺ 543.2587 |
| 153 | 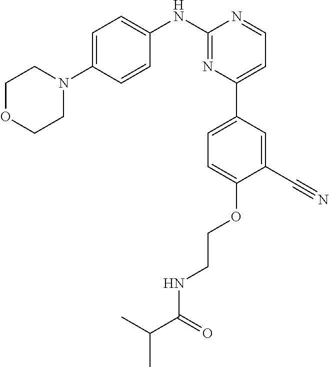 [SMILES: CC(C)C(=O)NCCOc1ccc(-c2ccnc(Nc3ccc(N4CCOCC4)cc3)n2)cc1C#N] | N-{2-[2-cyano-4-(2- {[4-(morpholin-4- yl)phenyl]amino} pyrimidin-4- yl)phenoxy]ethyl}-2- methylpropanamide | ¹H NMR (DMSO-d₆) δ 9.50 (s, 1H), 8.52-8.43 (m, 3H), 8.05-8.02 (m, 1H), 7.65 (d, 2H), 7.47 (d, 1H), 7.41 (d, 1H), 6.96 (d, 2H), 4.28 (t, 2H), 3.77-3.74 (m, 4H), 3.49-3.45 (m, 2H), 3.09-3.06 (m, 4H), 2.42- 2.l35 (m, 1H), 1.01 (d, 6H). LC-MS [M + H]⁺ 487.2304 |
| 154 | 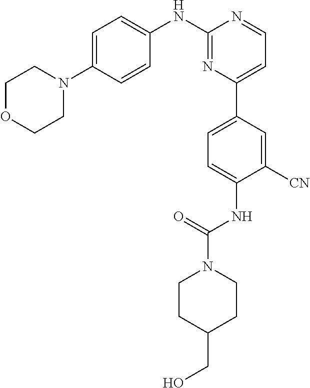 [SMILES: N#Cc1cc(-c2ccnc(Nc3ccc(N4CCOCC4)cc3)n2)ccc1NC(=O)N1CCC(CO)CC1] | N-[2-cyano-4-(2-{[4- (morpholin-4- yl)phenyl]amino} pyrimidin-4-yl)phenyl]- 4- (hydroxymethyl) piperidine-1- carboxamide | ¹H NMR (CDCl₃) δ 8.44 (dd, 2H), 8.29 (d, 1H), 8.21 (dd, 1H),) 7.55 (dd, 2H), 7.21 (s, 1H), 7.07 ( s, 1H), 7.04 ( d, 1H), 76.96 (dd, 2H), 4.19 (d, 2H), 3.89 (m, 4H), 3.56 (m, 2H), 3.15 (m, 4H), 3.01 (m, 2H), 1.90 (m, 2H), 1.8 (m, 1H), 1.33 (m, 2H). LC-MS [M + H]⁺ 514.2538 |
| 155 | 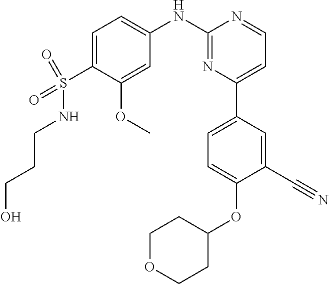 [SMILES: COc1cc(Nc2nccc(-c3ccc(OC4CCOCC4)c(C#N)c3)n2)ccc1S(=O)(=O)NCCCO] | 4-({4-[3-cyano-4- (tetrahydro-2H- pyran-4- yloxy)phenyl] pyrimidin-2-yl} amino)-N- (3-hydroxypropyl)-2- methoxybenzene- sulfonamide | ¹H NMR (DMSO-d₆) δ 10.14 (s, 1H), 8.63 (d, 1H), 8.59 (d, 1H), 8.47 (d, 1H); 7.98 (s, 1H); 7.64-7.54 (m, 3H); 7.40 (d, 1H); 6.93 (t, 1H); 4.98-4.94 (m, 1H); 4.41 (t, 1H); 3.95 (s, 3H); 3.93-3.86 (m, 3H); 3.59-3.54 (m, 2H); 2.81-2.76 (m, 2H); 2.20-2.00 (m, 3H 1.75-1.67 (m, 2H); 1.55-1.49 (m, 2H). LC- MS [M + H]⁺ 540.1822[M + Na]⁺562.1650 |
| 156 | 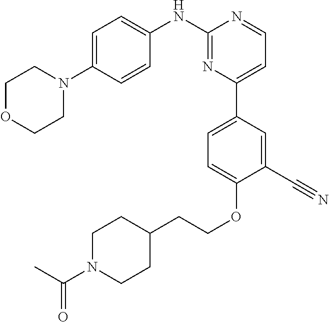 [SMILES: CC(=O)N1CCC(CCOc2ccc(-c3ccnc(Nc4ccc(N5CCOCC5)cc4)n3)cc2C#N)CC1] | 2-[2-(1- acetylpiperidin-4- yl)ethoxy]-5-(2-{[4- (morpholin-4- yl)phenyl}amino} pyrimidin-4- yl)benzonitrile | ¹H NMR (DMSO-d₆) δ 9.50 (br s, 1H), 8.51-8.43 (m, 3H), 7.65 (d, 2H), 7.45 (d, 1H), 7.40 (d, 1H), 7.00-6.94 (m, 1H), 4.39-4.34 (m, 1H), 4.30-4.27 (m, 2H), 3.83-3.74 (m, 5H), 3.11-2.96 (m, 5H), 2.51- 2.49 (m, 1H), 1.99 (s, 3H), 1.79- 1.72 (m, 5H), 1.22-1.02 (m, 2H). LC-MS [M + H]⁺ 527.2596 |
| 157 | 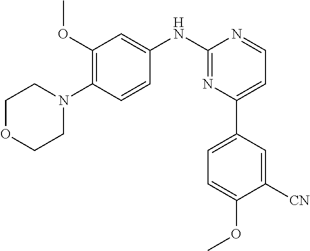 [SMILES: COc1ccc(-c2ccnc(Nc3ccc(N4CCOCC4)c(OC)c3)n2)cc1C#N] | 2-Methoxy-5-(2-{[3- methoxy-4- (morpholin-4- yl)phenyl]amino} pyrimidin-4- yl)benzonitrile | ¹H NMR (DMSO-d₆) δ 9.55 (s, 1H), 8.55-8.47 (m, 3H), 7.64 (bs, 1H), 7.45-7.37 (m, 2H), 7.25 (d, 1H) 6.85 (d, 1H), 4.01 (s, 3H), 3.83 (s, 3H), 3.78-3.69 (m, 4H), 3.00-2.92 (m, 4H). LC-MS [M + H]⁺ 418.1879 |
| 158 | 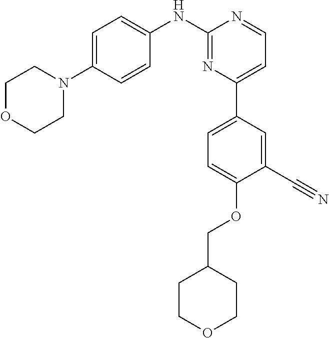 [SMILES: N#Cc1cc(-c2ccnc(Nc3ccc(N4CCOCC4)cc3)n2)ccc1OCC1CCOCC1] | 5-(2-{[4-(morpholin- 4- yl)phenyl]amino} pyrimidin-4-yl)-2- (tetrahydro-2H- pyran-4- ylmethoxy) benzonitrile | ¹H NMR (DMSO-d₆) δ 9.46 (s, 1H), 8.51-8.44 (m, 3H), 7.63 (d, 2H), 7.44 (d, 1H), 7.29 (d, 1H), 6.92 (d, 2H), 4.11 (d, 2H), 3.92-3.88 (m, 2H), 3.76-3.73 (m, 4H), 3.39-3.33 (m, 2H), 3.06-3.03 (m, 4H), 2.14- 2.04 (m, 1H), 1.74-1.67 (m, 2H), 1.45-1.34 (m, 2H). LC-MS [M + H]⁺ 472.2305 |
| 159 | 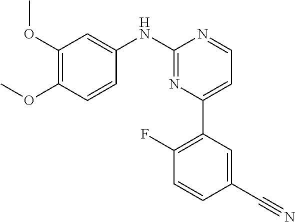 [SMILES: COc1ccc(Nc2nccc(-c3cc(C#N)ccc3F)n2)cc1OC] | 3-{2-[(3,4- Dimethoxyphenyl) amino]pyrimidin-4-yl}- 4-fluorobenzonitrile | ¹H NMR (CDCl₃) δ 8.56-8.54 (m, 1H), 8.51 (d, 1H), 7.77-7.73 (m, 1H), 7.46 (d, 1H), 7.33-7.28 (m, 1H), 7.25-7.22 (m, 1H), 7.17 (s, 1H), 7.04-7.01 (m, 1H), 6.88 (d, 1H), 3.96 (s, 3H), 3.90 (s, 3H). LC-MS [M + H]⁺ 351.1315 |
| 160 | 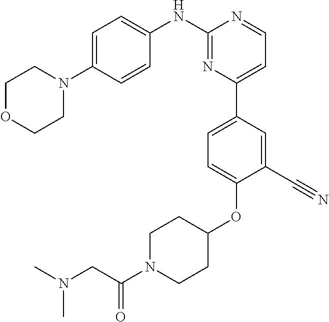 [SMILES: CN(C)CC(=O)N1CCC(Oc2ccc(-c3ccnc(Nc4ccc(N5CCOCC5)cc4)n3)cc2C#N)CC1] | 2-{[1-(N,N- dimethylglycyl)piperidin- 4-yl]oxy}-5-(2- {[4-(morpholin-4- yl)phenyl]amino} pyrimidin-4- yl)benzonitrile | ¹H NMR (CDCl₃) δ 8.44 (d, 1H), 8.31 (d, 1H), 8.25-8.22 (m, 1H), 7.56-7.53 (m, 2H), 7.11-6.94 (m, 3H), 4.84-4.80 (m, 1H), 3.90-3.80 (m, 6H), 3.72-3.59 (m, 3H), 3.24- 3.12 (m, 6H), 2.32 (s, 6H), 2.05- 1.92 (m, 4H). LC-MS [M + H]⁺ 542.2961 |
| 161 | 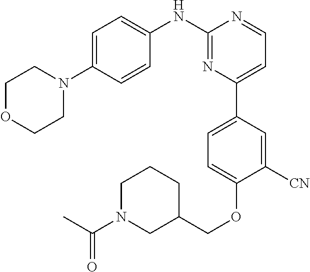 [SMILES: CC(=O)N1CCCC(COc2ccc(-c3ccnc(Nc4ccc(N5CCOCC5)cc4)n3)cc2C#N)C1] | 2-[(1- acetylpiperidin-3- yl)methoxy{-5-(2- {[4-(morpholin-4- yl)phenyl]amino} pyrimidin-4- yl)benzonitrile | ¹H NMR (DMSO-d₆) δ 9.47 (s, 1H), 8.53-8.43 (m, 3H), 7.64 (d, 2H), 7.47-7.38 (m, 2H), 6.93 (d, 2H), 4.37-4.02 (m, 3H), 3.91-3.69 (m, 5H), 3.35 (s, 2H), 3.12-3.03 (m, 5H), 2.85-2.62 (m, 1H), 2.07-1.86 (m, 2H), 2.00 (s, 3H), 1.72-1.35 (m, 3H). LC-MS [M + H]⁺ 513.2658 |
| 162 | 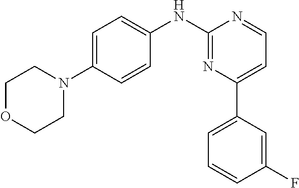 [SMILES: Fc1cccc(-c2ccnc(Nc3ccc(N4CCOCC4)cc3)n2)c1] | 4-(3-Fluorophenyl)- N-[4-(morpholin-4- yl)phenyl]pyrimidin- 2-amine | ¹H NMR (CDCl₃) δ 8.45 (d, 1H), 7.83-7.77 (m, 2H), 7.59-7.55 (m, 2H), 7.47-7.43 (m, 1H), 7.21-7.16 (m, 1H), 7.11 (s, 1H), 7.08 (d, 1H), 6.98-6.94 (d, 2H), 3.90-3.87 (m, 4H), 3.16-3.13 (m, 4H). LC-MS [M + H]⁺ 351.1615 |
| 163 | 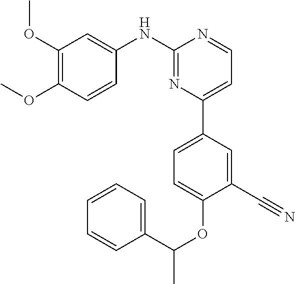 [SMILES: COc1ccc(Nc2nccc(-c3ccc(OC(C)c4ccccc4)c(C#N)c3)n2)cc1OC] | 5-{3-[(3,4- Dimethoxyphenyl) amino]pyrimidin-4-yl}- 2-(1- phenylethoxy) benzonitrile | ¹H NMR (CDCl₃) δ 8.34 (d, 1H), 8.32 (d, 1H), 8.07-8.04 (m, 1H), 7.42-7.30 (m, 5H), 7.06-7.03 (m, 1H), 7.01 (d, 1H), 6.92-6.86 (m, 2H), 5.52-5.47 (m, 1H), 3.91 (s, 3H), 3.90 (s, 3H), 1.77 (d, 3H). LC-MS [M + H]⁺ 453.1944 |
| 164 | 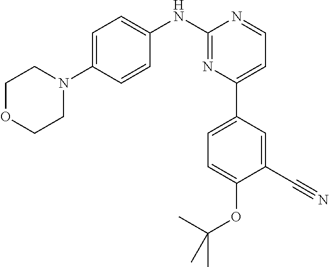 [SMILES: CC(C)(C)Oc1ccc(-c2ccnc(Nc3ccc(N4CCOCC4)cc3)n2)cc1C#N] | 2-tert-Butyoxy-5-(2- {[4-(morpholin-4- yl)phenyl]amino} pyrimidin-4- yl)benzonitrile | ¹H NMR (CDCl₃) δ 8.44 (d, 1H), 8.28 (d, 1H), 8.18-8.15 (m, 1H), 7.56-7.53 (m, 2H), 7.27-7.23 (m, 2H), 7.03 (d, 1H), 6.98-6.95 (m, 2H), 3.90-3.87 (m, 4H), 3.16-3.13 (m, 4H), 1.54 (s, 9H). LC-MS [M + H]⁺ 430.2314 |
| 165 | 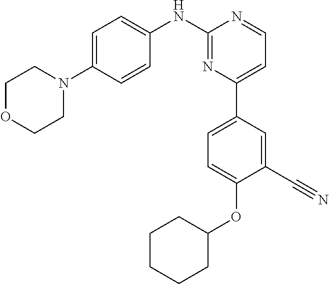 [SMILES: N#Cc1cc(-c2ccnc(Nc3ccc(N4CCOCC4)cc3)n2)ccc1OC1CCCCC1] | 2-(Cyclohexyloxy)- 5-(2-{[4-(morpholin- 4- yl)phenyl]amino} pyrimidin-4- yl)benzonitrile | ¹H NMR (CDCl₃) δ 8.43 (d, 1H), 8.29 (d, 1H), 8.21-8.18 (m, 1H), 7.56-7.53 (m, 2H), 7.10-6.95 (m, 5H), 4.53-4.47 (m, 1H), 3.90-3.87 (m, 4H), 3.16-3.13 (m, 4H), 1.99- 1.40 (m, 10H). LC-MS [M + H]⁺ 456.2357 |
| 166 | 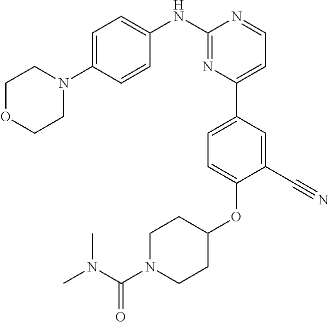 [SMILES: CN(C)C(=O)N1CCC(Oc2ccc(-c3ccnc(Nc4ccc(N5CCOCC5)cc4)n3)cc2C#N)CC1] | 4-[2-Cyano-4-(2-{[4- (morpholin-4- yl)phenyl]amino} pyrimidin-4- yl)phenoxy]-N,N- dimethylpiperidine- 1-carboxamide | ¹H NMR (CDCl₃) δ 8.43 (d, 1H), 8.30 (d, 1H), 8.24-8.20 (m, 1H), 7.56-7.53 (m, 2H), 7.15 (s, 1H), 7.08 (d, 1H), 7.01 (d, 1H), 6.97- 6.95 (m, 2H), 4.76-4.71 (m, 1H), 3.90-3.87 (m, 4H), 3.58-3.51 (m, 2H), 3.29-3.24 (m, 2H), 3.16-3.13 (m, 4H), 2.85 (s, 6H), 2.09-1.88 (m, 4H). LC-MS [M + H]⁺ 528.2776 |
| 167 | 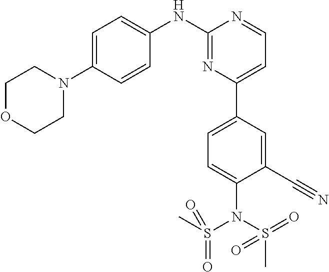 [SMILES: CS(=O)(=O)N(c1ccc(-c2ccnc(Nc3ccc(N4CCOCC4)cc3)n2)cc1C#N)S(C)(=O)=O] | N-[2-Cyano-4-(2- {[4-(morpholin-4- yl)phenyl]amino} pyrimidin-4-yl)phenyl]- N- (methylsulfonyl) methanesulfonamide | ¹H NMR (CDCl₃) δ 8.41 (d, 1H), 8.25 (d, 1H), 8.13-8.10 (m, 1H), 7.73 (d, 1H), 7.57-7.54 (m, 2H), 7.08 (s, 1H), 7.01 (d, 1H), 6.97- 6.95 (m, 2H), 3.90-3.87 (m, 4H), 3.16-3.13 (m, 4H), 1.25 (s, 6H). LC-MS [M + H]⁺ 529.1201 |
| 168 | 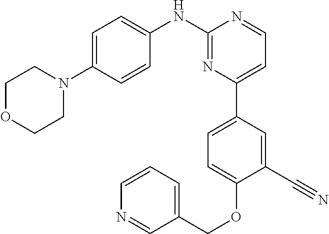 [SMILES: N#Cc1cc(-c2ccnc(Nc3ccc(N4CCOCC4)cc3)n2)ccc1OCc1cccnc1] | 5-(2-{[4-(Morpholin- 4- yl)phenyl]amino} pyrimidin-4-yl)-2- (pyridin-3- ylmethoxy) benzonitrile | ¹H NMR (CDCl₃) δ 8.72 (s, 1H), 8.66-8.63 (m, 1H), 8.44 (d, 1H), 8.34 (d, 1H), 8.25-8.22 (m, 1H), 7.91-7.88 (m, 1H), 7.55-7.52 (m, 2H), 7.41-7.37 (m, 1H), 7.13 (d, 1H), 7.08 (s, 1H), 7.02 (d, 1H), 6.98-6.95 (m, 2H), 5.31 (s, 2H), 3.90-3.87 (m, 4H), 3.16-3.13 (m, 4H). LC-MS [M + H]⁺ 465.2011 |
| 169 | 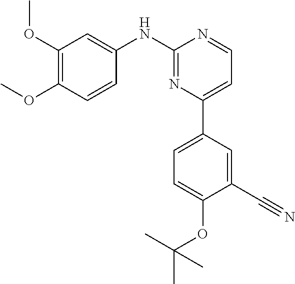 [SMILES: COc1ccc(Nc2nccc(-c3ccc(OC(C)(C)C)c(C#N)c3)n2)cc1OC] | 2-tert-Butoxy-5-{2- [(3,4- dimethoxyphenyl) amino]pyrimidin-4- yl}benzonitrile | ¹H NMR (CDCl₃) δ 8.46 (d, 1H), 8.33 (d, 1H), 8.18-8.15 (m, 1H), 7.46 (d, 1H), 7.24 (d, 1H), 7.14 (br s, 1H), 7.06-7.03 (m, 2H), 6.88 (d, 1H), 3.95 (s, 3H), 3.90 (s, 3H), 1.54 (s, 9H). LC-MS [M + H]⁺ 405.1913 |
| 170 | 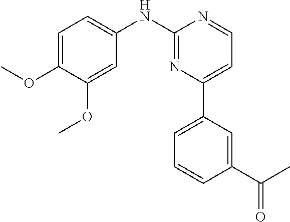 [SMILES: COc1ccc(Nc2nccc(-c3cccc(C(C)=O)c3)n2)cc1OC] | 1-(3-{2-[(3,4- Dimethoxyphenyl) amino]pyrimidin-4- yl}phenyl)ethanone | ¹H NMR (CDCl₃) δ 8.67-8.66 (m, 1H), 8.34-8.29 (m, 2H), 8.16-8.13 (m, 1H), 7.64 (t, 1H), 7.42 (d, 1H), 7.29 (d, 1H), 7.20-7.16 (m, 1H), 6.90 (d, 1H), 3.93 (s, 3H), 3.91 (s, 3H), 2.68 (s, 3H). LC-MS [M + H]⁺ 350.1491 |
| 171 | 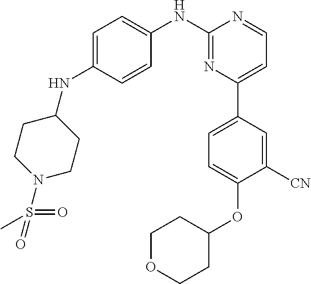 [SMILES: CS(=O)(=O)N1CCC(Nc2ccc(Nc3nccc(-c4ccc(OC5CCOCC5)c(C#N)c4)n3)cc2)CC1] | 5-{2-[(4-{[1- (methylsulfonyl) piperidin-4- yl]amino}phenyl) amino]pyrimidin-4-yl}- 2-(tetrahydro-2H- pyran-4- yloxy)benzonitrile | ¹H NMR (CDCl₃) δ 8.39 (d, 1H), 8.32 (d, 1H), 8.20 (dd, 1H), 7.45 (d, 2H), 7.08 (d, 1H), 7.02 (d, 1H), 6.70 (d, 2H), 4.77 (m, 1H), 4.04 (m, 2H), 3.78 (m, 2H), 3.66 (m, 2H), 3.43 (m, 1H), 2.92 (m, 2H), 2.84 (s, 3H), 2.20, (m, 2H), 2.09, (m, 2H), 1.94, (m, 2H), 1.58, (m, 2H). LC- MS [M + H]⁺ 549.2267 |
| 172 | 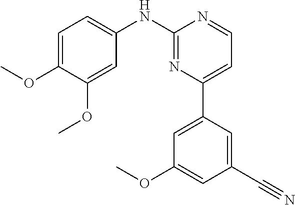 [SMILES: COc1cc(C#N)cc(-c2ccnc(Nc3ccc(OC)c(OC)c3)n2)c1] | 3-{2-[(3,4- Dimethoxyphenyl) amino]pyrimidin-4-yl}- 5- methoxybenzonitrile | ¹H NMR (CDCl₃) δ 8.50 (d, 1H), 7.94-7.93 (m, 1H), 7.85-7.84 (m, 1H), 7349 (d, 1H), 7.25-7.23 (m, 1H), 7.23 (s, 1H), 7.09 (d, 1H), 7.05-7.02 (m, 1H), 6.88 (d, 1H), 3.95 (s, 3H), 3.91 (s, 3H), 3.89 (s, 3H). LC-MS [M + H]⁺ 363.1518 |
| 173 | 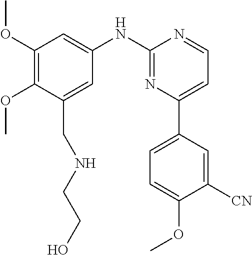 [SMILES: COc1ccc(-c2ccnc(Nc3cc(CNCCO)c(OC)c(OC)c3)n2)cc1C#N] | 5-{2-[(3-{[(2- Hydroxyethyl)amino] methyl}-4,5- dimethoxyphenyl) amino]pyrimidin-4-yl}- 2- methoxybenzonitrile | ¹H NMR (DMSO-d₆) δ 9.75 (s, 1H), 8.72 (bs, 1H), 8.58-8.52 (m, 2H), 8.51-8.49 (m, 1H), 7.76 (s, 1H), 7.49-7.40 (m, 3H), 5.25-5.22 (m, 1H), 4.10 (s, 2H), 4.02 (s, 3H), 3.89 (s, 3H), 3.79 (s, 3H), 3.70- 3.66 (m, 2H). LC-MS [M + H]⁺ 436.1981 |
| 174 | 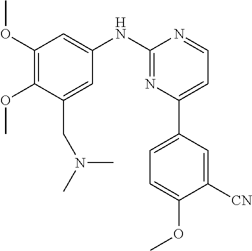 [SMILES: COc1ccc(-c2ccnc(Nc3cc(CN(C)C)c(OC)c(OC)c3)n2)cc1C#N] | 5-[2-({3- [(Dimethylamino) methyl]-4,5- dimethoxyphenyl} amino)pyrimidin-4-yl]- 2- methoxybenzonitrile | ¹H NMR (MeOH-d₄) δ 8.52 (s, 1H), 8.47 (d, 1H), 8.41-8.38 (m, 1H), 7.73 (d, 1H), 7.37 (d, 1H), 7.37- 7.31 (m, 2H), 4.32 (s, 2H), 4.04 (s, 3H), 3.96 (s, 3H), 3.93 (s, 3H), 2.89 (s, 6H). LC-MS [M + H]⁺ 420.2037 |
| 175 | 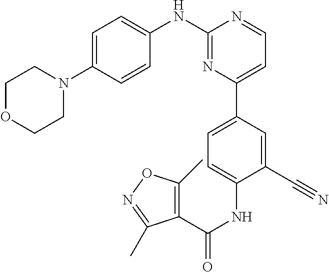 [SMILES: Cc1noc(C)c1C(=O)Nc1ccc(-c2ccnc(Nc3ccc(N4CCOCC4)cc3)n2)cc1C#N] | N-[2-cyano-4-(2-{[4- (morpholin-4- yl)phenyl]amino} pyrimidin-4-yl)phenyl]- 3,5-dimethyl-1,2- oxazole-4- carboxamide | ¹H NMR (MeOH-d₄) δ 8.50 (d, 1H), 7.94 (d, 1H), 7.87-7.83 (m, 1H), 7.58 (d, 1H), 7.19-7.17 (m, 2H), 7.11-7.07 (m, 2H), 6.83 (d, 1H), 3.87-3.84 (m, 4H), 3.25-3.22 (m, 4H), 2.34 (s, 3H), 2.21 (s, 3H). LC-MS [M + H]⁺ 496.2289 |
| 176 | 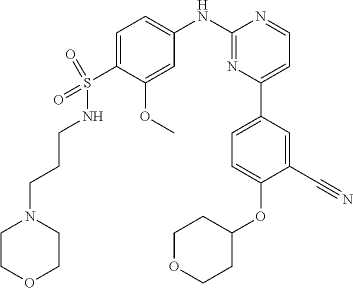 [SMILES: COc1cc(Nc2nccc(-c3ccc(OC4CCOCC4)c(C#N)c3)n2)ccc1S(=O)(=O)NCCCN1CCOCC1] | 4-({4-[3-cyano-4- (tetrahydro-2H- pyran-4- yloxy)phenyl] pyrimidin-2-yl} amino)-2- methoxy-N-[3- (morpholin-4- yl)propyl] benzenesulfonamide] | ¹H NMR (CDCl₃) δ 8.54 (d, 1H); 8.4 (s, 1H); 8.21 (d, 1H ); 7.96 (s, 1H); 7.84 (d, 1H); 7.73 (s, 1H); 7.19 (d, 1H); 7.11 (d, 1H); 7.06 (d, 1H); 4.79 (m, 1H); 4.07-3.99 (m, 5H); 3.73-3.65 (m, 6H); 2.98 (t, 2H); 2.43-2.40 (m, 6H); 2.15-2.08 (m, 2H); 1.97-1.91 (m, 2H); 1.70 (p, 2H). LC-MS [M + H]⁺ 609.2458 |
| 177 | 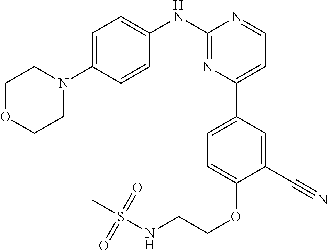 [SMILES: CS(=O)(=O)NCCOc1ccc(-c2ccnc(Nc3ccc(N4CCOCC4)cc3)n2)cc1C#N] | N-{2-[2-cyano-4-(2- {[4-(morpholin-4- yl)phenyl]amino} pyrimidin-4- yl)phenoxy]ethyl} methanesulfonamide | ¹H NMR (DMSO-d₆) δ 9.75 (br s, 1H), 8.55-8.53 (m, 2H), 8.49-8.46 (m, 1H), 7.7 (br s, 2H), 7.48-7.40 (m, 3H), 7.26 (br s, 1H), 4.31 (t, 2H), 3.87 (br s, 4H), 3.45-3.40 (m, 2H), 3.28 (br s, 4H), 3.00 (s, 3H). LC-MS [M + H]⁺ 495.1809 |
| 178 | 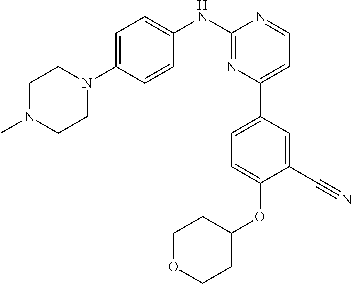 | 5-(2-{[4-(4- methylpiperazin-1- yl)phenyl]amino} pyrimidin-4-yl)-2- (tetrahydro-2H- pyran-4- yloxy)benzonitrile | ¹H NMR (MeOH-d₄) δ 8.50-8.48 (d, 1H), 8.41-8.38 (m, 2H), 7.63- 7.60 (m, 2H), 7.40 (d, 1H), 7.32- 7.30 (m, 1H), 7.41 (d, 2H), 4.02- 4.00 (m, 3H), 3.83-3.80 (m, 2H), 3.70-3.60 (m, 5H), 3.12-3.10 (m, 3H), 3.00 (s, 3H), 2.15-2.10 (m, 2H), 1.89-1.80 (m, 2H). LC-MS [M + H]⁺ 481.2499 |
| 179 | 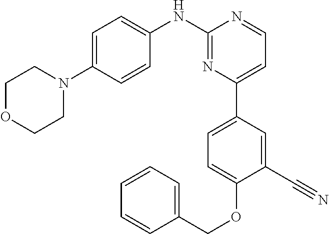 [SMILES: N#Cc1cc(-c2ccnc(Nc3ccc(N4CCOCC4)cc3)n2)ccc1OCc1ccccc1] | 2-(Benzyloxy)-5-(2- {[4-(morpholin-4- yl)phenyl]amino} pyrimidin-4- yl)benzonitrile | ¹H NMR (DMSO-d₆) δ 9.49 (s, 1H), 8.54-8.44 (m, 3H), 7.65 (d, 1H), 7.54-7.38 (m, 7H), 6.96 (d, 2H), 5.40 (s, 2H), 3.77-3.74 (m, 4H), 3.09-3.06 (m, 4H). LC-MS [M + H]⁺ 464.2050 |
| 180 | 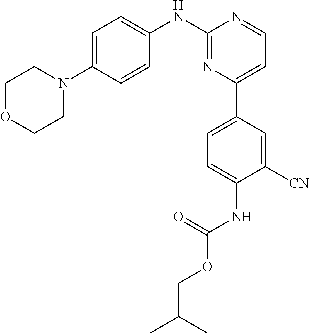 [SMILES: CC(C)COC(=O)Nc1ccc(-c2ccnc(Nc3ccc(N4CCOCC4)cc3)n2)cc1C#N] | 2-methylpropyl [2- cyano-4-(2-{[4- (morpholin-4- yl)phenyl]amino} pyrimidin-4- yl)phenyl]carbamate | ¹H NMR (CDCl₃) δ 8.44 (m, 2H), 8.32 (d, 1H), 8.24 (dd, 1H), 7.54 (dd, 2H), 7.31 (s, 1H), 7.095 (s, 1H), 7.05 (d, 1H), 6.96 (dd, 2H), 4.02 (d, 2H), 3.89 (m, 4H), 3.15 (m, 4H), 2.04 (m, 1H), 1.00 (d, 6H) LC-MS [M + H]⁺ 473.2273 |
| 181 | 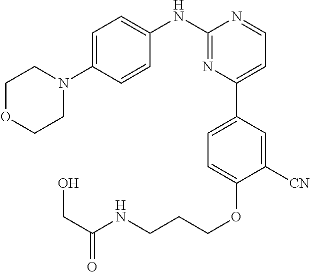 [SMILES: N#Cc1cc(-c2ccnc(Nc3ccc(N4CCOCC4)cc3)n2)ccc1OCCCNC(=O)CO] | N-{3-[2-cyano-4-(2- {[4-(morpholin-4- yl)phenyl]amino} pyrimidin-4- yl)phenoxy]propyl}- 2-hydroxyacetamide | ¹H NMR (DMSO-d₆) δ 9.46 (s, 1H), 8.51-8.44 (m, 3H), 7.99-7.95 (m, 1H), 7.65-7.61 (m, 2H), 7.41-7.38 (m, 2H), 6.93 (d, 2H), 5.49 (br s, 1H), 4.24 (t, 2H), 3.80 (s, 2H), 3.76-3.73 (m, 4H), 3.35 (s, 3H), 3.34-3.29 (m, 2H), 3.06-3.03 (m, 4H), 2.09-1.93 (m, 2H). LC-MS [M + H]⁺ 489.2259 |
| 182 | 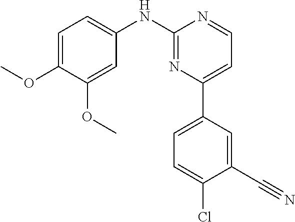 [SMILES: COc1ccc(Nc2nccc(-c3ccc(Cl)c(C#N)c3)n2)cc1OC] | 2-Chloro-5-{2-[(3,4- dimethoxyphenyl) amino]pyrimidin-4- yl}benzonitrile | ¹H NMR (DMSO-d₆) δ 9.62 (d, 1H), 8.72 (d, 1H), 8.60 (d, 1H), 8.51-8.47 (m, 1H), 7.95 (d, 1H), 7.64 (s, 1H), 7.50 (d, 1H), 7.22- 7.18 (m, 1H), 6.91 (d, 1H), 3.80 (s, 3H), 3.73 (s, 3H). LC-MS [M + H]⁺ 367.0850 |
| 183 | 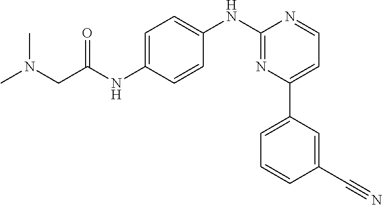 [SMILES: CN(C)CC(=O)Nc1ccc(Nc2nccc(-c3cccc(C#N)c3)n2)cc1] | N-(4-{[4-(3- Cyanophenyl) pyrimidin-2- yl]amino}phenyl)- N~2~,N~2~- dimethylglycinamide] | ¹H NMR (CDCl₃) δ 9.11 (s, 1H), 8.53 (d, 1H), 8.37-8.36 (m, 1H), 8.31-8.28 (m, 1H), 7.79-7.77 (m, 1H), 7.66-7.60 (m, 5H), 7.22 (s, 1H), 7.14 (d, 1H). LC-MS [M + H]⁺ 373.1764 |
| 184 | 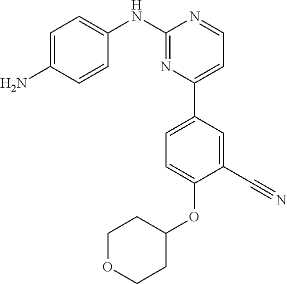 [SMILES: N#Cc1cc(-c2ccnc(Nc3ccc(N)cc3)n2)ccc1OC1CCOCC1] | 5-{2-[(4- Aminophenyl)amino] pyrimidin-4-yl}-2- (tetrahydro-2H- pyran-4- yloxy)benzonitrile | ¹H NMR (DMSO-d₆) δ 9.18 (s, 1H), 8.49 (d, 1H), 8.43 (d, 1H), 8.40 (dd, 1H), 7.53 (d, 1H), 7.36-7.30 (m, 2H), 7.32 (d, 1H), 6.58-6.54 (m, 2H), 4.93 (sept, 1H), 1.82 (br s, 2H), 3.92-3.82 (m, 2H), 3.55 (ddd, 2H), 2.10-2.00 (m, 2H), 1.75-1.62 (m, 2H); LC-MS [M + H]⁺ 388.1763 |
| 185 | 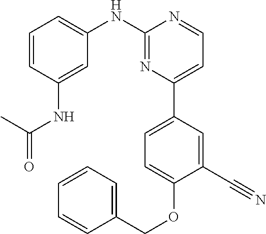 [SMILES: CC(=O)Nc1cccc(Nc2nccc(-c3ccc(OCc4ccccc4)c(C#N)c3)n2)c1] | N-[3-({4-[4- (Benzyloxy)-3- cyanophenyl] pyrimidin-2- yl}amino)phenyl] acetamide | LC-MS [M + H]⁺ 436.1930 |
| 186 | 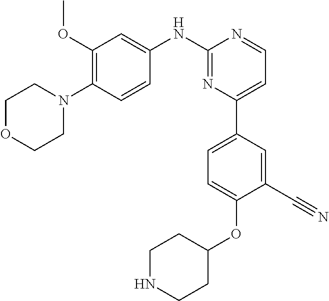 [SMILES: COc1cc(Nc2nccc(-c3ccc(OC4CCNCC4)c(C#N)c3)n2)ccc1N1CCOCC1] | 5-(2-{[3-methoxy-4- (morpholin-4- yl)phenyl]amino} pyrimidin-4-yl)-2- (piperidin-4- yloxy)benzonitrile | LC-MS [M + H]⁺ 487.3012 |
| 187 | 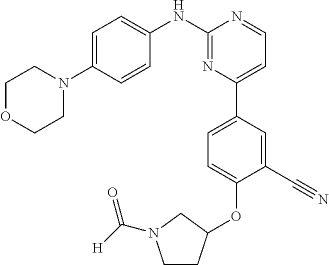 [SMILES: N#Cc1cc(-c2ccnc(Nc3ccc(N4CCOCC4)cc3)n2)ccc1OC1CCN(C=O)C1] | 2-[(1- formylpyrrolidin-3- yl)oxy]-5-(2-{[4- (morpholin-4- yl)phenyl]amino} pyrimidin-4- yl)benzonitrile | ¹H NMR (CDCl₃) δ 8.45-8.43 (m, 1H), 8.33-8.23 (m, 3H), 7.56-7.53 (m, 2H), 7.12-6.95 (m, 5H), 5.20- 5.13 (m, 1H), 3.93-3.82 (m, 6H), 3.79-3.66 (m, 4H), 3.21-3.10 (m, 6H), 2.43-2.25 (m, 2H). LC-MS [M + H]⁺ 471.2052 |
| 188 | 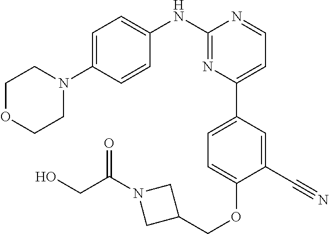 [SMILES: N#Cc1cc(-c2ccnc(Nc3ccc(N4CCOCC4)cc3)n2)ccc1OCC1CN(C(=O)CO)C1] | 2-{[1- (hydroxyacetyl) azetidin-3-yl] methoxy}-5- (2-{[4-(morpholin-4- yl)phenyl]amino} pyrimidin-4- yl)benzonitrile | ¹H NMR (DMSO-d₆) δ 9.51 (br s, 1H), 8.53-8.45 (m, 3H), 7.65 (d, 2H), 7.46 (d, 1H), 7.41 (d, 1H), 7.00-6.94 (m, 2H), 4.41 (d, 2H), 4.33 (t, 1H), 4.08-4.01 (m, 2H), 3.91 (d, 2H), 3.76-3.74 (m, 5H), 3.17-3.07 (m, 5H).OF LC-MS [M + H]⁺ 501.2253 |
| 189 | 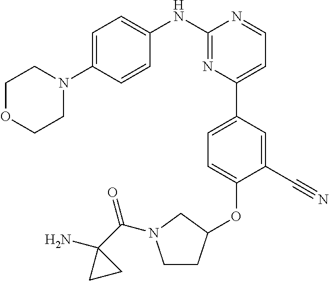 [SMILES: N#Cc1cc(-c2ccnc(Nc3ccc(N4CCOCC4)cc3)n2)ccc1OC1CCN(C(=O)C2(N)CC2)C1] | 2-({1-[(1- aminocyclopropyl) carbonyl]pyrrolidin-3- yl}oxy)-5-(2-{[4- (morpholin-4- yl)phenyl]amino} pyrimidin-4- yl)benzonitrile | ¹H NMR (DMSO-d₆) δ 9.57 (s, 1H), 8.67 (s, 2H), 8.55-8.46 (m, 3H), 7.68 (d, 2H), 7.54 (d, 1H), 7.44 (d, 1H), 7.03 (d, 2H), 5.38 (apparent s, 1H), 3.85-3.48 (m, 8H), 3.17-3.12 (m, 4H), 2.30-2.18 (m, 2H), 1.52- 1.22 (m, 4H). LC-MS [M + H]⁺ 526.2438 |
| 190 | 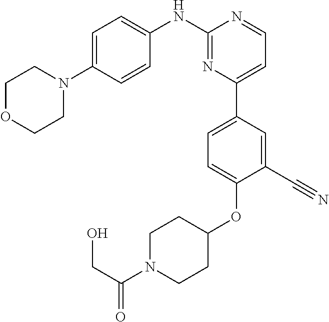 [SMILES: N#Cc1cc(-c2ccnc(Nc3ccc(N4CCOCC4)cc3)n2)ccc1OC1CCN(C(=O)CO)CC1] | 2-{[1- (Hydroxyacetyl) piperidin-4-yl] oxy}-5-(2- {[4-(morpholin-4- yl)phenyl]amino} pyrimidin-4- yl)benzonitrile | ¹H NMR (CDCl₃) δ 8.45 (d, 1H), 8.32 (d, 1H), 8.26-8.22 (m, 1H), 7.55-7.53 (m, 2H), 7.09 (d, 1H), 7.05 (s, 1H), 7.02 (d, 1H), 6.97- 6.95 (m, 2H), 4.89-4.85 (m, 1H), 4.21 (d, 2H), 4.10-4.06 (m, 1H), 3.90-3.87 (m, 4H), 3.65-3.59 (m, 3H), 3.35-3.29 (m, 1H), 3.16-3.13 (m, 4H), 2.07-1.95 (m, 4H). LC- MS [M + H]⁺ 512.2428 |
| 191 | 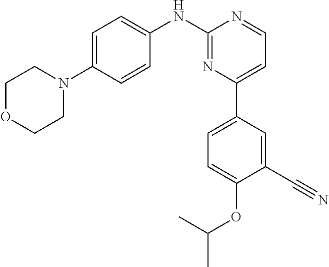 [SMILES: CC(C)Oc1ccc(-c2ccnc(Nc3ccc(N4CCOCC4)cc3)n2)cc1C#N] | 5-(2-{[4-(Morpholin- 4- yl)phenyl]amino} pyrimidin-4-yl)-2- (propan-2- yloxy)benzonitrile | ¹H NMR (DMSO-d₆) δ 9.45 (s, 1H), 8.49 (d, 1H), 8.48 (d, 1H), 8.45- 8.42 (m, 1H), 7.65-7.62 (m, 2H), 7.45 (d, 1H), 7.38 (d, 1H), 6.94- 6.91 (m, 2H), 4.95-4.89 (m, 1H), 3.76-3.73 (m, 4H), 3.06-3.03 (m, 4H), 1.37 (d, 6H). LC-MS [M + H]⁺ 416.2055 |
| 192 | 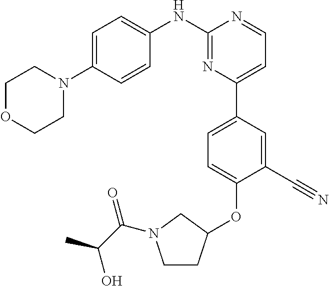 [SMILES: C[C@H](O)C(=O)N1CCC(Oc2ccc(-c3ccnc(Nc4ccc(N5CCOCC5)cc4)n3)cc2C#N)C1] | 2-({1-[(2S)-2- hydroxypropanoyl] pyrrolidin-3-yl} oxy)-5- (2-{[4-(morpholin-4- yl)phenyl]amino} pyrimidin-4- yl)benzonitrile | LC-MS [M + H]⁺ 515.2344 |
| 193 | 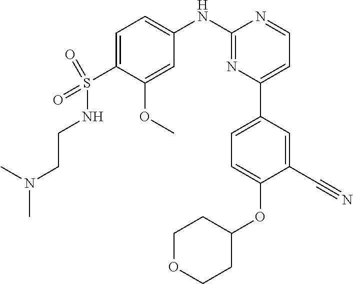 [SMILES: COc1cc(Nc2nccc(-c3ccc(OC4CCOCC4)c(C#N)c3)n2)ccc1S(=O)(=O)NCCN(C)C] | 4-({4-[3-cyano-4- (tetrahydro-2H- pyran-4- yloxy)phenyl] pyrimidin-2-yl} amino)-N- [2- (dimethylamino) ethyl]-2- methoxybenzene- sulfonamide | LC-MS [M + H]⁺ 553.2089 |
| 194 | 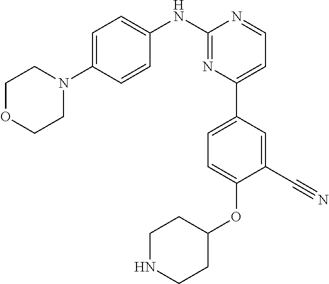 [SMILES: N#Cc1cc(-c2ccnc(Nc3ccc(N4CCOCC4)cc3)n2)ccc1OC1CCNCC1] | 5-(2-{[4-(Morpholin- 4- yl)phenyl]amino} pyrimidin-4-yl)-2- (piperidin-4- yloxy)benzonitrile | ¹H NMR (DMSO-d₆) δ 8.55-8.45 (m, 3H), 7.68 (d, 1H), 7.56 (d, 1H), 7.43 (d, 1H), 7.21 (d, 1H), 7.05- 7.01 (m, 3H), 5.04-4.99 (m, 1H), 3.79-3.73 (m, 6H), 3.25-3.11 (m, 8H), 2.99-2.92 (m, 1H), 2.22-1.90 (m, 4H). LC-MS [M + H]⁺ 457.2419 |
| 195 | 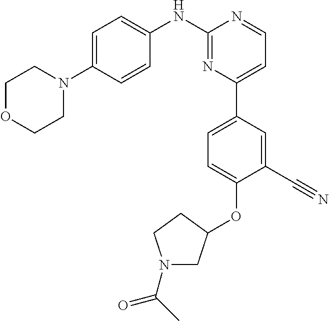 [SMILES: CC(=O)N1CCC(Oc2ccc(-c3ccnc(Nc4ccc(N5CCOCC5)cc4)n3)cc2C#N)C1] | 2-[(1- Acetylpyrrolidin-3- yl)oxy]-5-(2-{[4- (morpholin-4- yl)phenyl]amino} pyrimidin-4- yl)benzonitrile | ¹H NMR (CDCl₃) δ 8.33-8.24 (m, 3H), 7.71-7.69 (m, 2H), 7.24-7.10 (m, 4H), 5.23-5.16 (m, 1H), 4.04- 3.97 (m, 4H), 3.95-3.65 (m, 4H), 3.36-3.32 (m, 4H), 2.55-2.28 (m, 2H), 2.16 (s, 3H). Rotamers. LC- MS [M + H]⁺ 485.2196 |
| 196 | 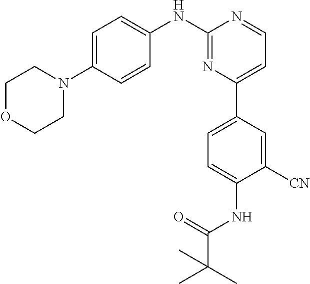 [SMILES: CC(C)(C)C(=O)Nc1ccc(-c2ccnc(Nc3ccc(N4CCOCC4)cc3)n2)cc1C#N] | N-[2-cyano-4-(2-{[4- (morpholin-4- yl)phenyl]amino} pyrimidin-4-yl)phenyl]- 2,2- dimethylpropanamide | ¹H NMR (CDCl₃) δ 8.63 (d, 1H), 8.46 (d, 1H), 8.34 (d, 1H), 8.26- 8.23 (m, 1H), 8.12 (s, 1H), 7.56- 7.52 (m, 2H), 7.26 (s, 1H), 7.06 (d, 1H), 6.98-6.94 (m, 2H), 3.90-3.88 (m, 4H), 3.17-3.14 (m, 4H), 1.39 (s, 9H). LC-MS [M + H]⁺ 457.2311 |
| 197 | 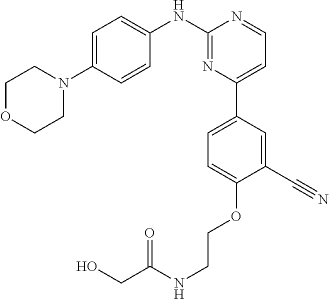 [SMILES: N#Cc1cc(-c2ccnc(Nc3ccc(N4CCOCC4)cc3)n2)ccc1OCCNC(=O)CO] | N-{2-[2-cyano-4-(2- {[4-(morpholin-4- yl)phenyl]amino} pyrimidin-4- yl)phenoxy]ethyl}-2- hydroxyacetamide | ¹H NMR (DMSO-d₆) δ 9.54 (s, 1H), 8.52-8.44 (m, 3H), 7.02-7.99 (m, 1H), 7.67 (d, 1H), 7.47 (d, 1H), 7.42 (d, 1H), 7.26 (s, 1H), 7.15 (s, 1H), 7.02-6.99 (m, 2H), 4.31 (t, 2H), 3.84 (s, 2H), 3.78-3.75 (m, 4H), 3.58-3.54 (m, 2H), 3.13-3.10 (m, 4H). LC-MS [M + H]⁺ 475.2081 |
| 198 | 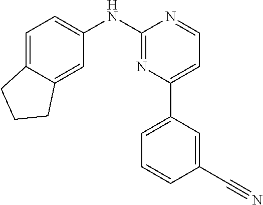 [SMILES: N#Cc1cccc(-c2ccnc(Nc3ccc4c(c3)CCC4)n2)c1] | 3-[2-(2,3-Dihydro- 1H-inden-5- ylamino)pyrimidin- 4-yl]benzonitrile | ¹H NMR (CDCl₃) δ 8.51 (d, 1H), 8.40-8.39 (m, 1H), 8.28-8.25 (m, 1H), 7.78-7.75 (m, 1H), 7.63-7.56 (m, 2H), 7.37-7.35 (m, 1H), 7.27 (br s, 1H), 7.22 (d, 1H), 7.11 (d, 1H), 2.98-2.88 (m, 4H), 2.15-2.07 (m, 2H). LC-MS [M + H]⁺ 313.1449 |
| 199 | 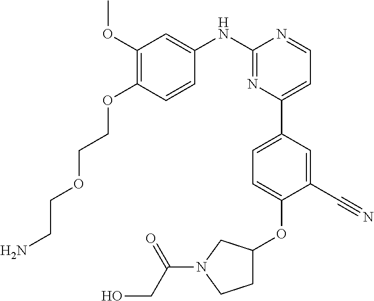 [SMILES: COc1cc(Nc2nccc(-c3ccc(OC4CCN(C(=O)CO)C4)c(C#N)c3)n2)ccc1OCCOCCN] | 5-[2-({4-[2-(2- aminoethoxy)ethoxy]- 3- methoxyphenyl} amino)pyrimidin- 4-yl]-2-{[1- (hydroxyacetyl) pyrrolidin-3- yl]oxy}benzonitrile | ¹H NMR (MeOH-d₄) δ 8.51 (d, 1H), 8.44 (d, 1H), 8.40 (dd, 1H), 7.54 (d, 1H), 7.40 (d, 1H), 7.30 (d, 1H,) 7.14 (dd, 1H), 7.0(d, 1H), 4.24 (s, 1H), 4.18-4.17 (m, 2H), 4.15- 4.13 (m, 2H), 3.90 (s, 3H), 3.84- 3.80 (m, 3H), 3.80-3.67 (m, 2H), 3.60 (t, 1H), 3.26 (t, 2H), 2.43-2.40 (m, 2H), 2.30-2.27 (m, 2H). LC- MS [M + H]⁺ 549.2332 |
| 200 | 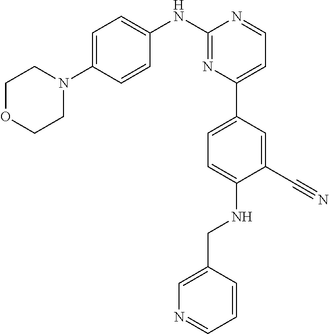 [SMILES: N#Cc1cc(-c2ccnc(Nc3ccc(N4CCOCC4)cc3)n2)ccc1NCc1cccnc1] | 5-(2-{[4-(Morpholin- 4- yl)phenyl]amino} pyrimidin-4-yl)-2- [(pyridin-3- ylmethyl)amino] benzonitrile | LC-MS [M + H]⁺ 464.2187 |
| 201 | 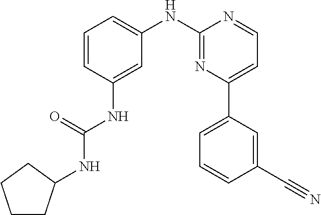 [SMILES: N#Cc1cccc(-c2ccnc(Nc3cccc(NC(=O)NC4CCCC4)c3)n2)c1] | 1-(3-{[4-(3- Cyanophenyl) pyrimidin-2- yl]amino}phenyl)-3- cyclopentylurea | ¹H NMR (DMSO-d₆) δ 9.69 (s, 1H), 8.67-8.59 (m, 3H), 8.25 (s, 1H), 8.03-8.01 (m, 2H), 7.78-7.73 (m, 1H), 7.53 (d, 1H), 7.26-7.23 (m, 1H), 7.15-7.11 (m, 1H), 7.00-6.98 (m, 1H), 6.13 (d, 1H), 4.03-3.95 (m, 1H), 2.08-1.80 (m, 2H), 1.66- 1.53 (m, 4H), 1.40-1.34 (m, 2H). LC-MS [M + H]⁺ 399.1969 |
| 202 | 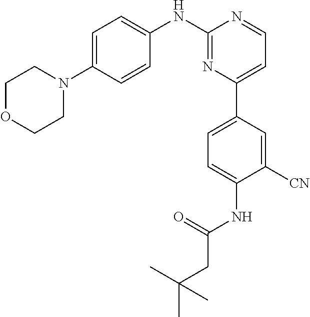 [SMILES: CC(C)(C)CC(=O)Nc1ccc(-c2ccnc(Nc3ccc(N4CCOCC4)cc3)n2)cc1C#N] | N-[2-cyano-4-(2-{[4- (morpholin-4- yl)phenyl]amino} pyrimidin-4-yl)phenyl]- 3,3- dimethylbutanamide | ¹H NMR (CDCl₃) δ 8.61 (d, 1H), 8.45 (d, 1H), 8.33 (d, 1H), 8.24 (dd, 1H), 7.67 (s, 1H), 7.56-7.52 (m, 2H), 7.16 (s, 1H), 7.05 (d, 1H), 6.98- 6.96 (m, 2H), 3.91-3.88 (m, 4H), 3.17-3.13 (m, 4H), 2.36 (s, 2H), 1.15 (s, 9H). LC-MS [M + H]⁺ 471.2494 |
| 203 | 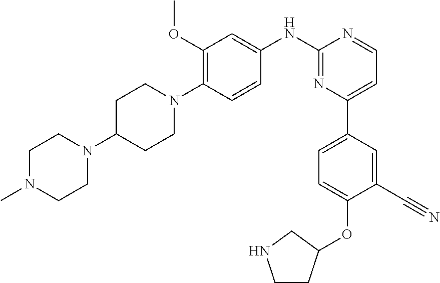 [SMILES: COc1cc(Nc2nccc(-c3ccc(OC4CCNC4)c(C#N)c3)n2)ccc1N1CCC(N2CCN(C)CC2)CC1] | 5-[2-({3-methoxy-4- [4-(4- methylpiperazin-1- yl)piperidin-1- yl]phenyl}amino) pyrimidin-4-yl]-2- (pyrrolidin-3- yloxy)benzonitrile | LC-MS [M + H]⁺ 469.40 |
| 204 | 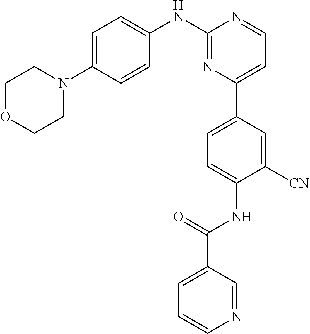 [SMILES: N#Cc1cc(-c2ccnc(Nc3ccc(N4CCOCC4)cc3)n2)ccc1NC(=O)c1cccnc1] | N-[2-cyano-4-(2-{[4- (morpholin-4- yl)phenyl]amino} pyrimidin-4- yl)phenyl]pyridine-3- carboxamide | ¹H NMR (CDCl₃) δ 9.20 (s, 1H), 8.81 (s, 1H), 8.45 (m, 2H), 8.37 (m, 2H), 8.29 (d, 1H), 7.59 (m, 3H), 7.19 (d, 1H), 7.06 (d, 2H), 3.93 (m, 4H), 3.22 (m, 4H). LC-MS [M + H]⁺ 478.1973 |
| 205 | 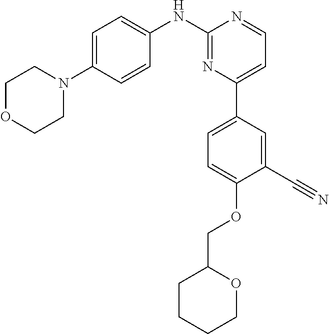 [SMILES: N#Cc1cc(-c2ccnc(Nc3ccc(N4CCOCC4)cc3)n2)ccc1OCC1CCCCO1] | 5-(2-{[4-(Morpholin- 4- yl)phenyl]amino} pyrimidin-4-yl)-2- (tetrahydro-2H- pyran-2- ylmethoxy) benzonitrile | ¹H NMR (DMSO-d₆) δ 9.47 (s, 1H), 8.50 (d, 1H), 8.48 (d, 1H), 8.46- 8.42 (m, 1H), 7.63 (d, 2H), 7.44 (d, 1H), 7.39 (d, 1H), 6.93 (d, 2H), 4.24-4.16 (m, 2H), 3.94-3.89 (m, 1H), 3.76-3.69 (m, 5H), 3.45-3.38 (m, 1H), 3.35 (s, 2H), 3.05-3.03 (m, 4H), 1.88-1.82 (m, 1H), 1.72-1.67 (m, 1H), 1.56-1.34 (m, 4H). LC- MS [M + H]⁺ 472.2490 |
| 206 | 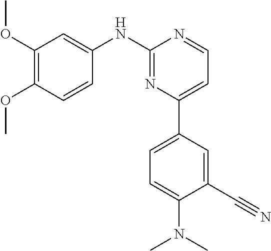 [SMILES: COc1ccc(Nc2nccc(-c3ccc(N(C)C)c(C#N)c3)n2)cc1OC] | 5-{2-[(3,4- Dimethoxyphenyl) amino]pyrimidin-4-yl}- 2- (dimethylamino) benzonitrile | ¹H NMR (CDCl₃) δ 8.40 (d, 1H), 8.28 (d, 1H), 8.09-8.06 (m, 1H), 7.53 (d, 1H), 7.11 (br s, 1H), 7.03- 7.00 (m, 2H), 6.90-6.86 (m, 2H), 3.96 (s, 3H), 3.89 (s, 3H), 3.21 (s, 6H). LC-MS [M + H]⁺ 376.1853 |
| 207 | 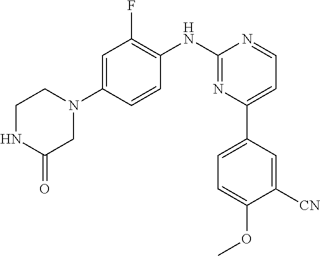 [SMILES: COc1ccc(-c2ccnc(Nc3ccc(N4CCNC(=O)C4)cc3F)n2)cc1C#N] | 5-(2-{[2-Fluoro-4-(3- oxopiperazin-1- yl)phenyl]amino} pyrimidin-4-yl)-2- methoxybenzonitrile | ¹H NMR (CDCl₃) δ 8.64-8.52 (m, 2H), 8.14-8.11 (m, 1H), 8.08-8.05 (m, 1H), 8.01 (d, 1H), 7.35-7.29 (m, 2H), 7.07 (d, 1H), 6.88 (d, 2H), 4.05-4.03 (m, 2H), 3.99-3.97 (m, 2H), 3.88 (s, 3H), 3.68-3.65 (m, 1H), 3.45-3.42 (m, 1H). LC-MS [M + H]⁺ 419.1444 |
| 208 | 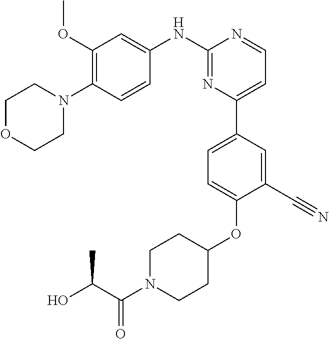 [SMILES: COc1cc(Nc2nccc(-c3ccc(OC4CCN(C(=O)[C@H](C)O)CC4)c(C#N)c3)n2)ccc1N1CCOCC1] | 2-({1-[(2S)-2- hydroxypropanoyl] piperidin-4-yl}oxy)-5- (2-{[3-methoxy-4- (morpholin-4- yl)phenyl]amino} pyrimidin-4- yl)benzonitrile | ¹H NMR (MeOH-d₄) δ 8.57 (d, 1H), 8.54 (d, 1H), 8.42 (dd, 1H), 8.06 (s, 1H), 7.47-7.35 (m, 4H), 4.09 (s, 3H), 4.06-4.04 (m, 4H), 3.55-3.50 (m, 4H), 3.43-3.40 (m, 4H), 2.90 (s, 2H), 2.15-2.10 (m, 2H), 2.07-2.00 (m, 2H), 1.33-1.34 (d, 3H). LC-MS [M + H]⁺ 559.2739 |
| 209 | 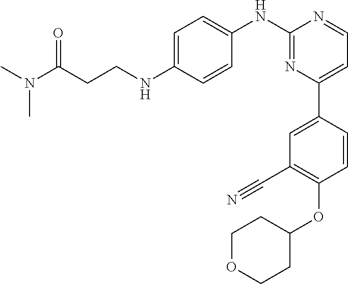 [SMILES: CN(C)C(=O)CCNc1ccc(Nc2nccc(-c3ccc(OC4CCOCC4)c(C#N)c3)n2)cc1] | N~3~-[4-({4-[3- cyano-4-(tetrahydro- 2H-pyran-4- yloxy)phenyl] pyrimidin-2- yl}amino)phenyl]- N,N-dimethyl-beta- alaninamide | LC-MS [M + H]⁺ 487.2490 |
| 210 | 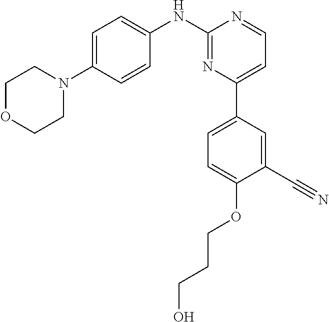 [SMILES: N#Cc1cc(-c2ccnc(Nc3ccc(N4CCOCC4)cc3)n2)ccc1OCCCO] | 2-(3- Hydroxypropoxy)-5- (2-{[4-(morpholin-4- yl)phenyl]amino} pyrimidin-4- yl)benzonitrile | ¹H NMR (CDCl₃) δ 8.32-8.27 (m, 2H), 8.20 (d, 1H), 7.64-7.61 (m, 2H), 7.19-7.15 (m, 2H), 7.08-7.05 (m, 2H), 4.37 (t, 2H), 3.96-3.92 (m, 6H), 3.26-3.23 (m, 4H), 2.20-2.10 (m, 2H). LC-MS [M + H]⁺ 432.2030 |
| 211 | 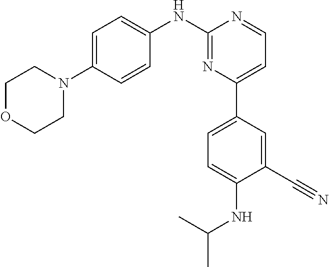 [SMILES: CC(C)Nc1ccc(-c2ccnc(Nc3ccc(N4CCOCC4)cc3)n2)cc1C#N] | 5-(2-{[4-(Morpholin- 4- yl)phenyl]amino} pyrimidin-4-yl)-2- (propan-2- ylamino)benzonitrile | ¹H NMR (DMSO-d₆) δ 9.82 (s, 1H), 8.43 (d, 1H), 8.35-8.22 (m, 2H), 7.74 (br s, 1H), 7.39 (d, 1H), 7.23 (d, 2H), 7.06-6.96 (m, 2H), 3.76- 3.73 (m, 4H), 3.14-3.12 (m, 4H), 1.23 (d, 6H). LC-MS [M + H]⁺ 415.2227 |
| 212 | 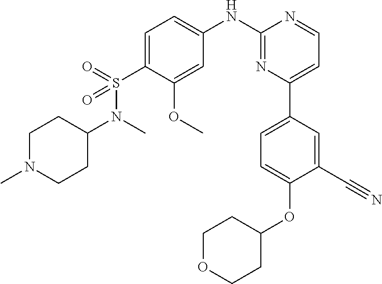 [SMILES: COc1cc(Nc2nccc(-c3ccc(OC4CCOCC4)c(C#N)c3)n2)ccc1S(=O)(=O)N(C)C1CCN(C)CC1] | 4-(4-{4-[3-cyano-4- (tetrahydro-2H- pyran-4- yloxy)phenyl] pyrimidin-2-yl} amino)-2- methoxy-N-methyl- N-(1- methylpiperidin-4- yl)benzene- sulfonamide] | ¹H NMR (DMSO-d₆) δ 8.54 (d, 1H); 8.38 (d, 1H); 8.21 (d, 1H); 7.89-7.87 (m, 2H), 7.50 (s, 1H); 7.18 (d, 1H); 7.10 (d, 1H); 7.04 (d, 1H); 4.80-4.76 (m, 1H); 4.07-4.01 (m, 5H); 3.78-3.65 (m, 2H), 2.90- 2.85 (m, 5H); 2.26 (s, 3H); 2.15- 2.07 (m, 2H); 2.02-1.90 (M, 5H); 1.84-1.74 (m, 3H); 1.57-1.54 (m, 2H). LC-MS [M + H]⁺ 593.2441 |
| 213 | 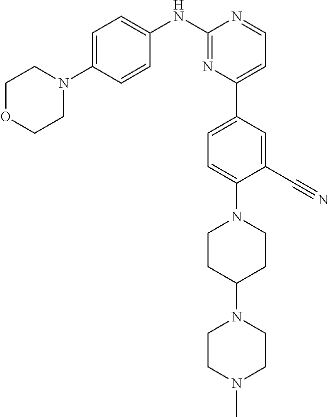 [SMILES: CN1CCN(C2CCN(c3ccc(-c4ccnc(Nc5ccc(N6CCOCC6)cc5)n4)cc3C#N)CC2)CC1] | 2-[4-(4- Methylpiperazin-1- yl)piperidin-1-yl]-5- (2-{[4-(morpholin-4- yl)phenyl]amino} pyrimidin-4- yl)benzonitrile | LC-MS [M + H]⁺ 539.3193 |
| 214 | 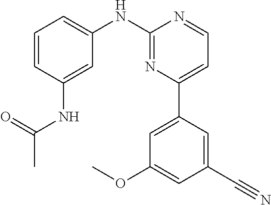 [SMILES: COc1cc(C#N)cc(-c2ccnc(Nc3cccc(NC(C)=O)c3)n2)c1] | N-(3-{[4-(3-Cyano- 5- methoxyphenyl) pyrimidin-2- yl]amino}phenyl) acetamide | ¹H NMR (DMSO-d₆) δ 9.92 (s, 1H), 9.76 (s, 1H), 8.60 (d, 1H), 8.31- 8.27 (m, 2H), 8.05-8.04 (m, 1H), 7.61-7.60 (m, 1H), 7.55 (d, 1H), 7.36-7.33 (m, 1H), 7.22-7.13 (m, 2H), 3.92 (s, 3H), 2.06 (s, 3H). LC-MS [M + H]⁺ 360.1509 |
| 215 | 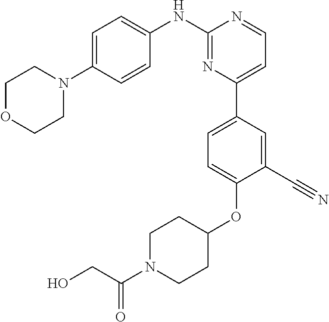 [SMILES: N#Cc1cc(-c2ccnc(Nc3ccc(N4CCOCC4)cc3)n2)ccc1OC1CCN(C(=O)CO)CC1] | 2-{[1-(3- Hydroxypropanoyl) piperidin-4-yl]oxy}-5- (2-{[4-(morpholin-4- yl)phenyl]amino} pyrimidin-4- yl)benzonitrile | ¹H NMR (MeOH-d₄) δ 8.47-8.39 (m, 4H), 7.76 (br s, 2H), 7.41-7.24 (m, 3H), 4.99-4.95 (m, 1H), 4.06- 3.62 (m, 12H), 2.67-2.64 (m, 2H), 2.14-1.83 (m, 4H). LC-MS [M + H]⁺ 529.2427 |
| 216 | AND Enantiomer 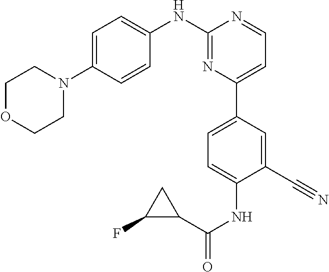 [SMILES: N#Cc1cc(-c2ccnc(Nc3ccc(N4CCOCC4)cc3)n2)ccc1NC(=O)C1C[C@@H]1F] | (2S)-N-[2-cyano-4- (2-{[4-(morpholin-4- yl)phenyl]amino} pyrimidin-4-yl)phenyl]- 2- fluorocyclopropane- carboxamide | ¹H NMR (DMSO-d₆) δ 10.8 (s, 1H), 9.51 (s, 1H), 8.57-8.43 (m, 3H), 7.86 (d, 1H), 7.64 (d, 2H), 7.42 (d, 1H), 6.93 (d, 2H), 5.02-4.84 (m, 1H), 3.76-3.73 (m, 4H), 3.06-3.04 (m, 4H), 1.65-1.59 (m, 1H), 1.32- 1.26 (m, 2H). LC-MS [M + H]⁺ 459.2037 |
| 217 | 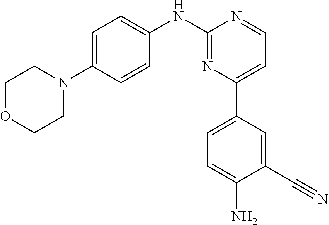 [SMILES: N#Cc1cc(-c2ccnc(Nc3ccc(N4CCOCC4)cc3)n2)ccc1N] | 2-Amino-5-(2-{[4- (morpholin-4- yl)phenyl]amino} pyrimidin-4- yl)benzonitrile | ¹H NMR (DMSO-d₆) δ 9.31 (s, 1H), 8.38 (d, 1H), 8.24 (d, 1H), 8.13- 8.10 (m, 1H), 7.63 (d, 2H), 7.23 (d, 1H), 6.93-6.87 (m, 3H), 6.65 (br s, 2H), 3.75-3.73 (m, 4H), 3.05-3.03 (m, 4H). LC-MS [M + H]⁺ 373.1816 |
| 218 | 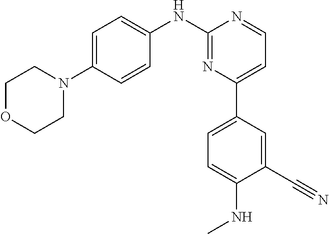 [SMILES: CNc1ccc(-c2ccnc(Nc3ccc(N4CCOCC4)cc3)n2)cc1C#N] | 2-(Methylamino)-5- (2-{[4-(morpholin-4- yl)phenyl]amino} pyrimidin-4- yl)benzonitrile | ¹H NMR (DMSO-d₆) δ 9.49 (br s, 1H), 8.28 (d, 1H), 7.46 (d, 1H), 7.40-7.30 (m, 3H), 6.83-6.76 (m, 3H), 6.38 (d, 1H), 3.78-3.73 (m, 5H), 3.06-3.02 (m, 4H), 2.80 (s, 3H). LC-MS [M + H]⁺ 387.1927 |
| 219 | 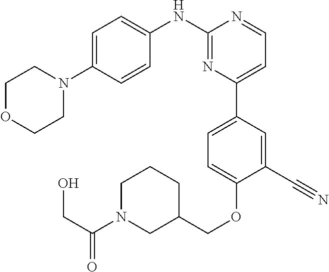 [SMILES: N#Cc1cc(-c2ccnc(Nc3ccc(N4CCOCC4)cc3)n2)ccc1OCC1CCCN(C(=O)CO)C1] | 2-{[1- (hydroxyacetyl) piperidin-3-yl]methoxy}- 5-(2-{[4-(morpholin- 4- yl)phenyl]amino} pyrimidin-4- yl)benzonitrile | ¹H NMR (DMSO-d₆) δ 9.47 (s, 1H), 8.53-8.44 (m, 3H), 7.64 (d, 2H), 7.47-7.42 (m, 1H), 7.39 (d, 1H), 6.93 (d, 2H), 4.50-4.32 (m, 1H), 4.17-4.08 (m, 5H), 3.76-3.73 (m, 4H), 3.59-3.55 (m, 1H), 3.06-3.03 (m, 4H), 2.99-2.74 (m, 1H), 2.07- 1.87 (m, 2H), 1.75-1.67 (m, 1H), 1.52-1.38 (m, 2H). LC-MS [M + H]⁺ 529.2476 |
| 220 | 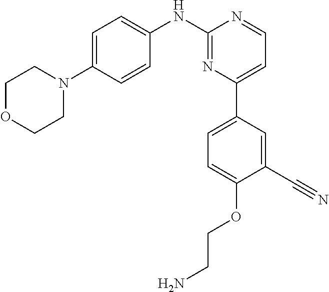 [SMILES: N#Cc1cc(-c2ccnc(Nc3ccc(N4CCOCC4)cc3)n2)ccc1OCCN] | 2-(2-aminoethoxy)-5- (2-{[4-(morpholin-4- yl)phenyl]amino} pyrimidin-4- yl)benzonitrile | ¹H NMR (DMSO-d₆) δ 9.46 (s, 1H), 8.52-8.43 (m, 3H), 7.65-7.62 (m, 2H), 7.44 (d, 1H), 7.39 (d, 1H), 6.93 (d, 2H), 4.19 (t, 2H), 3.76- 3.73 (m, 4H), 3.06-3.03 (m, 4H), 2.96 (t, 2H). LC-MS [M + H]⁺ 418.2023 |
| 221 | 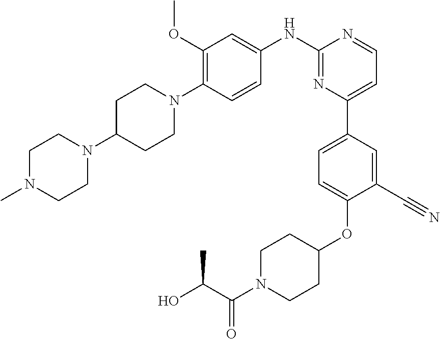 [SMILES: COc1cc(Nc2nccc(-c3ccc(OC4CCN(C(=O)[C@H](C)O)CC4)c(C#N)c3)n2)ccc1N1CCC(N2CCN(C)CC2)CC1] | 2-({1-[(2S)-2- hydroxypropanoyl] piperidin-4-yl}oxy)-5- [2-({3-methoxy-4-[4- (4-methylpiperazin- 1-yl)piperidin-1- yl]phenyl}amino) pyrimidin-4- yl]benzonitrile | LC-MS [M + H]⁺ 655.2067 |
| 222 | 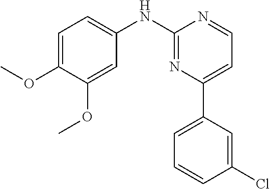 [SMILES: COc1ccc(Nc2nccc(-c3cccc(Cl)c3)n2)cc1OC] | 4-(3-Chlorophenyl)- N-(3,4- dimethoxyphenyl) pyrimidin-2-amine | ¹H NMR (CDCl₃) δ 9.22 (br s, 1H), 8.38 (d, 1H), 8.14-8.12 (m, 1H), 7.96-7.93 (m, 1H), 7.57-7.54 (m, 1H), 7.50-7.45 (m, 2H), 7.20 (d, 1H), 7.09-7.06 (m, 1H), 6.89 (d, 1H), 3.95 (s, 3H), 3.91 (s, 3H). LC-MS [M + H]⁺ 342.1011 |
| 223 | 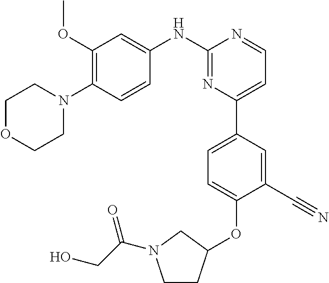 [SMILES: COc1cc(Nc2nccc(-c3ccc(OC4CCN(C(=O)CO)C4)c(C#N)c3)n2)ccc1N1CCOCC1] | 2-{[1- (hydroxyacetyl) pyrrolidin-3-yl] oxy}-5-(2- {[3-methoxy-4- (morpholin-4- yl)phenyl]amino} pyrimidin-4- yl)benzonitrile | ¹H NMR (DMSO-d₆) δ 9.86 (s, 1H), 8.59-8.58 (m, 2H), 8.49-8.46 (m, 1H), 7.86 (br s, 1H), 7.53-7.51 (m, 2H), 7.35-7.04 (m, 3H), 4.12-3.80 (m, 10H), 3.73-3.61 (m, 3H), 3.34- 3.43 (m, 1H), 3.28 (br s, 4H), 2.35- 2.15 (m, 2H). LC-MS [M + H]⁺ 531.2299 |
| 224 | 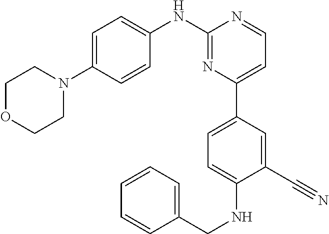 [SMILES: N#Cc1cc(-c2ccnc(Nc3ccc(N4CCOCC4)cc3)n2)ccc1NCc1ccccc1] | 2-(Benzylamino)-5- (2-{[4-morpholin-4- yl)phenyl]amino} pyrimidin-4- yl)benzonitrile | ¹H NMR (CDCl₃) δ 8.36 (d, 1H), 8.21 (d, 1H), 8.08-8.04 (m, 1H), 7.56-7.53 (m, 2H), 7.39-7.32 (m, 4H), 7.08 (s, 1H), 6.97-6.94 (m, 3H), 6.73 (d, 1H), 5.34 (t, 1H), 4.52 (d, 2H), 3.90-3.87 (m, 4H), 3.15-3.13 (m, 4H). LC-MS [M + H]⁺ 463.2135 |
| 225 | 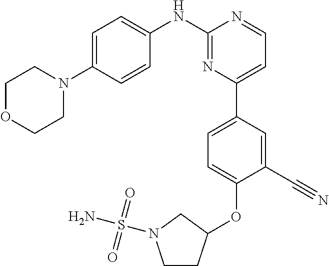 [SMILES: N#Cc1cc(-c2ccnc(Nc3ccc(N4CCOCC4)cc3)n2)ccc1OC1CCN(S(N)(=O)=O)C1] | 3-[2-cyano-4-(2-{[4- (morpholin-4- yl)phenyl]amino} pyrimidin-4- yl)phenoxy]pyrrolidine- 1-sulfonamide | ¹H NMR (CDCl₃) δ 9.57 (s, 1H), 8.54-8.51 (m, 3H), 7.68 (d, 2H), 7.46-7.42 (m, 2H), 7.02 (d, 2H), 6.92 (br s, 2H), 5.33 (br s, 1H), 3.78 (br s, 4H), 3.63-3.59 (m, 1H), 3.32-3.27 (m, 3H), 3.13 (br s, 4H), 3.39-32.30 (m, 1H), 2.12-2.08 (m, 1H). LC-MS [M + H]⁺ 522.1906 |
| 226 | 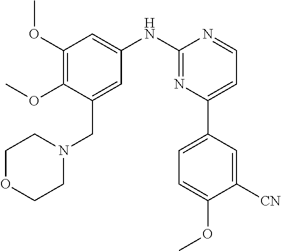 [SMILES: COc1ccc(-c2ccnc(Nc3cc(CN4CCOCC4)c(OC)c(OC)c3)n2)cc1C#N] | 5-(2-{[3,4- Dimethoxy-5- (morpholin-4- ylmethyl)phenyl] amino}pyrimidin-4-yl)- 2- methoxybenzonitrile | ¹H NMR (CDCl₃) δ 8.44 (d, 1H), 8.29-8.25 (m, 2H), 7.64 (d, 1H), 7.31 (d, 1H), 7.28 (s, 1H), 7.209 (d, 1H), 4.36 (s, 2H), 4.06 (s, 3H), 4.00-3.88 (m, 4H), 3.97 (s, 3H), 3.92 (s, 3H), 3.50 (d, 2H), 3.02- 2.97 (m, 2H). LC-MS [M + H]⁺ 462.2130 |
| 227 | 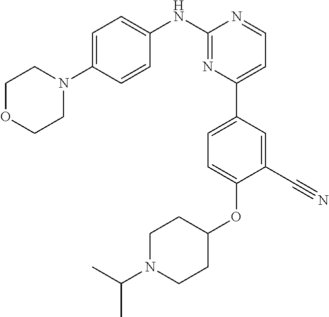 [SMILES: CC(C)N1CCC(Oc2ccc(-c3ccnc(Nc4ccc(N5CCOCC5)cc4)n3)cc2C#N)CC1] | 5-(2-{[4-(Morpholin- 4- yl)phenyl]amino} pyrimidin-4-yl)-2-{[1- (propan-2- yl)piperidin-4- yl]oxy}benzonitrile | ¹H NMR (DMSO-d₆) δ 8.46 (d, 1H), 8.34 (d, 1H), 8.30-8.27 (m, 1H), 7.55-7.52 (m, 2H), 7.14 (d, 1H), 7.06 (br s, 1H), 7.03 (d, 1H), 6.98- 6.95 (m, 2H), 3.90-3.87 (m, 4H), 3.58-3.50 (m, 1H), 3.45-3.30 (m, 2H), 3.16-3.14 (m, 4H), 3.08-2.95 (m, 1H), 2.30-2.22 (m, 2H), 1.65- 1.45 (m, 8H). LC-MS [M + H]⁺ 499.2771 |
| 228 | 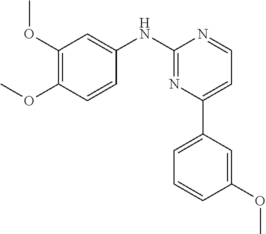 | N-(3,4- Dimethoxyphenyl)-4- (3- methoxyphenyl) pyrimidin-2-amine | ¹H NMR (CDCl₃) δ 8.44 (d, 1H), 7.67-7.61 (m, 2H), 7.54 (d, 1H), 7.42-7.38 (m, 1H), 7.15 (s, 1H), 7.12 (d, 1H), 7.06-7.02 (m, 2H), 6.87 (d, 1H), 3.94 (s, 3H), 3.89 (s, 3H), 3.88 (s, 3H). LC-MS [M + H]⁺ 338.1551 |
| 229 | 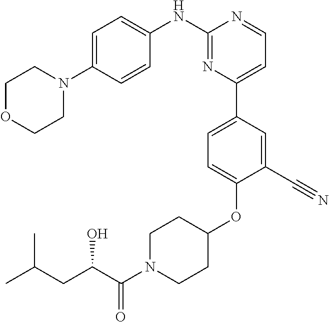 [SMILES: CC(C)C[C@H](O)C(=O)N1CCC(Oc2ccc(-c3ccnc(Nc4ccc(N5CCOCC5)cc4)n3)cc2C#N)CC1] | 2-({1-[(2S)-2- hydroxy-4- methylpentanoyl] piperidin-4-yl}oxy)-5- (2-{[4-(morpholin-4- yl)phenyl]amino} pyrimidin-4- yl)benzonitrile | ¹H NMR (DMSO-d₆) δ 9.51 (s, 1H), 8.56-8.43 (m, 3H), 7.68 (d, 2H), 7.55 (d, 1H), 7.43 (br s, 1H), 7.04 (br s, 2H), 5.05-4.96 (m, 1H), 4.38- 4.35 (m, 1H), 3.86-3.66 (m, 6H), 3.54-3.37 (m, 2H), 3.13 (br s, 4H), 2.08-1.95 (m, 2H), 1.80-1.63 (m, 3H), 1.44-1.32 (m, 3H), 0.88 (d, 6H). LC-MS [M + H]⁺ 571.3013 |
| 230 | 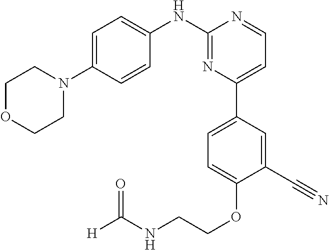 [SMILES: N#Cc1cc(-c2ccnc(Nc3ccc(N4CCOCC4)cc3)n2)ccc1OCCNC=O] | N-{2-[2-cyano-4-(2- {[4-(morpholin-4- yl)phenyl]amino} pyrimidin-4- yl)phenoxy]ethyl} formamide | ¹H NMR (DMSO-d₆) δ 8.44 (d, 1H), 8.32 (d, 1H), 8.26 (s, 1H), 8.24 (d, 1H), 7.54 (d, 2H), 7.08 (d, 2H), 7.03-6.95 (m, 3H), 6.27 (br s, 1H), 4.26 (t, 2H), 3.90-3.87 (m, 4H), 3.85-3.80 (m, 2H), 3.15 (br s, 4H). LC-MS [M + H]⁺ 445.1962 |
| 231 | 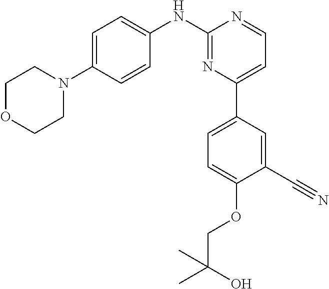 [SMILES: CC(C)(O)COc1ccc(-c2ccnc(Nc3ccc(N4CCOCC4)cc3)n2)cc1C#N] | 2-(2-Hydroxy-2- methylpropoxy)-5- (2-{[4-(morpholin-4- yl)phenyl]amino} pyrimidin-4- yl)benzonitrile | ¹H NMR (CDCl₃) δ 8.44 (d, 1H), 8.31 (d, 1H), 8.25-8.22 (m, 1H), 8.02 (s, 1H), 7.56-7.53 (m, 2H), 7.21 (s, 1H), 7.07 (d, 1H), 7.02 (d, 1H), 6.97-6.94 (m, 2H), 3.98 (s, 2H), 3.90-3.87 (m, 4H), 3.16-3.13 (m, 4H), 1.43 (s, 6H). LC-MS [M + H]⁺ 446.2107 |
| 232 | 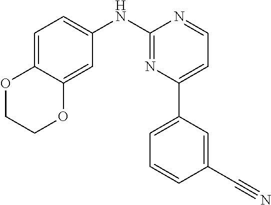 [SMILES: N#Cc1cccc(-c2ccnc(Nc3ccc4c(c3)OCCO4)n2)c1] | 3-[2-(2,3-Dihydro- 1,4-benzodioxin-6- ylamino)pyrimidin- 4-yl]benzonitrile | ¹H NMR (CDCl₃) δ 8.49 (d, 1H), 8,36-8.35 (m, 1H), 8.29-8.26 (m, 1H), 7.78-7.75 (m, 1H), 7.64-7.60 (m, 1H), 7.34 (d, 1H), 7.14 (br s, 1H), 7.10 (d, 1H), 7.02-6.99 (m, 1H), 6.87 (d, 1H), 4.31-4.25 (m, 4H). LC-MS [M + H]⁺ 331.1192 |
| 233 | 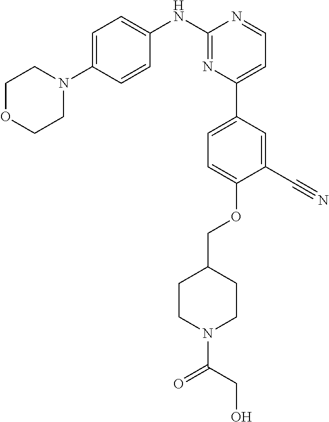 [SMILES: N#Cc1cc(-c2ccnc(Nc3ccc(N4CCOCC4)cc3)n2)ccc1OCC1CCN(C(=O)CO)CC1] | 2-{[1- (hydroxyacetyl) piperidin-4-yl] methoxy}- 5-(2-{[4-(morpholin- 4- yl)phenyl]amino} pyrimidin-4- yl)benzonitrile | ¹H NMR (CDCl₃) δ 9.47 (s, 1H), 8.52-8.44 (m, 3H), 8.18 (br s, 1H), 7.63 (d, 2H), 7.44 (d, 1H), 7.39 (d, 1H), 6.92 (d, 2H), 4.42-4.38 (m, 1H), 4.13-4.46 (m, 2H), 3.76-3.73 (m, 4H), 3.11-2.97 (m, 6H), 2.71- 2.65 (m, 1H), 2.11 (br s, 1H), 1.86- 1.80 (m, 2H), 1.32-1.18 (m, 4H). LC-MS [M + H]⁺ 529.2469 |
| 234 | 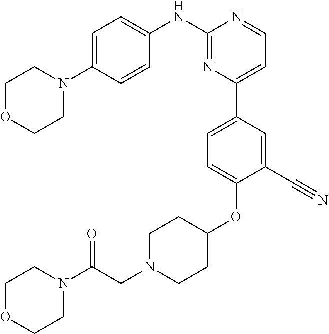 [SMILES: N#Cc1cc(-c2ccnc(Nc3ccc(N4CCOCC4)cc3)n2)ccc1OC1CCN(CC(=O)N2CCOCC2)CC1] | 2-({1-[2-(morpholin- 4-yl)-2- oxoethyl]piperidin-4- yl}oxy)-5-(2-{[4- (morpholin-4- yl)phenyl]amino} pyrimidin-4- yl)benzonitrile | ¹H NMR (CDCl₃) δ 8.43 (d, 1H), 8.30 (d, 1H), 8.23-8.20 (m, 1H), 7.56-7.53 (m, 2H), 7.11-6.95 (m, 5H), 4.62-4.56 (m, 1H), 3.90-3.87 (m, 4H), 3.73-3.60 (m, 8H), 3.24 (s, 2H), 3.16-3.13 (m, 4H), 2.82-2.76 (m, 2H), 2.58-2.48 (m, 2H), 2.12- 1.92 (m, 4H). LC-MS [M + H]⁺ 584.2820 |
| 235 | 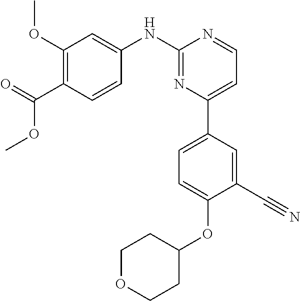 [SMILES: COC(=O)c1ccc(Nc2nccc(-c3ccc(OC4CCOCC4)c(C#N)c3)n2)cc1OC] | methyl 4-({4-[3- cyano-4-(tetrahydro- 2H-pyran-4- yloxy)phenyl] pyrimidin-2-yl} amino)-2- methoxybenzoate | 1H NM (DMSO-d₆) δ 10.1 (br s, 1H), 8.62 (m, 2H), 8.48 (d, 1H), 7.90 (s, 1H), 7.72 (d, 1H), 7.59 (m, 2H), 7.39 (d, 1H), 4.96 (m, 1H), 3.88 (m, 5H), 3.76 (s, 3H), 3.58 (m, 2H), 2.02 (m, 2H), 1.69 (m, 2H); LC-MS [M + H]⁺ 461.1820 |
| 236 | 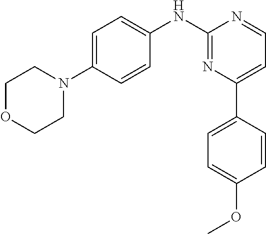 [SMILES: COc1ccc(-c2ccnc(Nc3ccc(N4CCOCC4)cc3)n2)cc1] | 4-(4- methoxyphenyl)-N- [4-(morpholin-4- yl)phenyl]pyrimidin- 2-amine | ¹H NMR (DMSO-d₆) δ 9.38 (s, 1H), 8.42 (s, 1H), 8.13 (d, 2H), 7.66 (d, 2H), 7.27 (d, 1H), 7.08 (d, 2H), 6.92 (d, 2H), 3.84 (s, 3H), 3.74 (t, 4H), 3.04 (t, 4H); LC-MS [M + H]⁺ 363.20 |
| 237 | 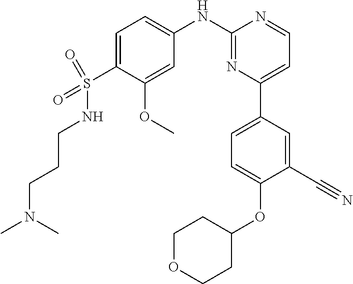 [SMILES: COc1cc(Nc2nccc(-c3ccc(OC4CCOCC4)c(C#N)c3)n2)ccc1S(=O)(=O)NCCCN(C)C] | 4-({4-[3-cyano-4- (tetrahydro-2H- pyran-4- yloxy)phenyl] pyrimidin-2-yl} amino)-N-[3- (dimethylamino) propyl]-2- methoxybenzene- sulfonamide | ¹H NMR (CDCl₃) δ 8.54 (d, 1H); 8.39 (d, 1H); 8.22 (d, 1H); 7.93 (s, 1H); 7.83 (d, 1H); 7.18 (d, 1H); 7.12-7.06 (m, 2H); 4.80-4.75 (m, 1H); 4.07-4.01 (m, 5H); 3.70-3.65 (m, 2H); 3.49 (s, 6H); 2.95 (t, 2H); 2.33 (t, 2H); 2.21 (s, 6H); 2.14-2.08 (m, 2H); 1.98-1.89 (m, 2H). LC- MS [M + H]⁺ 567.2384 |
| 238 | 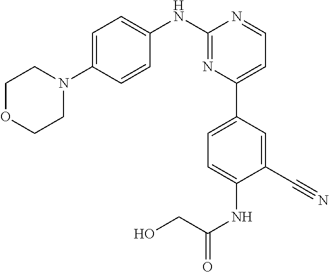 [SMILES: N#Cc1cc(-c2ccnc(Nc3ccc(N4CCOCC4)cc3)n2)ccc1NC(=O)CO] | N-[2-Cyano-4-(2- {[4-(morpholin-4- yl)phenyl]amino} pyrimidin-4-yl)phenyl]- 2-hydroxyacetamide | ¹H NMR (CDCl₃) δ 8.56 (d, 1H), 8.21 (s, 1H), 8.19 (d, 1H), 8.16- 8.13 (m, 1H), 7.51-7.48 (m, 2H), 7.33 (d, 1H), 6.91-6.88 (m, 2H), 6.86 (d, 1H), 5.09 (s, 2H), 4.82 (s, 2H), 3.87-3.85 (m, 4H), 3.14-3.11 (m, 4H). LC-MS [M + H]⁺ 431.1877 |
| 239 | 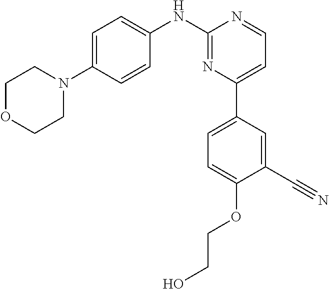 [SMILES: N#Cc1cc(-c2ccnc(Nc3ccc(N4CCOCC4)cc3)n2)ccc1OCCO] | 2-(2- Hydroxyethoxy)-5- (2-{[4-(morpholin-4- yl)phenyl]amino} pyrimidin-4- yl)benzonitrile | ¹H NMR (CDCl₃) δ 8.44 (d, 1H), 8.32 (d, 1H), 8.26-8.23 (d, 1H), 7.56-7.52 (m, 2H), 7.19 (d, 1H), 7.08 (s, 1H), 7.02 (d, 1H), 6.98- 6.95 (m, 2H), 4.28 (t, 2H), 4.08 (br s, 1H), 3.90-3.87 (m, 4H), 3.16- 3.13 (m, 4H). LC-MS [M + H]⁺ 418.1921 |
| 240 | 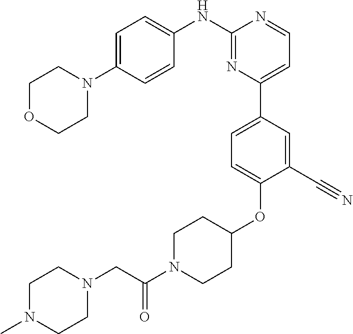 [SMILES: CN1CCN(CC(=O)N2CCC(Oc3ccc(-c4ccnc(Nc5ccc(N6CCOCC6)cc5)n4)cc3C#N)CC2)CC1] | 2-({1-[(4- methylpiperazin-1- yl)acetyl]piperidin-4- yl}oxy)-5-(2-{[4- (morpholin-4- yl)phenyl]amino} pyrimidin-4- yl)benzonitrile | ¹H NMR (CDCl₃) δ 8.34-8.30 (m, 2H), 8.26 (d, 1H), 7.72-7.69 (m, 2H), 7.31-7.16 (m, 3H), 4.96-4.92 (m, 2H), 4.10-4.04 (m, 1H), 4.01- 3.99 (m, 4H), 3.92-3.75 (m, 2H), 3.71-3.65 (m, 1H), 3.62-3.42 (m, 9H), 3.36-3.33 (m, 4H), 2.91 (s, 3H), 2.07-1.96 (m, 4H). LC-MS [M + H]⁺ 597.3330 |
| 241 | 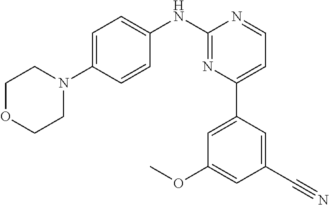 [SMILES: COc1cc(C#N)cc(-c2ccnc(Nc3ccc(N4CCOCC4)cc3)n2)c1] | 3-Methoxy-5-(2-{[4- (morpholin-4- yl)phenyl]amino} pyrimidin-4- yl)benzonitrile | ¹H NMR (CDCl₃) δ 8.35 (d, 1H), 7.91-7.90 (m, 1H), 7.85-7.84 (m, 1H), 7.64-7.61 (m, 2H), 7.33-7.32 (m, 1H), 7.16 (d, 1H), 7.04 (d, 2H), 3.94-3.91 (m, 7H), 3.23-3.21 (m, 4H). LC-MS [M + H]⁺ 388.1760 |
| 242 | 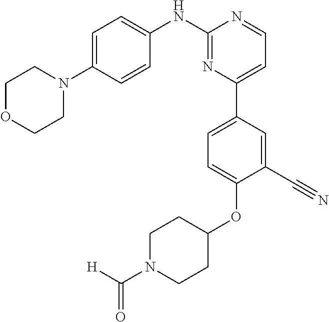 [SMILES: N#Cc1cc(-c2ccnc(Nc3ccc(N4CCOCC4)cc3)n2)ccc1OC1CCN(C=O)CC1] | 2-[(1- formylpiperidin-4- yl)oxy]-5-(2-{[4- (morpholin-4- yl)phenyl]amino} pyrimidin-4- yl)benzonitrile | ¹H NMR (DMSO-d₆) δ 9.67 (s, 1H), 8.55-8.47 (m, 2H), 8.47-8.44 (m, 1H), 8.04 (s, 1H), 7.73 (d, 1H), 7.57 (d, 1H), 7.46 (br s, 1H), 7.15 (br s, 2H), 5.06-5.00 (m, 1H), 3.82 (br s, 4H), 3.68-3.56 (m, 2H), 3.43- 3.37 (m, 2H), 3.22 (br s, 4H), 2.05- 1.93 (m, 2H), 1.79-1.62 (m, 2H). LC-MS [M + H]⁺ 485.2291 |
| 243 | 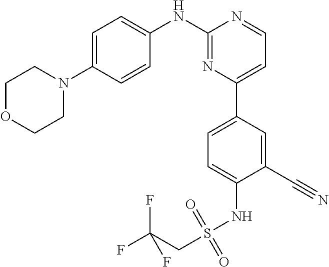 [SMILES: N#Cc1cc(-c2ccnc(Nc3ccc(N4CCOCC4)cc3)n2)ccc1NS(=O)(=O)CC(F)(F)F] | N-[2-cyano-4-(2-{[4- (morpholin-4- yl)phenyl]amino} pyrimidin-4-yl)phenyl]- 2,2,2- trifluoroethane- sulfonamide | ¹H NMR (DMSO-d₆) δ 9.54 (s, 1H), 8.56-8.52 (m, 2H), 8.43-8.41 (m, 1H), 7.70 (d, 1H), 7.65 (d, 2H), 7.43 (d, 1H), 6.95 (d, 2H), 4.66- 4.54 (m, 1H), 3.76-3.73 (m, 6H), 3.08-3.05 (m, 4H). LC-MS [M + H]⁺ 519.1628 |
| 244 | 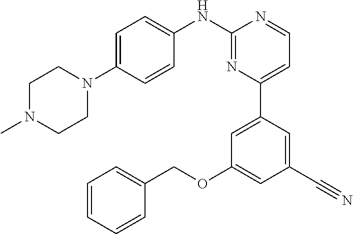 [SMILES: CN1CCN(c2ccc(Nc3nccc(-c4cc(C#N)cc(OCc5ccccc5)c4)n3)cc2)CC1] | 3-(Benzyloxy)-5-(2- {[4-(4- methylpiperazin-1- yl)phenyl]amino} pyrimidin-4- yl)benzonitrile | LC-MS [M + H]⁺ 477.2447 |
| 245 | 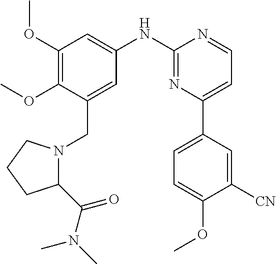 [SMILES: COc1ccc(-c2ccnc(Nc3cc(CN4CCCC4C(=O)N(C)C)c(OC)c(OC)c3)n2)cc1C#N] | 1-(5-{[4-(3-Cyano-4- methoxyphenyl) pyrimidin-2-yl]amino}- 2,3- dimethoxybenzyl)- N,N- dimethylprolinamide | ¹H NMR (MeOH-d₄) δ 8.44-8.33 (m, 3H), 7.64 (d, 1H), 7.26-7.17 (m, 3H), 4.01 (s, 3H), 3.96-3.82 (m, 2H), 3.91 (s, 3H), 3.78 (s, 3H), 3.75-3.71 (m, 1H), 3.18 (bs, 1H), 3.01 (s, 3H), 2.89 (s, 3H), 2.67- 2.63 (m, 1H), 2.26-2.19 (m, 1H), 1.88-1.82 (m, 2H), 1.74-1.69 (m, 1H). LC-MS [M + H]⁺ 517.2563 |
| 246 | 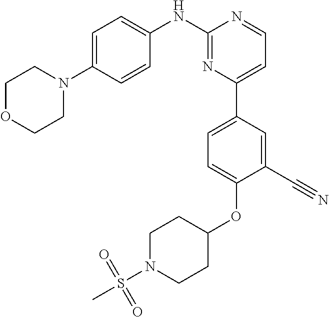 [SMILES: CS(=O)(=O)N1CCC(Oc2ccc(-c3ccnc(Nc4ccc(N5CCOCC5)cc4)n3)cc2C#N)CC1] | 2-{[1- (Methylsulfonyl) piperidin-4-yl]oxy}- 5-(2- {[4-(morpholin-4- yl)phenyl]amino} pyrimidin-4- yl)benzonitrile | ¹H NMR (CDCl₃) δ 8.45 (d, 1H), 8.32 (d, 1H), 8.26-8.23 (m, 1H), 7.56-7.52 (m, 2H), 7.17 (s, 1H), 7.70 (d, 1H), 7.02 (d, 1H), 6.98- 6.95 (m, 2H), 4.89-4.84 (m, 1H), 3.90-3.87 (m, 4H), 3.62-3.57 (m, 2H), 3.32-3.23 (m, 2H), 3.16-3.13 (m, 4H), 2.85 (s, 3H), 2.12-2.05 (m, 4H). LC-MS [M + H]⁺ 535.2219 |
| 247 | 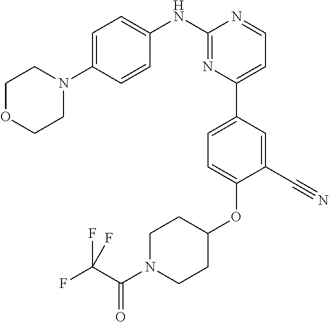 [SMILES: N#Cc1cc(-c2ccnc(Nc3ccc(N4CCOCC4)cc3)n2)ccc1OC1CCN(C(=O)C(F)(F)F)CC1] | 5-(2-{[4-(Morpholin- 4- yl)phenyl]amino} pyrimidin-4-yl)-2-{[1- (trifluoroacetyl) piperidin-4- yl]oxy}benzonitrile | ¹H NMR (CDCl₃) δ 8.45 (d, 1H), 8.33 (d, 1H), 8.26-8.23 (m, 1H), 7.55-7.53 (m, 2H), 7.21 (s, 1H), 7.08 (d, 1H), 7.02 (d, 1H), 6.97- 6.94 (m, 2H), 4.92-4.88 (m, 1H), 4.18-4.11 (m, 1H), 3.90-3.80 (m, 6H), 3.63-3.56 (m, 1H), 3.16-3.13 (m, 4H), 2.13-1.96 (m, 4H). LC- MS [M + H]⁺ 553.2073 |
| 248 | 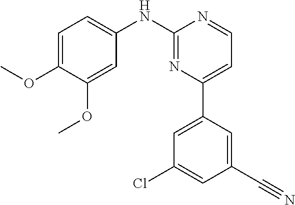 [SMILES: COc1ccc(Nc2nccc(-c3cc(Cl)cc(C#N)c3)n2)cc1OC] | 3-Chloro-5-{2-[(3,4- dimethoxyphenyl) amino]pyrimidin-4- yl}benzonitrile | ¹H NMR (DMSO-d₆) δ 9.66 (s, 1H), 8.62-8.55 (m, 3H), 8.24-8.23 (m, 1H), 7.70 (s, 1H), 7.55 (d, 1H), 7.15 (d, 1H), 6.91 (d, 1H), 3.82 (s, 3H), 3.73 (s, 3H). LC-MS [M + H]⁺ 367.0839 |
| 249 | 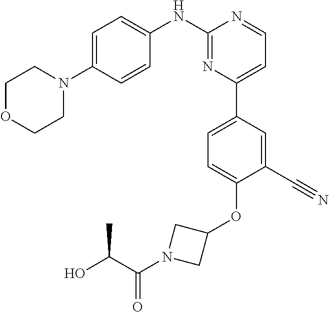 [SMILES: C[C@H](O)C(=O)N1CC(Oc2ccc(-c3ccnc(Nc4ccc(N5CCOCC5)cc4)n3)cc2C#N)C1] | 2-({1-[(2S)-2- hydroxypropanoyl] azetidin-3-yl}oxy)-5- (2-{[4-(morpholin-4- yl)phenyl]amino} pyrimidin-4- yl)benzonitrile | ¹H NMR (DMSO-d₆) δ 6.59 (s, 1H), 8.57 (d, 1H), 8.52 (d, 1H), 8.46- 8.43 (m, 1H), 7.68 (d, 2H), 7.44 (d, 1H), 7.19 (d, 1H), 7.05-7.03 (m, 2H), 5.33-5.29 (m, 1H), 4.85-4.77 (m, 1H), 4.46-4.31 (m, 2H), 4.18- 4.14 (m, 1H), 3.92-3.88 (m, 1H), 3.79-3.77 (m, 4H), 3.14 (br s, 4H). LC-MS [M + H]⁺ 501.2123 |
| 250 | AND Enantiomer 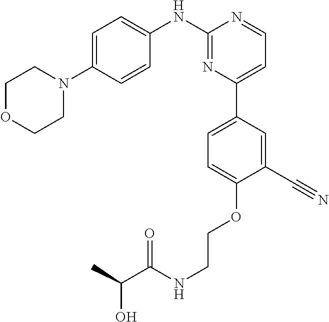 [SMILES: C[C@H](O)C(=O)NCCOc1ccc(-c2ccnc(Nc3ccc(N4CCOCC4)cc3)n2)cc1C#N] | (2S)-N-{2-[2-cyano- 4-(2-{[4-(morpholin- 4- yl)phenyl]amino} pyrimidin-4- yl)phenoxy]ethyl}-2- hydroxypropanamide | ¹H NMR (DMSO-d₆) δ 9.53 (s, 1H), 8.52-8.44 (m, 3H), 7.96 (t, 1H), 7.66 (d, 2H), 7.47 (d, 1H), 7.42 (d, 1H), 7.26 (s, 1H), 7.14 (s, 1H), 7.01-6.98 (m, 2H), 4.30 (t, 2H), 4.02-3.96 (m, 1H), 3.78-3.75 (m, 4H), 3.56-3.50 (m, 2H), 3.13-3.10 (m, 4H). LC-MS [M + H]⁺ 489.2306 |
| 251 | 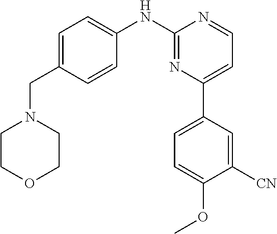 [SMILES: COc1ccc(-c2ccnc(Nc3ccc(CN4CCOCC4)cc3)n2)cc1C#N] | 2-Methoxy-5-(2-{[4- (morpholin-4- ylmethyl)phenyl] amino}pyrimidin-4- yl)benzonitrile | ¹H NMR (CDCl₃) δ 8.46 (d, 1H), 8.31-8.26 (m, 2H), 7.64-7.61 (m, 2H), 7.35-7.32 (m, 3H), 7.26-7.06 (m, 2H), 4.02 (s, 3H), 3.73-3.71 (m, 4H), 3.48 (s, 2H), 2.50-2.45 (m, 4H). LC-MS [M + H]⁺ 402.1840 |
| 252 | 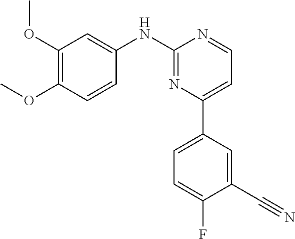 [SMILES: COc1ccc(Nc2nccc(-c3ccc(F)c(C#N)c3)n2)cc1OC] | 5-{2-[(3,4- Dimethoxyphenyl) amino]pyrimidin-4-yl}- 2-fluorobenzonitrile | ¹H NMR (CDCl₃) δ 8.53 (d, 1H), 7.99-7.91 (m, 2H), 7.77-7.73 (m, 1H), 7.42 (s, 1H), 7.39 (d, 1H), 7.12 (d, 1H), 7.08-7.05 (m, 1H), 6.89 (d, 1H), 3.93 (s, 3H), 3.90 (s, 3H). LC-MS [M + H]⁺ 351.1320 |
| 253 | 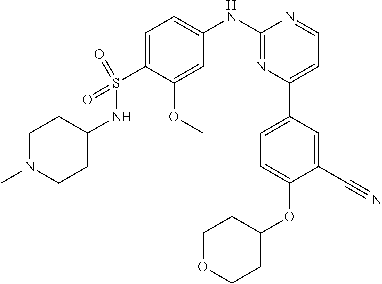 [SMILES: COc1cc(Nc2nccc(-c3ccc(OC4CCOCC4)c(C#N)c3)n2)ccc1S(=O)(=O)NC1CCN(C)CC1] | 4-({4-[3-cyano-4- (tetrahydro-2H- pyran-4- yloxy)phenyl] pyrimidin-2-yl} amino)-2- methoxy-N-(1- methylpiperidin-4- yl)benzenesulfonamide | ¹H NMR (DMSO-d₆) δ 10.21 (s, 1H); 8.65 (d, 1H); 8.61 (s, 1H), 8.47 (d, 1H); 8.02 (s, 1H); 7.69- 7.56 (m, 3H), 7.49-7.38 (m, 2H); 4.99-4.96 (m, 1H); 3.97 (s, 3H); 3.90-3.85 (m, 2H); 3.59-3.54 (m, 2H); 3.32 (m, 2H); 3.21-3.19 (m, 1H); 2.93 (q, 1H); 2.73 (d, 1H); 2.65 (d, 2H); 2.07-2.04 (m, 2H); 1.80-1.60 (m, 6H). LC-MS [M + H]⁺ 579.2220 |
| 254 | 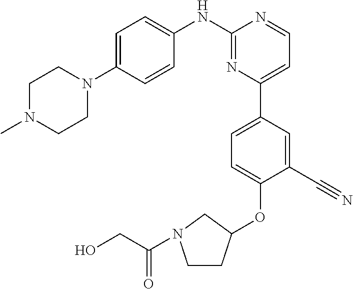 [SMILES: CN1CCN(c2ccc(Nc3nccc(-c4ccc(OC5CCN(C(=O)CO)C5)c(C#N)c4)n3)cc2)CC1] | 2-{[1- (hydroxyacetyl) pyrrolidin-3-yl] oxy}-5-(2-{[4-(4- methylpiperazin-1- yl)phenyl]amino} pyrimidin-4- yl)benzonitrile | ¹H NMR (MeOH-d₄) δ 8.50-8.44 (m, 2H), 8.40 (d, 1H), 7.60-7.56 (m, 2H), 7.40 (dd, 1H), 7.23 (d, 1H), 7.01-6.99 (m, 2H,) 4.23 (s, 2H), 4.20-4.16 (m, 1H), 3.90-3.60 (m, 6H), 3.20-3.17 (m, 2H), 2.70- 2.63 (m, 6H), 2.40 (s, 3H). LC-MS [M + H]⁺ 514.2490 |
| 255 | 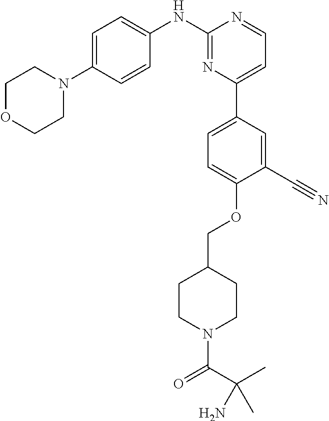 [SMILES: CC(C)(N)C(=O)N1CCC(COc2ccc(-c3ccnc(Nc4ccc(N5CCOCC5)cc4)n3)cc2C#N)CC1] | 2-{[1-(2- methylalanyl) piperidin-4-yl] methoxy}-5- (2-{[4-(morpholin-4- yl)phenyl]amino} pyrimidin-4- yl)benzonitrile | ¹H NMR (DMSO-d₆) δ 9.53 (s, 1H), 8.52-8.45 (m, 3H), 8.13 (br s, 3H), 7.66 (d, 2H), 7.45-7.40 (m, 2H), 6.98 (d, 2H), 4.30 (br s, 1H), 4.16 (d, 2H), 3.78-3.75 (m, 4H), 3.11- 3.08 (m, 4H), 2.23-2.14 (m, 1H), 1.92-1.86 (m, 2H), 1.56 (s, 6H), 1.32-1.22 (m, 2H). LC-MS [M + H]⁺ 556.3013 |
| 256 | 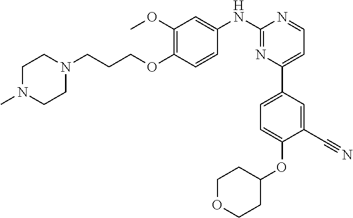 [SMILES: COc1cc(Nc2nccc(-c3ccc(OC4CCOCC4)c(C#N)c3)n2)ccc1OCCCN1CCN(C)CC1] | 5-[2-({3-methoxy-4- [3-(4- methylpiperazin-1- yl)propoxy]phenyl} amino)pyrimidin-4- yl]-2-(tetrahydro-2H- pyran-4- yloxy)benzonitrile | ¹H NMR (DMSO-d₆) δ 9.59 (s, 1H), 8.56 (d, 1H), 8.53 (d, 1H), 8.43 (dd, 1H), 7.70 (br s, 1H), 7.54 (d, 1H), 7.44 (d, 1H), 7.21 (d, 1H), 7.94 (d, 1H), 4.95 (sept, 1H), 4.00 (t, 2H), 3.93-3.83 (m, 2H), 3.82 (s, 3H), 3.56 (ddd, 4H), 3.17 (s, 3H), 3.00-3.20 (br s, 2H), 2.83 (br s, 4H), 2.10-2.00 (m, 4H), 1.74-1.62 (m, 2H); LC-MS [M + H]⁺ 559.3018 |
| 257 | 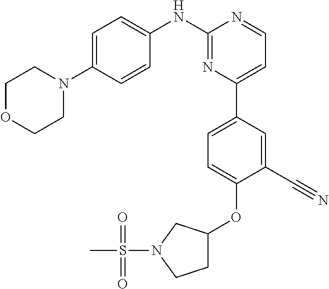 [SMILES: CS(=O)(=O)N1CCC(Oc2ccc(-c3ccnc(Nc4ccc(N5CCOCC5)cc4)n3)cc2C#N)C1] | 2-{[1- (methylsulfonyl) pyrreolidin-3-yl] oxy}-5- (2-{[4-(morpholin-4- yl)phenyl]amino} pyrimidin-4- yl)benzonitrile | LC-MS [M + H]⁺ 521.1969 |
| 258 | 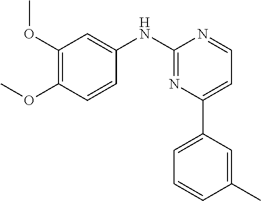 | N-(3,4- Dimethoxyphenyl)-4- (3- methylphenyl) pyrimidin-2-amine | ¹H NMR (CDCl₃) δ 8.43 (d, 1H), 7.91 (s, 1H), 7.85 (d, 1H), 7.63 (d, 1H), 7.39-7.29 (m, 3H), 7.12 (d, 1H), 7.01-6.98 (m, 1H), 6.86 (d, 1H), 3.94 (s, 3H), 3.89 (s, 3H), 2.43 (s, 3H). LC-MS [M + H]⁺ 322.1639 |
| 259 | 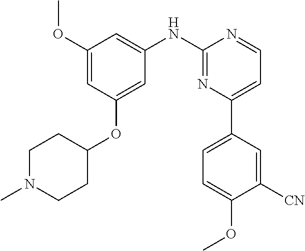 [SMILES: COc1cc(Nc2nccc(-c3ccc(OC)c(C#N)c3)n2)cc(OC2CCN(C)CC2)c1] | 2-Methoxy-5-[2-({3- methoxy-5-[(1- methylpiperidin-4- yl)oxy]phenyl}amino) pyrimidin-4- yl]benzonitrile | ¹H NMR (CDCl₃) δ 8.47 (d, 1H), 8.35 (d, 1H), 8.28-8.25 (m, 1H), 7.25 (bs, 1H), 7.09-7.07 (m, 2H), 7.01 (s, 1H), 6.89 (s, 1H), 6.22- 6.21 (m, 1H), 6.22-6.21 (m, 1H), 4.35 (bs, 1H), 4.02 (s, 3H), 3.84 (s, 3H), 2.78-2.70 (m, 2H), 2.37-2.34 (m, 2H), 2.34 (s, 3H), 2.08-2.02 (m, 2H) 1.93-1.88 (m, 2H). LC-MS [M + H]⁺ 446.2199 |
| 260 | 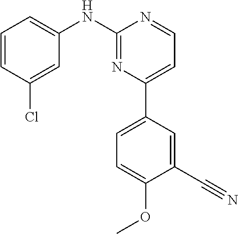 [SMILES: COc1ccc(-c2ccnc(Nc3cccc(Cl)c3)n2)cc1C#N] | 5-{2-[(3- Chlorophenyl)amino] pyrimidin-4-yl}-2- methoxybenzonitrile | ¹H NMR (CDCl₃) δ 8.50 (d, 1H), 8.31-8.28 (m, 2H), 7.95 (t, 1H), 7.43-7.40 (m, 1H), 7.30-7.25 (m, 3H), 7.13-7.09 (m, 2H), 7.05-7.02 (m, 1H), 4.02 (s, 3H). LC-MS [M + H]⁺ 337.0828 |
| 261 | 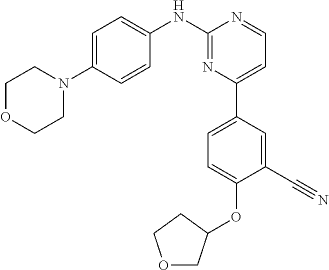 [SMILES: N#Cc1cc(-c2ccnc(Nc3ccc(N4CCOCC4)cc3)n2)ccc1OC1CCOC1] | 5-(2-{[4-(Morpholin- 4- yl)phenyl]amino} pyrimidin-4-yl)-2- (tetrahydrofuran-3- yloxy)benzonitrile | ¹H NMR (CDCl₃) δ 8.44 (d, 1H), 8.32 (d, 1H), 8.25-8.21 (m, 1H), 7.56-7.53 (m, 2H), 7.07 (s, 1H), 7.03-6.95 (m, 4H), 5.11-5.07 (m, 1H), 4.16-4.12 (m, 1H), 4.07-4.03 (m, 2H), 4.00-3.95 (m, 1H), 3.90- 3.87 (m, 4H), 3.16-3.14 (m, 4H), 2.32-2.26 (m, 2H). LC-MS [M + H]⁺ 444.2101 |
| 262 | 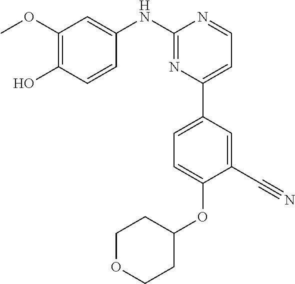 [SMILES: COc1cc(Nc2nccc(-c3ccc(OC4CCOCC4)c(C#N)c3)n2)ccc1O] | 5-{2-[(4-hydroxy-3- methoxyphenyl) amino]pyrimidin- 4-yl}-2- (tetrahydro-2H- pyran-4- yloxy)benzonitrile | ¹H NMR (DMSO-d₆) δ 9.42 (s, 1H), 8.62 (s, 1H), 8.55 (d, 1H), 8.49 (d, 1H), 8.43 (dd, 1H), 7.56 (br s, 1H), 7.54 (d, 1H), 7.39 (d, 1H), 7.05 (d, 1H), 6.71 (d, 1H), 4.95 (sept, 1H), 3.92-3.83 (m, 2H), 3.81 (s, 3H), 3.55 (ddd, 2H), 2.10-2.00 (m, 2H), 1.63-1.75 (m, 2H); LC-MS [M + H]⁺ 419.1718 |
| 263 | 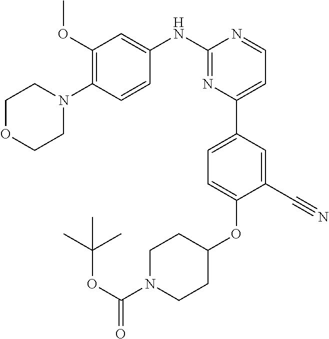 [SMILES: COc1cc(Nc2nccc(-c3ccc(OC4CCN(C(=O)OC(C)(C)C)CC4)c(C#N)c3)n2)ccc1N1CCOCC1] | tert-butyl 4-[2- cyano-4-(2-{[3- methoxy-4- (morpholin-4- yl)phenyl]amino} pyrimidin-4- yl)phenoxy]piperidine- 1-carboxylate | LC-MS [M + H]⁺ 587.30 |
| 264 | 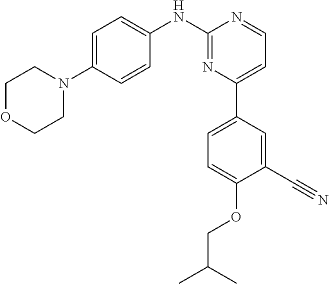 [SMILES: CC(C)COc1ccc(-c2ccnc(Nc3ccc(N4CCOCC4)cc3)n2)cc1C#N] | 2-(2- Methylpropoxy)-5- (2-{[4-(morpholin-4- yl)phenyl]amino} pyrimidin-4- yl)benzonitrile | ¹H NMR (CDCl₃) δ 8.43 (d, 1H), 8.30 (d, 1H), 8.23-8.21 (m, 1H), 7.56-7.53 (m, 2H), 7.08 (s, 1H), 7.04 (d, 1H), 7.02 (d, 1H), 6.97- 6.94 (m, 2H), 3.92-3.87 (m, 6H), 3.16-3.13 (m, 4H), 2.25-2.18 (m, 1H), 1.10 (d, 6H). LC-MS [M + H]⁺ 430.2299 |
| 265 | 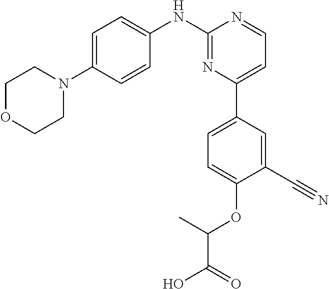 [SMILES: CC(Oc1ccc(-c2ccnc(Nc3ccc(N4CCOCC4)cc3)n2)cc1C#N)C(=O)O] | 2-[2-Cyano-4-(2-{[4- (morpholin-4- yl)phenyl]amino} pyrimidin-4- yl)phenoxy]propanoic acid | ¹H NMR (DMSO-d₆) δ 9.43 (s, 1H), 8.48-8.46 (m, 2H), 8.35-8.32 (m, 1H), 7.63 (d, 2H), 7.35 (d, 1H), 7.13 (d, 1H), 6.92 (d, 2H), 4.77- 4.74 (m, 1H), 3.75-3.73 (m, 4H), 3.06-3.03 (m, 4H), 1.55 (d, 3H). LC-MS [M + H]⁺ 446.1880 |
| 266 | 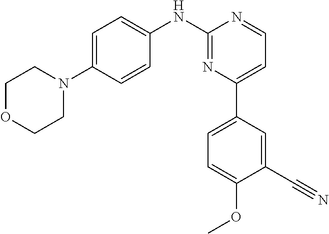 [SMILES: COc1ccc(-c2ccnc(Nc3ccc(N4CCOCC4)cc3)n2)cc1C#N] | 2-Methoxy-5-(2-{[4- (morpholin-4- yl)phenyl]amino} pyrimidin-4- yl)benzonitrile | LC-MS [M + H]⁺ 388.1770 |
| 267 | 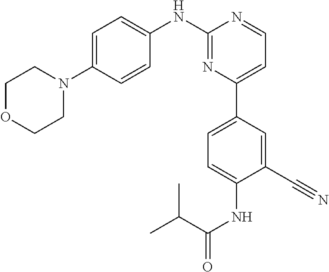 [SMILES: CC(C)C(=O)Nc1ccc(-c2ccnc(Nc3ccc(N4CCOCC4)cc3)n2)cc1C#N] | N-[2-Cyano-4-(2- {[4-(morpholin-4- yl)phenyl]amino} pyrimidin-4-yl)phenyl]- 2- methylpropanamide | ¹H NMR (DMSO-d₆) δ 9.59 (br s, 1H), 8.91 (d, 1H), 8.54-8.50 (m, 2H), 7.75 (d, 1H), 7.72-7.68 (m, 2H), 7.46 (d, 1H), 7.02-6.98 (m, 2H), 3.80-3.75 (m, 4H), 3.14-3.06 (m, 4H), 2.96-2.89 (m, 1H), 1.29 (d, 6H). LC-MS [M + H]⁺ 443.2147 |
| 268 | 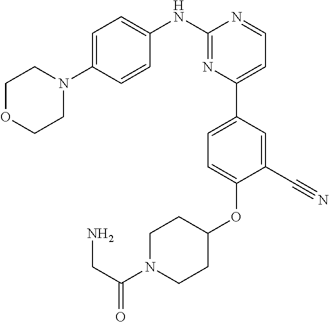 [SMILES: N#Cc1cc(-c2ccnc(Nc3ccc(N4CCOCC4)cc3)n2)ccc1OC1CCN(C(=O)CN)CC1] | 2-[(1- Glycylpiperidin-4- yl)oxy]-5-(2-{[4- (morpholin-4- yl)phenyl]amino} pyrimidin-4- yl)benzonitrile | LC-MS [M + H]⁺ 514.2535 |
| 269 | 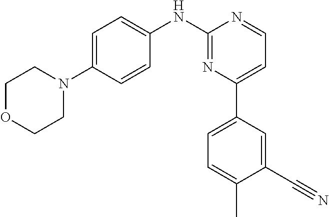 [SMILES: Cc1ccc(-c2ccnc(Nc3ccc(N4CCOCC4)cc3)n2)cc1C#N] | 2-Methyl-5-(2-{[4- (morpholin-4- yl)phenyl]amino} pyrimidin-4- yl)benzonitrile | ¹H NMR (DMSO-d₆) δ 9.50 (s, 1H), 8.53-8.51 (m, 2H), 8.38-8.35 (m, 1H), 7.65-7.63 (m, 3H), 7.42 (d, 1H), 6.93 (d, 2H), 3.76-3.73 (m, 4H), 3.06-3.03 (m, 4H), 2.56 (s, 3H). LC-MS [M + H]⁺ 372.1770 |
| 270 | 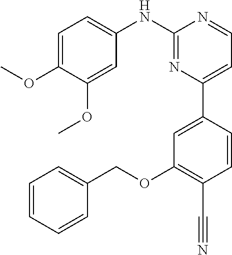 [SMILES: COc1ccc(Nc2nccc(-c3ccc(C#N)c(OCc4ccccc4)c3)n2)cc1OC] | 2-(Benzyloxy)-4-{2- [(3,4- dimethoxyphenyl) amino]pyrimidin-4- yl}benzonitrile | ¹H NMR (CDCl₃) δ 8.50 (d, 1H), 7.79 (d, 1H), 7.70-7.64 (m, 2H), 7.51-7.26 (m, 6H), 7.15 (br s, 1H), 7.14-7.11 (m, 1), 7.07 (d, 1H), 6.88 (d, 1H), 5.31 (s, 2H), 3.91 (s, 3H), 3.89 (s, 3H). C-MS [M + H]⁺ 439.1744 |
| 271 | 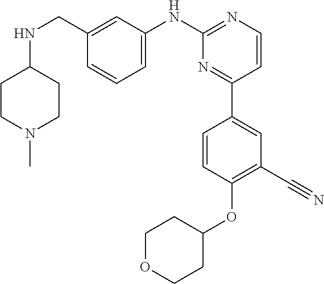 [SMILES: CN1CCC(NCc2cccc(Nc3nccc(-c4ccc(OC5CCOCC5)c(C#N)c4)n3)c2)CC1] | 5-{2-[(3-{[(1- Methylpiperidin-4- yl)amino]methyl} phenyl)amino] pyrimidin- 4-yl}-2-(tetrahydro- 2H-pyran-4- yloxy)benzonitrile | ¹H NMR (MeOH d-4) δ 8.57 (s, 1H), 8.50 (br s, 1H), 8.38 (d, 1H), 8.15 (br s, 1H), 7.67 (d, 1H), 7.46- 7.36 (m, 3H), 7.17 (d, 1H), 4.96 (m, 3H), 4.32 (s, 6H), 3.14 (t, 2H), 2.90 (s, 3H), 2.49 (d, 2H), 2.14- 2.00 (m, 5H), 1.88-1.80 (m, 2H). LC-MS [M + H]⁺ 499.2720 |
| 272 | 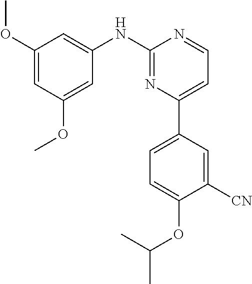 [SMILES: COc1cc(Nc2nccc(-c3ccc(OC(C)C)c(C#N)c3)n2)cc(OC)c1] | 5-{2-[(3,5- Dimethoxyphenyl) amino]pyrimidin- 4-yl}-2-(propan-2- yloxy)benzonitrile | ¹H NMR (CDCl₃) δ 9.64 (s, 1H), 8.54-8.52 (m, 2H), 8.43-8.39 (m, 1H), 7.45-7.44 (m, 2H), 7.13-7.12 (d, 2H), 6.12 (s, 1H), 4.94-4.86 (m, 1H), 3.73 (s, 6H), 1.35-1.33 (d, 6H). LC-MS [M + H]⁺ 391.3 |
| 273 | 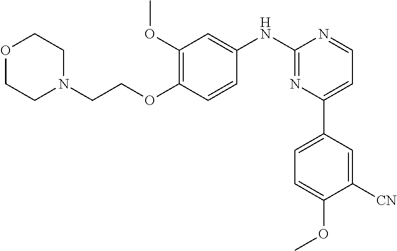 [SMILES: COc1ccc(-c2ccnc(Nc3ccc(OCCN4CCOCC4)c(OC)c3)n2)cc1C#N] | 2-Methoxy-5-[2-({3- methoxy-4-[2- (morpholin-4- yl)ethoxy]phenyl} amino)pyrimidin-4- yl]benzonitrile | ¹H NMR (CDCl₃—) δ 8.45 (d, 1H), 8.35 (d, 1H), 8.25-8.23 (m, 1H), 7.49 (d, 1H), 7.21 (s, 1H), 7.08- 7.00 (m, 3H), 6.92 (d, 1H), 4.18- 4.15 (m, 2H), 4.01 (s, 3H), 3.92 (s, 3H), 3.76-3.74 (m, 4H), 2.86-2.83 (m, 2H), 2.62-2.59 (m, 4H). LC- MS [M + H]⁺ 462.2143 |
| 274 | 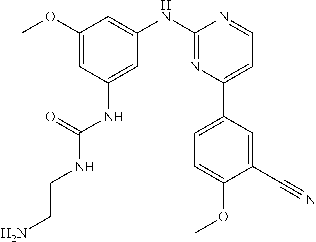 [SMILES: COc1cc(NC(=O)NCCN)cc(Nc2nccc(-c3ccc(OC)c(C#N)c3)n2)c1] | 1-(2-Aminoethyl)-3- (2-{[4-(3-cyano-4- methoxyphenyl) pyrimidin-2-yl] amino}-5- methoxyphenyl)urea | ¹H NMR (DMSO-d₆) δ 9.61 (s, 1H), 8.62-8.52 (m, 4H), 7.47 (d, 1H), 7.45-7.38 (m, 2H), 7.10 (s, 1H), 6.80 (s, 1H), 6.26 (s, 1H), 4.02 (s, 3H), 3.73 (s, 3H), 3.14-3.04 (m, 2H), 2.66-2.57 (m, 2H); LC-MS [M + H]⁺ 434.1939. |
| 275 | 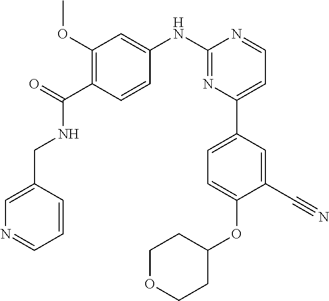 [SMILES: COc1cc(Nc2nccc(-c3ccc(OC4CCOCC4)c(C#N)c3)n2)ccc1C(=O)NCc1cccnc1] | 4-({4-[3-cyano-4- (tetrahydro-2H- pyran-4- yloxy)phenyl] pyrimidin-2-yl} amino)-2- methoxy-N-(pyridin- 3- ylmethyl)benzamide | ¹H NMR (DMSO-d₆) δ 10.1 (s, 1H), 8.82 (t, 1H), 8.74 (s, 1H), 8.66 (d, 1H) 8.64 (d, 1H), 8.62 (d, 1H), 8.47 (dd, 1H), 8.19 (d, 1H), 7.98 (s, 1H), 7.82 (d, 1H), 7.76 (dd, 1H), 7.59- 7.56 (m, 2H), 7.37 (dd, 1H), 4.98- 4.93 (m, 1H), 4.61 (d, 2H), 4.00 (s, 3H), 3.90-3.85 (m, 2H), 3.59-3.53 (m, 2H), 2.07-2.02 (m, 2H), 1.73- 1.65 (m, 2H). LC-MS [M + H]⁺ 537.2234 |
| 276 | 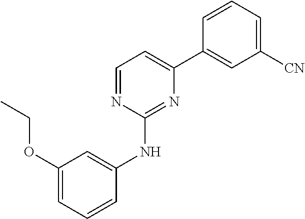 [SMILES: CCOc1cccc(Nc2nccc(-c3cccc(C#N)c3)n2)c1] | 3-{2-[(3- Ethoxyphenyl)amino] pyrimidin-4- yl}benzonitrile | ¹H NMR (CDCl₃) δ 8.54 (d, 1H), 8.41-8.40 (m, 1H), 8.29-8.26 (m, 1H), 7.79-7.77 (m, 1H), 7.64-7.60 (m, 1H), 7.52-7.51 (m, 1H), 7.32- 7.24 (m, 2H), 7.16-7.10 (m, 2H), 6.65-6.62 (m, 2H), 4.12-4.07 (m, 2H), 1.48-1.44 (m, 3H). LC-MS [M + H]⁺ 317.1423 |
| 277 | 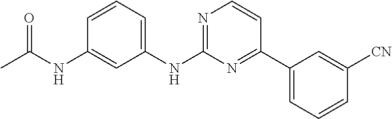 [SMILES: CC(=O)Nc1cccc(Nc2nccc(-c3cccc(C#N)c3)n2)c1] | N-(3-{[4-(3- Cyanophenyl) pyrimidin-2- yl]amino}phenyl) acetamide | ¹H NMR (CD-3OD) δ 8.65 (s, 1H), 8.52 (d, 1H), 8.48-8.44 (m, 2H), 7.87-7.85 (m, 1H), 7.71-7.67 (m, 1H), 7.38 (d, 1H), 7.31-7.22 (m, 2H), 7.10-7.07 (m, 1H), 2.17 (s, 3H). LC-MS [M + H]⁺ 330.1344 |
| 278 | 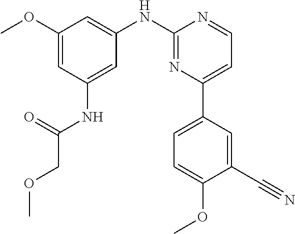 [SMILES: COCC(=O)Nc1cc(Nc2nccc(-c3ccc(OC)c(C#N)c3)n2)cc(OC)c1] | N-(3-{[4-(3-Cyano- 4- methoxyphenyl) pyrimidin-2-yl]amino}- 5-methoxyphenyl)-2- methoxyacetamide | ¹H NMR (DMSO-d₆) δ 9.69 (d, 2H), 8.62 (d, 1H), 8.57-8.53 (m, 2H), 7.94 (s, 1H), 7.49 (d, 1H), 7.41 (d, 1H), 7.19 (s, 1H), 6.90 (s, 1H), 4.02 (s, 2H), 4.01 (s, 3H), 3.75 (s, 3H), 3.39 (s, 3H); LC-MS [M + H]⁺ 420.1676. |
| 279 | 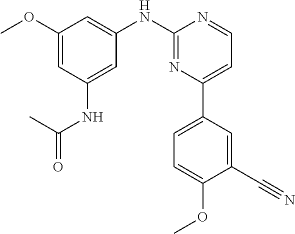 [SMILES: COc1cc(NC(C)=O)cc(Nc2nccc(-c3ccc(OC)c(C#N)c3)n2)c1] | N-(3-{[4-(3-Cyano- 4- methoxyphenyl) pyrimidin-2-yl]amino}- 5- methoxyphenyl) acetamide | ¹H NMR (DMSO-d₆) δ 9.91 (s, 1H), 9.71 (s, 1H), 8.64 (d, 1H), 8.58- 8.47 (m, 2H), 7.82 (s, 1H), 7.49 (d, 1H), 7.41 (d, 1H), 7.16 (s, 1H), 6.84 (s, 1H), 4.02 (s, 3H), 3.74 (s, 3H), 2.05 (s, 3H). LC-MS [M + H]⁺ 390.1565. |
| 280 | 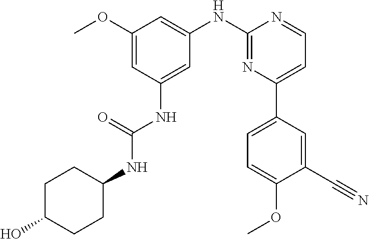 [SMILES: COc1cc(NC(=O)N[C@H]2CC[C@H](O)CC2)cc(Nc2nccc(-c3ccc(OC)c(C#N)c3)n2)c1] | 1-(3-{[4-(3-Cyano-4- methoxyphenyl) pyrimidin-2-yl]amino}- 5-methoxyphenyl)-3- (trans-4- hydroxycyclohexyl) urea | ¹H NMR (DMSO-d₆) δ 9.60 (s, 1H), 8.62-8.55 (m, 2H), 8.54 (d, 1H), 8.28 (s, 1H), 7.66 (br s, 3H), 7.47 (sd, 1H), 7.42-7.36 (m, 2H), 7.09 (s, 1H), 6.79 (s, 1H), 5.99 (d, 1H), 4.55 (br s, 1H), 4.02 (s, 3H), 3.72 (s, 3H), 3.50-3.25 (m, 6H), 1085- 1.65 (m, 4H); LC-MS [M + H]⁺ 489.2245. |
| 281 | 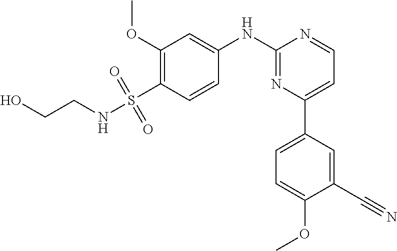 [SMILES: COc1ccc(-c2ccnc(Nc3ccc(S(=O)(=O)NCCO)c(OC)c3)n2)cc1C#N] | 4-{[4-(3-Cyano-4- methoxyphenyl) pyrimidin-2-yl]amino}- N-(2-hydroxyethyl)- 2- methoxybenzene- sulfonamide | ¹H NMR (DMSO-d₆) δ 10.17 (s, 1 H), 8.65 (d, 1 H), 8.61 (d, 1 H), 8.53 (dd, 1 H), 7.99 (br. s., 1 H), 7.64 (d, 1 H), 7.60 (d, 1 H), 7.47 (d, 1 H), 7.40 (dd, 1 H), 6.84 (t, 1 H), 6.56 (s, 1 H), 4.65 (t, 1 H), 4.02 (s, 3 H), 3.95 (s, 3 H), 2.77 (q, 2 H) |
| 282 | 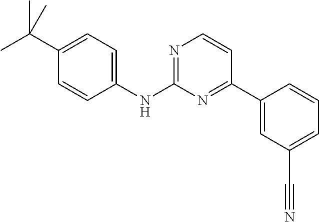 [SMILES: CC(C)(C)c1ccc(Nc2nccc(-c3cccc(C#N)c3)n2)cc1] | 3-{2-[(4-tert- Butylphenyl)amino] pyrimidin-4- yl}benzonitrile | ¹H NMR (CDCl₃) δ 8.52 (d, 1H), 8.38-8.37 (m, 1H), 8.30-8.27 (m, 1H), 7.79-7.76 (m, 1H), 7.64-7.57 (m, 3H), 7.43-7.40 (m, 2H), 7.35 (s, 1H), 7.12 (d, 1H), 1.34 (s, 9H). LC-MS [M + H]⁺ 329.1764 |
| 283 | 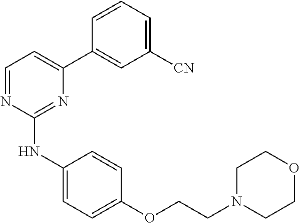 [SMILES: N#Cc1cccc(-c2ccnc(Nc3ccc(OCCN4CCOCC4)cc3)n2)c1] | 3-[2-({4-[2- (Morpholin-4- yl)ethoxy]phenyl} amino)pyrimidin-4- yl]benzonitrile | ¹H NMR (CDCl₃) δ 8.49 (d, 1H), 8.37-8.36 (m, 1H), 8.27-8.24 (m, 1H), 7.78-7.76 (m, 1H), 7.63-7.59 (m, 1H), 7.56-7.53 (m, 2H), 7.17 (s, 1H), 7.10 (d, 1H), 6.96-6.94 (m, 2H), 4.15-4.12 (m, 2H), 3.77-3.75 (m, 4H), 2.84-2.81 (m, 2H), 2.60 (s, 4H). LC-MS [M + H]⁺ 402.1929 |
| 284 | 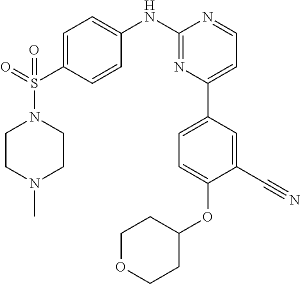 [SMILES: CN1CCN(S(=O)(=O)c2ccc(Nc3nccc(-c4ccc(OC5CCOCC5)c(C#N)c4)n3)cc2)CC1] | 5-[2-({4-[(4- methylpiperazin-1- yl)sulfonyl]phenyl} amino)pyrimidin-4- yl]-2-(tetrahydro-2H- pyran-4- yloxy)benzonitrile | ¹H NMR (DMSO-d₆) δ 10.4 (s, 1H), 9.48 (br s, 1H), 8.66 (d, 1H), 8.59 (d, 1H), 8.50 (dd, 1H), 8.13 (d, 2H), 7.74 (d, 2H), 7.65 (d, 1H), 7.58 (d, 1H), 4.99-4.93 (m, 1H), 3.91-3.85 (m, 2H), 3.82-2.64 (m, 2H), 3.59-3.53 (m, 2H), 3.55-3.33 (m, 4H), 3.25-3.05 (m, 2H), 2.78 (s, 3H), 2.07-2.03 (m, 2H), 1.74-1.66 (m, 2H). LC-MS [M + H]⁺ 535.2119 |
| 285 | 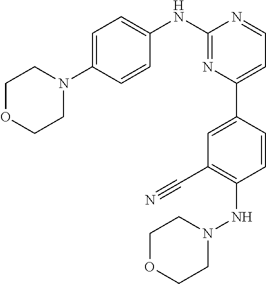 [SMILES: N#Cc1cc(-c2ccnc(Nc3ccc(N4CCOCC4)cc3)n2)ccc1NN1CCOCC1] | 2-(morpholin-4- ylamino)-5-(2-{[4- (morpholin-4- yl)phenyl]amino} pyrimidin-4- yl)benzonitrile | ¹H NMR (DMSO-d₆) δ 9.44 (s, 1H), 8.40 (d, 1H), 8.32 (d, 1H), 8.21 (dd, 1H), 8.15 (s, 1H), 7.65 (d, 2H), 7.26 (dd, 2H), 6.97 (d, 2H), 3.8-3.65 (m, 8H), 3.12-3.05 (m, 4H), 2.83 (t, 4H); LC-MS [M + H]⁺ 458.2308 |
| 286 | 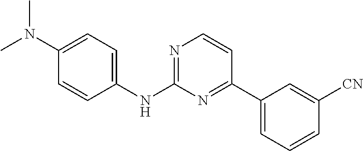 [SMILES: CN(C)c1ccc(Nc2nccc(-c3cccc(C#N)c3)n2)cc1] | 3-(2-{[4- (Dimethylamino) phenyl]amino}pyrimidin- 4-yl)benzonitrile | ¹H NMR (CD-3OD) δ 8.50 (s, 1H), 8.42 (d, 2H), 7.86-7.84 (m, 1H), 7.70-7.66 (m, 1H), 7.52-7.50 (d, 2H), 7.27 (d, 1H), 6.86-6.83 (m, 2H), 2.90 (s, 6H). LC-MS [M + H]⁺ 316.1547 |
| 287 | 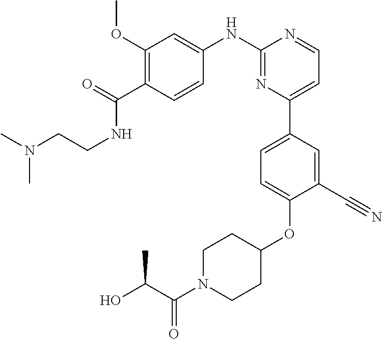 [SMILES: COc1cc(Nc2nccc(-c3ccc(OC4CCN(C(=O)[C@H](C)O)CC4)c(C#N)c3)n2)ccc1C(=O)NCCN(C)C] | 4-({4-[3-cyano-4- ({1-[(2S)-2- hydroxypropanoyl] piperidin-4- yl}oxy)phenyl] pyrimidin-2-yl} amino)-N-[2- (dimethylamino) ethyl]-2- methoxybenzamide | ¹H NMR (DMSO-d₆) δ 10.1 (s, 1H), 9.32 (br s, m1H), 8.66 (d, 1H), 8.65 (d, 1H),, 8.48 (dd, 1H), 8.41 (t, 1H), 8.00 (s, 1H), 7.88 (s, 1H), 7.60-7.57 (m, 2H), 7.37 (dd, 1H), 5.02 (br s, 2H), 4.50-4.45 (m, 1H), 4.00 (s, 3H), 3.85-3.70 (m, 2H), 3.65 (q, 2H), 3.59-3.45 (m, 2H), 3.28-3.25 (m, 2H), 2.80 (s, 6H), 2.09-1.99 (m, 2H), 1.80-1.60 (m, 2H), 1.20 (d, 3H). LC-MS [M + H]⁺ 588.2926 |
| 288 | 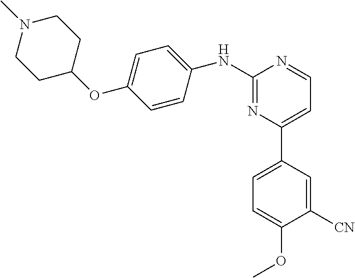 [SMILES: COc1ccc(-c2ccnc(Nc3ccc(OC4CCN(C)CC4)cc3)n2)cc1C#N] | 2-Methoxy-5-[2-({4- [(1-methylpiperidin- 4- yl)oxy]phenyl}amino) pyrimidin-4- yl]benzonitrile | ¹H NMR (CDCl₃) δ 8.43 (d, 1H), 8.29 (d, 1H), 8.26-8.23 (m, 1H), 7.53 (d, 2H), 7.20 (s, 1H), 7.07 (d, 1H), 7.02 (d, 1H), 6.96 (m, 2H), 4.30-4.29 (m, 1H), 4.00 (s, 3H), 2.76-2.88 (m, 2H), 2.38-2.27 (m, 5H), 2.05-2.00 (m, 2H), 1.91-1.83 (m, 2H). LC-MS [M + H]⁺ 416.2101 |
| 289 | 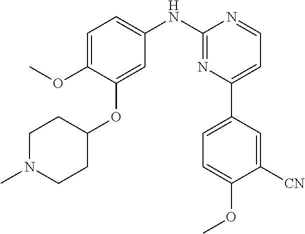 [SMILES: COc1ccc(-c2ccnc(Nc3ccc(OC)c(OC4CCN(C)CC4)c3)n2)cc1C#N] | 2-Methoxy-5-[2-({4- methoxy-3-[(1- methylpiperidin-4- yl)oxy]phenyl}amino) pyrimidin-4- yl]benzonitrile | ¹H NMR (CDCl₃) δ 8.45-8.44 (d, 1H), 8.29-8.27 (d, 1H), 8.26-8.24 (m, 1H), 7.38 (s, 1H), 7.15-7.12 (m, 2H), 7.09-7.07 (m, 2h), 6.92-6.89 (md, 2H), 4.41-4.33 (m, 1H), 4.02 (s, 3H), 3.87 (s, 3H), 3.49 (s, 3H), 2.86-2.78 (m, 2H), 2.41-2.33 (m, 2H), 2.16-2.04 (m, 2H), 2.02-1.93 (m, 2H). LC-MS [M + H]⁺ 446.3 |
| 290 | 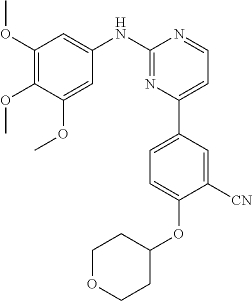 [SMILES: COc1cc(Nc2nccc(-c3ccc(OC4CCOCC4)c(C#N)c3)n2)cc(OC)c1OC] | 2-(Tetrahydro-2H- pyran-4-yloxy)-5-{2- [(3,4,5- trimethoxyphenyl) amino]pyrimidin-4- yl}benzonitrile | ¹H NMR (CDCl₃) δ 8.46 (d, 1H), 8.41 (d, 1H), 8.22-8.19 (m, 1H), 7.35 (s, 1H), 7.09-7.06 (m, 2H), 7.02 (s, 2H), 4.77-4.75 (m, 1H), 4.07-4.01 (m, 2H), 3.93 (s, 6H), 3.85 (s, 3H), 3.70-3.64 (m, 2H), 2.14-2.07 (m, 2H), 1.96-1.91 (m, 2H). LC-MS [M + H]⁺ 463.1978 |
| 291 | 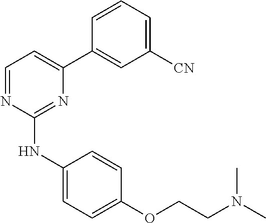 [SMILES: CN(C)CCOc1ccc(Nc2nccc(-c3cccc(C#N)c3)n2)cc1] | 3-[2-({4-[2- (Dimethylamino) ethoxy]phenyl}amino) pyrimidin-4- yl]benzonitrile | ¹H NMR (CDCl₃) δ 8.49 (d, 1H), 8.36-8.35 (m, 1H), 8.28-8.25 (m, 1H), 7.78-7.77 (m, 1H), 7.76-7.60 (m, 1H), 7.55-7.52 (m, 2H), 7.11- 7.09 (m, 2H), 6.97-6.95 (m, 2H), 4.10-4.07 (m, 2H), 2.77-2.71 (m, 2H), 2.36 (s, 6H). LC-MS [M + H]⁺ 360.1835 |
| 292 | 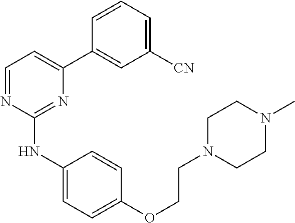 [SMILES: CN1CCN(CCOc2ccc(Nc3nccc(-c4cccc(C#N)c4)n3)cc2)CC1] | 3-[2-({4-[2-(4- Methylpiperazin-1- yl)ethoxy]phenyl} amino)pyrimidin-4- yl]benzonitrile | ¹H NMR (CD-3OD) δ 8.51-8.49 (m, 1H), 8.46-8.41 (m, 2H), 7.85-7.84 (m, 1H), 7.71-7.67 (m, 1H), 7.61- 7.58 (m, 2H), 7.30 (d, 1H), 6.95- 6.93 (m, 2H), 4.15-4.13 (m, 2H), 2.85-2.82 (m, 2H), 2.79-2.46 (m, 8H), 2.30 (s, 3H). LC-MS [M + H]⁺ 415.2232 |
| 293 | 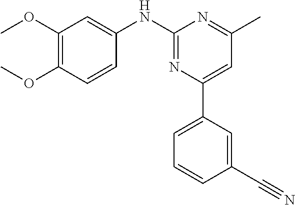 [SMILES: COc1ccc(Nc2nc(C)cc(-c3cccc(C#N)c3)n2)cc1OC] | 3-{2-[(3,4- Dimethoxyphenyl) amino]-6- methylpyrimidin-4- yl}benzonitrile | ¹H NMR (DMSO-d₆) δ 9.56 (ds, 1H), 8.60 (t, 1H), 8.51-8.44 (m, 1H), 8.04-7.98 (m, 1H), 7.80-7.20 (m, 2H), 7.44 (s, 1H), 7.19 (dd, 1H), 6.91 (d, 1H), 3.81 (s, 3H), 3.73 (s, 3H), 2.44 (s, 3H). LC-MS [M + H]⁺ 347.1529. |
| 294 | 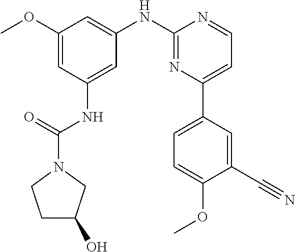 [SMILES: COc1cc(NC(=O)N2CC[C@H](O)C2)cc(Nc2nccc(-c3ccc(OC)c(C#N)c3)n2)c1] | (3S)-N-(3-{[4-(3- Cyano-4- methoxyphenyl) pyrimidin-2-yl] amino}- 5-methoxyphenyl)-3- hydroxypyrrolidine- 1-carboxamide | ¹H NMR (DMSO-d₆) δ 9.59 (s, 1H), 8.64-8.56 (m, 2H), 8.54 (d, 1H), 8.07 (s, 1H), 7.75 (s, 1H), 7.46 (d, 1H), 7.40 (d, 1H), 7.06 (t, 1H), 6.77 (t, 1H), 4.34-4.26 (m, 1H), 4.01 (s, 3H), 3.73 (s, 3H), 3.50- 3.42 (m, 3H), 3.31 (d, 1H), 2.0-1.86 (m, 1H), 1.86-1.75 (m, 1H); LC- MS [M + H]⁺ 461.1945. |
| 295 | 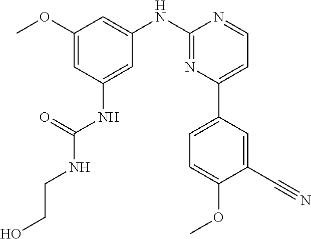 [SMILES: COc1cc(NC(=O)NCCO)cc(Nc2nccc(-c3ccc(OC)c(C#N)c3)n2)c1] | 1-(3-{[4-(3-Cyano-4- methoxyphenyl) pyrimidin-2-yl]amino}- 5-methoxyphenyl)-3- (2-hydroxyethyl)urea | ¹H NMR (DMSO-d₆) δ 9.61 (s, 1H), 8.64-8.50 (m, 4H), 7.47 (d, 1H), 7.45-7.36 (m, 2H), 7.11 (s, 1H), 6.79 (s, 1H), 6.23-6.15 (m, 1H), 4.75 (t, 1H), 4.01 (s, 3H), 3.73 (s, 3H), 3.48-3.40 (m, 2H), 3.21-3.14 (m, 2H). LC-MS [M + H]⁺ 435.1782. |
| 296 | 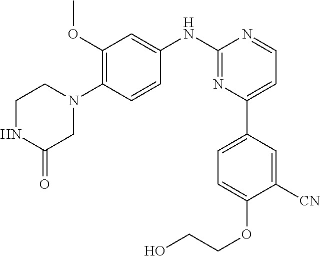 [SMILES: COc1cc(Nc2nccc(-c3ccc(OCCO)c(C#N)c3)n2)ccc1N1CCNC(=O)C1] | 2-(2- Hydroxyethoxy)-5- (2-{[3-methoxy-4-(3- oxopiperazin-1- yl)phenyl]amino} pyrimidin-4- yl)benzonitrile | ¹H NMR (CD-3OD) δ 8.41-8.38 (m, 2H), 8.31-8.28 (m, 1H), 7.67 (d, 1H), 7.24 (d, 1H), 7.17 (d, 1H), 6.92 (d, 1H), 4.28-4.25 (m, 2H), 3.98-3.96 (m, 2H), 3.94 (s, 3H), 3.69 (s, 2H), 3.45-3.42 (m, 2H), 3.28-3.26 (m, 2H). LC-MS [M + H]⁺ 461.1941 |
| 297 | 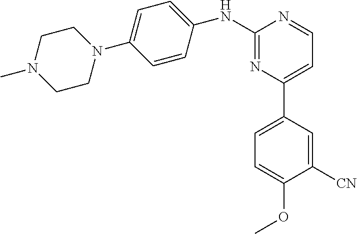 [SMILES: COc1ccc(-c2ccnc(Nc3ccc(N4CCN(C)CC4)cc3)n2)cc1C#N] | 2-Methoxy-5-(2-{[4- (4-methylpiperazin- 1- yl)phenyl]amino} pyrimidin-4- yl)benzonitrile | ¹H NMR (CDCl₃—) δ 8.43 (d, 1H), 8.30 (d, 1H), 8.27-8.24 (m, 1H), 7.53-7.51 (m, 2H), 7.07 (d, 2H), 7.06-6.96 (m, 3H), 4.01 (s, 3H), 3.21-3.19 (m, 4H), 2.61-2.59 (m, 4H), 2.37 (s, 3H). LC-MS [M + H]⁺ 401.2088 |
| 298 | 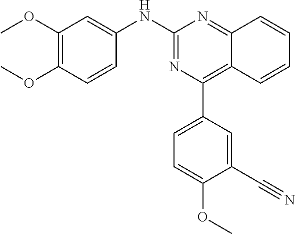 [SMILES: COc1ccc(-c2nc(Nc3ccc(OC)c(OC)c3)nc3ccccc23)cc1C#N] | 5-{2-[(3,4- Dimethoxyphenyl) amino]quinazolin- 4-yl}-2- methoxybenzonitrile | LC-MS [M + H]⁺ 413.1584. |
| 299 | 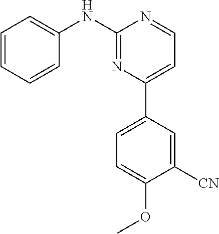 [SMILES: COc1ccc(-c2ccnc(Nc3ccccc3)n2)cc1C#N] | 2-Methoxy-5-[2- (phenylamino) pyrimidin- 4-yl]benzonitrile | ¹H NMR (CDCl₃) δ 8.47 (d, 1H), 8.31-8.27 (m, 2H), 7.68-7.66 (m, 2H), 7.40-7.36 (m, 2H), 7.10-7.06 (m, 3H), 4.02 (s, 3H). LC-MS' [M + H]⁺ 303.1243 |
| 300 | 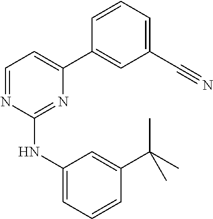 [SMILES: CC(C)(C)c1cccc(Nc2nccc(-c3cccc(C#N)c3)n2)c1] | 3-{2-[3-tert- Butylphenyl)amino] pyrimidin-4- yl}benzonitrile | ¹H NMR (CDCl₃) δ 8.53 (d, 1H), 8.44-8.33 (m, 1H), 8.31-8.28 (m, 1H), 7.80-7.76 (m, 2H), 7.63-7.59 (m, 1H), 7.45-7.43 (m, 1H), 7.38 (s, 1H), 7.34-7.30 (m, 1H), 7.26-7.12 (m, 2H), 1.38 (s, 9H). LC-MS [M + H]⁺ 329.1763 |
| 301 | 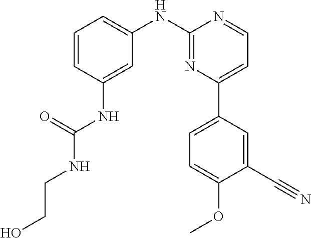 [SMILES: COc1ccc(-c2ccnc(Nc3cccc(NC(=O)NCCO)c3)n2)cc1C#N] | 1-(3-{[4-(3-Cyano-4- methoxyphenyl) pyrimidin-2- yl]amino}phenyl)-3- (2-hydroxyethyl)urea | ¹H NMR (DMSO-d₆) δ 9.63 (s, 1H), 8.64-8.58 (m, 2H), 8.56-8.50 (m, 2H), 8.05 (s, 1H), 7.47 (d, 1H), 7.41 (d, 1H), 7.24 (d, 1H), 7.13 (t, 1H), 6.98 (d, 1H), 6.20 (br s, 1H), 4.01 (s, 3H), 3.46 (t, 2H), 3.24-3.15 (m, 2H). LC-MS [M + H]⁺ 405.1663. |
| 302 | 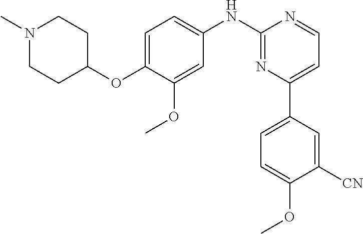 [SMILES: COc1ccc(-c2ccnc(Nc3ccc(OC4CCN(C)CC4)c(OC)c3)n2)cc1C#N] | 2-Methoxy-5-[2-({3- methoxy-4-[(1- methylpiperidin-4- yl)oxy]phenyl}amino) pyrimidin-4- yl]benzonitrile | ¹H NMR (CDCl₃) δ 8.47-8.46 (d, 1H), 8.39-8.38 (d, 1H), 8.25-8.22 (m, 1H), 7.59 (s, 1H), 7.13 (s, 1H), 7.09-7.07 (m, 2H), 7.00-6.92 (m, 2H), 4.41-4.33 (m, 1H), 4.02 (s, 3H), 3.94 (s, 3H), 3.49 (s, 3H), 3.22-3.03 (m, 1H), 2.66-2.53 (m, 3H), 2.33-2.18 (m, 2H), 2.09-2.01 (m, 2H). LC-MS [M + H]⁺ 446.3 |
| 303 | 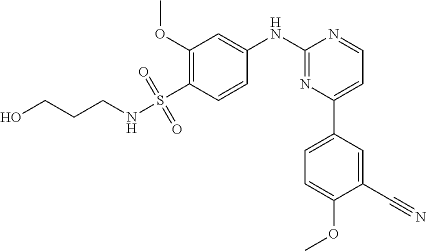 [SMILES: COc1ccc(-c2ccnc(Nc3ccc(S(=O)(=O)NCCCO)c(OC)c3)n2)cc1C#N] | 4-{[4-(3-Cyano-4- methoxyphenyl) pyrimidin-2-yl]amino}- N-(3- hydroxypropyl)-2- methoxybenzene- sulfonamide | LC-MS [M + H]⁺ 470.1474 |
| 304 | 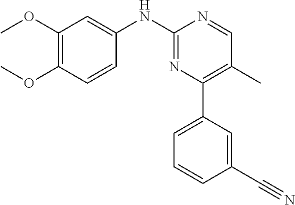 [SMILES: COc1ccc(Nc2ncc(C)c(-c3cccc(C#N)c3)n2)cc1OC] | 3-{2-[(3,4- Dimethoxyphenyl) amino]-5- methylpyrimidin-4- yl}benzonitrile | ¹H NMR (DMSO-d₆) δ 9.46 (s, 1H), 8.43 (s, 1H), 8.18 (s, 1H), 8.05 (d, 1H), 7.98 (d, 1H), 7.74 (t, 1H), 7.67 (br s, 1H), 3.73 (s, 3H), 3.70 (s, 3H), 2.23 (s, 3H). LC-MS [M + H]⁺ 347.1532. |
| 305 | 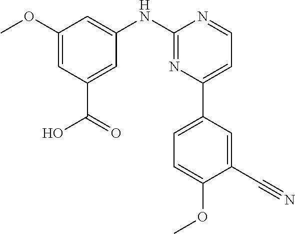 [SMILES: COc1cc(Nc2nccc(-c3ccc(OC)c(C#N)c3)n2)cc(C(=O)O)c1] | 3-{[4-(3-Cyano-4- methoxyphenyl) pyrimidin-2-yl]amino}- 5-methylbenzoic acid | ¹H NMR (DMSO-d₆) δ 9.57 (s, 1H), 8.60-8.50 (m, 3H), 7.77 (s, 1H), 7.56 (t, 1H), 7.47-7.40 (m, 2H), 7.07 (dd, 1H), 4.02 (s, 3H), 3.77 (s, 3H). LC-MS [M + H]⁺ 377.1245. |
| 306 | 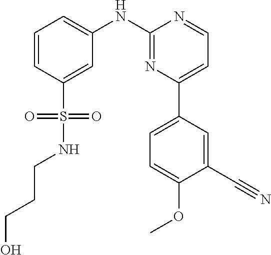 [SMILES: COc1ccc(-c2ccnc(Nc3cccc(S(=O)(=O)NCCCO)c3)n2)cc1C#N] | 3-{[4-(3-Cyano-4- methoxyphenyl) pyrimidin-2-yl]amino}- N-(3- hydroxypropyl) benzenesulfonamide | ¹H NMR (DMSO-d₆) δ 10.10 (s, 1 H), 8.52-8.68 (m, 4 H), 7.82- 7.93 (m, 1 H), 7.45-7.63 (m, 3 H), 7.31-7.45 (m, 2 H), 4.42 (t, 1 H), 4.02 (s, 3 H), 3.27-3.45 (m, 2 H), 2.74-2.93 (m, 2 H), 1.46-1.63 (m, 2 H). |
| 307 | 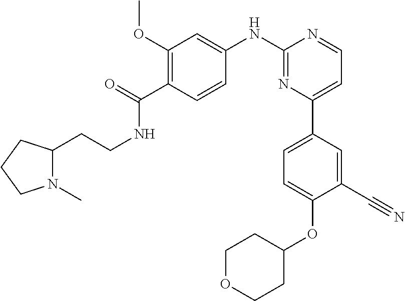 [SMILES: COc1cc(Nc2nccc(-c3ccc(OC4CCOCC4)c(C#N)c3)n2)ccc1C(=O)NCCC1CCCN1C] | 4-({4-[3-cyano-4- (tetrahydro-2H- pyran-4- yloxy)phenyl] pyrimidin-2-yl} amino)-2- methoxy-N-[2-(1- methylpyrrolidin-2- yl)ethyl]benzamide | ¹H NMR (DMSO-d₆) δ 10.1 (s, 1H), 9.40 (br s, 1H), 8.63 (d, 1H), 8.61 (d, 1H), 8.46 (dd, 1H), 8.25 (t, 1H), 7.96 (s, 1H), 7.81 (d, 1H), 7.58- 7.55 (m, 2H), 7.36 (dd, 1H), 4.99- 4.93 (m, 1H), 3.99 (s, 3H), 3.90- 3.85 (m, 2H), 3.59-3.53 (m, 3H), 3.38 (q, 2H), 3.38-3.18 (m, 1H), 3.10-3.03 (m, 1H), 2.84 (d, 3H), 2.39-2.31 (m, 1H), 2.20-2.13 (m, 1H), 2.081.97 (m, 4H), 1.93-1.86 (m, 1H), 1.76-1.63 (m, 3H). LC- MS [M + H]⁺ 557.2854 |
| 308 | 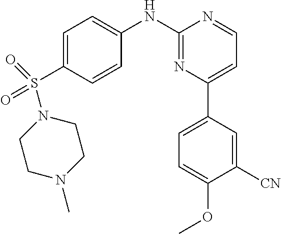 [SMILES: COc1ccc(-c2ccnc(Nc3ccc(S(=O)(=O)N4CCN(C)CC4)cc3)n2)cc1C#N] | 2-Methoxy-5-[2-({4- [(4-methylpiperazin- 1- yl)sulfonyl]phenyl} amino)pyrimidin-4- yl]benzonitrile | ¹H NMR (DMSO-d₆) δ 8.66 (d, 1H), 8.59-8.53 (m, 2H), 8.15-8.12 (m, 2H), 7.75 (d, 2H), 7.64 (d, 1H), 7.46 (d, 1H), 4.20-3.40 (m, 8H), 4.03 (s, 3H), 2.79 (s, 3H). LC-MS [M + H]⁺ 465.1710 |
| 309 | 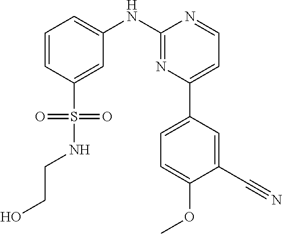 [SMILES: COc1ccc(-c2ccnc(Nc3cccc(S(=O)(=O)NCCO)c3)n2)cc1C#N] | 3-{[4-(3-Cyano-4- methoxyphenyl) pyrimidin-2-yl]amino}- N-(2- hydroxyethyl) benzenesulfonamide | ¹H NMR (DMSO-d₆) δ 10.10 (s, 1 H), 8.56-8.66 (m, 3 H), 7.85- 7.96 (m, 1 H), 7.50-7.60 (m, 3 H), 7.36-7.44 (m, 2 H), 4.69 (t, 1 H), 4.02 (s, 3 H), 3.38 (q, 1 H), 2.84 (q, 2 H) |
| 310 | 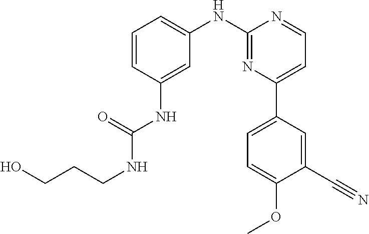 [SMILES: COc1ccc(-c2ccnc(Nc3cccc(NC(=O)NCCCO)c3)n2)cc1C#N] | 1-(3-{[4-(3-Cyano-4- methoxyphenyl) pyrimidin-2- yl]amino}phenyl)-3- (3- hydroxypropyl)urea | ¹H NMR (DMSO-d₆) δ 9.63 (s, 1H), 8.65-8.57 (m, 2H), 8.54 (d, 1H), 8.55 (s, 1H), 8.03 (s, 1H), 7.47 (d, 1H), 7.41 (d, 1H), 7.24 (d, 1H), 7.13 (t, 1H), 6.99 (d, 1H), 6.12 (br s, 1H), 4.01 (s, 3H), 3.46 (t, 2H), 3.22-3.12 (m, 2H), 1.59 (quint., 2H). LC-MS [M + H]⁺ 419.1829. |
| 311 | 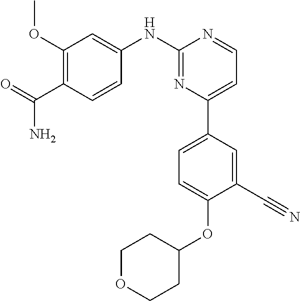 [SMILES: COc1cc(Nc2nccc(-c3ccc(OC4CCOCC4)c(C#N)c3)n2)ccc1C(N)=O] | 4-({4-[3-cyano-4- (tetrahydro-2H- pyran-4- yloxy)phenyl] pyrimidin-2-yl} amino)-2- methoxybenzamide | ¹H NMR (DMSO-d₆) δ 10.1 (s, 1H), 8.63 (d, 1H), 8.60 (d, 1H), 8.48 (dd, 1H), 7.92 (d, 1H), 7.85 (d, 1H), 7.58-7.56 (m, 3H), 7.40-7.36 (m, 2H), 4.99-4.93 (m, 1H), 3.97 (s, 3H), 3.90-3.85 (m, 2H), 3.59-3.53 (m, 2H), 2.09-2.01 (m, 2H), 1.80- 1.60 (m, 2H), 1.74-1.65 (m, 2H). LC-MS [M + Na]⁺ 468.1629 |
| 312 | 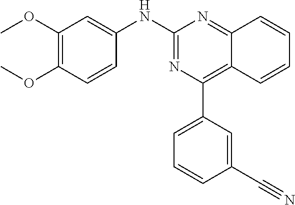 [SMILES: COc1ccc(Nc2nc(-c3cccc(C#N)c3)c3ccccc3n2)cc1OC] | 3-{2-[(3,4- Dimethoxyphenyl) amino]quinazolin-4- yl}benzonitrile | ¹H NMR (DMSO-d₆) δ 9.85 (s, 1H), 8.28-8.24 (m, 1H), 8.14-8.08 (m, 2H), 7.90-7.80 (m, 3H), 7.79-7.70 (m, 2H), 7.48-7.38 (m, 1H), 7.38- 7.30 (m, 1H), 6.93 (d, 1H), 3.80 (s, 3H), 3.74 (s, 3H); LC-MS [M + H]⁺ 383.1501. |
| 313 | 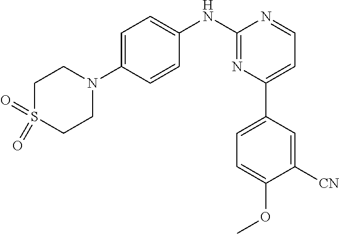 [SMILES: COc1ccc(-c2ccnc(Nc3ccc(N4CCS(=O)(=O)CC4)cc3)n2)cc1C#N] | 5-(2-{[4-(1,1- Dioxidothiomorpholin- 4- yl)phenyl]amino} pyrimidin-4-yl)-2- methoxybenzonitrile] | ¹H NMR (CDCl₃) δ 8.45 (d, 1H), 8.32 (d, 1H), 8.25-8.22 (m, 1H), 7.59-7.56 (m, 2H), 7.18 (s, 1H), 7.09-7.05 (m, 2H), 6.99-6.96 (m, 2H), 4.01 (s, 3H), 3.82-3.79 (m, 4H), 3.17-3.14 (m, 4H). LC-MS [M + H]⁺ 436.2 |
| 314 | 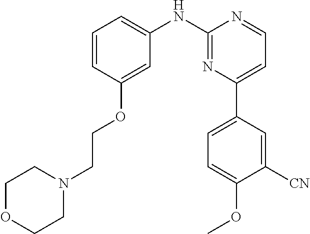 [SMILES: COc1ccc(-c2ccnc(Nc3cccc(OCCN4CCOCC4)c3)n2)cc1C#N] | 2-Methoxy-5-[2-({3- [2-(morpholin-4- yl)ethoxy]phenyl} amino)pyrimidin-4- yl]benzonitrile | ¹H NMR (CD-3OD) δ 8.46-8.43 (m, 2H), 8.41-8.38 (m, 1H), 7.65-7.63 (m, 1H), 7.31-7.26 (m, 2H), 7.22- 7.14 (m, 2H), 6.62-6.59 (m, 1H), 4.20-4.17 (m, 2H), 4.03 (s, 3H), 3.72-3.70 (m, 4H), 2.87-2.84 (m, 2H), 2.63-2.61 (m, 4H). LC-MS [M + H]⁺ 432.2030 |
| 315 | 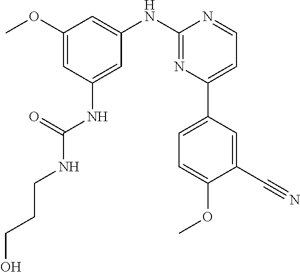 [SMILES: COc1cc(NC(=O)NCCCO)cc(Nc2nccc(-c3ccc(OC)c(C#N)c3)n2)c1] | 1-(3-{[4-(3-Cyano-4- methoxyphenyl) pyrimidin-2-yl] amino}- 5-methoxyphenyl)-3- (3- hydroxypropyl)urea | ¹H NMR (DMSO-d₆) δ 9.61 (s, 1H), 8.62-8.55 (m, 2H), 8.54 (d, 1H), 8.44 (s, 1H), 7.47 (d, 1H), 7.45- 7.38 (m, 2H), 7.11 (t, 2H), 6.80 (s, 1H), 6.12 (t, 1H), 4.50 (t, 1H), 4.01 (s, 3H), 3.73 (s, 3H), 3.46 (q, 2H), 3.16 (q, 2H), 1.59 (quint., 2H), LC-MS [M + H]⁺ 449.1950. |
| 316 | 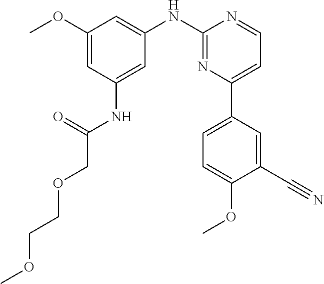 [SMILES: COCCOCC(=O)Nc1cc(Nc2nccc(-c3ccc(OC)c(C#N)c3)n2)cc(OC)c1] | N-(3-{[4-(3-Cyano- 4- methoxyphenyl) pyrimidin-2-yl] amino}- 5-methoxyphenyl)-2- (2- methoxyethoxy) acetamide | ¹H NMR (DMSO-d₆) δ 9.73 (s, 1H), 9.56 (s, 1H), 8.62 (d, 1H), 8.58- 8.52 (m, 2H), 7.88 (s, 1H), 7.49 (d, 1H), 7.41 (d, 1H), 7.20 (s, 1H), 6.89 (s, 1H), 4.10 (s, 2H), 4.02 (s, 3H), 3.76 (s, 3H), 3.74-3.65 (m, 2H), 3.56-3.50 (m, 2H), 3.31 (s, 3H); LC-MS [M + H]⁺ 464.1935. |
| 317 | 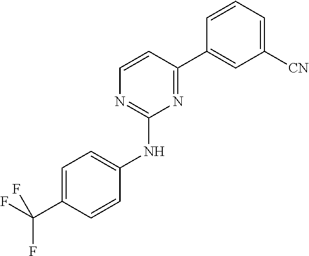 [SMILES: N#Cc1cccc(-c2ccnc(Nc3ccc(C(F)(F)F)cc3)n2)c1] | 3-(2-{[4- (Trifluoromethyl) phenyl]amino} pyrimidin- 4-yl)benzonitrile | ¹H NMR (CD-3OD) δ 8.59 (d, 1H), 8.54 (s, 1H), 8.46 (d, 1H), 7.97 (d, 2H), 7.88 (d, 1H), 7.74-7.71 (m, 1H), 7.65-7.56 (m, 3H), 7.43 (d, 1H). LC-MS [M + H]⁺ 341.1022 |
| 318 | 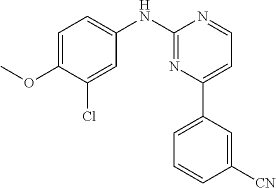 [SMILES: COc1ccc(Nc2nccc(-c3cccc(C#N)c3)n2)cc1Cl] | 3-{2-[(3-Chloro-4- methoxyphenyl) amino]pyrimidin-4- yl}benzonitrile | ¹H NMR (CDCl₃) δ 8.52 (d, 1H), 8.36-8.35 (m, 1H), 8.29-8.26 (m, 1H), 7.81 (d, 1H), 7.79-7.77 (m, 1H), 7.76-7.60 (m, 1H), 7.46-7.43 (m, 1H), 7.21 (s, 1H), 7.14 (d, 1H), 6.95 (d, 1H), 3.92 (s, 3H). LC-MS [M + H]⁺ 337.0857 |
| 319 | 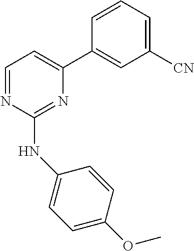 [SMILES: COc1ccc(Nc2nccc(-c3cccc(C#N)c3)n2)cc1] | 3-{2-[(4- Methoxyphenyl) amino]pyrimidin-4- yl}benzonitrile | ¹H NMR (CDCl₃) δ 8.46 (d, 1H), 8.36 (s, 1H), 8.29 (d, 1H), 7.79 (d, 1H), 7.66-7.62 (m, 1H), 7.56 (d, 2H), 7.12 (d, 1H), 6.95 (d, 2H), 3.84 (s, 3H). LC-MS [M + H]⁺ 303.1244 |
| 320 | 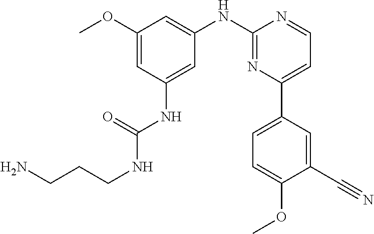 [SMILES: COc1cc(NC(=O)NCCCN)cc(Nc2nccc(-c3ccc(OC)c(C#N)c3)n2)c1] | 1-(3-Aminopropyl)- 3-(3-{[4-(3-cyano-4- methoxyphenyl) pyrimidin-2-yl] amino}-5- methoxyphenyl)urea | ¹H NMR (DMSO-d₆) δ 9.63 (s, 1H), 8.63-8.52 (m, 4H), 7.70 (s, 3H), 7.48 (s, 1H), 7.44-7.36 (m, 2H), 7.13 (t, 1H), 6.83 (t, 1H), 6.33 (t, 1H), 4.02 (s, 3H), 3.73 (s, 3H), 3.23-3.15 (m, 2H), 2.89-2.76 (m, 2H), 1.78-1.66 (m, 2H). LC-MS [M + H]⁺ 448.2085. |
| 321 | 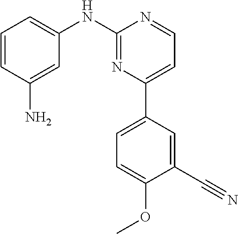 [SMILES: COc1ccc(-c2ccnc(Nc3cccc(N)c3)n2)cc1C#N] | 5-{2-[(3- Aminophenyl)amino] pyrimidin-4-yl}-2- methoxybenzonitrile | ¹H NMR (DMSO-d₆) δ 9.98 (s, 1H), 8.60 (d, 1H), 8.57 (d, 1H), 8.53 (dd, 1H), 7.89 (s, 1H), 7.65 (d, 1H), 7.54 (d, 1H), 7.44 (d, 1H), 7.36 (t, 1H), 6.84 (d, 1H), 4.02 (s, 3H). LC-MS [M + H]⁺ 318.1338. |
| 322 | 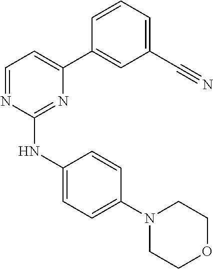 [SMILES: N#Cc1cccc(-c2ccnc(Nc3ccc(N4CCOCC4)cc3)n2)c1] | 3-(2-{[4-(Morpholin- 4- yl)phenyl]amino} pyrimidin-4- yl)benzonitrile | ¹H NMR (CDCl₃) δ 8.49 (d, 1H), 8.37 (s, 1H), 8.26 (d, 1H), 7.77 (d, 1H), 7.63-7.54 (m, 3H), 7.18 (s, 1H), 7.09 (d, 1H), 6.96 (d, 2H), 3.90-3.87 (m, 4H), 3.16-3.14 (m, 4H). LC-MS [M + H]⁺ 358.1640 |
| 323 | 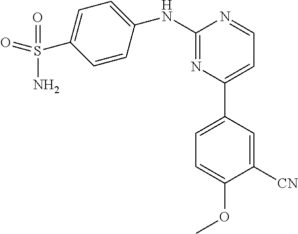 [SMILES: COc1ccc(-c2ccnc(Nc3ccc(S(N)(=O)=O)cc3)n2)cc1C#N] | 4-{[4-(3-Cyano-4- methoxyphenyl) pyrimidin-2- yl]amino} benzenesulfonamide | ¹H NMR (DMSO-d₆) δ 10.13 (s, 1H), 8.63 (d, 1H), 8.57 (s, 1H), 8.57-8.51 (m, 1H), 7.97 (d, 2H), 7.77 (d, 2H), 7.58 (d, 1H), 7.46 (d, 1H), 7.20 (s, 2H), 4.02 (s, 3H). LC-MS [M + H]⁺ 382.0946 |
| 324 | AND Enantiomer 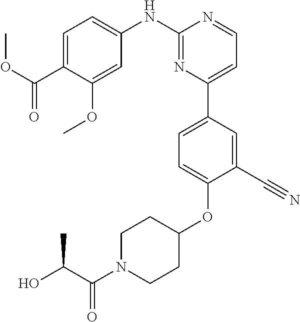 [SMILES: COC(=O)c1ccc(Nc2nccc(-c3ccc(OC4CCN(C(=O)[C@H](C)O)CC4)c(C#N)c3)n2)cc1OC] | methyl 4-({4-[3- cyano-4-({1-[(2S)-2- hydroxypropanoyl] piperidin-4- yl}oxy)phenyl] pyrimidin-2-yl} amino)-2- methoxybenzoate | ¹H NMR (DMSO-d₆) δ 10.1 (s, 1H), 8.64 (d, 1H), 8.60 (d, 1H), 8.49 (dd, 1H), 7.90 (s, 1H), 7.71 (d, 1H), 7.60-7.57 (m, 2H), 7.39 (d, 1H), 5.05-4.96 (m, 2H), 4.49-4.44 (m, 1H), 3.87 (s, 3H), 3.86-3.65 (m, 2H), 3.75 (s, 3H), 3.59-3.53 (m, 2H), 2.09-1.95 (m, 2H), 1.80-1.60 (m, 2H). LC-MS [M + H]⁺ 532.2203 |
| 325 | 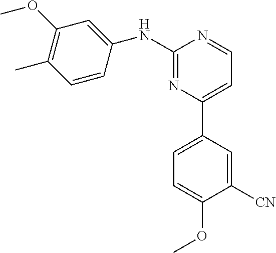 [SMILES: COc1cc(Nc2nccc(-c3ccc(OC)c(C#N)c3)n2)ccc1C] | 2-Methoxy-5-{2-[(3- methoxy-4- methylphenyl)amino] pyrimidin-4- yl}benzonitrile | ¹H NMR (CDCl₃) δ 8.46 (d, 1H), 8.38 (d, 1H), 8.28-8.25 (m, 1H), 7.50 (d, 1H), 7.15 (s, 1H), 7.11- 7.05 (m, 3H), 6.97-6.95 (m, 1H), 4.02 (s, 3H), 3.91 (s, 3H), 2.21 (s, 3H). LC-MS [M + H]⁺ 347.1504 |
| 326 | 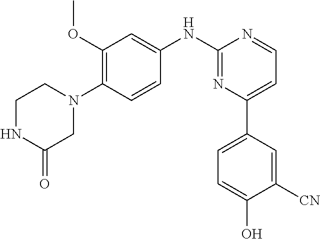 [SMILES: COc1cc(Nc2nccc(-c3ccc(O)c(C#N)c3)n2)ccc1N1CCNC(=O)C1] | 2-Hydroxy-5-(2-{[3- methoxy-4-(3- oxopiperazin-1- yl)phenyl]amino} pyrimidin-4- yl)benzonitrile | ¹H NMR (CDCl₃) δ 8.39 (d, 1H), 8.28 (d, 1H), 8.12-8.09 (m, 1H), 7.63 (d, 1H), 7.10-7.06 (m, 2H), 6.99 (d, 1H), 6.92 (d, 1H), 3.97 (s, 3H), 3.77 (s, 2H), 3.49-3.42 (m, 2H), 3.33-3.31 (m, 2H). LC-MS [M + H]⁺ 417.1663 |
| 327 | 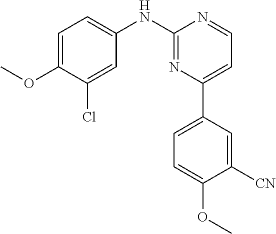 [SMILES: COc1ccc(Nc2nccc(-c3ccc(OC)c(C#N)c3)n2)cc1Cl] | 5-{2-[(3-Chloro-4- methoxyphenyl) amino] pyrimidin-4-yl}-2- methoxybenzonitrile | ¹H NMR (CDCl₃) δ 8.46 (d, 1H), 8.29-8.26 (m, 2H), 7.83 (d, 1H), 7.44-7.41 (m, 1H), 7.17 (s, 1H), 7.10-7.06 (m, 2H), 6.94 (d, 1H), 4.02 (s, 3H), 3.91 (s, 3H). LC-MS [M + H]⁺ 367.0965 |
| 328 | 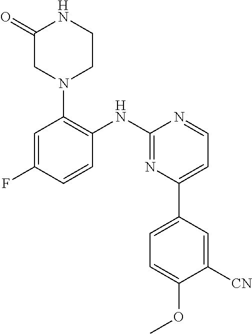 [SMILES: COc1ccc(-c2ccnc(Nc3ccc(F)cc3N3CCNC(=O)C3)n2)cc1C#N] | 5-(2-{[4-Fluoro-2-(3- oxopiperazin-1- yl)phenyl]amino} pyrimidin-4-yl)-2- methoxybenzonitrile | ¹H NMR (CDCl₃) δ 8.82-8.33 (m, 1H), 8.65 (d, 1H), 8.21 (d, 1H), 8.17-8.14 (m, 1H), 7.50-7.11 (m, 1H), 7.33 (d, 1H), 7.02 (d, 1H), 6.74 (d, 1H), 4.37-4.34 (m, 1H), 4.28 (s, 1H), 4.03 (s, 2H), 4.02 (s, 3H), 3.98-3.88 (m, 1H), 3.79-3.75 (m, 1H). LC-MS [M + H]⁺ 419.1438 |
| 329 | 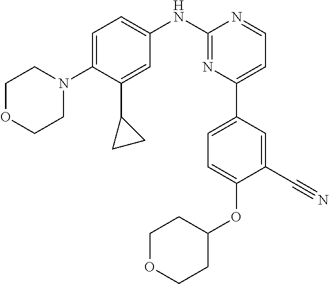 [SMILES: N#Cc1cc(-c2ccnc(Nc3ccc(N4CCOCC4)c(C4CC4)c3)n2)ccc1OC1CCOCC1] | 5-(2-{[3- cyclopropyl-4- (morpholin-4- yl)phenyl]amino} pyrimidin-4-yl)-2- (tetrahydro-2H- pyran-4- yloxy)benzonitrile | ¹H NMR (DMSO-d₆) δ 9.46 (s, 1H), 8.51 (d, 1H), 8.49 (s, 1H), 8.41 (dd, 1H), 7.53 (d, 1H), 7.48 (dd, 1H), 7.41 (d, 1H), 7.29 (s, 1H), 6.99 (d, 1H), 4.95 (hep, 1H), 3.91- 3.84 (m, 2H), 3.76 (t, 4H), 3.6-3.52 (m, 2H), 2.9 (t, 4H), 2.37-2.29 (m, 1H), 2.09-2.0 (m, 2H), 1.75-1.64 (m, 2H), 1.04-0.99 (m, 2H), 0.7- 0.64 (m, 2H); LC-MS [M + H]⁺ 498.2368 |
| 330 | 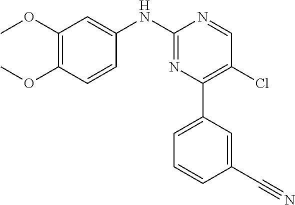 [SMILES: COc1ccc(Nc2ncc(Cl)c(-c3cccc(C#N)c3)n2)cc1OC] | 3-{5-Chloro-2-[(3,4- dimethoxyphenyl) amino]pyrimidin-4- yl}benzonitrile | ¹H NMR (DMSO-d₆) δ 9.85 (s, 1H), 8.64 (s, 1H), 8.28 (s, 1H), 8.17 (d, 1H), 8.03 (dd, 1H), 7.77 (t, 1H), 7.59 (br s, 1H), 7.15 (d, 1H), 6.89 (d, 1H), 3.73 (s, 3H), 3.71 (s, 3H). LC-MS [M + H]⁺ 367.0957. |
| 331 | 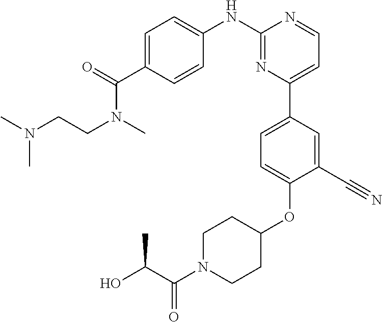 [SMILES: C[C@H](O)C(=O)N1CCC(Oc2ccc(-c3ccnc(Nc4ccc(C(=O)N(C)CCN(C)C)cc4)n3)cc2C#N)CC1] | 4-({4-[3-cyano-4- ({1-[(2S)-2- hydroxypropanoyl] piperidin-4- yl}oxy)phenyl] pyrimidin- 2-yl}amino)-N- [2- (dimethylamino) ethyl]-N- methylbenzamide | ¹H NMR (DMSO-d₆) δ 10.0 (s, 1H), 9.32 (br s, 1H), 8.61 (d, 1H), 8.57 (d, 1H), 8.48 (dd, 1H), 7.90 (d, 2H), 7.58-7.55 (m, 2H), 7.47 (d, 2H), 5.02 (br s, 2H), 4.47 (q, 1H), 3.85-3.68 (m, 4H), 3.65 (q, 2H), 3.59-3.47 (m, 2H), 3.45-3.37 (m, 2H), 3.02 (s, 3H), 2.87 (s, 6H), 2.09-1.93 (m, 2H), 1.80-1.60 (m, 2H), 1.20 (d, 3H). LC-MS [M + H]⁺ 572.2970 |
| 332 | 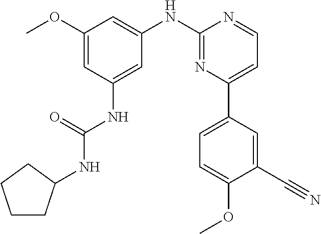 [SMILES: COc1cc(NC(=O)NC2CCCC2)cc(Nc2nccc(-c3ccc(OC)c(C#N)c3)n2)c1] | 1-(3-{[4-(3-Cyano-4- methoxyphenyl) pyrimidin-2-yl] amino}- 5-methoxyphenyl)-3- cyclopentylurea | ¹H NMR (DMSO-d₆) δ 9.62 (s, 1H), 8.65-8.55 (m, 2H), 8.54 (d, 1H), 8.25 (s, 1H), 7.48 (d, 1H), 7.42 (d, 1H), 7.35 (s, 1H), 7.12 (s, 1H), 6.81 (s, 1H), 6.14 (d, 1H), 4.01 (s, 3H), 3.97 (q, 1H), 3.73 (s, 3H), 1.90-1.80 (m, 2H), 1.70-1.58 (m, 2H), 1.58-1.47 (m, 2H), 1.42-1.30 (m, 2H). LC-MS [M + H]⁺ 459.2143 |
| 333 | 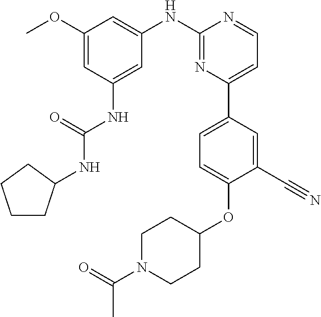 [SMILES: COc1cc(NC(=O)NC2CCCC2)cc(Nc2nccc(-c3ccc(OC4CCN(C(C)=O)CC4)c(C#N)c3)n2)c1] | 1-{3-[(4-{4-[(1- Acetylpiperidin-4- yl)oxy]-3- cyanophenyl} pyrimidin- 2-yl)amino]-5- methoxyphenyl}-3- cyclopentylurea | ¹H NMR (DMSO-d₆) δ 9.62 (s, 1H), 8.60 (d, 1H), 8.55 (d, 1H), 8.52 (d, 1H), 8.28 (s, 1H), 7.53 (d, 1H), 7.48 (d, 1H), 7.36 (s, 1H), 7.12 (s, 1H), 6.81 (s, 1H), 6.17 (d, 1H), 5.02-4.94 (m, 1H), 4.01-3.92 (m, 1H), 3.73 (s, 3H), 3.76-3.60 (m, 2H), 3.48-3.38 (m, 2H), 2.04 (s, 3H), 2.08-2.00 (m, 2H), 1.98-1.70 (m, 4H), 1.68-1.45 (m, 5H), 1.40- 1.30 (m, 2H). LC-MS [M + H]⁺ 570.2823 |
| 334 | 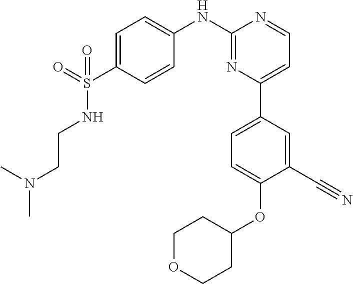 [SMILES: CN(C)CCNS(=O)(=O)c1ccc(Nc2nccc(-c3ccc(OC4CCOCC4)c(C#N)c3)n2)cc1] | 4-({4-[3-cyano-4- (tetrahydro-2H- pyran-4- yloxy)phenyl] pyrimidin-2-yl} amino)-N- [2- (dimethylamino) ethyl] benzenesulfonamide | ¹H NMR (DMSO-d₆) δ 10.2 (s, 1H), 9.35 (br s, 1H), 8.65 (d, 12H), 8.58 (d, 1H), 8.49 (dd, 1H), 8.06 (d, 2H), 7.80-7.77 (m, 3H), 7.62 (d, 1H), 7.57 (d, 1H), 4.99-4.93 (m, 1H), 3.91-3.85 (m, 2H), 3.59-3.53 (m, 2H), 3.18-3.12 (m, 2H), 3.07- 3.03 (m, 2H), 2.79 (s, 6H), 2.09- 2.03 (m, 2H), 1.74-1.65 (m, 2H). LC-MS [M + H]⁺ 523.2121 |
| 335 | 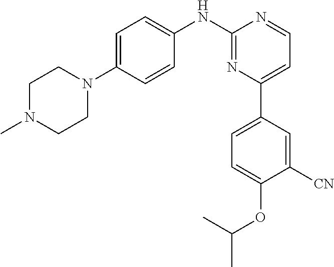 [SMILES: CC(C)Oc1ccc(-c2ccnc(Nc3ccc(N4CCN(C)CC4)cc3)n2)cc1C#N] | 5-(2-{[4-(4- Methylpiperazin-1- yl)phenyl]amino} pyrimidin-4-yl)-2- (propan-2- yloxy)benzonitrile | ¹H NMR (CDCl₃) δ 8.42 (d, 1H), 8.27 (d, 1H), 8.23-8.21 (m, 1H), 7.54-7.51 (m, 2H), 7.10 (s, 1H), 7.05 (d, 1H), 7.01-6.95 (m, 3H), 4.77-4.71 (m, 1H), 3.21-3.19 (m, 4H), 2.62-2.58 (m, 4H), 2.36 (s, 3H), 1.45 (d, 6H). LC-MS [M + H]⁺ 429.1170 |
| 336 | 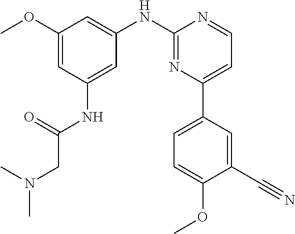 [SMILES: COc1cc(NC(=O)CN(C)C)cc(Nc2nccc(-c3ccc(OC)c(C#N)c3)n2)c1] | N-(3-{[4-(3-Cyano- 4- methoxyphenyl) pyrimidin-2-yl] amino}- 5-methoxyphenyl)- N~2~,N~2~- dimethylglycinamide | ¹H NMR (DMSO-d₆) δ 10.03 (s, 1H), 9.74 (s, 1H), 8.61 (d, 1H), 8.57-8.55 (m, 2H), 7.84 (s, 1H), 7.50 (d, 1H), 7.42 (d, 1H), 7.24 (s, 1H), 6.89 (s, 1H), 4.02 (s, 3H), 3.76 (s, 3H), 3.53 (br s, 2H), 2.55 (s, 6H); LC-MS [M + H]⁺ 433.1965. |
| 337 | 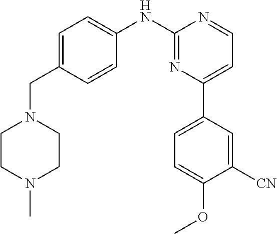 [SMILES: COc1ccc(-c2ccnc(Nc3ccc(CN4CCN(C)CC4)cc3)n2)cc1C#N] | 2-Methoxy-5-[2-({4- [(4-methylpiperazin- 1- yl)methyl]phenyl} amino)pyrimidin-4- yl]benzonitrile | ¹H NMR (CDCl₃—) δ 8.47 (d, 1H), 8.31-8.27 (m, 2H), 7.63-7.61 (m, 2H), 7.33-7.31 (m, 2H), 7.11-7.06 (m, 2H), 4.02 (s, 3H), 3.51 (s, 2H), 2.70-2.33 (m, 8H), 2.31 (s, 3H). LC-MS [M + H]⁺ 415.2245 |
| 338 | 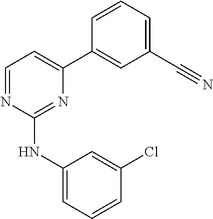 [SMILES: N#Cc1cccc(-c2ccnc(Nc3cccc(Cl)c3)n2)c1] | 3-{2-[(3- Chlorophenyl)amino] pyrimidin-4- yl}benzonitrile | ¹H NMR (CDCl₃) δ 8.56 (d, 1H), 8.38-8.37 (m, 1H), 8.31-8.28 (m, 1H), 7.94-7.93 (m, 1H), 7.80-7.77 (m, 1H), 7.66-7.62 (m, 1H), 7.45- 7.42 (m, 1H), 7.38 (s, 1H), 7.30- 7.26 (m, 1H), 7.19 (d, 1H), 7.06- 7.04 (m, 1H). LC-MS [M + H]⁺ 307.0753 |
| 339 | 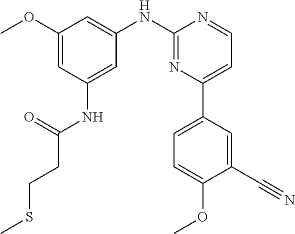 [SMILES: COc1cc(NC(=O)CCSC)cc(Nc2nccc(-c3ccc(OC)c(C#N)c3)n2)c1] | N-(3-{[4-(3-Cyano- 4- methoxyphenyl) pyrimidin-2-yl] amino}- 5-methoxyphenyl)-3- (methylsulfanyl) propanamide | LC-MS [M + H]⁺ 450.1588. |
| 340 | 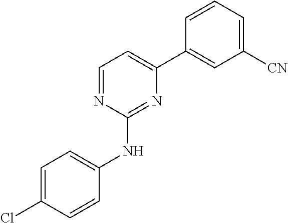 [SMILES: N#Cc1cccc(-c2ccnc(Nc3ccc(Cl)cc3)n2)c1] | 3-{2-[(4- Chlorophenyl)amino] pyrimidin-4- yl}benzonitrile | ¹H NMR (CDCl₃) δ 8.54 (d, 1H), 8.36-8.35 (m, 1H), 8.29-8.26 (m, 1H), 7.80-7.78 (m, 1H), 7.65-7.61 (m, 3H), 7.36-7.32 (m, 2H), 7.25 (s, 1H), 7.17 (d, 1H). LC-MS [M + H]⁺ 307.0752 |
| 341 | 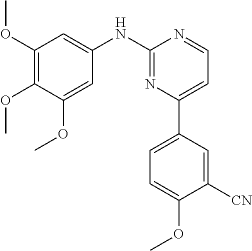 [SMILES: COc1ccc(-c2ccnc(Nc3cc(OC)c(OC)c(OC)c3)n2)cc1C#N] | 2-methoxy-5-{2- [(3,4,5- trimethoxyphenyl) amino]pyrimidin-4- yl}benzonitrile | ¹H NMR (CDCl₃) δ 8.47 (d, 1H), 8.39 (d, 1H), 8.26-8.23 (m, 1H), 7.18 (s, 1H), 7.09-7.06 (m, 2H), 7.02 (s, 2H), 4.02 (s, 3H), 3.93 (s, 6H), 3.85 (s, 3H). LC-MS [M + H]⁺ 393.1587 |
| 342 | AND Enantiomer 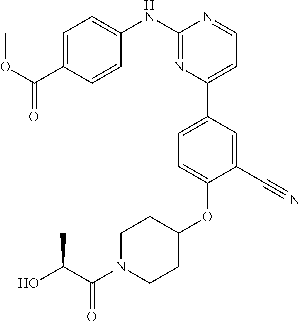 [SMILES: COC(=O)c1ccc(Nc2nccc(-c3ccc(OC4CCN(C(=O)[C@H](C)O)CC4)c(C#N)c3)n2)cc1] | methyl 4-({4-[3- cyano-4-({1-[(2S)-2- hydroxypropanoyl] piperidin-4- yl}oxy)phenyl] pyrimidin-2- yl}amino)benzoate | ¹H NMR (DMSO-d₆) δ 10.2 (s, 1H), 8.65 (dd, 1H), 8.59 (d, 1H), 8.51 (d, 1H), 7.9-7.94 (m, 4H), 7.61- 7.58 (d, 2H), 5.01 (br s, 1H), 4.48- 4.45 (m, 1H), 3.83 (s, 3H), 3.83- 3.70 (m, 2H), 3.60-3.52 (m, 2H), 2.09-1.95 (m, 2H), 1.82-1.60 (m, 2H), 1.20 (d, 3H). LC-MS [M + H]⁺ 502.2081 |
| 343 | 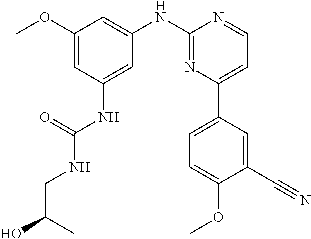 [SMILES: COc1cc(NC(=O)NC[C@@H](C)O)cc(Nc2nccc(-c3ccc(OC)c(C#N)c3)n2)c1] | 1-(3-{[4-(3-Cyano-4- methoxyphenyl) pyrimidin-2-yl]amino}- 5-methoxyphenyl)-3- [(2R)-2- hydroxypropyl]urea | ¹H NMR (DMSO-d₆) δ 9.61 (s, 1H), 8.62-8.50 (m, 4H), 7.47 (d, 1H), 7.45-7.36 (m, 2H), 7.11 (t, 1H), 6.80 (s, 1H), 6.18 (t, 1H), 4.77 (d, 1H) 4.02 (s, 3H), 3.73 (s, 3H), 3.70-3.64 (m, 1H), 3.20-3.11 (m, 1H), 3.00-2.90 (m, 1H), 1.06 (d, 1H). LC-MS [M + H]⁺ 449.1936. |
| 344 | 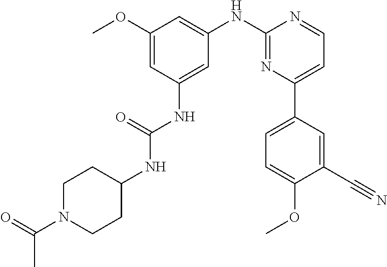 [SMILES: COc1cc(NC(=O)NC2CCN(C(C)=O)CC2)cc(Nc2nccc(-c3ccc(OC)c(C#N)c3)n2)c1] | 1-(1-Acetylpiperidin- 4-yl)-3-(3-{[4-(3- cyano-4- methoxyphenyl) pyrimidin-2-yl] amino}- 5- methoxyphenyl)urea | ¹H NMR (DMSO-d₆) δ 9.61 (s, 1H), 8.60 (d, 1H), 8.57 (dd, 1H), 8.54 (d, 1H), 8.34 (s, 1H), 7.65 (s, 1H), 7.47 (d, 1H), 7.45-7.36 (m, 2H), 7.10 (s, 1H), 7.02 (s, 1H), 6.79 (s, 1H), 6.16 (d, 1H), 4.18-4.08 (m, 2H), 4.02 (s, 3H), 3.73 (s, 3H), 3.76-3.65 (m, 3H), 3.20-3.10 (m, 1H), 2.85-1.78 (m, 1H), 2.01 (s, 3H); LC-MS [M + H]⁺ 516.2349. |
| 345 | 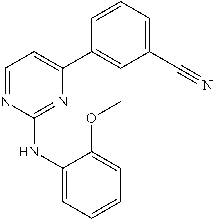 [SMILES: COc1ccccc1Nc1nccc(-c2cccc(C#N)c2)n1] | 3-{2-[(2- Methoxyphenyl) amino]pyrimidin-4- yl}benzonitrile | ¹H NMR (CDCl₃) δ 8.57-8.54 (m, 2H), 8.40-8.39 (m, 1H), 8.31-8.29 (m, 1H), 7.89 (s, 1H), 7.79-7.76 (m, 1H), 7.64-7.60 (m, 1H), 7.12 (d, 1H), 7.07-7.00 (m, 2H), 6.95-6.92 (m, 1H), 3.94 (s, 3H). LC-MS [M + H]⁺ 303.1277 |
| 346 | 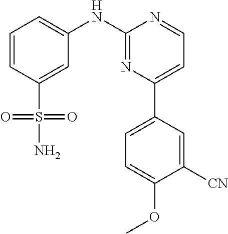 [SMILES: COc1ccc(-c2ccnc(Nc3cccc(S(N)(=O)=O)c3)n2)cc1C#N] | 3-{[4-(3-Cyano-4- methoxyphenyl) pyrimidin-2- yl]amino} benzenesulfonamide | ¹H NMR (DMSO-d₆) δ 10.07 (s, 1H), 8.62-8.58 (m, 3H), 7.85-7.83 (m, 1H), 7.57 (d, 1H), 7.55-7.48 (m, 1H), 7.44-7.39 (m, 2H), 7.33 (s, 1H), 4.02 (s, 3H). LC-MS [M + H]⁺ 382.0978 |
| 347 | 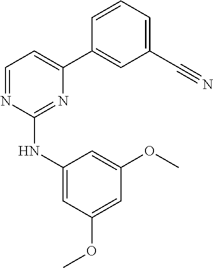 [SMILES: COc1cc(Nc2nccc(-c3cccc(C#N)c3)n2)cc(OC)c1] | 3-{2-[(3,5- Dimethoxyphenyl) amino]pyrimidin-4- yl}benzonitrile | ¹H NMR (CDCl₃) δ 8.54 (d, 1H), 8.44-8.43 (m, 1H), 8.30-8.27 (m, 1H), 7.79-7.77 (m, 1H), 7.64-7.60 (m, 1H), 7.27 (d, 1H), 7.16 (d, 1H), 6.98 (d, 2H), 6.24-6.22 (m, 1H), 3.85 (s, 6H). LC-MS [M + H]⁺ 333.1342 |
| 348 | 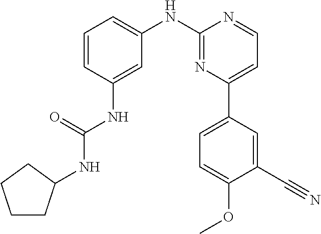 [SMILES: COc1ccc(-c2ccnc(Nc3cccc(NC(=O)NC4CCCC4)c3)n2)cc1C#N] | 1-(3-{[4-(3-Cyano-4- methoxyphenyl) pyrimidin-2- yl]amino}phenyl)-3- cyclopentylurea | ¹H NMR (DMSO-d₆) δ 9.61 (s, 1H), 8.65-8.56 (m, 2H), 8.53 (d, 1H), 8.24 (s, 1H), 8.01 (s, 1H), 7.47 (d, 1H), 7.41 (d, 1H), 7.25 (d, 1H), 7.12 (t, 1H), 6.99 (d, 1H), 6.13 (d, 1H), 4.01 (s, 3H), 4.02-3.92 (m, 1H), 1.90-1.80 (m, 2H), 1.70-1.50 (m, 4H), 1.40-1.30 (m, 2H). LC- MS [M + H]⁺ 429.2031. |
| 349 | 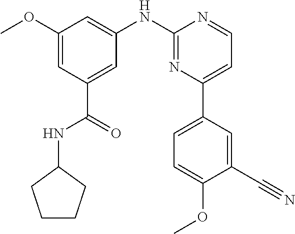 [SMILES: COc1cc(Nc2nccc(-c3ccc(OC)c(C#N)c3)n2)cc(C(=O)NC2CCCC2)c1] | 3-{[4-(3-Cyano-4- methoxyphenyl) pyrimidin-2-yl]amino}- N-cyclopentyl-5- methoxybenzamide | ¹H NMR (DMSO-d₆) δ 9.81 (s, 1H), 8.60-8.55 (m, 2H), 8.53 (dd, 1H), 8.21 (d, 1H), 7.83 (t, 1H), 7.69 (t, 1H), 7.52 (d, 1H), 7.42 (d, 1H), 6.99 (dd, 1H), 4.23 (sextet, 1H), 4.02 (s, 3H), 3.83 (s, 3H), 1.95- 1.82 (m, 2H), 1.75-1.63 (m, 2H), 1.58-1.46 (m, 4H). LC-MS [M + H]⁺ 444.2038. |
| 350 | 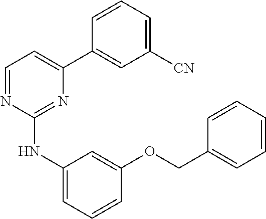 [SMILES: N#Cc1cccc(-c2ccnc(Nc3cccc(OCc4ccccc4)c3)n2)c1] | 3-(2-{[3- (Benzyloxy)phenyl] amino}pyrimidin-4- yl)benzonitrile | ¹H NMR (CDCl₃) δ 8.50 (d, 1H), 8.38-8.37 (m, 1H), 8.32-8.29 (m, 1H), 8.03 (s, 1H), 7.79-7.76 (m, 1H), 7.62-7.55 (m, 2H), 7.48-7.45 (m, 2H), 7.43-7.37 (m, 2H), 7.35- 7.22 (m, 3H), 7.15 (d, 1H), 6.73- 6.71 (m, 1H), 5.12 (s, 2H). LC-MS [M + H]⁺ 379.1614 |
| 351 | 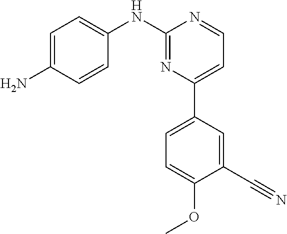 [SMILES: COc1ccc(-c2ccnc(Nc3ccc(N)cc3)n2)cc1C#N] | 5-{2-[(4- Aminophenyl)amino] pyrimidin-4-yl}-2- methoxybenzamide | ¹H NMR (DMSO-d₆) δ 9.43 (s, 1H), 8.51 (d, 1H), 8.50-8.46 (m, 2H), 8.46 (d, 1H), 7.54 (d, 2H), 7.43 (d, 1H), 7.39 (d, 1H), 6.79 (d, 1H), 4.01 (s, 3H). LC-MS [M + H]⁺ 318.1346. |
| 352 | 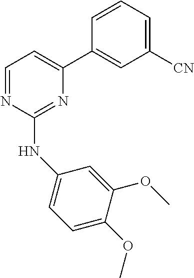 [SMILES: COc1ccc(Nc2nccc(-c3cccc(C#N)c3)n2)cc1OC] | 3-{2-[(3,4- Dimethoxyphenyl) amino]pyrimidin-4- yl}benzonitrile | ¹H NMR (CDCl₃) δ 8.52 (d, 1H), 8.41-8.40 (m, 1H), 8.28-8.26 (m, 1H), 7.79-7.76 (m, 1H), 7.63-7.59 (m, 1H), 7.48 (d, 1H), 7.26-7.23 (m, 1H), 7.12 (d, 1H), 7.06-7.04 (m, 1H), 6.89 (d, 1H), 3.95 (s, 3H), 3.90 (s, 3H). LC-MS [M + H]⁺ 333.1344 |
| 353 | 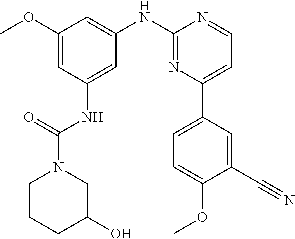 [SMILES: COc1cc(NC(=O)N2CCCC(O)C2)cc(Nc2nccc(-c3ccc(OC)c(C#N)c3)n2)c1] | N-(3-{[4-(3-Cyano- 4- methoxyphenyl) pyrimidin-2-yl] amino}- 5-methoxyphenyl)-3- hydroxypiperidine-1- carboxamide | LC-MS [M + H]⁺ 475.2094. |
| 354 | 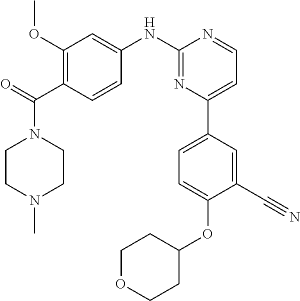 [SMILES: COc1cc(Nc2nccc(-c3ccc(OC4CCOCC4)c(C#N)c3)n2)ccc1C(=O)N1CCN(C)CC1] | 5-[2-({3-methoxy-4- [(4-methylpiperazin- 1- yl)carbonyl]phenyl} amino)pyrimidin-4- yl]-2-(tetrahydro-2H- pyran-4- yloxy)benzonitrile | ¹H NMR (DMSO-d₆) δ 9.99 (s, 1H), 9.87 (br s, 1H), 8.62 (d, 1H), 8.60 (d, 1H), 8.46 (dd, 1H), 7.93 (br s, 1H), 7.57-7.54 (m, 2H), 7.38-7.32 (m, 1H), 7.20 (d, 1H), 4.99-4.93 (m, 1H), 4.64-4.58 (m, 1H), 3.90- 3.85 (m, 4H), 3.50-3.16 (br m, 4H), 3.12-2.94 (m, 4H), 2.86 (s, 3H), 2.06-2.02 (m, 2H), 1.73-1.65 (m, 2H). LC-MS [M + H]⁺ 529.2547 |
| 355 | 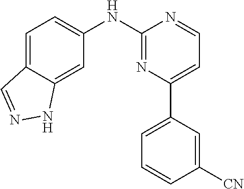 [SMILES: N#Cc1cccc(-c2ccnc(Nc3ccc4cn[nH]c4c3)n2)c1] | 3-[2-(1H-Indazol-6- ylamino)pyrimidin- 4-yl]benzonitrile | ¹H NMR (CDCl₃) δ 8.91 (d, 1H), 8.56 (s, 1H), 8.47 (d, 1H), 8.17 (s, 1H), 8.06 (d, 1H), 7.88 (d, 1H), 7.76-7.72 (m, 1H), 7.60-7.58 (m, 2H), 6.80-6.77 (m, 1H). LC-MS [M + H]⁺ 313.1192 |
| 356 | 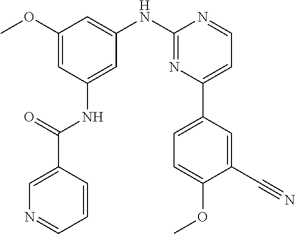 [SMILES: COc1cc(NC(=O)c2cccnc2)cc(Nc2nccc(-c3ccc(OC)c(C#N)c3)n2)c1] | N-(3-{[4-(3-Cyano- 4- methoxyphenyl) pyrimidin-2-yl] amino}-5- methoxyphenyl) pyridine-3- carboxamide | ¹H NMR (DMSO-d₆) δ 10.44 (s, 1H), 9.78 (s, 1H), 9.13 (d, 1H), 8.77 (dd, 1H), 8.66 (d, 1H), 8.60- 8.55 (m, 2H), 8.34 (dt, 1H), 8.06 (s, 1H), 7.58 (dd, 1H), 7.51 (d, 1H), 7.42 (d, 1H), 7.26 (s, 1H), 7.02 (s, 1H), 4.01 (s, 3H), 3.78 (s, 3H); LC-MS [M + H]⁺ 453.1670. |
| 357 | 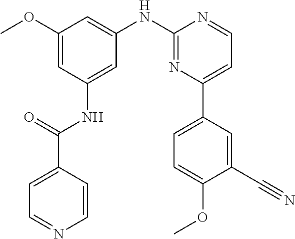 [SMILES: COc1cc(NC(=O)c2ccncc2)cc(Nc2nccc(-c3ccc(OC)c(C#N)c3)n2)c1] | N-(3-{[4-(3-Cyano- 4- methoxyphenyl) pyrimidin-2-yl] amino}-5- methoxyphenyl) pyridine-4- carboxamide] | ¹H NMR (DMSO-d₆) δ 10.50 (s, 1H), 9.79 (s, 1H), 8.81-8.77 (m, 2H), 8.66 (d, 1H), 8.59-8.54 (m, 2H), 8.06 (s, 1H), 7.93-7.88 (m, 2H), 7.51 (d, 1H), 7.42 (d, 1H), 7.27 (s, 1H), 7.03 (s, 1H), 4.01 (s, 3H), 3.78 (s, 3H). LC-MS [M + H]⁺ 453.1670 |
| 358 | 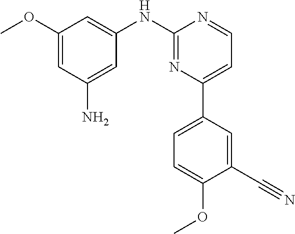 [SMILES: COc1cc(N)cc(Nc2nccc(-c3ccc(OC)c(C#N)c3)n2)c1] | 5-{2-[(3-Amino-5- methoxyphenyl) amino]pyrimidin- 4-yl}-2- methoxybenzonitrile | ¹H NMR (DMSO-d₆) δ 9.41 (s, 1H), 8.56 (d, 1H), 8.54-8.46 (m, 2H), 7.44 (d, 1H), 7.43 (d, 1H), 6.81 (s, 1H), 6.62 (t, 1H), 5.83 (t, 1H), 5.07 (br s, 2H), 4.01 (s, 3H), 3.68 (s, 3H). LC-MS [M + H]⁺ 348.1449. |
| 359 | 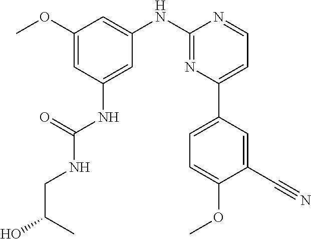 [SMILES: COc1cc(NC(=O)NC[C@H](C)O)cc(Nc2nccc(-c3ccc(OC)c(C#N)c3)n2)c1] | 1-(3-{[4-(3-Cyano-4- methoxyphenyl) pyrimidin-2-yl] amino}- 5-methoxyphenyl)-3- [(2S)-2- hydroxypropyl]urea | ¹H NMR (DMSO-d₆) δ 9.61 (s, 1H), 8.62-8.50 (m, 4H), 7.47 (d, 1H), 7.45-7.36 (m, 2H), 7.11 (t, 1H), 6.80 (s, 1H), 6.18 (t, 1H), 4.77 (d, 1H) 4.02 (s, 3H), 3.73 (s, 3H), 3.70-3.64 (m, 1H), 3.20-3.11 (m, 1H), 3.00-2.90 (m, 1H), 1.06 (d, 1H). LC-MS [M + H]⁺ 449.1937. |
| 360 | 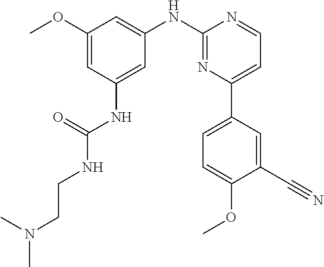 [SMILES: COc1cc(NC(=O)NCCN(C)C)cc(Nc2nccc(-c3ccc(OC)c(C#N)c3)n2)c1] | 1-(3-{[4-(3-Cyano-4- methoxyphenyl) pyrimidin-2-yl] amino}- 5-methoxyphenyl)-3- [2- (dimethylamino) ethyl]urea | ¹H NMR (DMSO-d₆) δ 9.63 (s, 1H), 8.61 (d, 1H), 8.57-8.54 (m, 2H), 7.52 (s, 1H), 7.48 (d, 1H), 7.43 (s, 1H), 7.42 (d, 1H), 7.13 (s, 1H), 6.83 (s, 1H), 6.54 (t, 1H), 4.02 (s, 3H), 3.73 (s, 3H), 3.51-3.42 (m, 2H), 3.20-3.10 (m, 2H), 2.82 (m, 6H); LC-MS [M + H]⁺ 462.2235. |
| 361 | 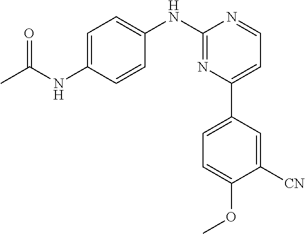 [SMILES: COc1ccc(-c2ccnc(Nc3ccc(NC(C)=O)cc3)n2)cc1C#N] | N-(4-{[4-(3-Cyano- 4- methoxyphenyl) pyrimidin-2- yl]amino}phenyl) acetamide | ¹H NMR (CDCl₃) δ 8.42 (d, 1H), 8.31-8.28 (m, 2H), 7.64-7.60 (m, 2H), 7.55-7.52 (m, 2H), 7.15-7.07 (m, 2H), 4.03 (s, 3H), 2.16 (s, 3H), LC-MS [M + H]⁺ 360.1448 |
| 362 | 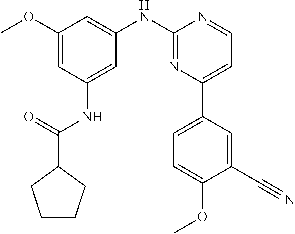 [SMILES: COc1cc(NC(=O)C2CCCC2)cc(Nc2nccc(-c3ccc(OC)c(C#N)c3)n2)c1] | N-(3-{[4-(3-Cyano- 4- methoxyphenyl) pyrimidin-2-yl] amino}-5- methoxyphenyl) cyclopentane- carboxamide | ¹H NMR (DMSO-d₆) δ 9.84 (s, 1H), 9.68 (s, 1H), 8.62-8.55 (m, 2H), 8.56 (d, 1H), 7.80 (s, 1H), 7.49 (d, 1H), 7.40 (d, 1H), 7.15 (s, 1H), 6.91 (s, 1H), 4.01 (s, 3H), 3.74 (s, 3H), 2.85-2.72 (m, 1H), 1.90-1.890 (m, 2H), 1.80-1.60 (m, 4H), 1.60- 1.50 (m, 2H). LC-MS [M + H]⁺ 444.2028. |
| 363 | 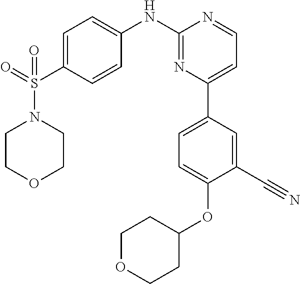 [SMILES: N#Cc1cc(-c2ccnc(Nc3ccc(S(=O)(=O)N4CCOCC4)cc3)n2)ccc1OC1CCOCC1] | 5-(2-{[4-(morpholin- 4- ylsulfonyl)phenyl] amino}pyrimidin- 4-yl)- 2-(tetrahydro-2H- pyran-4- yloxy)benzonitrile | ¹H NMR (DMSO-d₆) δ 10.3 (s, 1H), 8.65 (d, 1H), 8.59 (d, 1H), 8.50 (dd, 1H), 8.12 (d, 2H), 7.69 (d, 2H), 7.62 (d, 1H), 7.58 (d, 1H), 4.99-4.93 (m, 1H), 3.90-3.86 (m, 2H), 3.63 (t, 4H), 3.59-3.53 (m, 2H), 2.85 (t, 4H), 2.10-2.01 (m, 2H), 1.76-1.66 (m, 2H). LC-MS [M + H]⁺ 522.1801 |
| 364 | 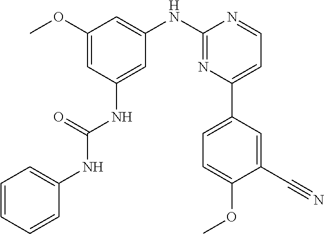 [SMILES: COc1cc(NC(=O)Nc2ccccc2)cc(Nc2nccc(-c3ccc(OC)c(C#N)c3)n2)c1] | 1-(3-{[4-(3-Cyano-4- methoxyphenyl) pyrimidin-2-yl] amino}- 5-methoxyphenyl)-3- phenylurea | ¹H NMR (DMSO-d₆) δ 9.69 (s, 1H), 8.69-8.60 (m, 3H), 8.59 (d, 1H), 8.56 (d, 1H), 7.52-7.45 (m, 4H), 7.39 (d, 1H), 7.29 (t, 2H), 7.19 (t, 1H), 6.97 (t, 1H), 6.84 (s, 1H), 3.99 (s, 3H), 3.76 (s, 3H). LC-MS [M + H]⁺ 467.1843. |
| 365 | 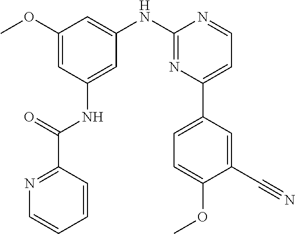 [SMILES: COc1cc(NC(=O)c2ccccn2)cc(Nc2nccc(-c3ccc(OC)c(C#N)c3)n2)c1] | N-(3-{[4-(3-Cyano- 4- methoxyphenyl) pyrimidin-2-yl] amino}- 5- methoxyphenyl) pyrimidin-2- carboxamide | ¹H NMR (DMSO-d₆) δ 10.49 (s, 1H), 9.75 (s, 1H), 8.78-8.72 (m, 1H), 6.67 (d, 1H), 8.62-8.54 (m, 2H), 8.27-8.20 (m, 2H), 8.08 (dt, 1H), 7.69 (ddd, 1H), 7.51 (d, 1H), 7.42 (d, 1H), 7.24 (t, 1H), 7.10 (t, 1H), 4.02 (s, 3H), 3.79 (s, 3H). LC-MS [M + H]⁺ 453.1519. |
| 366 | 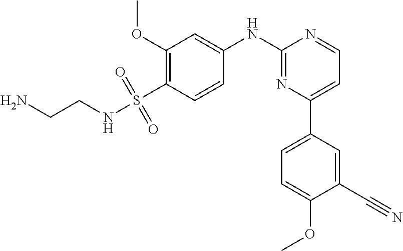 [SMILES: COc1ccc(-c2ccnc(Nc3ccc(S(=O)(=O)NCCN)c(OC)c3)n2)cc1C#N] | N-(2-Aminoethyl)-4- {[4-(3-cyano-4- methoxyphenyl) pyrimidin-2-yl]amino}- 2- methoxybenzene- sulfonamide | ¹H NMR (DMSO-d₆) δ 10.22 (s, 1 H), 8.66 (d, 1 H), 8.61 (d, 1 H), 8.53 (dd, 1 H), 8.03 (d, 1 H), 7.77 (br. s., 4 H), 7.66 (d, 1 H), 7.62 (d, 1 H), 7.46 (d, 1 H), 7.42 (dd, 1 H), 7.31 (t, 1 H), 4.03 (s, 3 H), 3.97 (s, 3 H), 2.92-2.99 (m, 2 H), 2.81- 2.90 (m, 2 H). LC-MS [M + H]⁺ 455.1496 |
| 367 | 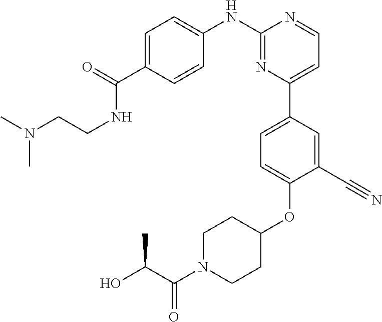 [SMILES: C[C@H](O)C(=O)N1CCC(Oc2ccc(-c3ccnc(Nc4ccc(C(=O)NCCN(C)C)cc4)n3)cc2C#N)CC1] | 4-({4-[3-cyano-4- ({1-[(2S)-2- hydroxypropanoyl] piperidin-4- yl}oxy)phenyl] pyrimidin-2-yl} amino)-N-[2- (dimethylamino) ethyl]benzamide | ¹H NMR (DMSO-d₆) δ 10.1 (s, 1H), 9.28 (br s, 1H), 8.65 (d, 1H), 8.58- 8.55 (m, 1H), 8.48 (dd, 1H), 7.93 (d, 2H), 7.85 (d, 2H), 7.60-7.57 (m, 2H), 5.00-4.94 (m, 1H), 4.48- 4.44 (m, 1H), 3.82-3.78 (m, 2H), 3.60-3.52 (m, 2H), 3.30-3.25 (m, 4H), 2.50 (s, 6H), 2.09-2.02 (m, 2H), 1.82-1.65 (m, 2H), 1.21 (d, 3H). LC-MS [M + H]⁺ 558.2823 |
| 368 | 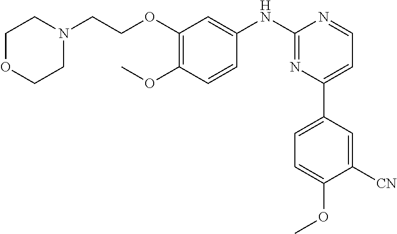 [SMILES: COc1ccc(-c2ccnc(Nc3ccc(OC)c(OCCN4CCOCC4)c3)n2)cc1C#N] | 2-Methoxy-5-[2-({4- methoxy-3-[2- (morpholin-4- yl)ethoxy]phenyl} amino)pyrimidin-4- yl]benzonitrile | ¹H NMR (CDCl₃-) δ 8.44 (d, 1H), 8.33 (d, 1H), 8.25-8.23 (m, 1H), 7.41 (d, 1H), 7.10-7.03 (m, 4H), 6.89 (d, 1H), 4.23-4.20 (m, 2H), 4.01 (s, 3H), 3.87 (s, 3H), 3.73- 3.71 (m, 4H), 2.90-2.87 (m, 2H), 2.62-2.59 (m, 4H). LC-MS [M + H]⁺ 462.2140 |
| 369 | 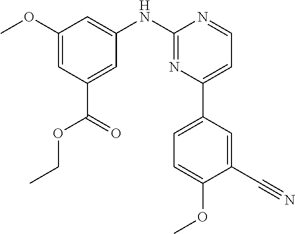 [SMILES: CCOC(=O)c1cc(Nc2nccc(-c3ccc(OC)c(C#N)c3)n2)cc(OC)c1] | Ethyl 2-{[4-(3- cyano-4- methoxyphenyl) pyrimidin-2-yl] amino}- 5-methoxybenzoate | ¹H NMR (DMSO-d₆) δ 9.95 (s, 1H), 8.60 (d, 1H), 8.58 (d, 1H), 8.52 (dd, 1H), 8.16 (s, 1H), 7.78 (t, 1H), 7.54 (d, 1H), 7.44 (d, 1H), 7.09 (dd, 1H), 4.34 (q, 2H), 4.02 (s, 3H), 3.83 (s, 3H), 1.32 (t, 3H). ). LC- MS [M + H]⁺ 405.1566. |
| 370 | 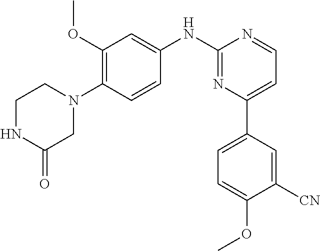 [SMILES: COc1ccc(-c2ccnc(Nc3ccc(N4CCNC(=O)C4)c(OC)c3)n2)cc1C#N] | 2-Methoxy-5-(2-{[3- methoxy-4-(3- oxopiperazin-1- yl)phenyl]amino} pyrimidin-4- yl)benzonitrile | ¹H NMR (CDCl₃) δ 8.44 (d, 1H), 8.37 (d, 1H), 8.29-8.27 (m, 1H), 7.59 (d, 1H), 7.14-7.09 (m, 3H), 6.93 (d, 1H), 4.03 (s, 3H), 3.97 (s, 3H), 3.78 (s, 2H), 3.51-3.48 (m, 2H), 3.34-3.31 (m, 2H). LC-MS [M + H]⁺ 431.2048 |
| 371 | 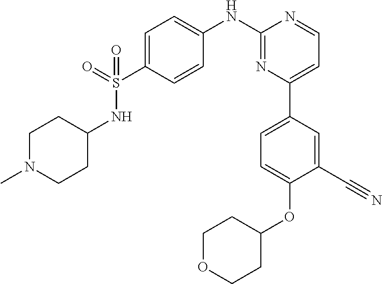 [SMILES: CN1CCC(NS(=O)(=O)c2ccc(Nc3nccc(-c4ccc(OC5CCOCC5)c(C#N)c4)n3)cc2)CC1] | 4-({4-[3-cyano-4- (tetrahydro-2H- pyran-4- yloxy)phenyl] pyrimidin-2-yl} amino)-N- (1-methylpiperidin- 4- yl)benzenesulfonamide | ¹H NMR (DMSO-d₆) δ 10.2 (s, 1H), 9.15 (br s, 1H), 8.65 (d, 1H), 8.58 (d, 1H), 8.48 (dd, 12H), 8.06-8.02 (m, 2H), 7.82-7.75 (m, 3H), 7.61 (d, 1H), 7.57 (d, 1H), 5.00-4.94 (m, 1H), 3.91-3.85 (m, 2H), 3.59-3.53 (m, 2H), 3.35-3.29 (m, 2H), 3.20- 3.16 (m, 1H), 2.95-2.89 (m, 2H), 2.67 (d, 3H), 2.09-2.02 (m, 2H), 1.82-1.65 (m, 4H), 1.60-1.53 (m, 2H). LC-MS [M + H]⁺ 549.2292 |
| 372 | 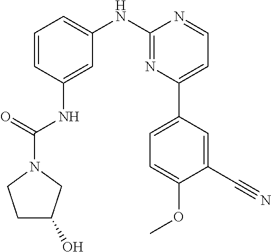 [SMILES: COc1ccc(-c2ccnc(Nc3cccc(NC(=O)N4CC[C@@H](O)C4)c3)n2)cc1C#N] | (3R)-N-(3-{[4-(3- Cyano-4- methoxyphenyl) pyrimidin-2- yl]amino}phenyl)-3- hydroxypyrrolidine- 1-carboxamide | ¹H NMR (DMSO-d₆) δ 9.63 (s, 1H), 8.66-8.58 (m, 2H), 8.54 (d, 1H), 8.23 (s, 1H), 8.11 (s, 1H), 7.47 (d, 1H), 7.40 (d, 1H), 7.23 (d, 1H), 7.14 (t, 1H), 7.04 (d, 1H), 4.31 (br s, 1H), 4.01 (s, 3H), 3.55-3.40 (m, 3H), 3.33 (d, 1H), 2.00-1.86 (m, 1H), 1.86-1.64 (m, 1H). LC-MS [M + H]⁺ 431.1823. |
| 373 | 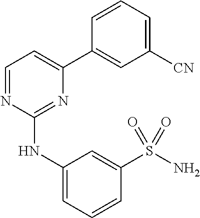 [SMILES: N#Cc1cccc(-c2ccnc(Nc3cccc(S(N)(=O)=O)c3)n2)c1] | 3-{[4-(3- Cyanophenyl) pyrimidin-2- yl]amino} benzenesulfonamide] | ¹H NMR (CD-3OD) δ 8.73-8.72 (m, 1H), 8.58-8.55 (m, 3H), 7.89-7.86 (m, 1H), 7.80-7.77 (m, 1H), 7.74- 7.70 (m, 1H), 7.55-7.50 (m, 3H). LC-MS [M + H]⁺ 352.0863 |
| 374 | 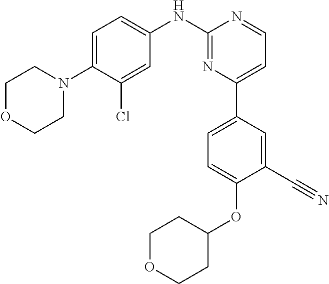 [SMILES: N#Cc1cc(-c2ccnc(Nc3ccc(N4CCOCC4)c(Cl)c3)n2)ccc1OC1CCOCC1] | 5-(2-{[3-chloro-4- (morpholin-4- yl)phenyl]amino} pyrimidin-4-yl)-2- (tetrahydro-2H- pyran-4- yloxy)benzonitrile | ¹H NMR (DMSO-d₆) δ 9.78 (s, 1H), 8.54-8.58 (m, 2H), 8.43 (dd, 1H), 8.05 (d, 1H), 7.65 (dd, 1H), 7.55 (d, 1H), 7.49 (d, 1H), 7.15 (d, 1H), 4.95 (hep, 1H), 3.91-3.85 (m, 2H), 3.74 (t, 4H), 3.6-3.5 (m, 2H), 2.93 (t, 4H), 2.1-2.0 (m, 2H), 1.64-1.74 (m, 2H); LC-MS [M + H]⁺ 492.1760 |
| 375 | 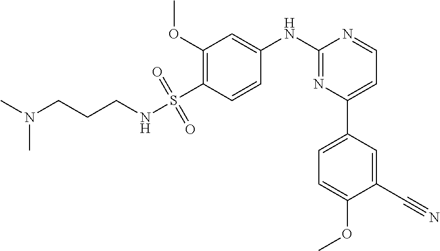 [SMILES: COc1ccc(-c2ccnc(Nc3ccc(S(=O)(=O)NCCCN(C)C)c(OC)c3)n2)cc1C#N] | 4-{[4-(3-Cyano-4- methoxyphenyl) pyrimidin-2-yl]amino}- N-[3- (dimethylamino) propyl]-2- methoxybenzene- sulfonamide | ¹H NMR (DMSO-d₆) δ 10.19 (s, 1 H), 8.66 (d, 1 H), 8.61 (d, 1 H), 8.53 (dd, 1 H), 8.02 (d, 1 H), 7.65 (d, 1 H), 7.61 (d, 1 H), 7.46 (d, 1 H), 7.40 (dd, 1 H), 7.22 (t, 1 H), 4.03 (s, 3 H), 3.97 (s, 3 H), 3.01- 3.09 (m, 2 H), 2.80 (q, 2 H), 2.75 (s, 3 H), 2.74 (s, 3 H), 1.70-1.81 (m, 2 H). LC-MS [M + H]⁺ 497.1966 |
| 376 | 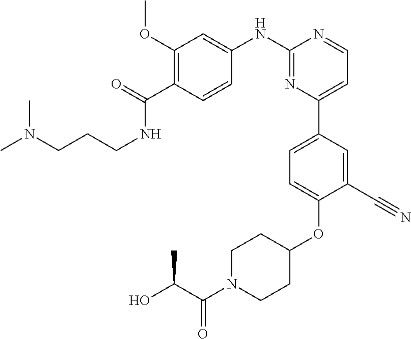 [SMILES: COc1cc(Nc2nccc(-c3ccc(OC4CCN(C(=O)[C@H](C)O)CC4)c(C#N)c3)n2)ccc1C(=O)NCCCN(C)C] | 4-({4-[3-cyano-4- ({1-[(2S)-2- hydroxypropanoyl] piperidin-4- yl}oxy)phenyl] pyrimidin-2-yl} amino)-N-[3- (dimethylamino) propyl]-2- methoxybenzamide | ¹H NMR (DMSO-d₆) δ 10.1 (s, 1H), 9.33 (br s, 1H), 8.64 (d, 1H), 8.62 (d, 1H), 8.48 (dd, 1H), 8.30 (t, 1H), 7.97 (s, 1H), 7.82 (d, 1H), 7.59- 7.57 (m, 2H), 7.37 (dd, 1H), 5.03 (br s, 2H), 4.50-4.45 (m, 1H), 3.99 (s, 3H), 3.85-3.65 (m, 2H), 3.60- 3.46 (m, 2H), 3.31-3.25 (m, 2H), 3.10-3.03 (m, 2H), 2.79 (s, 6H), 2.09-1.94 (m, 2H), 1.92-1.87 (m, 2H), 1.79-1.60 (m, 2H), 1.20 (d, 3H). LC-MS [M + H]⁺ 602.3085 |
| 377 | 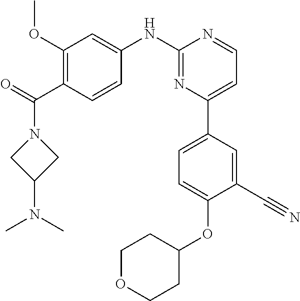 [SMILES: COc1cc(Nc2nccc(-c3ccc(OC4CCOCC4)c(C#N)c3)n2)ccc1C(=O)N1CC(N(C)C)C1] | 5-{2-[(4-{[3- (dimethylamino) azetidin-1-yl]carbonyl}- 3- methoxyphenyl) amino]pyrimidin- 4-yl}-2- (tetrahydro-2H- pyran-4- yloxy)benzonitrile | ¹H NMR (DMSO-d₆) δ 10.3 (br s, 1H), 10.0 (s, 1H), 8.62 (d, 1H), 8.60 (d, 1H), 8.46 (dd, 1H), 7.91 (s, 1H), 7.57-7.55 (m, 2H), 7.34 (s, 2H), 4.99-4.94 (m, 1H), 4.24-4.14 (m, 3H), 4.12-4.05 (m, 2H), 3.91 (s, 3H), 3.90-3.85 (m, 2H), 3.59-3.53 (m, 2H), 3.12-2.94 (m, 4H), 2.80 (s, 3H), 2.74 (s, 3H), 2.08-2.02 (m, 2H), 1.74-1.65 (m, 2H). LC-MS [M + H]⁺ 529.2549 |
| 378 | 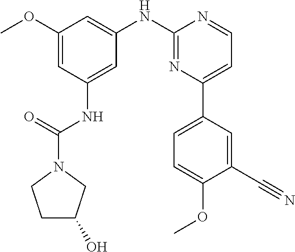 [SMILES: COc1cc(NC(=O)N2CC[C@@H](O)C2)cc(Nc2nccc(-c3ccc(OC)c(C#N)c3)n2)c1] | (3R)-N-(3-{[4-(3- Cyano-4- methoxyphenyl) pyrimidin-2-yl]amino}- 5-methoxyphenyl)-3- hydroxypyrrolidine- 1-carboxamide | ¹H NMR (DMSO-d₆) δ 9.59 (s, 1H), 8.64-8.56 (m, 2H), 8.54 (d, 1H), 8.07 (s, 1H), 7.75 (s, 1H), 7.46 (d, 1H), 7.40 (d, 1H), 7.06 (t, 1H), 6.77 (t, 1H), 4.34-4.26 (m, 1H), 4.01 (s, 3H), 3.73 (s, 3H), 3.50- 3.42 (m, 3H), 3.31 (d, 1H), 2.0-1.86 (m, 1H), 1.86-1.75 (m, 1H); LC- MS [M + H]⁺ 461.1946 |
| 379 | 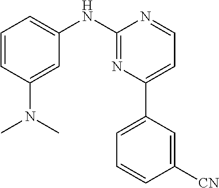 [SMILES: CN(C)c1cccc(Nc2nccc(-c3cccc(C#N)c3)n2)c1] | 3-(2-{[3- (Dimethylamino) phenyl]amino} pyrimidin- 4-yl)benzonitrile | ¹H NMR (CDCl₃) δ 8.52 (d, 1H), 8.46-8.45 (m, 1H), 8.33-8.30 (m, 1H), 7.82-7.80 (m, 1H), 7.67-7.63 (m, 1H), 7.61-7.59 (m, 1H), 7.31 (d, 1H), 7.21-7.17 (m, 2H), 6.73- 6.70 (m, 1H), 3.09 (s, 6H). LC-MS [M + H]⁺ 316.1542 |
| 380 | 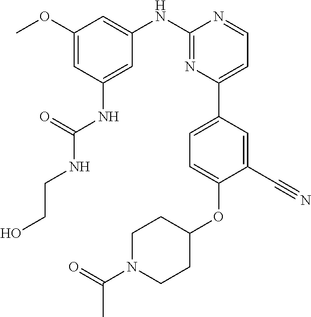 [SMILES: COc1cc(NC(=O)NCCO)cc(Nc2nccc(-c3ccc(OC4CCN(C(C)=O)CC4)c(C#N)c3)n2)c1] | 1-{3-[(4-{4-[(1- Acetylpiperidin-4- yl)oxy]-4- cyanophenyl} pyrimidin- 2-yl)amino]-5- methoxyphenyl}-3- (2-hydroxyethyl)urea | ¹H NMR (DMSO-d₆) δ 9.61 (s, 1H), 8.62-8.58 (m, 1H), 8.57-8.51 (m, 3H), 7.53 (d, 1H), 7.48 (d, 1H), 7.43 (s, 1H), 7.10 (t, 1H), 6.78 (s, 1H), 6.19 (br s, 1H), 5.00 (sept. 1H), 3.73 (s, 3H), 3.80-3.60 (m, 3H), 3.48-3.38 (m, 5H), 3.21-3.10 (m, 2H), 2.04 (s, 3H), 1.90-1.83 (m, 1H), 1.80-1.70 (m, 1H), 1.70-1.60 (m, 1H). LC-MS [M + H]⁺ 546.2459. |
| 381 | 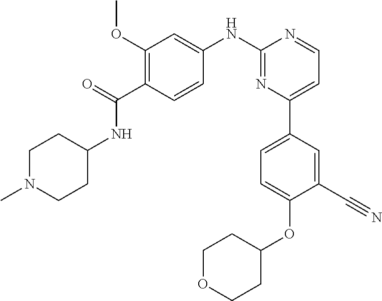 [SMILES: COc1cc(Nc2nccc(-c3ccc(OC4CCOCC4)c(C#N)c3)n2)ccc1C(=O)NC1CCN(C)CC1] | 4-({4-[3-cyano-4- (tetrahydro-2H- pyran-4- yloxy)phenyl] pyrimidin-2-yl} amino)-2- methoxy-N-(1- methylpiperidin-4- yl)benzamide | ¹H NMR (DMSO-d₆) δ 10.0 (s, 1H), 9.28 (br s, 1H), 8.63 (d, 1H), 8.61 (d, 1H), 8.47 (dd, 1H), 7.97 (d, 1H), 7.94 (d, 1H), 7.71 (dd, 1H), 7.58 (s, 1H), 7.56 (d, 1H), 7.37 (dd, 1H), 5.00-4.94 (m, 1H), 3.96 (s, 3H), 3.92-3.86 (m, 2H), 3.59-3.53 (m, 2H), 3.47 (d, 2H), 3.40-3.35 (m, 1H), 3.14-3.06 (m, m2H), 3.14- 3.06 (m, 2H), 2.78 (d, 3H), 2.10- 2.02 (m, 4H), 1.77-1.65 (m, 4H). LC-MS [M + H]⁺ 543.2697 |
| 382 | 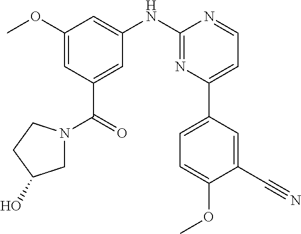 [SMILES: COc1cc(Nc2nccc(-c3ccc(OC)c(C#N)c3)n2)cc(C(=O)N2CC[C@@H](O)C2)c1] | 5-{2-[(3-{[(3R)-3- Hydroxypyrrolidin- 1-yl]carbonyl}-5- methoxyphenyl) amino]pyrimidin- 4-yl}-2- methoxybenzonitrile | LC-MS [M + H]⁺ 446.1828. |
| 383 | 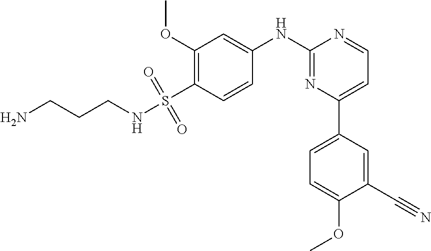 [SMILES: COc1ccc(-c2ccnc(Nc3ccc(S(=O)(=O)NCCCN)c(OC)c3)n2)cc1C#N] | N-(3-Aminopropyl)- 4-{[4-(3-cyano-4- methoxyphenyl) pyrimidin-2-yl]amino}- 2- methoxybenzene- sulfonamide | ¹H NMR (DMSO-d₆) δ 10.18 (s, 1 H), 8.65 (d, 1 H), 8.61 (d, 1 H), 8.53 (dd, 1 H), 8.01 (d, 2 H), 7.59- 7.68 (m, 4 H), 7.46 (dd, 1 H), 7.40 (dd, 1 H), 7.22 (t, 1 H), 4.03 (s, 3 H), 3.94-3.99 (m, 3 H), 2.76- 2.85 (m, 4 H), 1.62-1.72 (m, 2 H). LC-MS [M + H]⁺ 469.1653 |
| 384 | 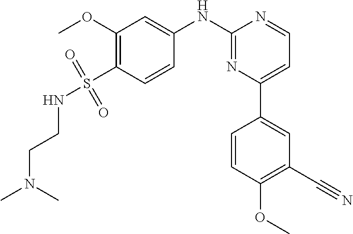 [SMILES: COc1ccc(-c2ccnc(Nc3ccc(S(=O)(=O)NCCN(C)C)c(OC)c3)n2)cc1C#N] | 4-{[4-(3-Cyano-4- methoxyphenyl) pyrimidin-2-yl]amino}- N-[2- (dimethylamino) ethyl]-2- methoxybenzene- sulfonamide] | ¹H NMR (DMSO-d₆) δ 10.16 (s, 1 H), 8.65 (d, 1 H), 8.61 (d, 1 H), 8.53 (dd, 1 H), 7.98 (d, 1 H), 7.58- 7.66 (m, 2 H), 7.47 (d, 1 H), 7.40 (dd, 1 H), 6.95 (t, 1 H), 4.02 (s, 3 H), 3.95 (s, 3 H), 3.36 (t, 2 H), 2.70- 2.89 (m, 2 H), 1.40-1.61 (m, 2 H). LC-MS [M + H]⁺ 483.1814 |
| 385 | 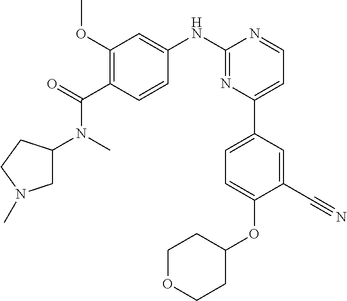 [SMILES: COc1cc(Nc2nccc(-c3ccc(OC4CCOCC4)c(C#N)c3)n2)ccc1C(=O)N(C)C1CCN(C)C1] | 4-({4-[3-cyano-4- (tetrahydro-2H- pyran-4- yloxy)phenyl] pyrimidin-2-yl} amino)-2- methoxy-N-methyl- N-(1- methylpyrrolidin-3- yl)benzamide | ¹H NMR (DMSO-d₆) δ 9.96 (s, 1H), 8.62-8.60 (m, 2H), 8.46 (dd, 1H), 7.89 (br s, 1H), 7.57-7.54 (m, 2H), 7.33 (d, 1H), 7.13 (t, 1H), 4.99- 4.93 (m, 1H), 3.87 (s, 3H), 4.04- 3.85 (m, 5H), 3.59-3.53 (m, 2H), 2.94-2.67 (m, 7H), 2.49 (s, 6H), 2.09-2.03 (m, 2H), 1.73-1.64 (m, 2H). LC-MS [M + H]⁺ 543.2699 |
| 386 | 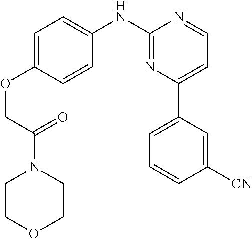 [SMILES: N#Cc1cccc(-c2ccnc(Nc3ccc(OCC(=O)N4CCOCC4)cc3)n2)c1] | 3-[2-({4-[2- (Morpholin-4-yl)-2- oxoethoxy]phenyl} amino)pyrimidin-4- yl]benzonitrile | ¹H NMR (CDCl₃) δ 8.48 (d, 1H), 8.36-8.35 (m, 1H), 8.30-8.27 (m, 1H), 7.81-7.88 (m, 1H), 7.66-7.58 (m, 3H), 7.14 (d, 1H), 7.00-6.97 (m, 2H), 4.72 (s, 2H), 3.71-3.69 (m, 4H), 3.66-3.63 (m, 4H). LC-MS [M + H]⁺ 416.1719 |
| 387 | 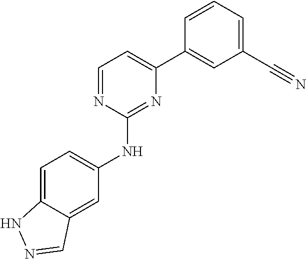 [SMILES: N#Cc1cccc(-c2ccnc(Nc3ccc4[nH]ncc4c3)n2)c1] | 3-[2-(1H-Indazol-5- ylamino)pyrimidin- 4-yl]benzonitrile | ¹H NMR (CDCl₃) δ 8.51 (d, 1H), 8.40 (s, 1H), 8.31 (d, 1H), 8.14 (s, 1H), 8.03 (s, 1H), 7.80 (d, 1H), 7.67-7.64 (m, 1H), 7.58-7.53 (m, 2H), 7.17 (d, 1H). LC-MS [M + H]⁺ 313.1201 |
| 388 | 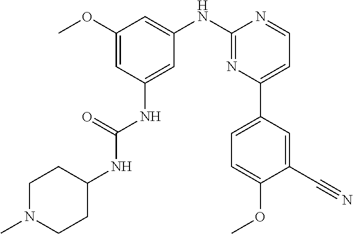 [SMILES: COc1cc(NC(=O)NC2CCN(C)CC2)cc(Nc2nccc(-c3ccc(OC)c(C#N)c3)n2)c1] | 1-(3-{[4-(3-Cyano-4- methoxyphenyl) pyrimidin-2-yl]amino}- 5-methoxyphenyl)-3- (1-methylpiperazin- 4-yl)urea | LC-MS [M + H]⁺ 488.2388. |
| 389 | 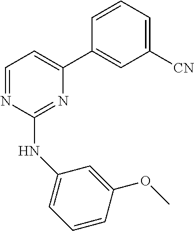 [SMILES: COc1cccc(Nc2nccc(-c3cccc(C#N)c3)n2)c1] | 3-{2-[(3- Methoxyphenyl)amino] pyrimidin-4- yl}benzonitrile | ¹H NMR (CDCl₃) δ 8.54 (d, 1H), 8.41-8.40 (m, 1H), 8.30-8.27 (m, 1H), 7.79-7.77 (m, 1H), 7.76-7.60 (m, 1H), 7.54-7.52 (m, 1H), 7.33 (s, 1H), 7.29-7.25 (m, 1H), 7.16-7.11 (m, 2H), 6.66-6.63 (m, 1H), 3.87 (s, 3H). LC-MS [M + H]⁺ 303.1244 |
| 390 | 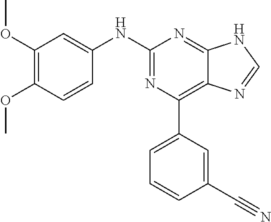 [SMILES: COc1ccc(Nc2nc(-c3cccc(C#N)c3)c3nc[nH]c3n2)cc1OC] | 3-{2-[(3,4- Dimethoxyphenyl) amino]-3H-purin-6- yl}benzonitrile | ¹H NMR (DMSO-d₆) δ 9.47 (br. s., 1 H), 9.05-9.15 (m, 2 H), 8.43 (s, 1 H), 8.02-8.08 (m, 1 H), 7.84 (t, 1 H), 7.68 (d, 1 H), 7.27 (dd, 1 H), 6.92 (d, 1 H), 3.81 (s, 3 H), 3.74 (s, 3 H). |
| 391 | 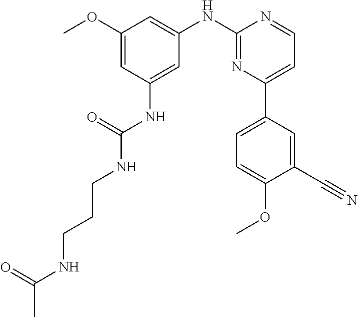 [SMILES: COc1cc(NC(=O)NCCCNC(C)=O)cc(Nc2nccc(-c3ccc(OC)c(C#N)c3)n2)c1] | N-(3-{[(3-{[4-(3- Cyano-4- methoxyphenyl) pyrimidin-2-yl]amino}- 5- methoxyphenyl) carbamoyl] amino}propyl) acetamide | LC-MS [M + H]⁺ 490.2197. |
| 392 | 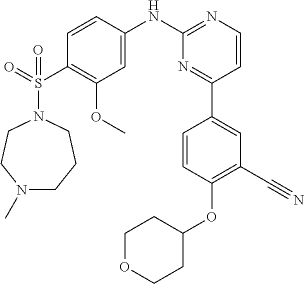 [SMILES: COc1cc(Nc2nccc(-c3ccc(OC4CCOCC4)c(C#N)c3)n2)ccc1S(=O)(=O)N1CCCN(C)CC1] | 5-[2-({3-Methoxy-4- [(4-methyl-1,4- diazepan-1- yl)sulfonyl]phenyl} amino)pyrimidin-4- yl]-2-(tetrahydro-2H- pyran-4- yloxy)benzonitrile | ¹H NMR (DMSO d-6) δ 10.18 (s, 1H), 8.65 (d, 1H), 8.60 (s, 1H), 8.47 (d, 1H), 7.96 (s, 1H), 7.67- 7.55 (m, 3H), 7.41 (d, 1H), 4.98- 4.92 (m, 1H), 3.93 (s, 3H), 3.91- 3.85 (m, 2H), 3.59-3.53 (m, 2H), 3.40-3.27 (m, 5H), 2.25 (s, 3H), 2.07-2.03 (m, 2H), 1.79-1.65 (m, 4H). LC-MS [M + H]⁺ 579.2411 |
| 393 | 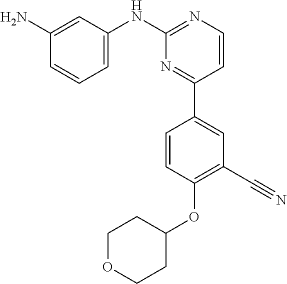 [SMILES: N#Cc1cc(-c2ccnc(Nc3cccc(N)c3)n2)ccc1OC1CCOCC1] | 5-{2-[(3- Aminophenyl)amino] pyrimidin-4-yl}-2- (tetrahydro-2H- pyran-4- yloxy)benzonitrile | ¹H NMR (DMSO d-6) δ 9.88 (s, 1H), 8.59-8.55 (m, 2H), 8.47 (d, 1H), 7.76 (br s, 1H), 7.56-7.52 (m, 3H), 7.30 (t, 1H), 6.75, 4.98-4.94 (m, 1H), 3.91-3.85 (m, 2H), 3.59- 3.54 (m, 2H), 2.07-2.02 (m, 2H), 1.74-1.65 (m, 2H). LC-MS [M + H]⁺ 388.1877 |
| 394 | 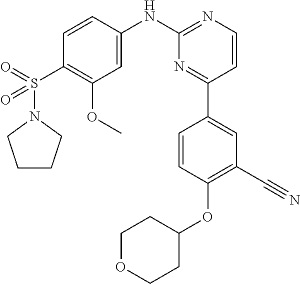 [SMILES: COc1cc(Nc2nccc(-c3ccc(OC4CCOCC4)c(C#N)c3)n2)ccc1S(=O)(=O)N1CCCC1] | 5-(2-{[3-methoxy-4- (pyrrolidin-1- ylsulfonyl)phenyl] amino}pyrimidin- 4-yl)- 2-(tetrahydro-2H- pyran-4- yloxy)benzonitrile | ¹H NMR (MeOH d-4) δ 8.54 (d, 1H), 8.46 d, 1H), 8.31 (dd, 1H), 7.99 (br s, 1H), 7.81 (d, 1H), 7.29- 7.22 (s, 2H), 4.89-4.85 (m, 1H), 4.59 (s, 3H), 4.08-4.03 (m, 5H), 3.74-3.69 (m, 2H), 3.40-3.35 (m, 6H), 2.16-2.11 (m, 2H), 1.97-1.85 (t, 7H). LC-MS [M + H]⁺ 536.1913 |
| 395 | 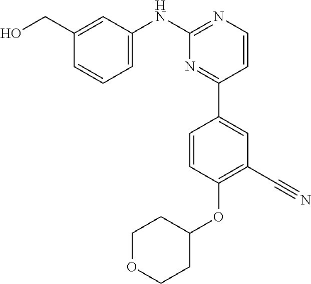 [SMILES: N#Cc1cc(-c2ccnc(Nc3cccc(CO)c3)n2)ccc1OC1CCOCC1] | 5-(2-{[3- (hydroxymethyl) phenyl] amino}pyrimidin- 4-yl)-2-(tetrahydro- 2H-pyran-4- yloxy)benzonitrile | ¹H NMR (CDCl₃) δ 8.46 (d, 1H), 8.37 (s, 1H), 8.23 (d, 1H), 7.88 (s, 1H), 7.49 (d, 1H), 7.29-7.32 (m, 2H), 7.08-7.04 (m, 3H), 4.76-4.72 (m, 3H), 4.06-4.01 (m, 2H), 3.69- 3.63 (m, 2H), 2.21 (t, 3H), 2.12- 2.05 (m, 2H), 1.96-1.88 (m, 2H). LC-MS [M + H]⁺ 403.1703. |
| 396 | 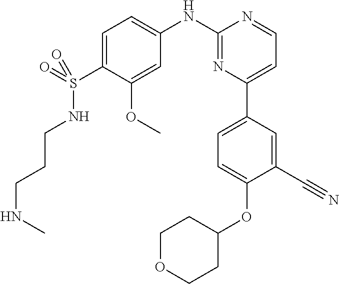 [SMILES: CNCCCNS(=O)(=O)c1ccc(Nc2nccc(-c3ccc(OC4CCOCC4)c(C#N)c3)n2)cc1OC] | 4-({4-[3-cyano-4- (tetrahydro-2H- pyran-4- yloxy)phenyl] pyrimidin-2-yl} amino)-2- methoxy-N-[3- (methylamino)propyl] benzenesulfonamide] | ¹H NMR (CDCl₃) δ 8.53 (d, 1H), 8.41 (s, 1H), 8.28 (dd, 1H), 7.93 (s, 1H), 7.70 (d, 1H), 7.25 (m, 3H), 4.85-4.81 (m, 1H), 4.10-4.02 (m, 5H), 3.73-3.67 (m, 2H), 3.13 (t, 3H), 2.97 (t, 2H), 2.70 (s, 3H), 2.15-2.10 (m, 2H), 1.97-1.88 (m, 4H). LC-MS [M + H]⁺ 553.2148. |
| 397 | 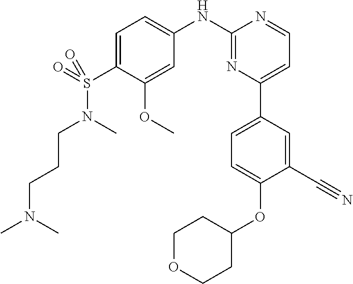 [SMILES: COc1cc(Nc2nccc(-c3ccc(OC4CCOCC4)c(C#N)c3)n2)ccc1S(=O)(=O)N(C)CCCN(C)C] | 4-({4-[3-cyano-4- (tetrahydro-2H- pyran-4- yloxy)phenyl] pyrimidin-2-yl} amino)-N-[3- (dimethylamino) propyl]-2-methoxy-N- methylbenzene- sulfonamide] | ¹H NMR (CDCl₃) δ 8.54 (d, 1H), 8.38 (d, 2H), 8.22 (dd, 1H), 7.89- 7.84 (m, 2H), 7.62 (br s, 1H), 7.18 (d, 1H), 7.10 (d, 1H), 7.04 (dd, 1H), 4.78 (m, 1H), 4.07-4.00 (m, 5H), 3.70-3.65 (m, 2H), 3.20 (t, 3H), 2.86 (s, 3H). LC-MS [M + H]⁺ 581.2592 |
| 398 | 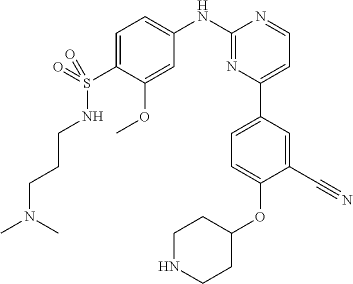 [SMILES: COc1cc(Nc2nccc(-c3ccc(OC4CCNCC4)c(C#N)c3)n2)ccc1S(=O)(=O)NCCCN(C)C] | 4-({4-[3-cyano-4- (piperidin-4- yloxy)phenyl] pyrimidin-2-yl} amino)-N-[3- (dimethylamino) propyl]-2- methoxybenzene- sulfonamide] | ¹H NMR (MeOH d-4) δ 8.54 (d, 1H), 8.45 (d, 1H), 8.31 (dd, 1H), 7.97 (br s, 1H), 7.79 (d, 1H), 7.43 (s, 2H), 4.86 (m, 1H), 4.31 (s, 12H), 4.01 (s, 3H), 3.66 (t, 2H), 3.32-3.25 (m, 2H), 3.08-3.00 (m, 2H), 2.98 (t, 2H), 2.24-2.16 (m, 2H), 2.05-1.98 (m, 2H), 1.71 (m, 2H). LC-MS [M + H]⁺ 566.2581 |
| 399 | 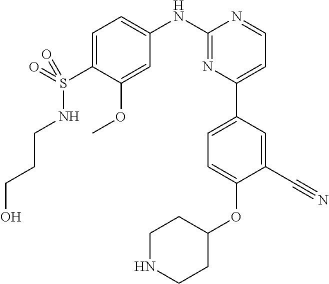 [SMILES: COc1cc(Nc2nccc(-c3ccc(OC4CCNCC4)c(C#N)c3)n2)ccc1S(=O)(=O)NCCCO] | 4-({4-[3-cyano-4- (piperidin-4- yloxy)phenyl] pyrimidin- 2-yl}amino)-N- (3-hydroxypropyl)-2- methoxybenzene- sulfonamide | ¹H NMR (MeOH d-4) δ 8.54 (s, 1H), 8.45 (s, 1H), 8.29 (d, 1H), 7.99 (br s, 1H), 7.78 (d, 1H), 7.47 (s, 2H), 7.28-7.20 (m, 3H), 3.37- 3.32 (m, 2H), 3.26-3.18 (m, 5H), 2.42-2.34 (m, 2H), 2.24 (s, 3H), 2.18-2.08 (m, 2H), 1.96-1.84 (m, 2H), 1.74-1.64 (m, 2H). LC-MS [M + H]⁺ 539.2112 |
| 400 | 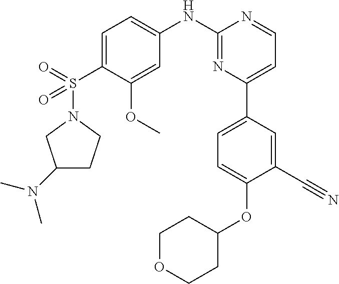 [SMILES: COc1cc(Nc2nccc(-c3ccc(OC4CCOCC4)c(C#N)c3)n2)ccc1S(=O)(=O)N1CCC(N(C)C)C1] | 5-{2-[(4-{[3- (dimethylamino) pyrrolidin-1- yl]sulfonyl}-3- methoxyphenyl) amino]pyrimidin- 4-yl}-2- (tetrahydro-2H- pyran-4- yloxy)benzonitrile | ¹H NMR (DMSO d-6) δ 10.20 (s, 1H), 8.65 (d, 1H), 8.60 (d, 1H), 8.47 (dd, 1H), 7.95 (s, 1H), 7.68- 7.55 (m, 3H), 7.43 (dd, 1H), 4.98- 4.90 (m, 1H), 4.45-4.38 (m, 1H), 3.93 (s, 3H), 3.91-3.86 (m, 2H), 3.60-3.53 (m, 2H), 2.75 (s, 3H), 2.64-2.57 (m, 1H), 2.48-2.42 (m, 1H), 2.20 (t, 1H), 2.14 (s, 3H), 2.08-2.01 (m, 3H), 1.87-1.79 (m, 1H), 1.72-1.61 (m, 3H). LC-MS [M + H]⁺ 579.2322 |
| 401 | 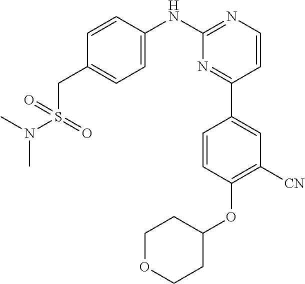 [SMILES: CN(C)S(=O)(=O)Cc1ccc(Nc2nccc(-c3ccc(OC4CCOCC4)c(C#N)c3)n2)cc1] | 1-[4-({4-[3-Cyano-4- (tetrahydro-2H- pyran-4- yloxy)phenyl] pyrimidin-2- yl}amino)phenyl]- N,N- dimethylmethane- sulfonamide | ¹H NMR (DMSO-d₆) δ 9.82 (s, 1H), 8.58-8.55 (m, 2H), 8.48-8.45 (m, 1H), 7.84-7.81 (m, 2H), 7.58-7.50 (m, 2H), 7.36-7.34 (m, 2H), 4.96- 4.94 (m, 1H), 4.36 (s, 2H), 3.90- 3.85 (m, 2H), 3.58-3.53 (m, 2H), 2.72 (s, 6H), 2.08-2.03 (m, 2H), 1.71-1.67 (m, 2H). LC-MS [M + H]⁺ 494.1856 |
| 402 | 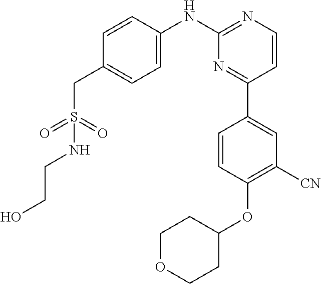 [SMILES: N#Cc1cc(-c2ccnc(Nc3ccc(CS(=O)(=O)NCCO)cc3)n2)ccc1OC1CCOCC1] | 1-[4-({4-[3-Cyano-4- (tetrahydro-2H- pyran-4- yloxy)phenyl] pyrimidin-2- yl}amino)phenyl]-N- (2- hydroxyethyl) methanesulfonamide | ¹H NMR (CDCl₃) δ 8.46 (d, 1H), 8.31-8.26 (m, 2H), 7.73-7.71 (m, 2H), 7.43-7.41 (m, 2H), 7.14-7.11 (m, 2H), 4.81-4.77 (m, 1H), 4.29 (s, 2H), 4.08-4.02 (m, 2H), 3.71-3.62 (m, 4H), 3.13-3.11 (m, 2H), 2.15- 2.06 (m, 2H), 1.98-1.91 (m, 2H). LC-MS [M + H]⁺ 510.1808 |
| 403 | 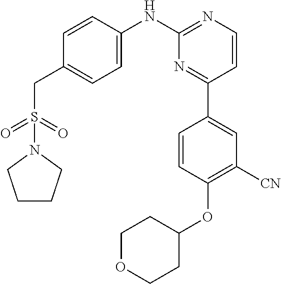 [SMILES: N#Cc1cc(-c2ccnc(Nc3ccc(CS(=O)(=O)N4CCCC4)cc3)n2)ccc1OC1CCOCC1] | 5-[2-({4- [(Pyrrolidin-1- ylsulfonyl)methyl] phenyl}amino) pyrimidin-4-yl]-2- (tetrahydro-2H- pyran-4- yloxy)benzonitrile | ¹H NMR (CDCl₃) δ 8.49 (d, 1H), 8.30-8.25 (m, 2H), 7.73-7.70 (m, 2H), 7.41-7.39 (m, 2H), 7.27-7.22 (m, 1H), 7.12-7.10 (m, 2H), 4.79- 4.74 (m, 1H), 4.25 (s, 2H), 4.07- 4.02 (m, 2H), 3.70-3.64 (m, 2H), 3.23-3.16 (m, 4H), 2.14-2.07 (m, 2H), 1.98-1.78 (m, 5H). LC-MS [M + H]⁺ 520.2007 |
| 404 | 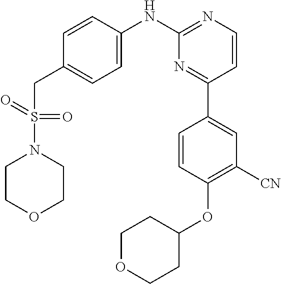 [SMILES: N#Cc1cc(-c2ccnc(Nc3ccc(CS(=O)(=O)N4CCOCC4)cc3)n2)ccc1OC1CCOCC1] | 5-[2-({4- [(Morpholin-4- ylsulfonyl)methyl] phenyl}amino) pyrimidin-4-yl]-2- (tetrahydro-2H- pyran-4- yloxy)benzonitrile | ¹H NMR (CDCl₃) δ 8.51 (d, 1H), 8.30-8.25 (m, 2H), 7.76-7.72 (m, 2H), 7.43-7.38 (m, 3H), 7.13-7.11 (m, 2H), 4.79-4.74 (m, 1H), 4.24 (s, 2H), 4.07-4.02 (m, 2H), 3.70-3.64 (m, 6H), 3.17-3.15 (m, 4H), 2.14- 2.07 (m, 2H), 1.98-1.89 (m, 2H). LC-MS [M + H]⁺ 536.1926 |
| 405 | 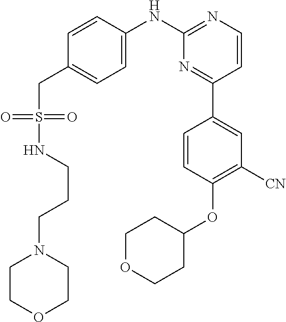 [SMILES: N#Cc1cc(-c2ccnc(Nc3ccc(CS(=O)(=O)NCCCN4CCOCC4)cc3)n2)ccc1OC1CCOCC1] | 1-[4-({4-[3-Cyano-4- (tetrahydro-2H- pyran-4- yloxy)phenyl] pyrimidin-2- yl}amino)phenyl]-N- [3-(morpholin-4- yl)propyl] methanesulfonamide | ¹H NMR (CDCl₃) δ 8.47 (d, 1H), 8.29 (d, 1H), 8.25-8.22 (m, 1H), 7.73-7.70 (m, 2H), 7.43-7.41 (m, 3H), 7.12-7.09 (m, 2H), 4.79-4.74 (m, 1H), 4.24 (s, 2H), 4.07-4.02 (m, 2H), 3.70-3.64 (m, 2H), 3.47 (bs, 4H), 3.21-3.18 (m, 2H), 2.46-2.43 (m, 2H), 2.32 (bs, 4H), 2.14-2.05 (m, 2H), 1.97-1.89 (m, 2H), 1.72- 1.67 (m, 2H), LC-MS [M + H]⁺ 593.2497 |
| 406 | 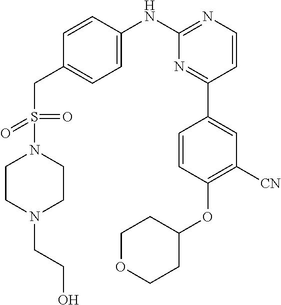 [SMILES: N#Cc1cc(-c2ccnc(Nc3ccc(CS(=O)(=O)N4CCN(CCO)CC4)cc3)n2)ccc1OC1CCOCC1] | 5-(2-{[4-({[4-(2- Hydroxyethyl) piperazin-1- yl]sulfonyl}methyl) phenyl]amino} pyrimidin-4-yl)-2- (tetrahydro-2H- pyran-4- yloxy)benzonitrile | ¹H NMR (CDCl₃) δ 8.50 (d, 1H), 8.30-8.25 (m, 2H), 7.74 (d, 2H), 7.41-7.38 (m, 3H), 7.13-7.11 (m, 2H), 4.79-4.74 (m, 1H), 4.22 (s, 2H), 4.07-4.03 (m, 2H), 3.70-3.64 (m, 2H), 3.61-3.58 (m, 2H), 3.21- 3.19 (m, 4H), 2.56-2.49 (m, 6H), 2.14-2.07 (m, 2H), 1.97-1.89 (m, 2H). LC-MS [M + H]⁺ 579.2342 |
| 407 | 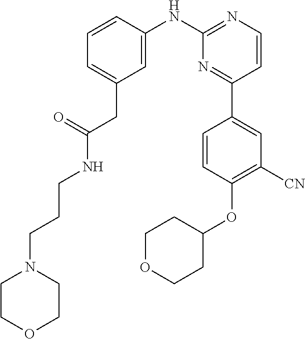 [SMILES: N#Cc1cc(-c2ccnc(Nc3cccc(CC(=O)NCCCN4CCOCC4)c3)n2)ccc1OC1CCOCC1] | 2-[3-({4-[3-Cyano-4- (tetrahydro-2H- pyran-4- yloxy)phenyl] pyrimidin-2- yl}amino)phenyl]-N- [3-(morpholin-4- yl)propyl]acetamide | ¹H NMR (CDCl₃) δ 8.48 (d, 1H), 8.30-8.26 (m, 2H), 7.71 (s, 1H), 7.57 (d, 1H), 7.39-7.34 (m, 2H), 7.13-7.08 (m, 2H), 6.96 (d, 1H), 6.44 (s, 1H), 4.78-4.74 (m, 1H), 4.06-4.02 (m, 2H), 3.71-3.62 (m, 2H), 3.60-3.56 (m, 6H), 3.46-3.30 (m, 2H), 2.34-2.31 (m, 6H), 2.13- 2.07 (m, 2H), 1.94-1.91 (m, 2H), 1.65-1.59 (m, 2H). LC-MS [M + H]⁺ 557.2880 |
| 408 | 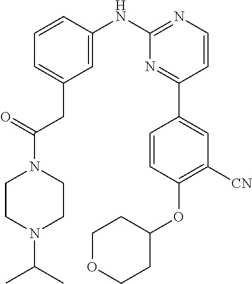 [SMILES: CC(C)N1CCN(C(=O)Cc2cccc(Nc3nccc(-c4ccc(OC5CCOCC5)c(C#N)c4)n3)c2)CC1] | 5-{2-[(3-{2-Oxo-2- [4-(propan-2- yl)piperazin-1- yl]ethyl}phenyl) amino]pyrimidin- 4-yl}-2- (tetrahydro-2H- pyran-4- yloxy)benzonitrile | ¹H NMR (CDCl₃) δ 8.47 (d, 1H), 8.35-8.30 (m, 2H), 7.70 (s, 1H), 7.53 (d, 1H), 7.37-7.29 (m, 2H), 7.14 (d, 1H), 7.08 (d, 1H), 6.93 (d, 1H), 4.79-4.75 (m, 1H), 4.06-4.02 (m, 2H), 3.78 (s, 2H), 3.67-3.63 (m, 4H), 3.48-3.46 (m, 2H), 2.65-2.62 (m, 1H), 2.44-2.42 (m, 2H), 2.36- 2.33 (m, 2H), 2.11-2.07 (m, 2H), 1.96-1.89 (m, 2H), 0.97 (d, 6H). LC-MS [M + H]⁺ 541.2930 |
| 409 | 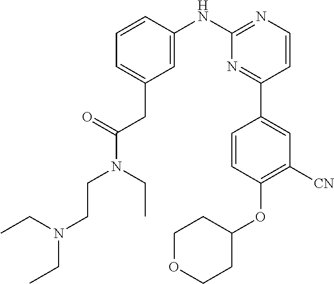 [SMILES: CCN(CC)CCN(CC)C(=O)Cc1cccc(Nc2nccc(-c3ccc(OC4CCOCC4)c(C#N)c3)n2)c1] | 2-[3-({4-[3-Cyano-4- (tetrahydro-2H- pyran-4- yloxy)phenyl] pyrimidin-2- yl}amino)phenyl]-N- [2- (diethylamino)ethyl]- N-ethylacetamide | ¹H NMR (CDCl₃) δ 8.47 (d, 1H), 8.35-8.30 (m, 2H), 7.63-7.58 (m, 2H), 7.34-7.24 (m, 2H), 7.14-7.08 (m, 2H), 6.95 (d, 1H), 4.79-4.75 (m, 2H), 4.06-4.04 (m, 2H), 3.76 (d, 2H), 3.69-3.64 (m, 2H), 3.49- 3.31 (m, 4H), 2.58-2.48 (m, 5H), 2.11-2.03 (m, 2H), 1.97-1.91 (m, 2H), 1.15-1.11 (m, 3H), 1.02-0.98 (m, 5H). LC-MS [M + H]⁺ 557.3246 |
| 410 | 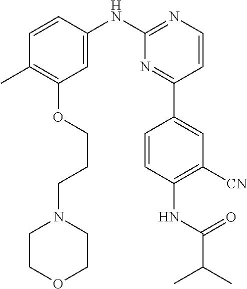 [SMILES: Cc1ccc(Nc2nccc(-c3ccc(NC(=O)C(C)C)c(C#N)c3)n2)cc1OCCCN1CCOCC1] | N-{2-Cyano-4-[2- ({4-methyl-3-[3- (morpholin-4- ylpropoxy]phenyl} amino)pyrimidin-4- yl]phenyl}-2- methylpropanamide | ¹H NMR (CDCl₃) δ 8.61 (d, 1H), 8.47 (d, 1H), 8.37 (s, 1H), 8.24 (d, 1H), 7.81 (s, 1H), 7.44 (d, 1H), 7.32 (s, 1H), 7.10 (d, 2H), 6.97- 6.94 (m, 1H), 4.10-4.08 (m, 2H), 3.73-3.70 (m, 4H), 2.70-2.63 (m, 1H), 2.59-2.49 (m, 6H), 2.20-2.16 (m, 5H), 2.08-2.02 (m, 2H), 1.33 (d, 6H). LC-MS [M + H]⁺ 515.2777 |
| 411 | 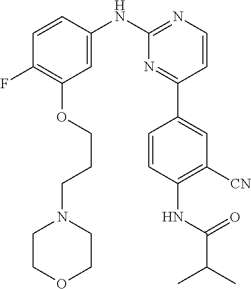 [SMILES: CC(C)C(=O)Nc1ccc(-c2ccnc(Nc3ccc(F)c(OCCCN4CCOCC4)c3)n2)cc1C#N] | N-{2-Cyano-4-[2- ({4-fluoro-3-[3- (morpholin-4- yl)propoxy]phenyl} amino)pyrimidin-4- yl]phenyl}-2- methylpropanamide | ¹H NMR (CDCl₃) δ 8.64 (d, 1H), 8.49 (d, 1H), 8.36 (s, 1H), 8.25 (d, 1H), 7.80 (s, 1H), 7.59 (d, 1H), 7.17 (s, 1H), 7.12 (d, 1H), 7.08- 6.98 (m, 2H), 4.18-4.15 (m, 2H), 3.71-3.69 (m, 4H), 2.70-2.63 (m, 1H), 2.59-2.47 (m, 6H), 2.08-2.02 (m, 2H), 1.33 (d, 6H). LC-MS [M + H]⁺ 519.2537 |
| 412 | 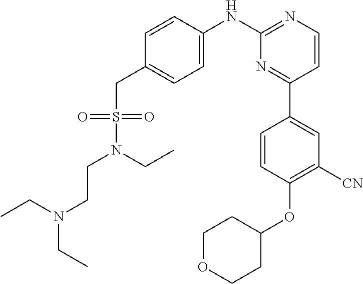 [SMILES: CCN(CC)CCN(CC)S(=O)(=O)Cc1ccc(Nc2nccc(-c3ccc(OC4CCOCC4)c(C#N)c3)n2)cc1] | 1-[4-({4-[3-Cyano-4- (tetrahydro-2H- pyran-4- yloxy)phenyl] pyrimidin-2- yl}amino)phenyl]-N- [2- (diethylamino)ethyl]- N- ethylmethane- sulfonamide | ¹H NMR (CDCl₃) δ 8.49 (d, 1H), 8.28 (d, 2H), 7.71 (d, 2H), 7.40 (d, 2H), 7.32 (s, 1H), 7.13-7.11 (m, 2H), 4.77-4.75 (m, 1H), 4.27 (s, 2H), 4.07-4.03 (m, 2H), 3.69-3.65 (m, 2H), 3.20-3.11 (m, 4H), 2.58- 2.22 (m, 5H), 2.12-2.05 (m, 2H), 1.96-1.90 (m, 2H), 1.15-1.11 (m, 3H), 1.03-0.99 (m, 5H). LC-MS [M + H]⁺ 593.2909 |
| 413 | 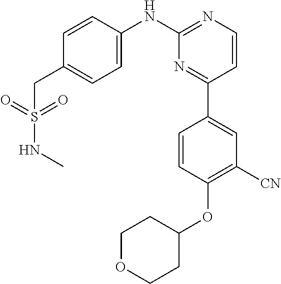 [SMILES: CNS(=O)(=O)Cc1ccc(Nc2nccc(-c3ccc(OC4CCOCC4)c(C#N)c3)n2)cc1] | 1-[4-({4-[3-cyano-4- (tetrahydro-2H- pyran-4- yloxy)phenyl] pyrimidin-2- yl}amino)phenyl]-N- methylmethane- sulfonamide | ¹H NMR (DMSO-d₆) δ 9.80 (s, 1H), 8.56 (d, 2H), 8.46 (d, 1H), 7.81 (d, 2H), 7.57 (d, 1H), 7.50 (d, 1H), 7.30 (d, 2H), 6.91-6.88 (m, 1H), 4.97-4.93 (m, 1H), 4.27 (s, 2H), 3.89-3.85 (m, 2H), 3.58-3.54 (m, 2H), 2.57 (d, 3H), 2.07-2.03 (m, 2H), 1.72-1.65 (m, 2H). LC-MS [M + H]⁺ 480.1704 |
| 414 | 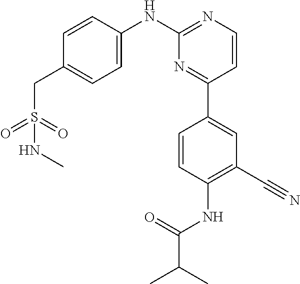 [SMILES: CNS(=O)(=O)Cc1ccc(Nc2nccc(-c3ccc(NC(=O)C(C)C)c(C#N)c3)n2)cc1] | N-{2-cyano-4-[2- ({4- [(methylsulfamoyl) methyl]phenyl}amino) pyrimidin-4- yl]phenyl}-2- methylpropanamide | ¹H NMR (CDCl₃) δ 8.53 (d, 1H), 8.48 (d, 1H), 8.33 (d, 1H), 8.27- 8.24 (m, 1H), 7.72 (d, 2H), 7.40 (d, 2H), 7.15 (d, 1H), 4.26 (s, 2H), 2.75-2.67 (m, 4H), 1.32 (d, 6H). LC-MS [M + H]⁺ 465.1714 |
| 415 | 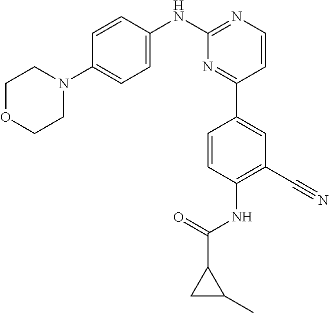 [SMILES: CC1CC1C(=O)Nc1ccc(-c2ccnc(Nc3ccc(N4CCOCC4)cc3)n2)cc1C#N] | N-[2-cyano-4-(2-{[4- (morpholin-4- yl)phenyl]amino} pyrimidin-4-yl)phenyl]- 2- methylcyclopropane- carboxamide | ¹H NMR (CDCl₃) δ 8.59 (d, 1H), 8.44 (d, 1H), 8.32 (d, 1H), 8.22 (d, 1H), 8.19 (d, 1H), 7.94 (s, 1H), 7.54 (d, 2H), 7.26 (s, 1H), 7.04 (d, 1H), 6.95 (d, 2H), 3.90-3.87 (m, 4H), 3.16-3.14 (m, 4H), 1.59-1.52 (m, 1H), 1.39-1.32 (m, 2H), 1.20 (d, 3H), 0.85-0.81 (m, 1H). LC-MS [M + H]⁺ 455.2180 |
| 416 | 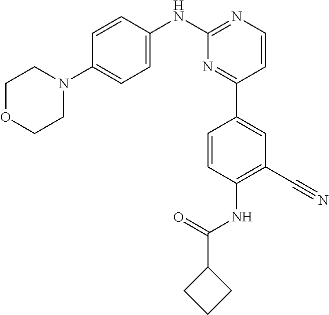 [SMILES: N#Cc1cc(-c2ccnc(Nc3ccc(N4CCOCC4)cc3)n2)ccc1NC(=O)C1CCC1] | N-[2-cyano-4-(2-{[4- (morpholin-4- yl)phenyl]amino} pyrimidin-4- yl)phenyl] cyclobutane- carboxamide | ¹H NMR (CDCl₃) δ 8.64 (d, 1H), 8.45 (d, 1H), 8.33 (s, 1H), 8.24 (d, 1H), 7.64 (s, 1H), 7.54 (d, 2H), 7.18 (s, 1H), 7.05 (d, 1H), 6.96 (d, 2H), 3.90-3.88 (m, 4H), 3.34-3.25 (m, 1H), 3.17-3.14 (m, 4H), 2.49- 2.39 (m, 2H), 2.36-2.29 (m, 2H), 2.13-2.06 (m, 1H), 2.04-1.93 (m, 1H). LC-MS [M + H]⁺ 455.2184 |
| 417 | 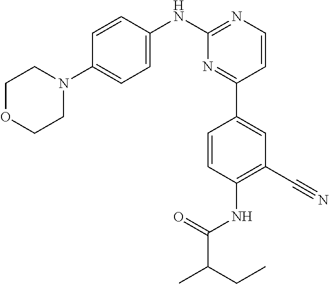 [SMILES: CCC(C)C(=O)Nc1ccc(-c2ccnc(Nc3ccc(N4CCOCC4)cc3)n2)cc1C#N] | N-[2-cyano-4-(2-{[4- (morpholin-4- yl)phenyl]amino} pyrimidin-4-yl)phenyl]- 2-methylbutanamide | ¹H NMR (CDCl₃) δ 8.63 (d, 1H), 8.45 (d, 1H), 8.33 (s, 1H), 8.24 (d, 1H), 7.77 (s, 1H), 7.54 (d, 2H), 7.27 (d, 1H), 7.05 (d, 2H), 6.96 (d, 2H), 3.88-3.85 (m, 4H), 3.20-3.14 (m, 4H), 2.47-2.39 (m, 1H), 1.88- 1.77 (m, 1H), 1.66-1.55 (m, 1H), 1.30 (d, 3H), 1.03-0.99 (d, 3H). LC-MS [M + H]⁺ 457.2340 |
| 418 | 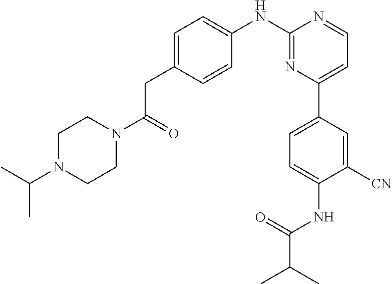 [SMILES: CC(C)C(=O)Nc1ccc(-c2ccnc(Nc3ccc(CC(=O)N4CCN(C(C)C)CC4)cc3)n2)cc1C#N] | N-(2-cyano-4-{2-[(4- {2-oxo-2-[4-(propan- 2-yl)piperazin-1- yl]ethyl}phenyl) amino]pyrimidin-4- yl}phenyl)-2- methylpropanamide | ¹H NMR (CDCl₃) δ 8.59 (d, 1H), 8.48 (d, 1H), 8.32 (d, 1H), 8.25- 8.22 (m, 1H), 7.89 (s, 1H), 7.63- 7.60 (m, 2H), 7.39 (s, 1H), 7.27- 7.23 (m, 2H), 7.09 (d, 1H), 3.73 (s, 2H), 3.68-3.66 (m, 2H), 3.50-3.48 (m, 2H), 2.71-2.64 (m, 2H), 2.49- 2.47 (m, 2H), 2.40-2.37 (m, 2H), 1.33 (d, 6H), 1.01 (d, 6H). LC-MS [M + H]⁺ 526.2935 |
| 419 | 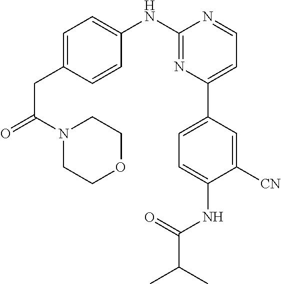 [SMILES: CC(C)C(=O)Nc1ccc(-c2ccnc(Nc3ccc(CC(=O)N4CCOCC4)cc3)n2)cc1C#N] | N-{2-cyano-4-[2- ({4-[2-(morpholin-4- yl)-2- oxoethyl]phenyl} amino)pyrimidin-4- yl]phenyl}-2- methylpropanamide | ¹H NMR (CDCl₃) δ 8.47 (d, 1H), 8.43-8.40 (m, 1H), 8.34 (d, 1H), 8.27-8.24 (m, 1H), 7.68-7.66 (m, 2H), 7.24 (d, 2H), 7.13 (d, 1H), 3.74 (s, 2H), 3.69-3.65 (m, 4H), 3.56-3.49 (m, 4H), 2.82-2.69 (m, 1H), 1.32 (d, 6H). LC-MS [M + H]⁺ 485.2297 |
| 420 | 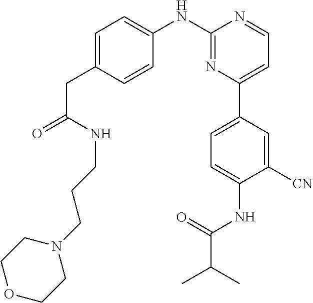 [SMILES: CC(C)C(=O)Nc1ccc(-c2ccnc(Nc3ccc(CC(=O)NCCCN4CCOCC4)cc3)n2)cc1C#N] | N-[2-cyano-4-(2-{[4- (2-{[3-(morpholin-4- yl)propyl]amino}-2- oxoethyl)phenyl] amino}pyrimidin-4- yl)phenyl]-2- methylpropanamide | ¹H NMR (CDCl₃ + MeOH-d₄) δ 8.52-8.47 (m, 2H), 8.35 (d, 1H), 8.27-8.25 (m, 1H), 7.69-7.66 (m, 2H), 7.31-7.26 (m, 2H), 7.13 (d, 1H), 3.64-3.62 (m, 4H), 3.55 (s, 2H), 3.30-3.27 (m, 2H), 2.77-2.67 (m, 3H), 2.39-2.32 (m, 6H), 1.68- 1.62 (m, 2H), 1.32 (s, 6H). LC-MS [M + H]⁺ 542.2892 |
| 421 | 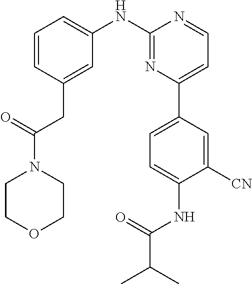 [SMILES: CC(C)C(=O)Nc1ccc(-c2ccnc(Nc3cccc(CC(=O)N4CCOCC4)c3)n2)cc1C#N] | N-{2-cyano-4-[2- ({3-[2-(morpholin-4- yl)-2- oxoethyl]phenyl} amino)pyrimidin-4- yl]phenyl}-2- methylpropanamide | ¹H NMR (CDCl₃) δ 8.61 (d, 1H), 8.49 (d, 1H), 8.l36 (d, 1H), 8.27- 8.24 (m, 1H), 7.87 (s, 1H), 7.69- 7.68 (m, 1H), 7.51-7.49 (m, 1H), 7.40 (s, 1H), 7.33-7.29 (m, 1H), 7.11 (d, 1H), 6.94-6.92 (m, 1H), 3.78 (s, 2H), 3.68-3.64 (m, 4H), 3.55-3.48 (m, 4H), 2.71-2.64 (m, 1H), 1.33 (d, 6H). LC-MS [M + H]⁺ 485.2290 |
| 422 | 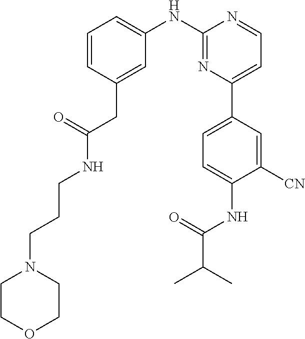 [SMILES: CC(C)C(=O)Nc1ccc(-c2ccnc(Nc3cccc(CC(=O)NCCCN4CCOCC4)c3)n2)cc1C#N] | N-[2-cyano-4-(2-{[3- (2-{[3-(morpholin-4- yl)propyl]amino}-2- oxoethyl)phenyl] amino}pyrimidin-4- yl)phenyl]-2- methylpropanamide | ¹H NMR (CDCl₃ + MeOH-d₄) δ 8.51-8.47 (m, 2H), 8.36 (d, 1H), 8.28-8.25 (m, 1H), 7.67-7.61 (m, 2H), 7.37-7.31 (m, 1H), 7.14 (d, 1H), 6.97 (d, 1H), 3.63-3.61 (m, 4H), 3.58 (s, 2H), 3.30-3.27 (m, 2H), 2.74-2.69 (m, 3H), 2.37-2.31 (m, 6H), 1.68-1.61 (m, 2H), 1.32 (d, 6H). LC-MS [M + H]⁺ 542.2870 |
| 423 | 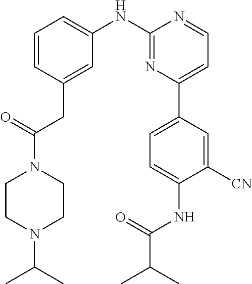 [SMILES: CC(C)C(=O)Nc1ccc(-c2ccnc(Nc3cccc(CC(=O)N4CCN(C(C)C)CC4)c3)n2)cc1C#N] | N-(2-cyano-4-{2-[(3- {2-oxo-2-[4-(propan- 2-yl)piperazin-1- yl]ethyl}phenyl) amino]pyrimidin-4- yl}phenyl)-2- methylpropanamide | ¹H NMR (CDCl₃) δ 8.56 (d, 1H), 8.48 (d, 1H), 8.34 (s, 1H), 8.24- 8.22 (m, 1H), 7.98 (s, 1H), 7.61 (d, 2H), 7.51 (d, 1H), 7.30-7.26 (m, 1H), 7.09 (d, 1H), 6.93 (d, 1H), 3.78 (s, 2H), 3.68-3.66 (m, 2H), 3.51-3.48 (m, 2H), 2.72-2.62 (m, 2H), 2.48-2.45 (m, 2H), 2.39-2.36 (m, 2H), 1.32 (d, 6H), 0.99 (d, 6H). LC-MS [M + H]⁺ 526.2932 |
| 424 | 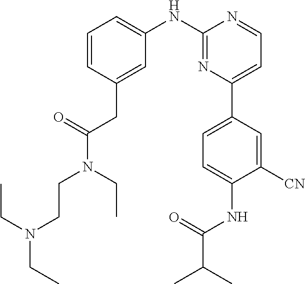 [SMILES: CCN(CC)CCN(CC)C(=O)Cc1cccc(Nc2nccc(-c3ccc(NC(=O)C(C)C)c(C#N)c3)n2)c1] | N-[2-cyano-4-(2-{[3- (2-{[2- (diethylamino)ethyl] (ethyl)amino}-2- oxoethyl)phenyl] amino}pyrimidin-4- yl)phenyl]-2- methylpropanamide | ¹H NMR (CDCl₃) δ 8.56 (d, 1H), 8.48 (d, 1H), 8.34 (s, 1H), 8.26- 8.24 (m, 1H), 8.03-7.97 (m, 1H), 7.65-7.50 (m, 3H), 7.31-7.27 (m, 1H), 7.09 (d, 1H), 6.94 (d, 1H), 3.76 (d, 2H), 3.75-3.50 (m, 4H), 2.79-2.65 (m, 5H), 2.56-2.51 (m, 2H), 1.32 (d, 6H), 1.19-1.11 (m, 6H), 1.03-0.99 (m, 3H). LC-MS [M + H]⁺ 542.3235 |
| 425 | 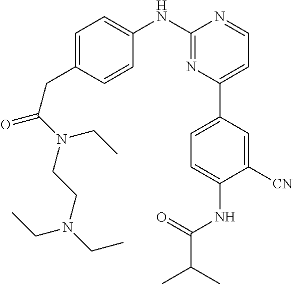 [SMILES: CCN(CC)CCN(CC)C(=O)Cc1ccc(Nc2nccc(-c3ccc(NC(=O)C(C)C)c(C#N)c3)n2)cc1] | N-[2-cyano-4-(2-{[4- (2-{[2- (diethylamino)ethyl] (ethyl)amino}-2- oxoethyl)phenyl] amino}pyrimidin-4- yl)phenyl]-2- methylpropanamide | ¹H NMR (CDCl₃) δ 8.60 (d, 1H), 8.48 (d, 1H), 8.33 (s, 1H), 8.26- 8.24 (m, 1H), 7.88 (s, 1H), 7.60 (d, 2H), 7.30 (s, 1H), 7.26-7.24 (m, 2H), 7.09 (d, 1H), 3.72 (d, 2H), 3.45-3.19 (m, 4H), 2.71-2.50 (m, 7H), 1.33 (d, 6H), 1.17-1.12 (m, 3H), 1.05-1.01 (m, 6H). LC-MS [M + H]⁺ 542.3251 |
| 426 | 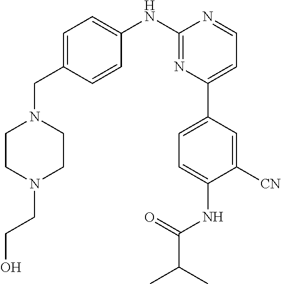 [SMILES: CC(C)C(=O)Nc1ccc(-c2ccnc(Nc3ccc(CN4CCN(CCO)CC4)cc3)n2)cc1C#N] | N-(2-cyano-4-{2-[(4- {[4-(2- hydroxyethyl) piperazin-1- yl]methyl}phenyl) amino]pyrimidin-4- yl}phenyl)-2- methylpropanamide | ¹H NMR (CDCl₃) δ 8.62 (d, 1H), 8.48 (d, 1H), 8.34 (s, 1H), 8.27- 8.25 (m, 1H), 7.86 (s, 1H), 7.62 (d, 2H), 7.47 (s, 1H), 7.32 (d, 2H), 7.10 (d, 1H), 3.66-3.62 (m, 3H), 3.53 (s, 2H), 2.71-2.47 (m, 11H), 1.33 (d, 6H). LC-MS [M + H]⁺ 500.2761 |
| 427 | 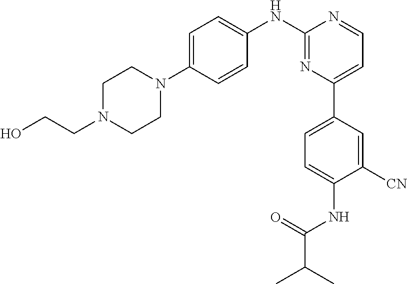 [SMILES: CC(C)C(=O)Nc1ccc(-c2ccnc(Nc3ccc(N4CCN(CCO)CC4)cc3)n2)cc1C#N] | N-{2-cyano-4-[2- ({4-[4-(2- hydroxyethyl) piperazin-1- yl]phenyl}amino) pyrimidin-4-yl] phenyl}-2- methylpropanamide | ¹H NMR (CDCl₃) δ 8.60 (d, 1H), 8.44 (d, 1H), 8.33 (s, 1H), 8.25- 8.23 (m, 1H), 7.82 (s, 1H), 7.52 (d, 2H), 7.26-7.24 (m, 1H), 7.04 (d, 1H), 6.96 (d, 2H), 3.69-3.66 (m, 2H), 3.20 (bs, 4H), 2.72-2.58 (m, 8H), 1.33 (d, 6H). LC-MS [M + H]⁺ 486.2591 |
| 428 | 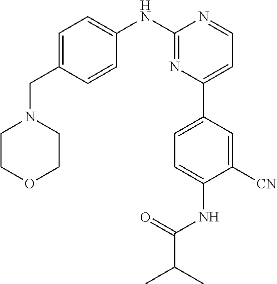 [SMILES: CC(C)C(=O)Nc1ccc(-c2ccnc(Nc3ccc(CN4CCOCC4)cc3)n2)cc1C#N] | N-[2-cyano-4-(2-{[4- (morpholin-4- ylmethyl)phenyl] amino}pyrimidin-4- yl)phenyl]-2- methylpropanamide | ¹H NMR (CDCl₃) δ 8.64 (d, 1H), 8.50 (d, 1H), 8.35 (s, 1H), 8.27- 8.17 (m, 1H), 7.82 (s, 1H), 7.62 (d, 2H), 7.40-7.32 (m, 2H), 7.22 (d, 1H), 7.11 (d, 1H), 3.73-3.71 (m, 4H), 3.49 (s, 2H), 2.70-2.62 (m, 1H), 2.47 (bs, 4H), 1.33 (d, 6H). LC-MS [M + H]⁺ 457.2338 |
| 429 | 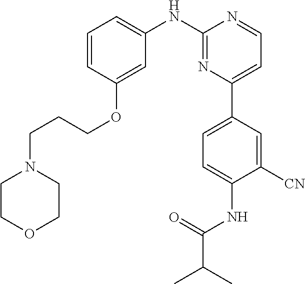 [SMILES: CC(C)C(=O)Nc1ccc(-c2ccnc(Nc3cccc(OCCCN4CCOCC4)c3)n2)cc1C#N] | N-{2-cyano-4-[2- ({3-[3-(morpholin-4- yl)propoxy]phenyl} amino)pyrimidin-4- yl]phenyl}-2- methylpropanamide | ¹H NMR (CDCl₃) δ 8.63 (d, 1H), 8.50 (d, 1H), 8.38 (d, 1H), 8.27- 8.24 (m, 1H), 7.80 (s, 1H), 7.49- 7.48 (m, 1H), 7.30-7.23 (m, 2H), 7.12-7.09 (m, 2H), 6.65-6.62 (m, 1H), 4.09-4.06 (m, 2H), 3.73-3.71 (m, 4H), 2.70-2.63 (m, 1H), 2.57- 2.48 (m, 6H), 2.05-1.99 (m, 2H), 1.33 (d, 6H). LC-MS [M + H]⁺ 501.2592 |
| 430 | 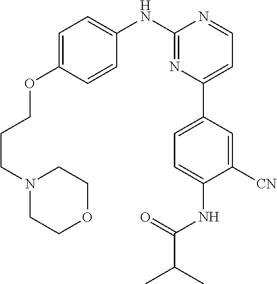 [SMILES: CC(C)C(=O)Nc1ccc(-c2ccnc(Nc3ccc(OCCCN4CCOCC4)cc3)n2)cc1C#N] | N-{2-cyano-4-[2- ({4-[3-(morpholin-4- yl)propoxy]phenyl} amino)pyrimidin-4- yl]phenyl}-2- methylpropanamide | ¹H NMR (CDCl₃) δ 8.61 (d, 1H), 8.45 (d, 1H), 8.32 (d, 1H), 8.24- 8.21 (m, 1H), 7.82 (s, 1H), 7.54- 7.49 (m, 2H), 7.26 (s, 1H), 7.05 (d, 1H), 6.95-6.92 (m, 2H), 4.05-4.02 (m, 2H), 3.75-3.72 (m, 4H), 2.70- 2.63 (m, 1H), 2.56-2.49 (m, 6H), 2.05-1.97 (m, 2H), 1.33 (d, 6H). LC-MS [M + H]⁺ 501.2586 |
| 431 | 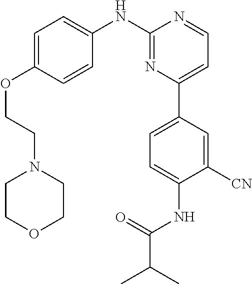 [SMILES: CC(C)C(=O)Nc1ccc(-c2ccnc(Nc3ccc(OCCN4CCOCC4)cc3)n2)cc1C#N] | N-{2-cyano-4-[2- ({4-[2-(morpholin-4- yl)ethoxy]phenyl} amino)pyrimidin-4- yl]phenyl}-2- methylpropanamide | ¹H NMR (CDCl₃) δ 8.62 (d, 1H), 8.46 (d, 1H), 8.34 (d, 1H), 8.25- 8.22 (m, 1H), 7.79 (s, 1H), 7.55- 7.51 (m, 2H), 7.15 (s, 1H), 7.06 (d, 1H), 6.96-6.93 (m, 2H), 4.15-4.12 (m, 2H), 3.77-3.75 (m, 4H), 2.85- 2.82 (m, 2H), 2.70-2.61 (m, 5H), 2.47 (s, 4H), 1.33 (d, 6H). LC-MS [M + H]⁺ 487.2440 |
| 432 | 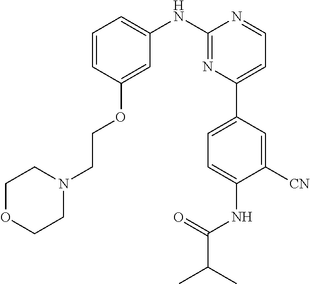 [SMILES: CC(C)C(=O)Nc1ccc(-c2ccnc(Nc3cccc(OCCN4CCOCC4)c3)n2)cc1C#N] | N-{2-cyano-4-[2- ({3-[2-(morpholin-4- yl)ethoxy]phenyl} amino)pyrimidin-4- yl]phenyl}-2- methylpropanamide | ¹H NMR (CDCl₃) δ 8.62 (d, 1H), 8.49 (d, 1H), 8.36 (d, 1H), 8.25- 8.22 (m, 1H), 7.83 (s, 1H), 7.51- 7.50 (m, 1H), 7.39 (s, 1H), 7.27- 7.23 (m, 1H), 7.12-7.10 (m, 2H), 6.65-6.62 (m, 1H), 4.18-4.16 (m, 2H), 3.75-3.73 (m, 4H), 2.87-2.85 (m, 2H), 2.70-2.60 (m, 5H), 2.47 (s, 4H), 1.33 (d, 6H). LC-MS [M + H]⁺ 487.2444 |
| 433 | 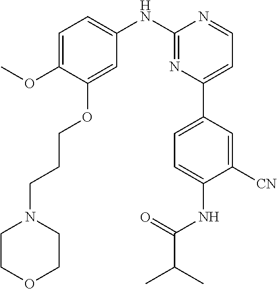 [SMILES: COc1ccc(Nc2nccc(-c3ccc(NC(=O)C(C)C)c(C#N)c3)n2)cc1OCCCN1CCOCC1] | N-{2-cyano-4-[2- ({4-methoxy-3-[3- (morpholin-4- yl)propoxy]phenyl} amino)pyrimidin-4- yl]phenyl}-2- methylpropanamide | ¹H NMR (CDCl₃) δ 8.61 (d, 1H), 8.46 (d, 1H), 8.35 (d, 1H), 8.25- 8.22 (m, 1H), 7.83 (s, 1H), 7.40 (d, 1H), 7.07-7.05 (m, 2H), 6.88 (d, 1H), 4.16-4.12 (m, 2H), 3.87 (s, 3H), 3.70-3.68 (m, 4H), 2.70-2.63 (m, 1H), 2.58-2.54 (m, 2H), 2.47 (s, 4H), 2.10-2.04 (m, 2H), 1.32 (d, 6H). LC-MS [M + H]⁺ 531.2696 |
| 434 | 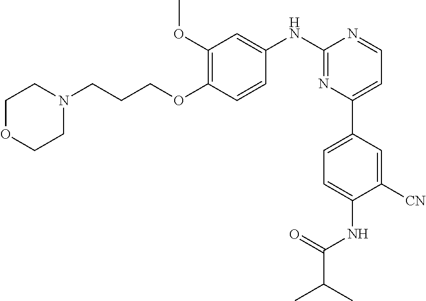 [SMILES: COc1cc(Nc2nccc(-c3ccc(NC(=O)C(C)C)c(C#N)c3)n2)ccc1OCCCN1CCOCC1] | N-{2-cyano-4-[2- ({3-methoxy-4-[3- (morpholin-4- yl)propoxy]phenyl} amino)pyrimidin-4- yl]phenyl}-2- methylpropanamide | ¹H NMR (CDCl₃) δ 8.56 (d, 1H), 8.45 (d, 1H), 8.38 (d, 1H), 8.26- 8.23 (m, 1H), 7.49 (s, 1H), 7.08 (d, 1H), 7.04-7.01 (m, 1H), 6.91 (d, 1H), 4.10-4.07 (m, 2H), 3.93 (s, 3H), 3.75-3.73 (m, 4H), 2.72-2.65 (m, 1H), 2.58-2.55 (m, 2H), 2.49 (s, 4H), 2.07-2.00 (m, 2H), 1.32 (d, 6H). LC-MS [M + H]⁺ 531.2732 |
| 435 | 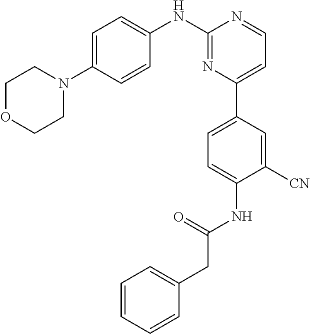 [SMILES: N#Cc1cc(-c2ccnc(Nc3ccc(N4CCOCC4)cc3)n2)ccc1NC(=O)Cc1ccccc1] | N-[2-cyano-4-(2-{[4- (morpholin-4- yl)phenyl]amino} pyrimidin-4-yl) phenyl]- 2-phenylacetamide | ¹H NMR (CDCl₃) δ 8.55-8.52 (m, 1H), 8.41 (d, 1H), 8.27-8.22 (m, 2H), 8.08 (s, 1H), 7.56-7.53 (m, 2H), 7.49-7.44 (m, 2H), 7.41-7.38 (m, 3H), 7.03 (d, 1H), 6.98-6.95 (m, 2H), 3.90-3.88 (m, 4H), 3.86 (s, 2H), 3.16-3.14 (m, 4H). LC-MS [M + H]⁺ 491.2112 |
| 436 | 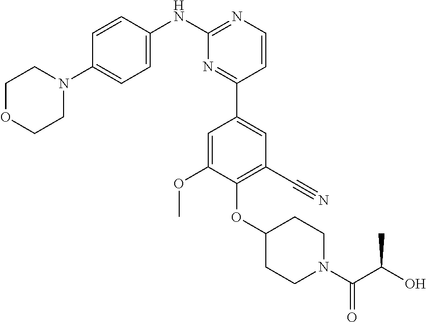 [SMILES: COc1cc(-c2ccnc(Nc3ccc(N4CCOCC4)cc3)n2)cc(C#N)c1OC1CCN(C(=O)[C@@H](C)O)CC1] | 2-({1-[(2R)-2- Hydroxypropanoyl] piperidin-4-yl}oxy)-3- methoxy-5-(2-{[4- (morpholin-4- yl)phenyl]amino} pyrimidin-4- yl)benzonitrile | ¹H NMR (DMSO-d₆) δ 9.50 (s, 1H), 8.53 (d, 1H), 8.14-8.12 (m, 2H), 7.65 (d, 2H), 7.47 (d, 1H), 6.93 (d, 2H), 4.96-4.93 (m, 1H), 4.76 (br s, 1H), 4.49-4.42 (m, 1H), 4.00 (s, 3H), 3.90-3.78 (m, 2H), 3.76-3.73 (m, 4H), 3.48-3.36 (m, 1H), 3.30- 3.26 (m, 1H), 3.05-3.03 (m, 4H), 1.99-1.84 (m, 2H), 1.78-1.62 (m, 2H), 1.19 (t, 3H). [M + H}+ LC-MS [M + H]⁺ 559.2622. |
| 437 | 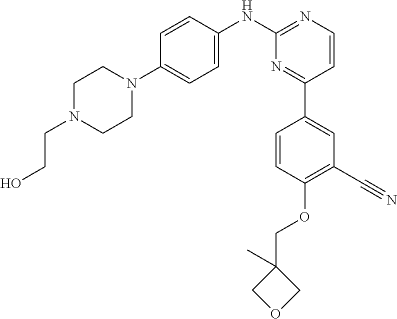 [SMILES: CC1(COc2ccc(-c3ccnc(Nc4ccc(N5CCN(CCO)CC5)cc4)n3)cc2C#N)COC1] | 5-[2-({4-[4-(2- Hydroxyethyl) piperazin-1- yl]phenyl}amino) pyrimidin-4-yl]-2-[(3- methyloxetan-3- yl)methoxy] benzonitrile | ¹H NMR (DMSO-d₆) δ 9.55 (s, 1H), 8.53-8.46 (m, 3H), 7.68 (d, 2H), 7.49 (d, 1H), 7.42 (d, 1H), 6.99 (d, 2H), 5.44 (br s, 1H), 4.54 (d, 2H), 4.36 (d, 2H), 4.35 (s, 2H), 3.81- 3.71 (m, 4H), 3.64-3.56 (m, 2H), 3.30-3.16 (m, 4H), 3.01 (t, 2H), 1.42 (s, 3H). [M + H}+ LC-MS [M + H]⁺ 501.2589. |
| 438 | 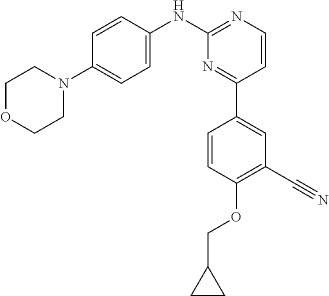 [SMILES: N#Cc1cc(-c2ccnc(Nc3ccc(N4CCOCC4)cc3)n2)ccc1OCC1CC1] | 2- (cyclopropylmethoxy)- 5-(2-{[4- (morpholin-4- yl)phenyl]amino} pyrimidin-4- yl)benzonitrile | ¹H NMR (DMSO-d₆) δ 9.48 (s, 1H), 8.51-8.42 (m, 3H), 7.65 (d, 2H), 7.40 (d, 1H), 7.39 (d, 1H), 6.95 (d, 2H), 4.11 (d, 2H), 3.77-3.74 (m, 4H), 3.08-3.05 (m, 4H), 1.35-1.28 (m, 1H), 0.65-0.60 (m, 2H), 0.42- 0.38 (m, 2H). [M + H}+ LC-MS [M + H]⁺ 428.2002. |
| 439 | 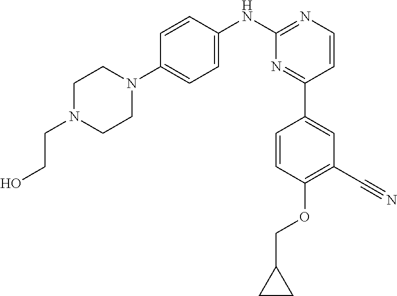 [SMILES: N#Cc1cc(-c2ccnc(Nc3ccc(N4CCN(CCO)CC4)cc3)n2)ccc1OCC1CC1] | 2- (Cyclopropylmethoxy)- 5-[2-({4-[4-(2- hydroxyethyl) piperazin-1- yl]phenyl}amino) pyrimidin-4- yl]benzonitrile | [M + H} + LC-MS [M + H]⁺ 471.2565. |
| 440 | 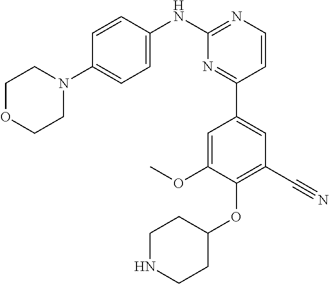 [SMILES: COc1cc(-c2ccnc(Nc3ccc(N4CCOCC4)cc3)n2)cc(C#N)c1OC1CCNCC1] | 3-Methoxy-5-(2-{[4- (morpholin-4- yl)phenyl]amino} pyrimidin-4-yl)-2- (piperidin-4- yloxy)benzonitrile | ¹H NMR (DMSO-d₆) δ 9.50 (s, 1H), 8.52 (d, 1H), 8.12-8.09 (m, 2H), 7.65 (d, 2H), 7.46 (d, 1H), 6.93 (d, 2H), 4.55-4.50 (m, 1H), 3.98 (s, 3H), 3.76-3.73 (m, 4H), 3.34 (br s, 1H), 3.05-3.03 (m, 4H), 3.10-2.97 (m, 2H), 2.48-2.44 (m, 2H), 1.89- 1.85 (m, 2H), 1.61-1.52 (m, 2H). [M + H}+ LC-MS [M + H]⁺ 487.2393. |
| 441 | 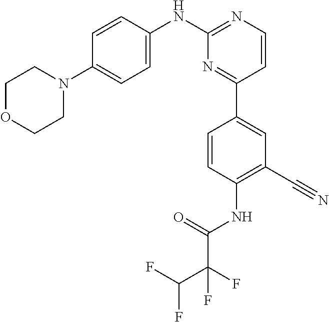 [SMILES: N#Cc1cc(-c2ccnc(Nc3ccc(N4CCOCC4)cc3)n2)ccc1NC(=O)C(F)(F)C(F)F] | N-[2-Cyano-4-(2- {[4-(morpholin-4- yl)phenyl]amino} pyrimidin-4-yl)phenyl]- 2,2,3,3- tetrafluoropropanamide | ¹H NMR (DMSO-d₆) δ 11.75 (s, 1H), 9.58 (s, 1H), 8.67-8.52 (m, 3H), 7.73-7.61 (m, 3H), 7.47 (d, 1H), 7.05-6.70 (m, 3H), 3.76-3.74 (m, 4H), 3.08-3.06 (m, 4H). A TFA salt. LC-MS [M + H]⁺ 501.1748. |
| 442 | 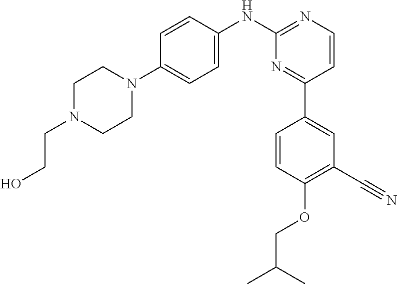 [SMILES: CC(C)COc1ccc(-c2ccnc(Nc3ccc(N4CCN(CCO)CC4)cc3)n2)cc1C#N] | 5-[2-({4-[4-(2- Hydroxyethyl) piperazin-1- yl]phenyl}amino) pyrimidin-4-yl]-2-(2- methylpropoxy) benzonitrile | ¹H NMR (DMSO-d₆) δ 9.83 (br s, 1H), 9.57 (s, 1H), 8.52-8.49 (m, 2H), 8.46-8.43 (m, 1H), 7.69 (d, 2H), 7.43-7.40 (m, 2H), 7.01 (d, 2H), 4.02 (d, 2H), 3.82-3.72 (m, 2H), 3.78-3.71 (m, 2H), 3.66-3.59 (m, 2H), 3.32-3.18 (m, 4H), 3.08- 2.99 (m, 2H), 2.16-2.06 (m, 1H), 1.04 (d, 6H), As a TFA salt. LC- MS [M + H]⁺ 473.2675. |
| 443 | 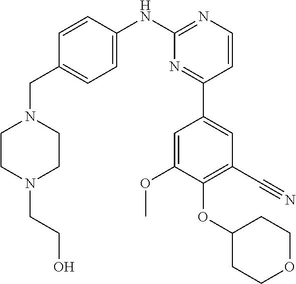 [SMILES: COc1cc(-c2ccnc(Nc3ccc(CN4CCN(CCO)CC4)cc3)n2)cc(C#N)c1OC1CCOCC1] | 5-{2-[(4-{[4-(2- Hyxdroxyethyl) piperazin-1- yl]methyl}phenyl) amino]pyrimidin-4-yl}- 3-methoxy-2- (tetrahydro-2H- pyran-4- yloxy)benzonitrile] | ¹H NMR (DMSO-d₆) δ 9.72 (s, 1H), 8.57 (d, 1H), 8.15-8.13 (m, 2H), 7.75 (d, 2H), 7.54 (d, 2H), 7.22 (d, 2H), 4.74-4.67 (m, 1H), 4.39 (br s, 1H), 4.00 (s, 3H), 3.96-3.88 (m, 2H), 3.51-3.32 (m, 8H), 2.46-2.31 (m, 8H), 1.97-1.91 (m, 2H), 1.74- 1.66 (m, 2H). LC-MS [M + H]⁺ 545.2896. |
| 444 | 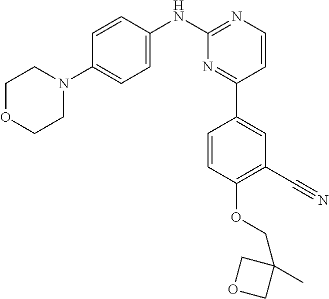 [SMILES: CC1(COc2ccc(-c3ccnc(Nc4ccc(N5CCOCC5)cc4)n3)cc2C#N)COC1] | 2-[(3-Methyloxetan- 3-yl)methoxy]-5-(2- {[4-(morpholin-4- yl)phenyl]amino} pyrimidin-4- yl)benzonitrile | LC-MS [M + H]⁺ 458.2319. |
| 445 | 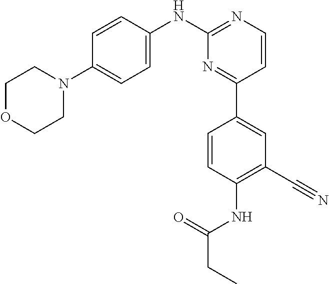 [SMILES: CCC(=O)Nc1ccc(-c2ccnc(Nc3ccc(N4CCOCC4)cc3)n2)cc1C#N] | N-[2-Cyano-4-(2- {[4-(morpholin-4- yl)phenyl]amino} pyrimidin-4- yl)phenyl] propanamide | ¹H NMR (DMSO-d₆) δ 10.30 (s, 1H), 9.51 (s, 1H), 8.55-8.52 (m, 2H), 8.45-8.42 (m, 1H), 7.85-7.81 (m, 2H), 7.42 (d, 1H), 6.94 (d, 2H), 3.76-3.73 (m, 4H), 3.06-3.04 (m, 4H), 2.47-2.42 (m, 2H), 1.12 (t, 3H). LC-MS [M + H]⁺ 429.2111. |
| 446 | 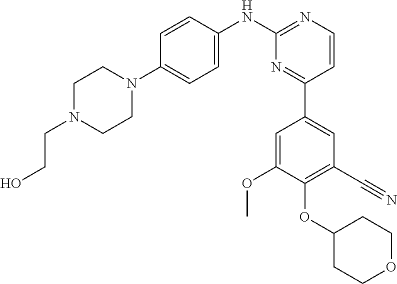 [SMILES: COc1cc(-c2ccnc(Nc3ccc(N4CCN(CCO)CC4)cc3)n2)cc(C#N)c1OC1CCOCC1] | 5-[2-({4-[4-(2- Hydroxyethyl) piperazin-1- yl]phenyl}amino) pyrimidin-4-yl]-3- methoxy-2- (tetrahydro-2H- pyran-4- yloxy)benzonitrile | ¹H NMR (DMSO-d₆) δ 9.48 (s, 1H), 8.52 (d, 1H), 8.13-8.10 (m, 2H), 7.64-7.61 (m, 2H), 7.46 (d, 1H), 6.92 (d, 2H), 4.72-4.66 (m, 1H), 3.99 (s, 3H), 3.93-3.88 (m, 2H), 3.59-3.52 (m, 2H), 3.46-3.40 (m, 2H), 3.34-3.31 (m, 2H), 3.07 (br s, 4H), 2.58 (br s, 4H), 2.51-2.49 (m, 4H), 2.46 (br s, 2H), 1.97-1.91 (m, 2H), 1.74-1.65 (m, 2H). LC-MS [M + H]⁺ 531.2748. |
| 447 | 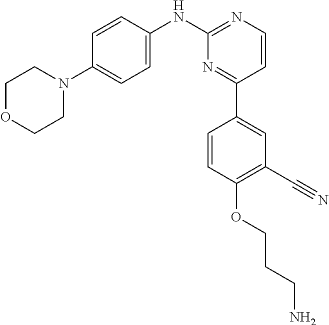 [SMILES: N#Cc1cc(-c2ccnc(Nc3ccc(N4CCOCC4)cc3)n2)ccc1OCCCN] | 2-(3- Aminopropoxy)-5- (2-{[4-(morpholin-4- yl)phenyl]amino} pyrimidin-4- yl)benzonitrile | ¹H NMR (DMSO-d₆) δ 9.52 (s, 1H), 8.54-8.47 (m, 3H), 7.86 (br s, 2H), 7.66 (d, 2H), 7.44-7.40 (m, 2H), 6.97 (d, 2H), 4.33 (t, 2H), 3.77- 3.75 (m, 4H), 3.10-3.07 (m, 4H), 3.07-3.02 (m, 2H), 2.13-2.06 (m, 2H). As a TFA salt. LC-MS [M + H]⁺ 431.2107. |
| 448 | 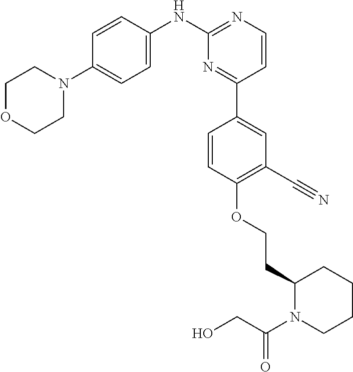 [SMILES: N#Cc1cc(-c2ccnc(Nc3ccc(N4CCOCC4)cc3)n2)ccc1OCC[C@H]1CCCCN1C(=O)CO] | 2-{2-[(2R)-1- (hydroxyacetyl) piperidin-2-yl] ethoxy}-5- (2-{[4-(morpholin-4- yl)phenyl]amino} pyrimidin-4- yl)benzonitrile | LC-MS [M + H]⁺ 543.2581 |
| 449 | 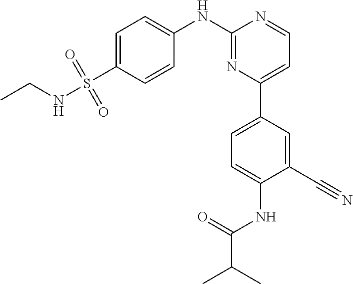 [SMILES: CCNS(=O)(=O)c1ccc(Nc2nccc(-c3ccc(NC(=O)C(C)C)c(C#N)c3)n2)cc1] | N-[2-cyano-4-(2-{[4- (ethylsulfamoyl)phenyl] amino}pyrimidin-4- yl)phenyl]-2- methylpropanamide | ¹H NMR (DMSO-d₆) δ 10.3 (s, 1H), 10.2 (s, 1H), 8.68 (d, 1H), 8.62 (d, 1H), 8.52-8.49 (m, 1H), 8.06-8.02 (m, 2H), 7.81 (d, 1H), 7.76-7.74 (m, 2H), 7.64 (d, 1H), 7.39 (t, 1H), 2.81-2.72 (m, 3H), 1.17 (d, 6H), 0.99 (t, 3H). LC-MS [M + H]⁺ 465.1673. |
| 450 | 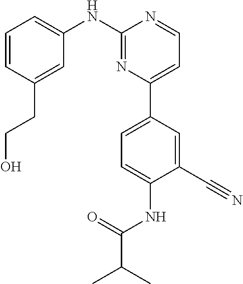 [SMILES: CC(C)C(=O)Nc1ccc(-c2ccnc(Nc3cccc(CCO)c3)n2)cc1C#N] | N-[2-cyano-4-(2-{[3- (2- hydroxyethyl)phenyl] amino}pyrimidin-4- yl)phenyl]-2- methylpropanamide | ¹H NMR (DMSO-d₆) δ 10.3 (s, 1H), 9.69 (s, 1H), 8.60-8.59 (m, 2H), 8.49-8.47 (m, 1H), 7.78 (d, 1H), 7.72 (s, 1H), 7.61-7.59 (m, 1H), 7.51 (d, 1H), 7.22 (t, 1H), 6.85 (d, 1H), 4.67 (br s, 1H), 3.66-3.62 (m, 4H), 3.56 (br s, 1H), 2.77-2.70 (m, 3H), 1.16 (d, 6H). LC-MS [M + H]⁺ 402.1380. |
| 451 | 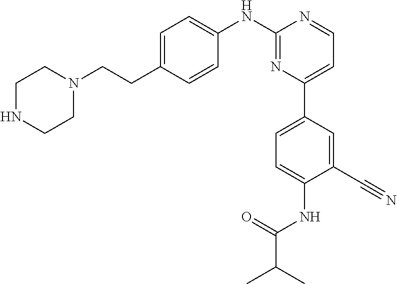 [SMILES: CC(C)C(=O)Nc1ccc(-c2ccnc(Nc3ccc(CCN4CCNCC4)cc3)n2)cc1C#N] | N-{2-cyano-4-[2- ({4-[2-(piperazin-1- yl)ethyl]phenyl} amino)pyrimidin-4- yl]phenyl}-2- methylpropanamide | LC-MS [M + H]⁺ 470.2756 |
| 452 | 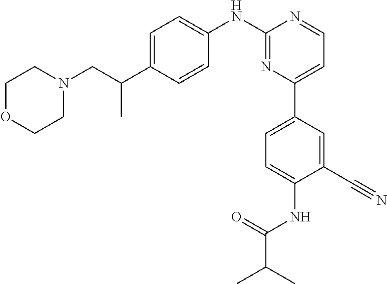 [SMILES: CC(C)C(=O)Nc1ccc(-c2ccnc(Nc3ccc(C(C)CN4CCOCC4)cc3)n2)cc1C#N] | N-{2-cyano-4-[2- ({4-[1-(morpholin-4- yl)propan-2- yl]phenyl}amino) pyrimidin-4-yl]phenyl}- 2- methylpropanamide | ¹H NMR (DMSO-d₆) δ 10.3 (s, 1H), 9.80 (s, 1H), 9.47 (br s, 1H, TFA), 8.60-8.58 (m, 2H), 8.47-8.45 (m, 1H), 7.83-7.77 (m, 3H), 7.52 (d, 1H), 7.31 (d, 2H), 3.93 (t, 2H), 3.68 (t, 2H), 3.46-3.32 (m, 4H), 3.28-3.23 (m, 1H), 3.16-3.00 (m, 2H), 2.77-2.71 (m, 1H), 1.27 (d, 3H), 1.16 (d, 6H). LC-MS [M + H]⁺ 485.2623. |
| 453 | 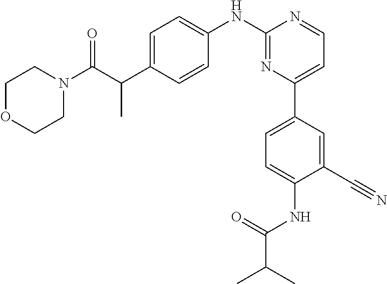 [SMILES: CC(C)C(=O)Nc1ccc(-c2ccnc(Nc3ccc(C(C)C(=O)N4CCOCC4)cc3)n2)cc1C#N] | N-{2-cyano-4-[2- ({4-[1-(morpholin-4- yl)-1-oxopropoan-2- yl]phenyl}amino) pyrimidin-4-yl] phenyl}-2- methylpropanamide | ¹H NMR (DMSO-d₆) δ 10.3 (s, 1H), 9.75 (s, 1H), 8.59-8.58 (m, 2H), 8.48-8.45 (m, 1H), 7.79-7.74 (m, 3H), 7.51 (d, 1H), 7.21 (d, 2H), 4.07-4.02 (m, 1H), 3.54-3.43 (m, 6H), 3.29-3.24 (m, 1H), 3.15-3.12 (m, 1H), 2.77-2.70 (m, 1H), 1.29 (d, 3H), 1.16 (d, 6H). LC-MS [M + H]⁺ 499.2409. |
| 454 | 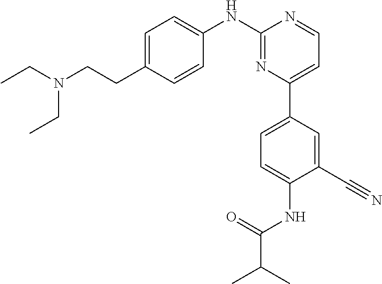 [SMILES: CCN(CC)CCc1ccc(Nc2nccc(-c3ccc(NC(=O)C(C)C)c(C#N)c3)n2)cc1] | N-{2-cyano-4-[2- ({4-[2- (diethylamino)ethyl] phenyl}amino) pyrimidin-4-yl] phenyl}-2- methylpropanamide | ¹H NMR (DMSO-d₆) δ 10.3 (s, 1H), 9.78 (s, 1H), 9.32 (br s, 1H, TFA), 8.60-8.57 (m, 2H), 8.47-8.44 (m, 1H), 7.80-7.77 (m, 3H), 7.51 (d, 1H), 7.28 (d, 2H), 3.30-3.16 (m, 6H), 2.96-2.90 (m, 2H), 2.77-2.70 (m, 1H), 1.23 (t, 6H), 1.16 (d, 6H). LC-MS [M + H]⁺ 457.2790. |
| 455 | 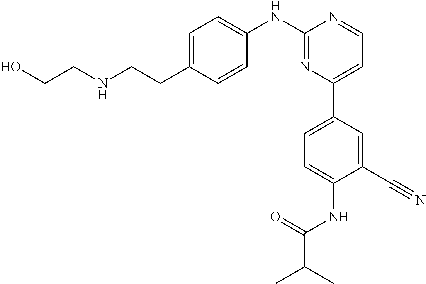 [SMILES: CC(C)C(=O)Nc1ccc(-c2ccnc(Nc3ccc(CCNCCO)cc3)n2)cc1C#N] | N-(2-cyano-4-{2-[(4- {2-[(2- hydroxyethyl)amino] ethyl}phenyl)amino] pyrimidin-4- yl}phenyl)-2- methylpropanamide | ¹H NMR (DMSO-d₆) δ 10.3 (s, 1H), 9.78 (s, 1H), 8.60-8.57 (m, 4H), 8.47-8.43 (m, 1H), 7.79-7.77 (m, 3H), 7.51 (d, 1H), 7.21 (m, 2H), 3.67 (t, 2H), 3.20-3.13 (m, 2H), 3.08-3.02 (m, 2H), 2.92-2.87 (m, 2H), 2.77-2.70 (m, 1H), 1.16 (d, 6H). LC-MS [M + H]⁺ 445.2358. |
| 456 | 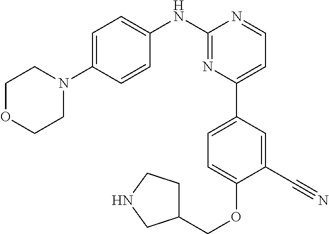 [SMILES: N#Cc1cc(-c2ccnc(Nc3ccc(N4CCOCC4)cc3)n2)ccc1OCC1CCNC1] | 5-(2-{[4-(morpholin- 4- yl)phenyl]amino} pyrimidin-4-yl)-2- (pyrrolidin-3- ylmethoxy) benzonitrile | ¹H NMR (DMSO-d₆) δ 9.51 (s, 1H), 8.84 (br s, 2H, TFA), 8.54-8.47 (m, 3H), 7.65 (d, 2H), 7.45 (d, 1H), 7.41 (d, 1H), 6.96 (d, 2H), 4.34- 4.21 (m, 2H), 3.77-3.74 (m, 4H), 3.46-3.39 (m, 1H), 3.35-3.21 (m, 2H), 3.09-3.03 (m, 5H), 2.86-2.79 (m, 1H), 2.19-2.10 (m, 1H), 1.85- 1.76 (m, 1H). LC-MS [M + H]⁺ 457.2367. |
| 457 | 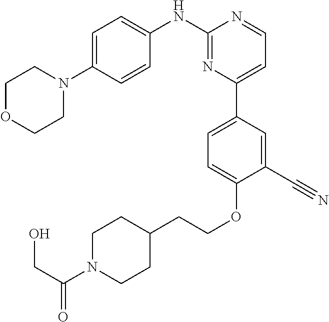 [SMILES: N#Cc1cc(-c2ccnc(Nc3ccc(N4CCOCC4)cc3)n2)ccc1OCCC1CCN(C(=O)CO)CC1] | 2-{2-[1- (hydroxyacetyl) piperidin-4-yl] ethoxy}-5- (2-{[4-(morpholin-4- yl)phenyl]amino} pyrimidin-4- yl)benzonitrile | ¹H NMR (DMSO-d₆) δ 9.46 (s, 1H), 8.51-8.44 (m, 3H), 7.63 (d, 2H), 7.45 (d, 1H), 7.39 (d, 1H), 6.92 (d, 2H), 4.47 (t, 1H), 4.36-4.29 (m, 3H), 4.07 (t, 2H), 3.69-3.64 (m, 1H), 3.06-3.03 (m, 4H), 2.94 (t, 1H), 2.62 (t, 1H), 1.76 (br s, 5H), 1.23-1.10 (m, 2H). LC-MS [M + H]⁺ 543.2723. |
| 458 | 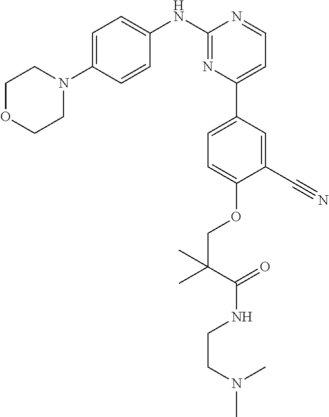 [SMILES: CN(C)CCNC(=O)C(C)(C)COc1ccc(-c2ccnc(Nc3ccc(N4CCOCC4)cc3)n2)cc1C#N] | 3-[2-cyano-4-(2-{[4- (morpholin-4- yl)phenyl]amino} pyrimidin-4- yl)phenoxy]-N-[2- (dimethylamino) ethyl]-2,2- dimethylpro- panamide | ¹H NMR (DMSO-d₆) δ 9.55 (s, 1H), 8.52-8.45 (m, 3H), 7.67 (d, 2H), 7.44 (d, 1H), 7.41 (d, 1H), 7.00 (apparent d, 2H), 4.27 (s, 2H), 3.78-3.76 (m, 4H), 3.55 (t, 2H), 3.14 (s, 6H), 3.14-3.05 (m, 4H), 2.59 (t, 2H), 1.41 (s, 6H). LC-MS [M + H]⁺ 544.2899. |
| 459 | 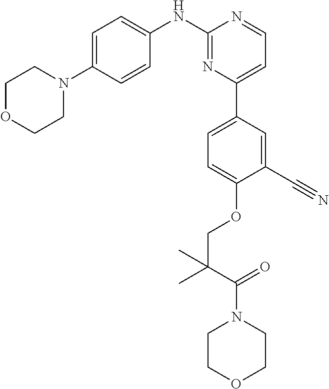 [SMILES: CC(C)(COc1ccc(-c2ccnc(Nc3ccc(N4CCOCC4)cc3)n2)cc1C#N)C(=O)N1CCOCC1] | 2-[2,2-dimethyl-3- (morpholin-4-yl)-3- oxopropoxy]-5-(2- {[4-(morpholin-4- yl)phenyl]amino} pyrimidin-4- yl)benzonitrile | ¹H NMR (DMSO-d₆) δ 9.61 (s, 1H), 8.52-8.45 (m, 3H), 7.70 (apparent d, 2H), 7.44 (d, 2H), 7.10-7.04 (m, 2H), 4.25 (s, 2H), 3.82-3.76 (m, 4H), 3.62-3.54 (m, 10H), 3.20-3.13 (m, 4H), 1.39 (s, 6H). LC-MS [M + H]⁺ 543.2714. |
| 460 | 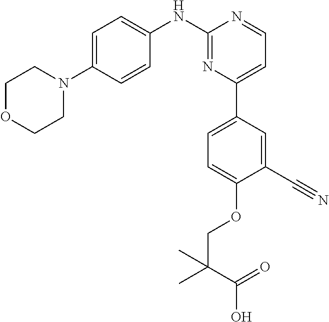 [SMILES: CC(C)(COc1ccc(-c2ccnc(Nc3ccc(N4CCOCC4)cc3)n2)cc1C#N)C(=O)O] | 3-[2-cyano-4-(2-{[4- (morpholin-4- yl)phenyl]amino} pyrimidin-4- yl)phenoxy]-2,2- dimethylpropanoic acid | ¹H NMR (DMSO-d₆) δ 9.54 (s, 1H), 8.52-8.45 (m, 3H), 7.66 (d, 2H), 7.46 (d, 1H), 7.41 (d, 1H), 7.00 (apparent d, 2H), 4.23 (s, 2H), 3.783.75 (m, 4H), 3.11 (br s, 4H), 1.28 (s, 6H). LC-MS [M + H]⁺ 474.1972. |
| 461 | 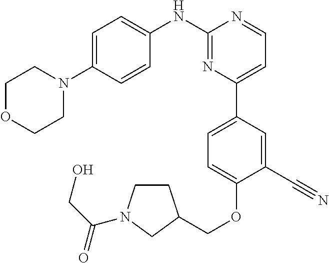 [SMILES: N#Cc1cc(-c2ccnc(Nc3ccc(N4CCOCC4)cc3)n2)ccc1OCC1CCN(C(=O)CO)C1] | 2-{[1- (hydroxyacetyl) pyrrolidin-3-yl] methoxy}- 5-(2-{[4-(morpholin- 4- yl)phenyl]amino} pyrimidin-4- yl)benzonitrile | ¹H NMR (DMSO-d₆) δ 9.56 (br s, 1H), 8.538.45 (m, 3H), 7.67 (d, 2H), 7.45 (d, 1H), 7.42 (d, 1H), 7.01 (d, 2H), 4.29-4.21 9m, 2H), 4.02-4.00 (m, 2H), 3.81-3.74 (m, 4H), 3.63-3.49 (m, 2H), 3.43-3.23 (m, 2H), 3.12 (br s, 4H), 2.83-2.65 (m, 1H), 2.18-2.03 (m, 1H), 1.91- 1.72 (m, 1H). LC-MS [M + H]⁺ 515.2274. |
| 462 | 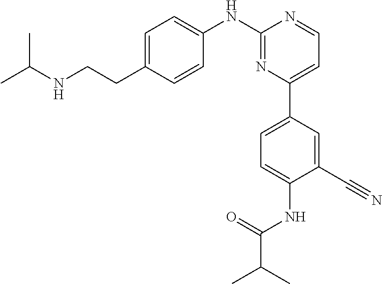 [SMILES: CC(C)NCCc1ccc(Nc2nccc(-c3ccc(NC(=O)C(C)C)c(C#N)c3)n2)cc1] | N-{2-cyano-4-[2- ({4-[2-(propan-2- ylamino)ethyl]phenyl} amino)pyrimidin-4- yl]phenyl}-2- methylpropanamide | ¹H NMR (DMSO-d₆) δ 10.3 (s, 1H), 9.77 (s, 1H), 8.60-8.57 (m, 2H), 847-8.44 (m, 1H), 8.42 (br s, 2H, TFA), 7.79-7.76 (m, 3H), 7.51 (d, 1H), 7.24 (d, 1H), 3.37-3.31 (m, 1H), 3.20-3.07 (m, 2H), 2.89-2.83 (m, 2H), 2.77-2.70 (m, 1H), 1.24 (d, 6H), 1.16 (d, 6H). LC-MS [M + H]⁺ 443.2542 |
| 463 | 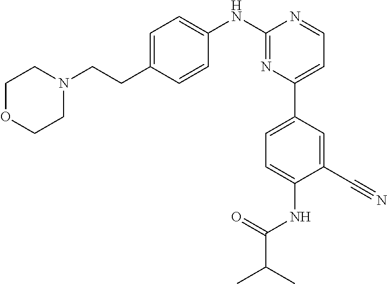 [SMILES: CC(C)C(=O)Nc1ccc(-c2ccnc(Nc3ccc(CCN4CCOCC4)cc3)n2)cc1C#N] | N-{2-cyano-4-[2- ({4-[2-(morpholin-4- yl)ethyl]phenyl} amino)pyrimidin-4- yl]phenyl}-2- methylpropanamide | ¹H NMR (DMSO-d₆) δ 10.3 (s, 1H), 9.79 (s, 1H), 8.61-8.58 (m, 2H), 8.47-8.44 (m, 1H), 7.81-7.77 (m, 3H), 7.52 (d, 1H), 7.24 (d, 2H), 4.02 (apparent d, 2H), 3.76 (t, 1H), 3.68 (t, 2H), 3.56-3.50 (m, 2H), 3.39-3.33 (m, 2H), 3.18-3.08 (m, 3H), 2.98-2.94 (m, 2H), 2.77-2.71 (m, 1H), 1.16 (d, 6H). LC-MS [M + H]⁺ 471.2359. |
| 464 | 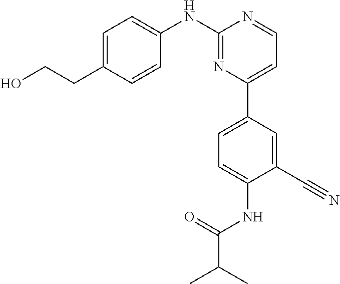 [SMILES: CC(C)C(=O)Nc1ccc(-c2ccnc(Nc3ccc(CCO)cc3)n2)cc1C#N] | N-[2-cyano-4-(2-{[4- (2- hydroxyethyl)phenyl] amino}pyrimidin-4- yl)phenyl]-2- methylpropanamide | ¹H NMR (DMSO-d₆) δ 10.3 (s, 1H), 9.67 (s, 1H), 8.58-8.56 (m, 2H), 8.47-8.44 (m, 1H), 7.78 (d, 1H), 7.70 (d, 2H), 7.48 (d, 1H), 7.16 (d, 2H), 4.64 (t, 1H), 3.61-3.56 (m, 2H), 2.77-2.67 (m, 3H), 1.16 (d, 6H). LC-MS [M + H]⁺ 402.1771. |
| 465 | 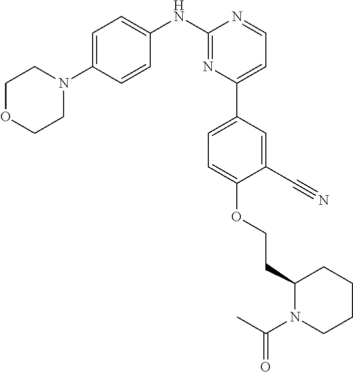 [SMILES: CC(=O)N1CCCC[C@@H]1CCOc1ccc(-c2ccnc(Nc3ccc(N4CCOCC4)cc3)n2)cc1C#N] | 2-{2-[(2R)-1- acetylpiperidin-2- yl]ethoxy}-5-(2-{[4- (morpholin-4- yl)phenyl]amino} pyrimidin-4- yl)benzonitrile | ¹H NMR (DMSO-d₆) Rotamers δ 9.47 (s, 1H), 8.528.42 (m, 3H), 7.64 (d, 2H), 7.457.32 (m, 2H), 6.93 (d, 2H), 4.37-4.10 (m, 3H), 3.77-3.73 (m, 4H), 3.06-3.04 (m, 4H)2.28-2.08 (m, 2H), 2.00 (s, 1.5H), 1.97 (s, 1.5H), 1.68-1.34 (m, 4H). LC-MS [M + H]⁺ 527.2837. |
| 466 | 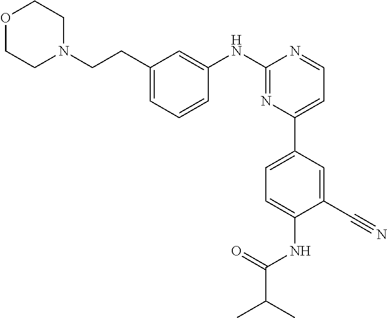 [SMILES: CC(C)C(=O)Nc1ccc(-c2ccnc(Nc3cccc(CCN4CCOCC4)c3)n2)cc1C#N] | N-{2-cyano-4-[2- ({3-[2-(morpholin-4- yl)ethyl]phenyl} amino)pyrimidin-4- yl]phenyl}-2- methylpropanamide | ¹H NMR (DMSO-d₆) δ 10.3 (s, 1H), 9.95 (br s, 1H, TFA), 9.81 (s, 21H), 8.61-8.60 (m, 2H), 8.48-8.45 (m, 1H), 7.81-7.79 (m, 2H), 7.65-7.62 (m, 1H), 7.53 (d, 1H), 7.33-7.29 (m, 1H), 6.91 (d, 1H), 4.46-4.00 (m, 2H), 3.68 (t, 2H), 3.55 (apparent d, 2H), 3.43-3.36 (m, 2H), 3.20-3.10 (m, 2H), 3.03-2.98 (m, 2H), 2.77-2.71 (m, 1H), 1.16 (d, 6H). LC-MS [M + H]⁺ 471.2543. |
| 467 | 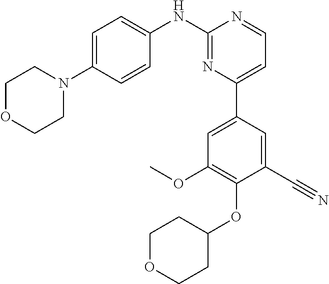 [SMILES: COc1cc(-c2ccnc(Nc3ccc(N4CCOCC4)cc3)n2)cc(C#N)c1OC1CCOCC1] | 3-methoxy-5-(2-{[4- (morpholin-4- yl)phenyl]amino} pyrimidin-4-yl)-2- (tetrahydro-2H- pyran-4- yloxy)benzonitrile | ¹H NMR (DMSO-d₆) δ 9.50 (s, 1H), 8.51 (d, 1H), 8.13-8.11 (m, 2H), 7.66-7.64 (m, 2H), 7.46 (d, 1H), 6.94-6.91 (m, 2H), 4.73-4.66 (m, 1H), 3.99 (s, 3H), 3.93-3.88 (m, 2H), 3.76-3.73 (m, 4H), 3.46-3.40 (m, 2H), 3.05-3.03 (m, 4H), 1.97- 1.92 (m, 2H), 1.74-1.65 (m, 2H). LC-MS [M + H]⁺ 488.2305. |
| 468 | 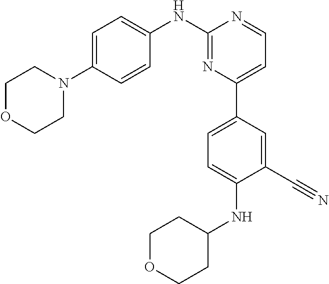 [SMILES: N#Cc1cc(-c2ccnc(Nc3ccc(N4CCOCC4)cc3)n2)ccc1NC1CCOCC1] | 5-(2-{[4-(morpholin- 4- yl)phenyl]amino} pyrimidin-4-yl)-2- (tetrahydro-2H- pyran-4- ylamino) benzonitrile | ¹H NMR (DMSO-d₆) 2:1 ration of rotamers δ 3.35 (s, 1H), 8.40 (d, 1H), 8.31 (d, 1H), 8.23-8.20 (m, 1H), 7.65-7.55 (m, 3H), 7.27 (d, 1H), 7.05 (d, 1H), 6.93-6.90 (m, 2H), 6.69 (d, 1H), 6.51 (d, 1H), 6.35 (d, 1H), 4.74 (br s, 1H), 3.91- 3.87 (m, 2H), 3.75-3.73 (m, 5H), 3.70-3.68 (m, 2H), 3.46-3.40 (m, 2H), 3.35 (s, 1H), 3.05-3.03 (m, 4H), 2.88-2.86 (m, 2H), 1.87-1.83 (m, 2H), 1.70-1.60 (m, 2H). LC- MS [M + H]⁺ 457.2355. |
| 469 | 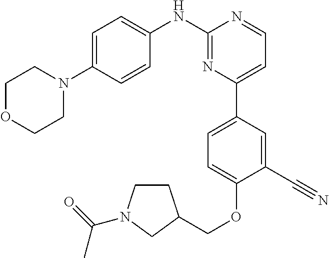 [SMILES: CC(=O)N1CCC(COc2ccc(-c3ccnc(Nc4ccc(N5CCOCC5)cc4)n3)cc2C#N)C1] | 2-[(1- acetylpyrrolidin-3- yl)methoxy]-5-(2- {[4-(morpholin-4- yl)phenyl]amino} pyrimidin-4- yl)benzonitrile | ¹H NMR (DMSO-d₆) δ 9.47 (s, 1H), 8.53-8.45 (m, 3H), 7.65-7.62 (m, 2H), 7.47-7.43 (m, 1H), 7.40-7.39 (m, 1H), 6.94-6.91 (m, 2H), 4.28- 4.20 (m, 2H), 3.76-3.72 (m, 4H), 3.70-3.45 (m, 3H), 3.22-3.17 (m, 1H), 3.06-3.03 (m, 4H), 2.83-2.65 (m, 1H), 2.16-2.00 (m, 1H), 1.95 and 1.95 (two s, 3H, rotamers ratio 5:6), 1.91-1.72 (m, 1H). LC-MS [M + H]⁺ 499.2379. |
| 470 | 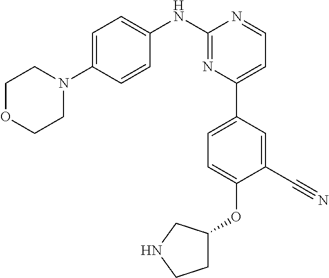 [SMILES: N#Cc1cc(-c2ccnc(Nc3ccc(N4CCOCC4)cc3)n2)ccc1O[C@@H]1CCNC1] | 5-(2-{[4-(morpholin- 4- yl)phenyl]amino} pyrimidin-4-yl)- 2-[(3R)- pyrrolidin-3- yloxy]benzonitrile | ¹H NMR (DMSO-d₆) δ 9.55 (s, 1H), 9.21 (br s, 2H, TFA), 8.57-8.46 (m, 3H), 7.67 (d, 2H), 7.50 (d, 1H), 7.43 (d, 1H), 7.00 (d, 2H), 5.45- 5.41 (m, 1H), 3.79-3.74 (m, 4H), 3.63-3.57 (m, 1H), 3.48-3.29 (m, 4H), 3.12-3.10 (m, 4H), 2.36-2.22 (m, 2H). LC-MS [M + H]⁺ 443.2174. |
| 471 | 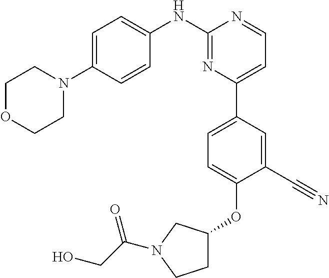 [SMILES: N#Cc1cc(-c2ccnc(Nc3ccc(N4CCOCC4)cc3)n2)ccc1O[C@@H]1CCN(C(=O)CO)C1] | 2-{[(3R)-1- (hydroxyacetyl) pyrrolidin-3-yl] oxy}-5-(2- {[4-(morpholin-4- yl)phenyl]amino} pyrimidin-4- yl)benzonitrile | ¹H NMR (DMSO-d₆) δ 9.47 (s, 1H), 8.53 (d, 1H), 8.49 (d, 1H), 8.48- 8.44 (m, 1H), 7.66-7.62 (m, 2H), 7.51 (dd, 1H), 7.40 (dd, 1H), 6.94- 6.92 (m, 2H), 5.41 (br s, 0.44H), 5.33 (br s, 0.56 H), 4.72-4.67 (m, 1H), 4.13-3.95 (m, 2H), 3.83-3.60 (m, 7H), 3.53-3.42 (m, 1H), 3.06- 3.03 (m, 4H), 2.35-2.20 (m, 1H), 2.20-2.09 (m, 1H). LC-MS [M + H]⁺ 501.2109. |
| 472 | 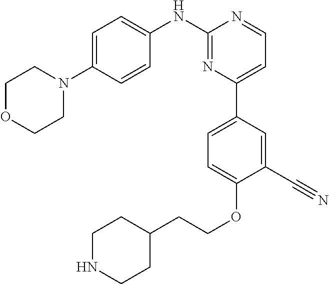 [SMILES: N#Cc1cc(-c2ccnc(Nc3ccc(N4CCOCC4)cc3)n2)ccc1OCCC1CCNCC1] | 5-(2-{[4-(morpholin- 4- yl)phenyl]amino} pyrimidin-4-yl)- 2-[2- (piperidin-4- yl)ethoxy] benzonitrile | ¹H NMR (DMSO-d₆) δ 9.52 (s, 1H), 8.60 (br s, 1H, TFA), 8.52-8.45 (m, 3H), 8.31 (br s, 1H, TFA), 7.66 (m, 2H), 7.45 (d, 1H), 7.41 (d, 1H), 6.98 (d, 2H), 4.30 (t, 2H), 3.78- 3.75 (m, 4H), 3.29 (apparent d, 2H), 3.11-3.08 (m, 4H), 2.93-2.84 (m, 2H), 1.92 (apparent d, 2H), 1.83-1.75 (m, 3H), 1.43-1.35 (m, 2H). LC-MS [M + H]⁺ 485.2762. |
| 473 | 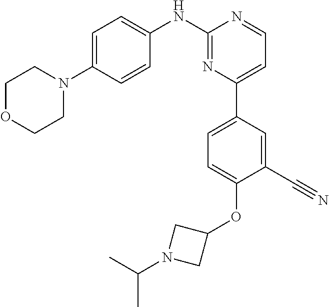 [SMILES: CC(C)N1CC(Oc2ccc(-c3ccnc(Nc4ccc(N5CCOCC5)cc4)n3)cc2C#N)C1] | 5-(2-{[4-(morpholin- 4- yl)phenyl]amino} pyrimidin-4-yl)- 2-{[1- (propan-2- yl)azetidin-3- yl]oxy}benzonitrile | ¹H NMR (CDCl₃) δ 8.44 (d, 1H), 8.32 (d, 1H), 8.22-8.19 (m, 1H), 7.55-7.52 (m, 2H), 7.05 (s, 1H), 7.01 (d, 1H), 6.98-6.95 (m, 2H), 6.84 (d, 1H), 4.94-4.91 (m, 1H), 3.97-3.92 (m, 2H), 3.90-3.87 (m, 4H), 3.24-3.18 (m, 2H), 3.16-3.13 (m, 4H), 1.01 (d, 6H). LC-MS [M + H]⁺ 471.2513. |
| 474 | 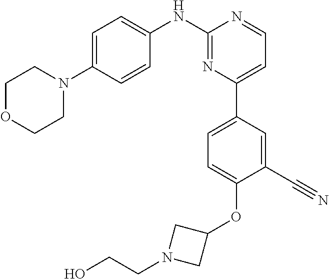 [SMILES: N#Cc1cc(-c2ccnc(Nc3ccc(N4CCOCC4)cc3)n2)ccc1OC1CN(CCO)C1] | 2-{[1-(2- hydroxyethyl)azetidin- 3-yl]oxy}-5-(2- {[4-(morpholin-4- yl)phenyl]amino} pyrimidin-4- yl)benzonitrile | ¹H NMR (CDCl₃) δ 8.44 (d, 1H), 8.32 (d, 1H), 8.23-8.20 (m, 1H), 7.56-7.52 (m, 2H), 7.11 (s, 1H), 7.01 (d, 1H), 7.00-6.95 (m, 2H), 6.82 (d, 1H), 5.01-4.97 (m, 1H), 4.01 (t, 2H), 3.90-3.87 (m, 4H), 3.62 (t, 2H), 3.36 (t, 2H), 3.16-3.14 (m, 4H), 2.78 (t, 2H). LC-MS [M + H]⁺ 473.2275. |
| 475 | 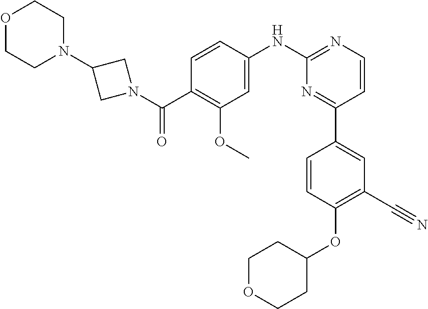 [SMILES: COc1cc(Nc2nccc(-c3ccc(OC4CCOCC4)c(C#N)c3)n2)ccc1C(=O)N1CC(N2CCOCC2)C1] | 5-{2-[(3-Methoxy-4- {[3-(morpholin-4- yl)azetidin-1- yl]carbonyl}phenyl) amino]pyrimidin-4- yl}-2-(tetrahydro- 2H-pyran-4- yloxy)benzonitrile | LC-MS [M + H]⁺ 571.2665 |
| 476 | 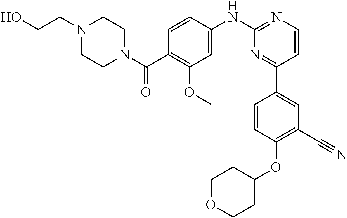 [SMILES: COc1cc(Nc2nccc(-c3ccc(OC4CCOCC4)c(C#N)c3)n2)ccc1C(=O)N1CCN(CCO)CC1] | 5-{2-[(4-{[4-(2- Hydroxyethyl) piperazin-1-yl] carbonyl}-3- methoxyphenyl) amino]pyrimidin- 4-yl}-2- (tetrahydro-2H- pyran-4- yloxy)benzonitrile | ¹H NMR (DMSO-d₆) δ 9.99 (s, 1H), 9.73 (br s, 1H), 8.61 (d, 1H), 8.60 (d, 1H), 8.46 (dd, 1H), 7.93 (br s, 1H), 7.57 (d, 1H), 7.56 (d, 1H), 7.34 (d, 1H), 7.20 (d, 1H), 5.44 (br s, 1H), 4.99-4.93 (m, 1H), 4.58 (d, 1H), 3.91-3.84 (m, 7H), 3.75 (s, 2H), 3.59-3.53 (m, 4), 3.50-3.35 (m, 2H), 3.30-3.15 (m, 2H), 3.12- 2.97 (m, 2H), 2.07-2.02 (m, 2H), 1.73-1.65 (m, 2H). LC-MS [M + H]⁺ 559.2669 |
| 477 | 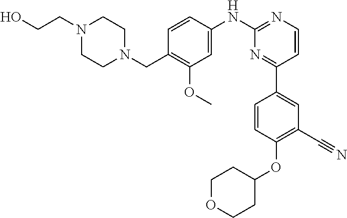 [SMILES: COc1cc(Nc2nccc(-c3ccc(OC4CCOCC4)c(C#N)c3)n2)ccc1CN1CCN(CCO)CC1] | 5-{2-[(4-{[4-(2- Hydroxyethyl) piperazin-1-yl] methyl}-3- methoxyphenyl) amino]pyrimidin- 4-yl}-2- (tetrahydro-2H- pyran-4- yloxy)benzonitrile | LC-MS [M + H]⁺ 545.2886 |
| 478 | 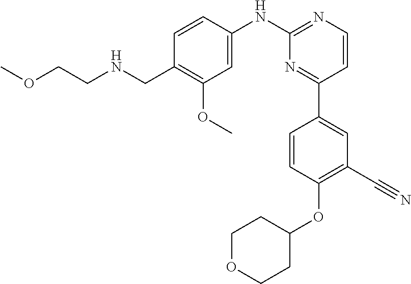 [SMILES: COCCNCc1ccc(Nc2nccc(-c3ccc(OC4CCOCC4)c(C#N)c3)n2)cc1OC] | 5-{2-[(3-Methoxy-4- {[(2- methoxyethyl)amino] methyl}phenyl)amino] pyrimidin-4-yl}-2- (tetrahydro-2H- pyran-4- yloxy)benzonitrile | ¹H NMR (DMSO-d₆) δ 9.93 (s, 1H), 8.61 (d, 1H), 8.59 (d, 1H), 8.57 (br s, 2H), 8.45 (dd, 1H), 7.89 (s, 1H), 7.56 (d, 1H), 7.54 (d, 1H), 7.32 (s, 2H), 5.00-4.93 (m, 1H), 4.08 (t, 2H), 3.91 (s, 3H), 3.89-3.84 (m, 2H), 3.60-3.49 (m, 4H), 3.32 (s, 3H), 2.08-2.02 (m, 2H), 1.73-1.65 (m, 2H). LC-MS [M + H]⁺ 490.2458 |
| 479 | 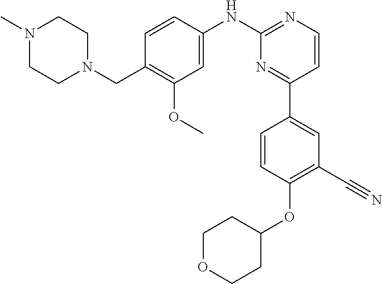 [SMILES: COc1cc(Nc2nccc(-c3ccc(OC4CCOCC4)c(C#N)c3)n2)ccc1CN1CCN(C)CC1] | 5-[2-({3-Methoxy-4- [(4-methylpiperazin- 1- yl)methyl]phenyl} amino)pyrimidin-4-yl]- 2-(tetrahydro-2H- pyran-4- yloxy)benzonitrile | LC-MS [M + H]⁺ 515.2789 |
| 480 | 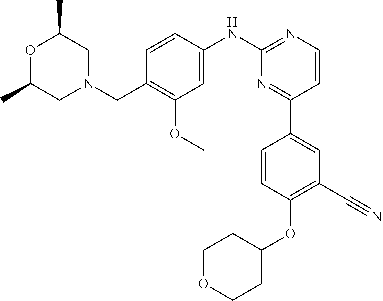 [SMILES: COc1cc(Nc2nccc(-c3ccc(OC4CCOCC4)c(C#N)c3)n2)ccc1CN1C[C@@H](C)O[C@@H](C)C1] | 5-{2-[(4-{[(2R,6S)- 2,6- Dimethylmorpholin- 4-yl]methyl}-3- methoxyphenyl)amino] pyrimidin-4-yl}-2- (tetrahydro-2H- pyran-4- yloxy)benzonitrile | ¹H NMR (DMSO-d₆) δ 10.0 (s, 1H), 9.67 (br s, 1H), 8.61 (d, 1H), 8.60 (d, 1H), 8.46 (dd, 1H), 7.94 (s, 1H), 7.56 (d, 1H), 7.55 (d, 1H), 7.36 (s, 2H), 4.99-4.93 (m, 1H), 4.23 (d, 2H), 3.93 (s, 3H), 3.91- 3.83 (m, 4H), 3.59-3.56 (m, 2H), 3.32 (d, 2H), 2.69 (q, 2H), 2.08- 2.03 (m, 2H), 1.73-1.65 (m, 2H), 1.13 (d, 6H). LC-MS [M + H]⁺ 530.2770 |
| 481 | 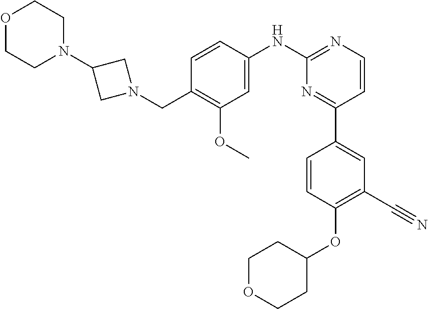 [SMILES: COc1cc(Nc2nccc(-c3ccc(OC4CCOCC4)c(C#N)c3)n2)ccc1CN1CC(N2CCOCC2)C1] | 5-{2-[(3-Methoxy-4- {[3-(morpholin-4- yl)azetidin-1- yl]methyl}phenyl) amino]pyrimidin-4-yl}- 2-(tetrahydro-2H- pyran-4- yloxy)benzonitrile | ¹H NMR (DMSO-d₆) δ 9.56 (s, 1H), 9.63 (br s, 1H), 8.61 (d, 1H), 8.59 (d, 1H), 8.45 (dd, 1H), 7.91 (s, 1H), 7.56 (d, 1H), 7.55 (d, 1H), 7.36-7.33 (m, 2H), 4.99-4.93 (m, 1H), 4.30 (s, 2H), 4.17-3.96 (m, 4H), 3.92 (s, 3H), 3.90-3.85 (m, 2H), 3.64 (br s, 4H), 3.59-3.53 (m, 2H), 3.33 (br s, 1H), 2.50-2.35 (m, 4H), 2.08-2.03 (m, 2H), 1.73-1.65 (m, 2H). LC-MS [M + H]⁺ 557.2876 |
| 482 | 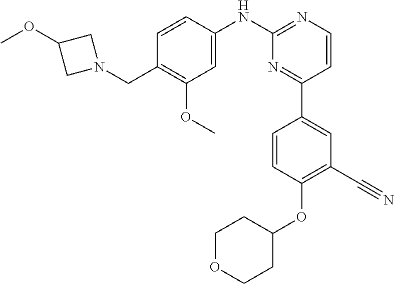 [SMILES: COc1cc(Nc2nccc(-c3ccc(OC4CCOCC4)c(C#N)c3)n2)ccc1CN1CC(OC)C1] | 5-[2-({3-Methoxy-4- [(3-methoxyazetidin- 1- yl)methyl]phenyl} amino)pyrimidin-4-yl]- 2-(tetrahydro-2H- pyran-4- yloxy)benzonitrile | ¹H NMR (DMSO-d₆) δ 9.95 (d, 1H), 9.50 (br s, 1H), 8.61 (d, 1H), 8.59 (d, 1H), 8.45 (dd, 1H), 7.90 (d, 1H), 7.56 (d, 1H), 7.55 (d, 1H), 7.36-7.31 (m, 2H), 4.99-4.93 (m, 1H), 4.32-4.23 (d, 4H), 4.21-4.16 (m, 1H), 4.02-3.85 (m, 7H), 3.59- 3.53 (m, 2H), 3.25 (s, 3H), 2.08- 2.02 (m, 2H), 1.73-1.64 (m, 2H). LC-MS [M + H]⁺ 502.2462 |
| 483 | 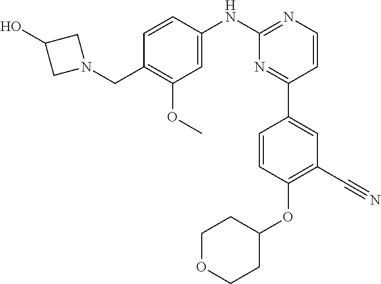 [SMILES: COc1cc(Nc2nccc(-c3ccc(OC4CCOCC4)c(C#N)c3)n2)ccc1CN1CC(O)C1] | 5-[2-({4-[(3- Hydroxyazetidin-1- yl)methyl]-3- methoxyphenyl}amino) pyrimidin-4-yl]-2- (tetrahydro-2H- pyran-4- yloxy)benzonitrile | ¹H NMR (DMSO-d₆) δ 9.96 (s, 1H), 9.31 (br s, 1H), 8.61 (d, 1H), 8.59 (d, 1H), 8.45 (dd, 1H), 7.91 (s, 1H), 7.56 (d, 1H), 7.55 (d, 1H), 7.33 (s, 2H), 6.20 (d, 1H), 4.99- 4.93 (m, 1H), 4.42-4.38 (m, 1H), 4.28 (d, 2H), 4.25-4.17 (m, 2H), 3.92 (s, 3H), 3.90-3.85 (m, 4H), 3.59-3.53 (m, 2H), 2.08-2.02 (m, 2H), 1.72-1.65 (m, 2H). LC-MS [M + H]⁺ 488.2297 |
| 484 | 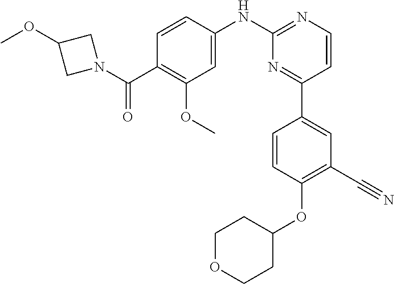 [SMILES: COc1cc(Nc2nccc(-c3ccc(OC4CCOCC4)c(C#N)c3)n2)ccc1C(=O)N1CC(OC)C1] | 5-[2-({3-Methoxy-4- [(3-methoxyazetidin- 1- yl)carbonyl]phenyl} amino)pyrimidin-4- yl]-2-(tetrahydro-2H- pyran-4- yloxy)benzonitrile | ¹H NMR (DMSO-d₆) δ 9.95 (s, 1H), 8.61 (d, 1H), 8.59 (d, 1H), 8.47 (dd, 1H), 7.84 (s, 1H), 7.57- 7.53 (m, 2H), 7.33 (dd, 1H), 7.25 (d, 1H), 5.72 (br s, 1H), 4.99-4.93 (m, 1H), 4.23-4.13 (m, 2H), 4.08- 4.02 (m, 1H), 3.90-3.85 (m, 2H), 3.88 (s, 3H), 3.79-3.75 (m, 2H), 3.59-3.53 (m, 2H), 3.20 (s, 3H), 2.08-2.02 (m, 2H), 1.73-1.65 (m, 2H). LC-MS [M + H]⁺ 516.2242 |
| 485 | 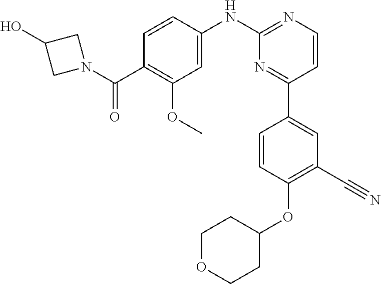 [SMILES: COc1cc(Nc2nccc(-c3ccc(OC4CCOCC4)c(C#N)c3)n2)ccc1C(=O)N1CC(O)C1] | 5-[2-({4-[(3- Hydroxyazetidinb-1- yl)carbonyl]-3- methoxyphenyl} amino)pyrimidin- 4-yl]-2-(tetrahydro- 2H-pyran-4- yloxy)benzonitrile | ¹H NMR (DMSO-d₆) δ 9.94 (s, 1H), 8.62 (d, 1H), 8.59 (d, 1H), 8.46 (dd, 1H), 7.85 (s, 1H), 7.57- 7.53 (m, 2H), 7.32 (dd, 1H), 7.24 (d, 1H), 5.72 (br s, 1H), 4.99-4.93 (m, 1H), 4.49-4.43 (m, 1H), 4.17 (dd, 1H), 4.05 (dd, 1H), 3.90-3.85 (m, 2H), 3.88 (s, 3H), 3.74-3.68 (m, 2H), 3.59-3.53 (m, 2H), 2.69 (q, 2H), 2.08-2.02 (m, 2H), 1.73-1.65 (m, 2H). LC-MS [M + H]⁺ 502.2087 |
| 486 | 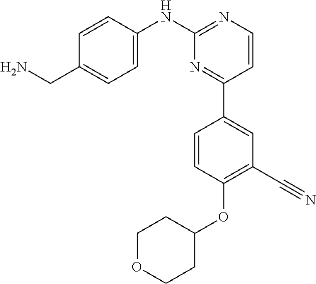 [SMILES: N#Cc1cc(-c2ccnc(Nc3ccc(CN)cc3)n2)ccc1OC1CCOCC1] | 5-(2-{[4- (aminomethyl)phenyl] amino}pyrimidin-4- yl)-2-(tetrahydro-2H- pyran-4- yloxy)benzonitrile | ¹H NMR (DMSO-d₆) δ 9.85 (s, 1H), 8.58 (d, 1H), 8.55 (d, 1H), 8.45 (dd, 1H), 8.05 (br s, 2H), 7.85 (d, 2H), 7.55 (d, 1H), 7.51 (d, 1H), 7.40 (d, 2H), 4.98-4.94 (m, 1H), 4.01-3.96 (m, 2H), 3.90-3.85 (m, 2H), 3.59-3.53 (m, 2H), 2.08-2.00 (m, 2H), 1.73-1.65 (m, 2H). LC- MS [M + H]⁺ 402.1927 |
| 487 | 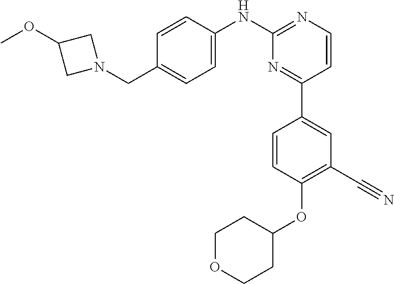 [SMILES: COC1CN(Cc2ccc(Nc3nccc(-c4ccc(OC5CCOCC5)c(C#N)c4)n3)cc2)C1] | 5-[2-({4-[(3- methoxyazetidin-1- ylmethyl]phenyl} amino)pyrimidin- 4-yl]- 2-(tetrahydro-2H- pyran-4- yloxy)benzonitrile | ¹H NMR (DMSO-d₆) δ 9.91 (s, 1H), 8.80 (br s, 1H), 8.59 (d, 1H), 8.55 (d, 1H), 8.44 (dd, 1H), 7.88 (d, 2H), 7.54 (t, 2H), 7.42 (br s, 2H), 4.99-4.93 (m, 1H), 4.31 (br s, 2H), 4.23 (br s, 3H), 3.95 (br s, 2H), 3.90-3.85 (m, 2H), 3.59-3.53 (m, 2H), 3.25 (s, 3H), 2.08-2.02 (m, 2H), 1.72-1.65 (m, 2H). LC-MS [M + H]⁺ 472.2347 |
| 488 | 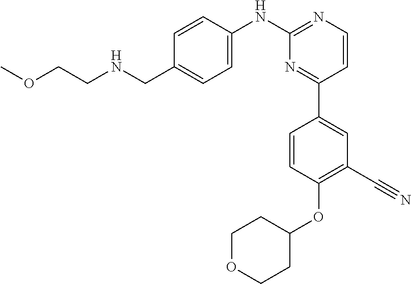 [SMILES: COCCNCc1ccc(Nc2nccc(-c3ccc(OC4CCOCC4)c(C#N)c3)n2)cc1] | 5-{2-[(4-{[(2- methoxyethyl)amino] methyl}phenyl)amino] pyrimidin-4-yl}-2- (tetrahydro-2H- pyran-4- yloxy)benzonitrile | ¹H NMR (DMSO-d₆) δ 9.89 (s, 1H), 8.80 (br s, 1H), 8.59 (d, 1H), 8.55 (d, 1H), 8.45 (dd, 1H), 7.77 (d, 2H), 7.55 (d, 1H), 7.52 (d, 1H), 7.43 (d, 2H), 4.99-4.93 (m, 1H), 4.11 (s, 2H), 3.90-3.85 (m, 2H), 3.59-3.53 (m, 4H), 3.35 (s, 3H), 3.09 (br s, 2H), 2.08-2.02 (m, 2H), 1.73-1.65 (m, 2H). LC-MS [M + H]⁺ 460.2345 |
| 489 | 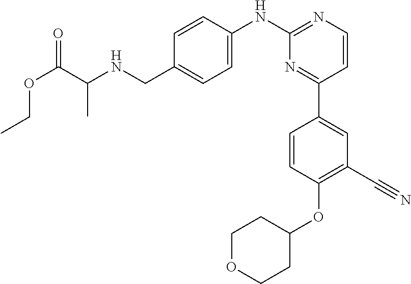 [SMILES: CCOC(=O)C(C)NCc1ccc(Nc2nccc(-c3ccc(OC4CCOCC4)c(C#N)c3)n2)cc1] | ethyl N-[4-({4-[3- cyano-4-(tetrahydro- 2H-pyran-4- yloxy)phenyl] pyrimidin-2- yl}amino)benzyl] alaninate | ¹H NMR (DMSO-d₆) δ 9.91 (s, 1H), 9.41 (br s, 1H), 9.32 (br s, 1H), 8.59 (d, 1H), 8.56 (d, 1H), 8.45 (dd, 1H), 7.88 (d, 2H), 7.55 (d, 1H), 7.54 (d, 1H), 7.43 (d, 2H), 4.99-4.93 (m, 1H), 4.24 (q, 2H), 4.18-4.08 (m, 3H), 3.90-3.85 (m, 2H), 3.59-3.53 (m, 2H), 2.08-2.02 (m, 2H), 1.73-1.65 (m, 2H), 1.50 (d, 3H), 1.26 (t, 3H). LC-MS [M + H]⁺ 502.2447 |
| 490 | 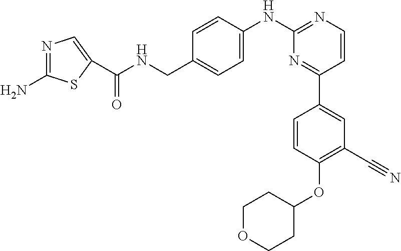 [SMILES: N#Cc1cc(-c2ccnc(Nc3ccc(CNC(=O)c4cnc(N)s4)cc3)n2)ccc1OC1CCOCC1] | 2-amino-N-[4-({4-[3- cyano-4-(tetrahydro- 2H-pyran-4- yloxy)phenyl] pyrimidin-2- yl}amino)benzyl]- 1,3-thiazole-5- carboxamide | ¹H NMR (DMSO-d₆) δ 9.69 (s, 1H), 8.61 (t, 2H), 8.54 (s, 1H), 8.53 (d, 1H), 8.45 (dd, 1H), 7.75 (d, 2H), 7.57 (d, 1H), 7.66 (s, 1H), 7.56 (d, 1H), 7.47 (s, 2H), 7.46 (s, 1H), 7.23 (d, 2H), 4.98-4.92 (m, 1H), 4.34 (d, 2H), 3.90-3.85 (m, 2H), 3.58-3.52 (m, 2H), 2.08-2.02 (m, 2H), 1.73-1.64 (m, 2H). LC- MS [M + H]⁺ 528.1807 |
| 491 | 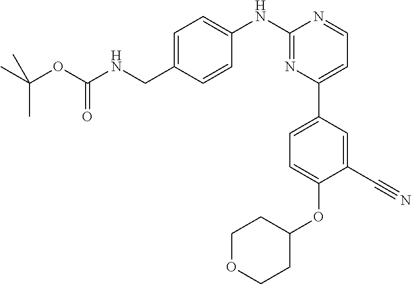 [SMILES: CC(C)(C)OC(=O)NCc1ccc(Nc2nccc(-c3ccc(OC4CCOCC4)c(C#N)c3)n2)cc1] | tert-butyl [4-({4-[3- cyano-4-(tetrahydro- 2H-pyran-4- yloxy)phenyl] pyrimidin-2- yl}amino)benzyl] carbamate | ¹H NMR (DMSO-d₆) δ 9.67 (s, 1H), 8.55 (s, 1H), 8.53 (d, 1H), 8.45 (dd, 1H), 7.72 (d, 2H), 7.56 (d, 1H), 7.46 (d, 1H), 7.36 (t, 1H), 7.18 (d, 2H), 4.99-4.92 (m, 1H), 4.08 (d, 2H), 3.90-3.85 (m, 2H), 2.58-2.55 (m, 2H), 2.08-2.02 (m, 2H), 1.73-1.65 (m, 2H), 1.40 (s, 9H). LC-MS [M + H]⁺ 502.2446 |
| 492 | 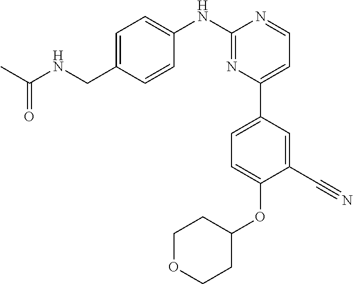 [SMILES: CC(=O)NCc1ccc(Nc2nccc(-c3ccc(OC4CCOCC4)c(C#N)c3)n2)cc1] | N-[4-({4-[3-cyano-4- (tetrahydro-2H- pyran-4- yloxy)phenyl] pyrimidin-2- yl}amino)benzyl] acetamide | ¹H NMR (DMSO-d₆) δ 9.69 (s, 1H), 8.55-8.53 (m, 2H), 8.45 (dd, 1H), 8.31 (t, 1H), 7.74 (d, 2H), 7.57 (d, 1H), 7.47 (d, 1H), 7.20 (d, 2H), 4.99-4.92 (m, 1H), 4.20 (d, 2H), 3.90-3.85 (m, 2H), 3.58-3.53 (m, 2H), 2.08-2.02 (m, 2H), 1.86 (s, 3H), 1.73-1.64 (m, 2H). LC-MS [M + H]⁺ 444.2030 |
| 493 | 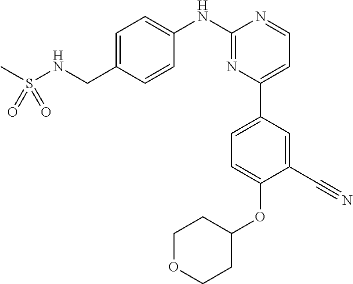 [SMILES: CS(=O)(=O)NCc1ccc(Nc2nccc(-c3ccc(OC4CCOCC4)c(C#N)c3)n2)cc1] | N-[4-({4-[3-cyano-4- (tetrahydro-2H- pyran-4- yloxy)phenyl] pyrimidin-2- yl}amino)benzyl] methanesulfonamide | ¹H NMR (DMSO-d₆) δ 9.74 (s, 1H), 8.56-8.54 (m, 2H), 8.46 (dd, 1H), 7.78 (d, 2H), 7.57 (d, 1H), 7.51 (t, 1H), 7.48 (d, 1H), 7.29 (d, 2H), 4.99-4.92 (m, 1H), 4.11 (d, 2H), 3.90-3.85 (m, 2H), 3.58-3.53 (m, 2H), 2.08-2.01 (m, 2H), 1.73- 1.65 (m, 2H). LC-MS [M + H]⁺ 480.1685 |
| 494 | 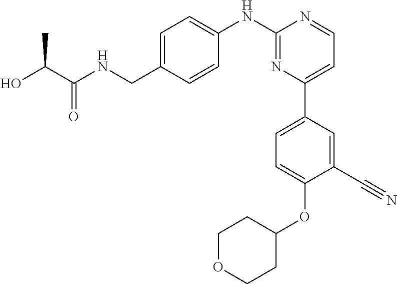 [SMILES: C[C@H](O)C(=O)NCc1ccc(Nc2nccc(-c3ccc(OC4CCOCC4)c(C#N)c3)n2)cc1] | (2S)-N-[4-({4-[3- cyano-4-(tetrahydro- 2H-pyran-4- yloxy)phenyl] pyrimidin-2- yl}amino)benzyl]-2- hydroxypropanamide | ¹H NMR (DMSO-d₆) δ 9.68 (s, 1H), 8.55-8.53 (m, 2H), 8.45 (dd, 1H), 8.17 (t, 1H), 7.72 (d, 2H), 7.55 (d, 1H), 7.46 (d, 1H), 7.20 (d, 2H), 4.99-4.92 (m, 1H), 4.23 (d, 2H), 4.01 (q, 1H), 3.90-3.85 (m, 2H), 3.58-3.564 (m, 2H), 2.08-2.01 (m, 2H), 1.73-1.65 (m, 2H). LC- MS [M + H]⁺ 474.2126 |
| 495 | 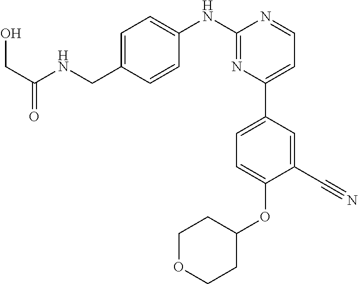 [SMILES: N#Cc1cc(-c2ccnc(Nc3ccc(CNC(=O)CO)cc3)n2)ccc1OC1CCOCC1] | N-[4-({4-[3-cyano-4- (tetrahydro-2H- pyran-4- yloxy)phenyl] pyrimidin-2- yl}amino)benzyl]-2- hydroxyacetamide | ¹H NMR (DMSO-d₆) δ 9.68 (s, 1H), 8.55 (s, 1H), 8.53 (d, 1H), 8.45 (dd, 1H), 8.22 (t, 1H), 7.73 (d, 2H), 7.57 (d, 1H), 7.46 (d, 1H), 7.22 (d, 2H), 4.98-4.91 (m, 1H), 4.26 (d, 2H), 3.90-3.85 (m, 2H), 3.85 (s, 2H), 3.58-3.53 (m, 2H), 2.08-2.01 (m, 2H), 1.72-1.65 (m, 2H). LC-MS [M + H]⁺ 460.1962 |
| 496 | 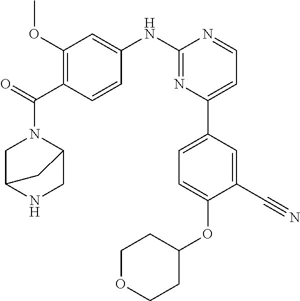 [SMILES: COc1cc(Nc2nccc(-c3ccc(OC4CCOCC4)c(C#N)c3)n2)ccc1C(=O)N1CC2CC1CN2] | 5-(2-{[4-(2,5- diazabicyclo[2.2.1] hept-2-ylcarbonyl)-3- methoxyphenyl] amino} pyrimidin-4-yl)-2- (tetrahydro-2H- pyran-4- yloxy)benzonitrile | ¹H NMR (DMSO-d₆) δ 9.98 (d, 1H), 8.62-8.59 (m, 2H), 8.47 (dd, 1H), 7.92 (s, 1H), 7.57-7.54 (m, 2H), 7.34 (dt, 1H), 7.21 (d, 1H), 5.10-4.93 (m, 1H), 4.10 (d, 1H), 3.90-3.85 (m, 2H), 3.89 (s, 3H), 3.59-3.52 (m, 2H), 3.48 (t, 1H), 3.39-3.31 (m, 2H), 3.17-3.08 (m, 2H), 2.09-2.02 (m, 2H), 1.96-1.80 (m, 2H), 1.73-1.65 (m, 2H). LC- MS [M + H]⁺ 527.2399 |
| 497 | 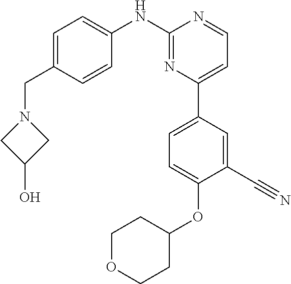 [SMILES: N#Cc1cc(-c2ccnc(Nc3ccc(CN4CC(O)C4)cc3)n2)ccc1OC1CCOCC1] | 5-[2-({4-[(3- hydroxyazetidin-1- yl)methyl]phenyl} amino)pyrimidin- 4-yl]- 2-(tetrahydro-2H- pyran-4- yloxy)benzonitrile | ¹H NMR (DMSO-d₆) δ 9.92 (s, 1H), 9.71 (br s, 1H), 8.59 (d, 1H), 8.55 (d, 1H), 8.45 (dd, 1H), 7.88 (d, 2H), 7.54 (dd, 2H), 7.47-7.44 (m, 1H), 7.42 (d, 1H), 5.09 (t, 1H), 4.98-4.90 (m, 1H), 4.45 (d, 2H), 3.90-3.85 (m, 2H), 3.58-3.52 (m, 2H), 2.08-2.00 (m, 2H), 1.73-1.65 (m, 2H). LC-MS [M + H]⁺ 458.2168 |
| 498 | 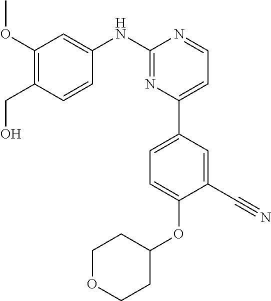 [SMILES: COc1cc(Nc2nccc(-c3ccc(OC4CCOCC4)c(C#N)c3)n2)ccc1CO] | 5-(2-{[4- (hydroxymethyl)-3- methoxyphenyl] amino} pyrimidin-4-yl)-2- (tetrahydro-2H- pyran-4- yloxy)benzonitrile | ¹H NMR (DMSO-d₆) δ 9.69 (s, 1H), 8.58 (d, 1H), 8.56 (d, 1H), 8.46 (dd, 1H), 7.68 (s, 1H), 7.56 (d, 1H), 7.47 (d, 1H), 7.26 (t, 2H), 4.98-4.93 (m, 1H), 4.87 (t, 1H), 4.45 (d, 1H), 3.90-3.85 (m, 2H), 3.82 (s, 3H), 3.58-3.53 (m, 2H), 2.06-1.98 (m, 2H), 1.73-1.64 (m, 2H). LC-MS [M + H]⁺ 433.1835 |
| 499 | 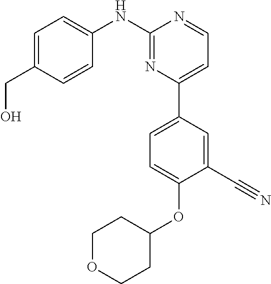 [SMILES: N#Cc1cc(-c2ccnc(Nc3ccc(CO)cc3)n2)ccc1OC1CCOCC1] | 5-(2-{[4- (hydroxymethyl) phenyl] amino}pyrimidin- 4-yl)-2-(tetrahydro- 2H-pyran-4- yloxy)benzonitrile | ¹H NMR (DMSO-d₆) δ 9.68 (s, 1H), 8.55 (s, 1H), 8.54 (d, 1H), 8.46 (dd, 1H), 7.74 (d, 2H), 7.56 (d, 1H), 7.46 (d, 1H), 7.26 (d, 2H), 5.09 (t, 1H), 4.98-4.90 (m, 1H), 4.45 (d, 2H), 3.90-3.85 (m, 2H), 3.58-3.52 (m, 2H), 2.08-2.00 (m, 2H), 1.73-1.65 (m, 2H). LC-MS [M + H]⁺ 403.1760 |
| 500 | 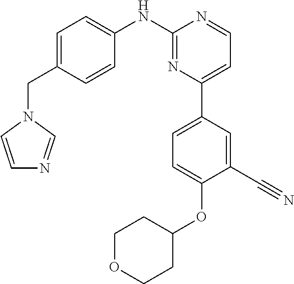 [SMILES: N#Cc1cc(-c2ccnc(Nc3ccc(Cn4ccnc4)cc3)n2)ccc1OC1CCOCC1] | 5-(2-{[4-(1H- imidazol-1- ylmethyl)phenyl] amino}pyrimidin- 4-yl)- 2-(tetrahydro-2H- pyran-4- yloxy)benzonitrile | ¹H NMR (DMSO-d₆) δ 9.89 (s, 1H), 9.33 (t, 1H), 8.57 (d, 1H), 8.55 (d, 1H), 8.45 (dd, 1H), 7.85 (d, 2H), 7.81 (t, 1H), 7.11 (t, 1H), 7.56 (d, 1H), 7.52 (d, 1H), 7.41 (d, 2H), 5.39 (s, 2H), 4.99-4.93 (m, 1H), 3.90-3.85 (m, 2H), 3.89 (s, 23H), 3.59-3.53 (m, 2H), 2.07-2.02 (m, 2H), 1.73-1.65 (m, 2H). LC- MS [M + H]⁺ 453.2009 |
| 501 | 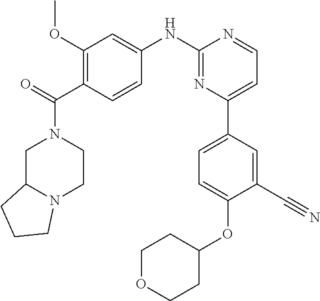 [SMILES: COc1cc(Nc2nccc(-c3ccc(OC4CCOCC4)c(C#N)c3)n2)ccc1C(=O)N1CCN2CCCC2C1] | 5-(2-{[4- (hexahydropyrrolo[1, 2-a]pyrazin-2(1H)- ylcarbonyl)-3- methoxyphenyl] amino} pyrimidin-4-yl)-2- (tetrahydro-2H- pyran-4- yloxy)benzonitrile | LC-MS [M + H]⁺ 555.2689 |
| 502 | 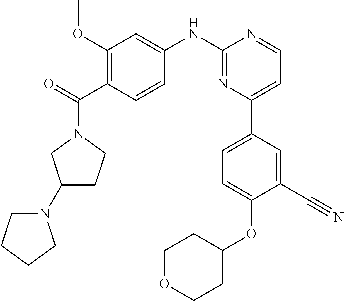 [SMILES: COc1cc(Nc2nccc(-c3ccc(OC4CCOCC4)c(C#N)c3)n2)ccc1C(=O)N1CCC(N2CCCC2)C1] | 5-(2-{[4-(1,3′- bipyrrolidin-1′- ylcarbonyl)-3- methoxyphenyl] amino} pyrimidin-4-yl)-2- (tetrahydro-2H- pyran-4- yloxy)benzonitrile | ¹H NMR (DMSO-d₆) δ 10.1 (brr s, 1H), 9.96 (d, 1H), 8.62-8.59 (m, 2H), 8.46 (dd, 1H), 7.91 (d, 1H), 7.57-7.54 (m, 2H), 7.33 (d, 1H), 7.18 (dd, 1H), 4.99-4.93 (m, 1H), 3.98-3.84 (m, 6H), 3.70-3.53 (m, 5H), 3.51-3.38 (m, 2H), 3.31 (q, 1H), 3.18-3.05 (m, 2H), 2.08-2.00 (m, 4H), 1.90-1.80 (m, 2H), 1.73- 1.65 (m, 2H). LC-MS [M + H]⁺ 569.2853 |
| 503 | 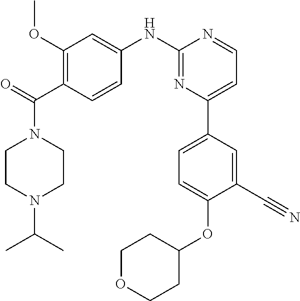 [SMILES: COc1cc(Nc2nccc(-c3ccc(OC4CCOCC4)c(C#N)c3)n2)ccc1C(=O)N1CCN(C(C)C)CC1] | 5-{2-[(3-methoxy-4- {[4-(propan-2- yl)piperazin-1- yl]carbonyl}phenyl) amino]pyrimidin-4- yl}-2-(tetrahydro- 2H-pyran-4- yloxy)benzonitrile | ¹H NMR (DMSO-d₆) δ 9.99 (br s, 1H), 9.57 (br s, 1H), 8.62 (d, 1H), 8.60 (d, 1H), 8.46 (dd, 1H), 7.92 (s, 1H), 7.58 (d, 1H), 7.55 (s, 1H), 7.35 (d, 1H), 7.23 (br s, 1H), 4.99- 4.93 (m, 1H), 4.66 (d, 1H), 3.89 (s, 3H), 3.89-3.84 (m, 2H), 3.62-3.53 (m, 4H), 3.48-3.22 (m, 2H), 3.13- 3.06 (m, 2H), 3.06-2.88 (m, 2H), 2.07-2.03 (m, 2H), 1.73-1.65 (m, 2H), 1.27 (d, 6H). LC-MS [M + H]⁺ 557.2851 |
| 504 | 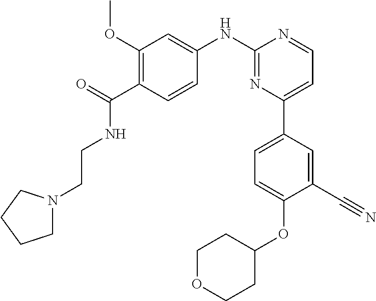 [SMILES: COc1cc(Nc2nccc(-c3ccc(OC4CCOCC4)c(C#N)c3)n2)ccc1C(=O)NCCN1CCCC1] | 4-({4-[3-cyano-4- (tetrahydro-2H- pyran-4- yloxy)phenyl] pyrimidin- 2-yl}amino)-2- methoxy-N-[2- (pyrrolidin-1- yl)ethyl]benzamide | ¹H NMR (DMSO-d₆) δ 10.6 (br s, 1H), 10.1 (br s, 1H), 8.63 (d, 1H), 8.61 (d, 1H), 8.48-8.44 (m, 2H), 7.97 (s, 1H), 7.86 (d, 1H), 7.59 (s, 1H), 7.57 (d, 1H), 7.38 (dd, 1H), 5.00-4.94 (m, 1H), 4.00 (s, 3H), 3.91-3.85 (m, 2H), 3.69-3.59 (m, 4H), 3.59-3.53 (m, 2H), 3.01 (q, 2H), 3.06-3.98 (m, 2H), 2.09-1.98 (m, 4H), 1.90-1.85 (m, 2H), 1.73- 1.65 (m, 2H). LC-MS [M + H]⁺ 543.2682 |
| 505 | 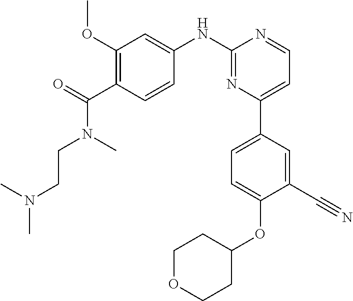 [SMILES: COc1cc(Nc2nccc(-c3ccc(OC4CCOCC4)c(C#N)c3)n2)ccc1C(=O)N(C)CCN(C)C] | 4-({4-[3-cyano-4- (tetrahydro-2H- pyran-4- yloxy)phenyl] pyrimidin-2-yl} amino)-N-[2- (dimethylamino) ethyl]-2-mrthoxy-N- methylbenzamide | ¹H NMR (DMSO-d₆) δ 9.95 (brr s, 1H), 9.46 (br s, 1H), 8.62-8.59 (m, 2H), 8.46 (dd, 1H), 7.88 (s, 1H), 7.57-7.54 (m, 2H), 7.34-7.34 (d, 1H), 7.17 (d, 1H), 4.99-4.94 (m, 1H), 3.87 (s, 6H), 3.80-3.76 (m, 2H), 3.35-3.33 (m, 2H), 2.90 (t, 6H), 2.71-2.60 (m, 2H), 1.76-1.65 (m, 2H). LC-MS [M + H]⁺ 531.2736 |
| 506 | 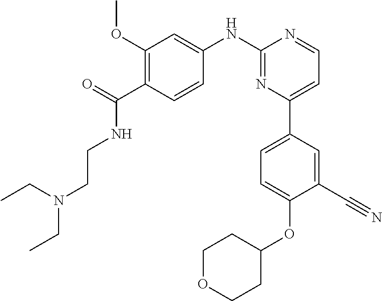 [SMILES: CCN(CC)CCNC(=O)c1ccc(Nc2nccc(-c3ccc(OC4CCOCC4)c(C#N)c3)n2)cc1OC] | 4-({4-[3-cyano-4- (tetrahydro-2H- pyran-4- yloxy)phenyl] pyrimidin-2-yl} amino)-N-[2- (diethylamino)ethyl]- 2-methoxybenzamide | ¹H NMR (DMSO-d₆) δ 10.1 (br s, 1H), 9.16 (d, 1H), 8.67-8.61 (m, 2H), 8.48-8.41 (m, 2H), 7.99 (s, 1H), 7.87 (d, 1H), 7.60-7.55 (m, 2H), 7.38 (d, 1H), 5.00-4.91 (m, 1H), 4.01 (s, 3H), 3.90-3.85 (m, 2H), 3.67-3.52 (m, 4H), 3.27-3.18 (m, 6H), 2.09-1.99 (m, 2H), 1.74- 1.66 (m, 2H), 1.22 (t, 6H). LC-MS [M + H]⁺ 545.2912 |
| 507 | 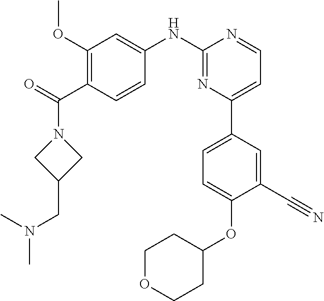 [SMILES: COc1cc(Nc2nccc(-c3ccc(OC4CCOCC4)c(C#N)c3)n2)ccc1C(=O)N1CC(CN(C)C)C1] | 5-(2-{[4-({3- [(dimethylamino) methyl]azetidin-1- yl}carbonyl)-3- methoxyphenyl] amino} pyrimidin-4-yl)-2- (tetrahydro-2H- pyran-4- yloxy)benzonitrile | ¹H NMR (DMSO-d₆) δ 9.98 (br s, 1H), 9.42 (br s, 1H), 8.62 (d, 1H), 8.60 (d, 1H), 8.46 (dd, 1H), 7.88 (s, 1H), 7.57-7.55 (m, 2H), 7.34-7.26 (m, 2H), 4.99-4.94 (m, 1H), 4.11 (q, 1H), 3.90 (s, 3H), 3.90-3.85 (m, 2H), 3.81 (dd, 1H), 3.76 (dd, 1H), 3.59-3.53 (m, 2H), 3.42-3.30 (m, 2H), 3.08-3.00 (m, 1H), 2.75 (t, 6H), 2.06-2.02 (m, 2H), 1.73-1.65 (m, 2H). LC-MS [M + H]⁺ 543.2751 |
| 508 | 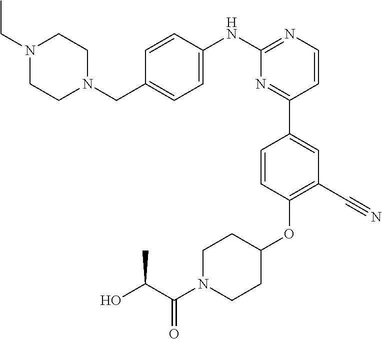 [SMILES: CCN1CCN(Cc2ccc(Nc3nccc(-c4ccc(OC5CCN(C(=O)[C@H](C)O)CC5)c(C#N)c4)n3)cc2)CC1] | 5-[2-({4-[(4- ethylpiperazin-1- yl)methyl]phenyl} amino)pyrimidin- 4-yl]-2-({1-[(2S)-2- hydroxypropanoyl] piperidin-4- yl}oxy)benzonitrile | ¹H NMR (MeOH-d₄) δ 8.52-8.50 (m, 2H), 8.43-8.40 (m, 1H), 7.92 (d, 1H), 7.50-7.47 (m, 2H), 7.43- 7.40 (m, 1H), 7.40 (d, 2H), 4.83- 4.62 (m, 2H), 4.33 (m, 1H), 4.10- 4.00 (m, 4H), 3.83-3.70 (m, 4H), 3.42-3.40 (m, 3H), 3.25-3.20 (m, 4H), 2.12-2.00 (m, 4H), 1.40 (s, 3H), 1.30 (s, 3H). LC-MS [M + H]⁺ 570.3038 |
| 509 | 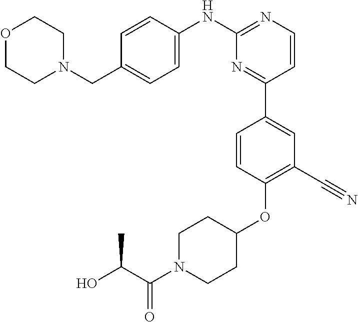 [SMILES: C[C@H](O)C(=O)N1CCC(Oc2ccc(-c3ccnc(Nc4ccc(CN5CCOCC5)cc4)n3)cc2C#N)CC1] | 2-({1-[(2S)-2- hydroxypropanoyl] piperidin-4-yl}oxy)-5- (2-{[4-(morpholin-4- ylmethyl)phenyl] amino}pyrimidin-4- yl)benzonitrile | ¹H NMR (MeOH-d₄) δ 8.50-8.47 (m, 2H), 8.43-8.40 (m, 1H), 7.80 (d, 1H), 7.42-7.40 (m, 2H), 7.33 (d, 1H), 7.30 (d, 2H), 4.64-4.60 (m, 2H), 3.80-3.74 (m, 4H), 3.70-3.67 (m, 2H), 3.18-3.12 (m, 4H), 2.10- 2.00 (m, 4H), 1.95-1.87 (m, 4H), 1.34 (s, 3H). LC-MS [M + H]⁺ 543.2712 |
| 510 | 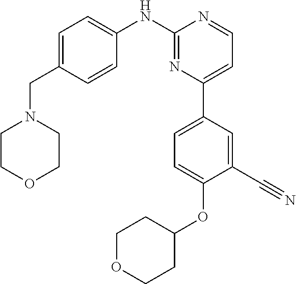 [SMILES: N#Cc1cc(-c2ccnc(Nc3ccc(CN4CCOCC4)cc3)n2)ccc1OC1CCOCC1] | 5-(2-{[4-(morpholin- 4- ylmethyl)phenyl] amino}pyrimidin-4-yl)- 2-(tetrahydro-2H- pyran-4- yloxy)benzonitrile | ¹H NMR (MeOH-d₄) δ 8.50-8.45 (m, 2H), 8.42-8.40 (m, 1H), 7.70 (d, 2H), 7.40 (d, 1H), 7.31-7.30 (m, 3H), 4.02-3.97 (m, 2H), 3.71-3.63 (m, 6H), 3.50 (s, 2H), 2.50-2.47 (m, 5H), 2.14-2.08 (m, 2H), 1.90-1.80 (m, 2H). LC-MS [M + H]⁺ 472.2310 |
| 511 | 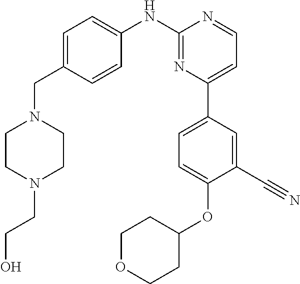 [SMILES: N#Cc1cc(-c2ccnc(Nc3ccc(CN4CCN(CCO)CC4)cc3)n2)ccc1OC1CCOCC1] | 5-{2-[(4-{[4-(2- hydroxyethyl) piperazin-1- yl]methyl}phenyl) amino]pyrimidin-4-yl}- 2-(tetrahydro-2H- pyran-4- yloxy)benzonitrile | ¹H NMR (MeOH-d₄) δ 8.50-8.49 (m, 2H), 8.41-8.40 (m, 1H), 7.81 (d, 1H), 7.40 (d, 2H), 7.38 (s, 1H), 7.35-7.33 (m, 2H), 4.04-4.03 (m, 2H), 4.00-3.97 (m, 5H), 3.84-3.81 (m, 4H), 3.70-3.64 (m, 4H), 3.20- 3.12 (m, 4H), 2.14-2.10 (m, 2H), 1.90-1.80 (m, 2H). LC-MS [M + H]⁺ 515.2780 |
| 512 | 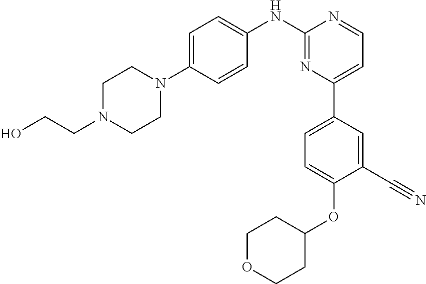 [SMILES: N#Cc1cc(-c2ccnc(Nc3ccc(N4CCN(CCO)CC4)cc3)n2)ccc1OC1CCOCC1] | 5-[2-({4-[4-(2- hydroxyethyl) piperazin-1- yl]phenyl}amino) pyrimidin-4-yl]-2- (tetrahydro-2H- pyran-4- yloxy)benzonitrile | ¹H NMR (MeOH-d₄) δ 8.47-8.46 (m, 2H), 8.41-8.40 (m, 1H), 8.39- 8.38 (m, 1H), 7.62-7.60 (m, 1H), 7.40-7.37 (m, 1H), 7.32-7.30 (m, 1H), 7.10-7.00 (m, 2H), 4.04-3.97 (m, 2H), 3.95-3.92 (m, 2H), 3.80- 3.72 (m, 4H), 3.70-3.63 (m, 3H), 3.40-3.34 (m, 4H), 3.20-3.12 (m, 2H), 2.14-2.08 (m, 2H), 1.90-1.80 (m, 2H). LC-MS [M + H]⁺ 501.2618 |
| 513 | 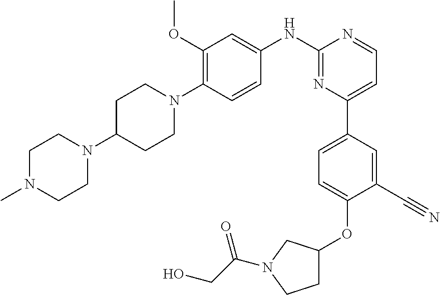 [SMILES: COc1cc(Nc2nccc(-c3ccc(OC4CCN(C(=O)CO)C4)c(C#N)c3)n2)ccc1N1CCC(N2CCN(C)CC2)CC1] | 2-{[1- (hydroxyacetyl) pyrrolidin-3-yl] oxy}-5-[2- ({3-methoxy-4-[4-(4- methylpiperazin-1- yl)piperidin-1- yl]phenyl}amino) pyrimidin-4- yl]benzonitrile | ¹H NMR (MeOH-d₄) δ 8.50-8.48 (m, 1H), 8.45-8.44 (m, 1H), 8.43- 8.42 (m, 1H), 8.41-8.40 (m, 1H), 8.38-8.36 (m, 1H), 7.40-7.37 (m, 1H), 7.20-7.14 (m, 1H), 7.14-7.12 (m, 1H), 4.30-4.24 (m, 2H), 4.20- 4.18 (m, 2H), 4.00 (bs, 2H), 3.90- 3.82 (m, 2H), 3.80-3.77 (m, 2H), 3.53-3.40 (m, 4H), 3.30-3.10 (m, 4H), 3.00 (s, 3H), 2.41-2.36 (m, 4H), 2.30-2.22 (m, 3H), 2.10-2.04 (m, 4H). LC-MS [M + H]⁺ 627.3415 |
| 514 | 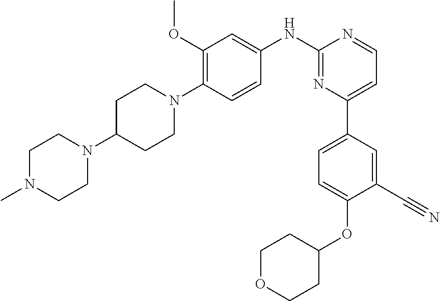 [SMILES: COc1cc(Nc2nccc(-c3ccc(OC4CCOCC4)c(C#N)c3)n2)ccc1N1CCC(N2CCN(C)CC2)CC1] | 5-[2-({3-methoxy-4- [4-(4- methylpiperazin-1- yl)piperidin-1- yl]phenyl}amino) pyrimidin-4-yl]-2- (tetrahydro-2H- pyran-4- yloxy)benzonitrile | ¹H NMR (MeOH-d₄) δ 8.50-8.48 (m, 1H), 8.45-8.44 (m, 1H), 8.43- 8.40 (m, 1H), 8.38-8.36 (m, 1H), 7.44-7.43 (m, 1H), 7.42-7.40 (m, 1H), 7.2 (bs, 1H), 7.14-7.12 (m, 1H), 4.00 (s, 3H), 3.98-3.97 (m, 2H), 3.86-3.83 (m, 2H), 3.70-3.64 (m, 2H), 3.54-3.48 (m, 4H), 3.41 (m, 4H), 3.13-3.10 (m, 2H), 2.92 (s, 3H), 2.26-2.23 (m, 4H), 2.15- 2.10 (m, 2H), 2.06-2.03 (m, 2H), 1.90-1.80 (m, 2H). LC-MS [M + H]⁺ 584.3344 |
| 515 | 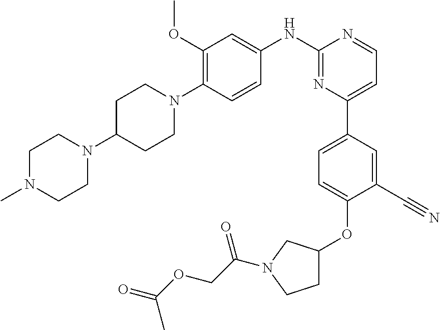 [SMILES: COc1cc(Nc2nccc(-c3ccc(OC4CCN(C(=O)COC(C)=O)C4)c(C#N)c3)n2)ccc1N1CCC(N2CCN(C)CC2)CC1] | 2-(3-{2-cyano-4-[2- ({3-methoxy-4-[4-(4- methylpiperazin-1- yl)piperidin-1- yl]phenyl}amino) pyrimidin-4- yl]phenyoxy} pyrrolidin- 1-yl)-2-oxoethyl acetate | LC-MS [M + H]⁺ 669.410 |
| 516 | 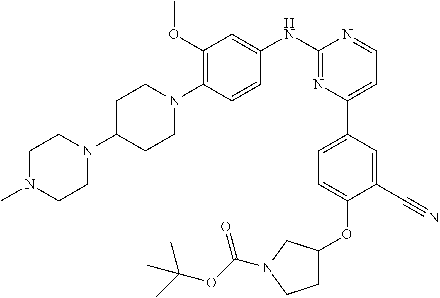 [SMILES: COc1cc(Nc2nccc(-c3ccc(OC4CCN(C(=O)OC(C)(C)C)C4)c(C#N)c3)n2)ccc1N1CCC(N2CCN(C)CC2)CC1] | tert-butyl 3-{2- cyano-4-[2-({3- methoxy-4-[4-(4- methylpiperazin-1- yl)piperidin-1- yl]phenyl}amino) pyrimidin-4- yl]phenoxy} pyrrolidine- 1-carboxylate | LC-MS [M + H]⁺ 669.500 |
| 517 | 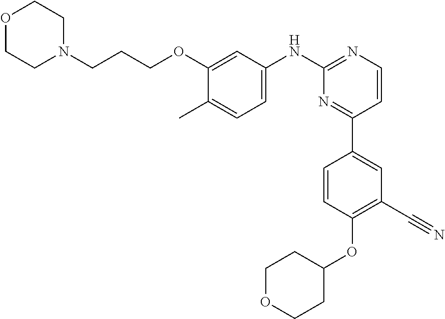 [SMILES: Cc1ccc(Nc2nccc(-c3ccc(OC4CCOCC4)c(C#N)c3)n2)cc1OCCCN1CCOCC1] | 5-[2-({4-Methyl-3- [3-(morpholin-4- yl)propoxy]phenyl} amino)pyrimidin-4- yl]-2-(tetrahydro-2H- pyran-4- yloxy)benzonitrile | ¹H NMR (DMSO-d₆) δ 9.79 (br s, 1H), 8.65 (s, 1H), 8.57 (d, 1H), 8.55 (d, 1H), 8.44 (dd, 1H), 7.65 (s, 1H), 7.55 (d, 1H), 7.47 (d, 1H), 7.20 (d, 1H), 7.07 (d, 1H), 4.96 (sept., 1H), 4.11 (t, 2H), 4.02 (d, 2H), 3.93-3.84 (m, 2H), 3.66 (t, 2H), 3.62-3.44 (m, 4H), 3.40-3.30 (m, 2H), 3.20-3.17 (m, 2H), 2.28- 2.16 (m, 2H), 2.15 (s, 3H), 2.10- 2.00 (m, 2H), 1.75-1.62 (m, 2H). LC-MS [M + H]⁺ 530.2769. |
| 518 | 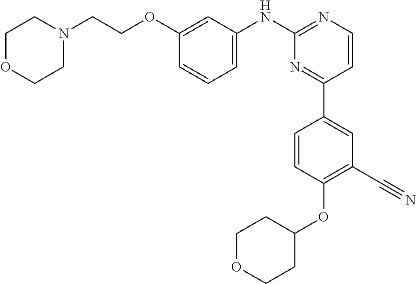 [SMILES: N#Cc1cc(-c2ccnc(Nc3cccc(OCCN4CCOCC4)c3)n2)ccc1OC1CCOCC1] | 5-[2-({3-[2- (Morpholin-4- yl)ethoxy]phenyl} amino)pyrimidin- 4-yl]- 2-(tetrahydro-2H- pyran-4- yloxy)benzonitrile | ¹H NMR (DMSO-d₆) δ 9.97 (br s, 1H), 9.80 (s, 1H), 8.58 (d, 1H), 8.57 (d, 1H), 8.45 (dd, 1H), 7.70 (t, 1H), 7.55 (d, 1H), 7.51 (d, 1H), 7.37 (d, 1H), 7.26 (t, 1H), 6.65 (dd, 1H), 4.95 (sept., 1H), 4.38 (t, 2H), 4.00 (d, 2H), 3.93-3.82 (m, 2H), 3.71 (t, 2H), 3.65-3.45 (m, 6H), 3.30-3.15 (m, 2H), 2.10-1.98 (m, 2H), 1.78-1.60 (m, 2H). LC-MS [M + H]⁺ 502.2450. |
| 519 | 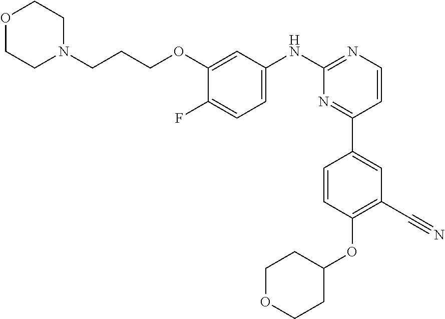 [SMILES: N#Cc1cc(-c2ccnc(Nc3ccc(F)c(OCCCN4CCOCC4)c3)n2)ccc1OC1CCOCC1] | 5-[2-({4-Fluoro-3-[3- (morpholin-4- yl)propoxy]phenyl} amino)pyrimidin-4- yl]-2-(tetrahydro-2H- pyran-4- yloxy)benzonitrile | ¹H NMR (DMSO-d₆) δ 9.76 (s, 1H), 9.70 (br s, 1H), 8.56 (dd, 2H), 8.47 (dd, 1H), 7.85 (d, 1H), 7.55 (d, 1H), 7.50 (d, 1H), 7.29-7.22 (m, 1H), 7.18 (d, 1H), 4.96 (sept., 1H), 4.19 (t, 2H), 4.01 (d, 2H), 3.94- 3.84 (m, 2H), 3.65 (t, 2H), 3.57 (ddd, 2H), 3.55-3.46 (m, 2H), 3.39- 3.29 (m, 2H), 3.18-3.06 (m, 2H), 2.28-2.16 (m, 2H), 2.10-1.99 (m, 2H), 1.75-1.62 (m, 2H). LC-MS [M + H]⁺ 534.2519. |
| 520 | 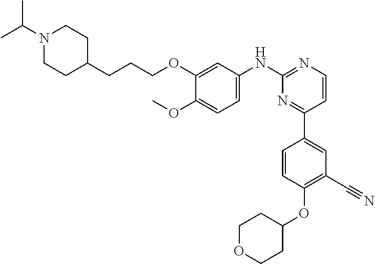 [SMILES: COc1ccc(Nc2nccc(-c3ccc(OC4CCOCC4)c(C#N)c3)n2)cc1OCCCC1CCN(C(C)C)CC1] | 5-{2-[(4-medthoxy-3- {3-[1-(propan-2- yl)piperidin-4- yl]propoxy}phenyl) amino]pyrimidin-4- yl}-2-(tetrahydro- 2H-pyran-4- yloxy)benzonitrile | ¹H NMR (DMSO-d₆) δ 9.53 (s, 1H), 8.78 (br s, 1H), 8.54 (d, 1H), 8.52 (d, 1H), 8.43 (dd, 1H), 7.63 (br s, 1H), 7.54 (d, 1H), 7.42 (d, 1H), 7.19 (d, 1H), 6.92 (d, 1H), 4.95 (sept., 1H), 3.98 (t, 2H), 3.92-3.84 (m, 2H), 3.74 (s, 3H), 3.56 (ddd, 2H), 3.48-3.40 (m, 1H), 3.35 (d, 2H), 2.91 (q, 2H), 2.10-2.00 (m, 2H), 1.91 (d, 2H), 1.84-1.74 (m, 2H), 1.74-1.62 (m, 2H), 1.58 (br s, 1H), 1.42-1.28 (m, 4H), 1.23 (d, 6H). LC-MS [M + H]⁺ 586.3392. |
| 521 | 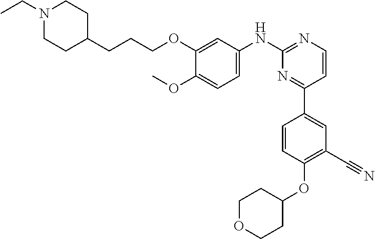 [SMILES: CCN1CCC(CCCOc2cc(Nc3nccc(-c4ccc(OC5CCOCC5)c(C#N)c4)n3)ccc2OC)CC1] | 5-[2-({3-[3-(1- ethylpiperidin-4- yl)propoxy]-4- methoxyphenyl} amino) pyrimidin-4-yl]-2- (tetrahydro-2H- pyran-4- yloxy)benzonitrile | ¹H NMR (DMSO-d₆) δ 9.53 (s, 1H), 8.91 (br s, 1H), 8.53 (d, 1H), 8.52 (d, 1H), 8.43 (dd, 1H), 7.63 (s, 1H), 7.54 (d, 1H), 7.42 (d, 1H), 7.20 (d, 1H), 6.91 (d, 1H), 4.95 (sept., 1H), 3.98 (t, 2H), 3.92-3.83 (m, 2H), 3.74 (s, 3H), 3.56 (ddd, 2H), 3.49 (d, 2H), 3.13-3.03 (m, 2H), 2.83 (q, 2H), 2.10-2.00 (m, 2H), 1.91 (d, 2H), 1.83-1.74 (m, 2H), 1.74-1.62 (m, 2H), 1.56 (br s, 1H), 1.45-1.35 (m, 2H), 1.35-1.25 (m, 2H), 1.21 (t, 3H). LC-MS [M + H]⁺ 572.3234. |
| 522 | 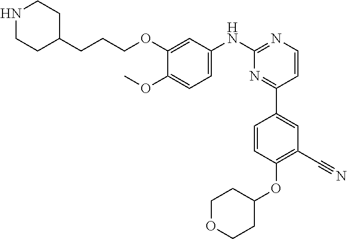 [SMILES: COc1ccc(Nc2nccc(-c3ccc(OC4CCOCC4)c(C#N)c3)n2)cc1OCCCC1CCNCC1] | 5-[2-({4-methoxy-3- [3-(piperidin-4- yl)propoxy]phenyl} amino)pyrimidin-4- yl]-2-(tetrahydro-2H- pyran-4- yloxy)benzonitrile | ¹H NMR (DMSO-d₆) δ 9.52 (s, 1H), 8.53 (d, 1H), 8.52 (d, 1H), 8.43 (dd, 1H), 8.18 (br s, 1H), 7.63 (s, 1H), 7.54 (d, 1H), 7.42 (d, 1H), 7.20 (dd, 1H), 6.91 (d, 1H), 4.95 (sept., 1H), 3.98 (t, 2H), 3.92-3.82 (m, 2H), 3.74 (s, 3H), 3.56 (ddd, 2H), 3.26 (d, 2H), 2.83 (q, 2H), 2.10-1.98 (m, 2H), 1.88-1.75 (m, 4H), 1.75-1.62 (m, 2H), 1.62-1.50 (m, 1H), 1.45-1.32 (m, 2H), 1.32- 1.18 (m, 2H). LC-MS [M + H]⁺ 544.2925. |
| 523 | 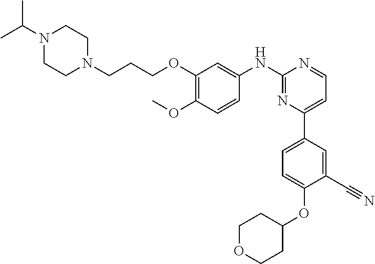 [SMILES: COc1ccc(Nc2nccc(-c3ccc(OC4CCOCC4)c(C#N)c3)n2)cc1OCCCN1CCN(C(C)C)CC1] | 5-{2-[(4-methoxy-3- {3-[4-(propan-2- yl)piperazin-1- yl]propoxy}phenyl) amino]pyrimidin-4- yl}-2-(tetrahydro- 2H-pyran-4- yloxy)benzonitrile | ¹H NMR (DMSO-d₆) δ 9.56 (s, 1H), 8.55 (d, 1H), 8.52 (d, 1H), 8.43 (dd, 1H), 7.64 (br s, 1H), 7.55 (d, 1H), 7.44 (d, 1H), 7.24 (dd, 1H), 6.95 (d, 1H), 4.95 (sept., 1H), 4.09 (t, 2H), 3.92-3.83 (m, 2H), 3.76 (s, 3H), 3.56 (ddd, 2H), 3.75-3.48 (m, 5H), 3.28-2.80 (m, 6H), 2.20-2.10- (m, 2H), 2.08-1.98 (m, 2H), 1.74- 1.60 (m, 2H), 1.25 (d, 6H). LC-MS [M + H]⁺ 587.3343. |
| 524 | 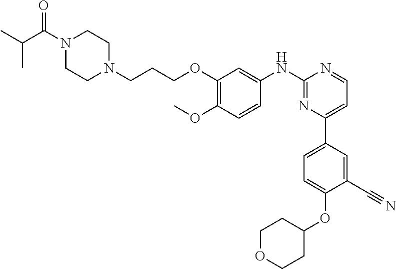 [SMILES: COc1ccc(Nc2nccc(-c3ccc(OC4CCOCC4)c(C#N)c3)n2)cc1OCCCN1CCN(C(=O)C(C)C)CC1] | 5-{2-[(4-methoxy-3- {3-[4-(2- methylpropanoyl) piperazin-1- yl]propoxy}phenyl) amino]pyrimidin-4- yl}-2-(tetrahydro- 2H-pyran-4- yloxy)benzonitrile | ¹H NMR (DMSO-d₆) δ 9.56 (br s, 2H), 8.56 (d, 1H), 8.52 (d, 1H), 8.43 (d, 1H), 7.64 (br s, 1H), 7.55 (d, 1H), 7.44 (d, 1H), 7.25 (dd, 1H), 6.95 (d, 1H), 4.95 (sept., 1H), 4.58-4.44 (m, 1H), 4.25-4.12 (m, 1H), 4.10 (t, 2H), 3.93-3.82 (m, 2H), 3.76 (s, 3H), 3.62-3.50 (m, 4H), 3.45-3.25 (m, 3H), 3.18-3.00 (m, 1H), 3.00-2.82 (m, 3H), 2.28- 2.14 (m, 2H), 2.10-1.98 (m, 2H), 1.74-1.60 (m, 2H), 1.02 (br s, 6H). LC-MS [M + H]⁺ 615.3276. |
| 525 | 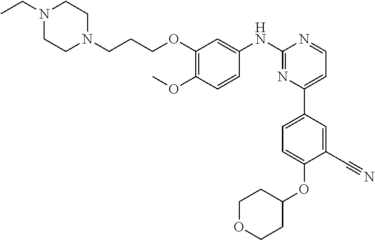 [SMILES: CCN1CCN(CCCOc2cc(Nc3nccc(-c4ccc(OC5CCOCC5)c(C#N)c4)n3)ccc2OC)CC1] | 5-[2-({3-[3-(4- ethylpiperazin-1- yl)propoxy]-4- methoxyphenyl} amino) pyrimidin-4-yl]-2- (tetrahydro-21H- pyran-4- yloxy)benzonitrile | ¹H NMR (DMSO-d₆) δ 9.57 (s, 1H), 9.55 (d, 1H), 8.52 (d, 1H), 8.43 (dd, 1H), 7.63 (br s, 1H), 7.55 (d, 1H), 7.44 (d, 1H), 7.25 (dd, 1H), 6.95 (d, 1H), 4.96 (sept., 1H), 4.09 (t, 2H), 3.92-3.84 (m, 2H), 3.76 (s, 3H), 3.56 (ddd, 2H), 3.80-3.50 (m, 4H), 3.36-3.00 (m, 8H), 2.24-2.12 (m, 2H), 2.10-2.00 (m, 2H), 1.75- 1.62 (m, 2H), 1.23 (t, 3H). LC-MS [M + H]⁺ 573.3174. |
| 526 | 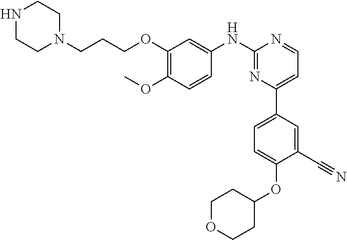 [SMILES: COc1ccc(Nc2nccc(-c3ccc(OC4CCOCC4)c(C#N)c3)n2)cc1OCCCN1CCNCC1] | 5-[2-({4-methoxy-3- [3-(piperazin-1- yl)propoxy]phenyl} amino)pyrimidin-4- yl]-2-(tetrahydro-2H- pyran-4- yloxy)benzonitrile | ¹H NMR (DMSO-d₆) δ 9.57 (s, 1H), 9.19 (br s, 2H), 8.55 (d, 1H), 8.52 (d, 1H), 8.43 (dd, 1H), 7.65 (br s, 1H), 7.55 (d, 1H), 7.44 (d, 1H), 7.25 (dd, 1H), 6.95 (d, 1H), 4.96 (sept., 1H), 4.10 (t, 1H), 3.93-3.82 (m, 2H), 3.75 (s, 3H), 3.57 (ddd, 2H), 3.62-3.10 (m, 10H), 2.24-2.12 (m, 2H), 2.10-2.00 (m, 2H), 1.74- 1.62 (m, 2H); LC-MS [M + H]⁺ 545.2859. |
| 527 | 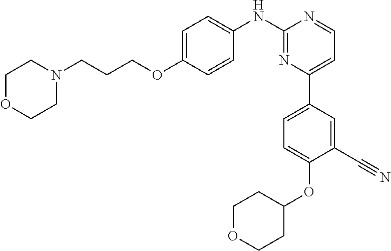 [SMILES: N#Cc1cc(-c2ccnc(Nc3ccc(OCCCN4CCOCC4)cc3)n2)ccc1OC1CCOCC1] | 5-[2-({4-[3- (morpholin-4- yl)propoxy]phenyl} amino)pyrimidin-4- yl]-2-(tetrahydro-2H- pyran-4- yloxy)benzonitrile | ¹H NMR (DMSO-d₆) δ 9.76 (br s, 1H), 9.56 (s, 1H), 8.52 (d, 1H), 8.51 (d, 1H), 8.43 (dd, 1H), 7.72- 7.66 (m, 2H), 7.54 (d, 1H), 7.42 (d, 1H), 6.96-6.90 (m, 2H), 4.95 (sept., 1H), 4.08-3.96 (m, 4H), 3.92-3.82 (m, 2H), 3.66 (t, 2H), 3.56 (ddd, 2H), 3.54-3.48 (m, 2H), 3.35-3.25 (m, 2H), 3.18-3.04 (m, 2H), 2.18- 2.08 (m, 2H), 2.08-1.98 (m, 2H), 1.75-1.63 (m, 2H); LC-MS [M + H]⁺ 516.260. |
| 528 | 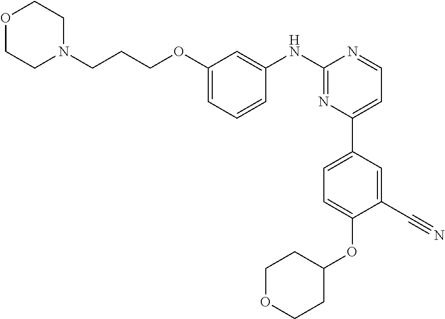 [SMILES: N#Cc1cc(-c2ccnc(Nc3cccc(OCCCN4CCOCC4)c3)n2)ccc1OC1CCOCC1] | 5-[2-({3-[3- (morpholin-4- yl)propoxy]phenyl} amino)pyrimidin-4- yl]-2-(tetrahydro-2H- pyran-4- yloxy)benzonitrile | ¹H NMR (DMSO-d₆) δ 9.78 (br s, 1H), 9.76 (s, 1H), 8.59-8.55 (m, 2H), 8.45 (dd, 1H), 7.66-7.62 (m, 1H), 7.56 (d, 1H), 7.50 (d, 1H), 7.33 (d, 1H), 7.23 (d, 1H), 7.58 (dd, 1H), 4.96 (sept., 1H), 4.09 (t, 2H), 4.05-3.91 (m, 2H), 3.92-3.83 (m, 2H), 3.66 (t, 2H), 3.56 (ddd, 2H), 3.55-3.46 (m, 2H), 3.37-3.26 (m, 2H), 3.18-3.02 (m, 2H), 2.23- 2.11 (m, 2H), 2.10-2.00 (m, 2H), 1.74-1.62 (m, 2H); LC-MS [M + H]⁺ 516.2590. |
| 529 | 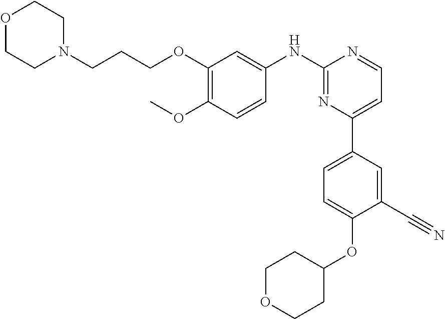 [SMILES: COc1ccc(Nc2nccc(-c3ccc(OC4CCOCC4)c(C#N)c3)n2)cc1OCCCN1CCOCC1] | 5-[2-({4-methoxy-3- [3-(morpholin-4- yl)propoxy]phenyl} amino)pyrimidin-4- yl]-2-(tetrahydro-2H- pyran-4- yloxy)benzonitrile | ¹H NMR (DMSO-d₆) δ 9.64-9.50 (m, 2H), 8.57-8.50 (m, 2H), 8.42 (d, 1H), 7.63 (s, 1H), 7.55 (d, 1H), 7.43 (d, 1H), 7.25 (d, 1H), 6.95 (d, 1H), 5.00-4.90 (m, 1H), 4.14-4.06 (m, 2H), 4.06-3.96 (m, 2H), 3.92- 3.82 (m, 2H), 3.76 (s, 3H), 3.70- 3.66 (t, 2H), 3.60-3.48 (m, 4H), 3.40-3.30 (m, 2H), 3.18-3.04 (m, 2H), 2.26-2.14 (m, 2H), 2.10-1.98 (m, 2H), 1.78-1.60 (m, 2H); LC- MS [M + H]⁺ 546.2732. |
| 530 | 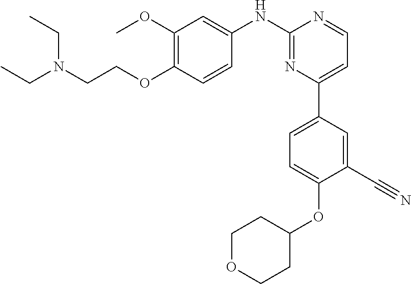 [SMILES: CCN(CC)CCOc1ccc(Nc2nccc(-c3ccc(OC4CCOCC4)c(C#N)c3)n2)cc1OC] | 5-[2-({4-[2- (diethylamino)ethoxy- 3- methoxyphenyl] amino) pyrimidin-4-yl}-2- (tetrahydro-2H- pyran-4- yloxy)benzonitrile | ¹H NMR (DMSO-d₆) δ 9.65 (s, 1H), 9.42 (s, 1H), 8.57 (d, 1H), 8.55 (d, 1H), 8.44 (dd, 1H), 7.77 (s, 1H), 7.55 (d, 1H), 7.46 (d, 1H), 7.23 (d, 1H), 7.02 (d, 1H), 4.96 (sept., 1H), 4.30-4.20 (m, 2H), 3.92-3.80 (m, 2H), 3.85 (s, 3H), 3.62-3.47 (m, 4H), 3.38-3.20 (m, 4H), 2.10-1.98 (m, 2H), 1.74-1.62 (m, 2H), 1.26 (t, 6H); LC-MS [M + H]⁺ 518.2773. |
| 531 | 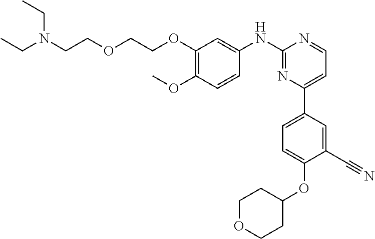 [SMILES: CCN(CC)CCOCCOc1cc(Nc2nccc(-c3ccc(OC4CCOCC4)c(C#N)c3)n2)ccc1OC] | 5-{2-[(3-{2-[2- (diethylamino)ethoxy] ethoxy}-4- methoxyphenyl) amino] pyrimidin-4-yl}-2- (tetrahydro-2H- pyran-4- yloxy)benzonitrile | ¹H NMR (DMSO-d₆) δ 9.54 (s, 1H), 9.11 (br s, 1H), 8.54 (d, 1H), 8.52 (d, 1H), 8.42 (dd, 1H), 7.62 (s, 1H), 7.54 (d, 1H), 7.43 (d, 1H), 7.25 (dd, 1H), 6.94 (d, 1H), 4.95 (sept., 1H), 4.20-4.12 (m, 2H), 3.93-3.78 (m, 6H), 3.75 (s, 3H), 3.56 (ddd, 2H), 3.31 (q, 2H), 3.25-3.09 (m, 4H), 2.10-1.98 (m, 2H), 1.75-1.62 (m, 2H), 1.17 (t, 6H); LC-MS [M + H]⁺ 562.3034 |
| 532 | 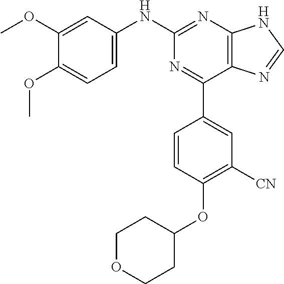 [SMILES: COc1ccc(Nc2nc(-c3ccc(OC4CCOCC4)c(C#N)c3)c3nc[nH]c3n2)cc1OC] | 5-{2-[(3,4- Dimethoxyphenyl) amino]-3H-purin-6-yl}- 2-(tetrahydro-2H- pyran-4- yloxy)benzonitrile | ¹H NMR (DMSO) δ 9.41-9.36 (m, 1H), 9.19-9.13 (m, 2H), 7.72-7.59 (m, 2H), 7.36-7.28 (m, 1H), 6.97- 6.90 (m, 1H), 5.01 (bs, 1H), 4.02- 4.05 (m, 2H), 3.92-3.60 (m, 8H), 2.10 (bs, 2H), 1.75 (bs, 2H). LC- MS [M + H]⁺ 473.1928. |
| 533 | 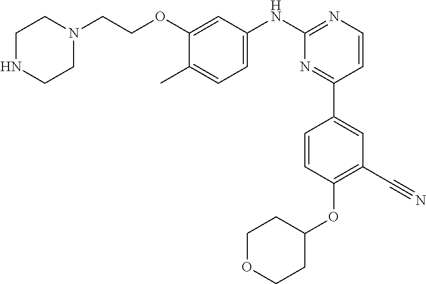 [SMILES: Cc1ccc(Nc2nccc(-c3ccc(OC4CCOCC4)c(C#N)c3)n2)cc1OCCN1CCNCC1] | 5-[2-({4-Methyl-3- [2-(piperazin-1- yl)ethoxy]phenyl} amino)pyrimidin- 4-yl]- 2-(tetrahydro-2H- pyran-4- yloxy)benzonitrile | ¹H NMR (DMSO-d₆) δ 9.66 (s, 1H), 8.97 (br. s., 2H), 8.51-8.58 (m, 2H), 8.44 (dd, 1H), 7.64 (s, 1H), 7.55 (d, 1H), 7.47 (d, 1H), 7.22-7.30 (m, 1H), 7.08 (d, 1H), 4.89-5.00 (m, 1H), 4.23-4.34 (m, 2H), 3.81-3.92 (m, 2H), 3.49- 3.61 (m, 4H), 3.42 (s, 2H), 3.32 (s, 6H), 2.15 (s, 3H), 1.96-2.09 (m, 2H), 1.64-1.75 (m, 2H); LC-MS [M + H]⁺ 515.2763 |
| 534 | 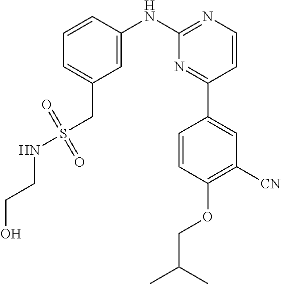 [SMILES: CC(C)COc1ccc(-c2ccnc(Nc3cccc(CS(=O)(=O)NCCO)c3)n2)cc1C#N] | 1-[3-({4-[3-Cyano-4- (2- methylpropoxy) phenyl] pyrimidin-2- yl}amino)phenyl]-N- (2- hydroxyethyl) methanesulfonamide | ¹H NMR (CDCl₃) δ 8.39 (d, 1H), 8.28-8.22 (m, 2H), 7.85 (s, 1H), 7.61 (d, 1H), 7.35-7.27 (m, 1H), 7.09-7.03 (m, 3H), 4.33 (s, 2H), 3.92 (d, 2H), 3.67-3.64 (m, 2H), 3.22-3.06 (m, 2H), 2.24-2.18 (m, 1H), 1.10 (d, 6H). LC-MS [M + H]⁺ 482.1871 |
| 535 | 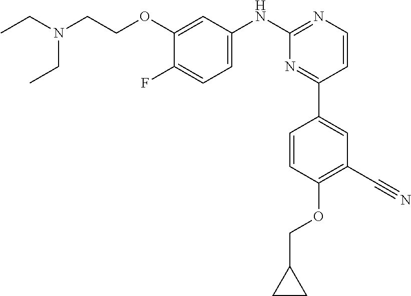 [SMILES: CCN(CC)CCOc1cc(Nc2nccc(-c3ccc(OCC4CC4)c(C#N)c3)n2)ccc1F] | 2- (Cyclopropylmethoxy)- 5-[2-({3-[2- (diethylamino)ethoxy]- 4- fluorophenyl}amino) pyrimidin-4- yl]benzonitrile | ¹H NMR (DMSO-d₆) δ ppm 9.79 (s, 1H), 9.56 (br. s., 1H), 8.51-8.59 (m, 2H), 8.45 (dd, 1H), 7.88 (d, 1H), 7.50 (d, 1H), 7.41 (d, 1H), 7.26-7.34 (m, 1H), 7.15-7.26 (m, 1H), 4.38-4.49 (m, 2H), 4.05- 4.16 (m, 2H), 3.63 (d, 2H), 3.22- 3.33 (m, 4H), 1.23-1.33 (m, 7H), 0.59-0.70 (m, 2H), 0.36-0.46 (m, 2H); LC-MS [M + H]⁺ 476.2459. |
| 536 | 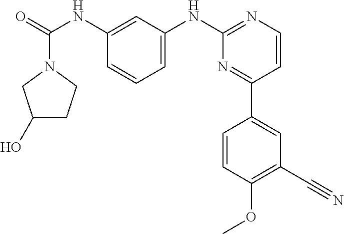 | N-[3-({4-[3-Cyano- 4-(tetrahydro-2H- pyran-4- yloxy)phenyl] pyrimidin-2- yl}amino)phenyl]-3- hydroxypyrrolidine- 1-carboxamide | ¹H NMR (MeOH-d₄) δ 8.49 (d, 1H), 8.44-8.41 (m, 2H), 8.11 (t, 1H), 7.33 (d, 1H), 7.28-7.19 (m, 3H), 7.05-7.03 (m, 1H), 4.98-4.90 (m, 1H), 4.04-3.96 (m, 2H), 3.68-3.60 (m, 5H), 3.47 (d, 1H), 2.15-2.07 (m, 3H), 2.03-1.96 (m, 1H), 1.88- 1.79 (m, 2H). LC-MS [M + H]⁺ 501.2234 |
| 537 | 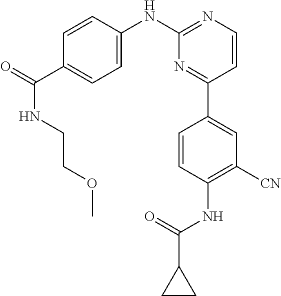 [SMILES: COCCNC(=O)c1ccc(Nc2nccc(-c3ccc(NC(=O)C4CC4)c(C#N)c3)n2)cc1] | 4-[(3-{3-Cyano-4- [(cyclopropylcarbonyl) amino]phenyl} pyrimidin-2-yl) amino]-N-(2- methoxyethyl) benzamide | ¹H NMR (CDCl₃) δ 8.52 (d, 1H), 8.38-8.36 (m, 2H), 8.27 (d, 1H), 7.85-7.82 (m, 4H), 7.26-7.19 (m, 2H), 3.77 (s, 3H), 3.63-3.61 (m, 2H), 3.47-3.38 (m, 2H), 1.85-1.81 (m, 1H), 1.16-1.14 (m, 2H), 1.00- 0.98 (m, 2H). LC-MS [M + H]⁺ 457.1938 |
| 538 | 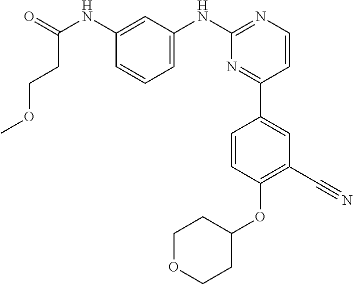 [SMILES: COCCC(=O)Nc1cccc(Nc2nccc(-c3ccc(OC4CCOCC4)c(C#N)c3)n2)c1] | N-[3-({4-[3-Cyano- 4-(tetrahydro-2H- pyran-4- yloxy)phenyl] pyrimidin-2- yl}amino)phenyl]-3- methoxypropanamide | ¹H NMR (DMSO-d₆) δ 9.93 (s, 1H), 9.69 (s, 1H), 8.62 (d, 1H), 8.56- 8.48 (m, 2H), 8.39 (s, 1H), 7.52- 7.46 (m, 2H), 7.32 (d, 1H), 7.24- 7.10 (m, 2H), 4.98-4.90 (m, 1H), 3.90-3.85 (m, 2H), 3.65 (t, 2H), 3.59-3.53 (m, 2H), 3.25 (s, 3H), 2.65 (t, 2H), 2.07-2.02 (m, 2H), 1.72-1.63 (m, 2H). LC-MS [M + H]⁺ 474.2124 |
| 539 | 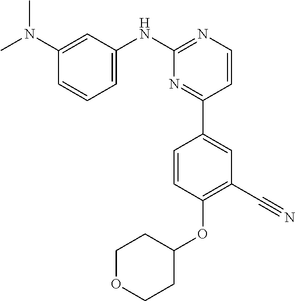 [SMILES: CN(C)c1cccc(Nc2nccc(-c3ccc(OC4CCOCC4)c(C#N)c3)n2)c1] | 5-(2-{[3- (Dimethylamino) phenyl]amino} pyrimidin- 4-yl)-2-(tetrahydro- 2H-pyran-4- yloxy)benzonitrile | ¹H NMR (CDCl₃) δ 8.60 (d, 1H), 8.39 (d, 1H), 8.23 (dd, 1H), 7.31- 7.30 (m, 1H), 7.24-7.20 (m, 2H), 7.05 (s, 1H), 7.05 (d, 1H), 6.89 (d, 1H), 6.48 (dd, 1H), 4.78-4.74 (m, 1H), 4.07-4.01 (m, 2H), 3.02 (s, 6H), 2.08-2.05 (m, 2H), 1.96-1.93 (m, 2H). LC-MS [M + H]⁺ 416.2090 |
| 540 | 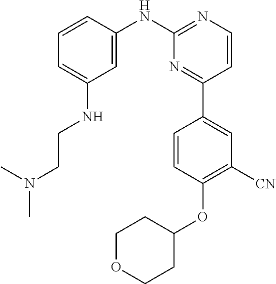 [SMILES: CN(C)CCNc1cccc(Nc2nccc(-c3ccc(OC4CCOCC4)c(C#N)c3)n2)c1] | 5-{2-[(3-{[2- (Dimethylamino) ethyl]amino}phenyl) amino]pyrimidin- 4-yl}-2- (tetrahydro-2H- pyran-4- yloxy)benzonitrile | ¹H NMR (CDCl₃) δ 8.46 (d, 1H), 8.44 (d, 1H), 8.25-8.22 (m, 1H), \7.19-7.13 (m, 3H), 7.08-7.03 (m, 2H), 6.92-6.90 (m, 1H), 6.39-6.37 (m, 1H), 4.76-4.73 (m, 1H), 4.39 (bs, 1H), 4.07-4.01 (m, 2H), 3.69- 3.63 (m, 2H), 3.28-3.18 (m, 2H), 2.61-2.58 (m, 2H), 2.26 (s, 6H), 2.12-2.05 (m, 2H), 1.97-1.88 (m, 2H). LC-MS [M + H]⁺ 459.2496 |
| 541 | 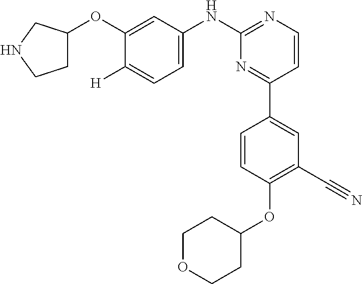 [SMILES: N#Cc1cc(-c2ccnc(Nc3cccc(OC4CCNC4)c3)n2)ccc1OC1CCOCC1] | 5-(2-{[4-Fluoro-3- (pyrrolidin-3- yloxy)phenyl]amino} pyrimidin-4-yl)-2- (tetrahydro-2H- pyran-4- yloxy)benzonitrile | ¹H NMR (DMSO-d₆) δ ppm 9.75 (s, 1H), 9.19 (br. s., 1H), 9.11 (br. s., 1H), 8.57 (d, 1H), 8.55 (d, 1H), 8.43 (dd, 1H), 7.73 (dd, 1H), 7.55 (d, 1H), 7.50 (d, 1H), 7.35-7.42 (m, 1H), 7.22 (dd, 1H), 5.13 (br. s., 1H), 4.91-4.99 (m, 1H), 3.84- 3.92 (m, 2H), 3.48-3.59 (m, 4H), 3.28-3.40 (m, 2H), 2.19-2.27 (m, 2H), 2.00-2.09 (m, 2H), 1.64- 1.74 (m, 2H); LC-MS [M + H]⁺ 476.2150. |
| 542 | 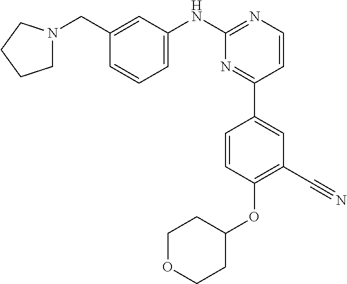 [SMILES: N#Cc1cc(-c2ccnc(Nc3cccc(CN4CCCC4)c3)n2)ccc1OC1CCOCC1] | 5-(2-{[3-(Pyrrolidin- 1- ylmethyl)phenyl] amino}pyrimidin- 4-yl)- 2-(tetrahydro-2H- pyran-4- yloxy)benzonitrile | ¹H NMR (DMSO-d₆) δ 9.94 (s, 1H), 9.83 (br s, 1H), 8.64-8.63 (m, 2H), 8.49 (dd, 1H), 8.00 (s, 1H), 7.83 (d, 1H), 7.61-7.56 (m, 2H), 7.45 (t, 1H), 7.18 (d, 1H), 5.03-4.98 (m, 1H), 4.41 (d, 2H), 3.95-3.90 (m, 2H), 3.67-3.57 (m, 4H), 3.20-3.15 (m, 2H), 2.16-2.05 (m, 4H), 1.94- 1.89 (m, 2H), 1.77-1.70 (m, 2H). LC-MS [M + H]⁺ 456.2428 |
| 543 | 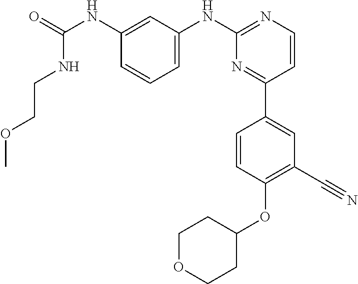 [SMILES: COCCNC(=O)Nc1cccc(Nc2nccc(-c3ccc(OC4CCOCC4)c(C#N)c3)n2)c1] | 1-[3-({4-[3-Cyano-4- (tetrahydro-2H- pyran-4- yloxy)phenyl]pyrimidin- 2- yl}amino)phenyl]-3- (2-methoxyethyl)urea | ¹H NMR (MeOH-d₄) δ 8.50 (d, 1H), 8.45-8.42 (m, 2H), 8.07-8.06 (m, 1H), 7.33 (d, 1H), 7.27-7.17 (m, 3H), 6.92 (d, 1H), 4.98-4.90 (m, 1H), 4.15-3.94 (m, 2H), 3.67-3.62 (m, 2H), 3.49 (t, 2H), 3.40 (t, 2H), 3.38 (s, 3H), 2.15-2.07 (m, 2H), 1.88-1.79 (m, 2H). LC-MS [M + H]⁺ 489.2231 |
| 544 | 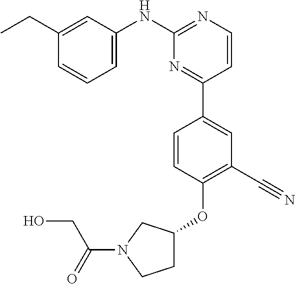 [SMILES: CCc1cccc(Nc2nccc(-c3ccc(O[C@@H]4CCN(C(=O)CO)C4)c(C#N)c3)n2)c1] | 5-{2-[(3- Ethylphenyl)amino] pyrimidin-4-yl}-2- {[(3R)-1- (hydroxyacetyl) pyrrolidin-3- yl]oxy}benzonitrile | ¹H NMR (DMSO-d6) δ 9.65 (s, 1H), 8.57-8.55 (m, 2H), 8.49-8.46 (m, 1H), 7.76 (s, 1H), 7.56-7.47 (m, 3H), 7.22 (t, 1H), 6.84 (d, 1H), 5.43-5.34 (m, 1H), 4.11-3.83 (m, 2H), 3.69-3.58 (m, 3H), 3.51-3.36 (m, 2H), 2.65-2.58 (m, 2H), 2.32- 2.12 (m, 2H), 1.23 (t, 3H). LC-MS [M + H]⁺ 444.2065 |
| 545 | 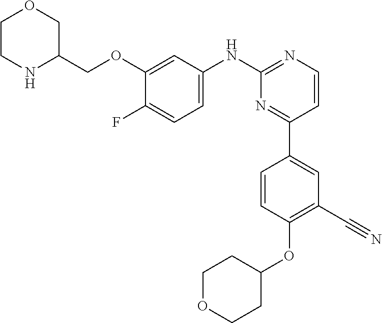 [SMILES: N#Cc1cc(-c2ccnc(Nc3ccc(F)c(OCC4COCCN4)c3)n2)ccc1OC1CCOCC1] | 5-(2-{[4-Fluoro-3- (morpholin-3- ylmethoxy)phenyl] amino}pyrimidin-4- yl)-2-(tetrahydro-2H- pyran-4- yloxy)benzonitrile] | ¹H NMR (DMSO-d₆) δ ppm 9.77 (s, 1H), 9.37 (br. s., 1H), 9.14 (br. s., 1H), 8.54-8.58 (m, 2H), 8.45 (dd, 1H), 7.81 (dd, 1H), 7.55 (d, 1H), 7.51 (d, 1H), 7.33-7.42 (m, 1H), 7.23 (dd, 1H), 4.89-4.99 (m, 1H), 4.20-4.31 (m, 2H), 4.09 (dd, 1H), 3.92-3.99 (m, 1H), 3.84-3.91 (m, 2H), 3.80 (s, 1H), 3.62-3.73 (m, \2H), 3.56 (ddd, 2H), 3.28-3.34 (m, 1H), 3.18-3.26 (m, 1H), 1.99- 2.09 (m, 2H), 1.64-1.75 (m, 2H); LC-MS [M + H]⁺ 506.2163. |
| 546 | 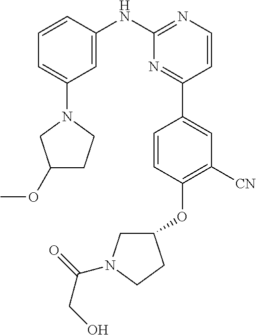 [SMILES: COC1CCN(c2cccc(Nc3nccc(-c4ccc(O[C@@H]5CCN(C(=O)CO)C5)c(C#N)c4)n3)c2)C1] | 2-{[(3R)-1- (Hydroxyacetyl) pyrrolidin-3-yl] oxy}-5-(2-{[3-(3- methoxypyrrolidin-1- yl)phenyl]amino} pyrimidin-4- yl)benzonitrile | ¹H NMR (MeOH-d₄) δ 8.57-8.39 (m, 2H), 7.88 (s, 1H), 7.38-7.35 (m, 2H), 7.28-7.24 (m, 1H), 7.15-7.12 (m, 1H), 6.86-6.83 (m, 1H), 6.35- 6.26 (m, 1H), 5.39-5.33 (m, 1H), 4.21 (d, 2H), 3.85-3.49 (m, 6-H), 3.38 (s, 3H), 2.43-2.18 (m, 4H), 1.66-1.59 (m, 2H). LC-MS [M + H]⁺ 515.2402 |
| 547 | 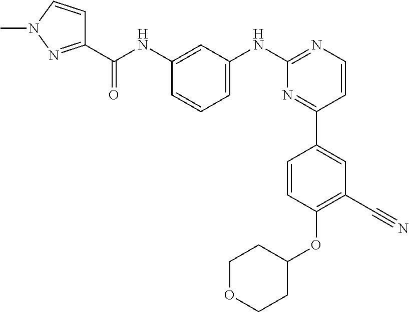 [SMILES: Cn1ccc(C(=O)Nc2cccc(Nc3nccc(-c4ccc(OC5CCOCC5)c(C#N)c4)n3)c2)n1] | N-[3-({4-[3-Cyano- 4-(tetrahydro-2H- pyran-4- yloxy)phenyl] pyrimidin-2- yl}amino)phenyl]-1- methyl-1H-pyrazole- 3-carboxamide | ¹H NMR (CDCl₃) δ 8.82-8.78 (m, 2H), 8.58 (s, 1H), 8.49 (dd, 1H), 8.19 (d, 2H), 7.43 (s, 1H), 7.34 (t, 1H), 7.27-7.25 (m, 1H), 7.21 (d, 1H), 7.17-7.13 (m, 2H), 6.95 (s, 1H) 4.82-4.76 (m, 1H), 4.06-3.99 (m, 5H), 3.69-3.63 (m, 2H), 2.13- 2.05 (m, 2H), 1.97-1.89 (m, 2H). LC-MS [M + H]⁺ 496.2154 |
| 548 | 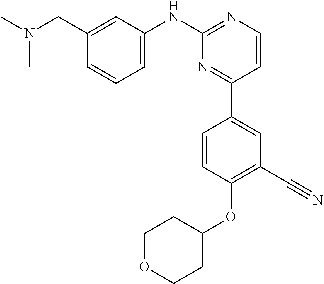 [SMILES: CN(C)Cc1cccc(Nc2nccc(-c3ccc(OC4CCOCC4)c(C#N)c3)n2)c1] | 5-[2-({3- [(Dimethylamino) methyl]phenyl} amino) pyrimidin-4-yl]-2- (tetrahydro-2H- pyran-4- yloxy)benzonitrile | ¹H NMR (CDCl₃) δ 9.90 (s, 1H), 8.59-8.58 (m, 2H), 8.44 (d, 1H), 7.98 (s, 1H), 7.79 (d, 1H), 7.56- 7.52 (m, 2H), 7.43 (t, 1H), 7.12 (m, 1H), 5.00-4.92 (m, 1H), 4.28 (s, 2H), 3.90-3.86 (m, 2H), 3.59-3.54 (m, 2H), 2.78 (s, 6H), 2.10-2.01 (m, 2H), 1.74-1.66 (m, 2H). LC-MS [M + H]⁺ 430.2248 |
| 549 | 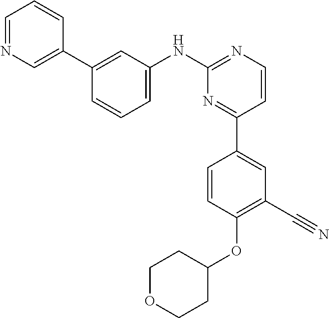 [SMILES: N#Cc1cc(-c2ccnc(Nc3cccc(-c4cccnc4)c3)n2)ccc1OC1CCOCC1] | 5-(2-{[3-(Pyridin-3- yl)phenyl]amino} pyrimidin-4-yl)-2- (tetrahydro-2H- pyran-4- yloxy)benzonitrile | ¹H NMR (CDCl₃) δ 9.89 (s, 1H), 9.01 (m, 1H), 8.72 (m, 1H), 8.57 (m, 2H), 8.46 (m, 1H), 8.35-7.30 (m, 2H), 7.86 (m, 1H), 7.77-7.74 (m, 1H), 7.57-7.47 (m, 2H), 7.38 (m, 1H), 4.96 (m, 1H), 3.91-3.85 (m, 2H), 3.59-3.54 (m, 2H), 2.07- 2.03 (m, 2H), 1.74-1.65 (m, 2H). LC-MS [M + H]⁺ 450.1964 |
| 550 | 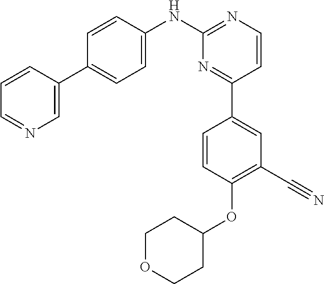 [SMILES: N#Cc1cc(-c2ccnc(Nc3ccc(-c4cccnc4)cc3)n2)ccc1OC1CCOCC1] | 5-(2-{[4-(Pyridin-3- yl)phenyl]amino} pyrimidin-4-yl)-2- (tetrahydro-2H- pyran-4- yloxy)benzonitrile | ¹H NMR (CDCl₃) δ 10.03 (s, 1H), 9.11 (s, 1H), 8.78-8.52 (m, 5H), 8.05 (m, 2H), 7.85 (m, 2H), 7.63- 7.59 (m, 2H), 5.01 (m, 1H), 3.93 (m, 2H), 3.63-3.59 (m, 2H), 2.09 (m, 2H), 1.75 (m, 2H). LC-MS [M + H]⁺ 450.1879 |
| 551 | 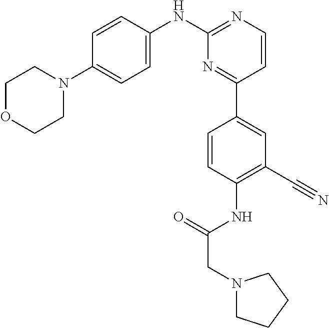 [SMILES: N#Cc1cc(-c2ccnc(Nc3ccc(N4CCOCC4)cc3)n2)ccc1NC(=O)CN1CCCC1] | N-[2-Cyano-4-(2- {[4-(morpholin-4- yl)phenyl]amino} pyrimidin-4-yl) phenyl]- 2-(pyrrolidin-1- yl)acetamide | ¹H NMR (DMSO-d6) δ 8.40 (d, 1H), 8.33 (d, 1H), 8.26 (d, 1H), 7.62 (d, 2H), 7.29 (d, 1H), 6.93- 6.88 (m, 3H), 4.10-4.07 (m, 2H), 3.76-3.73 (m, 4H), 3.06-3.03 (m, 4H), 1.95-1.91 (m, 2H), 1.85-1.79 (m, 2H), 1.27-1.20 (m, 4H). LC- MS [M + H]⁺ 484.2433 |
| 552 | 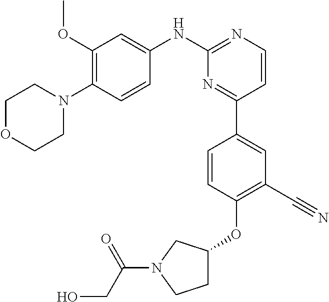 | 5-(5-Fluoro-2-{[3- methoxy-4- (morpholin-4- yl)phenyl]amino} pyrimidin-4-yl)-2- {[(3R)-1- (hydroxyacetyl) pyrrolidin-3- yl]oxy}benzonitrile | ¹H NMR (DMSO-d₆) δ ppm 9.67 (s, 1H), 8.63 (d, 1H), 8.40 (d, 1H), 8.31-8.37 (m, 1H), 7.54-7.62 (m, 2H), 7.18 (dd, 1H), 6.84 (d, 1H), 5.29-5.46 (m, 1H), 4.65-4.72 (m, 1H), 4.05-4.08 (m, 1H), 3.97- 4.04 (m, 1H), 3.81 (s, 3H), 3.69- 3.74 (m, 5H), 3.59-3.69 (m, 2H), 3.41-3.53 (m, 1H), 2.87-2.93 (m, 4H), 2.22-2.34 (m, 1H), 2.10- 2.22 (m, 1H); LC-MS [M + H]⁺ 549.2289 |
| 553 | 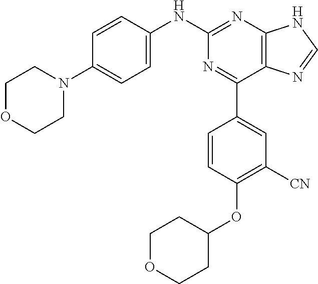 [SMILES: N#Cc1cc(-c2nc(Nc3ccc(N4CCOCC4)cc3)nc3[nH]cnc23)ccc1OC1CCOCC1] | 5-(2-{[4-(Morpholin- 4-yl)phenyl]amino}- 3H-purin-6-yl)-2- (tetrahydro-2H- pyran-4- yloxy)benzonitrile] | ¹H NMR (DMSO) δ 9.42 (bs, 1H), 9.13-9.03 (m, 2H), 8.31 (bs, 1H), 7.75-7.60 (m, 3H), 7.06 (bs, 2H), 4.96 (bs, 1H), 3.91-3.80 (m, 6H), 3.59-3.55 (m, 2H), 3.24 (bs, 4H), 2.08-2.06 (m, 2H), 1.72-1.70 (m, 2H). LC-MS [M + H]⁺ 498.2181. |
| 554 | 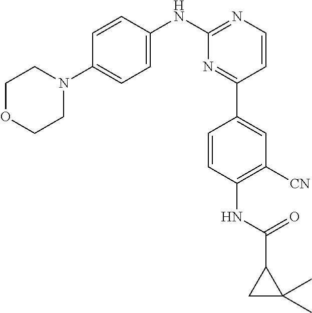 [SMILES: CC1(C)CC1C(=O)Nc1ccc(-c2ccnc(Nc3ccc(N4CCOCC4)cc3)n2)cc1C#N] | N-[2-Cyano-4-(2- {[4-(morpholin-4- yl)phenyl]amino} pyrimidin-4-yl)phenyl]- 2,2- dimethylcyclopropane- carboxamide | ¹H NMR (CDCl₃) δ 8.50 (d, 1H), 8.44 (d, 1H), 8.31 (s, 1H), 8.20 (d, 1H), 7.95 (s, 1H), 7.54 (d, 2H), 7.32 (s, 1H), 7.03 (d, 1H), 6.95 (d, 2H), 3.89-3.87 (m, 4H), 3.16-3.13 (m, 4H), 1.56-1.54 (m, 1H), 1.31- 1.25 (m, 7H), 0.99-0.96 (m, 1H). LC-MS [M + H]⁺ 469.2343 |
| 555 | 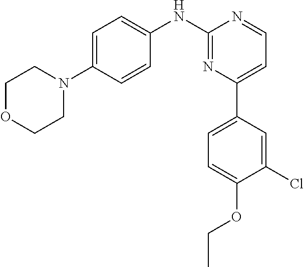 [SMILES: CCOc1ccc(-c2ccnc(Nc3ccc(N4CCOCC4)cc3)n2)cc1Cl] | 4-(3-Chloro-4- ethoxyphenyl)-N-[4- (morpholin-4- yl)phenyl]pyrimidin- 2-amine | LC-MS [M + H]⁺ 411.1632 |
| 556 | 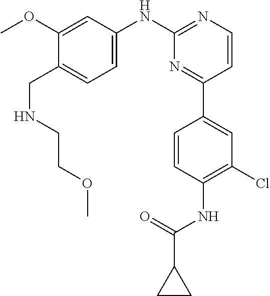 | N-(2-Cyano-4-{2- [(3-methoxy-4-{[(2- methoxyethyl)amino] methyl}phenyl) amino]pyrimidin-4- yl}phenyl) cyclopropane- carboxamide] | ¹H NMR (CDCl₃) δ 8.48 (d, 1H), 8.36-8.34 (m, 2H), 8.20-8.18 (m, 1H), 7.83 (d, 1H), 7.30 (d, 1H), 7.16 (d, 1H), 7.03 (d, 1H), 4.16 (s, 2H), 3.94 (s, 3H), 3.72-3.69 (m, 2H), 3..40 (s, 3H), 3.13-3.11 (m, 2H), 1.87-1.80 (m, 1H), 1.18-1.16 (m, 2H), 1.01-0.97 (m, 2H). LC- MS [M + H]⁺ 473.2308 |
| 557 | 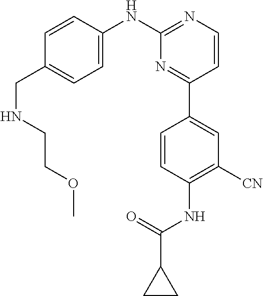 [SMILES: COCCNCc1ccc(Nc2nccc(-c3ccc(NC(=O)C4CC4)c(C#N)c3)n2)cc1] | N-(2-Cyano-4-{2- [(4-{[(2- methoxyethyl)amino] methyl}phenyl) amino]pyrimidin-4- yl}phenyl) cyclopropane- carboxamide] | ¹H NMR (CDCl₃) δ 8.47 (d, 1H), 8.42 (d, 1H), 8.31 (d, 1H), 8.23- 8.21 (m, 1H), 7.74 (d, 2H), 7.48 (d, 2H), 7.14 (d, 1H), 4.13 (s, 2H), 3.74-3.71 (m, 2H), 3.40 (s, 3H), 3.10-3.07 (m, 2H), 1.82-1.77 (m, 1H), 1.18-1.14 (m, 2H), 1.01-0.98 (m, 2H). LC-MS [M + H]⁺ 443.2186 |
| 558 | 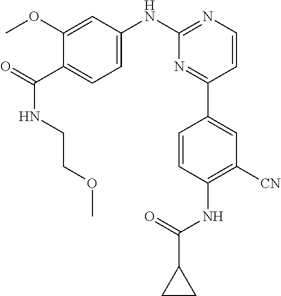 [SMILES: COCCNC(=O)c1ccc(Nc2nccc(-c3ccc(NC(=O)C4CC4)c(C#N)c3)n2)cc1OC] | 4-[(4-{3-Cyano-4- [(cyclopropylcarbonyl) amino]phenyl} pyrimidin-2-yl) amino]-2- methoxy-N-(2- methoxyethyl) benzamide | ¹H NMR (CDCl₃) δ 8.53-8.51 (m, 2H), 8.38 (d, 1H), 8.27-8.20 (m, 2H), 8.14 (d, 1H), 7.97 (d, 1H), 7.17 (d, 1H), 7.01 (dd, 1H), 4.05 (s, 3H), 3.69-3.58 (m, 4H), 3.43 (s, 3H), 1.78-1.73 (m, 1H), 1.19-1.15 (m, 2H), 1.02-0.99 (m, 2H). LC- MS [M + H]⁺ 487.2088 |
| 559 | 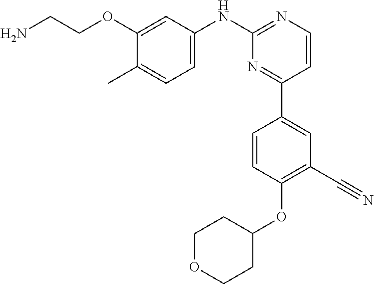 [SMILES: Cc1ccc(Nc2nccc(-c3ccc(OC4CCOCC4)c(C#N)c3)n2)cc1OCCN] | 5-(2-{[3-(2- Aminoethoxy)-4- methylphenyl]amino} pyrimidin-4-yl)-2- (tetrahydro-2H- pyran-4- yloxy)benzonitrile | ¹H NMR (DMSO-d₆) δ ppm 9.67 (s, 1H), 8.53-8.61 (m, 2H), 8.44 (dd, 1H), 7.98 (s, 3H), 7.64 (s, 1H), 7.56 (d, 1H), 7.47 (d, 1H), 7.26 (dd, 1H), 7.08 (d, 1H), 4.92-4.99 (m, 1H), 4.20 (t, 2H), 3.84-3.91 (m, 1H), 3.52-3.60 (m, 2H), 3.28- 3.38 (m, 2H), 2.19 (s, 3H), 2.01- 2.08 (m, 2H), 1.65-1.74 (m, 2H); LC-MS [M + H]⁺ 446.2198. |
| 560 | 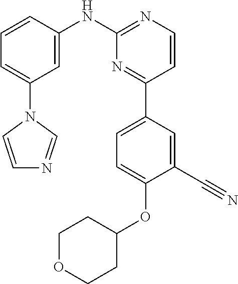 [SMILES: N#Cc1cc(-c2ccnc(Nc3cccc(-n4ccnc4)c3)n2)ccc1OC1CCOCC1] | 5-(2-{[3-(1H- Imidazol-1- yl)phenyl]amino} pyrimidin-4-yl)-2- (tetrahydro-2H- pyran-4- yloxy)benzonitrile | ¹H NMR (CDCl₃) δ 10.13 (s, 1H), 9.38 (s, 1H), 8.63 (m, 1H), 8.56 (m, 1H), 8.47-8.42 (m, 2H), 8.18 (s, 1H), 7.81-7.78 (m, 2H), 7.58-7.52 (m, 2H), 7.35-7.33 (m, 2H), 4.96 (m, 1H), 3.90-3.85 (m, 2H), 3.59- 3.54 (m, 2H), 2.07-2.02 (m, 2H), 1.73-1.66 (m, 2H). LC-MS [M + H]⁺ 439.1917 |
| 561 | 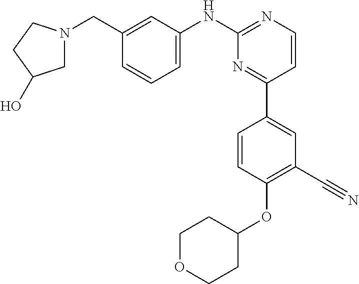 [SMILES: N#Cc1cc(-c2ccnc(Nc3cccc(CN4CCC(O)C4)c3)n2)ccc1OC1CCOCC1] | 5-[2-({3-[(3- Hydroxypyrrolidin- 1- yl)methyl]phenyl} amino)pyrimidin-4-yl]- 2-(tetrahydro-2H- pyran-4- yloxy)benzonitrile | ¹H NMR (DMSO-d₆) δ 9.98 (s, 1H), 8.60-8.58 (m, 2H), 8.46-8.44 (m, 2H), 8.20 (d, 1H), 7.79 (t, 1H), 7.56-7.52 (m, 2H), 7.16 (m, 1H), 5.00-4.90 (m, 1H), 4.48-4.30 (m, 3H), 3.90-3.85 (m, 2H), 3.60-3.53 (m, 3H), 3.27-3.10 (m, 2H), 2.06- 2.00 (m, 2H), 1.76-1.769 (m, 2H). LC-MS [M + H]⁺ 472.2337 |
| 562 | 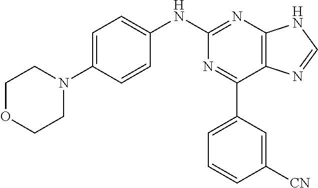 [SMILES: N#Cc1cccc(-c2nc(Nc3ccc(N4CCOCC4)cc3)nc3[nH]cnc23)c1] | 3-(2-{[4-(Morpholin- 4-yl)phenyl]amino}- 3H-purain-6- yl)benzonitrile | ¹H NMR (DMSO) δ 9.51 (bs, 1H), 9.18 (bs, 1H), 9.08 (m, 1H), 8.37 (bs, 1H), 8.05 (dt, 1H), 7.86 (t, 1H), 7.81-7.76 (m, 2H), 7.11 (bs, 1H), 3.81 (bs, 4H), 3.19 (bs, 4H). LC-MS [M + H]⁺ 398.1749. |
| 563 | 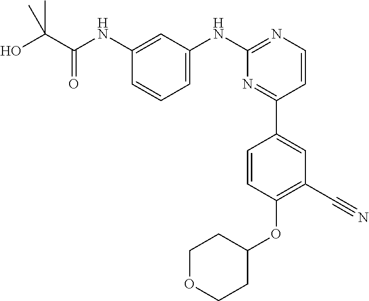 [SMILES: CC(C)(O)C(=O)Nc1cccc(Nc2nccc(-c3ccc(OC4CCOCC4)c(C#N)c3)n2)c1] | N-[3-({4-[3-Cyano- 4-(tetrahydro-2H- pyran-4- yloxy)phentyl] pyrimidin-2- yl}amino)phenyl]-2- hydroxy-2- methylpropanamide | ¹H NMR (CDCl₃) δ 8.86 (s, 1H), 8.56-8.54 (m, 2H), 8.39 (dd, 1H), 8.23 (d, 1H), 7.34 (t, 1H), 7.28- 7.24 (m, 2H), 7.21 (t, 1H), 4.82- 4.75 (m, 1H), 4.06-4.00 (m, 2H), 3.70-3.65 (m, 2H), 2.13-2.06 (m, 2H), 1.96-1.77 (m, 2H), 1.60 (s, 6H). LC-MS [M + H]⁺ 474.2109 |
| 564 | 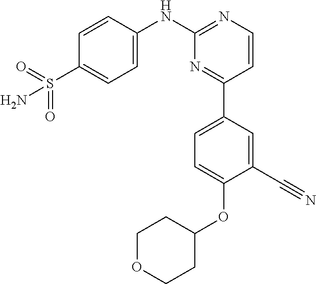 [SMILES: N#Cc1cc(-c2ccnc(Nc3ccc(S(N)(=O)=O)cc3)n2)ccc1OC1CCOCC1] | 4-({4-[3-Cyano-4- (tetrahydro-2H- pyran-4- yloxy)phenyl] pyrimidin-2- yl}amino) benzenesulfonamide | ¹H NMR (CDCl₃) δ 10.14 (s, 1H), 8.63 (d, 1H), 8.58 (d, 1H), 8.50- 8.47 (m, 1H), 7.99-7.96 (m, 2H), 7.78-7.75 (m, 2H), 7.60-7.56 (m, 2H), 7.22 (d, 2H), 4.95 (m, 1H), 3.91-3.85 (m, 2H), 3.59-3.53 (m, 2H), 2.07-2.03 (m, 2H), 1.74-1.66 (m, 2H). LC-MS [M + H]⁺ 452.1386 |
| 565 | 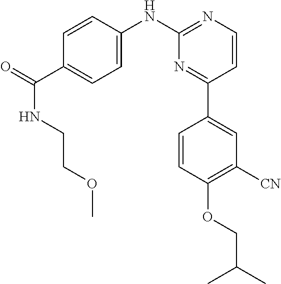 [SMILES: COCCNC(=O)c1ccc(Nc2nccc(-c3ccc(OCC(C)C)c(C#N)c3)n2)cc1] | 4-({4-[3-Cyano-4-(2- methylpropoxy)phenyl] pyrimidin-2- yl}amino)-N-(2- methoxyethyl) benzamide | ¹H NMR (CDCl₃) δ 8.49 (d, 1H), 8.30-8.27 (m, 2H), 7.84-7.78 (m, 4H), 7.16-7.0 (m, 2H), 3.94 (d, 2H), 3.66-3.59 (m, 4H), 3.42 (s, 3H), 2.24-2.19 (m, 1H), 1.11 (d, 6H). LC-MS [M + H]⁺ 446.2186 |
| 566 | 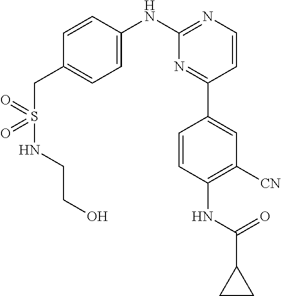 [SMILES: N#Cc1cc(-c2ccnc(Nc3ccc(CS(=O)(=O)NCCO)cc3)n2)ccc1NC(=O)C1CC1] | N-(2-Cyano-4-{2- [(4-{[(2- hydroxyethyl)sulfamoyl] methyl}phenyl)amino] pyrimidin-4- yl}phenyl) cyclopropane- carboxamide | ¹H NMR (CDCl₃) δ 8.44 (d, 1H), 8.39 (s, 1H), 8.28-8.23 (m, 2H), 7.74 (d, 2H), 7.41 (d, 2H), 7.12- 7.08 (m, 2H), 4.30 (s, 2H), 3.62- 3.59 (m, 2H), 3.13-3.10 (m, 2H), 1.86-1.83 (m, 1H), 1.14-1.09 (m, 2H), 1.00-0.97 (m, 2H). LC-MS [M + H]⁺ 493.1648 |
| 567 | 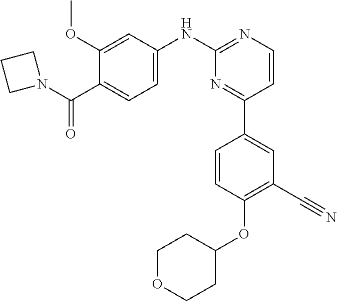 [SMILES: COc1cc(Nc2nccc(-c3ccc(OC4CCOCC4)c(C#N)c3)n2)ccc1C(=O)N1CCC1] | 5-(2-{[4-(Azetidin-1- ylcarbonyl)-3- methoxyphenyl]amino} pyrimidin-4-yl)-2- (tetrahydro-2H- pyran-4- yloxy)benzonitrile | LC-MS [M + H]⁺ 486.2142 |
| 568 | 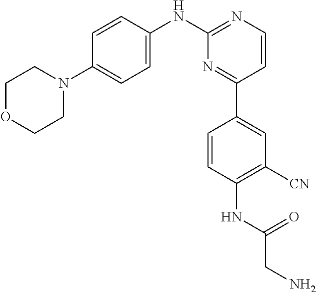 [SMILES: N#Cc1cc(-c2ccnc(Nc3ccc(N4CCOCC4)cc3)n2)ccc1NC(=O)CN] | N-[2-Cyano-4-(2- {[4-(morpholin-4- yl)phenyl]amino} pyrimidin-4- yl)phenyl] glycinamide | ¹H NMR (CDCl₃) δ 8.52 (s, 1H), 8.47-8.42 (m, 2H), 8.28 (d, 1H), 7.63 (d, 2H), 7.30 (d, 1H), 6.98 (d, 2H), 3.84-3.82 (m, 4H), 3.65 (s, 2H), 3.16-3.13 (m, 4H). LC-MS [M + H]⁺ 430.1960 |
| 569 | 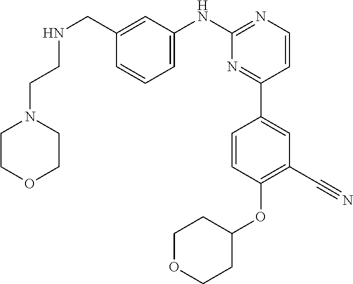 [SMILES: N#Cc1cc(-c2ccnc(Nc3cccc(CNCCN4CCOCC4)c3)n2)ccc1OC1CCOCC1] | 5-(2-{[3-({[2- (Morpholin-4- yl)ethyl]amino} methyl)phenyl]amino} pyrimidin-4-yl)-2- (tetrahydro-2H- pyran-4- yloxy)benzonitrile | LC-MS [M + H]⁺ 515.2768 |
| 570 | 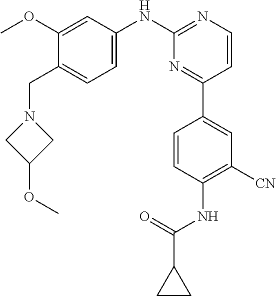 [SMILES: COc1cc(Nc2nccc(-c3ccc(NC(=O)C4CC4)c(C#N)c3)n2)ccc1CN1CC(OC)C1] | N-{2-Cyano-4-[2- ({3-methoxy-4-[(3- methoxyazetidin-1- yl)methyl]phenyl} amino)pyrimidin-4- yl]phenyl} cyclopropane- carboxamide | ¹H NMR (CDCl₃) δ 8.53-8.49 (m, 2H), 8.40 (d, 1H), 8.25-8.22 (m, 1H), 7.79 (s, 1H), 7.35-7.29 (m, 1H), 7.16 (d, 1H), 7.08 (d, 1H), 4.28-4.13 (m, 3H), 4.09 (s, 2H), 3.97 (s, 3H), 3.54-3.49 (m, 2H), 3.29 (s, 3H), 1.79-1.74 (m, 1H), 1.18-1.16 (m, 2H), 1.01-0.98 (m, 2H). LC-MS [M + H]⁺ 485.2306 |
| 571 | 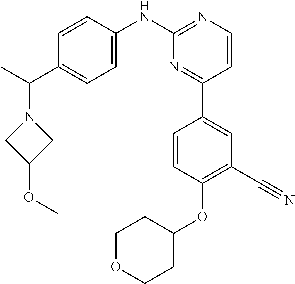 [SMILES: COC1CN(C(C)c2ccc(Nc3nccc(-c4ccc(OC5CCOCC5)c(C#N)c4)n3)cc2)C1] | 5-[2-({4-[1-(3- Methoxyazetidin-1- yl)ethyl]phenyl} amino)pyrimidin-4-yl]-2- (tetrahydro-2H- pyran-4- yloxy)benzonitrile | ¹H NMR (DMSO-d₆) δ 9.65 (s, 1H), 8.55-8.53 (m, 2H), 8.45 (dd, 1H), 7.71 (s, 2H), 7.56 (d, 1H), 7.46 (d, 1H), 7.22 (d, 2H), 4.98-4.92 (m, 1H), 3.92-3.85 (m, 3H), 3.59-3.53 (m, 3H), 3.24-3.17 (m, 2H), 3.13 (s, 3H), 2.79 (t, 1H), 2.65 (t, 1H), 2.09-2.02 (m, 2H), 1.73-1.65 (m, 2H), 1.12 (d, 3H). LC-MS [M + H]⁺ 486.2543 |
| 572 | 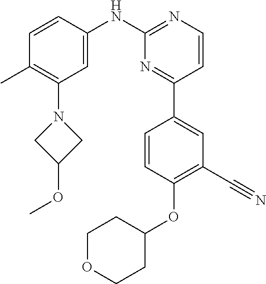 [SMILES: COC1CN(c2cc(Nc3nccc(-c4ccc(OC5CCOCC5)c(C#N)c4)n3)ccc2C)C1] | 5-(2-{[3-(3- Methoxyazetidin-1- yl)-4- methylphenyl]amino} pyrimidin-4-yl)-2- (tetrahydro-2H- pyran-4- yloxy)benzonitrile | ¹H NMR (DMSO-d₆) δ 9.52 (s, 1H), 8.56-8.53 (m, 2H), 8.46 (dd, 1H), 7.56 (d, 1H), 7.45 (d, 1H), 7.19 (s, 1H), 7.11 (d, 1H), 6.94 (d, 1H), 4.99-4.92 (m, 1H), 4.30-4.25 (m, 1H), 4.16 (t, 2H), 3.92-3.86 (m, 2H), 3.68-3.65 (m, 2H), 3.61-3.54 (m, 2H), 3.27 (s, 3H), 2.13 (s, 3H), 2.09-1.99 (m, 2H), 1.75-1.67 (m, 2H). LC-MS [M + H]⁺ 472.2352 |
| 573 | 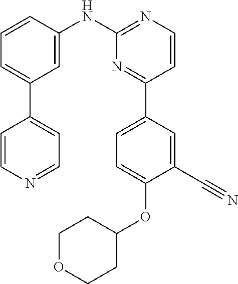 [SMILES: N#Cc1cc(-c2ccnc(Nc3cccc(-c4ccncc4)c3)n2)ccc1OC1CCOCC1] | 5-(2-{[3-(Pyridin-4- yl)phenyl]amino} pyrimidin-4-yl)-2- (tetrahydro-2H- pyran-4- yloxy)benzonitrile | ¹H NMR (CDCl₃) δ 9.9 (s, 1H),8.68 (m, 2H), 8.61 (m, 2H), 8.47 (m, 1H), 8.36 (m, 1H), 7.85 (m, 1H), 7.69 (m, 2H), 7.56-7.39 (m, 4H), 4.97 (m, 1H), 3.91-3.85 (m, 2H), 3.60-3.50 (m, 2H), 2.07-2.04 (m, 2H), 1.74-1.67 (m, 2H). LC- MS [M + H]⁺ 450.1860 |
| 574 | 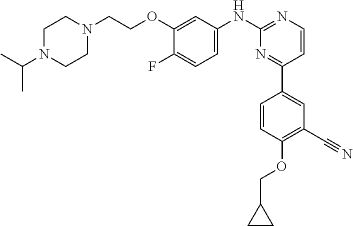 [SMILES: CC(C)N1CCN(CCOc2cc(Nc3nccc(-c4ccc(OCC5CC5)c(C#N)c4)n3)ccc2F)CC1] | 2- (Cyclopropylmethoxy)- 5-{2-[(4-fluoro-3- {2-[4-(propan-2- yl)piperazin-1- yl]ethoxy}phenyl) amino]pyrimidin-4- yl}benzonitrile | ¹H NMR (DMSO-d₆) δ ppm 9.76 (s, 1H), 8.53-8.62 (m, 2H), 8.45 (dd, 1H), 7.86 (d, 1H), 7.49 (d, 1H), 7.41 (d, 1H), 7.23-7.34 (m, 1H), 7.13-7.23 (m, 1H), 4.30 (br. s., 2H), 4.12 (d, 2H), 3.41-3.53 (m, 4H), 3.36 (br. s., 2H), 3.18 (br. s., 2H), 3.09 (br. s., 2H), 2.79 (br. s., 2H), 1.26 (d, 6H), 0.58-0.69 (m, 2H), 0.36-0.46 (m, 2H); LC-MS [M + H]⁺ 531.2867. |
| 575 | 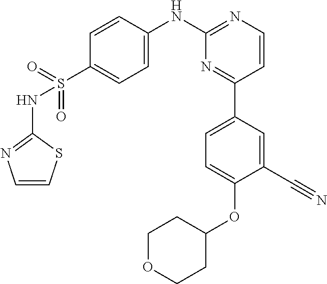 [SMILES: N#Cc1cc(-c2ccnc(Nc3ccc(S(=O)(=O)Nc4nccs4)cc3)n2)ccc1OC1CCOCC1] | 4-({4-[3-Cyano-4- (tetrahydro-2H- pyran-4- yloxy)phenyl] pyrimidin-2-yl} amino)-N- (1,3-thiazol-2- yl) benzenesulfonamide | ¹H NMR (CDCl₃) δ 12.66 (s, 1H), 10.14 (s, 1H), 8.62 (d, 1H), 8.57 (d, 1H), 8.48 (m, 1H), 7.97-7.94 (m, 2H), 7.76-7.74 (m, 2H), 7.59-7.56 (m, 2H), 7.26-7.24 (m, 1H), 6.82 (d, 1H), 6.57 (s, 1H), 4.95 (m, 1H), 3.91-3.85 (m, 2H), 3.59-3.53 (m, 2H), 2.07-2.03 (m, 2H), 1.74-1.65 (m, 2H). LC-MS [M + H]⁺ 535.1288 |
| 576 | 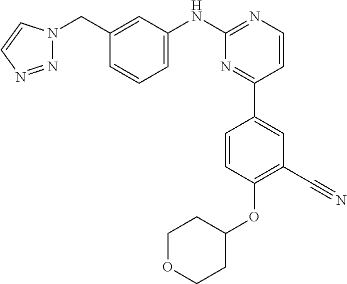 [SMILES: N#Cc1cc(-c2ccnc(Nc3cccc(Cn4ccnn4)c3)n2)ccc1OC1CCOCC1] | 2-(Tetrahydro-2H- pyran-4-yloxy)-5-(2- {[3-(1H-1,2,3- triazol-1- ylmethyl)phenyl] amino}pyrimidin-4- yl)benzonitrile | ¹H NMR (CDCl₃) δ 8.34 (s, 1H), 7.94 (m, 1H), 7.53 (d, 1H), 7.53 (d, 1H), 7.38 (t, 1H), 7.17-7.09 (m, 3H), 4.84-4.81 (m, 1H), 4.65 (s, 2H), 4.07-4.01 (m, 2H), 3.70-3.64 (m, 2H), 2.13-2.07 (m, 2H), 1.97- 1.90 (m, 2H). LC-MS [M + H]⁺ 454.2022 |
| 577 | 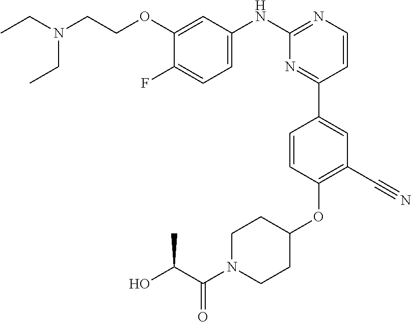 [SMILES: CCN(CC)CCOc1cc(Nc2nccc(-c3ccc(OC4CCN(C(=O)[C@H](C)O)CC4)c(C#N)c3)n2)ccc1F] | 5-[2-({3-[2- (Diethylamino)ethoxy]- 4- fluorophenyl}amino) pyrimidin-4-yl]-2- ({1-[(2S)-2- hydroxypropanoyl] piperidin-4- yl}oxy)benzonitrile | ¹H NMR (DMSO-d₆) δ ppm 9.79 (s, 1H), 9.40 (s, 1H), 8.55-8.60 (m, 2H), 8.45 (dd, 1H), 7.87 (d, 1H), 7.56 (d, 2H), 7.51 (d, 1H), 7.29- 7.37 (m, 2H), 7.17-7.26 (m, 2H), 5.01 (br. s., 1H), 4.39-4.50 (m, 4H), 3.75 (br. s., 2H), 3.59-3.65 (m, 2H), 3.22-3.33 (m, 5H), 2.00 (s, 2H), 1.72 (s, 2H), 1.22-1.29 (m, 6H); LC-MS [M + H]⁺ 577.2944. |
| 578 | 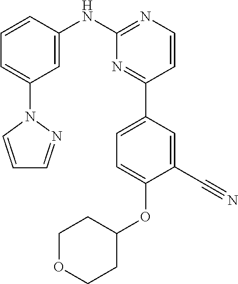 [SMILES: N#Cc1cc(-c2ccnc(Nc3cccc(-n4cccn4)c3)n2)ccc1OC1CCOCC1] | 5-(2-{[3-(1H- Pyrazol-1- yl)phenyl]amino} pyrimidin-4-yl)-2- (tetrahydro-2H- pyran-4- yloxy)benzonitrile | ¹H NMR (CDCl₃) δ 9.96 (s, 1H), 8.79-8.46 (m, 5H), 7.85 (d, 1H), 7.60-7.50 (m, 2H), 7.41-7.39 (m, 2H), 6.57 (m, 1H), 4.99 (m, 1H), 3.90-3.85 (m, 2H), 3.62-3.52 (m, 2H), 2.07-2.04 (m, 2H), 1.74-1.67 (m, 2H). LC-MS [M + H]⁺ 439.1888 |
| 579 | 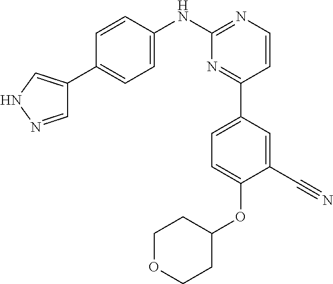 [SMILES: N#Cc1cc(-c2ccnc(Nc3ccc(-c4cn[nH]c4)cc3)n2)ccc1OC1CCOCC1] | 5-(2-{[4-(1H- Pyrazol-4- yl)phenyl]amino} pyrimidin-4-yl)-2- (tetrahydro-2H- pyran-4- yloxy)benzonitrile | ¹H NMR (CDCl₃) δ 9.70 (s, 1H), 8.58-8.55 (m, 2H), 8.48-8.45 (m, 2H), 8.00 (m, 2H), 7.80-7.78 (m, 2H), 7.58-7.56 (m, 3H), 7.48-7.46 (m, 1H), 4.95 (m, 1H), 3.91-3.85 (m, 2H), 3.59-3.53 (m, 2H), 2.07- 2.02 (m, 2H), 1.71-1.67 (m, 2H). LC-MS [M + H]⁺ 439.1939 |
| 580 | 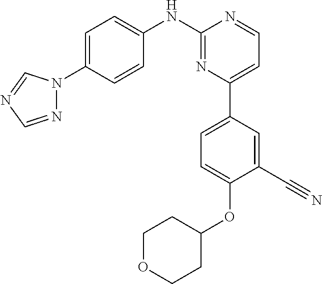 [SMILES: N#Cc1cc(-c2ccnc(Nc3ccc(-n4cncn4)cc3)n2)ccc1OC1CCOCC1] | 2-(Tetrahydro-2H- pyran-4-yloxy)-5-(2- {[4-(1H-1,2,4- triazol-1- yl)phenyl]amino} pyrimidin-4- yl)benzonitrile | ¹H NMR (MeOH-d₄) δ 9o.04 (s, 1H), 8.51-8.46 (m, 2H), 8.45-8.42 (m, 1H), 7.97 (d, 2H), 7.76 (d, 2H), 7.41-7.39 (m, 2H), 7.36 (s, 1H), 5.07-5.02 (m, 1H), 4.03-3.98 (m, 2H), 3.70-3.63 (m, 2H), 2.14-2.09 (m, 2H), 1.89-1.81 (m, 2H). LC-MS [M + H]⁺ 440.1838. |
| 581 | 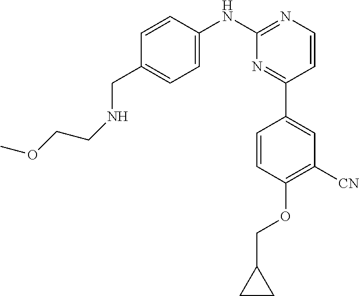 [SMILES: COCCNCc1ccc(Nc2nccc(-c3ccc(OCC4CC4)c(C#N)c3)n2)cc1] | 2- (Cyclopropylmethoxy)- 5-{2-[(4-{[(2- methoxyethyl)amino] methyl}phenyl)amino] pyrimidin-4- yl}benzonitrile | ¹H NMR (CDCl₃) δ 8.44 (d, 1H), \8.27-8.22 (m, 2H), 7.69 (d, 2H), 7.50 (d, 2H), 7.23-7.04 (m, 2H), 4.09 (s, 2H), 4.02 (d, 2H), 3.76- 3.73 (m, 2H), 3.39 (s, 3H), 3.08- 3.05 (m, 2H), 1.37-1.31 (m, 1H), 0.74-0.69 (m, 2H), 0.46-0.42 (m, 2H). LC-MS [M + H]⁺ 430.2237 |
| 582 | 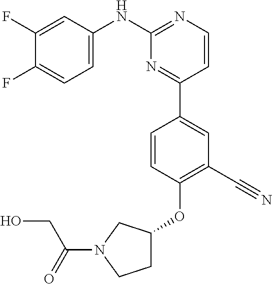 [SMILES: N#Cc1cc(-c2ccnc(Nc3ccc(F)c(F)c3)n2)ccc1O[C@@H]1CCN(C(=O)CO)C1] | 5-{2-[(3,4- Difluorophenyl)amino] pyrimidin-4-yl}-2- {[(3R)-1- (hydroxyacetyl) pyrrolidin-3- yl]oxy}benzonitrile | ¹H NMR (DMSO-d6) δ 9.96 (s, 1H), 8360-8.45 (m, 3H), 8.06-8.01 (m, 1H), 7.54-7.49 (m, 3H), 7.41- 7.34 (m, 1H), 5.45-5.30 (m, 1H), 4.73-4.68 (m, 1H), 4.09-4.00 (m, 2H), 3.66-3.59 (m, 2H), 3.17-3.10 (m, 2H), 2.51-2.15 (m, 2H). LC- MS [M + H]⁺ 452.1636 |
| 583 | 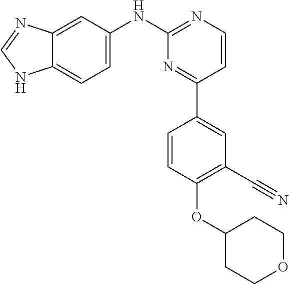 [SMILES: N#Cc1cc(-c2ccnc(Nc3ccc4[nH]cnc4c3)n2)ccc1OC1CCOCC1] | 5-[2-(1H- Benzimidazol-5- ylamino)pyrimidin- 4-yl]-2-(tetrahydro- 2H-pyran-4- yloxy)benzonitrile | ¹H NMR (DMSO-d₆) δ 10.1 (s, 1H), 9.30 (br s, 1H), 8.64-8.48 (m, 4H), 7.77 (br s, 2H), 7.57-7.54 (m, 2H), 4.97-4.94 (m, 1H), 3.91-3.85 (m, 2H), 3.59-3.55 (m, 2H), 2.08-2.01 (m, 2H), 1.73-1.66 (m, 2H); LC- MS [M + H]⁺ 413.1718 |
| 584 | 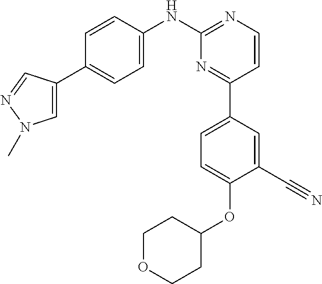 [SMILES: Cn1cc(-c2ccc(Nc3nccc(-c4ccc(OC5CCOCC5)c(C#N)c4)n3)cc2)cn1] | 5-(2-{[4-(1-Methyl- 1H-pyrazol-4- yl)phenyl]amino} pyrimidin-4-yl)-2- (tetrahydro-2H- pyran-4- yloxy)benzonitrile | ¹H NMR (CDCl₃) δ 9.72 (s, 1H), 8.56-8.55 (m, 2H), 8.48-8.45 (m, 1H), 8.06 (s, 1H), 7.81-7.78 (m, 3H), 7.58-7.47 (m, 3H), 4.97-4.92 (m, 1H), 3.91-3.81 (m, 5H), 3.59- 3.53 (m, 2H), 2.07-2.03 (m, 2H), 1.73-1.65 (m, 2H). LC-MS [M + H]⁺ 453.2072 |
| 585 | 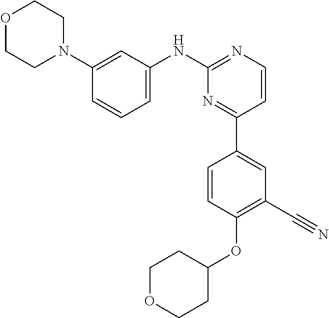 [SMILES: N#Cc1cc(-c2ccnc(Nc3cccc(N4CCOCC4)c3)n2)ccc1OC1CCOCC1] | 5-(2-{[3-(Morpholin- 4- yl)phenyl]amino} pyrimidin-4-yl)-2- (tetrahydro-2H- pyran-4- yloxy)benzonitrile | LC-MS [M + H]⁺ 458.2176 |
| 586 | 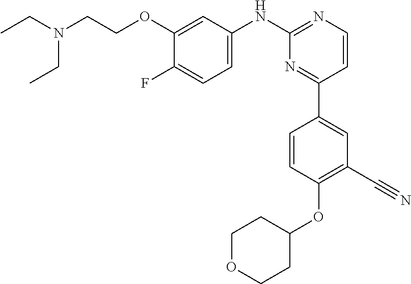 [SMILES: CCN(CC)CCOc1cc(Nc2nccc(-c3ccc(OC4CCOCC4)c(C#N)c3)n2)ccc1F] | 5-[2-({3-[2- (Diethylamino)ethoxy]- 4- fluorophenyl}amino) pyrimidin-4-yl]-2- (tetrahydro-2H- pyran-4- yloxy)benzonitrile | ¹H NMR (DMSO-d₆) δ ppm 9.70 (s, 1H), 8.50-8.58 (m, 2H), 8.45 (d, 1H), 7.75-7.85 (m, 1H), 7.53 (d, 1H), 7.48 (d, 1H), 7.24 (d, 2H), 7.08-7.19 (m, 1H), 4.87-4.97 (m, 1H), 4.04-4.12 (m, 2H), 3.82- 3.92 (m, 2H), 3.49-3.60 (m, 2H), 2.76-2.85 (m, 2H), 2.42-2.59 (m, 4H), 2.04 (d, 2H), 1.63-1.74 (m, 2H), 0.95 (t, 6H); LC-MS [M + H]⁺ 506.2499 |
| 587 | 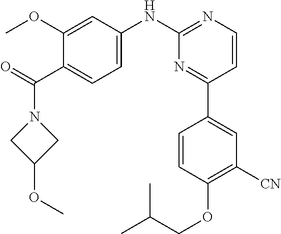 [SMILES: COc1cc(Nc2nccc(-c3ccc(OCC(C)C)c(C#N)c3)n2)ccc1C(=O)N1CC(OC)C1] | 5-[2-({3-Methoxy-4- [(3-methoxyazetidin- 1- yl)carbonyl]phenyl} amino)pyrimidin-4- yl]-2-(2- methylpropoxy) benzonitrile | ¹H NMR (CDCl₃) δ 8.49 (d, 1H), 8.37 (d, 1H), 8.22 (d, 1H), 7.79 (s, 1H), 7.39-7.31 (m, 2H), 7.12 (d, 1H), 7.07-7.02 (m, 2H), 4.36-4.33 (m, 1H), 4.23-4.15 (m, 2H), 4.07- 3.89 (m, 4H), 3.97 (s, 3H), 3.31 (s, 3H), 2.22-2.19 (m, 1H), 1.10 (d, 6H). LC-MS [M + H]⁺ 488.2271 |
| 588 | 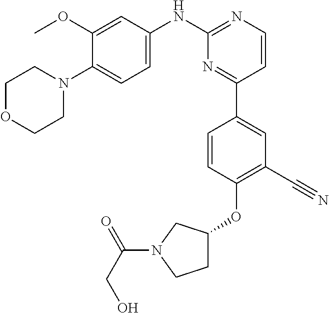 [SMILES: COc1cc(Nc2nccc(-c3ccc(O[C@@H]4CCN(C(=O)CO)C4)c(C#N)c3)n2)ccc1N1CCOCC1] | 2-{[(3R)-1- (hydroxyacetyl) pyrrolidin-3-yl]oxy}-5- (2-{[3-methoxy-4- (morpholin-4- yl)phenyl]amino} pyrimidin-4- yl)benzonitrile | ¹H NMR (DMSO-d6) δ 9.59 (s, 1H), 8.56 (d, 1H), 8.53 (d, 1H), 8.47 (d, 1H), 7.65 (s, 1H), 7.51 (d, 1H), 7.44 (d, 1H), 7.26 (d, 1H), 6.88 (br s, 1H), 5.44-5.20 (m, 1H), 4.11-3.96 (m, 2H), 3.89-3.39 (m, 12H), 3.84 (s, 3H), 2.32-2.11 (m, 2H). LC-MS [M + H]⁺ 531.2345 |
| 589 | 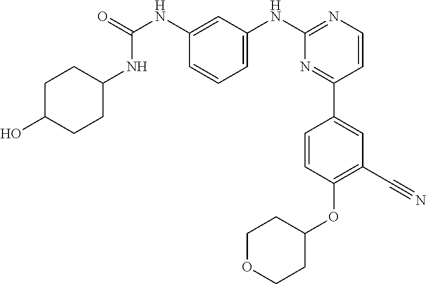 [SMILES: N#Cc1cc(-c2ccnc(Nc3cccc(NC(=O)NC4CCC(O)CC4)c3)n2)ccc1OC1CCOCC1] | 1-[3-({4-[3-Cyano-4- (tetrahydro-2H- pyran-4- yloxy)phenyl] pyrimidin-2- yl}amino)phenyl]-3- (4- hydroxycyclohexyl) urea | ¹H NMR (MeOH-d₄) δ 8.55 (d, 1H), 8.47 (d, 1H), 8.37 (d, 1H), 8.02 (br s, 1H), 7.39 (dd, 2H), 7.26 (t, 1H), 7.16 (d, 1H), 6.80 (d, 1H), 4.98- 4.90 (m, 1H), 4.03-3.96 (m, 2H), 3.70-3.52 (m, 4H), 2.16-1.80 (m, 9H), 1.44-1.24 (m, 4H). LC-MS [M + H]⁺ 529.2558 |
| 590 | 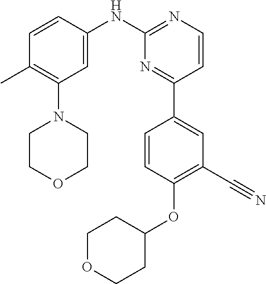 [SMILES: Cc1ccc(Nc2nccc(-c3ccc(OC4CCOCC4)c(C#N)c3)n2)cc1N1CCOCC1] | 5-(2-{[4-Methyl-3- (morpholin-4- yl)phenyl]amino} pyrimidin-4-yl)-2- (tetrahydro-2H- pyran-4- yloxy)benzonitrile | ¹H NMR (DMSO-d₆) δ 9.57 (s, 1H), 8.54-8.52 (m, 2H), 8.44 (dd, 1H), 7.64 (d, 1H), 7.53 (d, 1H), 7.43 (d, 1H), 7.33 (dd, 1H), 7.09 (d, 1H), 4.99-4.92 (m, 1H), 3.90-3.84 (m, 2H), 3.76 (t, 4H), 3.59-3.52 (m, 2H), 2.85 (t, 4H), 2.22 (s, 3H), 2.08-2.00 (m, 2H), 1.73-1.65 (m, 2H). LC-MS [M + H]⁺ 472.2352 |
| 591 | 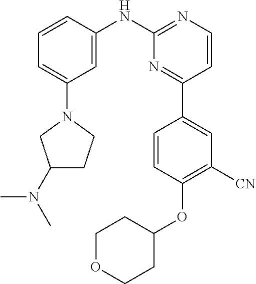 [SMILES: CN(C)C1CCN(c2cccc(Nc3nccc(-c4ccc(OC5CCOCC5)c(C#N)c4)n3)c2)C1] | 5-[2-({3-[3- (Dimethylamino) pyrrolidin-1- yl]phenyl}amino) pyrimidin-4-yl]-2- (tetrahydro-2H- pyran-4- yloxy)benzonitrile | ¹H NMR (CDCl₃) δ 8.46 (d, 1H), 8.38 (d, 1H), 8.24 (d, 1H), 7.24- 7.19 (m, 2H), 7.10-7.04 (m, 3H), 6.86 (d, 1H), 6.30 (d, 1H), 4.78- 4.73 (m, 1H), 4.07-4.02 (m, 2H), 3.69-3.65 (m, 2H), 3.57-3.50 (m, 2H), 3.44-3.38 (m, 1H), 3.23-3.19 (m, 1H), 2.93-2.89 (m, 1H), 2.33 (s, 6H), 2.31-2.24 (m, 1H), 2.13-2.02 (m, 2H), 1.97-1.88 (m, 3H). LC- MS [M + H]⁺ 485.2646 |
| 592 | 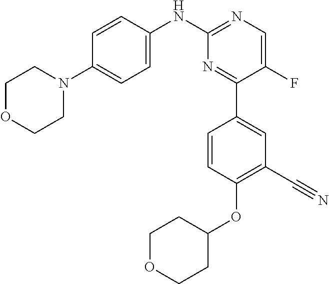 [SMILES: N#Cc1cc(-c2nc(Nc3ccc(N4CCOCC4)cc3)ncc2F)ccc1OC1CCOCC1] | 5-(5-Fluoro-2-{[4- (morpholin-4- yl)phenyl]amino} pyrimidin-4-yl)-2- (tetrahydro-2H- pyran-4- yloxy)benzonitrile | ¹H NMR (DMSO-d₆) δ ppm 9.55 (s, 1H), 8.58 (d, 1H), 8.26-8.34 (m, 2H), 7.52-7.62 (m, 3H), 6.86- 6.94 (m, 2H), 4.90-4.98 (m, 1H), 3.80-3.91 (m, 2H), 3.68-3.78 (m, 4H), 2.50-2.60 (m, 2H), 2.99- 3.06 (m, 4H), 1.99-2.09 (m, 2H), 1.65-1.75 (m, 2H); LC-MS [M + H]⁺ 476.2123 |
| 593 | 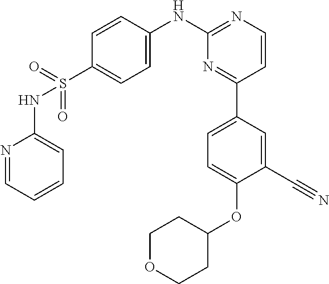 [SMILES: N#Cc1cc(-c2ccnc(Nc3ccc(S(=O)(=O)Nc4ccccn4)cc3)n2)ccc1OC1CCOCC1] | 4-({4-[3-Cyano-4- (tetrahydro-2H- pyran-4- yloxy)phenyl] pyrimidin-2-yl} amino)-N- (pyridin-2- yl) benzenesulfonamide | ¹H NMR (CDCl₃) δ 10.16 (s, 1H), 8.62 (d, 1H), 8.57 (d, 1H), 8.48 (m, 1H), 8.05 (s, 1H), 7.96 (m, 2H), 7.83 (m, 2H), 7.70 (m, 1H), 7.59- 7.56 (m, 1H), 7.14 (m, 1H), 6.88 (m, 1H), 4.95 (m, 1H), 3.91-3.85 (m, 2H), 3.59-3.51 (m, 2H), 2.09- 2.03 (m, 2H), 1.74-1.65 (m, 2H). LC-MS [M + H]⁺ 529.1688 |
| 594 | 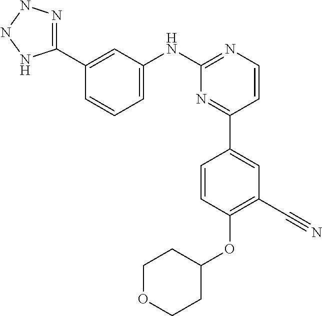 [SMILES: N#Cc1cc(-c2ccnc(Nc3cccc(C4=NNNN4)c3)n2)ccc1OC1CCOCC1] | 2-(Tetrahydro-2H- pyran-4-yloxy)-5-(2- {[3-(1H-tetrazol-5- yl)phenyl]amino} pyrimidin-4- yl)benzonitrile | ¹H NMR (CDCl₃) δ 9.86 (s, 1H), 8.60-8.55 (m, 4H), 7.81 (d, 1H), 7.60 (d, 1H), 7.55-7.51 (m, 2H), 7.41 (t, 1H), 4.98-4.92 (m, 1H), 3.90-3.85 (m, 2H), 3.59-3.54 (m, 2H), 2.07-2.03 (m, 2H), 1.74-1.65 (m, 2H). LC-MS [M + H]⁺ 441.1752 |
| 595 | 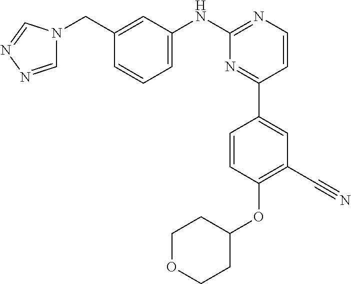 [SMILES: N#Cc1cc(-c2ccnc(Nc3cccc(Cn4cnnc4)c3)n2)ccc1OC1CCOCC1] | 2-(Tetrahydro-2H- pyran-4-yloxy)-5-(2- {[3-(4H-1,2,4- triazol-4- ylmethyl)phenyl] amino}pyrimidin-4- yl)benzonitrile | ¹H NMR (CDCl₃) δ 8.39-8.31 (m, 3H), 7.94 (m, 1H), 7.53 (d, 1H), 7.38 (t, 1H), 7.16-7.09 (m, 3H), 4.82-4.74 (m, 1H), 4.65 (s, 2H), 4.07-4.01 (m, 2H), 3.70-3.64 (m, 2H), 2.13-2.07 (m, 2H), 1.97-1.90 (m, 2H). LC-MS [M + H]⁺ 454.1998 |
| 596 | 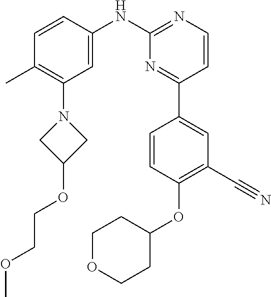 [SMILES: COCCOC1CN(c2cc(Nc3nccc(-c4ccc(OC5CCOCC5)c(C#N)c4)n3)ccc2C)C1] | 5-[2-({3-[3-(2- Methoxyethoxy) azetidin-1-yl]-4- methylphenyl} amino) pyrimidin-4-yl]-2- (tetrahydro-2H- pyran-4- yloxy)benzonitrile | ¹H NMR (DMSO-d₆) δ 9.48 (s, 1H), 8.53 (d, 1H), 8.52 (d, 1H), 8.44 (dd, 1H), 7.54 (d, 1H), 7.42 (d, 1H), 7.14 (s, 1H), 7.10 (dd, 1H), 6.91 (d, 1H), 4.99-4.92 (m, 1H), 4.39-4.34 (m, 1H), 4.13 (t, 2H), 3.90-3.84 (m, 2H), 3.63-3.60 (m, 2H), 3.58-3.53 (m, 4H), 3.46-3.44 (m, 2H), 3.24 (s, 3H), 2.10 (s, 3H), 2.07-2.00 (m, 2H), 1.73-1.65 (m, 2H). LC-MS [M + H]⁺ 516.2616 |
| 597 | 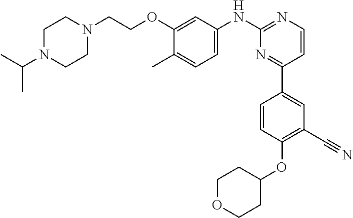 [SMILES: Cc1ccc(Nc2nccc(-c3ccc(OC4CCOCC4)c(C#N)c3)n2)cc1OCCN1CCN(C(C)C)CC1] | 5-{2-[(4-Methyl-3- {2-[4-(propan-2- yl)piperazin-1- yl]ethoxy}phenyl) amino]pyrimidin- 4-yl}- 2-(tetrahydro-2H- pyran-4- yloxy)benzonitrile | ¹H NMR (DMSO-d₆) δ ppm 9.63 (s, 1H), 9.29 (s, 1H), 8.51-8.58 (m, 2H), 8.44 (dd, 1H), 7.63 (s, 1H), 7.55 (d, 1H), 7.46 (d, 1H), 7.21 (d, 1H), 7.06 (d, 1H), 4.89-4.99 (m, 1H), 4.15 (s, 2H), 3.82-3.91 (m, 2H), 3.50-3.61 (m, 2H), 3.39 (d, 4H), 3.11-3.23 (m, 3H), 2.82- 3.09 (m, 4H), 2.13 (s, 3H), 2.00- 2.10 (m, 2H), 1.63-1.74 (m, 2H), 1.24 (d, 6H); LC-MS [M + H]⁺ 557.3213 |
| 598 | 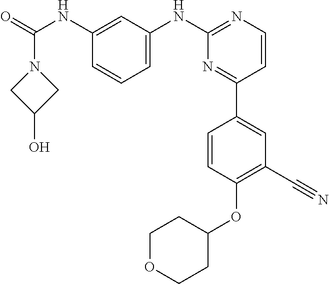 [SMILES: N#Cc1cc(-c2ccnc(Nc3cccc(NC(=O)N4CC(O)C4)c3)n2)ccc1OC1CCOCC1] | N-[3-({4-[3-Cyano- 4-(tetrahydro-2H- pyran-4- yloxy)phenyl] pyrimidin-2- yl}amino)phenyl]-3- hydroxyazetidin-1- carboxamide | ¹H NMR (DMSO-d₆) δ 9.61 (s, 1H), 8.61-8.53 (m, 3H), 8.40 (s, 1H), 7.51-7.45 (m, 2H), 7.22 (d, 1H), 7.13 (t, 1H), 7.04-7.02 (m, 2H), 4.98-4.90 (m, 1H), 4.44-4.38 (m, 1H), 4.17-4.10 (m, 2H), 3.91-3.85 (m, 2H), 3.74-3.71 (m, 2H), 3.58- 3.53 (m, 2H), 3.17 (d, 1H), 2.10- 2.01 (m, 2H), 1.75-1.64 (m, 2H). LC-MS [M + H]⁺ 487.2060 |
| 599 | 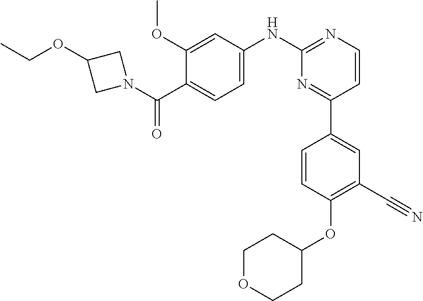 [SMILES: CCOC1CN(C(=O)c2ccc(Nc3nccc(-c4ccc(OC5CCOCC5)c(C#N)c4)n3)cc2OC)C1] | 5-[2-({4-[(3- Ethoxyazetidin-1- yl)carbonyl]-3- methoxyphenyl} amino)pyrimidin- 4-yl]-2- (tetrahydro-2H- pyran-4- yloxy)benzonitrile | ¹H NMR (DMSO-d₆) δ 9.95 (s, 1H), 8.61 (d, 1H), 8.59 (d, 1H), 8.46 (dd, 1H), 7.85 (s, 1H), 7.57-7.54 (m, 2H), 7.32 (d, 1H), 7.26 (d, 1H), 4.99-4.92 (m, 1H), 4.32-4.27 (m, 1H), 4.19-4.15 (m, 1H), 4.09-4.05 (m, 1H), 3.90-3.83 (m, 2H), 3.88 (s, 3H), 3.79-3.74 (m, 2H), 3.59-3.54 (m, 2H), 3.42-3.36 (m, 2H), 2.09- 2.02 (m, 2H), 1.73-1.65 (m, 2H), 1.12 (t, 3H). LC-MS [M + H]⁺ 530.240 |
| 600 | 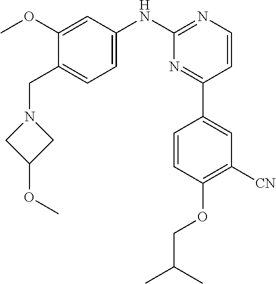 [SMILES: COc1cc(Nc2nccc(-c3ccc(OCC(C)C)c(C#N)c3)n2)ccc1CN1CC(OC)C1] | 5-[2-({3-Methoxy-4- [(3-methoxyazetidin- 1- yl)methyl]phenyl} amino)pyrimidin- 4-yl]- 2-(2- methylpropoxy) benzonitrile | ¹H NMR (CDCl₃) δ 8.74-8.63 (m, 3H), 7.56 (s, 1H), 7.26-7.15 (m, 4H), 5.35 (s, 1H), 4.05-3.99 (m, 5H), 3.84-3.79 (m, 1H), 3.67-3.40 (m, 3H), 3.39-3.27 (m, 4H), 3.22 (s, 6H), 2.47-2.37 (m, 2H). LC-MS [M + H]⁺ 474.2140 |
| 601 | 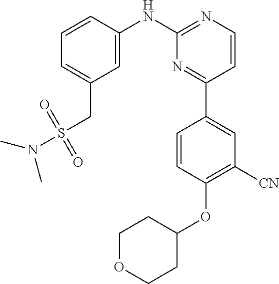 [SMILES: CN(C)S(=O)(=O)Cc1cccc(Nc2nccc(-c3ccc(OC4CCOCC4)c(C#N)c3)n2)c1] | 1-[3-({4-[3-Cyano-4- (tetrahydro-2H- pyran-4- yloxy)phenyl] pyrimidin-2- yl}amino)phenyl]- N,N- dimethylmethane- sulfonamide | ¹H NMR (CDCl₃) δ 8.48 (d, 1H), 8.36-8.29 (m, 2H), 7.94 (s, 1H), 7.61 (d, 1H), 7.41-7.35 (m, 1H), 7.24-7.06 (m, 3H), 4.81-4.76 (m, 1H), 4.33-4.23 (m, 2H), 4.06-4.02 (m, 2H), 3.70-3.66 (m, 2H), 2.79 (s, 6H), 2.12-2.05 (m, 2H), 1.94-1.89 (m, 2H). LC-MS [M + H]⁺ 494.1883 |
| 602 | 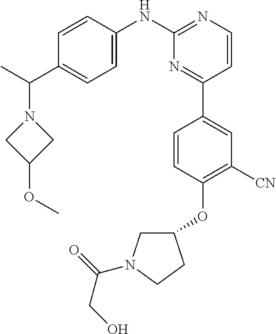 [SMILES: COC1CN(C(C)c2ccc(Nc3nccc(-c4ccc(O[C@@H]5CCN(C(=O)CO)C5)c(C#N)c4)n3)cc2)C1] | 2-{[(3R)-1- (Hydroxyacetyl) pyrrolidin-3-yl]oxy}-5- [2-({4-[1-(3- methoxyazetidin-1- yl)ethyl]phenyl}amino) pyrimidin-4- yl]benzonitrile] | ¹H NMR (CDCl₃) δ 8.54-8.48 (m, 2H), 8.41 (d, 1H), 7.89 (d, 2H), 7.41-7.32 (m, 5H), 5.41-5.32 (m, 1H), 4.44-4.36 (m, 1H), 4.25-4.16 (m, 3H), 3.88-3.71 (m, 4H), 3.39 (s, 3H), 2.44-2.28 (m, 2H), 1.61 (s, 3H), 1.35-1.28 (m, 4H). LC-MS [M + H]⁺ 529.2575 |
| 603 | 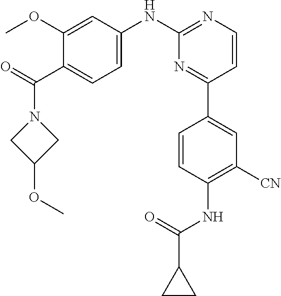 [SMILES: COc1cc(Nc2nccc(-c3ccc(NC(=O)C4CC4)c(C#N)c3)n2)ccc1C(=O)N1CC(OC)C1] | N-{2-Cyano-4-[2- ({3-methoxy-4-[(3- methoxyazetidin-1- yl)carbonyl]phenyl} amino)pyrimidin-4- yl]phenyl} cyclopropane- carboxamide | ¹H NMR (CDCl₃) δ 8.59 (d, 1H), 8.52 (d, 1H), 8.39 (d, 1H), 8.24- 8.22 (m, 1H), 8.05 (s, 1H), 7.79 (d, 1H), 7.40 (s, 1H), 7.16 (d, 1H), 4.39-4.34 (m, 1H), 4.27-4.21 (m, 1H), 4.17-4.13 (m, 1H), 4.07-4.04 (m, 1H), 4.00-3.91 (m, 1H), 3.96 (s, 3H), 3.31 (s, 3H), 1.7-1.165 (m, 1H), 1.19-1.17 (m, 2H), 1.03-0.99 (m, 2H). LC-MS [M + H]⁺ 499.2093 |
| 604 | 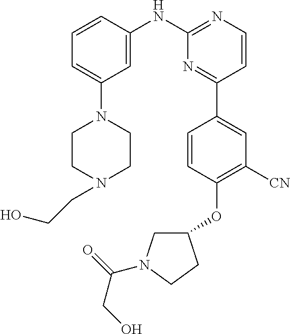 [SMILES: N#Cc1cc(-c2ccnc(Nc3cccc(N4CCN(CCO)CC4)c3)n2)ccc1O[C@@H]1CCN(C(=O)CO)C1] | 2-{[(3R)-1- (Hydroxyacetyl) pyrrolidin-3-yl] oxy}-5- [2-({3-[4-(2- hydroxyethyl) piperazin-1- yl]phenyl}amino) pyrimidin-4- yl]benzonitrile | ¹H NMR (MeOH-d₄) δ 8.45-8.39 (m, 3H), 7.64-7.63 (m, 1H), 7.33 (d, 1H), 7.24-7.18 (m, 2H), 7.10- 7.08 (m, 1H), 6.67 (d, 1H), 5.36- 5.30 (m, 1H), 4.24-4.17 (m, 2H), 3.89-3.62 (m, 6H), 3.36-3.27 (m, 4H), 2.78-2.73 (m, 4H), 2.68-2.61 (m, 2H), 2.41-2.30 (m, 2H). LC- MS [M + H]⁺ 544.2665 |
| 605 | 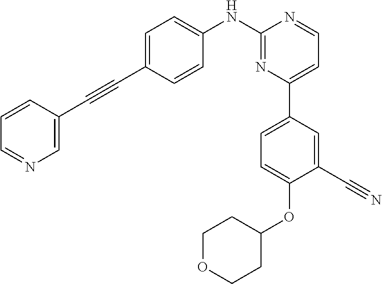 [SMILES: N#Cc1cc(-c2ccnc(Nc3ccc(C#Cc4cccnc4)cc3)n2)ccc1OC1CCOCC1] | 5-(2-{[4-(Pyridin-3- ylethynyl)phenyl] amino}pyrimidin-4-yl)- 2-(tetrahydro-2H- pyran-4- yloxy)benzonitrile | ¹H NMR (CD3-OD) δ 8.82 (bs, 1H), 8.61-8.54 (m, 1H), 8.49-8.44 (m, 2H), 8.41 (dd, 2H), 8.25- 8.21 (m, 1H), 7.84-7.83 (d, 1H), 7.81-7.75 (m, 1H), 7.72-7.62 (m, 2H), 7.59 (d, 1H), 7.44-7.34 (m, 2H), 4.90-4.84 (m, 1H), 4.02-3.94 (m, 2H), 3.69-3.60 (m, 2H), 2.14- 2.06 (m, 2H), 1.88-1.79 (m, 2H). LC-MS [M + H]⁺ 474.1916 |
| 606 | 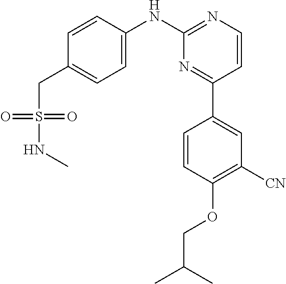 [SMILES: CNS(=O)(=O)Cc1ccc(Nc2nccc(-c3ccc(OCC(C)C)c(C#N)c3)n2)cc1] | 1-[4-({4-[3-Cyano-4- (2- methylpropoxy) phenyl]pyrimidin-2- yl}amino)phenyl]-N- methylmethane- sulfonamide | ¹H NMR (CDCl₃) δ 8.43 (d, 1H), 8.26-8.23 (m, 2H), 7.70 (d, 2H), 7.39 (d, 2H), 7.10-7.07 (m, 2H), 4.26 (s, 2H), 3.93 (d, 2H), 2.73 (s, 3H), 2.25-2.19 (m, 1H), 1.10 (d, 6H). LC-MS [M + H]⁺ 452.1707 |
| 607 | 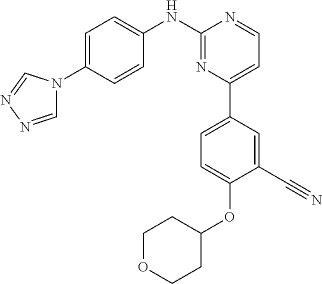 [SMILES: N#Cc1cc(-c2ccnc(Nc3ccc(-n4cnnc4)cc3)n2)ccc1OC1CCOCC1] | 2-(Tetrahydro-2H- pyran-4-yloxy)-5-(2- {[4-(4H-1,2,4- triazol-4- yl)phenyl]amino} pyrimidin-4- yl)benzonitrile | ¹H NMR (DMSO) 9.98 (s, 1H), 9.08 (s, 2H), 8.60 (d, 1H), 8.60 (d, 1H), 8.46 (dd, 1H), 8.0-7.97 (m, 2H), 7.66-7.62 (m, 2H), 7.65-7.53 (m, 3H), 4.99-4.93 (m, 1H), 3.91- 3.85 (m, 2H), 3.59-3.53 (m, 2H), 2.07-2.03 (m, 2H), 1.74-1.65 (m, 2H). δ LC-MS [M + H]⁺ 440.1843. |
| 608 | 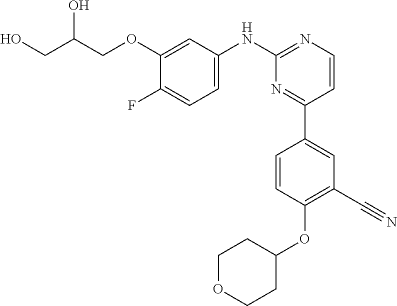 [SMILES: N#Cc1cc(-c2ccnc(Nc3ccc(F)c(OCC(O)CO)c3)n2)ccc1OC1CCOCC1] | 5-(2-{[3-(2,3- Dihydroxypropoxy)- 4- fluorophenyl]amino} pyrimidin-4-yl)-2- (tetrahydro-2H- pyran-4- yloxy)benzonitrile | LC-MS [M + H]⁺ 481.1889 |
| 609 | 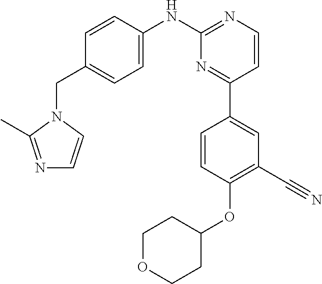 [SMILES: Cc1nccn1Cc1ccc(Nc2nccc(-c3ccc(OC4CCOCC4)c(C#N)c3)n2)cc1] | 5-[2-({4-[(2-Methyl- 1H-imidazol-1- yl)methyl]phenyl} amino)pyrimidin-4-yl]- 2-(tetrahydro-2H- pyran-4- yloxy)benzonitrile | ¹H NMR (DMSO-d₆) δ 9.74 (s, 1H), 8.55-8.53 (m, 2H), 8.44 (dd, 1H), 7.78 (d, 2H), .55 (d, 1H), 7.48 (d, 1H), 7.13-7.11 (m, 3H), 6.75 (d, 1H), 5.07 (s, 2H), 4.99-4.92 (m, 1H), 3.90-3.85 (m, 2H), 3.58-3.53 (m, 2H), 2.25 (s, 3H), 2.07-2.02 (m, 2H), 1.73-1.67 (m, 2H). LC-MS [M + H]⁺ 467.220 |
| 610 | 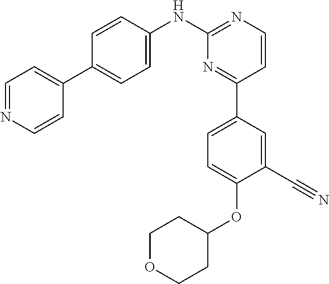 [SMILES: N#Cc1cc(-c2ccnc(Nc3ccc(-c4ccncc4)cc3)n2)ccc1OC1CCOCC1] | 5-(2-{[4-(Pyridin-4- yl)phenyl]amino} pyrimidin-4-yl)-2- (tetrahydro-2H- pyran-4- yloxy)benzonitrile | ¹H NMR (CDCl₃) δ 10.18 (s, 1H), 8.79 (m, 2H), 8.62 (m, 2H), 8.50 (m, 1H), 8.16 (m, 2H), 8.08-7.98 (m, 3H), 7.60-7.56 (m, 2H), 4.96 (m, 1H), 3.91-3.86 (m, 2H), 3.59- 3.53 (m, 2H), 2.08-2.03 (m, 2H), 1.74-1.66 (m, 2H). LC-MS [M + H]⁺ 450.1923 |
| 611 | 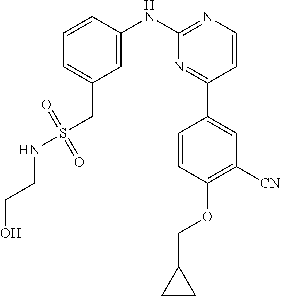 [SMILES: N#Cc1cc(-c2ccnc(Nc3cccc(CS(=O)(=O)NCCO)c3)n2)ccc1OCC1CC1] | 1-[3-({4-[3-Cyano-4- (cyclopropylmethoxy) phenyl]pyrimidin-2- yl}amino)phenyl]-N- (2- hydroxyethyl) methanesulfonamide | ¹H NMR (CDCl₃) δ 8.44 (d, 1H), 8.39 (s, 1H), 8.27-8.22 (m, 2H), 7.89 (s, 1H), 7.60 (d, 1H), 7.40- 7.37 (m, 1H), 7.12-7.05 (m, 2H), 4.33 (s, 2H), 4.03 (d, 2H), 3.64- 3.61 (m, 2H), 3.18-3.16 (m, 2H), 1.39-1.31 (m, 1H), 0.74-0.69 (m, 2H), 0.46-0.42 (m, 2H). LC-MS [M + H]⁺ 480.1717 |
| 612 | 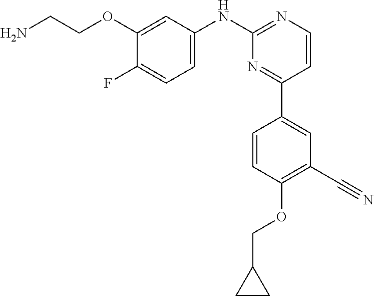 [SMILES: N#Cc1cc(-c2ccnc(Nc3ccc(F)c(OCCN)c3)n2)ccc1OCC1CC1] | 5-(2-{[3-(2- Aminoethoxy)-4- fluorophenyl]amino} pyrimidin-4-yl)-2- (cyclopropylmethoxy) benzonitrile | ¹H NMR (DMSO-d₆) δ ppm 9.78 (s, 1H), 8.53-8.57 (m, 2H), 8.45 (dd, 1H), 8.01 (br. s., 3H), 7.82-7.90 (m, 1H), 7.50 (d, 1H), 7.42 (d, 1H), 7.28-7.36 (m, 1H), 7.17-7.25 (m, 1H), 4.25-4.32 (m, 2H), 4.12 (d, 2H), 3.27-3.37 (m, 2H), 1.26- 1.37 (m, 1H), 0.58-0.68 (m, 2H), 0.37-0.45 (m, 2H); LC-MS [M + H]⁺ 420.1830 |
| 613 | 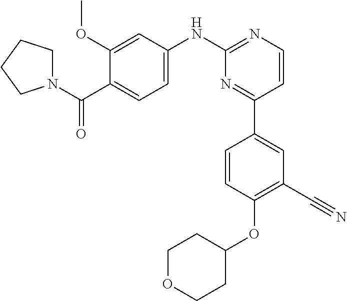 [SMILES: COc1cc(Nc2nccc(-c3ccc(OC4CCOCC4)c(C#N)c3)n2)ccc1C(=O)N1CCCC1] | 5-(2-{[3-Methoxy-4- (pyrrolidin-1- ylcarbonyl)phenyl] amino}pyrimidin-4- yl)-2-(tetrahydro-2H- pyran-4- yloxy)benzonitrile | ¹H NMR (DMSO-d₆) δ 9.89 (s, 1H), 8.60-8.58 (m, 2H), 8.46 (dd, 1H), \7.84 (s, 1H), 7.55 (d, 1H), 7.52 (d, 1H), 7.31 (d, 1H), 7.13 (d, 1H), 4.99-4.92 (m, 1H), 3.90-3.85 (m, 2H), 3.85 (s, 3H), 3.59-3.53 (m, 2H), 3.42 (t, 2H), 3.18 (t, 2H), 2.09-2.00 (m, 2H), 1.88-1.82 (m, 2H), 1.81-1.75 (m, 2H), 1.73-1.64 (m, 2H). LC-MS [M + H]⁺ 500.2292 |
| 614 | 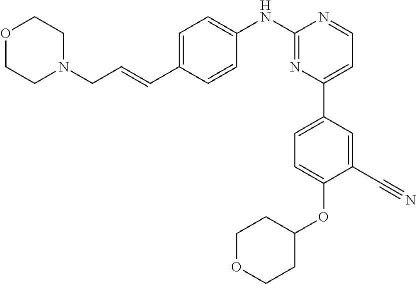 [SMILES: N#Cc1cc(-c2ccnc(Nc3ccc(/C=C/CN4CCOCC4)cc3)n2)ccc1OC1CCOCC1] | 5-[2-({4-[(1E)-3- (Morpholin-4- yl)prop-1-en-1- yl]phenyl}amino) pyrimidin-4-yl]-2- (tetrahydro-2H- pyran-4- yloxy)benzonitrile | ¹H NMR (CD3-OD) δ 8.49-8.43 (m, 2H), 8.36 (dd, 1H), 7.76 (d, 2H), 7.50 (d, 2H), 7.37-7.31 (m, 2H), 6.90 (d, 1H), 6.23 (dt, 1H), 4.92- 4.85 (m, 1H), 4.09-3.97 (m, 6H), 3.79-3.72 (m, 2H), 3.67-3.63 (m, 2H), 3.53-3.50 (m, 2H), 3.20-3.15 (m, 2H), 2.13-2.07 (m, 2H), 1.86- 81 (m, 2H). LC-MS [M + H]⁺ 498.2517. |
| 615 | 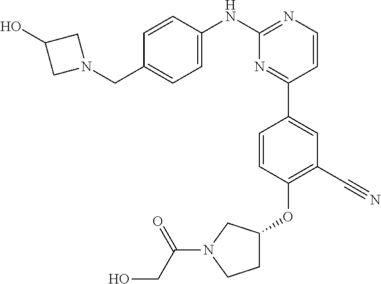 [SMILES: N#Cc1cc(-c2ccnc(Nc3ccc(CN4CC(O)C4)cc3)n2)ccc1O[C@@H]1CCN(C(=O)CO)C1] | 2-{[(3R)-1- (Hydroxyacetyl) pyrrolidin-3-yl]oxy}-5- [2-({4-[(3- hydroxyazetidin-1- yl)methyl]phenyl} amino)pyrimidin-4- yl]benzonitrile | ¹H NMR (DMSO-d₆) δ ppm 10.10 (br. s., 1H), 9.91 (br. s., 1H), 9.69 (br. s., 1H), 8.60 (d, 1H), 8.56 (d, 1H), 8.46-8.51 (m, 1H), 7.86- 7.92 (m, 2H), 7.49-7.56 (m, 2H), 7.42 (d, 2H), 5.39-5.46 (m, 1H), 5.31-5.38 (m, 1H), 4.58-4.69 (m, 1H), 4.40-4.48 (m, 1H), 4.27- 4.33 (m, 2H), 4.15-4.25 (m, 2H), 4.04-4.09 (m, 1H), 3.98-4.03 (m, 1H), 3.80-3.91 (m, 2H), 3.60- 3.69 (m, 2H), 3.42-3.53 (m, 1H), 2.23-2.34 (m, 1H), 2.12-2.23 (m, 1H); LC-MS [M + H]⁺ 501.2319 |
| 616 | 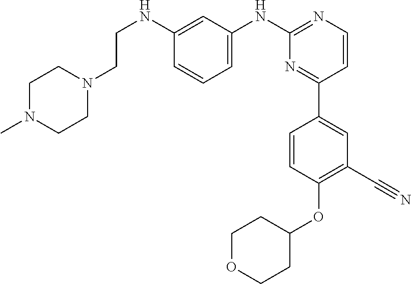 [SMILES: CN1CCN(CCNc2cccc(Nc3nccc(-c4ccc(OC5CCOCC5)c(C#N)c4)n3)c2)CC1] | 5-{2-[(3-{[2-(4- Methylpiperazin-1- yl)ethyl]amino} phenyl)amino] pyrimidin- 4-yl}-2-(tetrahydro- 2H-pyran-4- yloxy)benzonitrile | ¹H NMR (CDCl₃) δ 8.35 (d, 1H), 8.35 (s, 1H), 8.18 (d, 1H), 7.21 (t, 1H), 7.03 (d, 1H), 6.90 (d, 1H), 6.71 (d, 1H), 6.68-6.67 (m, 1H), 6.61 (d, 1H), 4.75-4.70 (m, 1H) 4.17 (t, 2H), 4.07-4.01 (m, 2H), 3.70-3.63 (m, 4H), 2.72 (t, 2H), 2.57-2.33 (m, 6H), 2.28 (s, 3H), 2.10-2.03 (m, 2H), 1.94-1.90 (m, 2H). 514.2899C-MS [M + H]⁺ |
| 617 | 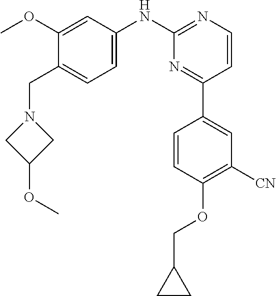 [SMILES: COc1cc(Nc2nccc(-c3ccc(OCC4CC4)c(C#N)c3)n2)ccc1CN1CC(OC)C1] | 2- (Cyclopropylmethoxy)- 5-[2-({3-methoxy- 4-[(3- methoxyazetidin-1- yl)methyl]phenyl} amino)pyrimidin-4- yl]benzonitrile | ¹H NMR (CDCl₃) δ 8.48 (d, 1H), 8.37 (d, 1H), 8.25 (d, 1H), 7.80 (s, 1H), 7.36-7.29 (m, 2H), 7.15-7.05 (m, 3H), 4.47-4.46 (m, 2H), 4.33- 4.24 (m, 3H), 4.05-3.98 (m, 2H), 3.l96 (s, 3H), 3.78-3.72 (m, 2H), 3.31 (s, 3H), 1.38-1.35 (m, 1H), 0.74-0.71 (m, 2H), 0.45-0.43 (m, 2H). LC-MS [M + H]⁺ 472.2197 |
| 618 | 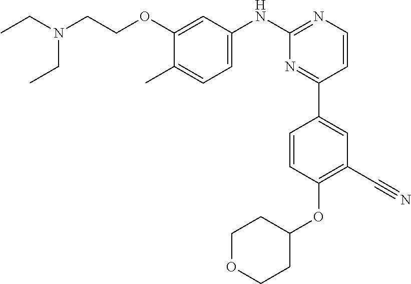 [SMILES: CCN(CC)CCOc1cc(Nc2nccc(-c3ccc(OC4CCOCC4)c(C#N)c3)n2)ccc1C] | 5-[2-({3-[2- (Diethylamino) ethoxy]-4- methylphenyl}amino) pyrimidin-4-yl]-2- (tetrahydro-2H- pyran-4- yloxy)benzonitrile | ¹H NMR (DMSO-d₆) δ ppm 9.67 (s, 1H), 9.39 (s, 1H), 8.54-8.63 (m, 2H), 8.44 (dd, 1H), 7.65 (d, 1H), 7.55 (d, 1H), 7.47 (d, 1H), 7.27 (dd, 1H), 7.10 (d, 1H), 4.89-4.98 (m, 1H), 4.29-4.39 (m, 2H), 3.81- 3.91 (m, 2H), 3.61-3.65 (m, 2H), 3.53-3.59 (m, 2H), 3.23-3.34 (m, 4H), 2.16 (s,, 3H), 1.98-2.09 (m, 2H), 1.64-1.74 (m, 2H), 1.28 (t, 6H); ), 9.3LC-MS [M + H]⁺ 502.2798 |
| 619 | 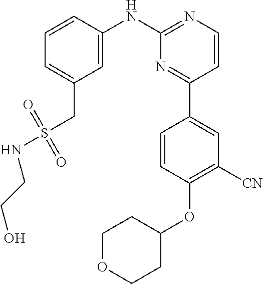 [SMILES: N#Cc1cc(-c2ccnc(Nc3cccc(CS(=O)(=O)NCCO)c3)n2)ccc1OC1CCOCC1] | 1-[3-({4-[3-Cyano-4- (tetrahydro-2H- pyran-4- yloxy)phenyl] pyrimidin-2- yl}amino)phenyl]-N- (2-hydroxyethyl) methanesulfonamide | ¹H NMR (CDCl₃) δ 8.43 (d, 1H), 8.37-8.27 (m, 2H), 7.89 (s, 1H), 7.59 (d, 1H), 7.38-7.34 (m, 1H), 7.17-7.09 (m, 3H), 6.79-6.69 (m, 2H), 4.78-4.77 (m, 1H), 4.41-4.26 (m, 2H), 4.22-4.15 (m, 2H), 4.07- 4.01 (m, 2H), 3.71-3.67 (m, 2H), 3.20-3.16 (m, 2H), 2.14-2.07 (m, 2H), 1.95-1.91 (m, 2H). LC-MS [M + H]⁺ 510.1806 |
| 620 | 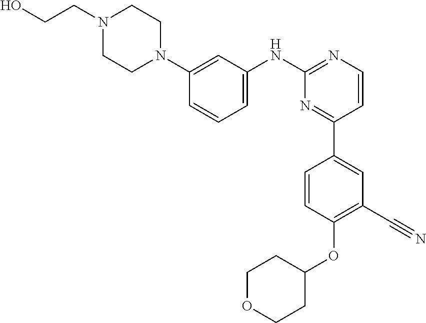 [SMILES: N#Cc1cc(-c2ccnc(Nc3cccc(N4CCN(CCO)CC4)c3)n2)ccc1OC1CCOCC1] | 5-[2-({3-[4-(2- Hydroxyethyl) piperazin-1- yl]phenyl}amino) pyrimidin-4-yl]-2- (tetrahydro-2H- pyran-4- yloxy)benzonitrile | ¹H NMR (CDCl₃) δ 8.47 (d, 1H), 8.39 (d, 1H), 8.21 (dd, 1H), 7.53- 7.52 (m, 1H), 7.20 (s, 1H), 7.08- 7.06 (m, 2H), 7.02-7.00 (m, 1H), 6.67 (dd, 1H), 4.78-4.74 (m, 1H), 4.07-4.01 (m, 2H), 3.69-3.64 (m, 5H), 3.30-3.28 (m, 4H), 2.74-2.71 (m, 4H), 2.11-2.07 (m, 2H), 1.95- 1.91 (m, 2H). LC-MS [M + H]⁺ 501.2531 |
| 621 | 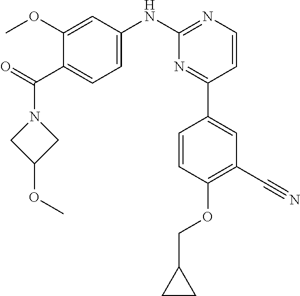 [SMILES: COc1cc(Nc2nccc(-c3ccc(OCC4CC4)c(C#N)c3)n2)ccc1C(=O)N1CC(OC)C1] | 2- (Cyclopropylmethoxy)- 5-[2-({3-methoxy- 4-[(3- methoxyazetidin-1- yl)carbonyl]phenyl} amino)pyrimidin-4- yl]benzonitrile | ¹H NMR (CDCl₃) δ 8.50 (d, 1H), 8.37 (d, 1H), 8.22 (d, 1H), 7.79 (s, 1H), 7.44-7.31 (m, 2H), 7.13 (d, 1H), 7.09-6.99 (m, 2H), 4.36-4.33 (m, 1H), 4.23-4.15 (m, 2H), 4.07- 3.92 (m, 4H), 3.96 (s, 3H), 3.31 (s, 3H), 1.37-1.31 (m, 1H), 0.74-0.71 (m, 2H), 0.48-0.41 (m, 2H). LC- MS [M + H]⁺ 486.2117 |
| 622 | 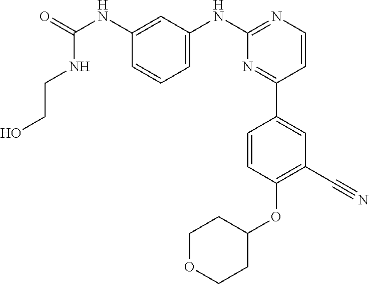 [SMILES: N#Cc1cc(-c2ccnc(Nc3cccc(NC(=O)NCCO)c3)n2)ccc1OC1CCOCC1] | 1-[3-({4-[3-Cyano-4- (tetrahydro-2H- pyran-4- yloxy)phenyl] pyrimidin-2- yl}amino)phenyl]-3- (2-hydroxyethyl)urea | ¹H NMR (MeOH-d₄) δ 8.50-8.42 (m, 3H), 8.08 (m, 1H), 7.34 (d, 1H), 7.27-7.17 (m, 4H), 6.92 (d, 1H), 4.98-4.90 (m, 1H), 4.12-3.96 (m, 2H), 3.69-3.62 (m, 5H), 3.35 (t, 2H), 2.15-2.07 (m, 2H), 1.88-1.79 (m, 2H). LC-MS [M + H]⁺ 475.2083 |
| 623 | 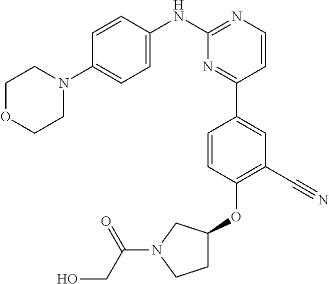 [SMILES: N#Cc1cc(-c2ccnc(Nc3ccc(N4CCOCC4)cc3)n2)ccc1O[C@H]1CCN(C(=O)CO)C1] | 2-{[(3S)-1- (Hydroxyacetyl) pyrrolidin-3-yl] oxy}-5- (2-{[4-(morpholin-4- yl)phenyl]amino} pyrimidin-4- yl)benzonitrile | ¹H NMR (DMSO-d₆) δ 9.47 (s, 1H), 8.53 (d, 1H), 8.49 (d, 1H), 8.48- 8.44 (m, 1H), 7.66-7.62 (m, 2H), 7.51 (dd, 1H), 7.40 (dd, 1H), 6.94- 6.92 (m, 2H), 5.41 (br s, 0.47H), 5.33 (br s, 0.53 H), 4.72-4.67 (m, 1H), 4.13-3.95 (m, 2H), 3.83-3.60 (m, 7H), 3.53-3.42 (m, 1H), 3.06- 3.03 (m, 4H), 2.35-2.20 (m, 1H), 2.20-2.09 (m, 1H). LC-MS [M + H]⁺ 501.2235 |
| 624 | 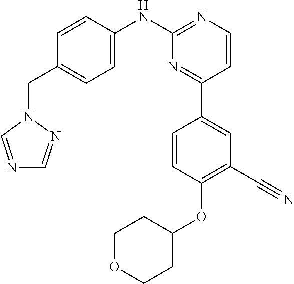 [SMILES: N#Cc1cc(-c2ccnc(Nc3ccc(Cn4cncn4)cc3)n2)ccc1OC1CCOCC1] | 2-(Tetrahydro-2H- pyran-4-yloxy)-5-(2- {[4-(1H-1,2,4- triazol-1- ylmethyl)phenyl] amino}pyrimidin-4- yl)benzonitrile | ¹H NMR (DMSO-d₆) δ 9.76 (s, 1H), 8.64 (s, 1H), 8.55-8.53 (m, 2H), 8.44 (dd, 1H), 7.98 (s, 1H), 7.7 (d, 2H), 7.55 (d, 1H), 7.48 (d, 1H), 7.26 (d, 2H), 5.35 (s, 2H), 4.97- 4.92 (m, 1H), 3.90-3.85 (m, 2H), 3.58-3.53 (m, 2H), 2.07-2.02 (m, 2H), 1.73-1.67 (m, 2H). LC-MS [M + H]⁺ 454.1996 |
| 625 | 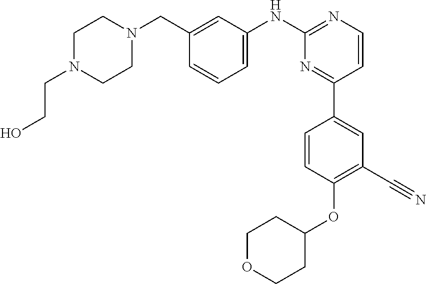 [SMILES: N#Cc1cc(-c2ccnc(Nc3cccc(CN4CCN(CCO)CC4)c3)n2)ccc1OC1CCOCC1] | 5-{2-[(3-{[4-(2- Hydroxyethyl) piperazin-1- yl]methyl}phenyl) amino]pyrimidin- 4-yl}- 2-(tetrahydro-2H- pyran-4- yloxy)benzonitrile | ¹H NMR (MeOH-d₄) δ 8.53 (d, 1H), 8.47 (d, 1H), 8.39 (dd, 1H), 7.99 (s, 1H), 7.65 (d, 1H), 7.41-6.35 (m, 3H), 7.12 (d, 1H), 4.98-4.90 (m, 1H), 4.02-3.97 (m, 4H), 3.87-3.84 (m, 2H), 3.69-3.64 (m, 2H), 3.48- 3.42 (m, 4H), 3.25-3.14 (m, 6H), 2.16-2.07 (m, 2H), 1.88-1.79 (m, 2H). LC-MS [M + H]⁺ 515.2701 |
| 626 | 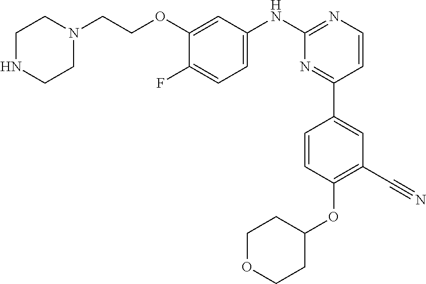 [SMILES: N#Cc1cc(-c2ccnc(Nc3ccc(F)c(OCCN4CCNCC4)c3)n2)ccc1OC1CCOCC1] | 5-[2-({4-Fluoro-3-[2- (piperazin-1- yl)ethoxy]phenyl} amino)pyrimidin- 4-yl]- 2-(tetrahydro-2H- pyran-4- yloxy)benzonitrile | ¹H NMR (DMSO-d₆) δ ppm 9.79 (s, 1H), 9.23 (s, 2H), 8.57 (d, 1H), 8.56 (s, 1H), 8.44 (dd, 1H), 7.86 (d, 1H), 7.55 (d, 1H), 7.50 (d, 1H), 7.27-7.36 (m, 1H), 7.20 (dd, 1H), 4.90-5.01 (m, 1H), 4.36-4.47 (m, 2H), 3.82-3.92 (m, 2H), 3.50- 3.60 (m, 4H), 3.39 (s, 6H), 2.00- 2.10 (m, 2H), 1.65-1.75 (m, 2H); LC-MS [M + H]⁺ 519.2514. |
| 627 | 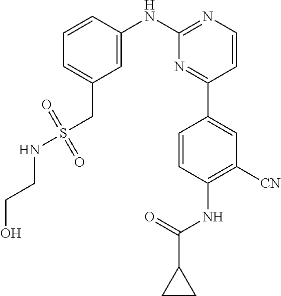 [SMILES: N#Cc1cc(-c2ccnc(Nc3cccc(CS(=O)(=O)NCCO)c3)n2)ccc1NC(=O)C1CC1] | N-(2-Cyano-4-{2- [(3-{[(2- hydroxyethyl) sulfamoyl]methyl} phenyl) amino]pyrimidin-4- yl}phenyl) cyclopropane- carboxamide | ¹H NMR (CDCl₃) δ 8.45 (d, 1H), 8.39-8.37 (m, 2H), 8.29-8.27 (m, 1H), 7.87 (s, 1H), 7.64 (d, 1H), 7.40-7.35 (m, 1H), 7.16-7.10 (m, 2H), 4.33 (s, 2H), 3.65-3.62 (m, 2H), 3.19-3.17 (m, 2H), 1.83-1.79 (m, 1H), 1.16-1.14 (m, 2H), 1.00- 0.98 (m, 2H). LC-MS [M + H]⁺ 493.1665 |
| 628 | 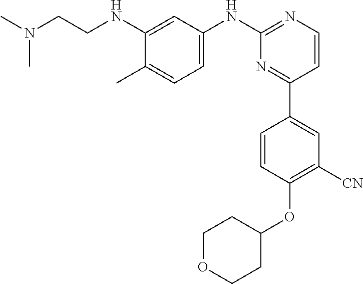 [SMILES: Cc1ccc(Nc2nccc(-c3ccc(OC4CCOCC4)c(C#N)c3)n2)cc1NCCN(C)C] | 5-[2-[[3-(2- dimethylamino- ethylamino)- 4-methyl- phenyl]amino} pyrimidin-4-yl]-2- tetrahydropyran-4- yloxy-benzonitrile | ¹H NMR (CDCl₃) δ 8.45 (d, 1H), 8.36 (s, 1H), 8.24 (d, 1H), 7.20 (s, 1H), 7.07-7.01 (m, 4H), 6.85 (d, 1H), 4.74-4.73 (m, 1H), 4.35 (s, 1H), 4.07-4.01 (m, 2H), 3.69-3.63 (m, 2H), 3.25-3.23 (m, 2H), 2.66- 2.63 (m, 2H), 2.27 (s, 6H), 2.15 (s, 3H), 2.11-2.05 (m, 2H), 1.97-1.89 (m, 2H). LC-MS [M + H]⁺ 473.2658 |
| 629 | 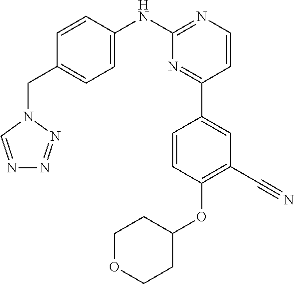 [SMILES: N#Cc1cc(-c2ccnc(Nc3ccc(Cn4cnnn4)cc3)n2)ccc1OC1CCOCC1] | 2-(Tetrahydro-2H- pyran-4-yloxy)-5-(2- {[4-(1H-tetrazol-1- ylmethyl)phenyl] amino}pyrimidin-4- yl)benzonitrile | ¹H NMR (DMSO-d₆) δ 9.82 (s, 1H), 9.51 (s, 1H), 8.56 (d, 1H), 8.54 (d, 1H), 8.44 (dd, 1H), 7.82 (d, 2H), 7.55 (d, 1H), 7.50 (d, 1H), 7.33 (d, 2H), 5.65 (s, 2H), 4.98-4.91 (m, 1H), 3.90-3.85 (m, 2H), 3.58-3.53 (m, 2H), 2.07-2.02 (m, 2H), 1.73- 1.65 (m, 2H). LC-MS [M + H]⁺ 455.1948 |
| 630 | 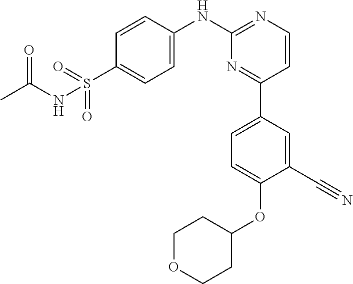 [SMILES: CC(=O)NS(=O)(=O)c1ccc(Nc2nccc(-c3ccc(OC4CCOCC4)c(C#N)c3)n2)cc1] | N-{[4-{4-[3-Cyano- 4-(tetrahydro-2H- pyran-4- ylopxy)phenyl] pyrimidin-2- yl}amino)phenyl] sulfonyl}acetamide | ¹H NMR (CDCl₃) δ 11.95 (s, 1H), 10.29 (s, 1H), 8.65 (d, 1H), 8.59 (d, 1H), 8.52-8.49 (m, 1H), 8.05-8.03 (m, 2H), 7.87-7.84 (m, 2H), 7.64- 7.58 (m, 2H), 4.96 (m, 1H), 3.91- 3.84 (m, 2H), 3.59-3.53 (m, 2H), 2.08-2.03 (m, 2H), 1.74-1.67 (m, 2H). LC-MS [M + H]⁺ 494.1568 |
| 631 | 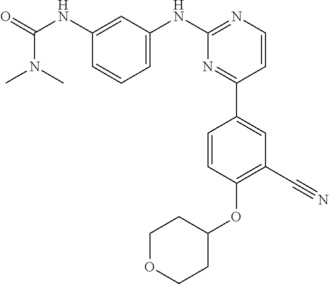 [SMILES: CN(C)C(=O)Nc1cccc(Nc2nccc(-c3ccc(OC4CCOCC4)c(C#N)c3)n2)c1] | 3-[3-({4-[3-Cyano-4- (tetrahydro-2H- pyran-4- yloxy)phenyl] pyrimidin-2- yl}amino)phenyl]- 1,1-dimethylurea | LC-MS [M + H]⁺ 459.2153 [M + Na] 481.1976 |
| 632 | 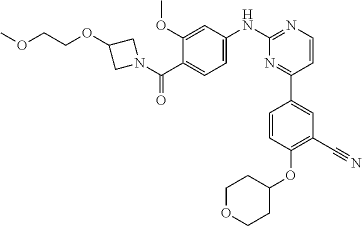 [SMILES: COCCOC1CN(C(=O)c2ccc(Nc3nccc(-c4ccc(OC5CCOCC5)c(C#N)c4)n3)cc2OC)C1] | 5-{2-[(3-Methoxy-4- {[3-(2- methoxyethoxy) azetidin-1- yl]carbonyl}phenyl) amino]pyrimidin-4- yl}-2-(tetrahydro- 2H-pyran-4- yloxy)benzonitrile | ¹H NMR (DMSO-d₆) δ 9.95 (s, 1H), 8.61 (d, 1H), 8.59 (d, 1H), 8.46 (dd, 1H), 7.85 (s, 1H), 7.57-7.54 (m, 2H), 7.33 (d, 1H), 7.26 (dd, 1H), 4.99-4.92 (m, 1H), 4.34-4.29 (m, 1H), 4.19-4.14 (m, 1H), 4.08-4.04 (m, 1H), 3.89-3.83 (m, 2H), 3.88 (s, 3H), 3.79-3.74 (m, 2H), 3.59-3.53 (m, 2H), 3.50-3.47 (m, 2H), 3.44- 3.42 (m, 2H), 3.24 (s, 3H), 2.09- 2.02 (m, 2H), 1.73-1.65 (m, 2H). LC-MS [M + H]⁺ 560.2501 |
| 633 | 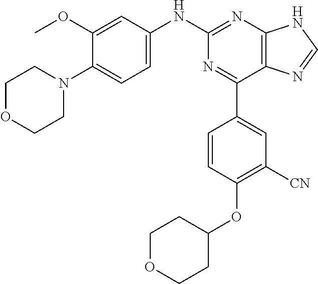 [SMILES: COc1cc(Nc2nc(-c3ccc(OC4CCOCC4)c(C#N)c3)c3nc[nH]c3n2)ccc1N1CCOCC1] | 5-(2-{[3-Methoxy-4- (morpholin-4- yl)phenyl]amino}- 3H-purin-6-yl)-2- (tetrahydro-2H- pyran-4- yloxy)benzonitrile | ¹H NMR (DMSO) δ 9.63 (bs, 1H), 9.13-9.09 (m, 2H), 8.37 (s, 1H), 7.86 (bs, 1H), 7.62 (d, 1H), 7.37 (d, 1H), 7.81 (bs, 1H), 5.00-4.94 (m, 1H), 3.91-3.87 (m, 9H), 3.60-3.54 (m, 2H), 3.24 (bs, 4H), 2.09-2.06 (m, 2H), 1.76-1.69 (m, 2H). LC-MS [M + H]⁺ 528.2352. |
| 634 | 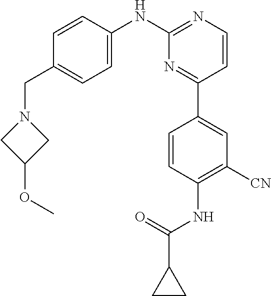 [SMILES: COC1CN(Cc2ccc(Nc3nccc(-c4ccc(NC(=O)C5CC5)c(C#N)c4)n3)cc2)C1] | N-{2-Cyano-4-[2- ({4-[(3- methoxyazetidin-1- yl)methyl]phenyl} amino)pyrimidin-4- yl]phenyl} cyclopropane- carboxamide | ¹H NMR (CDCl₃) δ 8.58 (d, 1H), 8.48 (d, 1H), 8.34 (s, 1H), 8.23 (d, 1H), 8.04 (s, 1H), 7.61 (d, 2H), 7.36-7.27 (m, 3H), 7.08 (d, 1H), 4.09-4.05 (m, 1H), 3.69-3.64 (m, 4H), 3.26 (s, 3H), 3.00-2.96 (m, 2H), 1.69-1.65 (m, 1H), 1.18-1.16 (m, 2H), 1.01-0.98 (m, 2H). LC- MS [M + H]⁺ 455.2188 |
| 635 | 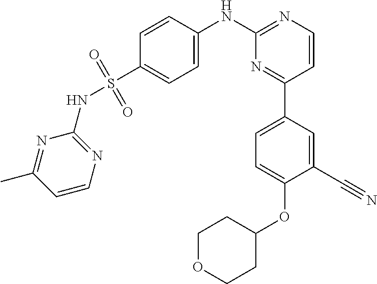 [SMILES: Cc1ccnc(NS(=O)(=O)c2ccc(Nc3nccc(-c4ccc(OC5CCOCC5)c(C#N)c4)n3)cc2)n1] | 4-({4-[3-Cyano-4- (tetrahydro-2H- pyran-4- yloxy)phenyl] pyrimidin-2-yl} amino)-N- (4-methylpyrimidin- 2- yl)benzene- sulfonamide | ¹H NMR (CDCl₃) δ 11.49 (s, 1H), 10.2 (s, 1H), 8.63-8.33 (m, 4H), 7.96 (m, 3H), 7.59 (m, 2H), 6.91 (m, 1H), 4.95 (m, 1H), 3.88 (m, 2H), 3.57 (m, 2H), 3.34 (m, 3H), 2.05 (m, 2H), 1.70 (m, 2H). LC- MS [M + H]⁺ 544.1768 |
| 636 | 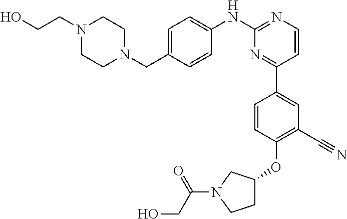 [SMILES: N#Cc1cc(-c2ccnc(Nc3ccc(CN4CCN(CCO)CC4)cc3)n2)ccc1O[C@@H]1CCN(C(=O)CO)C1] | 2-{[(3R)-1- (Hydroxyacetyl) pyrrolidin-3-yl] oxy}-5- {2-[(4-{[4-(2- hydroxyethyl) piperazin-1- yl]methyl}phenyl) amino]pyrimidin-4- yl}benzonitrile | ¹H NMR (DMSO-d₆) δ ppm 9.69 (s, 1JH), 8.53-8.57 (m, 2H), 8.49 (dd, 1H), 7.74 (d, 2H), 7.51-7.55 (m, 1H), 7.47 (d, 1H), 7.33 (d, 2H), 5.30-5.45 (m, 1H), 4.66-4.71 (m, 1H), 4.35-4.40 (m, 1H), 4.05- 4.08 (m, 1H), 3.99-4.03 (m, 1H), 3.77-3.86 (m, 1H), 3.61-3.72 (m, 3H), 3.42-3.52 (m, 4H), 3.37- 3.40 (m, 2H), 2.09-2.49 (m, 10 H); LC-MS [M + H]⁺ 558.2823. |
| 637 | 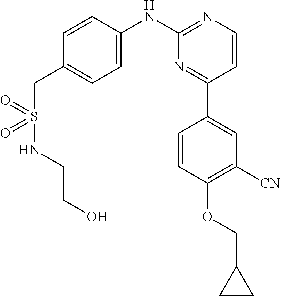 [SMILES: N#Cc1cc(-c2ccnc(Nc3ccc(CS(=O)(=O)NCCO)cc3)n2)ccc1OCC1CC1] | 1-[4-({4-[3-Cyano-4- (cyclopropylmethoxy) phenyl]pyrimidin-2- yl}amino)phenyl]-N- (2- hydroxyethyl) methanesulfonamide | ¹H NMR (CDCl₃) δ 8.44 (d, 1H), 8.30-8.24 (m, 2H), 7.71 (d, 2H), 7.41 (d, 2H), 7.12-7.08 (m, 2H), 4.29 (s, 2H), 4.04 (d, 2H), 3.64- 3.61 (m, 2H), 3.13-3.10 (m, 2H), 1.39-1.31 (m, 1H), 0.74-0.69 (m, 2H), 0.46-0.42 (m, 2H). LC-MS [M + H]⁺ 480.1696 |
| 638 | 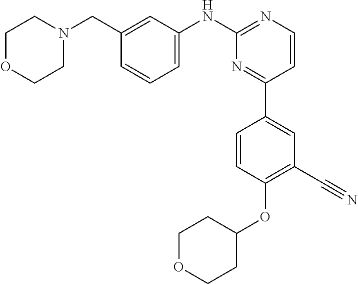 [SMILES: N#Cc1cc(-c2ccnc(Nc3cccc(CN4CCOCC4)c3)n2)ccc1OC1CCOCC1] | 5-(2-{[3-(Morpholin- 4- ylmethyl)phenyl] amino}pyrimidin- 4-yl)- 2-(tetrahydro-2H- pyran-4- yloxy)benzonitrile | ¹H NMR (CDCl₃) δ 8.40 (d, 1H), 8.30-8.26 (m, 2H), 8.01 (s, 1H), 7.56 (d, 1H), 7.50 (t, 1H), 7.28- 7.19 (m, 3H), 4.86-4.81 (m, 1H), 4.28 (s, 2H), 4.06-3.94 (m, 6H), 3.7103.65 (m, 2H), 2.98-2.88 (m, 2H), 2.15-2.08 (m, 2H), 1.98-1.90 (m, 2H). LC-MS [M + H]⁺ 472.2315 |
| 639 | 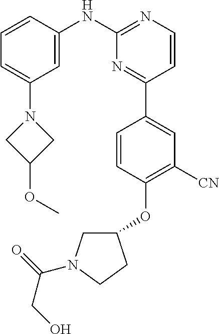 [SMILES: COC1CN(c2cccc(Nc3nccc(-c4ccc(O[C@@H]5CCN(C(=O)CO)C5)c(C#N)c4)n3)c2)C1] | 2-{[(3R)-1- (Hydroxyacetyl) pyrrolidin-3- yloxy]-5- (2-{[3-(3- methoxyazetidin-1- yl)phenyl]amino} pyrimidin-4- yl)benzonitrile | ¹H NMR (CDCl₃) δ 8.46 (d, 1H), 8.39 (s, 1H), 8.26 (d, 1H), 7.26- 7.14 (m, 2H), 7.07-7.00 (m, 3), 6.90 (d, 1H), 6.221 (d, 1H), 5.21- 5.15 (m, 1H), 4.41-4.38 (m, 1H), 4.22-4.06 (m, 4H), 4.00-3.92 (m, 1H), 3.84-3.56 (m, 5H), 3.36 (s, 3H), 2.50-2.24 (m, 2H). LC-MS [M + H]⁺ 501.2261 |
| 640 | 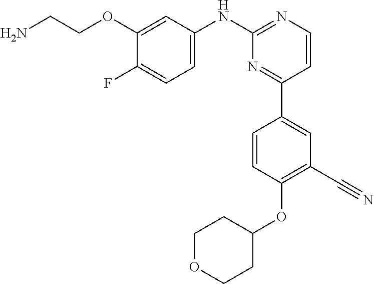 [SMILES: N#Cc1cc(-c2ccnc(Nc3ccc(F)c(OCCN)c3)n2)ccc1OC1CCOCC1] | 5-(2-{[3-(2- Aminoethoxy)-4- fluorophenyl] amino} pyrimidin-4-yl)-2- (tetrahydro-2H- pyran-4- yloxy)benzonitrile | ¹H NMR (DMSO-d₆) δ ppm 9.78 (s, 1H), 8.55-8.59 (m, 2H), 8.43 (dd, 1H), 8.03 (br. s., 3H), 7.82-7.90 (m, 1H), 7.56 (d, 1H), 7.50 (d, 1H), 7.29-7.36 (m, 1H), 7.16-7.24 (m, 1H), 4.90-5.01 (m, 1H), 4.29 (t, 2H), 3.82-3.92 (m, 2H), 3.56 (ddd, 2H), 3.27-3.36 (m, 2H), 1.99- 2.09 (m, 2H), 1.64-1.75 (m, 2H); LC-MS [M + H]⁺ 450.1937 |
| 641 | 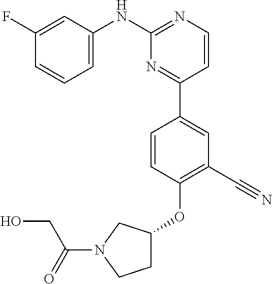 [SMILES: N#Cc1cc(-c2ccnc(Nc3cccc(F)c3)n2)ccc1O[C@@H]1CCN(C(=O)CO)C1] | 5-{2-[(3- Fluorophenyl)amino] pyrimidin-4-yl}-2- {[(3R)-1- (hydroxyacetyl) pyrrolidin-3- yl]oxy}benzonitrile | ¹H NMR (DMSO-d6) δ 9.98 (s, 1H), 8.61 (d, 1H), 8.55 (d, 1H), 8.49-8.46 (m, 1H), 7.89-7.85 (m, 1H), 7.55-7.53 (m, 1H), 7.37-7.31 (m, 1H), 6.81-6.76 (m, 1H), 5.44- 5.35 (m, 1H), 4.75-4.70 (m, 1H), 4.10-4.00 (m,, 2H), 3.85-3.54 (m, 2H), 2.52-2.14 (m, 2H). LC-MS [M + H]⁺ 434.1729 |
| 642 | 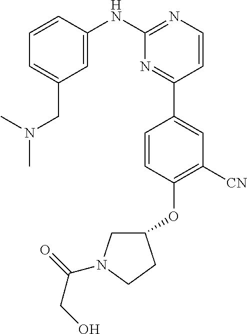 [SMILES: CN(C)Cc1cccc(Nc2nccc(-c3ccc(O[C@@H]4CCN(C(=O)CO)C4)c(C#N)c3)n2)c1] | 5-[2-({3- [(Dimethylamino) methyl]phenyl} amino) pyrimidin-4-yl]-2- {[(3R)-1- (hydroxyacetyl) pyrrolidin-3- yl]oxy}benzonitrile | ¹H NMR (MeOH-d₄) δ 8.45-8.43 (m, 2H), 8.37 (d, 1H), 7.77 (s, 1H), 7.62 (d, 1H), 7.35-7.25 (m, 3), 6.98 (d, 1H), 5.35-5.30 (m, 1H), 4.23- 4.12 (m, 2H), 3.89-3.62 (m, 4H), 3.49 (s, 2H), 2.37-2.24 (m, 2H), 2.28 (s, 6H). LC-MS [M + H]⁺ 473.2272 |
| 643 | 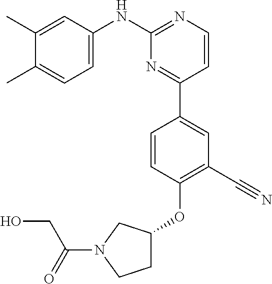 [SMILES: Cc1ccc(Nc2nccc(-c3ccc(O[C@@H]4CCN(C(=O)CO)C4)c(C#N)c3)n2)cc1C] | 5-{2-[(3,4- Dimethylphenyl) amino]pyrimidin- 4-yl}- 2-{[(3R)-1- (hydroxyacetyl) pyrrolidin-3- yl]oxy}benzonitrile | ¹H NMR (DMSO-d6) δ 9.54 (s, 1H), 8.55-8.52 (m, 2H), 8.48-8.45 (m, 1H), 7.62 (s, 1H), 7.53-7.43 (m, 3H), 7.06 (d, 1H), 5.44-5.32 (m, 1H), 4.08-4.00 (m, 2H), 3.79-3.60 (m, 2H), 3.53-3.31 (m, 2H), 2.30- 2.13 (m, 2H), 2.23 (s, 3H), 2.18 (s, 3H). LC-MS [M + H]⁺ 444.2076 |
| 644 | 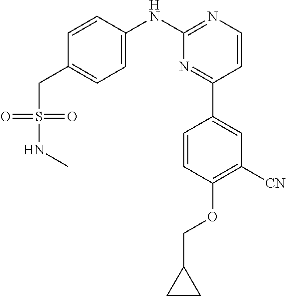 [SMILES: CNS(=O)(=O)Cc1ccc(Nc2nccc(-c3ccc(OCC4CC4)c(C#N)c3)n2)cc1] | 1-[4-({4-[3-Cyano-4- (cyclopropylmethoxy) phenyl]pyrimidin-2- yl}amino)phenyl]-N- methylmethane- sulfonamide | ¹H NMR (CDCl₃) δ 8.45 (bs, 1H), 8.28-8.23 (m, 2H), 7.71 (d, 1H), 7.39 (d, 2H), 7.09-7.07 (m, 2H), 4.25 (s, 2H), 4.03 (d, 2H), 2.73 (s, 3H), 1.41-1.34 (m, 1H), 0.78-0.69 (m, 2H), 0.49-0.42 (m, 2H). LC- MS [M + H]⁺ 450.1559 |
| 645 | 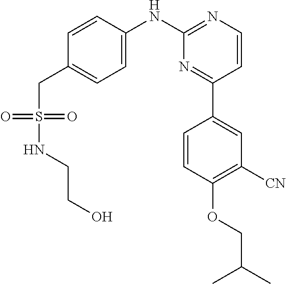 [SMILES: CC(C)COc1ccc(-c2ccnc(Nc3ccc(CS(=O)(=O)NCCO)cc3)n2)cc1C#N] | 1-[4-({4-[3-Cyano-4- (2- methylpropoxy) phenyl]pyrimidin-2- yl}amino)phenyl]-N- (2- hydroxyethyl) methanesulfonamide | ¹H NMR (CDCl₃) δ 8.43 (d, 1H), 8.30-8.25 (m, 2H), 7.72 (d, 2H), 7.40 (d, 2H), 7.13-7.11 (m, 2H), 4.29 (s, 2H), 3.94 (d, 2H), 3.63- 3.60 (m, 2H), 3.12-3.10 (m, 2H), 2.24-2.18 (m, 1H), 1.10 (d, 6H). LC-MS [M + H]⁺ 482.1773 |
| 646 | 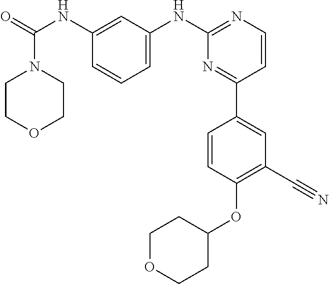 [SMILES: N#Cc1cc(-c2ccnc(Nc3cccc(NC(=O)N4CCOCC4)c3)n2)ccc1OC1CCOCC1] | N-[3-({4-[3-Cyano- 4-(tetrahydro-2H- pyran-4- yloxy)phenyl] pyrimidin-2- yl}amino)phenyl] morpholine-4- carboxamide | ¹H NMR (DMSO-d₆) δ 9.64 (s, 1H), 8.61-8.60 (m, 2H), 8.55-8.52 (m, 2H), 8.20 (s, 1H), 7.52-7.46 (m, 2H), 7.24-7.12 (m, 2H), 7.01 (d, 1H), 4.99-4.91 (m, 1H), 3.90-3.85 (m, 2H), 3.63-3.53 (m, 2H), 3.47- 3.44 (m, 4H), 2.07-2.03 (m, 2H), 1.73-1.65 (m, 2H). LC-MS [M + H]⁺ 501.2339 |
| 647 | 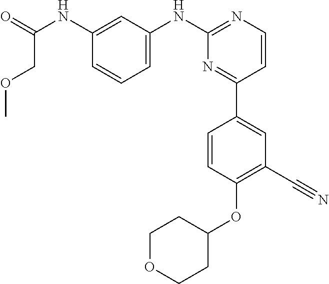 [SMILES: COCC(=O)Nc1cccc(Nc2nccc(-c3ccc(OC4CCOCC4)c(C#N)c3)n2)c1] | N-[3-({4-[3-Cyano- 4-(tetrahydro-2H- pyran-4- yloxy)phenyl] pyrimidin-2- yl}amino)phenyl]-2- methoxyacetamide | ¹H NMR (DMSO-d₆) δ 9.72-9.71 (m, 2H), 8.61 (d, 1H), 8.54 (d, 1H), 8.49 (dd, 1H), 8.42 (br s, 1H), 7.51- 7.47 (m, 2H), 7.36 (d, 1H), 7.24- 7.17 (m, 2H), 4.98-4.90 (m, 1H), 4.04 (s, 2H), 3.90-3.85 (m, 2H), 3.59-3.53 (m, 2H), 3.39 (s, 3H), 2.06-2.00 (m, 2H), 1.73-1.65 (m, 2H). LC-MS [M + H]⁺ 460.1980 |
| 648 | 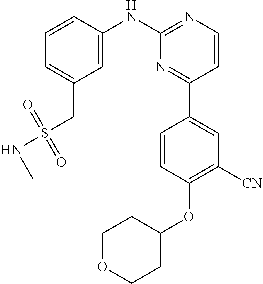 [SMILES: CNS(=O)(=O)Cc1cccc(Nc2nccc(-c3ccc(OC4CCOCC4)c(C#N)c3)n2)c1] | 1-[3-({4-[3-Cyano-4- (tetrahydro-2H- pyran-4- yloxy)phenyl] pyrimidin-2- yl}amino)phenyl]-N- methylmethane- sulfonamide | ¹H NMR (CDCl₃) δ 8.36 (d, 1H), 8.26-8.24 (m, 2H), 7.91 (s, 1H), 7.57 (d, 1H), 7.35-7.29 (m, 2H), 7.14-7.01 (m, 3H), 4.77-4.75 (m, 1H), 4.35-4.26 (m, 2H), 4.06-4.01 (m, 2H), 3.70-3.65 (m, 2H), 2.81 (s, 3H), 2.12-2.07 (m, 2H), 1.91-1.89 (m, 2H). LC-MS [M + H]⁺ 480.1699 |
| 649 | 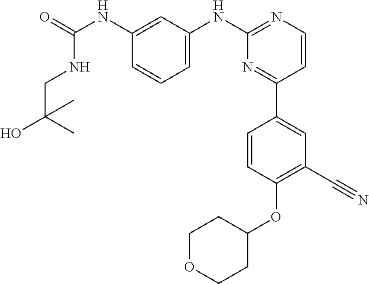 [SMILES: CC(C)(O)CNC(=O)Nc1cccc(Nc2nccc(-c3ccc(OC4CCOCC4)c(C#N)c3)n2)c1] | 1-[3-({4-[3-Cyano-4- (tetrahydro-2H- pyran-4- yloxy)phenyl] pyrimidin-2- yl}amino)phenyl]-3- (2-hydroxy-2- methylpropyl)urea | ¹H NMR (MeOH-d₄) δ 8.48-8.41 (m, 3H), 7.96 (br s, 1H), 7.57 (br s, 1H), 7.34-7.24 (m, 4H), 6.99 (d, 2H), 4.92-4.84 (m, 1H), 4.60-4.00 (m, 2H), 3.73-3.68 (m, 2H), 3.25 (s, 2H), 2.16-2.08 (m, 2H), 1.97-1.89 (m, 2H), 1.24 (s, 6H). LC-MS [M + H]⁺ 503.2407 |
| 650 | 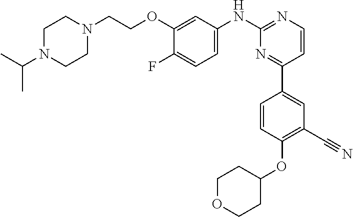 [SMILES: CC(C)N1CCN(CCOc2cc(Nc3nccc(-c4ccc(OC5CCOCC5)c(C#N)c4)n3)ccc2F)CC1] | 5-{2-[(4-Fluoro-3- {2-[4-(propan-2- yl)piperazin-1- yl]ethoxy}phenyl) amino]pyrimidin-4-yl}- 2-(tetrahydro-2H- pyran-4- yloxy)benzonitrile | ¹H NMR (DMSO-d₆) δ ppm 9.76 (s, 1H), 8.57 (d, 1H), 8.56 (s, 1H), 8.44 (dd, 1H), 7.82-7.92 (m, 1H), 7.55 (d, 1H), 7.50 (d, 1H), 7.23- 7.34 (m, 1H), 7.13-7.23 (m, 1H), 4.90-5.00 (m, 1H), 4.25-4.35 (m, 2H), 3.81-3.91 (m, 2H), 3.50- 3.60 (m, 3H), 3.47 (s, 3H), 3.40 (s, 2H), 3.20 (s, 2H), 3.10 (s, 2H), 2.81 (s, 2H), 1.97-2.10 (m, 2H), 1.63-1.74 (m, 2H), 1.24 (d, 6H); LC-MS [M + H]⁺ 561.2977. |
| 651 | 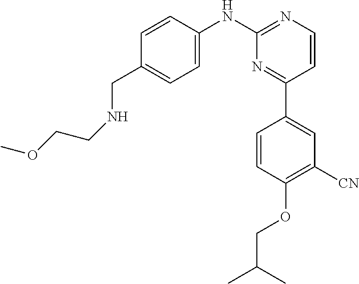 [SMILES: COCCNCc1ccc(Nc2nccc(-c3ccc(OCC(C)C)c(C#N)c3)n2)cc1] | 5-{2-[(4-{[(2- Methoxyethyl)amino] methyl}phenyl)amino] pyrimidin-4-yl}-2- (2- methylpropoxy) benzonitrile] | ¹H NMR (CDCl₃) δ 8.45 (d, 1H), 8.27-8.24 (m, 2H), 7.73 (d, 2H), 7.50 (d, 2H), 7.11-7.09 (m, 2H), 4.13 (s, 2H), 3.93 (d, 2H), 3.74- 3.72 (m, 2H), 3.39 (s, 3H), 3.09- 3.07 (m, 2H), 2.25-2.19 (m, 1H), 1.10 (d, 6H). LC-MS [M + H]⁺ 432.2406 |
| 652 | 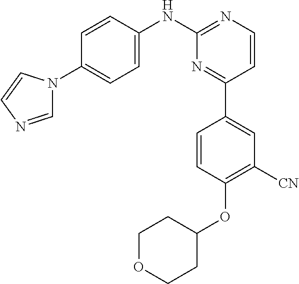 [SMILES: N#Cc1cc(-c2ccnc(Nc3ccc(-n4ccnc4)cc3)n2)ccc1OC1CCOCC1] | 5-(2-{[4-(1H- Imidazol-1- yl)phenyl]amino} pyrimidin-4-yl)-2- (tetrahydro-2H- pyran-4- yloxy)benzontrile | ¹H NMR (DMSO) δ 10.64 (s, 1H), 9.10-9.04 (m, 2H), 8.87 (m, 1H), 8.76 (dd, 1H), 8.45-8.35 (m, 2H), 7.60-7.55 (m, 3H), 6.79-6.76 (m, 2H), 5.07-5.02 (m, 1H), 3.91-3.81 (m, 2H), 3.61-3.53 (m, 2H), 2.08- 2.03 (m, 2H), 1.75-1.70 (m, 2H). LC-MS [M + H]⁺ 439.1863. |
| 653 | 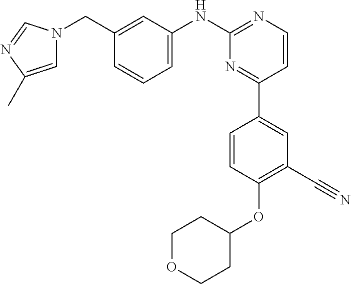 [SMILES: Cc1cn(Cc2cccc(Nc3nccc(-c4ccc(OC5CCOCC5)c(C#N)c4)n3)c2)cn1] | 5-[2-({3-[(4-Methyl- 1H-imidazol-1- yl)methyl]phenyl} amino)pyrimidin-4-yl]- 2-(tetrahydro-2H- pyran-4- yloxy)benzonitrile | ¹H NMR (CDCl₃) δ 8.36-8.22 (m, 3H), 7.77 (d, 1H), 7.51-7.47 (m, 1H), 7.25-7.17 (m, 3H), 6.88 (s, 1H), 5.25 (s, 2H), 4.84-4.76 (m, 1H), 4.06-4.00 (m, 2H), 3.70-3.64 (m, 2H), 2.36 (s, 3H), 2.13-2.08 (m, 2H), 1.97-1.89 (m, 2H). LC-MS [M + H]⁺ 467.2208 |
| 654 | 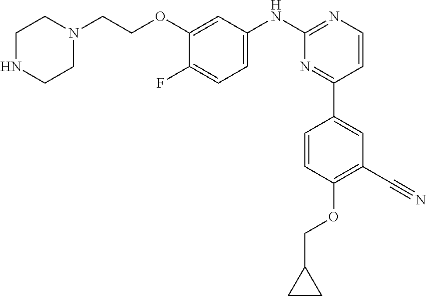 [SMILES: N#Cc1cc(-c2ccnc(Nc3ccc(F)c(OCCN4CCNCC4)c3)n2)ccc1OCC1CC1] | 2- (Cyclopropylmethoxy)- 5-[2-({4-fluoro-3- [2-(piperazin-1- yl)ethoxy]phenyl} amino)pyrimidin-4- yl]benzonitrile | ¹H NMR (DMSO-d₆) δ ppm 9.78 (s, 1H), 9.14 (s, 2H), 8.56 (d, 1H), 8.55 (s, 1H), 8.45 (dd, 1H), 7.86 (dd, 1H), 7.50 (d, 1H), 7.41 (d, 1H), 7.26-7.36 (m, 1H), 7.20 (dd, 1H), 4.34-4.44 (m, 2H), 4.12 (d, 2H), 3.48 (s, 2H), 3.34 (s, 7H), 1.26-1.36 (m, 1H), 0.59-0.70 (m, 2H), 0.39-0.47 (m, 2H); LC-MS [M + H]⁺ 489.2411. |
| 655 | 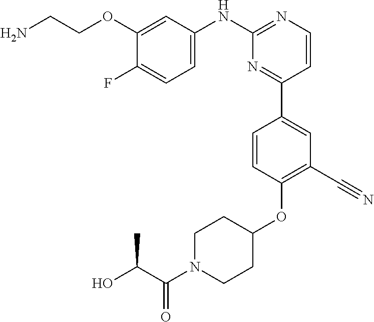 [SMILES: C[C@H](O)C(=O)N1CCC(Oc2ccc(-c3ccnc(Nc4ccc(F)c(OCCN)c4)n3)cc2C#N)CC1] | 5-(2-{[3-(2- Aminoethoxy-4- fluorophenyl]amino} pyrimidin-4-yl)-2- ({1-[(2S)-2- hydroxypropanoyl] piperidin-4- yl}oxy)benzonitrile | LC-MS [M + H]⁺ 521.2326 |
| 656 | 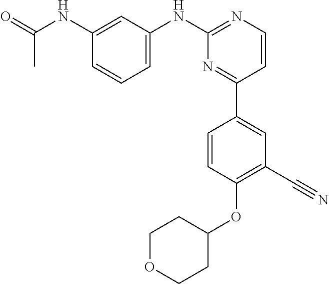 [SMILES: CC(=O)Nc1cccc(Nc2nccc(-c3ccc(OC4CCOCC4)c(C#N)c3)n2)c1] | N-[3-({4-[3-Cyano- 4-(tetrahydro-2H- pyran-4- yloxy)phenyl] pyrimidin-2- yl}amino)phenyl] acetamide | ¹H NMR (CDCl₃) δ 8.47 (d, 1H), 8.42 (s, 1H), 8.36 (d, 1H), 8.25 (dd, 1H), 7.40-7.28 (m, 3H), 7.08-7.00 (m, 3H), 4.78-4.73 (m, 1H), 4.17 (t, 2H), 4.07-4.01 (m, 2H), 3.69-3.63 (m, 2H), 2.22 (s, 3H), 2.11-2.05 (m, 2H), 1.99-1.88 (m, 2H). LC-MS [M + H]⁺ 430.1853 |
| 657 | 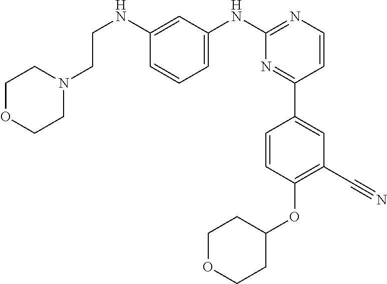 [SMILES: N#Cc1cc(-c2ccnc(Nc3cccc(NCCN4CCOCC4)c3)n2)ccc1OC1CCOCC1] | 5-{3-[(3-{[2- (Morpholin-4- yl)ethyl]amino} phenyl)amino] pyrimidin- 4-yl}2-(tetrahydro- 2H-pyran-4- yloxy)benzonitrile | ¹H NMR (CDCl₃) δ 8.39 (d, 1H), 8.28 (dd, 1H), 8.21 (d, 1H), 7.29- 7.17 (m, 3H), 7.09-7.04 (m, 2H), 6.50 (dd, 1H), 4.84-4.81 (m, 1H), 4.07-3.98 (m, 7H), 3.72-3.66 (m, 5H), 3.42 (t, 2H), 2.15-2.10 (m, 2H), 1.98-1.91 (m, 2H). LC-MS [M + H]⁺ 501.2569 |
| 658 | 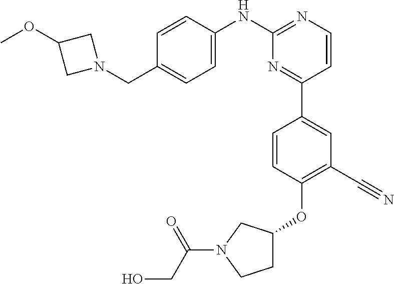 [SMILES: COC1CN(Cc2ccc(Nc3nccc(-c4ccc(O[C@@H]5CCN(C(=O)CO)C5)c(C#N)c4)n3)cc2)C1] | 2-{[(3R)-1- (Hydroxyacetyl) pyrrolidin-3-yl] oxy}-5- [2-({4-[(3- methoxyazetidin-1- yl)methyl]phenyl} amino)pyrimidin-4- yl]benzonitrile | ¹H NMR (DMSO-d₆) δ ppm 9.89- 9.94 (m, 1H), 9.80 (br. s., 1H), 8.60 (d, 1H), 8.56 (d, 1H), 8.46-8.51 (m, 1H), 7.85-7.90 (m, 2H), 7.50- 7.56 (m, 2H), 7.39-7.46 (m, 2H), 5.40-5.46 (m, 1H), 5.31-5.39 (m, 1H), 4.29-4.34 (m, 2H), 4.21- 4.28 (m, 3H), 4.07 (d, 1H), 3.91- 4.02 (m, 3H), 3.60-3.71 (m, 3H), 3.40-3.52 (m, 1H), 3.25 (d, 3H), 2.23-2.34 (m, 1H), 2.11-2.23 (m, 1H); LC-MS [M + H]⁺ 515.2417 |
| 659 | 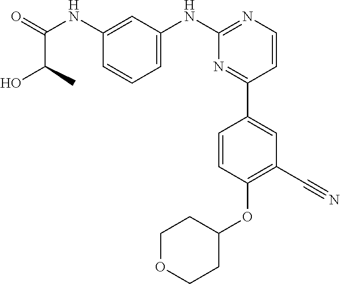 [SMILES: C[C@@H](O)C(=O)Nc1cccc(Nc2nccc(-c3ccc(OC4CCOCC4)c(C#N)c3)n2)c1] | (2R)-N-[3-({4-[3- Cyano-4-(tetrahydro- 2H-pyran-4- yloxy)phenyl] pyrimidin-2- yl}amino)phenyl]-2- hydroxypropanamide | ¹H NMR (CDCl₃) δ 8.75 (s, 1H), 8.68-8.65 (m, 2H), 8.29 (dd, 1H), 8.19 (d, 1H), 7.32 (t, 1H), 7.23- 7.18 (m, 2H), 7.10 (d, 2H), 7.17- 7.13 (m, 2H), 6.95 (s, 1H), 4.82- 4.77 (m, 1H), 4.49 (q, 1H), 4.06- 4.00 (m, 2H), 3.70-3.65 (m, 2H), 2.14-2.05 (m, 2H), 1.96-1.87 (m, 2H), 1.55 (d, 3H). LC-MS [M + H]⁺ 460.2026 |
| 660 | 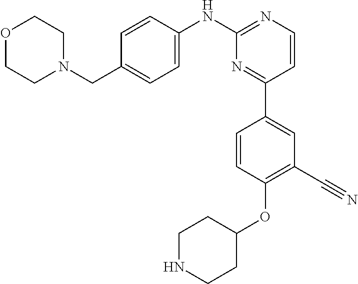 [SMILES: N#Cc1cc(-c2ccnc(Nc3ccc(CN4CCOCC4)cc3)n2)ccc1OC1CCNCC1] | 5-(2-{[4-(Morpholin- 4- ylmethyl)phenyl] amino}pyrimidin- 4-yl)- 2-(piperidin-4- yloxy)benzonitrile | [M + H]⁺ 471.3 |
| 661 | 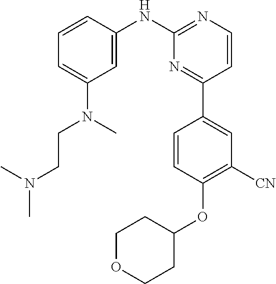 [SMILES: CN(C)CCN(C)c1cccc(Nc2nccc(-c3ccc(OC4CCOCC4)c(C#N)c3)n2)c1] | 5-[2-[[3-(2- dimethylaminoethyl) (methyl)amino) phenyl] amino]pyrimidin-4- yl]-2- tetrahydropyran-4- yloxy-benzonitrile | ¹H NMR (CDCl₃) δ 8.46 (d, 1H), 8.36 (s, 1H), 8.24 (d, 1H), 7.23- 7.09 (m, 3H), 7.08-7.04 (m, 2H), 6.92 (d, 1H), 6.46 (d, 1H), 4.77- 4.74 (m, 1H), 4.06-4.02 (m, 2H), 3.69-3.65 (m, 2H), 3.52-3.48 (m, 2H), 3.02 (s, 3H), 2.54-2.50 (m, 2H), 2.29 (s, 6H), 2.10-2.07 (m, 2H), 1.95-1.93 (m, 2H). LC-MS [M + H]⁺ 473.2664 |
| 662 | 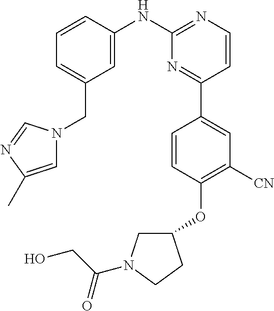 [SMILES: Cc1cn(Cc2cccc(Nc3nccc(-c4ccc(O[C@@H]5CCN(C(=O)CO)C5)c(C#N)c4)n3)c2)cn1] | 2-{[(3R)-1- (Hydroxyacetyl) pyrrolidin-3-yl] oxy}-5- [2-({3-[(4-methyl- 1H-imidazol-1- yl)methyl]phenyl} amino)pyrimidin-4- yl]benzonitrile | ¹H NMR (CDCl₃) δ 8.46 (d, 1H), 8.31 (d, 1H), 8.30-8.29 (m, 1H), 7.63-7.60 (m, 1H), 7.55-7.47 (m, 1H), 7.38-7.34 (m, 1H), 7.19-7.11 (m, 2H), 6.89-6.76 (m, 1H), 5.29- 5.23 (m, 1H), 5.10 (d, 2H), 4.21- 4.15 (m, 2H), 3.96-3.89 (m, 1H), 3.83-3.79 (m, 1H), 3.75-3.61 (m, 4H), 2.50-2.34 (m, 2H), 2.18-2.14 (m, 3H). LC-MS [M + H]⁺ 510.2220 |
| 663 | 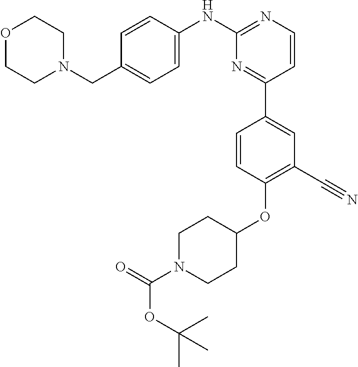 [SMILES: CC(C)(C)OC(=O)N1CCC(Oc2ccc(-c3ccnc(Nc4ccc(CN5CCOCC5)cc4)n3)cc2C#N)CC1] | tert-Butyl 4-[2- cyano-4-(2-{[4- (morpholin-4- ylmethyl)phenyl] amino}pyrimidin-4- yl)phenoxy] piperidine-1- carboxylate | [M + H]⁺ 571.40 |
| 664 | 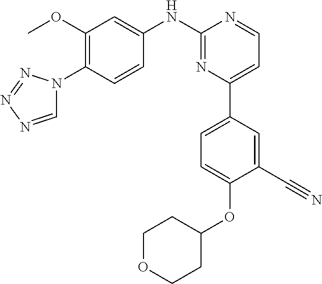 [SMILES: COc1cc(Nc2nccc(-c3ccc(OC4CCOCC4)c(C#N)c3)n2)ccc1-n1cnnn1] | 5-(2-{[3-Methoxy-4- (1H-tetrazol-1- yl)phenyl]amino} pyrimidin-4-yl)-2- (tetrahydro-2H- pyran-4- yloxy)benzonitrile | ¹H NMR (DMSO) δ 10.15 (s, 1H), 9.75 (s, 2H), 8.65 (d, 1H), 8.61 (d, 1H), 8.48 (dd, 1H), 8.12 (bs, 1H), 7.61-7.58 (m, 3H), 7.50-7.48 (m, 1H), 4.97-4.94 (m, 1H), 3.91 (s, 3H), 3.91-3.84 (m, 2H), 3.59-3.53 (m, 2H), 2.07-2.07 (m, 2H), 1.71- 1.67 (m, 2H). LC-MS [M + H]⁺ 471.1904 |
| 665 | 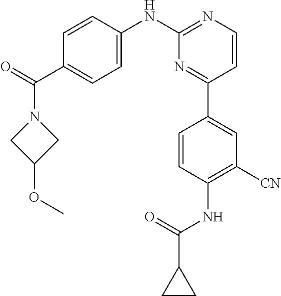 [SMILES: COC1CN(C(=O)c2ccc(Nc3nccc(-c4ccc(NC(=O)C5CC5)c(C#N)c4)n3)cc2)C1] | N-{2-Cyano-4-[2- ({4-[(3- methoxyazetidin-1- yl)carbonyl]phenyl} amino)pyrimidin-4- yl]phenyl} cyclopropane- carboxamide | ¹H NMR (CDCl₃) δ 8.52-8.46 (m, 2H), 8.33 (d, 1H), 8.26-8.23 (m, 1H), 7.77 (d, 2H), 7.67-7.64 (m, 2H), 7.17 (d, 1H), 4.53-4.36 (m, 2H), 4.28-4.22 (m, 2H), 4.09-4.03 (m, 1H), 3.33 (s, 3H), 1.81-1.77 (m, 1H), 1.18-1.14 (m, 2H), 1.01-0.98 (m, 2H). LC-MS [M + H]⁺ 469.1942 |
| 666 | 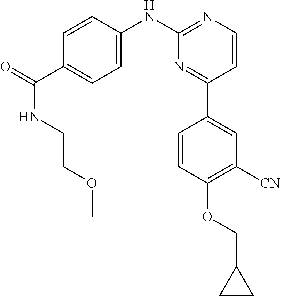 [SMILES: COCCNC(=O)c1ccc(Nc2nccc(-c3ccc(OCC4CC4)c(C#N)c3)n2)cc1] | 4-({4-[3-Cyano-4- cyclopropylmethoxy) phenyl]pyrimidin-2- yl}amino)-N-(2- methoxyethyl) benzamide | ¹H NMR (CDCl₃) δ 8.49 (d, 1H), 8.33-8.28 (m, 2H), 7.87-7.77 (m, 4H), 7.18-7.11 (m, 2H), 4.05 (d, 2H), 3.66-3.59 (m, 4H), 3.42 (s, 3H), 1.39-1.31 (m, 1H), 0.74-0.69 (m, 2H), 0.47-0.43 (m, 2H). LC- MS [M + H]⁺ 444.2026 |
| 667 | 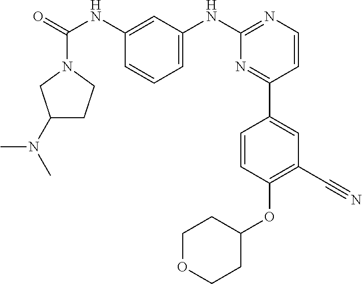 [SMILES: CN(C)C1CCN(C(=O)Nc2cccc(Nc3nccc(-c4ccc(OC5CCOCC5)c(C#N)c4)n3)c2)C1] | N-[3-({4-[3-Cyano- 4-(tetrahydro-2H- pyran-4- yloxy)phenyl] pyrimidin-2- yl}amino)phenyl]-3- (dimethylamino) pyrrolidine-1- carboxamide | ¹H NMR (MeOH-d₄) δ 8.66 (s, 1H), 8.43-8.34 (m, 3H), 7.38-7.36 (m, 2H), 7.26 (t, 1H), 7.14 (d, 1H), 7.06 (d, 1H), 4.96-4.89 (m, 1H), 4.10-4.05 (m, 1H), 4.00-3.96 (m, 3H), 3.86-3.80 (m, 1H), 3.69-3.60 (m, 4H), 2.98 (s, 6H), 2.59-2.52 (m, 1H), 2.30-2.22 (m, 1H), 2.15-2.09 (m, 2H), 1.87-1.79 (m, 2H). LC- MS [M + H]⁺ 528.2727 |
| 668 | 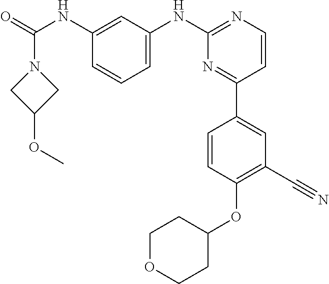 [SMILES: COC1CN(C(=O)Nc2cccc(Nc3nccc(-c4ccc(OC5CCOCC5)c(C#N)c4)n3)c2)C1] | N-[3-({4-[3-Cyano- 4-(tetrahydro-2H- pyran-4- yloxy)phenyl] pyrimidin-2- yl}amino)phenyl]-3- methoxyazetidine-1- carboxamide | ¹H NMR (DMSO-d₆) δ 9.61 (s, 1H), 8.64-8.46 (m, 4H), 8.22 (m, 1H), 7.52-7.44 (m, 2H), 7.23 (d, 1H), 7.14 (t, 1H), 7.04 (d, 1H), 4.98- 4.90 (m, 1H), 4.20-4.14 (m, 3H), 3.90-3.85 (m, 2H), 3.79-3.77 (m, 2H), 3.58-3.53 (m, 2H), 3.22 (s, 3H), 2.10-2.01 (m, 2H), 1.75-1.64 (m, 2H). LC-MS [M + H]⁺ 501.2216 |
| 669 | 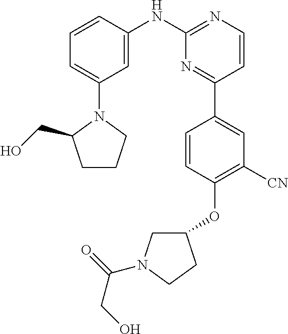 [SMILES: N#Cc1cc(-c2ccnc(Nc3cccc(N4CCC[C@H]4CO)c3)n2)ccc1O[C@@H]1CCN(C(=O)CO)C1] | 2-{[(3R)-1- (Hydroxyacetyl) pyrrolidin-3-yl] oxy}-5- [2-({3-[(2S)-2- (hydrfoxymethyl) pyrrolidin-1- yl]phenyl}amino) pyrimidin-4- yl]benzonitrile | ¹H NMR (CDCl₃) δ 8.47 (d, 1H), 8.38 (d, 1H), 8.26-8.24 (m, 1H), 7.29-7.19 (m, 2H), 7.05-7.03 (m, 2H), 6.89-6.84 (m, 1H), 6.44 (d, 1H), 5.21-5.07 (m, 1H), 4.19 (s, 2H), 4.11-3.99 (m, 1H), 3.99-3.92 (m, 2H), 3.82-3.64 (m, 4H), 3.61- 3.56 (m, 1H), 3.25-3.21 (m, 1H), 2.52-2.30 (m, 2H), 2.13-2.03 (m, 4H). LC-MS [M + H]⁺ 515.240 |
| 670 | 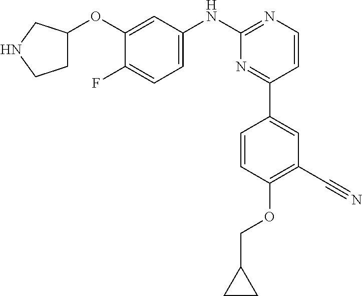 [SMILES: N#Cc1cc(-c2ccnc(Nc3ccc(F)c(OC4CCNC4)c3)n2)ccc1OCC1CC1] | 2- (Cyclopropyl- methoxy)- 5-(2-{[4-fluoro-3- (pyrrolidin-3- yloxy)phenyl]amino} pyrimidin-4- yl)benzonitrile | ¹H NMR (DMSO-d₆) δ ppm 9.74 (s, 1H), 9.19 (s, 1H), 9.10 (s, 1H), 8.56 (s, 1H), 8.54 (d, 1H), 8.41- 8.48 (m, 1H), 7.75 (dd, 1H), 7.49 (d, 1H), 7.35-7.45 (m, 2H), 7.22 (dd, 1H), 5.14 (s, 1H), 4.11 (d, 2H), 3.44-3.54 (m, 2H), 3.29-3.40 (m, 2H), 2.19-2.27 (m, 2H), 1.26- 1.36 (m, 1H), 0.60-0.68 (m, 2H), 0.37-0.43 (m, 2 H), LC-MS [M + H]⁺ 446.2006. |

The HPLC conditions used to characterize each compound listed in Table 2 are as follows:

Flow: 1.2 mL/minute

Solvents: A: H₂O+0.01% TFA
B: ACN+0.01% TFA

Gradient: 5% B for 1 minute
5% B to 100% B in 9 minutes
at 100% B for 2.4 minutes
to 0% B in 0.1 minutes
at 0% for 0.5 minutes

Overall time: 13.00 minutes

Column: XTerra MS C₁₈ 3.5 um 4.6×150 mm.

### Biochemical and Biological Examples

### In-Vitro IKKε and TBK1 Kinase Assays

IKKε enzyme was produced as a His-tag fusion in Sf9 cells and used at a final concentration of 0.04 μg/ml. TBK1 enzyme was produced as a His-tag fusion in Sf9 cells and used at a final concentration of 0.1 μg/ml. Kinase reactions were carried out in reaction buffer using myelin basic protein (Millipore, Ballerica, Mass.) or casein (Sigma, St. Louis, Mo.) as substrate at an ATP concentration equal to twice the K_{m,ATP} value for each enzyme, corresponding to 32 μM ATP for IKKε and 60 μM ATP for TBK1, in the presence of 0.3 μCi [γ³³]ATP (PerkinElmer, Waltham, Mass.). Final enzyme concentrations were 0.1 or 0.015 μg/ml (IKKε) and 0.1 or 0.02 μg/ml (TBK1), representing “normal” and “sensitized” assay conditions respectively. Test compounds (or DMSO solvent as a control) were added prior to initiation of the reactions. Reactions were terminated after 30-45 minutes by adding 3% phosphoric acid. Terminated reactions were transferred to P-81 cellulose phosphate filterplates (Whatman, Inc., Piscataway, N.J.) and washed with 1% phosphoric acid on a vacuum apparatus. After air drying, scintillant (PerkinElmer, Waltham, Mass.) was added and the plates were read on a PerkinElmer TopCount NXT instrument. Counts were normalized to DMSO controls after background subtraction.

Using the assays described above for inhibition of IKKε kinase activity, Example Compounds 7, 8, 9, 10, 36, 37, 40, 42, 44, 45, 46, 52, 53, 55, 61, 66, 69, 74, 77, 81, 84, 95, 97, 101, 108, 125, 131, 137, 142, 145, 147, 151, 153, 160, 163, 166, 180, 183, 189, 198, 204, 213, 227, 232, 234, 240, 244, 245, 249, 250, 255, 260, 265, 274, 276, 277, 282, 286, 289, 291, 292, 300, 304, 306, 308, 309, 319, 320, 322, 325, 338, 347, 348, 351, 357, 360, 365, 379, 382, 386, 388, 389, 390, 391, 398, 424, 435, 448, 451, 452, 459, 472, 474, 513, 514, and 562 were found to inhibit the kinase activity of IKKε with an IC₅₀ value ranging from about 500 nM to about 50 nM;

Example Compounds 1, 12, 13, 17, 19, 23, 38, 39, 47, 48, 49, 50, 54, 56, 57, 58, 60, 63, 64, 65, 67, 70, 71, 79, 85, 86, 87, 90, 92, 94, 99, 102, 105, 106, 110, 113, 116, 117, 120, 123, 136, 138, 139, 140, 143, 146, 149, 152, 156, 161, 167, 168, 169, 172, 173, 174, 177, 179, 182, 185, 186, 187, 188, 192, 194, 195, 196, 197, 199, 200, 201, 202, 205, 209, 214, 215, 217, 218, 219, 220, 224, 226, 229, 230, 233, 241, 243, 247, 248, 251, 254, 257, 259, 266, 267, 268, 269, 272, 273, 278, 279, 280, 281, 284, 285, 288, 294, 295, 296, 297, 299, 301, 302, 303, 305, 310, 313, 314, 315, 316, 318, 321, 323, 324, 327, 332, 333, 336, 337, 339, 342, 343, 344, 346, 352, 353, 356, 358, 359, 361, 362, 363, 364, 366, 368, 369, 372, 375, 378, 380, 383, 384, 387, 399, 407, 408, 409, 410, 411, 412, 414, 416, 417, 418, 419, 420, 421, 422, 423, 425, 426, 427, 428, 429, 430, 431, 432, 433, 434, 441, 443, 445, 447, 449, 450, 453, 454, 455, 456, 457, 460, 461, 462, 463, 464, 466, 468, 469, 470, 483, 491, 499, 508, 509, 528, 532, 537, 553, 554, 556, 557, 568, 569, 570, 582, 600, 602, 605, 623, 633, 634, and 641 were found to inhibit the kinase activity of IKKε with an IC₅₀ value ranging from about 50 nM to about 5 nM; and

Example Compounds 2, 3, 4, 5, 6, 11, 14, 15, 16, 18, 20, 21, 22, 24, 25, 26, 27, 28, 29, 30, 31, 32, 33, 34, 35, 59, 68, 72, 73, 75, 76, 80, 82, 83, 88, 91, 93, 96, 98, 100, 103, 104, 107, 111, 114, 115, 118, 124, 127, 129, 130, 132, 134, 155, 157, 158, 164, 165, 171, 176, 178, 181, 184, 190, 191, 206, 208, 210, 211, 212, 216, 223, 225, 231, 235, 237, 239, 242, 246, 253, 256, 261, 262, 264, 271, 275, 287, 290, 307, 311, 326, 329, 331, 334, 335, 341, 354, 367, 370, 371, 373, 374, 376, 377, 381, 385, 392, 393, 394, 395, 396, 397, 400, 401, 402, 403, 404, 405, 406, 413, 415, 436, 437, 438, 439, 440, 442, 444, 446, 467, 471, 475, 476, 477, 478, 479, 480, 481, 482, 484, 485, 486, 487, 488, 489, 490, 492, 493, 494, 495, 496, 497, 498, 500, 501, 502, 503, 504, 505, 506, 507, 510, 511, 512, 517, 518, 519, 520, 521, 522, 523, 524, 525, 526, 527, 529, 530, 531, 533, 534, 535, 536, 538, 539, 540, 541, 542, 543, 544, 545, 546, 547, 548, 549, 550, 552, 558, 559, 560, 561, 563, 564, 565, 566, 567, 571, 572, 573, 574, 575, 576, 577, 578, 579, 580, 581, 583, 584, 585, 586, 587, 588, 589, 590, 591, 592, 593, 594, 595, 596, 597, 598, 599, 601, 603, 604, 606, 607, 608, 609, 610, 611, 612, 613, 614, 615, 616, 617, 618, 619, 620, 621, 622, 624, 625, 626, 627, 628, 629, 630, 631, 632, 635, 636, 637, 638, 639, 640, 642, 643, 644, 645, 646, 647, 648, 649, 650, 651, 653, 654, 655, 656, 657, 658, 659, 661, 662, 664, 665, 666, 667, 668, 669, and 670 were found to inhibit the kinase activity of IKKε with an IC₅₀ value of less than about 5 nM.

Table 3, below, shows the specific IKKε kinase inhibitory activity as determined for a subset of compounds according to Formula I.

Generally, compounds found to inhibit the kinase activity of IKKε would also be expected to inhibit the kinase activity of TBK1, given the high degree of similarity of the amino acid sequences encoding these two closely-related kinases, and particularly those sequences encoding the kinase domains of these enzymes. In some cases, however, compounds found to inhibit IKKε kinase activity with an IC₅₀ of less than 100 nM, were found to inhibit TBK1 kinase activity with an IC₅₀ of greater than 5 μM. In other cases the inhibitory activity of particular compounds was found to be greater for TBK1, than for IKKε. Nevertheless, most of the compounds tested for their ability to inhibit the kinase activity of both IKKε and TBK1 were found to exhibit similar inhibitory activity against both enzymes.

Table 3, below, shows the specific TBK1 kinase inhibitory activity as determined for a subset of compounds according to Formula I.

Using the assays described above for inhibition of TBK1 kinase activity, Example Compounds 276, 389, 387, 55, 347, 286, 189, 340, 390, and 263 were found to inhibit the kinase activity of TBK1 with an IC₅₀ value ranging from about 500 nM to about 100 nM;

Example Compounds 12, 17, 45, 48, 54, 60, 63, 67, 70, 71, 72, 79, 85, 86, 90, 94, 105, 115, 117, 123, 136, 138, 149, 152, 169, 172, 177, 179, 183, 186, 201, 205, 214, 224, 226, 231, 241, 243, 248, 251, 257, 259, 260, 272, 273, 278, 280, 281, 283, 291, 294, 295, 302, 303, 305, 313, 314, 318, 320, 322, 324, 327, 332, 337, 339, 344, 346, 353, 356, 358, 359, 361, 366, 368, 372, 373, 375, 378, 380, 383, 410, 411, 412, 414, 416, 419, 420, 421, 422, 428, 432, 443, 447, 448, 457, 460, 463, 477, 484, 508, 532, 537, 553, 557, 568, 569, 570, and 634 were found to inhibit the kinase activity of TBK1 with an IC₅₀ value ranging from about 100 nM to about 10 nM; and

Example Compounds 1, 2, 3, 4, 5, 6, 11, 13, 14, 15, 16, 18, 19, 20, 21, 22, 23, 24, 25, 26, 27, 28, 29, 30, 31, 32, 33, 34, 35, 38, 49, 59, 64, 65, 68, 73, 75, 76, 80, 82, 83, 88, 91, 93, 96, 98, 100, 103, 104, 107, 110, 111, 114, 116, 118, 124, 127, 129, 130, 132, 134, 143, 155, 157, 158, 164, 165, 168, 171, 176, 178, 181, 184, 187, 190, 191, 194, 202, 206, 208, 209, 210, 211, 212, 215, 216, 217, 218, 219, 220, 223, 225, 230, 233, 235, 237, 239, 242, 246, 253, 254, 256, 261, 262, 264, 266, 268, 269, 271, 275, 284, 285, 287, 288, 290, 296, 297, 307, 311, 315, 326, 329, 331, 334, 335, 341, 342, 343, 354, 363, 367, 370, 371, 374, 376, 377, 381, 385, 392, 393, 394, 395, 396, 397, 400, 401, 402, 403, 404, 405, 406, 407, 408, 409, 413, 415, 417, 418, 423, 425, 427, 433, 434, 436, 437, 438, 439, 440, 444, 445, 446, 450, 456, 461, 466, 467, 468, 470, 471, 475, 476, 478, 479, 480, 481, 482, 483, 485, 486, 487, 488, 489, 490, 491, 492, 493, 494, 495, 496, 497, 498, 499, 500, 501, 502, 503, 504, 505, 506, 507, 509, 510, 511, 517, 518, 519, 520, 521, 522, 523, 524, 525, 526, 527, 528, 529, 530, 531, 533, 534, 536, 539, 543, 554, 556, 558, 559, 561, 565, 566, 567, 572, 574, 581, 585, 586, 588, 590, 594, 596, 597, 599, 601, 603, 606, 608, 611, 612, 613, 616, 618, 619, 620, 625, 626, 627, 631, 632, 633, 637, 640, 644, 645, 646, 648, 650, 651, 654, 657, 665, and 666 were found to inhibit the kinase activity of TBK1 with an IC₅₀ value of less than about 10 nM.

### Assays to Detect the In-Situ Phosphorylation of IRF3 (and IRF7

HEK293T cells were cotransfected in a 10-cm dish with IRF3 and IKKε expression plasmids using Lipofectamine 2000 (Invitrogen, Carlsbad, Calif.). The following day, cells were replated at 20,000 per well in 96-well plates and treated with test compounds (compounds according to Formula I) for 20 hours. Cell lysates were prepared and analyzed using an ELISA for phospho-Ser396 (anti-IRF3 capture antibody, Santa Cruz Biotechnology, Inc., Santa Cruz, Calif.; anti-p-Ser396 IRF3 detection antibody, Cell Signaling, Danvers, Mass.). pIRF3 levels in compound treated cells were normalized to DMSO treated cells (no compound). Cell viability was assayed in a parallel set of plates to monitor cytotoxic effects of the test compounds (CellTiter-Glo, Promega, Inc., Madison, Wis.). TBK1 activity was tested by Western blotting using a phospho-specific IRF7 antibody. Similar to above, HEK293T cells were transfected with IRF7 and TBK1 expression plasmids. Cells were seeded in 12-well plates at 150,000 per well and treated overnight with test compounds. Protein lysates were prepared and processed for Western blotting followed by detection using a phosphor-Ser477/Ser479 IRF7 antibody (BD Biosciences, San Jose, Calif.)

Using the assay described above, Example Compounds 3, 20, 27, 30, 35, 64, 72, 75, 103, 132, 157, 206, 208, 242, 253, 262, 290, 381, 445, 486, 528, 535, 544, 545, 577, 578, 580, 583, 601, 614, 619, 643, 655, 658, 668, and 670 were found to inhibit the in-situ IKKε-mediated phosphorylation of IRF3 with an IC₅₀ value ranging from about 500 nM to about 250 nM;

Example Compounds 18, 25, 32, 83, 93, 202, 219, 225, 256, 307, 334, 371, 377, 414, 437, 489, 494, 499, 508, 511, 524, 526, 537, 541, 547, 563, 564, 574, 586, 591, 597, 600, 603, 607, 612, 617, 640, 648, 659, and 669 were found to inhibit the in-situ IKKε-mediated phosphorylation of IRF3 with an IC₅₀ value ranging from about 250 nM to about 100 nM; and

Example Compounds 2, 5, 21, 22, 31, 59, 73, 114, 176, 178, 212, 223, 271, 354, 385, 392, 393, 395, 400, 401, 402, 404, 405, 406, 408, 413, 415, 418, 434, 436, 438, 439, 440, 442, 444, 446, 468, 471, 475, 476, 477, 478, 479, 480, 481, 482, 483, 484, 485, 487, 488, 492, 493, 495, 497, 498, 500, 501, 502, 503, 504, 505, 506, 507, 510, 512, 517, 518, 519, 520, 521, 522, 523, 525, 527, 529, 530, 531, 533, 536, 538, 540, 542, 543, 548, 552, 556, 559, 561, 567, 571, 588, 592, 593, 599, 609, 613, 616, 618, 620, 624, 625, 626, 628, 629, 631, 632, 638, 642, 646, 647, 650, 651, 653, 656, 657, 661, 662, 664, and 667 were found to inhibit the in-situ IKKε-mediated phosphorylation of IRF3 with an IC₅₀ value of less than about 100 nM.

Table 3, below, shows the specific in-situ IRF3 phosphorylation inhibitory activity of a subset of compounds according to Formula I, as determined using the assay described above.

Using the assay described above, Example Compound 5 was found to inhibit both IKKε and TBK1-mediated phosphorylation of IRF7.

TABLE 3
Activities of a Subset of Compounds According to Formula
I in Inhibiting the Kinase Activities of IKKε and
TBK1 In Vitro, and the IKKε-mediated Phosporylation
of IRF3 In Situ (i.e., In HEK293T Cells in Culture).

| | | | pIRF3 |
|---|---|---|---|
| Example | IKKε | TBK1 | ELISA |
| Compound | IC50 | IC50 | IC50 |
| No. | (μM) | (μM) | (μM) |
| 2 | 0.0011 | 0.0004 | 0.0719 |
| 3 | 0.0028 | 0.0002 | 0.2800 |
| 59 | 0.0002 | 0.0004 | 0.0162 |
| 80 | 0.0008 | 0.0004 | N/D |
| 93 | 0.0007 | 0.0003 | 0.1390 |
| 176 | 0.0009 | 0.0003 | 0.0125 |
| 190 | 0.0004 | 0.0003 | N/D |
| 264 | 0.0006 | 0.0002 | 0.6538 |
| 381 | 0.0006 | 0.0004 | 0.3490 |
| 392 | 0.0002 | 0.0002 | 0.0122 |
| 439 | 0.0004 | 0.0003 | 0.0080 |
| 467 | 0.0035 | 0.0002 | N/D |
| 490 | 0.0014 | 0.0003 | N/D |
| 499 | 0.0051 | 0.0005 | 0.1417 |
| 500 | 0.0020 | 0.0003 | 0.0918 |
| 501 | 0.0011 | 0.0001 | 0.0259 |
| 534 | 0.0008 | 0.0004 | N/D |
| 536 | 0.0003 | 0.0013 | 0.0619 |
| 543 | 0.0011 | 0.0013 | 0.0503 |
| 561 | 0.0004 | 0.0007 | 0.0560 |
| 565 | 0.0006 | 0.0021 | N/D |
| 585 | 0.0004 | 0.0019 | N/D |
| 588 | 0.0003 | 0.0003 | 0.0310 |
| 590 | 0.0003 | 0.0012 | N/D |
| 594 | 0.0003 | 0.0020 | >5 |
| 596 | 0.0007 | 0.0010 | N/D |
| 601 | 0.0024 | 0.0004 | 0.2760 |
| 611 | 0.0015 | 0.0006 | N/D |
| 613 | 0.0008 | 0.0004 | 0.0199 |
| 616 | 0.0011 | 0.0016 | 0.0985 |
| 619 | 0.0012 | 0.0002 | 0.2872 |
| 620 | 0.0007 | 0.0019 | 0.0503 |
| 625 | 0.0020 | 0.0004 | 0.0169 |
| 627 | 0.0030 | 0.0013 | 1.1030 |
| 631 | 0.0008 | 0.0013 | 0.0319 |
| 632 | 0.0005 | 0.0004 | 0.0228 |
| 646 | 0.0007 | 0.0013 | 0.0309 |
| 648 | 0.0039 | 0.0005 | 0.1786 |
| 657 | 0.0013 | 0.0013 | 0.0976 |

N/D = not determined

### ELISA to Detect Secreted RANTES

Prostate cancer DU145 cells were seeded at 20,000 cells/well in a 96-well tissue culture plate. The following day media was removed and replaced with complete media containing IKKε/TBK1 inhibitor (starting concentration 25 M, 1:3 dilutions, final DMSO 0.05%). Cells were incubated for 20 hours and culture supernatant used to determine secreted RANTES levels using a commercially available ELISA kit (R & D Systems, Minneapolis, Minn.).

An alternative method was also developed to monitor Poly(I:C) (Sigma-Aldrich, St. Louis, Mo.) induced RANTES production in human fibroblast cells, MALME-3 (American Type Tissue Collection, Manassas, Va.). Cells were seeded at 2500 per well in a 96-well plate and the following day media was removed and replaced with complete media containing various concentrations of compound. One hour post-compound addition cells were treated with 100 ug/ml Poly(I:C) and the following day supernatant was collected and analyzed using the human RANTES ELISA kit as described above.

Many compounds according to Formula I were found to inhibit the secretion of RANTES with an IC₅₀ of about 10 nM or less using this assay. For example, Example Compounds 446, 492, and 505 inhibited the secretion of RANTES with an IC₅₀ of less than about 10 nM.

### Inhibition of RANTES and IP-10 Production by Human Fibroblast-Like Synoviocytes from Patients with Rheumatoid Arthritis

### Introduction:

Rheumatoid arthritis (RA) synovial cells have upregulated IKKε, IRF3, RANTES, and IP-10 levels. IKKε knockout mice have moderately reduced arthritis and reduced levels of the above mentioned proteins. Treatment of human fibroblast like synoviocyte (HFLS) cells isolated from RA patients with Poly(I:C) mimics the diseased state of RA cells. If pretreatment of HFLS cells with compounds according to Formula I inhibits production of RANTES and IP-10 chemokines in response to Poly(I:C) stimulation, such compounds have therapeutic potential in treating patients with RA.

### Protocol:

HFLS cells (HFLS-RA) isolated from patients with rheumatoid arthritis were obtained from Cell Applications, Inc. (San Diego, Calif.). Cells were seeded in synoviocyte growth medium (Cell Applications, Inc., San Diego, Calif.) and allowed to grow overnight. The following day, media was replaced and cells were treated with varying concentrations of selected compounds according to Formula I (e.g., Example Compound 5) (0.1% final DMSO concentration). Two hours later, cells were induced with 50 g/mL Poly(I:C) (Sigma-Aldrich, St. Louis, Mo.). Supernatants were collected 20 hours post-induction and used to monitor RANTES and IP-10 levels using DuoSet ELISA kits (Human CXCL10/IP-10 DuoSet & Human CCL5/RANTES DuoSet; R&D Systems, Inc., Minneapolis, Minn.).

### Results:

Pretreatment of HFLS cells with a compound according to Formula I was found to inhibit production of RANTES and IP-10 chemokines from these cells using this assay. Specifically, Compound 5 was found to inhibit production of RANTES and IP-10 with an IC₅₀ of about 60 nM. Using a similar assay Compound 5 was also found to inhibit production of IFN-β with an IC₅₀ of about 40 nM

### Identification of Genes Modulated by IKKε/TBK1 Inhibition in HFLS-RA Cells

### Introduction:

IKKε and TBK1 play important roles in modulating several innate/adaptive immune and interferon-regulated genes in response to bacterial and viral infections. To identify genes that are under the control of IKKε and TBK1 kinase activity HFLS-RA cells (Cell Applications, Inc., San Diego, Calif.) were pretreated with a compound according to Formula I (Example Compound 5) (0.5 uM), and then treated with the TLR3 agonist Poly(I:C). A focused RT-PCR array containing either 84 innate/adaptive immune-regulated or 84 IFNα/β-regulated genes were probed by qRT-PCR using mRNA isolated from the treated cells, as well as from untreated control cells, according to the following protocol.

### Protocol:

HFLS cells isolated from patients with RA were obtained from Cell Applications, Inc. (HFLS-RA, Cell Applications, Inc., San Diego, Calif.). Cells were seeded in synoviocyte growth medium (Cell Applications, Inc., San Diego, Calif.) and allowed to grow overnight. The following day, media was replaced and cells were treated with 500 nM of Example Compound 5 (0.1% final DMSO concentration). Two hours later, cells were induced with 50 g/mL Poly(I:C) (Sigma-Aldrich, St. Louis, Mo.). Cells were harvested 5 hours later and total RNA was isolated and processed using the RNeasy Mini Kit, QIAshredder and RNase-Free DNase Set (all from Qiagen, Inc., Valencia, Calif.). RNA was quantitated using Quant-iT™ RiboGreen® RNA Assay Kit (Invitrogen, Inc., Carlsbad, Calif.). First strand cDNA was synthesized using RT² First Strand Kit (SABiosciences, Frederick, Md.). Real time PCR-based gene expression analysis was performed on the Human Innate & Adaptive Immune Responses (SABiosciences, Frederick, Md.) and the Human Interferon α/β Response Arrays (SABiosciences, Frederick, Md.) using the 7300 Real-Time PCR System (Applied Biosytems, Foster City, Calif.). To confirm gene modulation, TaqMan Gene Expression Assay probes CASP-1, IFN-β, IRF1, TLR3, MYD88, and GAPDH were purchased from Applied Biosystems, Inc. (Foster City, Calif.) and run on the ABI-7300 Real-Time PCR System (Applied Biosystems, Inc., Foster City, Calif.).

### Conclusion:

The induction of genes normally induced by Poly(I:C) treatment was potently inhibited by pre-treatment with Compound 5. Such inhibition of proinflammatory cytokine and chemokine production suggests that the compounds according to Formula I may used to treat, or lessen the symptoms of rheumatoid arthritis.

### Cell Growth Inhibition Assays

DU4475, COLO205, and OPM2 cells were plated in 96-well plates at 5000 cells/well. The following day test compounds (compounds according to Formula I) were added, maintaining the final DMSO solvent concentration at 0.4%. After the desired incubation time (3-5 days), cell number was assayed using the CellTiter-Glo luminescent cell viability assay (Promega, Inc., Madison, Wis.). Viability was expressed as percent DMSO control after background subtraction.

Using the assays described above Example Compound numbers 127, 316 and 339 were found to inhibit the growth of DU4475 cells with an IC₅₀ of about 10 nM or less.

### Glucose Uptake Assay Using Differentiated 3T3-L1 Adipocytes

Studies have demonstrated that IKKε knockout mice exhibit reduced weight gain and less complications associated with diabetes compared to wild type mice under high-fat diet conditions (Chiang et al.; The protein kinase IKKε regulates energy balance in obese mice; Cell, 138:961-975, 2009). To determine if IKK/TBK1 inhibitors prevent fatty acid induced insulin resistance in 3T3-L1 adipocytes, insulin-stimulated glucose uptake in the presence of compounds according to Formula I was monitored.

Murine 3T3-L1 cells were differentiated to adipocytes in 96-well plates by incubating for 2 days in adipogenic cocktail (10 ug/ml insulin, 115 ug/ml isobutylmethylxanthine, 1 uM dexamethasone) followed by incubation in insulin-supplemented medium for 2 days and complete media for an additional 5-10 days. Adipocytes were treated with BSA-complexed palmitic acid and a compound according to Formula I for 48 hours. Following free fatty acid treatment, adipoctyes were insulin-deprived in serum-free media for 2 hours. Subsequently, the media was replaced with KRH buffer containing a compound according to Formula I and 300 nM insulin for 15-20 minutes. [¹⁴C]-labeled 2-deoxyglucose was then added for 15 minutes. Cells were thoroughly washed with ice-cold PBS, and intracellular [¹⁴C]-2-deoxyglucose was measured in cell lysates by scintillation.

In this cell culture model of obesity-induced insulin resistance, Compound 5 was found to reverse the inhibitory effects of free fatty acid on insulin-stimulated glucose uptake. These results suggest that compounds according to Formula I have the potential to alleviate obesity-mediated insulin resistance.

### Evaluation of Example Compound 5 in a Collagen-Induced Arthritis Model in Mice

### Protocol

Male DBA/1 mice were injected with 150 μL of 2 mg/kg bovine type II collagen in Freund's complete adjuvant on days 0 and 21. On days 18 through 34, 100 mg/kg or 150 mg/kg Example Compound 5 was administered orally each day. Also on days 18 through 34, all mouse paws were given a clinical score on a scale of 0-5, based upon the severity of erythema and swelling. Body weights were measured every other day beginning on day 18. Mice were euthanized on day 34, livers were weighed and paws frozen in preparation for subsequent histopathology evaluation.

### Results

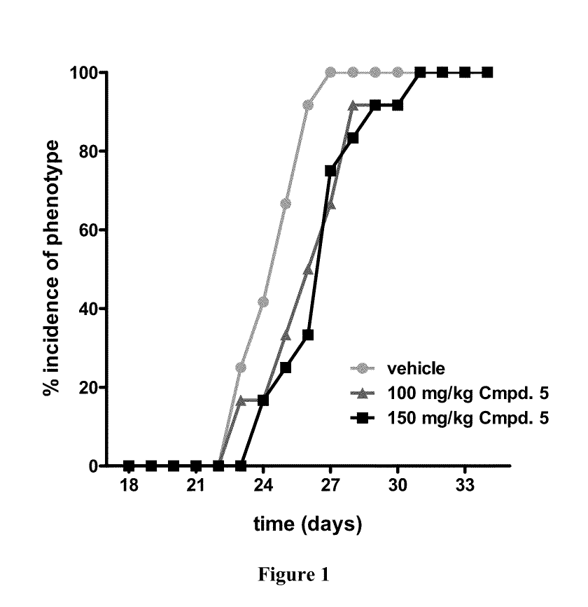

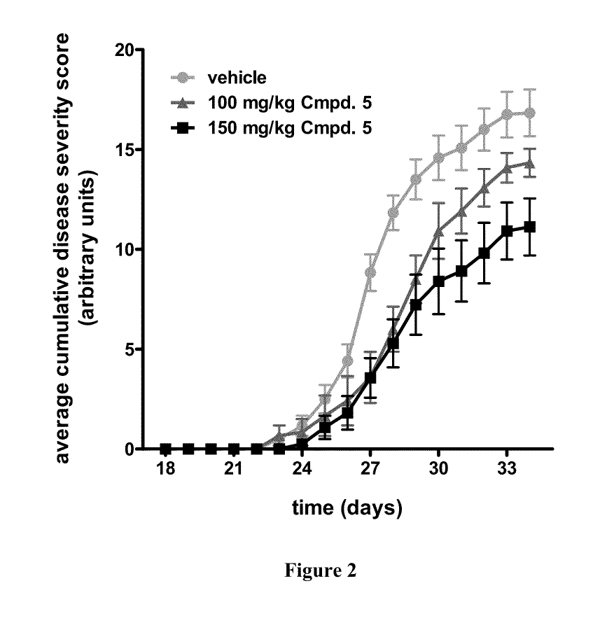

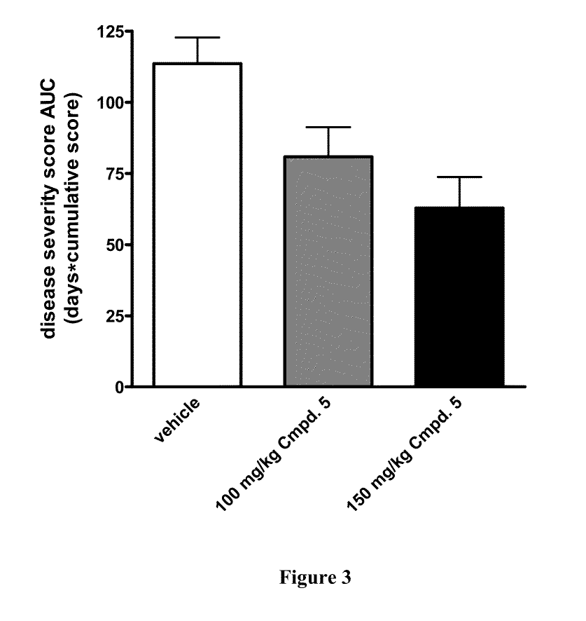

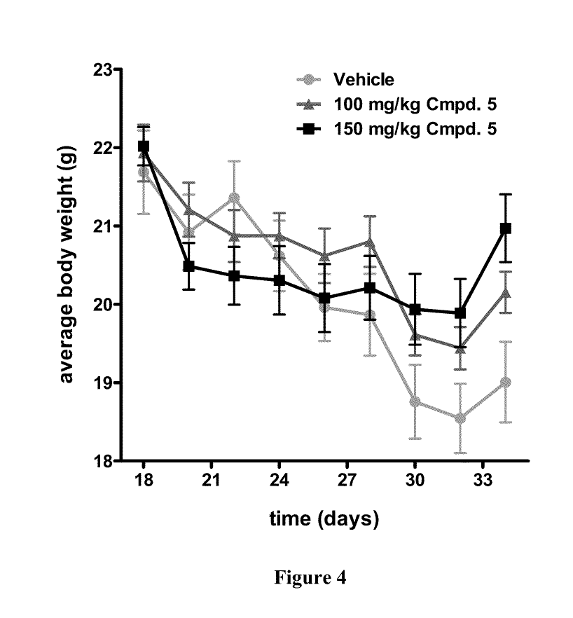

In vehicle-treated, immunized mice, symptoms of arthritis first appeared on day 23 and were present in all mice by day 27. In mice treated with Compound 5, symptoms appeared on day 23 and 24 for 100 mg/kg and 150 mg/kg respectively, and were present in all mice by day 30 for both doses (FIG. 1). This drug-related delay was also evident in the rate of increase in clinical score. Expressed as the cumulative clinical score for the all paws of each mouse, increases in erythema and swelling were significantly slower with both doses of Compound 5. Furthermore, the magnitude of clinical score on day 34 was reduced 20% (p<0.03) and 38% (p<0.006) for 100 and 150 mg/kg, respectively (FIG. 2). The AUC values for clinical score as a function of time showed even greater drug effects overall, with 29% (p=0.01) and 45% (p<0.002) inhibition by 100 mg/kg and 150 mg/kg Compound 5, respectively (FIG. 3). Vehicle-treated, immunized mice lost an average of 2.7 g or 12% of their body weight from day 18-34. With 100 mg/kg and 150 mg/kg Compound 5, body weight loss was inhibited 23% (p=0.04) and 42% (p<0.001), respectively (FIG. 4). No differences in liver weights were observed for any treatment (data not shown). Histopathological analysis of joints remains to be completed.

### Conclusions

Example Compound 5 showed significant, dose-dependent effects in reducing the collagen-induced arthritis in this mouse model. Both the rate of disease progression and magnitude of disease severity were inhibited. Mice administered Compound 5 lost less weight, consistent with decreased severity of disease. Anti-type II collagen antibody titers were not determined; therefore, the extent to which the activity of Compound 5 was due to effects on inflamed joint tissues directly, or through possible reduction in antibody titer, remains to be determined. Based upon suppression of cytokine and chemokine production observed with in human RA synoviocytes and other immune cell types treated with Compound 5 in culture, it is likely that direct effects on joint tissues is at least partially responsible for the suppression of the arthritic phenotype by Compound 5 in mice.

### IKKε/TBK1 Inhibition in RAW264.7 Mouse Cells Prevents Induction of RANTES and IFN-β after Treatment with Nucleic Acid Agonists

### Introduction:

Mouse RAW264.7 macrophage-like cells provide a model for macrophage function in tissue culture. To investigate the efficacy of compounds according to Formula I in inhibiting nucleic acid cytosolic receptor pathways RAW264.7 cells were pretreated with a compound according to Formula I (Example Compound 471) and then exposed to various single stranded and double stranded RNA and DNA agonists introduced into the cell. To track IKKε/TBK1 signaling pathway activation, RANTES or IFN-β protein secretion was monitored by ELISA-based assays (R & D systems), such as those described above.

### Protocol:

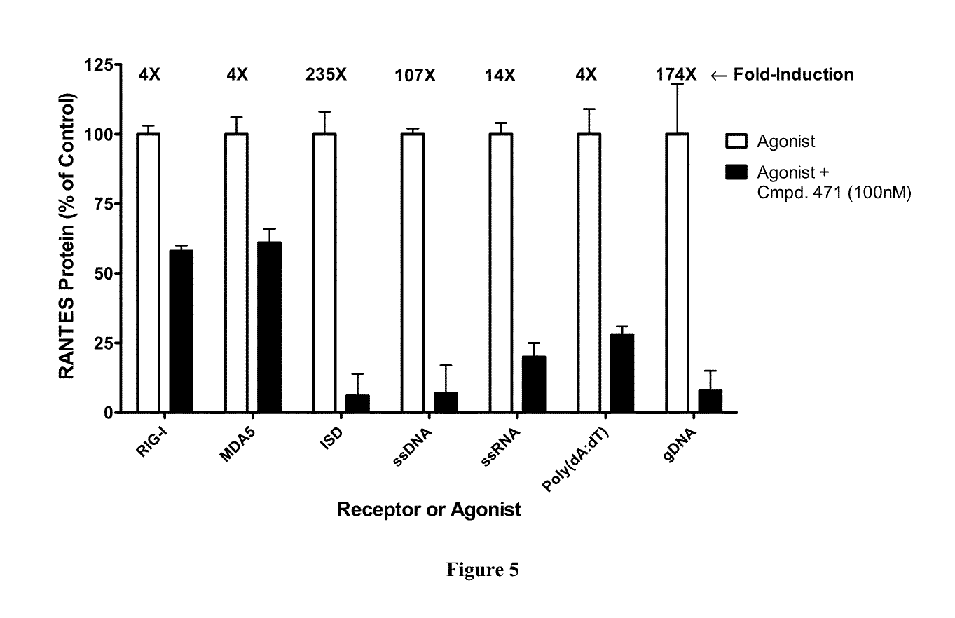

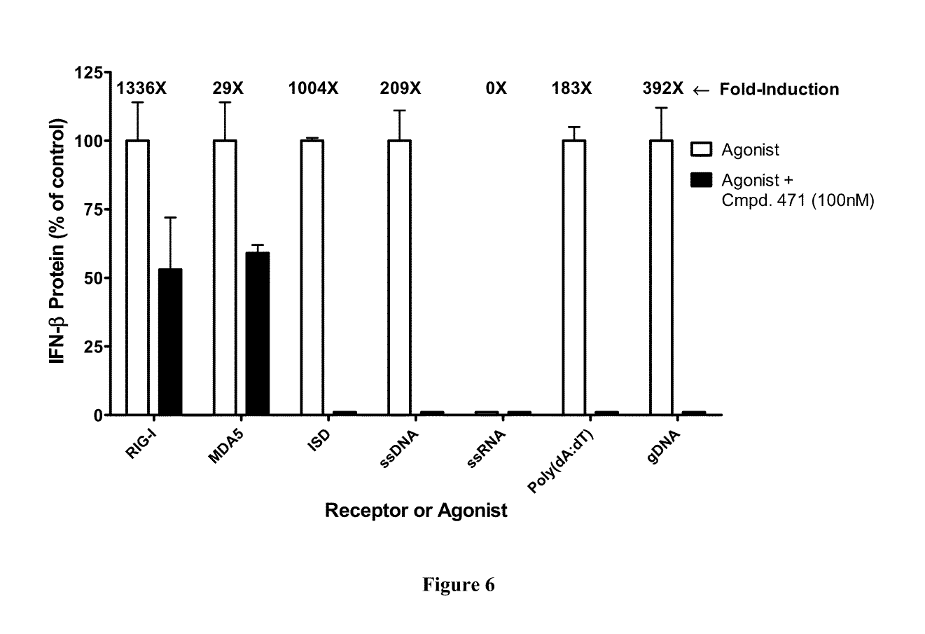

RAW264.7 cells were seeded in 96-well culture plates and allowed to grow overnight. The following day, media was replaced and cells were pretreated with 100 nM Example Compound 471 (0.1% final DMSO concentration). After one hour cells were transfected with Lipofectime LTX reagent (Invitrogen, Carlsbad, Calif.) and one of the following agonists: low molecular weight Poly(I:C) (InvivoGen, San Diego, Calif.) at 10 μg/ml to activate RIG-I; high molecular weight Poly(I:C) (InvivoGen, San Diego, Calif.) at 10 μg/ml to activate MDA5; Poly(dA:dT) (InvivoGen, San Diego, Calif.) at 1 ug/ml; 45-basepair double stranded interferon stimulatory DNA oligo (ISD) at 10 μg/ml (Stetson and Medzhitov; Recognition of cytosolic DNA activates an IRF3-dependent innate immune response; Immunity, 24:93-103, 2006); ssDNA at 10 μg/ml (InvivoGen, San Diego, Calif.), ssRNA at 0.5 μg/ml (InvivoGen, San Diego, Calif.), or salmon sperm genomic DNA (gDNA) (InvivoGen, San Diego, Calif.) at 10 ug/ml to activate DAI, IFI16, and other cytosolic nucleic acid receptors. RANTES (FIG. 5) and IFN-β (FIG. 6) secretion were quantified using ELISA kits (Mouse CCL5/RANTES, R&D Systems, Inc., Minneapolis, Minn. and Mouse IFN-β, Thermo Fisher Scientific, Rockford, Ill.).

### Results:

The low molecular weight and high molecular weight poly(I:C) induced both RANTES (FIG. 5) and IFN-β (FIG. 6) protein secretion and that induction of secretion was modestly inhibited with compound 471 at 100 nM. The double and single stranded DNA agonists; ISD, ssDNA, poly(dA:dT), and gDNA, all potently induced RANTES (FIG. 5) and IFN-β (FIG. 6) secretion, and that induction of secretion was potently inhibited by treatment with compound 471 at 100 nM. The ssRNA agonist also induced RANTES secretion, and that induction of secretion was potently inhibited by compound 471 at 100 nM (FIG. 5), but the ssRNA agonist did not induce IFN-β secretion in RAW264.7 cells (FIG. 6).

### Conclusion:

The inhibition of IKKε and/or TBK1 with small molecule inhibitors potently reduces secreted levels of IFN-β and RANTES after transfection of single or double stranded RNA and DNA molecules. Inhibition of secretion of key proinflammatory cytokines, such as IFN-β and RANTES may be useful for the treatment of various autoimmune diseases as described above.

### Modulation of Agonist Induced Genes in Normal and SLE PBMCs

To determine if inhibition of IKKε and/or TBK1 modulates nucleic acid agonist induced gene expression, high molecular weight poly(I:C) (MDA5 agonist) and low weight poly(I:C) (RIG-I agonist) were electroporated into human peripheral blood mononuclear cells (PBMCs) obtained from normal donors, or low molecular weight Poly(I:C) was electroporated into PBMCs from donors that have Systemic Lupus Erythematosus (SLE). Induction of IFN-α2, IFN-β, and BLyS mRNA production was monitored by qRT-PCR.

### Protocol

Human PBMCs were collected from healthy donors using routine laboratory procedures. PBMCs from SLE patients were purchased from Astarte Biologics (Redmond, Wash.). The PBMCs were electroporated using Nucleofector® Kit V (Lonza, Walkersville, Md.) with 0.4 ug/mL of high molecular weight poly (I:C) (InvivoGen, San Diego, Calif.) or 0.4 ug/mL low molecular weight poly (I:C) (InvivoGen, San Diego, Calif.) and seeded into wells containing serial dilutions of Example Compound 5 (0.1% final DMSO concentration). Cells were harvested 4 hours post-electroporation and total RNA was isolated and processed using RNeasy Mini Kit, QIAshredder, and RNase-Free DNase Set (all from Qiagen, Germantown, Md.). RNA was quantitated using Quant-iT™ RiboGreen® RNA Assay Kit (Invitrogen, Carlsbad, Calif.). Reverse transcription and real-time PCR were performed using the QuantiTect Probe RT-PCR Kit (Qiagen, Germantown, Md.) and the 7300 Real-Time PCR System (Applied Biosytems, Foster City, Calif.). Probe sets, IFN-α2, IFN-β1, BLyS, and GAPDH used for normalization, were all purchased from Applied Biosystems, Inc (Carlsbad, Calif.).

### Conclusion

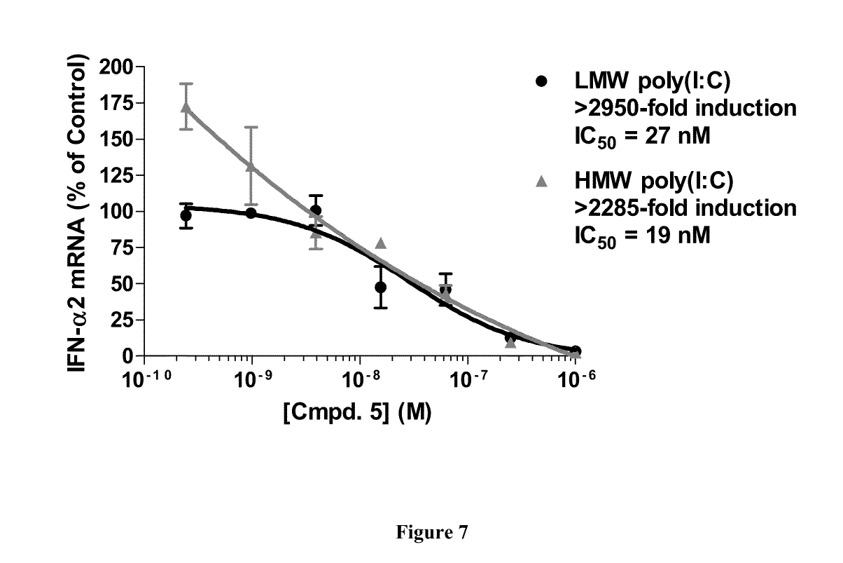

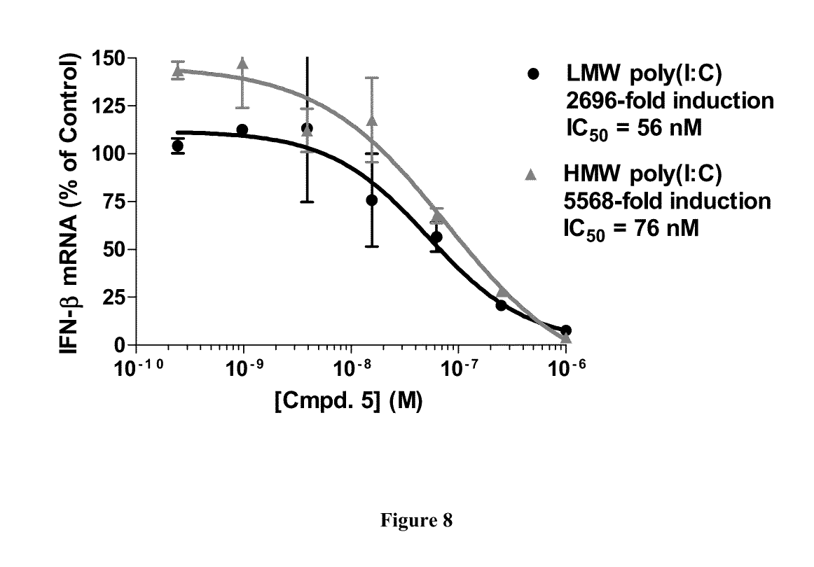

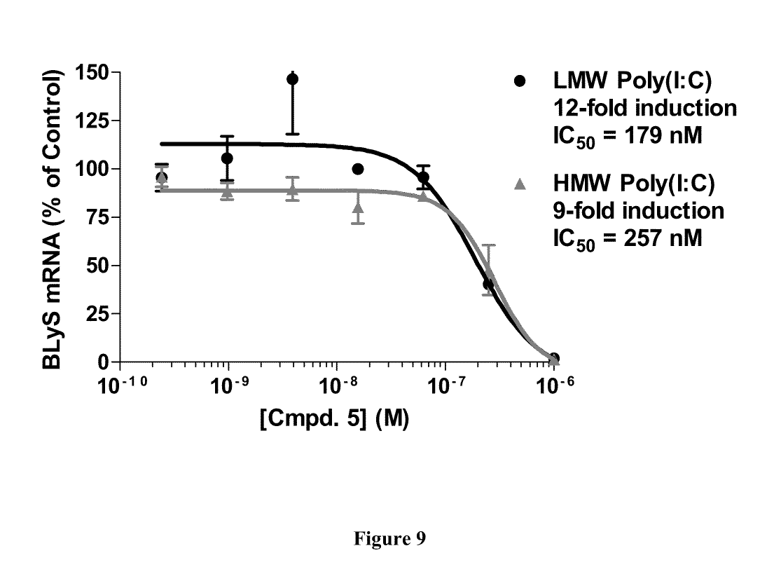

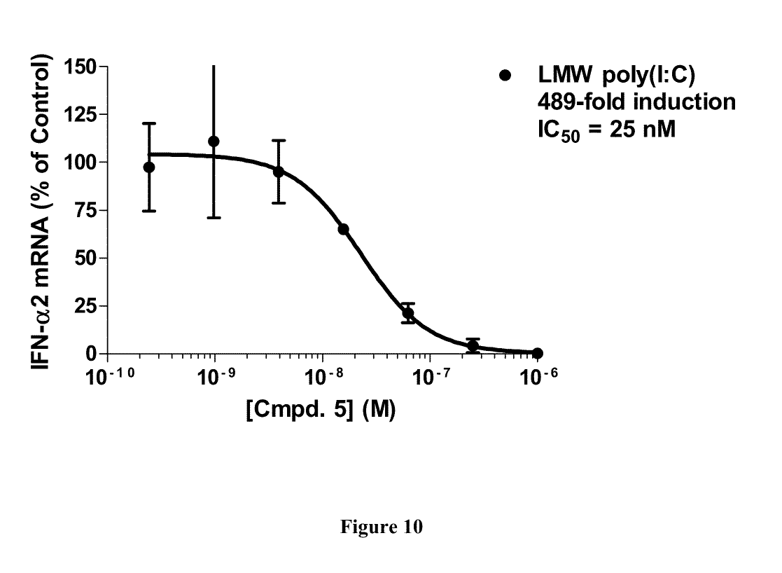

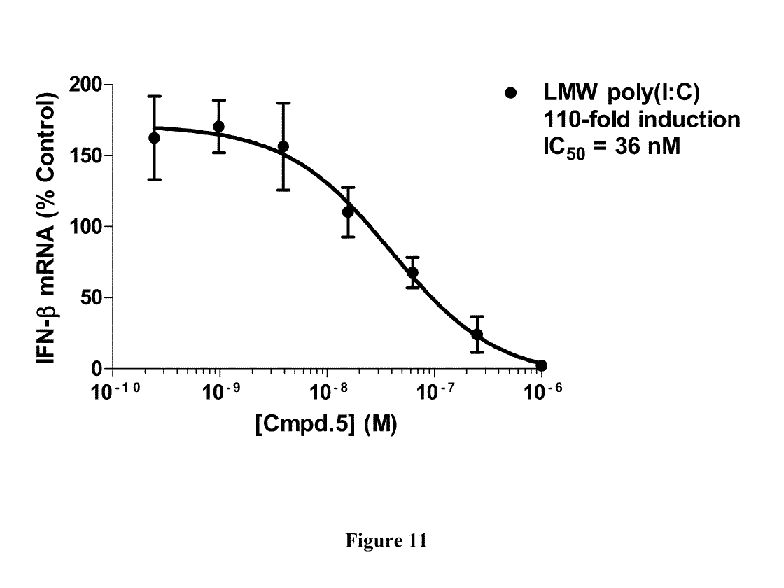

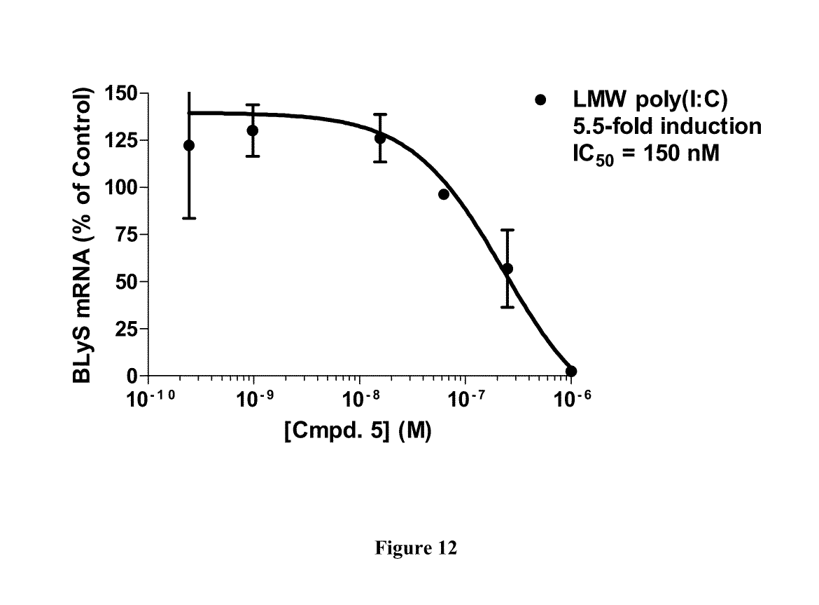

PBMC samples from both normal (FIGS. 7, 8 and 9) and SLE patients (FIGS. 10, 11 and 12) showed robust induction of IFN-α2 (FIGS. 7 and 10), IFN-β1 (FIGS. 8 and 11), and BLyS (FIGS. 9 and 12) mRNAs after LMW poly(I:C) agonist treatment. The induction of IFN-α2 (FIGS. 7 and 10), IFN-β1 (FIGS. 8 and 11), and BLyS (FIGS. 9 and 12) mRNAs was potently inhibited by Compound 5 in a dose-dependent manner. Treatment of normal PBMCs with HMW poly(I:C) showed a similar response to the LMW studies. These results suggest that activation of RIG-I and MDA5 receptors and IKKε/TBK1 pathway dependent induction of type I interferons (IFN-α2 and IFN-β1), as well as downstream interferon-signature genes (e.g. BLyS), are dramatically reduced by treatment with Compound 5. These results further suggest that compounds according to Formula I can be used to limit flare ups and other complications in SLE patients arising from elevations in nucleic acid agonists.

All publications and patent applications mentioned in the specification are indicative of the level of those skilled in the art to which the present invention pertains. The mere mentioning of the publications and patent applications does not necessarily constitute an admission that they are prior art to the instant application.

Although the foregoing invention has been described in some detail by way of illustration and example for purposes of clarity of understanding, it will be clear to the skilled artisan that certain changes and modifications may be practiced within the scope of the appended claims.

What is claimed is:

1. A method of inhibiting TANK Binding Kinase 1 (TBK1) comprising administering a compound having a structure according to the following: 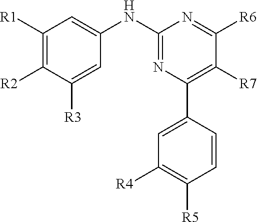
and pharmaceutically acceptable salts thereof, wherein:
R1, R2, R3, and R5 are independently chosen from the following groups: alkyl, alkylene, alkenyl, alkenylene, alkynyl, carbocycle, cycloalkyl, cycloalkenyl, heterocycle, aryl, heteroaryl, halo, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, cycloalkylthio, arylalkyl, heteroarylalkyl, heteroarylalkenyl, arylalkynyl, haloalkyl, aldehyde, thiocarbonyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, aminothiocarbonyl, hydroxyaminocarbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide,
wherein any of the foregoing groups are optionally substituted at least once with alkyl, alkylene, alkenyl, alkenylene, alkynyl, carbocycle, cycloalkyl, cycloalkenyl, heterocycle, aryl, heteroaryl, halo, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, cycloalkylthio, arylalkyl, heteroarylalkyl, heteroarylalkenyl, arylalkynyl, haloalkyl, aldehyde, thiocarbonyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, aminothiocarbonyl, hydroxyaminocarbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide,
R4 is independently chosen from hydro, halo, and an optionally-substituted group chosen from lower alkyl, haloalkyl, alkoxy, arylalkoxy, heteroarylalkoxy, and heterocycloalkoxy;
R6 and R7 are independently chosen from hydro, halo, and lower alkyl; or
R6, taken together with R7, form an aryl or heteroaryl ring.

2. A method of treating a diseases associated with TANK Binding Kinase 1 (TBK1), the method comprising administering a compound having a structure according to the following: 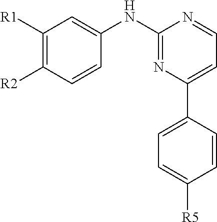
and pharmaceutically acceptable salts thereof, wherein:
R1 is chosen from hydro or methoxy;
R2 is chosen from the following groups: heterocycle, alkoxy, heterocycloxy, amino, C-amido, N-amido, and sulfonamide, wherein any of the foregoing groups are optionally substituted at least once with alkyl, alkylene, alkenyl, alkenylene, alkynyl, carbocycle, cycloalkyl, cycloalkenyl, heterocycle, aryl, heteroaryl, halo, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, cycloalkylthio, arylalkyl, heteroarylalkyl, heteroarylalkenyl, arylalkynyl, haloalkyl, aldehyde, thiocarbonyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, aminothiocarbonyl, hydroxyaminocarbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide; and
R5 is chosen from the following groups: alkyl, halo, hydroxyl, alkoxy, aryloxy, arylalkoxy, amino, C-amido, and N-amido, wherein any of the foregoing groups are optionally substituted at least once with alkyl, alkylene, alkenyl, alkenylene, alkynyl, carbocycle, cycloalkyl, cycloalkenyl, heterocycle, aryl, heteroaryl, halo, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, cycloalkylthio, arylalkyl, heteroarylalkyl, heteroarylalkenyl, arylalkynyl, haloalkyl, aldehyde, thiocarbonyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, aminothiocarbonyl, hydroxyaminocarbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide.

3. The method of claim 2, wherein the disease is selected from the group of: an autoimmune disease, rheumatoid arthritis, systemic lupus erythematosus, diseases associated with aberrant accumulation of cytosolic nucleic acids, Sjögrens syndrome, Aicardi-Goutières syndrome, chilblain lupus, retinal vasculopathy and cerebral leukodystrophy, systemic sclerosis, myositis, dermatomyositis, polymyositis, psoriasis, chronic obstructive pulmonary disease, inflammatory bowel disease, obesity, insulin resistance, non-insulin-dependent diabetes mellitus, metabolic syndrome, and a cancer.

4. The method of claim 2, wherein the disease is a cancer.

5. The method of claim 2, wherein the disease is an autoimmune disease.

6. The method of claim 2, wherein the disease is rheumatoid arthritis.

7. The method of claim 2, wherein the disease is lupus.

8. A method of inhibiting TANK Binding Kinase 1 (TBK1) in a cell, the method comprising contacting the cell with a compound having a structure according to the following: 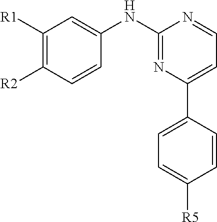
and pharmaceutically acceptable salts thereof, wherein:
R1 is chosen from hydro or methoxy;
R2 is chosen from heterocyclyl, morpholinyl, methoxy, or amino; and
R5 is chosen from the following groups: alkyl, halo, alkoxy, alkanoyl, benzyloxy, carbonyl, C-carboxy or C-amido.

9. The method of claim 8, wherein R5 is C-carboxy.

10. The method of claim 9, wherein R2 is morpholinyl.

11. The method of claim 9, wherein R2 is amino.

12. The method of claim 8, wherein R5 is C-amido.

13. The method of claim 12, wherein R2 is morpholinyl.

14. The method of claim 12, wherein R2 is amino.

15. The method of claim 8, wherein R2 is morpholinyl.

16. The method of claim 8, wherein R2 is amino.
